(19) United States
(12) Patent Application Publication
(10) Pub. No.: US 2026/0217826 A1
(43) Pub. Date: Jul. 30, 2026

(54) TCR-VBETA SPECIFIC THERAPEUTIC MOLECULES AND USES THEREOF

(71) Applicant: YALE UNIVERSITY, New Haven, CT (US)

(72) Inventors: Michael GIRARDI, Madison, CT (US); Jingjing REN, Branford, CT (US); Xiaofeng LIAO, Branford, CT (US); Julia Mae LEWIS, Clinton, CT (US)

(21) Appl. No.: 19/143,580

(22) PCT Filed: Dec. 27, 2023
(86) PCT No.: PCT/US2023/086113

§ 371 Date: Jun. 26, 2025

Related U.S. Application Data
(60) Provisional application No. 63/477,365, filed on Dec. 27, 2022.
(60) Provisional application No. 63/484,916, filed on Feb. 14, 2023.

(51) Int. Cl. C07K 16/28; A61K 40/11; A61K 40/31; A61K 40/42; A61P 35/00; C12N 15/86

(52) U.S. Cl. CPC C07K 16/2809; A61K 40/11; A61K 40/31; A61K 40/421; A61P 35/00; C12N 15/86; C07K 2317/24; C07K 2317/56; C07K 2317/565; C07K 2317/622; C07K 2317/732; C12N 2750/14143

(57) ABSTRACT

Provided are compositions and uses for binding a chimeric antigen receptor (CAR) or an antibody or antigen binding fragment to a Vβ region of a T cell receptor. Also provided are methods for treating a disease such as cancer or an autoimmune disease using Vβ region targeting molecules.

### CROSS REFERENCE TO RELATED APPLICATIONS

The present application claims the priority benefit of U.S. Provisional Application No. 63/477,365, filed Dec. 27, 2022, and U.S. Provisional Application No. 63/484,916, filed Feb. 14, 2023, which are hereby incorporated by reference in their entirety.

### REFERENCE TO SEQUENCE LISTING SUBMITTED ELECTRONICALLY VIA EFS-WEB

The content of the electronically submitted sequence listing (Name: 2681_136PC02_Seqlisting_ST26.xml; Size: 858,459 bytes; and Date of Creation: Dec. 26, 2023), filed with the application, is incorporated herein by reference in its entirety.

### FIELD

According to various aspects, the present disclosure relates to a chimeric antigen receptor (CAR) or antibody or antigen-binding fragment comprising a domain that binds to a Vβ region of a T-cell receptor. The disclosure also relates to methods of treating a disease in a subject using such binding molecules.

### BACKGROUND

Autoimmune diseases are caused by dysfunctional T cells (Haroon N et al., Arthritis Rheum. 2013 October; 65(10):2645-54, Duarte J. et al., PloS One 2010 May 10; 5(5):e10558; Konig M. et al., Front Immunol 2016 Jan. 25; 7:11). Dysfunctional T cells afflict a larger population than those solely affected by autoimmune diseases and encompass individuals having cancer and related T cell malignancies. Lymphocyte malignancies, including lymphocytic leukemias and lymphomas, can largely be divided into those which are derived from either T cells or B cells. T cell malignancies are a clinically and biologically heterogeneous group of disorders, together comprising 10-20% of non-Hodgkin's lymphomas and 20% of acute leukemia (see, e.g., https://www.cancer.org/cancer/non-hodgkin-lymphoma/about/t-cell-lymphoma.html).

A difficulty in the development of a treatment for T cell disorders and T cell malignancies is the considerable overlap in marker expression of clonal and normal T-cells, with no single antigen clearly able to identify clonal (malignant) cells. Chimeric antigen receptor (CAR) T-cell therapy has shown efficacy in B cell leukemias and lymphomas. However, targeting T cell lymphoproliferative disorders has been challenging due to the difficulty in identifying appropriate antigens for CAR cell therapy. Further, there is no satisfactory way to mitigate loss of T-cells without depleting the T cell count, which leads to undesirable toxicity and severe immunosuppression.

A need exists for methods of treating T cell mediated diseases and T cell malignancies, which exhibit high specificity and efficacy in the elimination of abnormal T cells.

### BRIEF SUMMARY

The present disclosure provides a chimeric antigen receptor (CAR) comprising (i) an antigen binding molecule that specifically binds to a TCR Vβ region, (ii) an extracellular domain, (iii) a transmembrane domain, (iv) a costimulatory domain and (v) an activating domain, wherein the antigen binding molecule is a single chain variable fragment (scFv) comprising: a variable heavy chain (VH) according to any one of SEQ ID NOs: 45-131 and 534-544, and/or a variable light chain (VL) according to any one of SEQ ID NOs: 132-227 and 545-558.

In some aspects, (i) the variable heavy chain (VH) comprises CDR1, CDR2 and CDR3 according to SEQ ID NO:10, SEQ ID NO:11, and SEQ ID NO:12, respectively, and/or (ii) the variable light chain (VL) comprises CDR1, CDR2 and CDR3 according to SEQ ID NO:13, SEQ ID NO:14, SEQ ID NO:15, respectively; or (i) the variable heavy chain (VH) comprises CDR1, CDR2 and CDR3 according to SEQ ID NO:370, SEQ ID NO:371, and SEQ ID NO:372, respectively, and/or (ii) the variable light chain (VL) comprises CDR1, CDR2 and CDR3 according to SEQ ID NO:418, SEQ ID NO:419, SEQ ID NO:420, respectively; or (i) the variable heavy chain (VH) comprises CDR1, CDR2 and CDR3 according to SEQ ID NO:373, SEQ ID NO:374, and SEQ ID NO:375 respectively, and/or (ii) the variable light chain (VL) comprises CDR1, CDR2 and CDR3 according to SEQ ID NO:421, SEQ ID NO:422, SEQ ID NO:423, respectively; or (i) the variable heavy chain (VH) comprises CDR1, CDR2 and CDR3 according to SEQ ID NO:376, SEQ ID NO:377, and SEQ ID NO:378, respectively, and/or (ii) the variable light chain (VL) comprises CDR1, CDR2 and CDR3 according to SEQ ID NO:424, SEQ ID NO:425, SEQ ID NO:426, respectively; or the variable heavy chain (VH) comprises CDR1, CDR2 and CDR3 according to SEQ ID NO:379, SEQ ID NO:380, and SEQ ID NO:381 respectively, and/or (ii) the variable light chain (VL) comprises CDR1, CDR2 and CDR3 according to SEQ ID NO:427, SEQ ID NO:428, SEQ ID NO:429, respectively; or (i) the variable heavy chain (VH) comprises CDR1, CDR2 and CDR3 according to SEQ ID NO:382, SEQ ID NO:383, and SEQ ID NO:384, respectively, and/or (ii) the variable light chain (VL) comprises CDR1, CDR2 and CDR3 according to SEQ ID NO:430, SEQ ID NO:431, SEQ ID NO:432, respectively; or (i) the variable heavy chain (VH) comprises CDR1, CDR2 and CDR3 according to SEQ ID NO:385, SEQ ID NO:386, and SEQ ID NO:387, respectively, and/or (ii) the variable light chain (VL) comprises CDR1, CDR2 and CDR3 according to SEQ ID NO:433, SEQ ID NO:434, SEQ ID NO:435, respectively; or (i) the variable heavy chain (VH) comprises CDR1, CDR2 and CDR3 according to SEQ ID NO:388, SEQ ID NO:389, and SEQ ID NO:390, respectively, and/or (ii) the variable light chain (VL) comprises CDR1, CDR2 and CDR3 according to SEQ ID NO:436, SEQ ID NO:437, SEQ ID NO:438, respectively; or (i) the variable heavy chain (VH) comprises CDR1, CDR2 and CDR3 according to SEQ ID NO:391, SEQ ID NO:392, and SEQ ID NO:393 respectively, and/or (ii) the variable light chain (VL) comprises CDR1, CDR2 and CDR3 according to SEQ ID NO:439, SEQ ID NO:440, SEQ ID NO:441, respectively; or (i) the variable heavy chain (VH) comprises CDR1, CDR2 and CDR3 according to SEQ ID NO:394, SEQ ID NO:395, and SEQ ID NO:396, respectively, and/or (ii) the variable light chain (VL) comprises CDR1, CDR2 and CDR3 according to SEQ ID NO:442, SEQ ID NO:443, SEQ ID NO:444, respectively; or (i) the variable heavy chain (VH) comprises CDR1, CDR2 and CDR3 according to SEQ ID NO:397, SEQ ID NO:398, and SEQ ID NO:399, respectively, and/or (ii) the variable light chain (VL) comprises CDR1, CDR2 and CDR3 according to SEQ ID NO:445, SEQ ID NO:446, SEQ ID NO:447, respectively; or (i) the variable heavy chain (VH) comprises CDR1, CDR2 and CDR3 according to SEQ ID NO:400, SEQ ID NO:401, and SEQ ID NO:402, respectively, and/or (ii) the variable light chain (VL) comprises CDR1, CDR2 and CDR3 according to SEQ ID NO:448, SEQ ID NO:449, SEQ ID NO:450, respectively; or (i) the variable heavy chain (VH) comprises CDR1, CDR2 and CDR3 according to SEQ ID NO:403, SEQ ID NO:404, and SEQ ID NO:405 respectively, and/or (ii) the variable light chain (VL) comprises CDR1, CDR2 and CDR3 according to SEQ ID NO:451, SEQ ID NO:452, SEQ ID NO:453, respectively; or (i) the variable heavy chain (VH) comprises CDR1, CDR2 and CDR3 according to SEQ ID NO:406, SEQ ID NO:407, and SEQ ID NO:408, respectively, and/or (ii) the variable light chain (VL) comprises CDR1, CDR2 and CDR3 according to SEQ ID NO:454, SEQ ID NO:455, SEQ ID NO:456, respectively; or (i) the variable heavy chain (VH) comprises CDR1, CDR2 and CDR3 according to SEQ ID NO:409, SEQ ID NO:410, and SEQ ID NO:411, respectively, and/or (ii) the variable light chain (VL) comprises CDR1, CDR2 and CDR3 according to SEQ ID NO:457, SEQ ID NO:458, SEQ ID NO:459, respectively; or (i) the variable heavy chain (VH) comprises CDR1, CDR2 and CDR3 according to SEQ ID NO:412, SEQ ID NO:413, and SEQ ID NO:414, respectively, and/or (ii) the variable light chain (VL) comprises CDR1, CDR2 and CDR3 according to SEQ ID NO:460, SEQ ID NO:461, SEQ ID NO:462, respectively; or (i) the variable heavy chain (VH) comprises CDR1, CDR2 and CDR3 according to SEQ ID NO:415, SEQ ID NO:416, and SEQ ID NO:417, respectively, and/or (ii) the variable light chain (VL) comprises CDR1, CDR2 and CDR3 according to SEQ ID NO:463, SEQ ID NO:464, SEQ ID NO:465, respectively; or (i) the variable heavy chain (VH) comprises CDR1, CDR2 and CDR3 according to SEQ ID NO:584, SEQ ID NO:585, and SEQ ID NO:586, respectively, and/or (ii) the variable light chain (VL) comprises CDR1, CDR2 and CDR3 according to SEQ ID NO:596, SEQ ID NO:597, SEQ ID NO:598, respectively; or (i) the variable heavy chain (VH) comprises CDR1, CDR2 and CDR3 according to SEQ ID NO:587, SEQ ID NO:588, and SEQ ID NO:589, respectively, and/or (ii) the variable light chain (VL) comprises CDR1, CDR2 and CDR3 according to SEQ ID NO:599, SEQ ID NO:601, SEQ ID NO:601, respectively; or (i) the variable heavy chain (VH) comprises CDR1, CDR2 and CDR3 according to SEQ ID NO:590, SEQ ID NO:591, and SEQ ID NO:592, respectively, and/or (ii) the variable light chain (VL) comprises CDR1, CDR2 and CDR3 according to SEQ ID NO:602, SEQ ID NO:603, SEQ ID NO:604, respectively; or (i) the variable heavy chain (VH) comprises CDR1, CDR2 and CDR3 according to SEQ ID NO:593, SEQ ID NO:594, and SEQ ID NO:595, respectively, and/or (ii) the variable light chain (VL) comprises CDR1, CDR2 and CDR3 according to SEQ ID NO:605, SEQ ID NO:606, SEQ ID NO:607, respectively, or SEQ ID NO: 608, SEQ ID NO: 609, SEQ ID NO: 610, respectively.

In some aspects, the variable heavy chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NOs: 45-131 and 534-544.

In some aspects, the variable light chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NO: 132-227 and 545-558.

In some aspects, the TCR Vβ region is selected from the group consisting of Vβ1, Vβ2, Vβ3, Vβ4, Vβ5.1, Vβ5.3, Vβ7.1, Vβ7.2, Vβ8, Vβ9, Vβ11, Vβ12, Vβ13.1, Vβ13.2, Vβ13.6, Vβ14, Vβ16, Vβ17, Vβ18, Vβ20, and Vβ22. In some aspects, the TCR Vβ region is Vβ2.

In some aspects, wherein the antigen-binding domain is a scFv. In some aspects, the scFv comprises an amino acid sequence according to any of SEQ ID NOs: 14-44.

In some aspects, the costimulatory domain comprises a 4-1BB costimulatory domain. In some aspects, the 4-1BB costimulatory domain comprises an amino acid sequence according to SEQ ID NO: 8.

In some aspects, the transmembrane domain comprises an hCD28 transmembrane. In some aspects, the transmembrane domain comprises a hCD28 amino acid sequence according to SEQ ID NO: 7.

In some aspects, the signaling domain comprises a CD247 signaling domain. In some aspects, the signaling domain comprises a CD247 signaling domain amino acid sequence according to SEQ ID NO: 9.

In some aspects, the scFv comprises a CD28 signal peptide. In some aspects, scFv comprises a CD28 signal peptide an amino acid sequence according to SEQ ID NO:3.

In some aspects, the scFv comprises a linker peptide. In some aspects, the scFv comprises a linker peptide according to SEQ ID NO: 4.

The present disclosure also provides an isolated polynucleotide comprising a nucleic acid molecule encoding the VH of the chimeric antigen receptor described herein. In some aspects, the isolated polynucleotide described herein further comprises a nucleic acid molecule encoding the VL of the chimeric antigen receptor. The present disclosure also provides an isolated polynucleotide comprising a nucleic acid molecule encoding VL of the chimeric antigen receptor described herein. In some aspects, the isolated polynucleotide described herein further comprises a nucleic acid molecule encoding the VH of the chimeric antigen receptor.

The present disclosure also provides an isolated polynucleotide comprising a nucleic acid molecule encoding the chimeric antigen receptor described herein.

The present disclosure also provides a vector comprising the isolated polynucleotide disclosed herein. In some aspects, the vector is selected from the group consisting of a retroviral vector, an adenoviral vector, a lentiviral vector, and an adeno-associated viral vector.

The present disclosure also provides an antibody or antigen-binding fragment thereof capable of specifically binding to a TCR VB region, comprising a heavy chain variable region (VH) having an amino acid sequence according to any of SEQ ID NOs: 228-295 and 559-569. In some aspects, the VH comprises an amino acid sequence having at least 85%, at least 90%, at least 91%, at least 92%, at least 93%, at least 94%, at least 95%, at least 96%, at least 97%, at least 98%, at least 99%, or about 100% sequence identity to any of SEQ ID NOs: 228-295 and 559-569.

In some aspects, the antibody or antigen-binding fragment further comprises a light chain variable region (VL) having an amino acid sequence according to any of SEQ ID NOs: 296-369 and 570-583.

The present disclosure also provides an antibody or antigen-binding fragment thereof capable of specifically binding to a TCR VB region, comprising a light chain variable region (VL) having an amino acid sequence according to any of SEQ ID NOs: 296-369 and 570-583. In some aspects described herein, the VL comprises an amino acid sequence having at least 85%, at least 90%, at least 91%, at least 92%, at least 93%, at least 94%, at least 95%, at least 96%, at least 97%, at least 98%, at least 99%, or about 100% sequence identity to any of SEQ ID NOs: 296-369 and 570-583.

In some aspects, the antibody or antigen-binding fragment further comprises a heavy chain variable region (VH) having an amino acid sequence according to any of SEQ ID NOs: 228-295 and 559-569.

In some aspects, the antibody or antigen-binding fragment comprises (i) a variable heavy chain (VH) which comprises: (a) CDR1, CDR2 and CDR3 according to SEQ ID NO:10, SEQ ID NO:11, and SEQ ID NO:12, respectively; or (b) CDR1, CDR2 and CDR3 according to SEQ ID NO:370, SEQ ID NO:371, and SEQ ID NO:372, respectively; or (c) CDR1, CDR2 and CDR3 according to SEQ ID NO:373, SEQ ID NO:374, and SEQ ID NO:375 respectively; or (d) CDR1, CDR2 and CDR3 according to SEQ ID NO:376, SEQ ID NO:377, and SEQ ID NO:378, respectively; or (e) CDR1, CDR2 and CDR3 according to SEQ ID NO:379, SEQ ID NO:380, and SEQ ID NO:381 respectively; or (f) CDR1, CDR2 and CDR3 according to SEQ ID NO:382, SEQ ID NO:383, and SEQ ID NO:384, respectively; or (g) CDR1, CDR2 and CDR3 according to SEQ ID NO:385, SEQ ID NO:386, and SEQ ID NO:387, respectively; or (h) CDR1, CDR2 and CDR3 according to SEQ ID NO:388, SEQ ID NO:389, and SEQ ID NO:390, respectively; or (i) CDR1, CDR2 and CDR3 according to SEQ ID NO:391, SEQ ID NO:392, and SEQ ID NO:393, respectively; or (j) CDR1, CDR2 and CDR3 according to SEQ ID NO:394, SEQ ID NO:395, and SEQ ID NO:396, respectively; or (k) CDR1, CDR2 and CDR3 according to SEQ ID NO:397, SEQ ID NO:398, and SEQ ID NO:399, respectively; or (l) CDR1, CDR2 and CDR3 according to SEQ ID NO:400, SEQ ID NO:401, and SEQ ID NO:402, respectively; or (m) CDR1, CDR2 and CDR3 according to SEQ ID NO:403, SEQ ID NO:404, and SEQ ID NO:405, respectively; or (n) CDR1, CDR2 and CDR3 according to SEQ ID NO:406, SEQ ID NO:407, and SEQ ID NO:408, respectively; or (o) CDR1, CDR2 and CDR3 according to SEQ ID NO:409, SEQ ID NO:410, and SEQ ID NO:411, respectively; or (p) CDR1, CDR2 and CDR3 according to SEQ ID NO:412, SEQ ID NO:413, and SEQ ID NO:414, respectively; or (q) CDR1, CDR2 and CDR3 according to SEQ ID NO:415, SEQ ID NO:416, and SEQ ID NO:417, respectively; or (r) CDR1, CDR2 and CDR3 according to SEQ ID NO:584, SEQ ID NO:585, and SEQ ID NO:586, respectively; or (s) CDR1, CDR2 and CDR3 according to SEQ ID NO:587, SEQ ID NO:588, and SEQ ID NO:589, respectively; or (t) CDR1, CDR2 and CDR3 according to SEQ ID NO:590, SEQ ID NO:591, and SEQ ID NO:592, respectively; or (u) CDR1, CDR2 and CDR3 according to SEQ ID NO:593, SEQ ID NO:594, and SEQ ID NO:595, respectively; and/or (ii) a variable light chain (VL) which comprises: (a) CDR1, CDR2 and CDR3 according to SEQ ID NO:13, SEQ ID NO:14, SEQ ID NO: 15, respectively; or (b) CDR1, CDR2 and CDR3 according to SEQ ID NO:418, SEQ ID NO:419, SEQ ID NO:420, respectively; or (c) CDR1, CDR2 and CDR3 according to SEQ ID NO:421, SEQ ID NO:422, SEQ ID NO:423, respectively; or (d) CDR1, CDR2 and CDR3 according to SEQ ID NO:424, SEQ ID NO:425, SEQ ID NO:426, respectively; or (e) CDR1, CDR2 and CDR3 according to SEQ ID NO:427, SEQ ID NO:428, SEQ ID NO:429, respectively; or (f) CDR1, CDR2 and CDR3 according to SEQ ID NO:430, SEQ ID NO:431, SEQ ID NO:432, respectively; or (g) CDR1, CDR2 and CDR3 according to SEQ ID NO:433, SEQ ID NO:434, SEQ ID NO:435, respectively; or (h) CDR1, CDR2 and CDR3 according to SEQ ID NO:436, SEQ ID NO:437, SEQ ID NO:438, respectively; or (i) CDR1, CDR2 and CDR3 according to SEQ ID NO:439, SEQ ID NO:440, SEQ ID NO:441, respectively; or (j) CDR1, CDR2 and CDR3 according to SEQ ID NO:442, SEQ ID NO:443, SEQ ID NO:444, respectively; or (k) CDR1, CDR2 and CDR3 according to SEQ ID NO:445, SEQ ID NO:446, SEQ ID NO:447, respectively; or (l) CDR1, CDR2 and CDR3 according to SEQ ID NO:448, SEQ ID NO:449, SEQ ID NO:450, respectively; or (m) CDR1, CDR2 and CDR3 according to SEQ ID NO:451, SEQ ID NO:452, SEQ ID NO:453, respectively; or (n) CDR1, CDR2 and CDR3 according to SEQ ID NO:454, SEQ ID NO:455, SEQ ID NO:456, respectively; or (o) CDR1, CDR2 and CDR3 according to SEQ ID NO:457, SEQ ID NO:458, SEQ ID NO:459, respectively; or (p) CDR1, CDR2 and CDR3 according to SEQ ID NO:460, SEQ ID NO:461, SEQ ID NO:462, respectively; or (q) CDR1, CDR2 and CDR3 according to SEQ ID NO:463, SEQ ID NO:464, SEQ ID NO:465, respectively; or (r) CDR1, CDR2 and CDR3 according to SEQ ID NO:596, SEQ ID NO:597, SEQ ID NO:598, respectively; or (s) CDR1, CDR2 and CDR3 according to SEQ ID NO:599, SEQ ID NO:601, SEQ ID NO:601, respectively; or (t) CDR1, CDR2 and CDR3 according to SEQ ID NO:602, SEQ ID NO:603, SEQ ID NO:604, respectively; or (u) CDR1, CDR2 and CDR3 according to SEQ ID NO:605, SEQ ID NO:606, SEQ ID NO:607, respectively, or SEQ ID NO: 608, SEQ ID NO: 609, SEQ ID NO: 610, respectively.

In some aspects described herein, the antibody or antigen-binding fragment thereof is human, humanized, or chimeric. In some aspects, the antibody or antigen-binding fragment thereof is an IgG antibody. In some aspects, the IgG antibody is an IgG1 antibody or an IgG4 antibody. In some aspects, the IgG antibody is an IgG1 antibody. In some aspects, the antibody is an antigen-binding fragment of an antibody. In some aspects, the fragment is selected from the group consisting of Fab, F(ab′)2, Fv, scFv, scFv-Fc, dsFv and a single domain molecule. In some aspects, the fragment is a scFv. In some aspects, the fragment is a Fab. In some aspects, the fragment is an intrabody. In some aspects, the antigen-binding fragment is devoid of an Fc region.

In some aspects described herein, the antibody or antigen-binding fragment described herein, comprises a VH and a VL on the same polypeptide chain. In some aspects described herein, the VH and VL are connected by a linker.

In some aspects of the antibodies or antigen-binding fragment thereof described herein, the TCR Vβ region is selected from the group consisting of Vβ1, Vβ2, Vβ3, Vβ4, Vβ5.1, Vβ5.3, Vβ7.1, Vβ7.2, Vβ8, Vβ9, Vβ11, Vβ12, Vβ13.1, Vβ13.2, Vβ13.6, Vβ14, Vβ16, Vβ17, Vβ18, Vβ20, and Vβ22. In some aspects, the TCR Vβ region is Vβ2.

The present disclosure also provides a method for treating cancer in a subject in need thereof, the method comprising: administering to the subject a therapeutically effective amount of a T-cell composition comprising a CAR, wherein the CAR comprises (i) an antigen binding molecule that specifically binds to a TCR Vβ region, (ii) an extracellular domain, (iii) a transmembrane domain, (iv) a costimulatory domain and (v) an activating domain, wherein the antigen binding molecule is a single chain variable fragment (scFv) comprising: a variable heavy chain according to any one of SEQ ID NOs: 45-131 and 534-544, and/or a variable light chain according to any one of SEQ ID NOs: 132-227 and 545-558.

In some aspects of the methods described herein, the chimeric antigen receptor comprises (i) the variable heavy chain (VH) comprises CDR1, CDR2 and CDR3 according to SEQ ID NO:10, SEQ ID NO:11, and SEQ ID NO:12, respectively, and/or (ii) the variable light chain (VL) comprises CDR1, CDR2 and CDR3 according to SEQ ID NO:13, SEQ ID NO:14, SEQ ID NO:15, respectively; or (i) the variable heavy chain (VH) comprises CDR1, CDR2 and CDR3 according to SEQ ID NO:370, SEQ ID NO:371, and SEQ ID NO:372, respectively, and/or (ii) the variable light chain (VL) comprises CDR1, CDR2 and CDR3 according to SEQ ID NO:418, SEQ ID NO:419, SEQ ID NO:420, respectively; or (i) the variable heavy chain (VH) comprises CDR1, CDR2 and CDR3 according to SEQ ID NO:373, SEQ ID NO:374, and SEQ ID NO:375 respectively, and/or (ii) the variable light chain (VL) comprises CDR1, CDR2 and CDR3 according to SEQ ID NO:421, SEQ ID NO:422, SEQ ID NO:423, respectively; or (i) the variable heavy chain (VH) comprises CDR1, CDR2 and CDR3 according to SEQ ID NO:376, SEQ ID NO:377, and SEQ ID NO:378, respectively, and/or (ii) the variable light chain (VL) comprises CDR1, CDR2 and CDR3 according to SEQ ID NO:424, SEQ ID NO:425, SEQ ID NO:426, respectively; or the variable heavy chain (VH) comprises CDR1, CDR2 and CDR3 according to SEQ ID NO:379, SEQ ID NO:380, and SEQ ID NO:381 respectively, and/or (ii) the variable light chain (VL) comprises CDR1, CDR2 and CDR3 according to SEQ ID NO:427, SEQ ID NO:428, SEQ ID NO:429, respectively; or (i) the variable heavy chain (VH) comprises CDR1, CDR2 and CDR3 according to SEQ ID NO:382, SEQ ID NO:383, and SEQ ID NO:384, respectively, and/or (ii) the variable light chain (VL) comprises CDR1, CDR2 and CDR3 according to SEQ ID NO:430, SEQ ID NO:431, SEQ ID NO:432, respectively; or (i) the variable heavy chain (VH) comprises CDR1, CDR2 and CDR3 according to SEQ ID NO:385, SEQ ID NO:386, and SEQ ID NO:387, respectively, and/or (ii) the variable light chain (VL) comprises CDR1, CDR2 and CDR3 according to SEQ ID NO:433, SEQ ID NO:434, SEQ ID NO:435, respectively; or (i) the variable heavy chain (VH) comprises CDR1, CDR2 and CDR3 according to SEQ ID NO:388, SEQ ID NO:389, and SEQ ID NO:390, respectively, and/or (ii) the variable light chain (VL) comprises CDR1, CDR2 and CDR3 according to SEQ ID NO:436, SEQ ID NO:437, SEQ ID NO:438, respectively; or (i) the variable heavy chain (VH) comprises CDR1, CDR2 and CDR3 according to SEQ ID NO:391, SEQ ID NO:392, and SEQ ID NO:393 respectively, and/or (ii) the variable light chain (VL) comprises CDR1, CDR2 and CDR3 according to SEQ ID NO:439, SEQ ID NO:440, SEQ ID NO:441, respectively; or (i) the variable heavy chain (VH) comprises CDR1, CDR2 and CDR3 according to SEQ ID NO:394, SEQ ID NO:395, and SEQ ID NO:396, respectively, and/or (ii) the variable light chain (VL) comprises CDR1, CDR2 and CDR3 according to SEQ ID NO:442, SEQ ID NO:443, SEQ ID NO:444, respectively; or (i) the variable heavy chain (VH) comprises CDR1, CDR2 and CDR3 according to SEQ ID NO:397, SEQ ID NO:398, and SEQ ID NO:399, respectively, and/or (ii) the variable light chain (VL) comprises CDR1, CDR2 and CDR3 according to SEQ ID NO:445, SEQ ID NO:446, SEQ ID NO:447, respectively; or (i) the variable heavy chain (VH) comprises CDR1, CDR2 and CDR3 according to SEQ ID NO:400, SEQ ID NO:401, and SEQ ID NO:402, respectively, and/or (ii) the variable light chain (VL) comprises CDR1, CDR2 and CDR3 according to SEQ ID NO:448, SEQ ID NO:449, SEQ ID NO:450, respectively; or (i) the variable heavy chain (VH) comprises CDR1, CDR2 and CDR3 according to SEQ ID NO:403, SEQ ID NO:404, and SEQ ID NO:405 respectively, and/or (ii) the variable light chain (VL) comprises CDR1, CDR2 and CDR3 according to SEQ ID NO:451, SEQ ID NO:452, SEQ ID NO:453, respectively; or (i) the variable heavy chain (VH) comprises CDR1, CDR2 and CDR3 according to SEQ ID NO:406, SEQ ID NO:407, and SEQ ID NO:408, respectively, and/or (ii) the variable light chain (VL) comprises CDR1, CDR2 and CDR3 according to SEQ ID NO:454, SEQ ID NO:455, SEQ ID NO:456, respectively; or (i) the variable heavy chain (VH) comprises CDR1, CDR2 and CDR3 according to SEQ ID NO:409, SEQ ID NO:410, and SEQ ID NO:411, respectively, and/or (ii) the variable light chain (VL) comprises CDR1, CDR2 and CDR3 according to SEQ ID NO:457, SEQ ID NO:458, SEQ ID NO:459, respectively; or (i) the variable heavy chain (VH) comprises CDR1, CDR2 and CDR3 according to SEQ ID NO:412, SEQ ID NO:413, and SEQ ID NO:414, respectively, and/or (ii) the variable light chain (VL) comprises CDR1, CDR2 and CDR3 according to SEQ ID NO:460, SEQ ID NO:461, SEQ ID NO:462, respectively; or (i) the variable heavy chain (VH) comprises CDR1, CDR2 and CDR3 according to SEQ ID NO:415, SEQ ID NO:416, and SEQ ID NO:417, respectively, and/or (ii) the variable light chain (VL) comprises CDR1, CDR2 and CDR3 according to SEQ ID NO:463, SEQ ID NO:464, SEQ ID NO:465, respectively; or (i) the variable heavy chain (VH) comprises CDR1, CDR2 and CDR3 according to SEQ ID NO:584, SEQ ID NO:585, and SEQ ID NO:586, respectively, and/or (ii) the variable light chain (VL) comprises CDR1, CDR2 and CDR3 according to SEQ ID NO:596, SEQ ID NO:597, SEQ ID NO:598, respectively; or (i) the variable heavy chain (VH) comprises CDR1, CDR2 and CDR3 according to SEQ ID NO:587, SEQ ID NO:588, and SEQ ID NO:589, respectively, and/or (ii) the variable light chain (VL) comprises CDR1, CDR2 and CDR3 according to SEQ ID NO:599, SEQ ID NO:601, SEQ ID NO:601, respectively; or (i) the variable heavy chain (VH) comprises CDR1, CDR2 and CDR3 according to SEQ ID NO:590, SEQ ID NO:591, and SEQ ID NO:592, respectively, and/or (ii) the variable light chain (VL) comprises CDR1, CDR2 and CDR3 according to SEQ ID NO:602, SEQ ID NO:603, SEQ ID NO:604, respectively; or (i) the variable heavy chain (VH) comprises CDR1, CDR2 and CDR3 according to SEQ ID NO:593, SEQ ID NO:594, and SEQ ID NO:595, respectively, and/or (ii) the variable light chain (VL) comprises CDR1, CDR2 and CDR3 according to SEQ ID NO:605, SEQ ID NO:606, SEQ ID NO:607, respectively, or SEQ ID NO: 608, SEQ ID NO: 609, SEQ ID NO: 610, respectively.

In some aspects, the variable heavy chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NOs: 45-131 and 534-544. In some aspects of the methods described herein, the variable light chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NO: 132-227 and 545-558.

In some aspects of the methods described herein, the cancer is selected from the group consisting of T-cell Lymphoma, T-cell Leukemia, Cutaneous T-cell Lymphoma, Peripheral T-cell Lymphoma (PTCL), Not Otherwise Specified PTCL (PTCL-NOS), Angioimmunoblastic T-cell Lymphoma (AITL), Anaplastic Large-cell Lymphoma (ALCL), Enteropathy-Associated T-cell Lymphoma (EATL), Adult T-cell Leukemia/Lymphoma (ATLL), Hepatosplenic T-cell Lymphoma (HSTL), Subcutaneous Panniculitis-Like T-cell Lymphoma (SPTCL), T-cell Acute Lymphoblastic Leukemia (T-ALL), T-cell Chronic Lymphocytic Leukemia, Large Granular Lymphocyte Leukemia, T-cell Prolymphocytic Leukemia, Lympomatoid Papulosis, Small Medium Pleiomorphic T-cell Lymphoma, Mycosis Fungoides, Sezary Syndrome, and Cytotoxic T-cell Lymphoma.

In some aspects of the methods described herein, the composition is delivered intratumorally, intravenously, intraperitoneally, intramuscularly, intrathecally, transdermally, or subcutaneously. In some aspects, the composition is delivered intravenously or intratumorally.

In some aspects of the methods described herein, the subject is a human.

The present disclosure also provides a method for treating cancer in a subject in need thereof, the method comprising: administering to the subject a therapeutically effective amount of a composition comprising an antibody or antigen-binding fragment thereof capable of specifically binding to a TCR VB region, comprising a (i) heavy chain variable region (VH) having an amino acid sequence according to any of SEQ ID NOs: 228-295 and 559-569, and/or (ii) a light chain variable region (VL) having an amino acid sequence according to any of SEQ ID NOs: 296-369 and 570-583.

In some aspects of the methods described herein, the antibody or antigen-binding fragment comprises (i) a variable heavy chain (VH) which comprises: (a) CDR1, CDR2 and CDR3 according to SEQ ID NO:10, SEQ ID NO:11, and SEQ ID NO:12, respectively; or (b) CDR1, CDR2 and CDR3 according to SEQ ID NO:370, SEQ ID NO:371, and SEQ ID NO:372, respectively; or (c) CDR1, CDR2 and CDR3 according to SEQ ID NO:373, SEQ ID NO:374, and SEQ ID NO:375 respectively; or (d) CDR1, CDR2 and CDR3 according to SEQ ID NO:376, SEQ ID NO:377, and SEQ ID NO:378, respectively; or (e) CDR1, CDR2 and CDR3 according to SEQ ID NO:379, SEQ ID NO:380, and SEQ ID NO:381 respectively; or (f) CDR1, CDR2 and CDR3 according to SEQ ID NO:382, SEQ ID NO:383, and SEQ ID NO:384, respectively; or (g) CDR1, CDR2 and CDR3 according to SEQ ID NO:385, SEQ ID NO:386, and SEQ ID NO:387, respectively; or (h) CDR1, CDR2 and CDR3 according to SEQ ID NO:388, SEQ ID NO:389, and SEQ ID NO:390, respectively; or (i) CDR1, CDR2 and CDR3 according to SEQ ID NO:391, SEQ ID NO:392, and SEQ ID NO:393, respectively; or (j) CDR1, CDR2 and CDR3 according to SEQ ID NO:394, SEQ ID NO:395, and SEQ ID NO:396, respectively; or (k) CDR1, CDR2 and CDR3 according to SEQ ID NO:397, SEQ ID NO:398, and SEQ ID NO:399, respectively; or (l) CDR1, CDR2 and CDR3 according to SEQ ID NO:400, SEQ ID NO:401, and SEQ ID NO:402, respectively; or (m) CDR1, CDR2 and CDR3 according to SEQ ID NO:403, SEQ ID NO:404, and SEQ ID NO:405, respectively; or (n) CDR1, CDR2 and CDR3 according to SEQ ID NO:406, SEQ ID NO:407, and SEQ ID NO:408, respectively; or (o) CDR1, CDR2 and CDR3 according to SEQ ID NO:409, SEQ ID NO:410, and SEQ ID NO:411, respectively; or (p) CDR1, CDR2 and CDR3 according to SEQ ID NO:412, SEQ ID NO:413, and SEQ ID NO:414, respectively; or (q) CDR1, CDR2 and CDR3 according to SEQ ID NO:415, SEQ ID NO:416, and SEQ ID NO:417, respectively; or (r) CDR1, CDR2 and CDR3 according to SEQ ID NO:584, SEQ ID NO:585, and SEQ ID NO:586, respectively; or (s) CDR1, CDR2 and CDR3 according to SEQ ID NO:587, SEQ ID NO:588, and SEQ ID NO:589, respectively; or (t) CDR1, CDR2 and CDR3 according to SEQ ID NO:590, SEQ ID NO:591, and SEQ ID NO:592, respectively; or (u) CDR1, CDR2 and CDR3 according to SEQ ID NO:593, SEQ ID NO:594, and SEQ ID NO:595, respectively; and/or (ii) a variable light chain (VL) which comprises: (a) CDR1, CDR2 and CDR3 according to SEQ ID NO:13, SEQ ID NO:14, SEQ ID NO:15, respectively; or (b) CDR1, CDR2 and CDR3 according to SEQ ID NO:418, SEQ ID NO:419, SEQ ID NO:420, respectively; or (c) CDR1, CDR2 and CDR3 according to SEQ ID NO:421, SEQ ID NO:422, SEQ ID NO:423, respectively; or (d) CDR1, CDR2 and CDR3 according to SEQ ID NO:424, SEQ ID NO:425, SEQ ID NO:426, respectively; or (e) CDR1, CDR2 and CDR3 according to SEQ ID NO:427, SEQ ID NO:428, SEQ ID NO:429, respectively; or (f) CDR1, CDR2 and CDR3 according to SEQ ID NO:430, SEQ ID NO:431, SEQ ID NO:432, respectively; or (g) CDR1, CDR2 and CDR3 according to SEQ ID NO:433, SEQ ID NO:434, SEQ ID NO:435, respectively; or (h) CDR1, CDR2 and CDR3 according to SEQ ID NO:436, SEQ ID NO:437, SEQ ID NO:438, respectively; or (i) CDR1, CDR2 and CDR3 according to SEQ ID NO:439, SEQ ID NO:440, SEQ ID NO:441, respectively; or (j) CDR1, CDR2 and CDR3 according to SEQ ID NO:442, SEQ ID NO:443, SEQ ID NO:444, respectively; or (k) CDR1, CDR2 and CDR3 according to SEQ ID NO:445, SEQ ID NO:446, SEQ ID NO:447, respectively; or (1) CDR1, CDR2 and CDR3 according to SEQ ID NO:448, SEQ ID NO:449, SEQ ID NO:450, respectively; or (m) CDR1, CDR2 and CDR3 according to SEQ ID NO:451, SEQ ID NO:452, SEQ ID NO:453, respectively; or (n) CDR1, CDR2 and CDR3 according to SEQ ID NO:454, SEQ ID NO:455, SEQ ID NO:456, respectively; or (o) CDR1, CDR2 and CDR3 according to SEQ ID NO:457, SEQ ID NO:458, SEQ ID NO:459, respectively; or (p) CDR1, CDR2 and CDR3 according to SEQ ID NO:460, SEQ ID NO:461, SEQ ID NO:462, respectively; or (q) CDR1, CDR2 and CDR3 according to SEQ ID NO:463, SEQ ID NO:464, SEQ ID NO:465, respectively; or (r) CDR1, CDR2 and CDR3 according to SEQ ID NO:596, SEQ ID NO:597, SEQ ID NO:598, respectively; or (s) CDR1, CDR2 and CDR3 according to SEQ ID NO:599, SEQ ID NO:601, SEQ ID NO:601, respectively; or (t) CDR1, CDR2 and CDR3 according to SEQ ID NO:602, SEQ ID NO:603, SEQ ID NO:604, respectively; or (u) CDR1, CDR2 and CDR3 according to SEQ ID NO:605, SEQ ID NO:606, SEQ ID NO:607, respectively, or SEQ ID NO: 608, SEQ ID NO: 609, SEQ ID NO: 610, respectively.

In some aspects of the methods described herein, the variable heavy chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NOs: 228-295 and 559-569. In some aspects, the antibody or antigen-binding fragment further comprises a light chain variable region (VL) having an amino acid sequence according to any of SEQ ID NOs: 296-369 and 570-583. In some aspects of, the variable light chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NO: 296-369 and 570-583. In some aspects of the methods described herein, the antibody or antigen-binding fragment further comprises a heavy chain variable region (VH) having an amino acid sequence according to any of SEQ ID NOs: 228-295 and 559-569.

In some aspects of the methods described herein, the TCR Vβ region is selected from the group consisting of Vβ1, Vβ2, Vβ3, Vβ4, Vβ5.1, Vβ5.3, Vβ7.1, Vβ7.2, Vβ8, Vβ9, Vβ11, Vβ12, Vβ13.1, Vβ13.2, Vβ13.6, Vβ14, Vβ16, Vβ17, Vβ18, Vβ20, and Vβ22.

In some aspects, the TCR Vβ region is Vβ2.

In some aspects of the methods described herein, the composition is delivered intratumorally, intravenously, intraperitoneally, intramuscularly, intrathecally, transdermally, or subcutaneously. In some aspects, the composition is delivered intravenously. In some aspects, the composition is delivered intratumorally.

In some aspects of the methods described herein, the subject is a human.

In some aspects of the methods described herein, the cancer is selected from the group consisting of T-cell Lymphoma, T-cell Leukemia, Cutaneous T-cell Lymphoma, Peripheral T-cell Lymphoma (PTCL), Not Otherwise Specified PTCL (PTCL-NOS), Angioimmunoblastic T-cell Lymphoma (AITL), Anaplastic Large-cell Lymphoma (ALCL), Enteropathy-Associated T-cell Lymphoma (EATL), Adult T-cell Leukemia/Lymphoma (ATLL), Hepatosplenic T-cell Lymphoma (HSTL), Subcutaneous Panniculitis-Like T-cell Lymphoma (SPTCL), T-cell Acute Lymphoblastic Leukemia (T-ALL), T-cell Chronic Lymphocytic Leukemia, Large Granular Lymphocyte Leukemia, T-cell Prolymphocytic Leukemia, Lympomatoid Papulosis, Small Medium Pleiomorphic T-cell Lymphoma, Mycosis Fungoides, Sezary Syndrome, and Cytotoxic T-cell Lymphoma.

The present disclosure also provides a method for treating T-cell mediated diseases in a subject in need thereof, the method comprising: administering to the subject a therapeutically effective amount of a T-cell composition comprising a CAR, wherein the CAR comprises (i) an antigen binding molecule that specifically binds to a TCR Vβ region, (ii) an extracellular domain, (iii) a transmembrane domain, (iv) a costimulatory domain and (v) an activating domain, wherein the antigen binding molecule is a single chain variable fragment (scFv) comprising: a variable heavy chain according to any one of SEQ ID NOs: 45-131 and 534-544, and/or a variable light chain according to any one of SEQ ID NOs: 132-227 and 545-558.

In some aspects of the methods described herein, the chimeric antigen receptor comprises (i) the variable heavy chain (VH) comprises CDR1, CDR2 and CDR3 according to SEQ ID NO:10, SEQ ID NO:11, and SEQ ID NO:12, respectively, or (ii) the variable light chain (VL) comprises CDR1, CDR2 and CDR3 according to SEQ ID NO:13, SEQ ID NO:14, SEQ ID NO:15, respectively; or (i) the variable heavy chain (VH) comprises CDR1, CDR2 and CDR3 according to SEQ ID NO:370, SEQ ID NO:371, and SEQ ID NO:372, respectively, and/or (ii) the variable light chain (VL) comprises CDR1, CDR2 and CDR3 according to SEQ ID NO:418, SEQ ID NO:419, SEQ ID NO:420, respectively; or (i) the variable heavy chain (VH) comprises CDR1, CDR2 and CDR3 according to SEQ ID NO:373, SEQ ID NO:374, and SEQ ID NO:375 respectively, and/or (ii) the variable light chain (VL) comprises CDR1, CDR2 and CDR3 according to SEQ ID NO:421, SEQ ID NO:422, SEQ ID NO:423, respectively; or (i) the variable heavy chain (VH) comprises CDR1, CDR2 and CDR3 according to SEQ ID NO:376, SEQ ID NO:377, and SEQ ID NO:378, respectively, and/or (ii) the variable light chain (VL) comprises CDR1, CDR2 and CDR3 according to SEQ ID NO:424, SEQ ID NO:425, SEQ ID NO:426, respectively; or the variable heavy chain (VH) comprises CDR1, CDR2 and CDR3 according to SEQ ID NO:379, SEQ ID NO:380, and SEQ ID NO:381 respectively, and/or (ii) the variable light chain (VL) comprises CDR1, CDR2 and CDR3 according to SEQ ID NO:427, SEQ ID NO:428, SEQ ID NO:429, respectively; or (i) the variable heavy chain (VH) comprises CDR1, CDR2 and CDR3 according to SEQ ID NO:382, SEQ ID NO:383, and SEQ ID NO:384, respectively, and/or (ii) the variable light chain (VL) comprises CDR1, CDR2 and CDR3 according to SEQ ID NO:430, SEQ ID NO:431, SEQ ID NO:432, respectively; or (i) the variable heavy chain (VH) comprises CDR1, CDR2 and CDR3 according to SEQ ID NO:385, SEQ ID NO:386, and SEQ ID NO:387, respectively, and/or (ii) the variable light chain (VL) comprises CDR1, CDR2 and CDR3 according to SEQ ID NO:433, SEQ ID NO:434, SEQ ID NO:435, respectively; or (i) the variable heavy chain (VH) comprises CDR1, CDR2 and CDR3 according to SEQ ID NO:388, SEQ ID NO:389, and SEQ ID NO:390, respectively, and/or (ii) the variable light chain (VL) comprises CDR1, CDR2 and CDR3 according to SEQ ID NO:436, SEQ ID NO:437, SEQ ID NO:438, respectively; or (i) the variable heavy chain (VH) comprises CDR1, CDR2 and CDR3 according to SEQ ID NO:391, SEQ ID NO:392, and SEQ ID NO:393 respectively, and/or (ii) the variable light chain (VL) comprises CDR1, CDR2 and CDR3 according to SEQ ID NO:439, SEQ ID NO:440, SEQ ID NO:441, respectively; or (i) the variable heavy chain (VH) comprises CDR1, CDR2 and CDR3 according to SEQ ID NO:394, SEQ ID NO:395, and SEQ ID NO:396, respectively, and/or (ii) the variable light chain (VL) comprises CDR1, CDR2 and CDR3 according to SEQ ID NO:442, SEQ ID NO:443, SEQ ID NO:444, respectively; or (i) the variable heavy chain (VH) comprises CDR1, CDR2 and CDR3 according to SEQ ID NO:397, SEQ ID NO:398, and SEQ ID NO:399, respectively, and/or (ii) the variable light chain (VL) comprises CDR1, CDR2 and CDR3 according to SEQ ID NO:445, SEQ ID NO:446, SEQ ID NO:447, respectively; or (i) the variable heavy chain (VH) comprises CDR1, CDR2 and CDR3 according to SEQ ID NO:400, SEQ ID NO:401, and SEQ ID NO:402, respectively, and/or (ii) the variable light chain (VL) comprises CDR1, CDR2 and CDR3 according to SEQ ID NO:448, SEQ ID NO:449, SEQ ID NO:450, respectively; or (i) the variable heavy chain (VH) comprises CDR1, CDR2 and CDR3 according to SEQ ID NO:403, SEQ ID NO:404, and SEQ ID NO:405 respectively, and/or (ii) the variable light chain (VL) comprises CDR1, CDR2 and CDR3 according to SEQ ID NO:451, SEQ ID NO:452, SEQ ID NO:453, respectively; or (i) the variable heavy chain (VH) comprises CDR1, CDR2 and CDR3 according to SEQ ID NO:406, SEQ ID NO:407, and SEQ ID NO:408, respectively, and/or (ii) the variable light chain (VL) comprises CDR1, CDR2 and CDR3 according to SEQ ID NO:454, SEQ ID NO:455, SEQ ID NO:456, respectively; or (i) the variable heavy chain (VH) comprises CDR1, CDR2 and CDR3 according to SEQ ID NO:409, SEQ ID NO:410, and SEQ ID NO:411, respectively, and/or (ii) the variable light chain (VL) comprises CDR1, CDR2 and CDR3 according to SEQ ID NO:457, SEQ ID NO:458, SEQ ID NO:459, respectively; or (i) the variable heavy chain (VH) comprises CDR1, CDR2 and CDR3 according to SEQ ID NO:412, SEQ ID NO:413, and SEQ ID NO:414, respectively, and/or (ii) the variable light chain (VL) comprises CDR1, CDR2 and CDR3 according to SEQ ID NO:460, SEQ ID NO:461, SEQ ID NO:462, respectively; or (i) the variable heavy chain (VH) comprises CDR1, CDR2 and CDR3 according to SEQ ID NO:415, SEQ ID NO:416, and SEQ ID NO:417, respectively, and/or (ii) the variable light chain (VL) comprises CDR1, CDR2 and CDR3 according to SEQ ID NO:463, SEQ ID NO:464, SEQ ID NO:465, respectively; or (i) the variable heavy chain (VH) comprises CDR1, CDR2 and CDR3 according to SEQ ID NO:584, SEQ ID NO:585, and SEQ ID NO:586, respectively, and/or (ii) the variable light chain (VL) comprises CDR1, CDR2 and CDR3 according to SEQ ID NO:596, SEQ ID NO:597, SEQ ID NO:598, respectively; or (i) the variable heavy chain (VH) comprises CDR1, CDR2 and CDR3 according to SEQ ID NO:587, SEQ ID NO:588, and SEQ ID NO:589, respectively, and/or (ii) the variable light chain (VL) comprises CDR1, CDR2 and CDR3 according to SEQ ID NO:599, SEQ ID NO:601, SEQ ID NO:601, respectively; or (i) the variable heavy chain (VH) comprises CDR1, CDR2 and CDR3 according to SEQ ID NO:590, SEQ ID NO:591, and SEQ ID NO:592, respectively, and/or (ii) the variable light chain (VL) comprises CDR1, CDR2 and CDR3 according to SEQ ID NO:602, SEQ ID NO:603, SEQ ID NO:604, respectively; or (i) the variable heavy chain (VH) comprises CDR1, CDR2 and CDR3 according to SEQ ID NO:593, SEQ ID NO:594, and SEQ ID NO:595, respectively, and/or (ii) the variable light chain (VL) comprises CDR1, CDR2 and CDR3 according to SEQ ID NO:605, SEQ ID NO:606, SEQ ID NO:607, respectively, or SEQ ID NO: 608, SEQ ID NO: 609, SEQ ID NO: 610, respectively.

In some aspects, the variable heavy chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NOs: 45-131 and 534-544. In some aspects, the variable light chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NO: 132-227 and 545-558.

In some aspects of the methods described herein, the T-cell mediated disease is selected from the group consisting of Atherosclerosis, Parkinson's disease, Alzheimer's disease, Type 1 Diabetes Mellitus, Grave's Disease, Hashimoto's Thyroiditis, Addison's Disease, Rheumatoid Arthritis (RA), Multiple Sclerosis (MS), Psoriasis, Psoriatic Arthritis, Lichen Planus, Lichen Planopilaris, Morphea, Scleroderma, Systemic Sclerosis, acute or chronic Graft-Versus-Host Disease (GVHD), Cardiac Organ Transplant Rejection, Pulmonary Organ Transplant Rejection, Renal Organ Transplant Rejection, Interstitial Lung Disease, Scleroderma, Alopecia Areata, Vitiligo, Celiac Disease (Sprue), Myasthenia Gravis, Pernicious Anemia, Sjogren's Syndrome, Systemic Lupus Erythematosus, Chronic Inflammatory Demyelinating Polyneuropathy, Guillain-Barre Syndrome, Inflammatory Bowel Disease, Crohn's Disease, and Ulcerative Colitis.

In some aspects of the methods described herein, the composition is delivered intratumorally, intravenously, intraperitoneally, intramuscularly, intrathecally, transdermally, or subcutaneously. In some aspects, the composition is delivered intravenously.

In some aspects of the methods described herein, the subject is a human.

The present disclosure also provides a method for treating T-cell mediated diseases in a subject in need thereof, the method comprising: administering to the subject a therapeutically effective amount of a composition comprising an antibody or antigen-binding fragment thereof, capable of specifically binding to a TCR VB region, comprising a (i) heavy chain variable region (VH) having an amino acid sequence according to any of SEQ ID NOs: 228-295 and 559-569, and/or (ii) a light chain variable region (VL) having an amino acid sequence according to any of SEQ ID NOs: 296-369 and 570-583.

In some aspects of the methods described herein, the antibody or antigen-binding fragment comprises (i) a variable heavy chain (VH) which comprises: (a) CDR1, CDR2 and CDR3 according to SEQ ID NO:10, SEQ ID NO:11, and SEQ ID NO:12, respectively; or (b) CDR1, CDR2 and CDR3 according to SEQ ID NO:370, SEQ ID NO:371, and SEQ ID NO:372, respectively; or (c) CDR1, CDR2 and CDR3 according to SEQ ID NO:373, SEQ ID NO:374, and SEQ ID NO:375 respectively; or (d) CDR1, CDR2 and CDR3 according to SEQ ID NO:376, SEQ ID NO:377, and SEQ ID NO:378, respectively; or (e) CDR1, CDR2 and CDR3 according to SEQ ID NO:379, SEQ ID NO:380, and SEQ ID NO:381 respectively; or (f) CDR1, CDR2 and CDR3 according to SEQ ID NO:382, SEQ ID NO:383, and SEQ ID NO:384, respectively; or (g) CDR1, CDR2 and CDR3 according to SEQ ID NO:385, SEQ ID NO:386, and SEQ ID NO:387, respectively; or (h) CDR1, CDR2 and CDR3 according to SEQ ID NO:388, SEQ ID NO:389, and SEQ ID NO:390, respectively; or (i) CDR1, CDR2 and CDR3 according to SEQ ID NO:391, SEQ ID NO:392, and SEQ ID NO:393, respectively; or (j) CDR1, CDR2 and CDR3 according to SEQ ID NO:394, SEQ ID NO:395, and SEQ ID NO:396, respectively; or (k) CDR1, CDR2 and CDR3 according to SEQ ID NO:397, SEQ ID NO:398, and SEQ ID NO:399, respectively; or (l) CDR1, CDR2 and CDR3 according to SEQ ID NO:400, SEQ ID NO:401, and SEQ ID NO:402, respectively; or (m) CDR1, CDR2 and CDR3 according to SEQ ID NO:403, SEQ ID NO:404, and SEQ ID NO:405, respectively; or (n) CDR1, CDR2 and CDR3 according to SEQ ID NO:406, SEQ ID NO:407, and SEQ ID NO:408, respectively; or (o) CDR1, CDR2 and CDR3 according to SEQ ID NO:409, SEQ ID NO:410, and SEQ ID NO:411, respectively; or (p) CDR1, CDR2 and CDR3 according to SEQ ID NO:412, SEQ ID NO:413, and SEQ ID NO:414, respectively; or (q) CDR1, CDR2 and CDR3 according to SEQ ID NO:415, SEQ ID NO:416, and SEQ ID NO:417, respectively; or (r) CDR1, CDR2 and CDR3 according to SEQ ID NO:584, SEQ ID NO:585, and SEQ ID NO:586, respectively; or (s) CDR1, CDR2 and CDR3 according to SEQ ID NO:587, SEQ ID NO:588, and SEQ ID NO:589, respectively; or (t) CDR1, CDR2 and CDR3 according to SEQ ID NO:590, SEQ ID NO:591, and SEQ ID NO:592, respectively; or (u) CDR1, CDR2 and CDR3 according to SEQ ID NO:593, SEQ ID NO:594, and SEQ ID NO:595, respectively; and/or (ii) a variable light chain (VL) which comprises: (a) CDR1, CDR2 and CDR3 according to SEQ ID NO:13, SEQ ID NO:14, SEQ ID NO:15, respectively; or (b) CDR1, CDR2 and CDR3 according to SEQ ID NO:418, SEQ ID NO:419, SEQ ID NO:420, respectively; or (c) CDR1, CDR2 and CDR3 according to SEQ ID NO:421, SEQ ID NO:422, SEQ ID NO:423, respectively; or (d) CDR1, CDR2 and CDR3 according to SEQ ID NO:424, SEQ ID NO:425, SEQ ID NO:426, respectively; or (e) CDR1, CDR2 and CDR3 according to SEQ ID NO:427, SEQ ID NO:428, SEQ ID NO:429, respectively; or (f) CDR1, CDR2 and CDR3 according to SEQ ID NO:430, SEQ ID NO:431, SEQ ID NO:432, respectively; or (g) CDR1, CDR2 and CDR3 according to SEQ ID NO:433, SEQ ID NO:434, SEQ ID NO:435, respectively; or (h) CDR1, CDR2 and CDR3 according to SEQ ID NO:436, SEQ ID NO:437, SEQ ID NO:438, respectively; or (i) CDR1, CDR2 and CDR3 according to SEQ ID NO:439, SEQ ID NO:440, SEQ ID NO:441, respectively; or (j) CDR1, CDR2 and CDR3 according to SEQ ID NO:442, SEQ ID NO:443, SEQ ID NO:444, respectively; or (k) CDR1, CDR2 and CDR3 according to SEQ ID NO:445, SEQ ID NO:446, SEQ ID NO:447, respectively; or (1) CDR1, CDR2 and CDR3 according to SEQ ID NO:448, SEQ ID NO:449, SEQ ID NO:450, respectively; or (m) CDR1, CDR2 and CDR3 according to SEQ ID NO:451, SEQ ID NO:452, SEQ ID NO:453, respectively; or (n) CDR1, CDR2 and CDR3 according to SEQ ID NO:454, SEQ ID NO:455, SEQ ID NO:456, respectively; or (o) CDR1, CDR2 and CDR3 according to SEQ ID NO:457, SEQ ID NO:458, SEQ ID NO:459, respectively; or (p) CDR1, CDR2 and CDR3 according to SEQ ID NO:460, SEQ ID NO:461, SEQ ID NO:462, respectively; or (q) CDR1, CDR2 and CDR3 according to SEQ ID NO:463, SEQ ID NO:464, SEQ ID NO:465, respectively; or (r) CDR1, CDR2 and CDR3 according to SEQ ID NO:596, SEQ ID NO:597, SEQ ID NO:598, respectively; or (s) CDR1, CDR2 and CDR3 according to SEQ ID NO:599, SEQ ID NO:601, SEQ ID NO:601, respectively; or (t) CDR1, CDR2 and CDR3 according to SEQ ID NO:602, SEQ ID NO:603, SEQ ID NO:604, respectively; or (u) CDR1, CDR2 and CDR3 according to SEQ ID NO:605, SEQ ID NO:606, SEQ ID NO:607, respectively, or SEQ ID NO: 608, SEQ ID NO: 609, SEQ ID NO: 610, respectively.

In some aspects, the variable heavy chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NOs: 228-295 and 559-569. In some aspects, the antibody or antigen-binding fragment further comprises a light chain variable region (VL) having an amino acid sequence according to any of SEQ ID NOs: 296-369 and 570-583.

In some aspects, the variable light chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NO: 296-369 and 570-583.

In some aspects, the antibody or antigen-binding fragment further comprises a heavy chain variable region (VH) having an amino acid sequence according to any of SEQ ID NOs: 228-295 and 559-569.

In some aspects of the methods described herein, the TCR Vβ region is selected from the group consisting of Vβ1, Vβ2, Vβ3, Vβ4, Vβ5.1, Vβ5.3, Vβ7.1, Vβ7.2, Vβ8, Vβ9, Vβ11, Vβ12, Vβ13.1, Vβ13.2, Vβ13.6, Vβ14, Vβ16, Vβ17, Vβ18, Vβ20, and Vβ22. In some aspects, the TCR Vβ region is Vβ2.

In some aspects of the methods described herein, the T-cell mediated disease is selected from the group consisting of Atherosclerosis, Parkinson's disease, Alzheimer's disease, Type 1 Diabetes Mellitus, Grave's Disease, Hashimoto's Thyroiditis, Addison's Disease, Rheumatoid Arthritis (RA), Multiple Sclerosis (MS), Psoriasis, Psoriatic Arthritis, Lichen Planus, Lichen Planopilaris, Morphea, Scleroderma, Systemic Sclerosis, acute or chronic Graft-Versus-Host Disease (GVHD), Cardiac Organ Transplant Rejection, Pulmonary Organ Transplant Rejection, Renal Organ Transplant Rejection, Interstitial Lung Disease, Scleroderma, Alopecia Areata, Vitiligo, Celiac Disease (Sprue), Myasthenia Gravis, Pernicious Anemia, Sjogren's Syndrome, Systemic Lupus Erythematosus, Chronic Inflammatory Demyelinating Polyneuropathy, Guillain-Barre Syndrome, Inflammatory Bowel Disease, Crohn's Disease, and Ulcerative Colitis.

In some aspects of the methods described herein, the composition is delivered intratumorally, intravenously, intraperitoneally, intramuscularly, intrathecally or subcutaneous delivery.

In some aspects, the composition is delivered intravenously.

In some aspects of the methods described herein, the subject is a human.

The present disclosure also provides a use of (a) the CAR described herein, or (b) the antibody or antigen binding fragment of described herein, for the manufacture of a medicament for treating a T-cell mediated disease. In some aspects, the T-cell mediated disease is selected from the group consisting of Atherosclerosis, Parkinson's disease, Alzheimer's disease, Type 1 Diabetes Mellitus, Grave's Disease, Hashimoto's Thyroiditis, Addison's Disease, Rheumatoid Arthritis (RA), Multiple Sclerosis (MS), Psoriasis, Psoriatic Arthritis, Lichen Planus, Lichen Planopilaris, Morphea, Scleroderma, Systemic Sclerosis, acute or chronic Graft-Versus-Host Disease (GVHD), Cardiac Organ Transplant Rejection, Pulmonary Organ Transplant Rejection, Renal Organ Transplant Rejection, Interstitial Lung Disease, Scleroderma, Alopecia Areata, Vitiligo, Celiac Disease (Sprue), Myasthenia Gravis, Pernicious Anemia, Sjogren's Syndrome, Systemic Lupus Erythematosus, Chronic Inflammatory Demyelinating Polyneuropathy, Guillain-Barre Syndrome, Inflammatory Bowel Disease, Crohn's Disease, and Ulcerative Colitis.

The present disclosure also provides a use of (a) the CAR described herein, or (b) the antibody or antigen binding fragment of decribed herein, for the manufacture of a medicament for treating a cancer. In some aspects, the cancer is selected from the group consisting of T-cell Lymphoma, T-cell Leukemia, Cutaneous T-cell Lymphoma, Peripheral T-cell Lymphoma (PTCL), Not Otherwise Specified PTCL (PTCL-NOS), Angioimmunoblastic T-cell Lymphoma (AITL), Anaplastic Large-cell Lymphoma (ALCL), Enteropathy-Associated T-cell Lymphoma (EATL), Adult T-cell Leukemia/Lymphoma (ATLL), Hepatosplenic T-cell Lymphoma (HSTL), Subcutaneous Panniculitis-Like T-cell Lymphoma (SPTCL), T-cell Acute Lymphoblastic Leukemia (T-ALL), T-cell Chronic Lymphocytic Leukemia, Large Granular Lymphocyte Leukemia, T-cell Prolymphocytic Leukemia, Lympomatoid Papulosis, Small Medium Pleiomorphic T-cell Lymphoma, Mycosis Fungoides, Sezary Syndrome, and Cytotoxic T-cell Lymphoma.

The present disclosure also provides a kit of parts comprising at least one of a (i) variable heavy chain, and/or (ii) variable light chain of (a) the CAR disclosed herein, or (b) the antibody or antigen binding fragment disclosed herein. In some aspects, the kit further comprises instructions for use.

The present disclosure also provides an adenoassociated virus (AAV) vector encoding a chimeric antigen receptor (CAR) comprising (i) an antigen binding molecule that specifically binds to a TCR Vβ region, (ii) an extracellular domain, (iii) a transmembrane domain, (iv) a costimulatory domain and (v) an activating domain wherein the antigen binding molecule is a single chain variable fragment (scFv) comprising: a variable heavy chain according to any one of SEQ ID NOs: 45-131 and 534-544, or a variable light chain according to any one of SEQ ID NOs: 132-227 and 545-558.

In some aspects of the AAV vector described herein (i) the variable heavy chain (VH) comprises CDR1, CDR2 and CDR3 according to SEQ ID NO:10, SEQ ID NO:11, and SEQ ID NO:12, respectively, and/or (ii) the variable light chain (VL) comprises CDR1, CDR2 and CDR3 according to SEQ ID NO:13, SEQ ID NO:14, SEQ ID NO:15, respectively; or (i) the variable heavy chain (VH) comprises CDR1, CDR2 and CDR3 according to SEQ ID NO:370, SEQ ID NO:371, and SEQ ID NO:372, respectively, and/or (ii) the variable light chain (VL) comprises CDR1, CDR2 and CDR3 according to SEQ ID NO:418, SEQ ID NO:419, SEQ ID NO:420, respectively; or (i) the variable heavy chain (VH) comprises CDR1, CDR2 and CDR3 according to SEQ ID NO:373, SEQ ID NO:374, and SEQ ID NO:375 respectively, and/or (ii) the variable light chain (VL) comprises CDR1, CDR2 and CDR3 according to SEQ ID NO:421, SEQ ID NO:422, SEQ ID NO:423, respectively; or (i) the variable heavy chain (VH) comprises CDR1, CDR2 and CDR3 according to SEQ ID NO:376, SEQ ID NO:377, and SEQ ID NO:378, respectively, and/or (ii) the variable light chain (VL) comprises CDR1, CDR2 and CDR3 according to SEQ ID NO:424, SEQ ID NO:425, SEQ ID NO:426, respectively; or the variable heavy chain (VH) comprises CDR1, CDR2 and CDR3 according to SEQ ID NO:379, SEQ ID NO:380, and SEQ ID NO:381 respectively, and/or (ii) the variable light chain (VL) comprises CDR1, CDR2 and CDR3 according to SEQ ID NO:427, SEQ ID NO:428, SEQ ID NO:429, respectively; or (i) the variable heavy chain (VH) comprises CDR1, CDR2 and CDR3 according to SEQ ID NO:382, SEQ ID NO:383, and SEQ ID NO:384, respectively, and/or (ii) the variable light chain (VL) comprises CDR1, CDR2 and CDR3 according to SEQ ID NO:430, SEQ ID NO:431, SEQ ID NO:432, respectively; or (i) the variable heavy chain (VH) comprises CDR1, CDR2 and CDR3 according to SEQ ID NO:385, SEQ ID NO:386, and SEQ ID NO:387, respectively, and/or (ii) the variable light chain (VL) comprises CDR1, CDR2 and CDR3 according to SEQ ID NO:433, SEQ ID NO:434, SEQ ID NO:435, respectively; or (i) the variable heavy chain (VH) comprises CDR1, CDR2 and CDR3 according to SEQ ID NO:388, SEQ ID NO:389, and SEQ ID NO:390, respectively, and/or (ii) the variable light chain (VL) comprises CDR1, CDR2 and CDR3 according to SEQ ID NO:436, SEQ ID NO:437, SEQ ID NO:438, respectively; or (i) the variable heavy chain (VH) comprises CDR1, CDR2 and CDR3 according to SEQ ID NO:391, SEQ ID NO:392, and SEQ ID NO:393 respectively, and/or (ii) the variable light chain (VL) comprises CDR1, CDR2 and CDR3 according to SEQ ID NO:439, SEQ ID NO:440, SEQ ID NO:441, respectively; or (i) the variable heavy chain (VH) comprises CDR1, CDR2 and CDR3 according to SEQ ID NO:394, SEQ ID NO:395, and SEQ ID NO:396, respectively, and/or (ii) the variable light chain (VL) comprises CDR1, CDR2 and CDR3 according to SEQ ID NO:442, SEQ ID NO:443, SEQ ID NO:444, respectively; or (i) the variable heavy chain (VH) comprises CDR1, CDR2 and CDR3 according to SEQ ID NO:397, SEQ ID NO:398, and SEQ ID NO:399, respectively, and/or (ii) the variable light chain (VL) comprises CDR1, CDR2 and CDR3 according to SEQ ID NO:445, SEQ ID NO:446, SEQ ID NO:447, respectively; or (i) the variable heavy chain (VH) comprises CDR1, CDR2 and CDR3 according to SEQ ID NO:400, SEQ ID NO:401, and SEQ ID NO:402, respectively, and/or (ii) the variable light chain (VL) comprises CDR1, CDR2 and CDR3 according to SEQ ID NO:448, SEQ ID NO:449, SEQ ID NO:450, respectively; or (i) the variable heavy chain (VH) comprises CDR1, CDR2 and CDR3 according to SEQ ID NO:403, SEQ ID NO:404, and SEQ ID NO:405 respectively, and/or (ii) the variable light chain (VL) comprises CDR1, CDR2 and CDR3 according to SEQ ID NO:451, SEQ ID NO:452, SEQ ID NO:453, respectively; or (i) the variable heavy chain (VH) comprises CDR1, CDR2 and CDR3 according to SEQ ID NO:406, SEQ ID NO:407, and SEQ ID NO:408, respectively, and/or (ii) the variable light chain (VL) comprises CDR1, CDR2 and CDR3 according to SEQ ID NO:454, SEQ ID NO:455, SEQ ID NO:456, respectively; or (i) the variable heavy chain (VH) comprises CDR1, CDR2 and CDR3 according to SEQ ID NO:409, SEQ ID NO:410, and SEQ ID NO:411, respectively, and/or (ii) the variable light chain (VL) comprises CDR1, CDR2 and CDR3 according to SEQ ID NO:457, SEQ ID NO:458, SEQ ID NO:459, respectively; or (i) the variable heavy chain (VH) comprises CDR1, CDR2 and CDR3 according to SEQ ID NO:412, SEQ ID NO:413, and SEQ ID NO:414, respectively, and/or (ii) the variable light chain (VL) comprises CDR1, CDR2 and CDR3 according to SEQ ID NO:460, SEQ ID NO:461, SEQ ID NO:462, respectively; or (i) the variable heavy chain (VH) comprises CDR1, CDR2 and CDR3 according to SEQ ID NO:415, SEQ ID NO:416, and SEQ ID NO:417, respectively, and/or (ii) the variable light chain (VL) comprises CDR1, CDR2 and CDR3 according to SEQ ID NO:463, SEQ ID NO:464, SEQ ID NO:465, respectively; or (i) the variable heavy chain (VH) comprises CDR1, CDR2 and CDR3 according to SEQ ID NO:584, SEQ ID NO:585, and SEQ ID NO:586, respectively, and/or (ii) the variable light chain (VL) comprises CDR1, CDR2 and CDR3 according to SEQ ID NO:596, SEQ ID NO:597, SEQ ID NO:598, respectively; or (i) the variable heavy chain (VH) comprises CDR1, CDR2 and CDR3 according to SEQ ID NO:587, SEQ ID NO:588, and SEQ ID NO:589, respectively, and/or (ii) the variable light chain (VL) comprises CDR1, CDR2 and CDR3 according to SEQ ID NO:599, SEQ ID NO:601, SEQ ID NO:601, respectively; or (i) the variable heavy chain (VH) comprises CDR1, CDR2 and CDR3 according to SEQ ID NO:590, SEQ ID NO:591, and SEQ ID NO:592, respectively, and/or (ii) the variable light chain (VL) comprises CDR1, CDR2 and CDR3 according to SEQ ID NO:602, SEQ ID NO:603, SEQ ID NO:604, respectively; or (i) the variable heavy chain (VH) comprises CDR1, CDR2 and CDR3 according to SEQ ID NO:593, SEQ ID NO:594, and SEQ ID NO:595, respectively, and/or (ii) the variable light chain (VL) comprises CDR1, CDR2 and CDR3 according to SEQ ID NO:605, SEQ ID NO:606, SEQ ID NO:607, respectively, or SEQ ID NO: 608, SEQ ID NO: 609, SEQ ID NO: 610, respectively.

In some aspects, the variable heavy chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NOs: 45-131 and 534-544. In some aspects, the variable light chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NO: 132-227 and 545-558.

In some aspects of the AAV described herein, the TCR Vβ region is selected from the group consisting of Vβ1, Vβ2, Vβ3, Vβ4, Vβ5.1, Vβ5.3, Vβ7.1, Vβ7.2, Vβ8, Vβ9, Vβ11, Vβ12, Vβ13.1, Vβ13.2, Vβ13.6, Vβ14, Vβ16, Vβ17, Vβ18, Vβ20, and Vβ22. In some aspects, the TCR Vβ region is Vβ2.

In some aspects of the AAV described herein, the antigen-binding domain is a scFv. In some aspects, the scFv comprises an amino acid sequence according to any of SEQ ID NOs: 14-44.

In some aspects of the AAV described herein, the costimulatory domain comprises a 4-1BB costimulatory domain. In some aspects, the 4-1BB costimulatory domain comprises an amino acid sequence according to SEQ ID NO: 8.

In some aspects of the AAV described herein, the transmembrane domain comprises an hCD28 transmembrane. In some aspects, the transmembrane domain comprises a hCD28 amino acid sequence according to SEQ ID NO: 7.

In some aspects of the AAV described herein, the signaling domain comprises a CD247 signaling domain. In some aspects, the signaling domain comprises a CD247 signaling domain amino acid sequence according to SEQ ID NO: 9.

In some aspects of the AAV described herein, the scFv comprises a CD28 signal peptide. In some aspects, the scFv comprises a CD28 signal peptide an amino acid sequence according to SEQ ID NO:3.

In some aspects of the AAV described herein, the scFv comprises a linker peptide. In some aspects, the scFv comprises a linker peptide according to SEQ ID NO: 4.

The present disclosure also provides methods which further comprise determining the subject's TCR Vβ usage. In some aspects, determining the subject's TCR Vβ usage comprises obtaining a sample from the subject, wherein the sample is a blood sample or a tissue sample. In some aspects, the subject is administered (a) a CAR described herein, or (b) the antibody or antigen binding fragment described herein, that corresponds to the subject's TCR Vβ usage.

In some aspects of the antibody described herein, the ADCC activity of the antibody composition is increased or decreased by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 1-fold, about 2-fold, about 3-fold, or about 4-fold, or increased or decreased by about 5% to about 400%.

In some aspects of the antibody described herein, the antigen or the antigen binding fragment has an increased antibody-dependent cell mediated cytotoxicity activity achieved through afucosylation.

In some aspects of the antibody described herein, the antibody or antigen binding fragment has an increased antibody-dependent cell mediated cytotoxicity activity achieved through glycosylation.

### DESCRIPTION OF FIGURES

FIG. 1 is a chimeric antigen receptor expressing lentiviral vector plasmid. The lentiviral vector plasmid comprises an EF-1a core promoter, an hCD28 signal peptide, a variable heavy chain, a linker, a variable light chain, an hCD8a hinge, an hCD28 transmembrane, an h4-1bb signal peptide, an hCD247 signal peptide, a P2A peptide, and EGFP.

FIG. 2A is a chart quantified by flow cytometry showing the effective killing of Vβ2+ but not Vβ13+ cutaneous T-cell lymphoma (CTCL) cells by mCAR-Vβ2 T-cells without knockout (KO) of the endogenous T-cell receptor alpha constant (TRAC) region.

FIG. 2B is a chart quantified by flow cytometry showing the effective killing of Vβ2+ but not Vβ13+ CTCL cells by mCAR-Vβ2 T-cells with KO of the endogenous TRAC region.

FIGS. 2C-2D are charts quantified by flow cytometry which show the specific targeting/killing of Vβ2+ CTCL cells while sparing Vβ2-negative normal T-cells and non-T-cells in two different patient samples.

FIG. 2E is a chart quantified by flow cytometry showing the killing specificity of mCAR-Vβ2 TRAC KO effector T-cells in a peripheral blood mononuclear cells (PBMC) cultured healthy control.

FIG. 2F is a chart quantified by flow cytometry showing the killing specificity of mCAR-Vβ2 TRAC KO effector T-cells in a purified pan-T cultured healthy control.

FIG. 3 is a schematic of a mouse model used to assess in vivo Vβ2+ T lymphoma killing by allogeneic TCR-KO mCAR-Vβ2 T-cells from a healthy donor.

FIGS. 4A-4C are charts quantified by flow cytometry which show the CTCL count in spleen, bone marrow and blood samples, respectively, between no CAR treated control (NC) and mCAR-Vβ2 treated patient derived CTCLs engrafted immunodeficient NSG mice.

FIGS. 5A-5C are charts quantified by flow cytometry which show the Vβ2-negative CD4+ T-cell count in spleen, bone marrow and blood samples, respectively, between no CAR treated control (NC) and mCAR-Vβ2 treated patient derived CTCLs engrafted immunodeficient NSG mice.

FIGS. 6A-6C are charts quantified by flow cytometry which show the mCAR-Vβ2 count in spleen, bone marrow and blood samples, respectively, between no CAR treated control (NC) and mCAR-Vβ2 treated patient derived CTCLs engrafted immunodeficient NSG mice.

FIG. 7 is a chart quantified by flow cytometry which shows the Jurkat-Vβ2 counts from bone marrow, between no CAR treated control and mCAR-Vβ2 treated Jurkat-Vβ2 cells engrafted immunodeficient NSG mice after 10 days.

FIG. 8 is a chart quantified by flow cytometry which shows the mCAR-Vβ2 count from bone marrow, between no CAR treated control and mCAR-Vβ2 treated Jurkat-Vβ2 cells engrafted immunodeficient NSG mice after 10 days.

FIGS. 9A-9D are charts quantified by flow cytometry which show the effective killing of Vβ2+ CTCL cells by humanized CAR-Vβ2 T-cells with KO of the endogenous TRAC region.

FIG. 10A is a chart quantified by single cell RNA sequencing and T-cell receptor (TCR) sequencing to identify and quantify T-cell clones of CTCL patients and healthy controls.

FIG. 10B is a chart quantified by anti-VP antibody staining and flow cytometry which shows the usage frequency of various Vβ by the malignant cells across CTCL patients.

FIG. 11 is a chart quantified by flow cytometry which shows the efficiency of TCR alpha constant region knock out (TRAC KO) in mCAR-VP T-cells and the purity of TRAC KO mCAR-Vβ2 T-cells after magnetic bead removal of remaining CD3+ cells.

FIG. 12 shows the proportions of CD4+ and CD8+ T-cells during CAR-cell in vitro expansion (center) and the lentiviral transduction efficiency in each subtype (left CD4+, right CD8+) as determined by flow cytometry.

FIG. 13A shows the reduction in MHC class I protein expression by mCAR Vβ2 T-cells following knock out of beta-2-microglobulin (B2M KO) compared with a no knock-out control (NC).

FIG. 13B shows the reduction in MHC class II protein expression by mCAR-Vβ2 T-cells following knock out of the class II major histocompatibility complex transactivator (CIITA KO) compared with a no knock-out control (NC).

FIGS. 14A-14B are charts quantified by flow cytometry which show the Vβ2+ CTCL count in bone marrow and spleen samples, respectively, between no CAR treated control (NC), CAR-CD19 and hCAR-Vβ2 treated patient derived CTCL cells engrafted immunodeficient NSG mice.

FIGS. 15A-15B are charts quantified by flow cytometry which show the Vβ2-negative T cell count in bone marrow and spleen samples, respectively, between no CAR treated control (NC), CAR-CD19 and hCAR-Vβ2 treated patient derived CTCL cells engrafted immunodeficient NSG mice.

FIGS. 16A-16B are charts quantified by flow cytometry which show the CD8+ CAR count in bone marrow and spleen samples, respectively, between no CAR treated control (NC), CAR-CD19 and hCAR-Vβ2 treated patient derived CTCL cells engrafted immunodeficient NSG mice.

FIGS. 17A-B are charts quantified by flow cytometry which show the CD4+ CAR count in bone marrow and spleen samples, respectively, between no CAR treated control (NC), CAR-CD19 and hCAR-Vβ2 treated patient derived CTCL cells engrafted immunodeficient NSG mice.

FIG. 18 a chimeric antigen receptor expressing Adeno-associated virus (AAV) vector plasmid. The AAV vector plasmid comprises a left homology arm (LHA) of human TRAC region, T2A in-frame to TRAC coding sequence, a CAR-Vb2, a P2A peptide, EGFP, a bGH poly A signal, and a right homology arm (RHA) of human TRAC region.

FIG. 19A is a chart quantified by flow cytometry which shows CD3 and HLA-A/B/C expression on pan-T cells post TRAC/B2M/CIITA triple KO and AAV-CAR delivery before and after CD3+ cell depletion using CD3+ microbeads.

FIG. 19B is a chart quantified by flow cytometry which shows post TRAC/B2M/CIITA triple KO and AAV-CAR-Vβ2 transduction, expression of GFP as CAR expressing reporter on CD3-HLA-A/B/C-T-cells, two (left) and ten (right) days post transduction.

FIG. 20 is a chart quantified by flow cytometry which shows the effective killing of CTCL cells by mCAR Vβ2 via TRAC/B2M/CIITA triple-KO and AAV-CAR strategy.

FIG. 21 is a chart that provides a reference array for identifying each of the CAR constructs presented in the charts in each of FIGS. 22-33.

FIG. 22 shows the results of the immunogenicity of various allogeneic CAR constructs, according to the alignment presented per FIG. 21. This figure is quantified by flow cytometry detecting anti-allogeneic cell IgM and assays the serum from healthy control 1 (HC1).

FIG. 23 shows the results of the immunogenicity of various allogeneic CAR constructs, according to the alignment presented per FIG. 21. This figure is quantified by flow cytometry detecting anti-allogeneic cell IgM and assays the serum from healthy control 2 (HC2).

FIG. 24 shows the results of the immunogenicity of various allogeneic CAR constructs, according to the alignment presented per FIG. 21. This figure is quantified by flow cytometry detecting anti-allogeneic cell IgM and assays the serum from healthy control 3 (HC3).

FIG. 25 shows the results of the immunogenicity of various allogeneic CAR constructs, according to the alignment presented per FIG. 21. This figure is quantified by flow cytometry detecting anti-allogeneic cell IgM and assays the serum from a Vβ1 patient.

FIG. 26 shows the results of the immunogenicity of various allogeneic CAR constructs, according to the alignment presented per FIG. 21. This figure is quantified by flow cytometry detecting anti-allogeneic cell IgM and assays the serum from a Vβ2 patient.

FIG. 27 shows the results of the immunogenicity of various allogeneic CAR constructs, according to the alignment presented per FIG. 21. This figure is quantified by flow cytometry detecting anti-allogeneic cell IgM and assays the serum from a Vβ13.2 patient.

FIG. 28 shows the results of the immunogenicity of various allogeneic CAR constructs, according to the alignment presented per FIG. 21. This figure is quantified by flow cytometry detecting anti-allogeneic cell IgG and assays the serum from healthy control 1 (HC1).

FIG. 29 shows the results of the immunogenicity of various allogeneic CAR constructs, according to the alignment presented per FIG. 21. This figure is quantified by flow cytometry detecting anti-allogeneic cell IgG and assays the serum from healthy control 2 (HC2).

FIG. 30 shows the results of the immunogenicity of various allogeneic CAR constructs, according to the alignment presented per FIG. 21. This figure is quantified by flow cytometry detecting anti-allogeneic cell IgG and assays the serum from healthy control 3 (HC3).

FIG. 31 shows the results of the immunogenicity of various allogeneic CAR constructs, according to the alignment presented per FIG. 21. This figure is quantified by flow cytometry detecting anti-allogeneic cell IgG and assays the serum from a Vβ1 patient.

FIG. 32 shows the results of the immunogenicity of various allogeneic CAR constructs, according to the alignment presented per FIG. 21. This figure is quantified by flow cytometry detecting anti-allogeneic cell IgG and assays the serum from a Vβ2 patient.

FIG. 33 shows the results of the immunogenicity of various allogeneic CAR constructs, according to the alignment presented per FIG. 21. This figure is quantified by flow cytometry detecting anti-allogeneic cell IgG and assays the serum from a Vβ13.2 patient.

FIG. 34A shows the results of in vivo imaging system (IVIS) imaging in immunodeficient NSG mice which have not been treated (NC), mice that have been administered Jurkat-Vβ2+ cells, mice that have been administered Jurkat-Vβ2+ cells expressing luciferase, and mice that have been administered Jurkat-Vβ2+ cells expressing luciferase plus hCAR-Vβ2 T cells, in a ventral position.

FIG. 34B is a bar chart which shows the results of Luciferin signaling from the region of interest (ROI) via IVIS imaging in immunodeficient NSG mice which have not been treated (NC), mice that have been administered Jurkat-Vβ2+ cells, mice that have been administered Jurkat-Vβ2+ cells expressing luciferase, and mice that have been administered Jurkat-Vβ2+ cells expressing luciferase plus hCAR-Vβ2 T cells.

FIG. 35A shows the results of in vivo imaging system (IVIS) imaging in immunodeficient NSG mice which have not been treated (NC), mice that have been administered Jurkat-Vβ2+ cells, mice that have been administered Jurkat-Vβ2+ cells expressing luciferase, and mice that have been administered Jurkat-Vβ2+ cells expressing luciferase plus hCAR-Vβ2 T cells, in a dorsal position.

FIG. 35B is a bar chart which shows the results of Luciferin signaling from the region of interest (ROI) via IVIS imaging in immunodeficient NSG mice which have not been treated (NC), mice that have been administered Jurkat-Vβ2+ cells, mice that have been administered Jurkat-Vβ2+ cells expressing luciferase, and mice that have been administered Jurkat-Vβ2+ cells expressing luciferase plus hCAR-Vβ2 T cells.

FIG. 36A shows the CD3− purity, CD4, and CD8 population percentages, and CAR expression of AAV-dependent allogeneic hCAR-Vβ2 T cells (see bottom row) in comparison to lentiviral-dependent allogeneic CAR-CD19 T cells (see top row), as quantified by flow cytometry.

FIG. 36B are charts quantified by flow cytometry that show the effective killing of Vβ2+ cutaneous T-Cell lympohoma (CTCL) cells by AAV hCAR-Vβ2 T-cells, in comparison to CAR-CD19 in cells obtained from Vβ2+ patient 1 (see left chart) and Vβ2+ patient 1 (see right chart).

FIG. 36C are charts quantified by flow cytometry that show the live Jurkat-TRBV20-1 (Vβ2+, see left chart) or Jurkat-TRBV6-2 (Vβ13.2+, see right chart) cell counts following an overnight in-vitro culture with allogeneic lenti-CAR-CD19 T cells or AAV-hCAR-Vβ2 T cells, at various E:T ratios.

FIG. 37 are charts quantified by flow cytometry that show the CD3− purity, CD4 and CD8 population percentages and expression of green florescence protein (GFP), as a chimeric antigen receptor (CAR) reporter on lentiviral delivered-CAR-CD19 (see top row), lenti-hCAR-Vβ2 (see middle row), and AAV-hCAR-Vβ2 T cells (see bottom row).

FIGS. 38A-38G show the results of the total CD4 T cells isolated from a Vβ2+ PTCL patient, which were adoptively transferred into groups of NSG mice, that were then treated with an allogeneic triple-KO AAV-hCAR-Vβ2 (AAV hCAR-Vβ2) or lenti-CAR-CD19 (CAR-CD19), which were generated from healthy donor pan T cells in comparison to an optionally, no-treatment control (NC). Following the three day post-treatment, Vβ2+ CTCL cells (FIG. 38A), Vβ2− normal T cells (FIG. 38B), CD69+% in CD8 CAR-T cells (FIG. 38C), and CD69+% in CD4 CAR-T cells (FIG. 38D), all from spleen samples were used to quantify results via flow cytometry. Additionally, Vβ2+ CTCL cells (FIG. 38E), CD69+% in CD8 CAR-T cells (FIG. 38F), and CD69+% in CD4 CAR-T cells (FIG. 37G), all from bone marrow (BM) were used to quantify results via flow cytometry.

FIGS. 39A-B show the long-term monitoring of Jurkat-TRBV20-1-lucifer cell-bearing NSG mice via IVIS (luciferin bioluminescence signals) across the time period of 2, 10, 16, 23, and 30 days between mice without treatment (NC) (see top row), following a single treatment with CAR-CD19 T cells (see top middle row), lenti-hCAR-Vβ2 T cells (see bottom middle row), and AAV-hCAR-Vβ2 T cells (see bottom row), via the ventral (FIG. 39A) and dorsal (FIG. 39B) positions, respectively.

FIGS. 39C-39D are charts which show the biolumisence of Jurkat-TRBV20-1-lucifer cell-bearing NSG mice via IVIS (luciferin bioluminescence signals), between 0 and 30 days among mice without treatment (NC), mice treated with CAR-CD19 T cells, Lenti-hCAR-Vβ2 T cells, and AAV-hCAR-Vβ T cells, in the ventral (FIG. 39C) and dorsal (FIG. 39D) positions.

FIGS. 40A-40B show the body weight (FIG. 40A) and percentage of survival (FIG. 40B) of the same NSG mice as FIGS. 39A-39D, bearing Jurkat-TRBV20-1-lucifer cells, without treatment (NC), following treatment with CAR-CD19 T cells, lenti-hCAR-Vβ2 T cells, and AAV-hCAR-Vβ2 T cells.

FIG. 41 is an exemplerary schematic of the different cytokine combinations used to expand hCAR-Vβ2 T cells in vitro.

FIG. 42 is a chart which shows the live CD3− hCAR-Vβ2 T cell counts at day 0, day 7 and day 12, under different cytokine combination conditions.

FIGS. 43A-43B are charts which show the live Jurkat-TRBV20-1 cell counts (FIG. 43A) and live CD3− hCAR-Vβ2 T cell counts (FIG. 43B) between 0 and 4 days post culture, from the repeated Jurkat-TRBV20-1 killing assay (see FIG. 41), as quantified by trypan blue cell counting and flow cytometry.

FIGS. 44A-44C are charts which show CD45RA+CD45RO+% (FIG. 44A) and Tim3 (FIG. 44B) mean fluorescence intensity (MFI) of CD8+CD3− hCAR-Vβ2 T cells expression, as quantified by flow cytometry. FIG. 44C is a chart which shows the IFNγ+% of 7-day cytokine expanded CD8+CD3− hCAR-Vβ2 T cells after 6-hour Jurkat-TRBV20-1 stimulation in vitro, as quantified by flow cytometry.

FIG. 45A is a chart which shows the IFNγ+% of 12-day cytokine expanded resting CD8+CD3− hCAR-Vβ2 T cells, quantified by flow cytometry.

FIG. 45B is a chart which shows the CD45RA+CD45RO+% of 7-day and 12-day cytokine expanded resting CD8+CD3− hCAR-Vβ2 T cells, as quantified by flow cytometry.

FIG. 46 is an exemplerary depiction of the optimal cytokine combinations promoting CAR-T expansion in vitro.

FIG. 47 shows charts which illustrate the expression of Lens Culinaris Agglutinin (LCA) on the surface of parental expiCHO antibody producing cells (see left chart), pre-sorted expiCHO cells post Fut8 knockout (KO) (see middle chart), and post-sorted (purified) expiCHO cells post Fut8 KO (see right chart), as quantified by flow cytometry.

FIG. 48 is a chart which shows an anti-Vβ2 antibody competition assay between 1.25βg/ml mouseand humanized anti-Vβ2-FITC antibody as quantified by flow cytometry.

FIG. 49 is a chart which shows the functional antibody-dependent cellular cytotoxicity (ADCC) assay of humanized anti-Vβ2 antibody and mouse anti-Vβ2 antibody.

FIG. 50 is a chart which depicts the live Jurkat-TRBV20-1 target cell count, following an overnight co-culture with effector NK cells from a healthy donor at different E:T ratios without an antibody addition, 100 ng/ml of mouse anti-Vβ2 antibody, and humanized anti-Vβ2 antibody, as determined by flow cytometry.

FIGS. 51A-51B are charts which show the results of live CTCL cell counts as determined by flow cytometry of PBMC, from Vβ2+ CTCL patient 1 (FIG. 51A), or from Vβ2+ CTCL patient 2 (FIG. 51B), following an overnight culture with NK effector cells from a healthy donor at different E:T ratios, without an antibody addition, without the addition of mouse anti-Vβ2 antibody, and with humanized anti-Vβ2 antibody.

FIGS. 52A-52B are charts which show the results of live Vβ2-negative normal T cell counts, as quantified by flow cytometry, from the the PBMC of Vβ2+ CTCL patient 1 (FIG. 52A) or from Vβ2+ CTCL patient 2 (FIG. 52B), which were cultured overnight with natural killer (NK) effector cells from a healthy donor at different E:T ratios without antibody addition, mouse anti-Vβ2 antibody, and humanized anti-Vβ2 antibody.

FIGS. 53A-53B are charts which show the results of live non-T cell counts, as quantified by flow cytometry, from the PBMC of Vβ2+ CTCL patient 1 (FIG. 52A) or from Vβ2+ CTCL patient 2 (FIG. 52B), which were cultured overnight with NK effector cells from a healthy donor at different E:T ratios without antibody addition, with addition of mouse anti-Vβ2 antibody, and humanized anti-Vβ2 antibody.

FIG. 54 are images which show the successful treatment of T cell lymphoma via an anti-Vβ2 humanized therapeutic antibody treatment in NSG mice across the time period of 7, 14, and 16 days between control antibody and NK cells (see top row), followed by anti-Vβ2 antibody and NK cells (see middle row), and solely anti-Vβ2 antibody administration (see bottom row).

FIG. 55A is a graph which shows the specificity of anti-Vβ1 CAR to Vβ1 over Vβ3, Vβ7.1, and no Vβ-expressing Jurkat effector cells.

FIG. 55B is a table that provides % area under “CD69 activation” for anti-Vβ1 CAR to Vβ1 over Vβ3, Vβ7.1, and no Vβ-expressing Jurkat effector cells.

FIG. 56A is a graph which shows the specificity of anti-Vβ7.2 CAR to Vβ7.2 over Vβ13.1, Vβ13.6, and no Vβ-expressing Jurkat effector cells.

FIG. 56B is a table that provides % area under “CD69 activation” for anti-Vβ7.2 CAR to Vβ7.2 over Vβ13.1, Vβ13.6, and no Vβ-expressing Jurkat effector cells.

FIG. 57A is a graph which shows the specificity of anti-Vβ13.6 CAR to Vβ13.6 over Vβ13.1, Vβ17, and no Vβ-expressing Jurkat effector cells.

FIG. 57B is a table that provides % area under “CD69 activation” for anti-Vβ13.6 CAR to Vβ13.6 over Vβ13.1, Vβ17, and no Vβ-expressing Jurkat effector cells.

### DETAILED DESCRIPTION

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. As described herein, the term “a” or “an” means “single.” As disclosed herein, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

As disclosed herein, the language “comprising” constitutes otherwise analogous aspects disclosed in the terms “consisting of” and/or “consisting essentially of”.

Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Thus, any value recited herein is inclusive of a precise value, as well as values which are about the same as the precise value. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “effective amount” or “pharmaceutically effective amount” or “therapeutically effective amount” as used herein refers to the amount or quantity of a therapeutic described herein which is sufficient to elicit the required or desired therapeutic response, or in other words, the amount which is sufficient to elicit an appreciable biological response when administered to a patient.

The term “unit dosage form” or “unit dose composition” as used herein refers to a device containing a quantity of a therapeutic described herein, said quantity being such that one or more predetermined units may be provided as a single therapeutic administration.

As used herein, the term “administration” refers to the administration to a subject. Administration to an animal subject (e.g., to a human) can be by any appropriate route. “Administering” refers to the physical introduction of a composition comprising a therapeutic agent into a subject, using any of the various methods and delivery systems known to those skilled in the art. In some aspects of the present disclosure, administration can be enteral administration, including oral and aboral administration, administration via endoscopic delivery system, administration via a nasogastric tube, administration via a nasojejunal tube, administration via esophagogastroduodenoscopy, administration via colonoscopy, or administration via retention enema, via parenteral administration (e.g., intramuscular, subdermal, subcutaneous, intravenous, and intradermal injection), or administration via any other suitable route of administration known to the skilled in the art, or any combination thereof. Administering can also be performed, for example, once, a plurality of times, and/or over one or more extended periods.

As used herein, the terms “treat,” “treated,” and “treating” mean both therapeutic and prophylactic treatment or preventative measures wherein the object is to reverse, alleviate, ameliorate, lessen, inhibit, slow down progression, development, severity or recurrence of an undesired symptom, complication, condition, biochemical indicia of a disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. In some instances, treatment includes prolonging survival as compared to expected survival if not receiving treatment.

A “complete response” or “complete remission” is defined for any given cancer type as the absence of cancer cells detectable by imaging or molecular methods conventionally used for detection of that type of cancer. A “complete response” does not necessarily mean that all cancer cells are absent from the patient. For cancers in which multiple conventional imaging or molecular methods are conventionally used for detection, the absence of detectable cancer cells using any one of such multiple methods is sufficient to indicate a “complete response” for purposes of the present disclosure.

A “partial response” or “partial remission” is defined for any given cancer type as at least a 50% reduction in estimated number of cancer cells or tumor burden detectable by imaging or molecular methods conventionally used for detection of that type of cancer. For cancers in which multiple conventional imaging or molecular methods are conventionally used for detection, a 50% reduction of detectable cancer cells using any one of such multiple methods is sufficient to indicate a “partial response” for purposes of the present disclosure.

A “poor response” is any response to a CAR or antibody or antigen binding fragment, or of the composition of the disclosure, treatment that is not a “complete response” or a “partial response.” A poor response can include an increase in cancer cells or tumor burden as detectable using conventional imaging or molecular methods for detection of that type of cancer. A poor response can also include a minimal decrease in cancer cells that is still not sufficient to be considered “partial remission.”

As used herein, “CAR” or “CAR-T” toxicity is an early response to CAR or antibody or antigen binding fragment treatment, and includes cytokine release syndrome and neurotoxicity. Although CAR-T toxicity is often considered an adverse reaction, it results from T cell activity and, thus, is also an indicator of likely efficacy of the CAR T cell therapy.

As used herein, “Cytokine release syndrome” or “CRS” is characterized by high fever, myalgias, malaise, respiratory insufficiency, hemodynamic instability and capillary leak with hypotension, tachycardia, hypoxia, tachypnea, hemophagocytic lymphohistiocytosis/macrophage activation syndrome, or other organ toxicity associated with elevated serum cytokine concentrations. Elevated cytokines and associated molecules include interferon (IFN)-γ, IL-2, soluble IL-2Ra, IL-6, soluble IL-6R, granulocyte-macrophage colony-stimulating factor (GM-CSF), and other cytokines primarily secreted by the monocytes and/or macrophages such as IL-1, IL-6, IL-8, IL-10, IL-12, tumor necrosis factor (TNF)-α, IFN-α, monocyte chemotactic protein (MCP)-1, macrophage inflammatory protein (MIP) 1α. CRS usually occurs within a few days of administration of the genetically modified T cells to the patient.

As used herein, “Neurotoxicity” associated with CAR therapy is characterized by encephalopathy, headache, delirium, anxiety, tremor, aphasia, decreased level of consciousness, confusion, seizures, or cerebral edema. Neurotoxicity can be associated with elevated serum concentrations of IL-6, IFN-γ, and TNF-α.

As used herein, the term “amelioration” or “ameliorating” refers to a lessening of severity of at least one indicator of a condition or disease. As used herein, the term “preventing” or “prevention” refers to delaying or forestalling the onset, development or progression of a condition or disease for a period of time, including weeks, months, or years. As used herein, the term “prophylactic” (e.g., “prophylactic agent”, “prophylactic treatment”, “prophylactically effective amount”), refers to any complete or partial prevention of a disease or symptom thereof and/or can be therapeutic in terms of a partial or complete cure for a disease and/or adverse effect and/or symptom attributable to the disease.

A “disease” is a state of health of an animal or subject wherein the subject cannot maintain homeostasis (e.g., cancer and autoimmune diseases, T cell mediated diseases, etc . . . ), and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

“Cancer” as used herein can encompass all types of oncogenic processes and/or cancerous growths. In this disclosure, cancer can include, but is not limited to primary tumors as well as metastatic tissues or malignantly transformed cells, tissues, or organs. Cancer can encompasse histopathologies and stages, e.g., stages of invasiveness/severity, of a cancer. Cancer can include relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors.

Exemplary T-cell-associated cancer or T-cell malignancies include but are not limited to T-cell Lymphoma, T-cell Leukemia, Cutaneous T-cell Lymphoma, Peripheral T-cell Lymphoma (PTCL), Not Otherwise Specified PTCL (PTCL-NOS), Angioimmunoblastic T-cell Lymphoma (AITL), Anaplastic Large-cell Lymphoma (ALCL), Enteropathy-Associated T-cell Lymphoma (EATL), Adult T-cell Leukemia/Lymphoma (ATLL), Hepatosplenic T-cell Lymphoma (HSTL), Subcutaneous Panniculitis-Like T-cell Lymphoma (SPTCL), T-cell Acute Lymphoblastic Leukemia (T-ALL), T-cell Chronic Lymphocytic Leukemia, Large Granular Lymphocyte Leukemia, T-cell Prolymphocytic Leukemia, Lympomatoid Papulosis, Small Medium Pleiomorphic T-cell Lymphoma, Mycosis Fungoides, Sezary Syndrome, and Cytotoxic T-cell Lymphoma.

The term “autoimmune” in relation to TCR means that such TCR is involved in the development of an autoimmune disease.

The term “autoimmune disease” for example, as used herein is defined as a disorder that results from an autoimmune response. An autoimmune disease can result from an inappropriate and excessive response to a self-antigen. Exemplary autoimmune diseases that can be treated include, but are not limited to, Type 1 Diabetes Mellitus, Grave's Disease, Hashimoto's Thyroiditis, Addison's Disease, Rheumatoid Arthritis (RA), Multiple Sclerosis (MS), Psoriasis, Psoriatic Arthritis, Lichen Planus, Lichen Planopilaris, Morphea, Scleroderma, Systemic Sclerosis, acute or chronic Graft-Versus-Host Disease (GVHD), Cardiac Organ Transplant Rejection, Pulmonary Organ Transplant Rejection, Renal Organ Transplant Rejection, Interstitial Lung Disease, Stiff Man Syndrome, Scleroderma, Alopecia Areata, Vitiligo, Celiac Disease (Sprue), Myasthenia Gravis, Pernicious Anemia, Sjogren's Syndrome, Systemic Lupus Erythematosus, Chronic Inflammatory Demyelinating Polyneuropathy, Guillain-Barre Syndrome, Inflammatory Bowel Disease, Crohn's Disease, and Ulcerative Colitis. In addition, there are other inflammatory disorders that may not historically have been considered autoimmune, but where reactive T cells are nonetheless drivers of inflammation that is fundamental to the disease pathogenesis. Indeed, chronic inflammatory diseases have been recognized as the most significant cause of death in the world today, with more than 50% of all deaths being attributable to inflammation-related diseases such as ischemic heart disease, stroke, cancer, diabetes mellitus, chronic kidney disease, non-alcoholic fatty liver disease (NAFLD) and autoimmune and neurodegenerative conditions [GBD 2017 Causes of Death Collaborators. Global, regional, and national age-sex-specific mortality for 282 causes of death in 195 countries and territories, 1980-2017: a systematic analysis for the Global Burden of Disease Study 2017. Lancet 392, 1736-1788 (2018).]. These include atherosclerosis and resultant cardiovascular disease, neurodegenerative disorders such as Parkinson's disease and Altzheimer's disease, chronic obstructive pulmonary disease, which are encompassed within the term autoimmune disease.

As used herein “plasma cells” refer to a type of white blood cells which can produce and secrete antibodies. Plasma cells are also referred to as plasmocytes, plasmacytes, or effector B cells.

As used herein, the terms “therapeutically effective amount”, “therapeutically effective”, “effective amount” or “in an effective amount” are used interchangeably herein and refer to the amount of a compound, preparation, substance or composition that is effective to achieve a specific biological result as described herein, such as but not limited to treating or reducing the count of a cancer or tumor, or to lessen or alleviate a autoimmune disease.

As used herein, the terms “subject” and “patient” are used interchangeably. The subject can be an animal. The subject can be a mammal such as a non-human animal (e.g., cow, pig, horse, cat, dog, rat, mouse, monkey or other primate, etc.). The subject can be a human.

The term “cytotoxicity” as used in this specification, refers to an unintended or undesirable alteration in the normal state of a cell. The normal state of a cell can refer to a state that is manifested or exists prior to the cell's exposure to a cytotoxic composition, agent and/or condition. Generally, a cell that is in a normal state is one that is in homeostasis. An unintended or undesirable alteration in the normal state of a cell can be manifested in the form of, for example, cell death (e.g., programmed cell death), a decrease in replicative potential, a decrease in cellular integrity such as membrane integrity, a decrease in metabolic activity, a decrease in developmental capability, or any of the cytotoxic effects disclosed in the present application.

The phrase “reducing cytotoxicity” or “reduce cytotoxicity” refers to a reduction in degree or frequency of unintended or undesirable alterations in the normal state of a cell upon exposure to a cytotoxic composition, agent and/or condition. The phrase can refer to reducing the degree of cytotoxicity in an individual cell that is exposed to a cytotoxic composition, agent and/or condition, or to reducing the number of cells of a population that exhibit cytotoxicity when the population of cells is exposed to a cytotoxic composition, agent and/or condition.

The term “disrupting” and its grammatical equivalents as used herein can refer to a process of altering a gene, e.g., by deletion, insertion, mutation, rearrangement, or any combination thereof. For example, a gene can be disrupted by “knockout” (KO). Disrupting a gene can be partially reducing or completely suppressing expression of the gene. Disrupting a gene can also cause activation of a different gene, for example, a downstream gene. As described herein, exemplary KO of the endogenous TCR in T cells strongly ablated alloreactivity in comparison to TCR-expressing T cells (Stenger, D., et al., Blood 136(12):1407-1418, Sep., 2020).

As used herein, the terms “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, the term “conservative sequence modifications” is intended to refer to amino acid modifications that do not significantly affect or alter the binding characteristics of the antibody or CAR containing the amino acid sequence. Such conservative modifications include amino acid substitutions, additions and deletions. Modifications can be introduced into an antibody of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, for example, one or more amino acid residues within the extracellular regions of the CAR of the disclosure can be replaced with other amino acid residues having a similar side chain or charge and the altered CAR can be tested for the ability to bind their targets using the functional assays described herein.

The term “variant” refers to a polypeptide that has a substantially identical amino acid sequence to a reference amino acid sequence, or is encoded by a substantially identical nucleotide sequence. In aspects described herein, the variant can be a functional variant. The term “functional variant” refers to a polypeptide that has a substantially identical amino acid sequence to a reference amino acid sequence, or is encoded by a substantially identical nucleotide sequence, and is capable of having one or more activities of the reference amino acid sequence.

A polypeptide, antibody, polynucleotide, vector, cell, or composition which is “isolated” refers to a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature.

As disclosed herein, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

“Percent identity” refers to the extent of identity between two sequences (e.g., amino acid sequences or nucleic acid sequences). Percent identity can be determined by aligning two sequences, introducing gaps to maximize identity between the sequences. Alignments can be generated using programs known in the art. For non-limiting purposes herein, alignment of nucleotide sequences can be performed with the blastn program set at default parameters, and alignment of amino acid sequences can be performed with the blastp program set at default parameters (see National Center for Biotechnology Information (NCBI) on the worldwide web, ncbi.nlm.nih.gov).

Human “T cell receptor”, also referred to as “TCR”, “T receptor”, is a heterodimeric protein complex found on the surface of a T lymphocyte. The T receptor is present only on T lymphocytes. The main function of TCR is to specifically recognize processed antigens bound to the molecules of major histocompatibility complex (HLA). Human TCR comprises two subunits, α and β chains, or γ and δ chains, connected through a disulfide bond and docked onto the cell membrane. Each of the TCR chains has an N-terminal variable (V) domain, a connecting domain, and a constant (C) domain connected to a transmembrane domain that anchors the receptor in the T lymphocyte plasma membrane. The T receptor reacts with the MHC/antigen complex via six regions determining complementarity thereof (CDRs): three alpha chain regions and three beta-chain regions.

A small fraction of T lymphocytes has the γ/δ type T receptors. They are arranged similar to the α/β receptors, but differ in their primary structure and have a number of functional features. They exhibit a much lower variability (limited clone specificity), they recognize antigens in the complex with “non-classical” (non-MHC) antigen-presenting molecules or even free antigens.

As used herein, the terms “T cell receptor beta variable chain,” “TCRVβ,” “TCRVb,” and “TCRβV” are used interchangeably to refer to an extracellular region of the T cell receptor beta chain which comprises the antigen recognition domain of the T cell receptor. The term “Vβ,” “Vβ,” “variable beta,” “variable β,” and “v beta” and any grammatical equivalents are interchangeably used to refer to the variable beta chain of a T cell receptor. The term TCRVβ or TCRβV includes isoforms, mammalian, e.g., human TCRβV, species homologs of human and analogs comprising at least one common epitope with TCRβV. Isoforms of TCRVβ can be identified by integers or letters of the alphabet, e.g., 1, 2, 3, 4 or A, B, C. Human TCRβV comprises a gene family comprising subfamilies including, but not limited to: a TCRβ V1 subfamily, a TCRβ V2 subfamily, a TCRβ V3 subfamily, a TCRβ V4 subfamily, a TCRβ V5 subfamily, a TCRβ V6 subfamily, a TCRβ V7 subfamily, a TCRβ V8 subfamily, a TCRβ V9 subfamily, a TCRβ V10 subfamily, a TCRβ V11 subfamily, a TCRβ V12 subfamily, a TCRβ V13 subfamily, a TCRβ V14 subfamily, a TCRβ V15 subfamily, a TCRβ V16 subfamily, a TCRβ V17 subfamily, a TCRβ V18 subfamily, a TCRβ V19 subfamily, a TCRβ V20 subfamily, a TCRβ V21 subfamily, a TCRβ V22 subfamily, a TCRβ V23 subfamily, TCRβ V24 subfamily, or a TCRβ V25 subfamily. An exemplary list of TCRVβ and subfamilies is provided in Table 1 (below).

TABLE 1
TCRVβ Subfamily

| IMGT | IMGT | IMGT | Accession |
|---|---|---|---|
| Subgroup | Gene name | Allele name | Number |
| TRBV1 | TRBV1 | TRBV1*01 | L36092 |
| TRBV2 | TRBV2 | TRBV2*01 | L36092 |
| | | TRBV2*02 | M62379 |
| | | TRBV2*03 | M64351 |
| TRBV3 | TRBV3-1 | TRBV3-1*01 | U07977 |
| | | TRBV3-1*02 | L06889 |
| | TRBV3-2 | TRBV3-2*01 | L36092 |
| | | TRBV3-2*02 | U07978 |
| | | TRBV3-2*03 | M33240 |
| TRBV4 | TRBV4-1 | TRBV4-1*01 | U07977 |
| | | TRBV4-1*02 | M13855 |
| | TRBV4-2 | TRBV4-2*01 | U07975 |
| | | TRBV4-2*02 | X58811 |
| | TRBV4-3 | TRBV4-3*01 | U07978 |
| | | TRBV4-3*02 | X58812 |
| | | TRBV4-3*03 | L06888 |
| | | TRBV4-3*04 | X57616 |
| TRBV5 | TRBV5-1 | TRBV5-1*01 | L36092 |
| | | TRBV5-1*02 | M14271 |
| | TRBV5-2 | TRBV5-2*01 | L36092 |
| | TRBV5-3 | TRBV5-3*01 | X61439 |
| | | TRBV5-3*02 | X61439 |
| | TRBV5-4 | TRBV5-4*01 | AF009660 |
| | | TRBV5-4*02 | L36092 |
| | | TRBV5-4*03 | X57615 |
| | | TRBV5-4*04 | S50547 |
| | TRBV5-5 | TRBV5-5*01 | X58804 |
| | | TRBV5-5*02 | L36092 |
| | | TRBV5-5*03 | X57611 |
| | TRBV5-6 | TRBV5-6*01 | X58801 |
| | TRBV5-7 | TRBV5-7*01 | L36092 |
| | TRBV5-8 | TRBV5-8*01 | L36092 |
| | | TRBV5-8*02 | X58803 |
| TRBV6 | TRBV6-1 | TRBV6-1*01 | X58803 |
| | TRBV6-2 | TRBV6-2*01 | X61445 |
| | TRBV6-3 | TRBV6-3*01 | U07978 |
| | TRBV6-4 | TRBV6-4*01 | X61653 |
| | | TRBV6-4*02 | AF009660 |
| | TRBV6-5 | TRBV6-5*01 | L36092 |
| | TRBV6-6 | TRBV6-6*01 | L36092 |
| | | TRBV6-6*02 | AF009662 |
| | | TRBV6-6*03 | X58815 |
| | | TRBV6-6*04 | X74848 |
| | | TRBV6-6*05 | L06892 |
| | TRBV6-7 | TRBV6-7*01 | L36092 |
| | TRBV6-8 | TRBV6-8*01 | L36092 |
| | TRBV6-9 | TRBV6-9*01 | X61447 |
| TRBV7 | TRBV7-1 | TRBV7-1*01 | X61444 |
| | TRBV7-2 | TRBV7-2*01 | X61442 |
| | | TRBV7-2*02 | L36190 |
| | | TRBV7-2*03 | U07975 |
| | | TRBV7-2*04 | M27387 |
| | TRBV7-3 | TRBV7-3*01 | X61440 |
| | | TRBV7-3*02 | M97943 |
| | | TRBV7-3*03 | AF009660 |
| | | TRBV7-3*04 | X74843 |
| | | TRBV7-3*05 | M13550 |
| | TRBV7-4 | TRBV7-4*01 | L36092 |
| | | TRBV7-4*02 | L13762 |
| | TRBV7-5 | TRBV7-5*01 | L36092 |
| | | TRBV7-5*02 | AF009663 |
| | TRBV7-6 | TRBV7-6*01 | L36092 |
| | | TRBV7-6*02 | X58806 |
| | TRBV7-7 | TRBV7-7*01 | L36092 |
| | | TRBV7-7*02 | X57607 |
| | TRBV7-8 | TRBV7-8*01 | M11953 |
| | | TRBV7-8*02 | X61441 |
| | | TRBV7-8*03 | M27384 |
| | TRBV7-9 | TRBV7-9*01 | L36092 |
| | | TRBV7-9*02 | M15564 |
| | | TRBV7-9*03 | AF009663 |
| | | TRBV7-9*04 | M14261 |
| | | TRBV7-9*05 | M27385 |
| | | TRBV7-9*06 | X74844 |
| | | TRBV7-9*07 | L14854 |
| TRBV8 | TRBV8-1 | TRBV8-1*01 | L36092 |
| | | TRBV8-1*02 | IMGT000021 |
| | TRBV8-2 | TRBV8-2*01 | L36092 |
| | | TRBV8-2*02 | IMGT000021 |
| TRBV9 | TRBV9 | TRBV9*01 | L36092 |
| | | TRBV9*02 | AF009660 |
| | | TRBV9*03 | M27380 |
| TRBV10 | TRBV10-1 | TRBV10-1*01 | L36092 |
| | | TRBV10-1*02 | AF009660 |
| | | TRBV10-1*03 | U17051 |
| | TRBV10-2 | TRBV10-2*01 | L36092 |
| | | TRBV10-2*02 | IMGT000021 |
| | TRBV10-3 | TRBV10-3*01 | U03115 |
| | | TRBV10-3*02 | U17047 |
| | | TRBV10-3*03 | L33101 |
| | | TRBV10-3*04 | L33102 |
| TRBV11 | TRBV11-1 | TRBV11-1*01 | M33233 |
| | TRBV11-2 | TRBV11-2*01 | L36092 |
| | | TRBV11-2*02 | M33235 |
| | | TRBV11-2*03 | IMGT000021 |
| | TRBV11-3 | TRBV11-3*01 | U03115 |
| | | TRBV11-3*02 | X58797 |
| | | TRBV11-3*03 | M62377 |
| | | TRBV11-3*04 | AB305924 |
| TRBV12 | TRBV12-1 | TRBV12-1*01 | X07224 |
| | TRBV12-2 | TRBV12-2*01 | X06936 |
| | TRBV12-3 | TRBV12-3*01 | X07192 |
| | TRBV12-4 | TRBV12-4*01 | K02546 |
| | | TRBV12-4*02 | M14264 |
| | TRBV12-5 | TRBV12-5*01 | X07223 |
| TRBV13 | TRBV13 | TRBV13*01 | U03115 |
| | | TRBV13*02 | M62378 |
| TRBV14 | TRBV14 | TRBV14*01 | X06154 |
| | | TRBV14*02 | X57722 |
| TRBV15 | TRBV15 | TRBV15*01 | U03115 |
| | | TRBV15*02 | IMGT000021 |
| | | TRBV15*03 | M62376 |
| TRBV16 | TRBV16 | TRBV16*01 | L26231 |
| | | TRBV16*02 | U03115 |
| | | TRBV16*03 | L26054 |
| TRBV17 | TRBV17 | TRBV17*01 | U03115 |
| | | TRBV17*02 | IMGT000021 |
| TRBV18 | TRBV18 | TRBV18*01 | L36092 |
| TRBV19 | TRBV19 | TRBV19*01 | L36092 |
| | | TRBV19*02 | U48259 |
| | | TRBV19*03 | M97725 |
| TRBV20 | TRBV20-1 | TRBV20-1*01 | M11955 |
| | | TRBV20-1*02 | X72719 |
| | | TRBV20-1*03 | M11954 |
| | | TRBV20-1*04 | M14263 |
| | | TRBV20-1*05 | X57604 |
| | | TRBV20-1*06 | D13088 |
| | | TRBV20-1*07 | X74852 |
| TRBV21 | TRBV21-1 | TRBV21-1*01 | L36092 |
| | | TRBV21-1*02 | IMGT000021 |
| TRBV22 | TRBV22-1 | TRBV22-1*01 | L36092 |
| TRBV23 | TRBV23-1 | TRBV23-1*01 | L36092 |
| TRBV24 | TRBV24-1 | TRBV24-1*01 | M11951 |
| | | TRBV24-1*02 | IMGT000021 |
| TRBV25 | TRBV25-1 | TRBV25-1*01 | L36092 |
| TRBV26 | TRBV26 | TRBV26*01 | L36092 |
| TRBV27 | TRBV27 | TRBV27*01 | L36092 |
| TRBV28 | TRBV28 | TRBV28*01 | U08314 |
| TRBV29 | TRBV29-1 | TRBV29-1*01 | L36092 |
| | | TRBV29-1*02 | M13847 |
| | | TRBV29-1*03 | X04926 |
| TRBV30 | TRBV30 | TRBV30*01 | L36092 |
| | | TRBV30*02 | Z13967 |
| | | TRBV30*03 | IMGT000027 |
| | | TRBV30*04 | M13554 |
| | | TRBV30*05 | L06893 |

The term “accession number” or “accession code” and all grammatical equivalents, means an identifiable or searchable number or code, referring to a peptide or nucleotide sequence. Methods of searching the accession number are can be achieved via numerous methods, including but not limited to IMGT (www, Imgt.org), GenBank (www.ncbi.nlm.nih.gov/genbank), and The Kabat Database (G. Johnson and T. T. Wu, 2002; http://www.kabatdatabase.com).

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity.

An antibody can be of any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules such as toxins, radioisotopes, etc.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain an antigen recognition site of an intact antibody (e.g., complementarity determining regions (CDRs) sufficient to bind antigen). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

The term “intrabody” refers to an antibody or antibody binding fragment” that works within the cell to bind to an intracellular protein.

“Antigen” (Ag) as used herein refers to a molecule that can provoke an immune response, e.g., involving activation of certain immune cells and/or antibody generation. Any natural or synthetic immunogenic substance, including but not limited to a hapten, or any macromolecule, including but not limited to almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” An antigen does not need to be encoded solely by a full-length nucleotide sequence of a gene, nor does an antigen need to be encoded by a gene at all. An antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

As used herein, an “immune cell” refers to any of various cells that function in the immune system, e.g., to protect against agents of infection and foreign matter. This term includes leukocytes, e.g., neutrophils, eosinophils, basophils, lymphocytes, and monocytes. Innate leukocytes include phagocytes (e.g., macrophages, neutrophils, and dendritic cells), mast cells, eosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

As used herein, the term “specifically binds” refers to an antigen binding molecule that recognizes and binds a protein of a binding partner (such as a variable β region) present in a sample, but the antigen binding molecule does not substantially recognize or bind to other molecules in the sample.

The term “high affinity” as used herein refers to high specificity in binding or interacting or attraction of one molecule to a target molecule.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that CDRs of the light and heavy chains are primarily responsible for the interaction and specificity of the antibody with antigen.

The terms “VH” and “VH domain” are used interchangeably to refer to the heavy chain variable region of an antibody or antigen-binding fragment thereof.

The terms “VL” and “VL domain” are used interchangeably to refer to the light chain variable region of an antibody or antigen-binding fragment thereof.

The “hypervariable regions” in each chain are held together in close proximity by FRs, and with the hypervariable regions from the other chain, contribute to the formation of the antigen-binding site of antibodies (see Kabat et al, Sequences of Proteins of Immunological Interest, 1992). The term “hypervariable region” as used herein refers to the amino acid residues of an antibody, which are responsible for antigen binding. The hypervariable region generally comprises amino acid residues from a “complementary determining region” or “CDR”, the latter being of highest sequence variability and/or involved in antigen recognition. A number of CDR definitions are in use and are encompassed herein. The Kabat definition is based on sequence variability and is the most commonly used (Kabat E A et al., supra). Chothia refers instead to the location of the structural loops (Chothia C & LeskAM (1987) J. Mol. Biol. 196: 901-917). The AbM definition is a compromise between the Kabat and the Chothia definitions and is used by Oxford Molecular's AbM antibody modelling software (Martin A C R et al., (1989) Proc. Natl Acad. Sci. USA, 86: 9268-72; Martin A C R et al., (1991) Methods Enzymol. 203: 121-153; Pedersen J T et al., (1992) Immunomethods, 1: 126-136; Rees A R et al., (1996) In Sternberg M. J. E. (ed.), Protein Structure Prediction. Oxford University Press, Oxford, 141-172). The contact definition has been recently introduced (Maccallum R M et al., (1996) J. Mol. Biol. 262: 732-7 45) and is based on an analysis of the complex structures available in the Protein Databank. The definition of the CDR by IMGT®, the international ImMunoGeneTics information System® (http://www, Imgt.org) is based on the IMGT numbering for all immunoglobulin and T cell receptor V-REGIONs of all species (IMGT®, the international ImMunoGeneTics information System®; Lefranc M P et al., (1991) Nucleic Acids Res. 27(1): 209-12; Ruiz M et al., (2000) Nucleic Acids Res. 28(1): 219-21; Lefranc M P (2001) Nucleic Acids Res. 29(1): 207-9; Lefranc M P (2003) Nucleic Acids Res. 31(1): 307-10; Lefranc M P et al., (2005) Dev. Comp. Immunol. 29(3): 185-203; Kaas Q et al., (2007) Briefings in Functional Genomics & Proteomics, 6(4): 253-64).

The Complementarity Determining Regions (CDRs) disclosed herein can be defined according to IMGT®. The CDRs can be defined according to Chothia. The CDRs can be defined according to Kabat. For example, for the light chains, the variable domain residues for each of the CDRs can be (numbering according to Kabat E A, et al., supra): LCDR1: 27-32, LCDR2: 50-52, LCDR3: 89-97. The “non-CDR region” of the VL region as used herein comprise the amino acid sequences: 1-26 (FRI), 33-49 (FR2), 53-88 (FR3), and 98-approximately 107 (FR4). For the heavy chains, the variable domain residues for each of the three CDRs can be HCDR1: 26-35, HCDR2: 51-57 and HCDR3: 93-102.

Different software can be used to generate alternate CDR sequences for the framework sequences of a variable region with different CDR sequences resulting from the use of the different software programs. The use of alternate CDR sequences can improve binding affinities of an antibody to at least one antigen. Alternate CDR sequences are used for affinity optimization of one or both antigen binding sites of an antibody according to the present disclosure. The alternate CDRs are defined according to Kabat, Chothia, Paratome, AbM, Contact and/or IMGT annotations. The CDRs are defined according to more than one annotation.

As used herein, the term “Fab region” refers to VH and CHI domains of a heavy chain (“Fab heavy chain”), or VL and CL domains of a light chain (“Fab light chain”) of an immunoglobulin.

As used herein, the term “scFv” or “single chain antibody fragment” refers to a single chain consisting of a heavy chain variable region and a light chain variable region of an antibody being linearly linked together by a linker (e.g., a short peptide of 10-25 amino acids), which exhibits specific binding to an antigen. The scFv can be refer to a single chain comprising a signal peptide, a heavy chain variable region and a light chain variable region of an antibody being linearly linked together by a linker.

As used herein, the terms “constant region” and “constant domain” are interchangeable and have their meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain.

As used herein, the term “heavy chain” when used in reference to an antibody can refer to any distinct type, e.g. alpha (a), delta (d), epsilon (e), gamma (g), and mu (m), based on the amino acid sequence of the constant domain, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. The heavy chain can be a human heavy chain.

As used herein, the term “light chain” when used in reference to an antibody can refer to any distinct type, e.g. kappa (K) or lambda (l) based on the amino acid sequence of the constant domains. Light chain amino acid sequences are well known in the art. The light chain can be a human light chain.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the non-human CDR residues to refine and optimize the specificity, affinity, and/or capability of the antibody or antigen-binding fragment thereof. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). A “humanized antibody” can be a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

The term “ADCC” or “antibody dependent cell-mediated cytotoxicity” as used herein is the cell-mediated reaction wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

The term “ADCP” or antibody dependent cell-mediated phagocytosis as used herein is meant the cell-mediated reaction wherein nonspecific cytotoxic cells that express FcγRs recognize bound antibody on a target cell and subsequently cause phagocytosis of the target cell.

In some aspects the humanized antibody heavy chain Fc region with ADCC enhancing mutations comprises, but is not limited to, one or more, of the following mutations H268F, S324T, S239D, and 1332E and any combination thereof.

In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII FcR expression on hematopoietic cells is summarized in Table 3 on page 464 of Ravetch, J. V. and Kinet, J. P., Annu. Rev. Immunol. 9 (1991) 457-492. Non-limiting examples of in vitro assays to assess ADCC activity of a molecule of interest is described in U.S. Pat. No. 5,500,362 (see, e.g. Hellstrom, I. et al., Proc. Natl. Acad. Sci. USA 83 (1986) 7059-7063; and Hellstrom, I. et al., Proc. Natl. Acad. Sci. USA 82 (1985) 1499-1502); U.S. Pat. No. 5,821,337 (see Bruggemann, M. et al., J. Exp. Med. 166 (1987) 1351-1361). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells.

Alternatively, or additionally, ADCC activity of the molecule of interest may be assessed in vivo, e.g., in an animal model such as that disclosed in Clynes, R. et al., Proc. Natl. Acad. Sci. USA 95 (1998) 652-656. C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro, H. et al., J. Immunol. Methods 202 (1996) 163-171; Cragg, M. S. et al., Blood 101 (2003) 1045-1052; and Cragg, M. S. and M. J. Glennie, Blood 103 (2004) 2738-2743). FcRn binding and in vivo clearance/half-life determinations can also be performed using methods known in the art (see, e.g., Petkova, S. B. et al., Int. Immunol. 18 (2006) 1759-1769).

Antibodies with reduced effector function include those with substitution of one or more of Fc-region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

By “effector function” as used herein is meant a biochemical event that results from the interaction of an antibody Fc region with an Fc receptor or ligand. Effector functions include but are not limited to ADCC, ADCP, and CDC.

Certain antibody variants with improved or diminished binding to FcRs are described in, e.g., U.S. Pat. No. 6,737,056; WO 2004/056312, and Shields, R. L. et al., J. Biol. Chem. 276 (2001) 6591-6604.

In certain aspects, an antibody variant comprises an Fc-region with one or more amino acid substitutions which improve ADCC, e.g., substitutions at positions 298, 333, and/or 334 of the Fc-region (EU numbering of residues).

“Binding affinity” generally refers to the strength of the sum total of non-covalent interactions between a single binding site of a molecule (e.g., an antibody or antigen binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen binding fragment thereof and antigen). The affinity of a molecule X for its partner Y can generally be represented by the dissociation constant (KD). Affinity can be measured and/or expressed in a number of ways known in the art, including, but not limited to, equilibrium dissociation constant (KD), and equilibrium association constant (KA). The KD is calculated from the quotient of k_{off}/kₒₙ, whereas KA is calculated from the quotient of kₒₙ/k_{off}. kₒₙ refers to the association rate constant of, e.g., an antibody or antigen binding fragment thereof to an antigen, and k_{off} refers to the dissociation of, e.g., an antibody or antigen-binding fragment thereof from an antigen. The kₒₙ and k_{off} can be determined by techniques known to one of ordinary skill in the art, such as BIAcore® or KinExA.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope).

The epitope to which an antibody or antigen-binding fragment thereof binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping).

As used herein, an antibody is to “competitively inhibit” binding of a reference antibody to a given epitope if it preferentially binds to that epitope or an overlapping epitope to the extent that it blocks, to some degree, binding of the reference antibody to the epitope. Competitive inhibition can be determined by any method known in the art, for example, competition ELISA assays. An antibody can be said to competitively inhibit binding of the reference antibody to a given epitope by at least 90%, at least 80%, at least 70%, at least 60%, or at least 50%.

As used herein, the term “promoter/regulatory sequence” refers to a nucleic acid sequence required to express a gene product operably linked to a promoter/regulatory sequence. The term “constitutive” promoter refers to a nucleotide sequence that, when operably linked to a polynucleotide encoding or specifying a gene product, results in the production of a gene product in the cell under most or all physiological conditions of the cell. The term “inducible” promoter means that when operably linked to a polynucleotide encoding a specified gene product, it basically results in the production of a gene in the cell only when the inducer corresponding to the promoter is present in the cell The nucleotide sequence of the product.

As used herein, the term “expression” refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). Expression of a gene produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

The nucleic acid molecule can be cloned into any number of different types of vectors. For example, the nucleic acid molecule can be cloned into a vector including, but not limited to a plasmid, a phagemid, a phage derivative, an animal virus, and a cosmid. Vectors of particular interest include expression vectors, replication vectors, probe generation vectors, and sequencing vectors.

The term “vector” as used herein, includes, but is not limited to, a viral vector, a plasmid, an RNA vector or a linear or circular DNA or RNA molecule which can include chromosomal, non-chromosomal, semi-synthetic or synthetic nucleic acids. In some cases, the vectors are those capable of autonomous replication (episomal vector) and/or expression of nucleic acids to which they are linked (expression vectors). Large numbers of suitable vectors are known to those of skill in the art and are commercially available. Viral vectors include retrovirus, adenovirus, parvovirus (e.g., adenoassociated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, and lentivirus.

As used herein, the term “expression vector” refers to a vector comprising a recombinant polynucleotide comprising an expression control sequence operably linked to the nucleotide sequence to be expressed. The expression vector contains sufficient cis-acting elements for expression; other elements for expression can be provided by the host cell or in an in vitro expression system. Expression vectors include expression vectors known in the art, including cosmids, plasmids (for example, naked or contained in liposomes), and viruses incorporating recombinant polynucleotides (for example, lentivirus, retrovirus, adenovirus, and adeno-associated virus).

The expression vector may be provided to a cell in the form of a viral vector. Viral vector technology is well known in the art and is described, for example, in Sambrook et ak, 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, individual elements can function either cooperatively or independently to activate transcription.

As used herein, the term “transfer vector” refers to a composition containing an isolated nucleic acid and a substance that can be used to deliver the isolated nucleic acid to the inside of a cell. Many vectors are known in the art, including but not limited to linear polynucleotides, polynucleotides associated with ionic or amphiphilic compounds, plasmids, and viruses. Therefore, a transfer vector can include autonomously replicating plasmids or viruses. The term transfer vector should also be interpreted to further include non-plasmid and non-viral compounds that facilitate the transfer of nucleic acids into cells, such as polylysine compounds, liposomes, and the like. Examples of virus transfer vectors include, but are not limited to, adenoviral vectors, adeno-associated virus vectors, retroviral vectors, lentiviral vectors, and the like.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

As used herein, the term “operably linked” or “transcription control” refers to a functional linkage between a regulatory sequence and a heterologous nucleic acid sequence, which results in the expression of the latter. For example, when the first nucleic acid sequence and the second nucleic acid sequence are arranged in a functional relationship, the first nucleic acid sequence and the second nucleic acid sequence are operably linked. For example, if a promoter affects the transcription or expression of a coding sequence, the promoter is operably linked to the coding sequence. The operably linked DNA sequences may be adjacent to each other, and for example, in the case where two protein coding regions need to be linked, the DNA sequences are in the same reading frame.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

As used herein, the term “host cell” can be any type of cell, e.g., a primary cell, a cell in culture, or a cell from a cell line. The term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY).

Biological methods for introducing a polynucleotide of interest into a host cell include the use of DNA and RNA vectors. RNA vectors include vectors having a RNA promoter and/other relevant domains for production of a RNA transcript. Viral vectors, and especially retroviral vectors, have become the most widely used method for inserting genes into mammalian, e.g., human cells. Other viral vectors may be derived from lentivirus, poxviruses, herpes simplex virus, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances, which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

The terms “AAV,” “AAV construct,” or “recombinant AAV” or “AAV” refer to adeno-associated virus of any of the known serotypes, including AAV-1, AAV-2, AAV-3, AAV-4, AAV-5, AAV-6, AAV-7, AAV-8, AAV-9, AAV-10, AAV-11, or AAV-12, scAAV, rh10, chimeric or hybrid AAV, or any combination, derivative, or variant thereof. AAV is a small non-enveloped single-stranded DNA virus. They are non-pathogenic parvoviruses and can require helper viruses, such as adenovirus, herpes simplex virus, vaccinia virus, and CMV, for replication. Wild-type AAV is common in the general population, and is not associated with any known pathologies. A hybrid AAV is an AAV comprising a capsid protein of one AAV serotype and genomic material from another AAV serotype. A chimeric AAV comprises genetic and/or protein sequences derived from two or more AAV serotypes, and can include mutations made to the genetic sequences of those two or more AAV serotypes. An exemplary chimeric AAV can comprise a chimeric AAV capsid, for example, a capsid protein with one or more regions of amino acids derived from two or more AAV serotypes. An AAV variant is an AAV comprising one or more amino acid mutations in its genome or proteins as compared to its parental AAV, e.g., one or more amino acid mutations in its capsid protein as compared to its parental AAV. AAV, as used herein, includes avian AAV, bovine AAV, canine AAV, equine AAV, primate AAV, non-primate AAV, and ovine AAV, wherein primate AAV refers to AAV that infect non-primates, and wherein non-primate AAV refers to AAV that infect non-primate animals, such as avian AAV that infects avian animals. In some cases, the wild-type AAV contains rep and cap genes, wherein the rep gene is required for viral replication and the cap gene is required for the synthesis of capsid proteins. As used herein, the terms “recombinant AAV” and “rAAV” are interchangeable.

The terms “recombinant AAV vector” or “AAV vector” or “AAV vector” refer to a vector derived from any of the AAV serotypes mentioned above. In some cases, an AAV vector can comprise one or more of the AAV wild-type genes deleted in whole or part, such as the rep and/or cap genes, but contains functional elements that are required for packaging and use of AAV virus for gene therapy. For example, functional inverted terminal repeats or ITR sequences that flank an open reading frame or exogenous sequences cloned in are known to be important for replication and packaging of an AAV virion, but the ITR sequences can be modified from the wild-type nucleotide sequences, including insertions, deletions, or substitutions of nucleotides, so that the AAV is suitable for use for the disclosure described herein, such as a gene therapy or gene delivery system. A gene therapy could include exemplary AAV DNA encoded sequences for in vivo delivery via intradermal, intramuscular, intravenous, intranodal (lymph node), intratumoral or other injection into patients such that therapeutic CAR or antibody formation may occur in vivo using the patient's own cells.

In some aspects, a self-complementary vector (sc) can be used, such as a self-complementary AAV vector, which can bypass the requirement for viral second-strand DNA synthesis and can lead to higher rate of expression of a transgene protein, as described in Wu, Hum Gene Ther. 2007, 18(2):171-82, incorporated by reference herein. In some aspects, AAV vectors can be generated to allow selection of an optimal serotype, promoter, and transgene. In some cases, the vector can be targeted vector or a modified vector that selectively binds or infects immune cells.

The terms “AAV virion” or “AAV virion” refer to a virus particle comprising a capsid comprising at least one AAV capsid protein that encapsidates an AAV vector as described herein, wherein the vector can further comprise a heterologous polynucleotide sequence or a transgene in some embodiments.

Modified adeno-associated viral vectors can be useful to treat various indications, including for instance cancer or T cell mediated autoimmune diseases in subjects. For example, autologous peripheral blood lymphocytes (PBL) can be modified using adeno-associated viral methods to express exogenous cellular receptors that recognize unique epitopes, mutations, neo-antigens, on cancer cells and can be used in the disclosed compositions and methods of an intracellular genomic transplant. These compositions utilizing mutated and chimeric adeno-associated viral vectors and methods utilizing said AAV vectors for intracellular genomic transplant can provide a therapy with many advantages. For example, they can provide high efficiency gene transfer, expression, increased cell survival rates, an efficient introduction of recombinogenic double strand breaks, and a process that favors the Homology Directed Repair (HDR) over Non-Homologous End Joining (NHEJ) mechanism, and efficient recovery and expansion of homologous recombinants. In some aspects of both lentivirual vector and AAV vectors, P2A and GFP can be removed or replaced by P2A-human CD47 to reduce or diminish host-versus-graft (HVG) effects, and AmpR can be replaced by the Kanamycin resistant gene. In some aspects of both lentivirual vector and AAV vectors, P2A can be removed or replaced by P2A-human CD47 to reduce or diminish host-versus-graft (HVG) effects, and AmpR can be replaced by the Kanamycin resistant gene. In some aspects of both lentivirual vector and AAV vectors, GFP can be removed or replaced by P2A-human CD47 to reduce or diminish host-versus-graft (HVG) effects, and AmpR can be replaced by the Kanamycin resistant gene.

As used herein, the term “autologous” refers to any material derived from an individual that will later be reintroduced into that same individual.

Intracellular genomic transplant can be a method of genetically modifying cells and nucleic acids for therapeutic applications. The compositions and methods described throughout can use mutated and chimeric adeno-associated viral vectors to improve upon existing viral delivery mechanisms. Effective adoptive cell transfer-based immunotherapies (ACT) can be useful to treat various indications in a subject. For example, autologous peripheral blood lymphocytes (PBL) can be modified using modified adeno-associated viral vectors with mutations in at least a portion of a capsid protein or adeno-associated viral vectors with capsid proteins from at least two different serologies.

At the cellular level, AAV can undergo 5 steps prior to achieving gene expression: 1) binding or attachment to cellular surface receptors, 2) endocytosis, 3) trafficking to the nucleus, 4) uncoating of the virus to release the genome and 5) conversion of the genome from single-stranded to double-stranded DNA as a template for transcription in the nucleus. The cumulative efficiency with which AAV can successfully execute each individual step can determine the overall transduction efficiency. Rate limiting steps in AAV transduction can include the absence or low abundance of required cellular surface receptors for viral attachment and internalization, inefficient endosomal escape leading to lysosomal degradation, and slow conversion of single-stranded to double-stranded DNA template. Therefore, vectors with modifications to the genome and/or the capsids can be designed to facilitate more efficient or more specific transduction or cells or tissues for gene therapy.

As used herein, the terms, “transfection” and “transduction,” refer to the processes by which an exogenous nucleic acid sequence is introduced into a host cell. The nucleic acid can be integrated into the host cell DNA or can be maintained extrachromosomally. The nucleic acid can be maintained transiently or can be a stable introduction. Transfection can be accomplished by a variety of means known in the art including but not limited to calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Transduction refers to the delivery of a gene(s) using a viral or retroviral vector by means of viral infection rather than by transfection. In some aspects, retroviral vectors are transduced by packaging the vectors into virions prior to contact with a cell. For example, a nucleic acid encoding a TCR Vβ CAR carried by a retroviral vector can be transduced into a cell through infection and pro virus integration.

The term “chimeric antigen receptor” (CAR) refers to a chimeric molecule that includes a binding domain which binds to a component (e.g., a ligand) present on a target cell (for example, the binding domain can include an antibody or antigen-binding fragment thereof specific for a desired antigen (e.g., Vβ region of a TCR)) and a T cell receptor-activating intracellular domain. The CAR exhibits a specific anti-target cellular immune activity. CARs can include an extracellular ligand-binding domain (e.g., a single chain antibody-binding domain (scFv)) fused to the intracellular signaling domain of the T cell antigen receptor complex zeta chain. The CAR can include an extracellular ligand-binding domain, a hinge, a transmembrane domain, and a cytoplasmic domain. The cytoplasmic domain can comprise a costimulatory domain (e.g., 4-1BB) and a signaling domain (e.g., CD3zeta). The CAR, when expressed in T cells, can redirect antigen recognition based on the antibody's specificity.

The expression of natural or synthetic nucleic acid molecule encoding CARs or antibody or antigen binding fragments is typically achieved by operably linking a nucleic acid encoding the CAR polypeptide or the antibody or antigen biding fragment polypeptide(s) or portions thereof to a promoter (e.g. EF1 alpha promoter), and incorporating the construct into an expression vector. The vector is one generally capable of replication in a mammalian cell, and/or also capable of integration into the cellular genome of the mammal. Typical vectors contain transcription and translation terminators, initiation sequences, and promoters useful for regulation of the expression of the desired nucleic acid sequence.

A “costimulatory domain” or “costimulatory molecule” refers to the cognate binding partner on a T-cell that specifically binds with a costimulatory ligand, thereby mediating a costimulatory response by the cell (e.g., a proliferation response). Costimulatory molecules include, but are not limited to, an MHC class I molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like. The costimulatory molecule can be a cell surface molecule, other than an antigen receptor or their ligands, that is promotes an efficient immune response.

A “costimulatory ligand” refers to a molecule on an antigen presenting cell that specifically binds a cognate costimulatory molecule on a T-cell.

A costimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3.

The term “extracellular ligand-binding domain,” or “extracellular domain” as used herein, refers to an oligo- or polypeptide that is capable of binding a ligand, e.g., a cell surface molecule. For example, the extracellular ligand-binding domain can be chosen to recognize a ligand that acts as a cell surface marker on target cells associated with a particular disease state (e.g., cancer or T-cell mediated autoimmune diseases). Examples of cell surface markers that can act as ligands include those associated with viral, bacterial and parasitic infections, autoimmune disease and cancer cells.

A “signal transducing domain” or “signaling domain”, as used herein with respect to CARs, is responsible for intracellular signaling following the binding of an extracellular ligand binding domain to a target. The signaling domain as used in a CAR results in the activation of the immune cell and immune response. In other words, the signal transducing domain can responsible for the activation of at least one of the normal effector functions of the immune cell in which the CAR is expressed. For example, the effector function of a T cell can be a cytolytic activity or helper activity including the secretion of cytokines. Thus, the signal transducing domain can refer to the portion of a protein which transduces the effector function signal and directs the cell to perform a specialized function. Examples of signal transducing domains for use in a CAR can be the cytoplasmic sequences of the T cell receptor and co-receptors that act in concert to initiate signal transduction following antigen receptor engagement, as well as any derivative or variant of these sequences and any synthetic sequence that has the same functional capability. In some cases, signaling domains comprise two distinct classes of cytoplasmic signaling sequences, those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs (ITAMs).

“ITAMs” refer to signaling motifs found in the intracytoplasmic tail of a variety of receptors that serve as binding sites for syk/zap70 class tyrosine kinases. Exemplary ITAMs include those derived from TCRzeta, FcRgamma, FcRbeta, FcRepsilon, CD3gamma, CD3delta, CD3epsilon, CD3zeta, CD5, CD22, CD79a, CD79b and CD66d. The signaling domain of the CAR can comprise the CD3zeta signaling domain.

The “transmembrane region” or “transmembrane domain” as used herein is a portion of a CAR that anchors the extracellular binding portion to the plasma membrane of an immune effector cell, and facilitates binding of the binding domain to the target antigen. In some aspects, the transmembrane domain can be a CD3zeta transmembrane domain, however other transmembrane domains that can be employed include those obtained from CD8α, CD4, CD28, CD45, CD9, CD16, CD22, CD33, CD64, CD80, CD86, CD134, CD137, or CD154.

The binding domain of a CAR can be followed by a “spacer,” or, “hinge,” which refers to the region that separates or moves the antigen binding domain away from the effector cell surface. In some aspects, the hinge enables cell/cell contact, antigen binding and activation (Patel et al., Gene Therapy, 1999; 6: 412-419). The hinge region in a CAR can be between the transmembrane (TM) and the binding domain. In some aspects, a hinge region is an immunoglobulin hinge region and can be a wild type immunoglobulin hinge region or an altered wild type immunoglobulin hinge region. Other exemplary hinge regions used in the CARs disclosed herein can include the hinge region derived from the extracellular regions of type 1 membrane proteins such as CD8α, CD4, CD28 and CD7, which may be wild-type hinge regions from these molecules or may be altered. In some aspects, the hinge region comprises a CD8α hinge.

It is to be understood that headers are provided solely for ease of reading, and are not intended to be limiting. Aspects disclosed under one or more headers can be applicable to or combinable with aspects disclosed under one or more other headers.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### I. Chimeric Antigen Receptor T-Cells (CAR)

Chimeric antigen receptors (CARs) redirect T cell specificity toward antibody-recognized antigens expressed on the surface of cells (e.g., variable β regions of TCR's).

In some aspects described herein, the present disclosure includes a chimeric antigen receptor (CAR) which is specific for a Vβ region of a TCR. In some aspects described herein, the CAR is, for example, an extracellular domain, a transmembrane domain, an intracellular signaling domain (a signaling domain derived from CD3zeta or FcRgamma), and/or one or more co-stimulatory signaling domains derived from a co-stimulatory molecule including, but not limited to, 4-1BB. In some aspects, the CAR includes a hinge or spacer region between the extracellular binding domain and the transmembrane domain, such as a CD8α hinge.

In some aspects described herein, the costimulatory domain comprises a 4-1BB costimulatory domain. In some aspects described herein, the 4-1BB costimulatory domain, comprises an amino acid sequence according to SEQ ID NO: 8.

In some aspects described herein, the transmembrane domain comprises an hCD28 transmembrane. In some aspects described herein, the transmembrane domain comprises an hCD28 amino acid sequence according to SEQ ID NO: 3.

In some aspects described herein, the signaling domain comprises a CD247 signaling domain. In some aspects described herein, the signaling domain comprises a CD247 signaling domain amino acid sequence according to SEQ ID NO: 9.

In some aspects described herein, scFv comprises a CD28 signal peptide. In some aspects described herein, the scFv comprises a CD28 signal peptide an amino acid sequence according to SEQ ID NO:7.

In some aspects, the binding domain or the extracellular domain of the CAR, for example, provides the CAR with the ability to bind to the target antigen of interest. A binding domain (e.g., a ligand-binding domain or antigen-binding domain), for example, can be any protein, polypeptide, oligopeptide, or peptide that possesses the ability to specifically recognize and bind to a biological molecule (e.g., a cell surface receptor or tumor protein, or a component thereof, or a variable beta chain of a t-cell receptor). A binding domain can include any naturally occurring, synthetic, semi-synthetic, or recombinantly produced binding partner for a biological molecule of interest. For example, and as further disclosed herein, a binding domain can be antibody light chain and heavy chain variable regions, or the light and heavy chain variable regions can be joined together in a single chain and in either orientation (e.g., VL-VH or VH-VL).

In some aspects described herein, a chimeric antigen receptor (CAR), comprises, (i) an antigen binding molecule that specifically binds to a TCR Vβ region, (ii) an extracellular domain, (iii) a transmembrane domain, (iv) a costimulatory domain and (v) an activating domain, the antigen binding molecule is a single chain variable fragment (scFv) comprising: a variable heavy chain (VH) according to any one of SEQ ID NOs: 45-131 and 534-544, or a variable light chain (VL) according to any one of SEQ ID NOs: 132-227 and 545-558.

In some aspects described herein, the CAR comprises, (i) the variable heavy chain (VH) comprising CDR1, CDR2 and CDR3 according to SEQ ID NO:10, SEQ ID NO:11, and SEQ ID NO:12, respectively, and/or (ii) the variable light chain (VL) comprises CDR1, CDR2 and CDR3 according to SEQ ID NO:13, SEQ ID NO:14, SEQ ID NO:15, respectively; or (i) the variable heavy chain (VH) comprises CDR1, CDR2 and CDR3 according to SEQ ID NO:370, SEQ ID NO:371, and SEQ ID NO:372, respectively, and/or (ii) the variable light chain (VL) comprises CDR1, CDR2 and CDR3 according to SEQ ID NO:418, SEQ ID NO:419, SEQ ID NO:420, respectively; or (i) the variable heavy chain (VH) comprises CDR1, CDR2 and CDR3 according to SEQ ID NO:373, SEQ ID NO:374, and SEQ ID NO:375 respectively, and/or (ii) the variable light chain (VL) comprises CDR1, CDR2 and CDR3 according to SEQ ID NO:421, SEQ ID NO:422, SEQ ID NO:423, respectively; or (i) the variable heavy chain (VH) comprises CDR1, CDR2 and CDR3 according to SEQ ID NO:376, SEQ ID NO:377, and SEQ ID NO:378, respectively, and/or (ii) the variable light chain (VL) comprises CDR1, CDR2 and CDR3 according to SEQ ID NO:424, SEQ ID NO:425, SEQ ID NO:426, respectively; or the variable heavy chain (VH) comprises CDR1, CDR2 and CDR3 according to SEQ ID NO:379, SEQ ID NO:380, and SEQ ID NO:381 respectively, and/or (ii) the variable light chain (VL) comprises CDR1, CDR2 and CDR3 according to SEQ ID NO:427, SEQ ID NO:428, SEQ ID NO:429, respectively; or (i) the variable heavy chain (VH) comprises CDR1, CDR2 and CDR3 according to SEQ ID NO:382, SEQ ID NO:383, and SEQ ID NO:384, respectively, and/or (ii) the variable light chain (VL) comprises CDR1, CDR2 and CDR3 according to SEQ ID NO:430, SEQ ID NO:431, SEQ ID NO:432, respectively; or (i) the variable heavy chain (VH) comprises CDR1, CDR2 and CDR3 according to SEQ ID NO:385, SEQ ID NO:386, and SEQ ID NO:387, respectively, and/or (ii) the variable light chain (VL) comprises CDR1, CDR2 and CDR3 according to SEQ ID NO:433, SEQ ID NO:434, SEQ ID NO:435, respectively; or (i) the variable heavy chain (VH) comprises CDR1, CDR2 and CDR3 according to SEQ ID NO:388, SEQ ID NO:389, and SEQ ID NO:390, respectively, and/or (ii) the variable light chain (VL) comprises CDR1, CDR2 and CDR3 according to SEQ ID NO:436, SEQ ID NO:437, SEQ ID NO:438, respectively; or (i) the variable heavy chain (VH) comprises CDR1, CDR2 and CDR3 according to SEQ ID NO:391, SEQ ID NO:392, and SEQ ID NO:393 respectively, and/or (ii) the variable light chain (VL) comprises CDR1, CDR2 and CDR3 according to SEQ ID NO:439, SEQ ID NO:440, SEQ ID NO:441, respectively; or (i) the variable heavy chain (VH) comprises CDR1, CDR2 and CDR3 according to SEQ ID NO:394, SEQ ID NO:395, and SEQ ID NO:396, respectively, and/or (ii) the variable light chain (VL) comprises CDR1, CDR2 and CDR3 according to SEQ ID NO:442, SEQ ID NO:443, SEQ ID NO:444, respectively; or (i) the variable heavy chain (VH) comprises CDR1, CDR2 and CDR3 according to SEQ ID NO:397, SEQ ID NO:398, and SEQ ID NO:399, respectively, and/or (ii) the variable light chain (VL) comprises CDR1, CDR2 and CDR3 according to SEQ ID NO:445, SEQ ID NO:446, SEQ ID NO:447, respectively; or (i) the variable heavy chain (VH) comprises CDR1, CDR2 and CDR3 according to SEQ ID NO:400, SEQ ID NO:401, and SEQ ID NO:402, respectively, and/or (ii) the variable light chain (VL) comprises CDR1, CDR2 and CDR3 according to SEQ ID NO:448, SEQ ID NO:449, SEQ ID NO:450, respectively; or (i) the variable heavy chain (VH) comprises CDR1, CDR2 and CDR3 according to SEQ ID NO:403, SEQ ID NO:404, and SEQ ID NO:405 respectively, and/or (ii) the variable light chain (VL) comprises CDR1, CDR2 and CDR3 according to SEQ ID NO:451, SEQ ID NO:452, SEQ ID NO:453, respectively; or (i) the variable heavy chain (VH) comprises CDR1, CDR2 and CDR3 according to SEQ ID NO:406, SEQ ID NO:407, and SEQ ID NO:408, respectively, and/or (ii) the variable light chain (VL) comprises CDR1, CDR2 and CDR3 according to SEQ ID NO:454, SEQ ID NO:455, SEQ ID NO:456, respectively; or (i) the variable heavy chain (VH) comprises CDR1, CDR2 and CDR3 according to SEQ ID NO:409, SEQ ID NO:410, and SEQ ID NO:411, respectively, and/or (ii) the variable light chain (VL) comprises CDR1, CDR2 and CDR3 according to SEQ ID NO:457, SEQ ID NO:458, SEQ ID NO:459, respectively; or (i) the variable heavy chain (VH) comprises CDR1, CDR2 and CDR3 according to SEQ ID NO:412, SEQ ID NO:413, and SEQ ID NO:414, respectively, and/or (ii) the variable light chain (VL) comprises CDR1, CDR2 and CDR3 according to SEQ ID NO:460, SEQ ID NO:461, SEQ ID NO:462, respectively; or (i) the variable heavy chain (VH) comprises CDR1, CDR2 and CDR3 according to SEQ ID NO:415, SEQ ID NO:416, and SEQ ID NO:417, respectively, and/or (ii) the variable light chain (VL) comprises CDR1, CDR2 and CDR3 according to SEQ ID NO:463, SEQ ID NO:464, SEQ ID NO:465, respectively; or (i) the variable heavy chain (VH) comprises CDR1, CDR2 and CDR3 according to SEQ ID NO:584, SEQ ID NO:585, and SEQ ID NO:586, respectively, and/or (ii) the variable light chain (VL) comprises CDR1, CDR2 and CDR3 according to SEQ ID NO:596, SEQ ID NO:597, SEQ ID NO:598, respectively; or (i) the variable heavy chain (VH) comprises CDR1, CDR2 and CDR3 according to SEQ ID NO:587, SEQ ID NO:588, and SEQ ID NO:589, respectively, and/or (ii) the variable light chain (VL) comprises CDR1, CDR2 and CDR3 according to SEQ ID NO:599, SEQ ID NO:601, SEQ ID NO:601, respectively; or (i) the variable heavy chain (VH) comprises CDR1, CDR2 and CDR3 according to SEQ ID NO:590, SEQ ID NO:591, and SEQ ID NO:592, respectively, and/or (ii) the variable light chain (VL) comprises CDR1, CDR2 and CDR3 according to SEQ ID NO:602, SEQ ID NO:603, SEQ ID NO:604, respectively; or (i) the variable heavy chain (VH) comprises CDR1, CDR2 and CDR3 according to SEQ ID NO:593, SEQ ID NO:594, and SEQ ID NO:595, respectively, and/or (ii) the variable light chain (VL) comprises CDR1, CDR2 and CDR3 according to SEQ ID NO:605, SEQ ID NO:606, SEQ ID NO:607, respectively, or SEQ ID NO: 608, SEQ ID NO: 609, SEQ ID NO: 610, respectively.

In some aspects described herein, the variable heavy chain comprises an amino acid sequence having at least 85% sequence identity to any one of SEQ ID NOs: 45-131 and 534-544. In some aspects described herein, the variable heavy chain comprises an amino acid sequence having at least 86% sequence identity to any one of SEQ ID NOs: 45-131 and 534-544. In some aspects described herein, the variable heavy chain comprises an amino acid sequence having at least 87% sequence identity to any one of SEQ ID NOs: 45-131 and 534-544. In some aspects described herein, the variable heavy chain comprises an amino acid sequence having at least 88% sequence identity to any one of SEQ ID NOs: 45-131 and 534-544. In some aspects described herein, the variable heavy chain comprises an amino acid sequence having at least 89% sequence identity to any one of SEQ ID NOs: 45-131 and 534-544. In some aspects described herein, the variable heavy chain comprises an amino acid sequence having at least 90% sequence identity to any one of SEQ ID NOs: 45-131 and 534-544. In some aspects described herein, the variable heavy chain comprises an amino acid sequence having at least 91% sequence identity to any one of SEQ ID NOs: 45-131 and 534-544. In some aspects described herein, the variable heavy chain comprises an amino acid sequence having at least 92% sequence identity to any one of SEQ ID NOs: 45-131 and 534-544. In some aspects described herein, the variable heavy chain comprises an amino acid sequence having at least 93% sequence identity to any one of SEQ ID NOs: 45-131 and 534-544. In some aspects described herein, the variable heavy chain comprises an amino acid sequence having at least 94% sequence identity to any one of SEQ ID NOs: 45-131 and 534-544. In some aspects described herein, the variable heavy chain comprises an amino acid sequence having at least 95% sequence identity to any one of SEQ ID NOs: 45-131 and 534-544. In some aspects described herein, the variable heavy chain comprises an amino acid sequence having at least 96% sequence identity to any one of SEQ ID NOs: 45-131 and 534-544. In some aspects described herein, the variable heavy chain comprises an amino acid sequence having at least 97% sequence identity to any one of SEQ ID NOs: 45-131 and 534-544. In some aspects described herein, the variable heavy chain comprises an amino acid sequence having at least 98% sequence identity to any one of SEQ ID NOs: 45-131 and 534-544. In some aspects described herein, the variable heavy chain comprises an amino acid sequence having at least 99% sequence identity to any one of SEQ ID NOs: 45-131 and 534-544. In some aspects described herein, the variable heavy chain comprises an amino acid sequence according to any one of SEQ ID NOs: 45-131 and 534-544.

In some aspects described herein, the variable light chain comprises an amino acid sequence having at least 85% sequence identity to any one of SEQ ID NOs: 132-227 and 545-558. In some aspects described herein, the variable light chain comprises an amino acid sequence having at least 86% sequence identity to any one of SEQ ID NOs: 132-227 and 545-558. In some aspects described herein, the variable light chain comprises an amino acid sequence having at least 87% sequence identity to any one of SEQ ID NOs: 132-227 and 545-558. In some aspects described herein, the variable light chain comprises an amino acid sequence having at least 88% sequence identity to any one of SEQ ID NOs: 132-227 and 545-558. In some aspects described herein, the variable light chain comprises an amino acid sequence having at least 89% sequence identity to any one of SEQ ID NOs: 132-227 and 545-558. In some aspects described herein, the variable light chain comprises an amino acid sequence having at least 90% sequence identity to any one of SEQ ID NOs: 132-227 and 545-558. In some aspects described herein, the variable light chain comprises an amino acid sequence having at least 91% sequence identity to any one of SEQ ID NOs: 132-227 and 545-558. In some aspects described herein, the variable light chain comprises an amino acid sequence having at least 92% sequence identity to any one of SEQ ID NOs: 132-227 and 545-558. In some aspects described herein, the variable light chain comprises an amino acid sequence having at least 93% sequence identity to any one of SEQ ID NOs: 132-227 and 545-558. In some aspects described herein, the variable light chain comprises an amino acid sequence having at least 94% sequence identity to any one of SEQ ID NOs: 132-227 and 545-558. In some aspects described herein, the variable light chain comprises an amino acid sequence having at least 95% sequence identity to any one of SEQ ID NOs: 132-227 and 545-558. In some aspects described herein, the variable light chain comprises an amino acid sequence having at least 96% sequence identity to any one of SEQ ID NOs: 132-227 and 545-558. In some aspects described herein, the variable light chain comprises an amino acid sequence having at least 97% sequence identity to any one of SEQ ID NOs: 132-227 and 545-558. In some aspects described herein, the variable light chain comprises an amino acid sequence having at least 98% sequence identity to any one of SEQ ID NOs: 132-227 and 545-558. In some aspects described herein, the variable light chain comprises an amino acid sequence having at least 99% sequence identity to any one of SEQ ID NOs: 132-227 and 545-558. In some aspects described herein, the variable light chain comprises an amino acid sequence according to any one of SEQ ID NOs: 132-227 and 545-558.

In some aspects described herein, the variable heavy chain comprises an amino acid sequence having any percent identity described above to any one of SEQ ID NOs: 45-131 and 534-544, and the variable light chain comprises an amino acid sequence having any percent identity described above to any one of SEQ ID NOs: 132-227 and 545-558. To illustrate, a non-limiting example pairing of variable heavy and light chains includes a variable heavy chain comprising an amino acid sequence having at least 88% sequence identity to any one of SEQ ID NOs: 45-131 and 534-544, and a variable light chain comprising an amino acid sequence having at least 93% sequence identity to any one of SEQ ID NOs: 132-227 and 545-558. In other aspects described herein, pairing of variable heavy and light chains includes a variable heavy chain comprising an amino acid sequence having any percent identity described above to any one of SEQ ID NOs: 45-131 and 534-544, and a variable light chain comprising an amino acid sequence according to any one of SEQ ID NOs: 132-227 and 545-558. In other aspects described herein, pairing of variable heavy and light chains includes a variable heavy chain comprising an amino acid sequence according to any one of SEQ ID NOs: 45-131 and 534-544, and a variable light chain comprising an amino acid sequence having any percent identity described above to any one of SEQ ID NOs: 132-227 and 545-558. To illustrate, a non-limiting example pairing of variable heavy and light chains includes a variable heavy chain comprising an amino acid sequence having at least 92% sequence identity to any one of SEQ ID NOs: 45-226 and a variable light chain comprising an amino acid sequence according to any one of SEQ ID NOs: 132-227 and 545-558. In yet other aspects described herein, the variable heavy chain comprises an amino acid sequence according to any one of SEQ ID NOs: 45-131 and 534-544, and the variable light chain comprises an amino acid sequence according to any one of SEQ ID NOs: 132-227 and 545-558.

In some aspects described herein, the CAR comprises, (i) the variable heavy chain (VH) comprising CDR1, CDR2 and CDR3 according to SEQ ID NO:10, SEQ ID NO:11, and SEQ ID NO:12, respectively, and/or (ii) the variable light chain (VL) comprises CDR1, CDR2 and CDR3 according to SEQ ID NO:13, SEQ ID NO:14, SEQ ID NO:15, respectively; or (i) the variable heavy chain (VH) comprises CDR1, CDR2 and CDR3 according to SEQ ID NO:370, SEQ ID NO:371, and SEQ ID NO:372, respectively, and/or (ii) the variable light chain (VL) comprises CDR1, CDR2 and CDR3 according to SEQ ID NO:418, SEQ ID NO:419, SEQ ID NO:420, respectively; or (i) the variable heavy chain (VH) comprises CDR1, CDR2 and CDR3 according to SEQ ID NO:373, SEQ ID NO:374, and SEQ ID NO:375 respectively, and/or (ii) the variable light chain (VL) comprises CDR1, CDR2 and CDR3 according to SEQ ID NO:421, SEQ ID NO:422, SEQ ID NO:423, respectively; or (i) the variable heavy chain (VH) comprises CDR1, CDR2 and CDR3 according to SEQ ID NO:376, SEQ ID NO:377, and SEQ ID NO:378, respectively, and/or (ii) the variable light chain (VL) comprises CDR1, CDR2 and CDR3 according to SEQ ID NO:424, SEQ ID NO:425, SEQ ID NO:426, respectively; or the variable heavy chain (VH) comprises CDR1, CDR2 and CDR3 according to SEQ ID NO:379, SEQ ID NO:380, and SEQ ID NO:381 respectively, and/or (ii) the variable light chain (VL) comprises CDR1, CDR2 and CDR3 according to SEQ ID NO:427, SEQ ID NO:428, SEQ ID NO:429, respectively; or (i) the variable heavy chain (VH) comprises CDR1, CDR2 and CDR3 according to SEQ ID NO:382, SEQ ID NO:383, and SEQ ID NO:384, respectively, and/or (ii) the variable light chain (VL) comprises CDR1, CDR2 and CDR3 according to SEQ ID NO:430, SEQ ID NO:431, SEQ ID NO:432, respectively; or (i) the variable heavy chain (VH) comprises CDR1, CDR2 and CDR3 according to SEQ ID NO:385, SEQ ID NO:386, and SEQ ID NO:387, respectively, and/or (ii) the variable light chain (VL) comprises CDR1, CDR2 and CDR3 according to SEQ ID NO:433, SEQ ID NO:434, SEQ ID NO:435, respectively; or (i) the variable heavy chain (VH) comprises CDR1, CDR2 and CDR3 according to SEQ ID NO:388, SEQ ID NO:389, and SEQ ID NO:390, respectively, and/or (ii) the variable light chain (VL) comprises CDR1, CDR2 and CDR3 according to SEQ ID NO:436, SEQ ID NO:437, SEQ ID NO:438, respectively; or (i) the variable heavy chain (VH) comprises CDR1, CDR2 and CDR3 according to SEQ ID NO:391, SEQ ID NO:392, and SEQ ID NO:393 respectively, and/or (ii) the variable light chain (VL) comprises CDR1, CDR2 and CDR3 according to SEQ ID NO:439, SEQ ID NO:440, SEQ ID NO:441, respectively; or (i) the variable heavy chain (VH) comprises CDR1, CDR2 and CDR3 according to SEQ ID NO:394, SEQ ID NO:395, and SEQ ID NO:396, respectively, and/or (ii) the variable light chain (VL) comprises CDR1, CDR2 and CDR3 according to SEQ ID NO:442, SEQ ID NO:443, SEQ ID NO:444, respectively; or (i) the variable heavy chain (VH) comprises CDR1, CDR2 and CDR3 according to SEQ ID NO:397, SEQ ID NO:398, and SEQ ID NO:399, respectively, and/or (ii) the variable light chain (VL) comprises CDR1, CDR2 and CDR3 according to SEQ ID NO:445, SEQ ID NO:446, SEQ ID NO:447, respectively; or (i) the variable heavy chain (VH) comprises CDR1, CDR2 and CDR3 according to SEQ ID NO:400, SEQ ID NO:401, and SEQ ID NO:402, respectively, and/or (ii) the variable light chain (VL) comprises CDR1, CDR2 and CDR3 according to SEQ ID NO:448, SEQ ID NO:449, SEQ ID NO:450, respectively; or (i) the variable heavy chain (VH) comprises CDR1, CDR2 and CDR3 according to SEQ ID NO:403, SEQ ID NO:404, and SEQ ID NO:405 respectively, and/or (ii) the variable light chain (VL) comprises CDR1, CDR2 and CDR3 according to SEQ ID NO:451, SEQ ID NO:452, SEQ ID NO:453, respectively; or (i) the variable heavy chain (VH) comprises CDR1, CDR2 and CDR3 according to SEQ ID NO:406, SEQ ID NO:407, and SEQ ID NO:408, respectively, and/or (ii) the variable light chain (VL) comprises CDR1, CDR2 and CDR3 according to SEQ ID NO:454, SEQ ID NO:455, SEQ ID NO:456, respectively; or (i) the variable heavy chain (VH) comprises CDR1, CDR2 and CDR3 according to SEQ ID NO:409, SEQ ID NO:410, and SEQ ID NO:411, respectively, and/or (ii) the variable light chain (VL) comprises CDR1, CDR2 and CDR3 according to SEQ ID NO:457, SEQ ID NO:458, SEQ ID NO:459, respectively; or (i) the variable heavy chain (VH) comprises CDR1, CDR2 and CDR3 according to SEQ ID NO:412, SEQ ID NO:413, and SEQ ID NO:414, respectively, and/or (ii) the variable light chain (VL) comprises CDR1, CDR2 and CDR3 according to SEQ ID NO:460, SEQ ID NO:461, SEQ ID NO:462, respectively; or (i) the variable heavy chain (VH) comprises CDR1, CDR2 and CDR3 according to SEQ ID NO:415, SEQ ID NO:416, and SEQ ID NO:417, respectively, and/or (ii) the variable light chain (VL) comprises CDR1, CDR2 and CDR3 according to SEQ ID NO:463, SEQ ID NO:464, SEQ ID NO:465, respectively; or (i) the variable heavy chain (VH) comprises CDR1, CDR2 and CDR3 according to SEQ ID NO:584, SEQ ID NO:585, and SEQ ID NO:586, respectively, and/or (ii) the variable light chain (VL) comprises CDR1, CDR2 and CDR3 according to SEQ ID NO:596, SEQ ID NO:597, SEQ ID NO:598, respectively; or (i) the variable heavy chain (VH) comprises CDR1, CDR2 and CDR3 according to SEQ ID NO:587, SEQ ID NO:588, and SEQ ID NO:589, respectively, and/or (ii) the variable light chain (VL) comprises CDR1, CDR2 and CDR3 according to SEQ ID NO:599, SEQ ID NO:601, SEQ ID NO:601, respectively; or (i) the variable heavy chain (VH) comprises CDR1, CDR2 and CDR3 according to SEQ ID NO:590, SEQ ID NO:591, and SEQ ID NO:592, respectively, and/or (ii) the variable light chain (VL) comprises CDR1, CDR2 and CDR3 according to SEQ ID NO:602, SEQ ID NO:603, SEQ ID NO:604, respectively; or (i) the variable heavy chain (VH) comprises CDR1, CDR2 and CDR3 according to SEQ ID NO:593, SEQ ID NO:594, and SEQ ID NO:595, respectively, and/or (ii) the variable light chain (VL) comprises CDR1, CDR2 and CDR3 according to SEQ ID NO:605, SEQ ID NO:606, SEQ ID NO:607, respectively, or SEQ ID NO: 608, SEQ ID NO: 609, SEQ ID NO: 610, respectively.

In some aspects described herein, the variable heavy chain comprises an amino acid sequence having at least 85% sequence identity to any one of SEQ ID NOs: 45-131 and 534-544. In some aspects described herein, the variable heavy chain comprises an amino acid sequence having at least 86% sequence identity to any one of SEQ ID NOs: 45-131 and 534-544. In some aspects described herein, the variable heavy chain comprises an amino acid sequence having at least 87% sequence identity to any one of SEQ ID NOs: 45-131 and 534-544. In some aspects described herein, the variable heavy chain comprises an amino acid sequence having at least 88% sequence identity to any one of SEQ ID NOs: 45-131 and 534-544. In some aspects described herein, the variable heavy chain comprises an amino acid sequence having at least 89% sequence identity to any one of SEQ ID NOs: 45-131 and 534-544. In some aspects described herein, the variable heavy chain comprises an amino acid sequence having at least 90% sequence identity to any one of SEQ ID NOs: 45-131 and 534-544. In some aspects described herein, the variable heavy chain comprises an amino acid sequence having at least 91% sequence identity to any one of SEQ ID NOs: 45-131 and 534-544. In some aspects described herein, the variable heavy chain comprises an amino acid sequence having at least 92% sequence identity to any one of SEQ ID NOs: 45-131 and 534-544. In some aspects described herein, the variable heavy chain comprises an amino acid sequence having at least 93% sequence identity to any one of SEQ ID NOs: 45-131 and 534-544. In some aspects described herein, the variable heavy chain comprises an amino acid sequence having at least 94% sequence identity to any one of SEQ ID NOs: 45-131 and 534-544. In some aspects described herein, the variable heavy chain comprises an amino acid sequence having at least 95% sequence identity to any one of SEQ ID NOs: 45-131 and 534-544. In some aspects described herein, the variable heavy chain comprises an amino acid sequence having at least 96% sequence identity to any one of SEQ ID NOs: 45-131 and 534-544. In some aspects described herein, the variable heavy chain comprises an amino acid sequence having at least 97% sequence identity to any one of SEQ ID NOs: 45-131 and 534-544. In some aspects described herein, the variable heavy chain comprises an amino acid sequence having at least 98% sequence identity to any one of SEQ ID NOs: 45-131 and 534-544. In some aspects described herein, the variable heavy chain comprises an amino acid sequence having at least 99% sequence identity to any one of SEQ ID NOs: 45-131 and 534-544. In some aspects described herein, the variable heavy chain comprises an amino acid sequence according to any one of SEQ ID NOs: 45-131 and 534-544.

In some aspects described herein, the variable light chain comprises an amino acid sequence having at least 85% sequence identity to any one of SEQ ID NOs: 132-227 and 545-558. In some aspects described herein, the variable light chain comprises an amino acid sequence having at least 86% sequence identity to any one of SEQ ID NOs: 132-227 and 545-558. In some aspects described herein, the variable light chain comprises an amino acid sequence having at least 87% sequence identity to any one of SEQ ID NOs: 132-227 and 545-558. In some aspects described herein, the variable light chain comprises an amino acid sequence having at least 88% sequence identity to any one of SEQ ID NOs: 132-227 and 545-558. In some aspects described herein, the variable light chain comprises an amino acid sequence having at least 89% sequence identity to any one of SEQ ID NOs: 132-227 and 545-558. In some aspects described herein, the variable light chain comprises an amino acid sequence having at least 90% sequence identity to any one of SEQ ID NOs: 132-227 and 545-558. In some aspects described herein, the variable light chain comprises an amino acid sequence having at least 91% sequence identity to any one of SEQ ID NOs: 132-227 and 545-558. In some aspects described herein, the variable light chain comprises an amino acid sequence having at least 92% sequence identity to any one of SEQ ID NOs: 132-227 and 545-558. In some aspects described herein, the variable light chain comprises an amino acid sequence having at least 93% sequence identity to any one of SEQ ID NOs: 132-227 and 545-558. In some aspects described herein, the variable light chain comprises an amino acid sequence having at least 94% sequence identity to any one of SEQ ID NOs: 132-227 and 545-558. In some aspects described herein, the variable light chain comprises an amino acid sequence having at least 95% sequence identity to any one of SEQ ID NOs: 132-227 and 545-558. In some aspects described herein, the variable light chain comprises an amino acid sequence having at least 96% sequence identity to any one of SEQ ID NOs: 132-227 and 545-558. In some aspects described herein, the variable light chain comprises an amino acid sequence having at least 97% sequence identity to any one of SEQ ID NOs: 132-227 and 545-558. In some aspects described herein, the variable light chain comprises an amino acid sequence having at least 98% sequence identity to any one of SEQ ID NOs: 132-227 and 545-558. In some aspects described herein, the variable light chain comprises an amino acid sequence having at least 99% sequence identity to any one of SEQ ID NOs: 132-227 and 545-558. In some aspects described herein, the variable light chain comprises an amino acid sequence according to any one of SEQ ID NOs: 132-227 and 545-558.

In some aspects described herein, the variable heavy chain comprises an amino acid sequence having any percent identity described above to any one of SEQ ID NOs: 45-131 and 534-544, and the variable light chain comprises an amino acid sequence having any percent identity described above to any one of SEQ ID NOs: 132-227 and 545-558. To illustrate, a non-limiting example pairing of variable heavy and light chains includes a variable heavy chain comprising an amino acid sequence having at least 88% sequence identity to any one of SEQ ID NOs: 45-131 and 534-544, and a variable light chain comprising an amino acid sequence having at least 93% sequence identity to any one of SEQ ID NOs: 132-227 and 545-558. In other aspects decribed herein, pairing of variable heavy and light chains includes a variable heavy chain comprising an amino acid sequence having any percent identity described above to any one of SEQ ID NOs: 45-131 and 534-544, and a variable light chain comprising an amino acid sequence according to any one of SEQ ID NOs: 132-227 and 545-558. In other aspects described herein, pairing of variable heavy and light chains includes a variable heavy chain comprising an amino acid sequence according to any one of SEQ ID NOs: 45-131 and 534-544, and a variable light chain comprising an amino acid sequence having any percent identity described above to any one of SEQ ID NOs: 132-227 and 545-558. To illustrate, a non-limiting example pairing of variable heavy and light chains includes a variable heavy chain comprising an amino acid sequence having at least 92% sequence identity to any one of SEQ ID NOs: 45-226 and a variable light chain comprising an amino acid sequence according to any one of SEQ ID NOs: 132-227 and 545-558. In yet other aspects described herein, the variable heavy chain comprises an amino acid sequence according to any one of SEQ ID NOs: 45-131 and 534-544, and the variable light chain comprises an amino acid sequence according to any one of SEQ ID NOs: 132-227 and 545-558.

In some aspects described herein, the CAR is capable of specifically binding to Vβ2 and, for example, comprises (i) a variable heavy chain (VH), and/or a variable light chain (VL). In some aspects, the Vβ2 CAR, for example comprises (i) a variable heavy chain (VH) according to SEQ ID NO: 45, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 46, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 47, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 48, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 49, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 50, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 51, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 52, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 53, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 54, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 55, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 56, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 57, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 58, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 59, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 60, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 61, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 62, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 63, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 64, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 65, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 66, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 67, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 68, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 69, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 70, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 71, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 72, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects, the CAR is capable of specifically binding to Vβ7.2 and, for example, comprises (i) a variable heavy chain (VH), and/or a variable light chain (VL). In some aspects, the Vβ7.2 CAR, for example comprises (i) a variable heavy chain (VH) according to SEQ ID NO: 73, and/or (ii) a variable light chain (VL) according to SEQ ID NO: 160; SEQ ID NO: 161; SEQ ID NO: 162; or SEQ ID NO: 163. In some aspects, the Vβ7.2 CAR, for example comprises (i) a variable heavy chain (VH) according to SEQ ID NO: 74, and/or (ii) a variable light chain (VL) according to SEQ ID NO: 160; SEQ ID NO: 161; SEQ ID NO: 162; or SEQ ID NO: 163. In some aspects, the Vβ7.2 CAR, for example comprises (i) a variable heavy chain (VH) according to SEQ ID NO: 75, and/or (ii) a variable light chain (VL) according to SEQ ID NO: 160; SEQ ID NO: 161; SEQ ID NO: 162; or SEQ ID NO: 163. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the CAR is capable of specifically binding to Vβ4 and, for example, comprises (i) a variable heavy chain (VH), and/or a variable light chain (VL). In some aspects, the Vβ4 CAR, for example comprises (i) a variable heavy chain (VH) according to SEQ ID NO: 76, and/or (ii) a variable light chain (VL) according to SEQ ID NO: 164; SEQ ID NO: 165; SEQ ID NO: 166; or SEQ ID NO: 167. In some aspects, the Vβ4 CAR, for example comprises (i) a variable heavy chain (VH) according to SEQ ID NO: 77, and/or (ii) a variable light chain (VL) according to SEQ ID NO: 164; SEQ ID NO: 165; SEQ ID NO: 166; or SEQ ID NO: 167. In some aspects, the Vβ4 CAR, for example comprises (i) a variable heavy chain (VH) according to SEQ ID NO: 78, and/or (ii) a variable light chain (VL) according to SEQ ID NO: 164; SEQ ID NO: 165; SEQ ID NO: 166; or SEQ ID NO: 167. In some aspects, the Vβ4 CAR, for example comprises (i) a variable heavy chain (VH) according to SEQ ID NO: 79, and/or (ii) a variable light chain (VL) according to SEQ ID NO: 164; SEQ ID NO: 165; SEQ ID NO: 166; or SEQ ID NO: 167. In some aspects, the Vβ4 CAR, for example comprises (i) a variable heavy chain (VH) according to SEQ ID NO: 80, and/or (ii) a variable light chain (VL) according to SEQ ID NO: 164; SEQ ID NO: 165; SEQ ID NO: 166; or SEQ ID NO: 167. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the CAR is capable of specifically binding to Vβ13.2 and, for example, comprises (i) a variable heavy chain (VH), and/or a variable light chain (VL). In some aspects, the Vβ13.2 CAR, for example comprises (i) a variable heavy chain (VH) according to SEQ ID NO: 81, and/or (ii) a variable light chain (VL) according to SEQ ID NO: 168; SEQ ID NO: 169; SEQ ID NO: 170; SEQ ID NO: 171; SEQ ID NO: 172; SEQ ID NO: 173; SEQ ID NO: 174; or SEQ ID NO: 175. In some aspects, the Vβ13.2 CAR, for example comprises (i) a variable heavy chain (VH) according to SEQ ID NO: 82, and/or (ii) a variable light chain (VL) according to SEQ ID NO: 168; SEQ ID NO: 169; SEQ ID NO: 170; SEQ ID NO: 171; SEQ ID NO: 172; SEQ ID NO: 173; SEQ ID NO: 174; or SEQ ID NO: 175. In some aspects, the Vβ13.2 CAR, for example comprises (i) a variable heavy chain (VH) according to SEQ ID NO: 83, and/or (ii) a variable light chain (VL) according to SEQ ID NO: 168; SEQ ID NO: 169; SEQ ID NO: 170; SEQ ID NO: 171; SEQ ID NO: 172; SEQ ID NO: 173; SEQ ID NO: 174; or SEQ ID NO: 175. In some aspects, the Vβ13.2 CAR, for example comprises (i) a variable heavy chain (VH) according to SEQ ID NO: 84, and/or (ii) a variable light chain (VL) according to SEQ ID NO: 168; SEQ ID NO: 169; SEQ ID NO: 170; SEQ ID NO: 171; SEQ ID NO: 172; SEQ ID NO: 173; SEQ ID NO: 174; or SEQ ID NO: 175. In some aspects, the Vβ13.2 CAR, for example comprises (i) a variable heavy chain (VH) according to SEQ ID NO: 85, and/or (ii) a variable light chain (VL) according to SEQ ID NO: 168; SEQ ID NO: 169; SEQ ID NO: 170; SEQ ID NO: 171; SEQ ID NO: 172; SEQ ID NO: 173; SEQ ID NO: 174; or SEQ ID NO: 175. In some aspects, the Vβ13.2 CAR, for example comprises (i) a variable heavy chain (VH) according to SEQ ID NO: 86, and/or (ii) a variable light chain (VL) according to SEQ ID NO: 168; SEQ ID NO: 169; SEQ ID NO: 170; SEQ ID NO: 171; SEQ ID NO: 172; SEQ ID NO: 173; SEQ ID NO: 174; or SEQ ID NO: 175. In some aspects, the Vβ13.2 CAR, for example comprises (i) a variable heavy chain (VH) according to SEQ ID NO: 87, and/or (ii) a variable light chain (VL) according to SEQ ID NO: 168; SEQ ID NO: 169; SEQ ID NO: 170; SEQ ID NO: 171; SEQ ID NO: 172; SEQ ID NO: 173; SEQ ID NO: 174; or SEQ ID NO: 175. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the CAR is capable of specifically binding to V814 and, for example, comprises (i) a variable heavy chain (VH), and/or a variable light chain (VL). In some aspects, the V814 CAR, for example comprises (i) a variable heavy chain (VH) according to SEQ ID NO: 88, and/or (ii) a variable light chain (VL) according to SEQ ID NO: 176; SEQ ID NO: 177; SEQ ID NO: 178; SEQ ID NO: 179; SEQ ID NO: 180; or SEQ ID NO: 181. In some aspects, the V814 CAR, for example comprises (i) a variable heavy chain (VH) according to SEQ ID NO: 89, and/or (ii) a variable light chain (VL) according to SEQ ID NO: 176; SEQ ID NO: 177; SEQ ID NO: 178; SEQ ID NO: 179; SEQ ID NO: 180; or SEQ ID NO: 181. In some aspects, the Vβ14 CAR, for example comprises (i) a variable heavy chain (VH) according to SEQ ID NO: 90, and/or (ii) a variable light chain (VL) according to SEQ ID NO: 176; SEQ ID NO: 177; SEQ ID NO: 178; SEQ ID NO: 179; SEQ ID NO: 180; or SEQ ID NO: 181. In some aspects, the Vβ14 CAR, for example comprises (i) a variable heavy chain (VH) according to SEQ ID NO: 91, and/or (ii) a variable light chain (VL) according to SEQ ID NO: 176; SEQ ID NO: 177; SEQ ID NO: 178; SEQ ID NO: 179; SEQ ID NO: 180; or SEQ ID NO: 181. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the CAR is capable of specifically binding to Vβ22 and, for example, comprises (i) a variable heavy chain (VH), and/or a variable light chain (VL). In some aspects, the Vβ22 CAR, for example comprises (i) a variable heavy chain (VH) according to SEQ ID NO: 92, and/or (ii) a variable light chain (VL) according to SEQ ID NO: 182; SEQ ID NO: 183; SEQ ID NO: 184; or SEQ ID NO: 185. In some aspects, the Vβ22 CAR, for example comprises (i) a variable heavy chain (VH) according to SEQ ID NO: 93, and/or (ii) a variable light chain (VL) according to SEQ ID NO: 182; SEQ ID NO: 183; SEQ ID NO: 184; or SEQ ID NO: 185. In some aspects, the Vβ22 CAR, for example comprises (i) a variable heavy chain (VH) according to SEQ ID NO: 94, and/or (ii) a variable light chain (VL) according to SEQ ID NO: 182; SEQ ID NO: 183; SEQ ID NO: 184; or SEQ ID NO: 185. In some aspects, the Vβ22 CAR, for example comprises (i) a variable heavy chain (VH) according to SEQ ID NO: 95, and/or (ii) a variable light chain (VL) according to SEQ ID NO: 182; SEQ ID NO: 183; SEQ ID NO: 184; or SEQ ID NO: 185. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the CAR is capable of specifically binding to Vβ11 and, for example, comprises (i) a variable heavy chain (VH), and/or a variable light chain (VL). In some aspects, the Vβ11 CAR, for example comprises (i) a variable heavy chain (VH) according to SEQ ID NO: 96, and/or (ii) a variable light chain (VL) according to SEQ ID NO: 186; SEQ ID NO: 187; SEQ ID NO: 188; or SEQ ID NO: 189. In some aspects, the Vβ11 CAR, for example comprises (i) a variable heavy chain (VH) according to SEQ ID NO: 97, and/or (ii) a variable light chain (VL) according to SEQ ID NO: 186; SEQ ID NO: 187; SEQ ID NO: 188; or SEQ ID NO: 189. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the CAR is capable of specifically binding to Vβ1 and, for example, comprises (i) a variable heavy chain (VH), and/or a variable light chain (VL). In some aspects, the Vβ1 CAR, for example comprises (i) a variable heavy chain (VH) according to SEQ ID NO: 98, and/or (ii) a variable light chain (VL) according to SEQ ID NO: 190; SEQ ID NO: 191; SEQ ID NO: 192; SEQ ID NO: 193; or SEQ ID NO: 194. In some aspects, the Vβ1 CAR, for example comprises (i) a variable heavy chain (VH) according to SEQ ID NO: 99, and/or (ii) a variable light chain (VL) according to SEQ ID NO: 190; SEQ ID NO: 191; SEQ ID NO: 192; SEQ ID NO: 193; or SEQ ID NO: 194. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the CAR is capable of specifically binding to Vβ12 and, for example, comprises (i) a variable heavy chain (VH), and/or a variable light chain (VL). In some aspects, the Vβ12 CAR, for example comprises (i) a variable heavy chain (VH) according to SEQ ID NO: 100, and/or (ii) a variable light chain (VL) according to SEQ ID NO: 195; SEQ ID NO: 196; SEQ ID NO: 197; SEQ ID NO: 198; or SEQ ID NO: 199. In some aspects, the Vβ12 CAR, for example comprises (i) a variable heavy chain (VH) according to SEQ ID NO: 101, and/or (ii) a variable light chain (VL) according to SEQ ID NO: 195; SEQ ID NO: 196; SEQ ID NO: 197; SEQ ID NO: 198; or SEQ ID NO: 199. In some aspects, the Vβ12 CAR, for example comprises (i) a variable heavy chain (VH) according to SEQ ID NO: 102, and/or (ii) a variable light chain (VL) according to SEQ ID NO: 195; SEQ ID NO: 196; SEQ ID NO: 197; SEQ ID NO: 198; or SEQ ID NO: 199, In some aspects, the Vβ12 CAR, for example comprises (i) a variable heavy chain (VH) according to SEQ ID NO: 103, and/or (ii) a variable light chain (VL) according to SEQ ID NO: 195; SEQ ID NO: 196; SEQ ID NO: 197; SEQ ID NO: 198; or SEQ ID NO: 199. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the CAR is capable of specifically binding to Vβ8 and, for example, comprises (i) a variable heavy chain (VH), and/or a variable light chain (VL). In some aspects, the Vβ8 CAR, for example comprises (i) a variable heavy chain (VH) according to SEQ ID NO: 104, and/or (ii) a variable light chain (VL) according to SEQ ID NO: 200; SEQ ID NO: 201; or SEQ ID NO: 202. In some aspects, the Vβ8 CAR, for example comprises (i) a variable heavy chain (VH) according to SEQ ID NO: 105, and/or (ii) a variable light chain (VL) according to SEQ ID NO: 200; SEQ ID NO: 201; or SEQ ID NO: 202. In some aspects, the Vβ8 CAR, for example comprises (i) a variable heavy chain (VH) according to SEQ ID NO: 106, and/or (ii) a variable light chain (VL) according to SEQ ID NO: 200; SEQ ID NO: 201; or SEQ ID NO: 202. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the CAR is capable of specifically binding to Vβ13.1 and, for example, comprises (i) a variable heavy chain (VH), and/or a variable light chain (VL). In some aspects, the Vβ13.1 CAR, for example comprises (i) a variable heavy chain (VH) according to SEQ ID NO: 107, and/or (ii) a variable light chain (VL) according to SEQ ID NO: 203; SEQ ID NO: 204; or SEQ ID NO: 205. In some aspects, the Vβ13.1 CAR, for example comprises (i) a variable heavy chain (VH) according to SEQ ID NO: 108, and/or (ii) a variable light chain (VL) according to SEQ ID NO: 203; SEQ ID NO: 204; or SEQ ID NO: 205. In some aspects, the Vβ13.1 CAR, for example comprises (i) a variable heavy chain (VH) according to SEQ ID NO: 109, and/or (ii) a variable light chain (VL) according to SEQ ID NO: 203; SEQ ID NO: 204; or SEQ ID NO: 205. In some aspects, the Vβ13.1 CAR, for example comprises (i) a variable heavy chain (VH) according to SEQ ID NO: 110, and/or (ii) a variable light chain (VL) according to SEQ ID NO: 203; SEQ ID NO: 204; or SEQ ID NO: 205. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the CAR is capable of specifically binding to Vβ5.1 and, for example, comprises (i) a variable heavy chain (VH), and/or a variable light chain (VL). In some aspects, the Vβ5.1 CAR, for example comprises (i) a variable heavy chain (VH) according to SEQ ID NO: 111, and/or (ii) a variable light chain (VL) according to SEQ ID NO: 206; SEQ ID NO: 207; or SEQ ID NO: 208. In some aspects, the Vβ5.1 CAR, for example comprises (i) a variable heavy chain (VH) according to SEQ ID NO: 112, and/or (ii) a variable light chain (VL) according to SEQ ID NO: 206; SEQ ID NO: 207; or SEQ ID NO: 208. In some aspects, the Vβ5.1 CAR, for example comprises (i) a variable heavy chain (VH) according to SEQ ID NO: 113, and/or (ii) a variable light chain (VL) according to SEQ ID NO: 206; SEQ ID NO: 207; or SEQ ID NO: 208. In some aspects, the Vβ5.1 CAR, for example comprises (i) a variable heavy chain (VH) according to SEQ ID NO: 114, and/or (ii) a variable light chain (VL) according to SEQ ID NO: 206; SEQ ID NO: 207; or SEQ ID NO: 208. In some aspects, the Vβ5.1 CAR, for example comprises (i) a variable heavy chain (VH) according to SEQ ID NO: 115, and/or (ii) a variable light chain (VL) according to SEQ ID NO: 206; SEQ ID NO: 207; or SEQ ID NO: 208. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the CAR is capable of specifically binding to Vβ9 and, for example, comprises (i) a variable heavy chain (VH), and/or a variable light chain (VL). In some aspects, the Vβ9 CAR, for example comprises (i) a variable heavy chain (VH) according to SEQ ID NO: 116, and/or (ii) a variable light chain (VL) according to SEQ ID NO: 209; SEQ ID NO: 210; SEQ ID NO: 211; or SEQ ID NO: 212. In some aspects, the Vβ9 CAR, for example comprises (i) a variable heavy chain (VH) according to SEQ ID NO: 117, and/or (ii) a variable light chain (VL) according to SEQ ID NO: 209; SEQ ID NO: 210; SEQ ID NO: 211; or SEQ ID NO: 212, In some aspects, the Vβ9 CAR, for example comprises (i) a variable heavy chain (VH) according to SEQ ID NO: 118, and/or (ii) a variable light chain (VL) according to SEQ ID NO: 209; SEQ ID NO: 210; SEQ ID NO: 211; or SEQ ID NO: 212. In some aspects, the Vβ9 CAR, for example comprises (i) a variable heavy chain (VH) according to SEQ ID NO: 119, and/or (ii) a variable light chain (VL) according to SEQ ID NO: 209; SEQ ID NO: 210; SEQ ID NO: 211; or SEQ ID NO: 212. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the CAR is capable of specifically binding to Vβ17 and, for example, comprises (i) a variable heavy chain (VH), and/or a variable light chain (VL). In some aspects, the Vβ17 CAR, for example comprises (i) a variable heavy chain (VH) according to SEQ ID NO: 120, and/or (ii) a variable light chain (VL) according to SEQ ID NO: 213; SEQ ID NO: 214; or SEQ ID NO: 215. In some aspects, the Vβ17 CAR, for example comprises (i) a variable heavy chain (VH) according to SEQ ID NO: 121, and/or (ii) a variable light chain (VL) according to SEQ ID NO: 213; SEQ ID NO: 214; or SEQ ID NO: 215. In some aspects, the Vβ17 CAR, for example comprises (i) a variable heavy chain (VH) according to SEQ ID NO: 122, and/or (ii) a variable light chain (VL) according to SEQ ID NO: 213; SEQ ID NO: 214; or SEQ ID NO: 215. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the CAR is capable of specifically binding to Vβ3 and, for example, comprises (i) a variable heavy chain (VH), and/or a variable light chain (VL). In some aspects, the Vβ3 CAR, for example comprises (i) a variable heavy chain (VH) according to SEQ ID NO: 123, and/or (ii) a variable light chain (VL) according to SEQ ID NO: 216; SEQ ID NO: 217; or SEQ ID NO: 218. In some aspects, the Vβ3 CAR, for example comprises (i) a variable heavy chain (VH) according to SEQ ID NO: 124, and/or (ii) a variable light chain (VL) according to SEQ ID NO: 216; SEQ ID NO: 217; or SEQ ID NO: 218. In some aspects, the Vβ3 CAR, for example comprises (i) a variable heavy chain (VH) according to SEQ ID NO: 125, and/or (ii) a variable light chain (VL) according to SEQ ID NO: 216; SEQ ID NO: 217; or SEQ ID NO: 218. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the CAR is capable of specifically binding to Vβ7.1 and, for example, comprises (i) a variable heavy chain (VH), and/or a variable light chain (VL). In some aspects, the Vβ7.1 CAR, for example comprises (i) a variable heavy chain (VH) according to SEQ ID NO: 126, and/or (ii) a variable light chain (VL) according to SEQ ID NO: 219; SEQ ID NO: 220; SEQ ID NO: 221; SEQ ID NO: 222; SEQ ID NO: 223; or SEQ ID NO: 224. In some aspects, the Vβ7.1 CAR, for example comprises (i) a variable heavy chain (VH) according to SEQ ID NO: 127, and/or (ii) a variable light chain (VL) according to SEQ ID NO: 219; SEQ ID NO: 220; SEQ ID NO: 221; SEQ ID NO: 222; SEQ ID NO: 223; or SEQ ID NO: 224, In some aspects, the Vβ7.1 CAR, for example comprises (i) a variable heavy chain (VH) according to SEQ ID NO: 128, and/or (ii) a variable light chain (VL) according to SEQ ID NO: 219; SEQ ID NO: 220; SEQ ID NO: 221; SEQ ID NO: 222; SEQ ID NO: 223; or SEQ ID NO: 224, In some aspects, the Vβ7.1 CAR, for example comprises (i) a variable heavy chain (VH) according to SEQ ID NO: 129, and/or (ii) a variable light chain (VL) according to SEQ ID NO: 219; SEQ ID NO: 220; SEQ ID NO: 221; SEQ ID NO: 222; SEQ ID NO: 223; or SEQ ID NO: 224, In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the CAR is capable of specifically binding to Vβ5.3 and, for example, comprises (i) a variable heavy chain (VH), and/or a variable light chain (VL). In some aspects, the Vβ5.3 CAR, for example comprises (i) a variable heavy chain (VH) according to SEQ ID NO: 130, and/or (ii) a variable light chain (VL) according to SEQ ID NO: 225; SEQ ID NO: 226; or SEQ ID NO: 227, In some aspects, the Vβ5.3 CAR, for example comprises (i) a variable heavy chain (VH) according to SEQ ID NO: 131, and/or (ii) a variable light chain (VL) according to SEQ ID NO: 225; SEQ ID NO: 226; or SEQ ID NO: 227. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the CAR is capable of specifically binding to Vβ20 and, for example, comprises (i) a variable heavy chain (VH), and/or a variable light chain (VL). In some aspects, the Vβ20 CAR, for example comprises (i) a variable heavy chain (VH) according to SEQ ID NO: 534, and/or (ii) a variable light chain (VL) according to SEQ ID NO: 545; SEQ ID NO: 546; or SEQ ID NO: 547. In some aspects, the Vβ20 CAR, for example comprises (i) a variable heavy chain (VH) according to SEQ ID NO: 535, and/or (ii) a variable light chain (VL) according to SEQ ID NO: 545; SEQ ID NO: 546; or SEQ ID NO: 547. In some aspects, the Vβ20 CAR, for example comprises (i) a variable heavy chain (VH) according to SEQ ID NO: 536, and/or (ii) a variable light chain (VL) according to SEQ ID NO: 545; SEQ ID NO: 546; or SEQ ID NO: 547. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the CAR is capable of specifically binding to Vβ18 and, for example, comprises (i) a variable heavy chain (VH), and/or a variable light chain (VL). In some aspects, the Vβ18 CAR, for example comprises (i) a variable heavy chain (VH) according to SEQ ID NO: 537, and/or (ii) a variable light chain (VL) according to SEQ ID NO: 548; SEQ ID NO: 549; or SEQ ID NO: 550. In some aspects, the Vβ18 CAR, for example comprises (i) a variable heavy chain (VH) according to SEQ ID NO: 538, and/or (ii) a variable light chain (VL) according to SEQ ID NO: 548; SEQ ID NO: 549; or SEQ ID NO: 550. In some aspects, the Vβ18 CAR, for example comprises (i) a variable heavy chain (VH) according to SEQ ID NO: 539, and/or (ii) a variable light chain (VL) according to SEQ ID NO: 548; SEQ ID NO: 549; or SEQ ID NO: 550. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the CAR is capable of specifically binding to Vβ13.6 and, for example, comprises (i) a variable heavy chain (VH), and/or a variable light chain (VL). In some aspects, the Vβ13.6 CAR, for example comprises (i) a variable heavy chain (VH) according to SEQ ID NO: 540, and/or (ii) a variable light chain (VL) according to SEQ ID NO: 551; or SEQ ID NO: 552. In some aspects, the Vβ13.6 CAR, for example comprises (i) a variable heavy chain (VH) according to SEQ ID NO: 541, and/or (ii) a variable light chain (VL) according to SEQ ID NO: 551; or SEQ ID NO: 552. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the CAR is capable of specifically binding to Vβ16 and, for example, comprises (i) a variable heavy chain (VH), and/or a variable light chain (VL). In some aspects, the Vβ16 CAR, for example comprises (i) a variable heavy chain (VH) according to SEQ ID NO: 542, and/or (ii) a variable light chain (VL) according to SEQ ID NO: 553; SEQ ID NO: 554; SEQ ID NO: 555; SEQ ID NO: 556; SEQ ID NO: 557; or SEQ ID NO: 558, In some aspects, the Vβ16 CAR, for example comprises (i) a variable heavy chain (VH) according to SEQ ID NO: 543, and/or (ii) a variable light chain (VL) according to SEQ ID NO: 553; SEQ ID NO: 554; SEQ ID NO: 555; SEQ ID NO: 556; SEQ ID NO: 557; or SEQ ID NO: 558, In some aspects, the Vβ16 CAR, for example comprises (i) a variable heavy chain (VH) according to SEQ ID NO: 544, and/or (ii) a variable light chain (VL) according to SEQ ID NO: 553; SEQ ID NO: 554; SEQ ID NO: 555; SEQ ID NO: 556; SEQ ID NO: 557; or SEQ ID NO: 558, In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

A variety of assays are known for identifying binding domains of the present disclosure that specifically bind with a particular target, including Western blot, ELISA, flow cytometry, or surface plasmon resonance analysis (e.g., using BIACORE analysis). The target can be an antigen of clinical interest against which it would be desirable to trigger an effector immune response that results in tumor killing. In some aspects as described herein, the target antigen of the binding domain of the chimeric antigen receptor can be, for example, a Vβ region of a TCR.

Illustrative ligand-binding domains include antigen binding proteins, such as antigen binding fragments of an antibody, such as scFv, extracellular domains of receptors, ligands for cell surface molecules/receptors, or receptor binding domains thereof, and tumor binding proteins. In some aspects described herein, the antigen binding domains, for example, included in a CAR of the disclosure can be, a variable region (Fv), a CDR, a Fab, an scFv, a VH, or VL.

In some aspects described herein, the binding domain of the CAR is a Vβ region of a TCR single chain antibody (scFv), and can be a murine, chimeric, human or humanized scFv. Single chain antibodies can be cloned from the V region genes of a hybridoma specific for a desired target. A technique which can be used for cloning the variable region heavy chain (VH) and variable region light chain (VL) has been disclosed, for example, in Orlandi et al., PNAS, 1989; 86: 3833-3837. Thus, in some aspects, a binding domain comprises an antibody-derived binding domain but can be a non-antibody derived binding domain. An antibody-derived binding domain can be a fragment of an antibody or a genetically engineered product of one or more fragments of the antibody, which fragment is involved in binding with the antigen.

In some aspects described herein, the TCR Vβ region is selected from the group consisting of Vβ1, Vβ2, Vβ3, Vβ4, Vβ5.1, Vβ5.3, Vβ6, Vβ7.1, Vβ7.2, Vβ8, Vβ9, Vβ10, Vβ11, Vβ12, Vβ13.1, Vβ13.2, Vβ14, Vβ15, Vβ16, Vβ17, Vβ18, Vβ19, Vβ20, Vβ21, Vβ22, Vβ23, Vβ24, Vβ25, Vβ26, Vβ27, Vβ28, Vβ29, Vβ30, VβA, VβB, and VβC. It will be understood that a combination of variable heavy and light chains described herein that specifically bind to the same TCR Vβ region are capable of providing increased in vitro and/or in vivo activity compared to a combination of variable heavy and light chains described herein that specifically bind to different TCR Vβ regions.

In some aspects described herein, the TCR Vβ region is selected from the group consisting of Vβ1, Vβ2, Vβ3, Vβ4, Vβ5.1, Vβ5.3, Vβ7.1, Vβ7.2, Vβ8, Vβ9, Vβ11, Vβ12, Vβ13.1, Vβ13.2, Vβ14, Vβ17, and Vβ22.

In some aspects described herein, the TCR Vβ region is Vβ2.

In some aspects described herein, the antigen-binding domain is a scFv. In some aspects described herein, the scFv for example, comprises an amino acid sequence according to any of SEQ ID NOs: 16-44.

In some aspects, the CARs of the present disclosure can comprise a linker between one or more domains, e.g., added for appropriate spacing and conformation of the molecule. For example, in some aspects, there may be a linker between the binding domain VH or VL. In some aspects, the linker can be between 1-10 amino acids long. In some aspects, the linker between any of the domains of the chimeric antigen receptor can be between 1-20 or 20 amino acids long. In this regard, the linker can be 1, 2, 3, 4, 5, 6, 7, 8, 9, 10, 11, 12, 13, 14, 15, 16, 17, 18, 19 or 20 amino acids long. In some aspects, the linker can be 21, 22, 23, 24, 25, 26, 27, 28, 29 or 30 amino acids long. Ranges including the numbers described herein are also included herein, e.g., a linker 10-30 amino acids long.

In some aspects, linkers suitable for use in the CAR disclosed herein are flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

Exemplary flexible linkers can include glycine polymers (G)n, glycine-serine polymers, where n is an integer of at least one, glycine-alanine polymers, alanine-serine polymers, and other flexible linkers known in the art. Glycine and glycine-serine polymers are relatively unstructured, and therefore can be able to serve as a neutral tether between domains of fusion proteins such as the CARs disclosed herein. Glycine accesses significantly more phi-psi space than even alanine, and is much less restricted than residues with longer side chains (see Scheraga, Rev. Computational Chem. 11173-142 (1992)). The ordinarily skilled artisan will recognize that design of a CAR can include linkers that are all or partially flexible, such that the linker can include a flexible linker as well as one or more portions that confer less flexible structure to provide for a desired CAR structure.

In some aspects described herein, the scFv comprises a linker peptide. In some aspects described herein, the scFv comprises a linker peptide according to SEQ ID NO: 4.

Signals generated through the T cell receptor alone are generally insufficient for full activation of a T cell, thus a costimulatory signal or signaling domain can be present to activate the T cell. Examples of signal transducing domains for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. In some cases, signaling domains comprise two distinct classes of cytoplasmic signaling sequences, those that initiate antigen-dependent primary activation, and those that act in an antigen-independent manner to provide a secondary or co-stimulatory signal. Primary cytoplasmic signaling sequences can comprise signaling motifs that are known as immunoreceptor tyrosine-based activation motifs (ITAMs). ITAMs can be found in the intracytoplasmic tail of a variety of receptors that serve as binding sites for syk/zap70 class tyrosine kinases.

In some aspects described herein, the ITAMS, for example can include those derived from TCRzeta, FcRgamma, FcRbeta, FcRepsilon, CD3gamma, CD3delta, CD3epsilon, CD3zeta, CD5, CD22, CD79a, CD79b and CD66d. In some aspects, the signal transducing domain of the CAR can comprise the CD3zeta/CD247 signaling domain.

In some aspects, the CAR disclosed herein can comprise a costimulatory domain, e.g., derived from a costimulatory molecule. Costimulatory molecules can include, but are not limited to, an MHC class I molecule, BTLA and Toll ligand receptor. Examples of costimulatory molecules include, e.g., CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like. A costimulatory molecule can be a cell surface molecule other than an antigen receptor or their ligands that can contribute to an efficient immune response.

Accordingly, while the present disclosure provides exemplary costimulatory domains derived from 4-1BB, other costimulatory domains are contemplated for use with the CARs disclosed herein. The inclusion of one or more co-stimulatory signaling domains can enhance the efficacy and expansion of T cells expressing CAR receptors disclosed herein. The intracellular signaling and costimulatory signaling domains can be linked in any order in tandem to the carboxyl terminus of the transmembrane domain.

Although scFv-based CARs engineered to contain a signaling domain from CD3 or FcRgamma have been shown to deliver a potent signal for T cell activation and effector function, they are not sufficient to elicit signals that promote T cell survival and expansion in the absence of a concomitant costimulatory signal. Other CARs containing a binding domain, a hinge, a transmembrane and the signaling domain derived from CD3zeta or FcRgamma together with one or more costimulatory signaling domains (e.g., intracellular costimulatory domains derived from CD28, CD137, CD134 and CD278) can more effectively direct antitumor activity as well as increased cytokine secretion, lytic activity, survival and proliferation in CAR expressing T cells in vitro, and in animal models and cancer patients (Milone et al., Molecular Therapy, 2009; 17: 1453-1464; Zhong et al., Molecular Therapy, 2010; 18: 413-420; Carpenito et al., PNAS, 2009; 106:3360-3365).

In some aspects, the Vβ region of a TCR-binding CARs of the disclosure comprises (a) an anti-Vβ region of a TCR scFv as a binding domain (e.g., an scFv having binding regions (e.g., CDRs or variable domains) from an anti-Vβ region of a TCR antibody or antigen-binding fragment thereof disclosed herein); (b) a hinge region (e.g., derived from human CD8α); (c) a transmembrane domain (e.g., a human CD28α transmembrane domain); and (d) a signaling domain (e.g., a human T cell receptor CD3zeta chain signaling domain), and optionally one or more costimulatory signaling domains, e.g., a 4-1BB co-stimulatory domain.

In some aspects, the different protein domains are arranged from amino to carboxyl terminus in the following order: binding domain, hinge and transmembrane domain. The intracellular signaling domain and optional co-stimulatory signaling domains are linked to the transmembrane carboxy terminus in any order in tandem to form a single chain chimeric polypeptide.

In some aspects, a nucleic acid construct encoding a Vβ region of a TCR-binding CAR is a chimeric nucleic acid molecule comprising different coding sequences, for example, (5′ to 3′) the coding sequences of a human anti-Vβ region of a TCR scFv, a human CD8α-hinge region, a human CD28α transmembrane domain and a CD247 signaling domain. In some aspects, a nucleic acid construct encoding a Vβ region of a TCR-binding CAR is a chimeric nucleic acid molecule comprising different coding sequences, for example, (5′ to 3′) the coding sequences of a human anti-Vβ region of a TCR scFv, a human CD8α-hinge, a human CD28α transmembrane domain, a 4-1BB co-stimulatory domain, and a CD247 signaling domain.

In some aspects described herein, the polynucleotide encodes a CAR disclosed herein, and is inserted into a vector. In some aspects, the vector as used herein is a vehicle into which a polynucleotide encoding a protein can be covalently inserted so as to bring about the expression of that protein and/or the cloning of the polynucleotide. The isolated polynucleotide can be inserted into a vector using any suitable methods known in the art, for example, without limitation, the vector can be digested using appropriate restriction enzymes and then can be ligated with the isolated polynucleotide having matching restriction ends. In some aspects, expression vectors have the ability to incorporate and express heterologous or modified nucleic acid sequences coding for at least part of a gene product capable of being transcribed in a cell. In most cases, RNA molecules are then translated into a protein. Expression vectors can contain a variety of control sequences, which refer to nucleic acid sequences for the transcription and possibly translation of an operatively linked coding sequence in a particular host organism. In addition to control sequences that govern transcription and translation, vectors and expression vectors can contain nucleic acid sequences that serve other functions as well and are discussed infra. An expression vector can comprise additional elements, for example, the expression vector can have two replication systems, thus allowing it to be maintained in two organisms, for example in human cells for expression and in a prokaryotic host for cloning and amplification.

The expression vector can have 5′ upstream and 3′ downstream regulatory elements such as promoter sequences (e.g., CMV, PGK or EF1alpha promoters), ribosome recognition and binding TATA box, and 3′ UTR AAUAAA transcription termination sequence for the efficient gene transcription and translation in its respective host cell. Other suitable promoters can include the constitutive promoter of simian virus 40 (SV40) early promoter, mouse mammary tumor virus (MMTV), HIV LTR promoter, MoMuLV promoter, avian leukemia virus promoter, EBV immediate early promoter, and rous sarcoma virus promoter. Human gene promoters can also be used, including, but not limited to the actin promoter, the myosin promoter, the hemoglobin promoter, and the creatine kinase promoter. In some aspects, inducible promoters are also contemplated as part of the vectors expressing chimeric antigen receptor. This provides a molecular switch capable of turning on expression of the polynucleotide sequence of interest or turning off expression. Examples of inducible promoters include, but are not limited to a metallothionine promoter, a glucocorticoid promoter, a progesterone promoter, or a tetracycline promoter.

The expression vector can have additional sequence such as 6×-histidine, c-Myc, and FLAG tags that are incorporated into the expressed CARs. Thus, the expression vector can be engineered to contain 5′ and 3′ untranslated regulatory sequences that sometimes can function as enhancer sequences, promoter regions and/or terminator sequences that can facilitate or enhance efficient transcription of the nucleic acid(s) of interest carried on the expression vector. An expression vector can also be engineered for replication and/or expression functionality (e.g., transcription and translation) in a particular cell type, cell location, or tissue type. Expression vectors can include a selectable marker for maintenance of the vector in the host or recipient cell.

In some aspects, the vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are Lenti-X™ Bicistronic Expression System (Neo) vectors (Clontrch), pClneo vectors (Promega) for expression in mammalian cells; pLenti4/V5-DEST™ pLenti6/V5-DEST™, and pLenti6.2N5-GW/lacZ (Invitrogen) for lentivirus-mediated gene transfer and expression in mammalian cells. The coding sequences of the CARs disclosed herein can be ligated into such expression vectors for the expression of the chimeric protein in mammalian cells.

In some aspects, for expression of a Vβ region of a TCR-binding CAR, the vector can be introduced into a host cell to allow expression of the polypeptide within the host cell. The expression vectors can contain a variety of elements for controlling expression, including without limitation, promoter sequences, transcription initiation sequences, enhancer sequences, selectable markers, and signal sequences. These elements can be selected as appropriate by a person of ordinary skill in the art, as disclosed herein. For example, the promoter sequences can be selected to promote the transcription of the polynucleotide in the vector. Suitable promoter sequences include, without limitation, T7 promoter, T3 promoter, SP6 promoter, beta-actin promoter, EF1a promoter, CMV promoter, and SV40 promoter. Enhancer sequences can be selected to enhance the transcription of the polynucleotide. Selectable markers can be selected to allow selection of the host cells inserted with the vector from those not, for example, the selectable markers can be genes that confer antibiotic resistance. Signal sequences can be selected to allow the expressed polypeptide to be transported outside of the host cell.

In some aspects described herein, a nucleic acid molecule encoding the VH of the chimeric antigen receptor is disclosed. In aspects described herein, the nucleic acid molecule encodes a polypeptide comprising the VH of the chimeric antigen receptor of any of SEQ ID NO:45-131 and 534-544.

In some aspects described herein, a nucleic acid molecule encoding the VL of the chimeric antigen receptor is disclosed. In aspects described herein, the nucleic acid molecule encodes a polypeptide comprising the VL of the chimeric antigen receptor of any of SEQ ID NO:132-227 and 545-558.

In some aspects described herein, a nucleic acid molecule encoding the chimeric antigen receptor is disclosed. In aspects described herein, the nucleic acid molecule encoding the chimeric antigen receptor of any of SEQ ID NO:16-44.

In some aspects described herein, a vector comprising the isolated polynucleotide is disclosed. In some aspects described herein, the vector is selected from the group consisting of a retroviral vector, an adenoviral vector, a lentiviral vector, and an adeno-associated viral vector.

For cloning of the polynucleotide, the vector can be introduced into a host cell (an isolated host cell) to allow replication of the vector itself and thereby amplify the copies of the polynucleotide contained therein. The cloning vectors can contain sequence components generally include, without limitation, an origin of replication, promoter sequences, transcription initiation sequences, enhancer sequences, and selectable markers. These elements can be selected as appropriate by a person of ordinary skill in the art. For example, the origin of replication can be selected to promote autonomous replication of the vector in the host cell.

In some aspects, the present disclosure provides isolated host cells containing the vectors provided herein. The host cells containing the vector can be useful in expression or cloning of the polynucleotide contained in the vector. Suitable host cells can include, without limitation, prokaryotic cells, fungal cells, yeast cells, or higher eukaryotic cells such as mammalian cells. Suitable prokaryotic cells for this purpose include, without limitation, eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

The CARs of the present disclosure can be introduced into a host cell using transfection and/or transduction techniques known in the art. As used herein, the terms, “transfection,” and, “transduction,” refer to the processes by which an exogenous nucleic acid sequence is introduced into a host cell. The nucleic acid can be integrated into the host cell DNA or can be maintained extrachromosomally. The nucleic acid can be maintained transiently or can be a stable introduction. Transfection can be accomplished by a variety of means known in the art including but not limited to calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Transduction refers to the delivery of a gene(s) using a viral or retroviral vector by means of viral infection rather than by transfection. In some aspects, retroviral vectors are transduced by packaging the vectors into virions prior to contact with a cell. For example, a nucleic acid encoding a Vβ region of a TCR CAR carried by a retroviral vector can be transduced into a cell through infection and pro virus integration.

The immune effector cells, such as T cells, can be genetically modified following isolation using known methods, or the immune effector cells can be activated and expanded (or differentiated in the case of progenitors) in vitro prior to being genetically modified. In another aspects, the immune effector cells, such as T cells, are genetically modified with the chimeric antigen receptors disclosed herein (e.g., transduced with a viral vector comprising a nucleic acid encoding a CAR) and then are activated and expanded in vitro. Methods for activating and expanding T cells are known in the art and are disclosed, for example, in U.S. Pat. Nos. 6,905,874; 6,867,041; 6,797,514; WO2012079000. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2.

Anti-CD3 and anti-CD28 antibodies attached to the same bead serve as a “surrogate” antigen presenting cell (APC). In some aspects, the T cells can be activated and stimulated to proliferate with feeder cells and appropriate antibodies and cytokines using methods such as those disclosed in U.S. Pat. Nos. 6,040,177; 5,827,642; and WO2012129514.

CAR-expressing immune effector cells prepared as disclosed herein can be utilized in methods and compositions for adoptive immunotherapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg.

In some aspects, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin.

The cells can be autologous or heterologous to the patient undergoing therapy. If desired, the treatment can also include administration of mitogens (e.g., PHA) or lymphokines, cytokines, and/or chemokines (e.g., IFN-γ, IL-2, IL-12, TNF-α, IL-18, and TNF-β, GM-CSF, IL-4, IL-13, Flt3-L, RANTES, MIP1α, etc.) as disclosed herein to enhance induction of the immune response.

The present disclosure provides methods for making the immune effector cells that express the CAR as disclosed herein. In some aspects, the immune effector cells are isolated from an individual and genetically modified without further manipulation in vitro. Such cells can then be directly re-administered into the individual. In some aspects, the immune effector cells are first activated and stimulated to proliferate in vitro prior to being genetically modified to express a CAR. In this regard, the immune effector cells can be cultured before or after being genetically modified (i.e., transduced or transfected to express a CAR as disclosed herein).

Prior to in vitro manipulation or genetic modification of the immune effector cells disclosed herein, the source of cells can be obtained from a subject. In particular, the immune effector cells for use with the CARs as disclosed herein comprise T cells. T cells can be obtained from a number of sources, including peripheral blood mononuclear cells, bone marrow, lymph nodes tissue, cord blood, thymus issue, tissue from a site of infection, ascites, pleural effusion, spleen tissue, and tumors. In some aspects, T cells can be obtained from a unit of blood collected from the subject using any number of techniques known to the skilled person, such as an exemplerary FICOLL separation. In some aspects, cells from the circulating blood of an individual are obtained by apheresis. The apheresis product typically contains lymphocytes, including T cells, monocytes, granulocyte, B cells, other nucleated white blood cells, red blood cells, and platelets.

In some aspects, the cells collected by apheresis can be washed to remove the plasma fraction and to place the cells in an appropriate buffer or media for subsequent processing. In some aspects, the cells are washed with PBS. In some aspects, the washed solution lacks calcium, and can lack magnesium or can lack many, if not all, divalent cations. As would be appreciated by those of ordinary skill in the art, a washing step can be accomplished by methods known to those in the art, such as by using a semiautomated flowthrough centrifuge. After washing, the cells can be resuspended in a variety of biocompatible buffers or other saline solution with or without buffer. In some aspects, the undesirable components of the apheresis sample can be removed in the cell directly resuspended culture media.

In some aspects, T cells are isolated from peripheral blood mononuclear cells (PBMCs) by lysing the red blood cells and depleting the monocytes, for example, by centrifugation through a PERCOLL™ gradient. A specific subpopulation of T cells, such as CD28+, CD4+, CD8+, CD45RA+, and CD45RO+ T cells, can be further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD1b, CD16, HLA-DR, and CD8. Flow cytometry and cell sorting can also be used to isolate cell populations of interest for use in the present disclosure.

PBMCs can be used directly for genetic modification with the CARs using methods as disclosed herein. In some aspects, after isolation of PBMC, T lymphocytes are further isolated and in some aspects, both cytotoxic and helper T lymphocytes can be sorted into naive, memory, and effector T cell subpopulations either before or after genetic modification and/or expansion. CD8+ cells can be obtained by using standard methods. In some aspects, CD8+ cells are further sorted into naive, central memory, and effector cells by identifying cell surface antigens that are associated with each of those types of CD8+ cells.

The CAR expressing immune effector cell populations of the present disclosure can be administered either alone, or as a pharmaceutical composition in combination with diluents and/or with other components such as IL-2 or other cytokines or cell populations. Briefly, pharmaceutical compositions of the present disclosure can comprise a CAR-expressing immune effector cell population, such as T cells, as disclosed herein, in combination with one or more pharmaceutically or physiologically acceptable carriers, diluents or excipients. Such compositions comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure can be formulated for intravenous administration.

Humoral immune responses, mediated primarily by helper T cells capable of activating B cells thus leading to antibody production, may be induced. A variety of techniques may be used for analyzing the type of immune responses induced by the compositions of the present disclosure, which are well disclosed in the art; e.g., Current Protocols in Immunology, Edited by: John E. Coligan, Ada M. Kruisbeek, David H. Margulies, Ethan M. Shevach, Warren Strober (2001) John Wiley & Sons, N.Y., N.Y.

The methods for administering the cell compositions disclosed herein includes any method which is effective to result in reintroduction of ex vivo genetically modified immune effector cells that either directly express a CAR of the disclosure in the subject or on reintroduction of the genetically modified progenitors of immune effector cells that on introduction into a subject differentiate into mature immune effector cells that express the CAR. One method comprises transducing peripheral blood T cells ex vivo with a nucleic acid construct in accordance with the disclosure and returning the transduced cells into the subject.

The present disclosure also provides a vector in which a nucleic acid molecule encoding the CAR of the present disclosure is inserted. Vectors, including those derived from retroviruses such as lentivirus, are suitable tools to achieve long-term gene transfer since they allow long-term, stable integration of a transgene and its propagation in daughter cells. Lentiviral vectors have the added advantage over vectors derived from onco-retroviruses, such as murine leukemia viruses, in that they can transduce non-proliferating cells, such as hepatocytes. They also have the added advantage of resulting in low immunogenicity in the subject into which they are introduced.

Prior to in vitro manipulation or genetic modification of the immune effector cells disclosed herein, the source of cells can be obtained from a subject. In particular, the immune effector cells for use with the CARs as disclosed herein comprise T cells. T cells can be obtained from a number of sources, including peripheral blood mononuclear cells, bone marrow, lymph nodes tissue, cord blood, thymus issue, tissue from a site of infection, ascites, pleural effusion, spleen tissue, and tumors.

### II. Anti-TCRVβ Antibodies and Antigen-Binding Fragments Thereof

In some aspects, provided herein are antibodies (e.g. monoclonal antibodies) and antigen-binding fragments thereof which specifically bind to Vβ region of a TCR (e.g., human Vβ region of a TCR).

In some aspects described herein, the antibody or antigen-binding fragment, for example, comprises (i) a variable heavy chain (VH) which comprises: (a) CDR1, CDR2 and CDR3 according to SEQ ID NO:10, SEQ ID NO:11, and SEQ ID NO:12, respectively; or (b) CDR1, CDR2 and CDR3 according to SEQ ID NO:370, SEQ ID NO:371, and SEQ ID NO:372, respectively; or (c) CDR1, CDR2 and CDR3 according to SEQ ID NO:373, SEQ ID NO:374, and SEQ ID NO:375 respectively; or (d) CDR1, CDR2 and CDR3 according to SEQ ID NO:376, SEQ ID NO:377, and SEQ ID NO:378, respectively; or (e) CDR1, CDR2 and CDR3 according to SEQ ID NO:379, SEQ ID NO:380, and SEQ ID NO:381 respectively; or (f) CDR1, CDR2 and CDR3 according to SEQ ID NO:382, SEQ ID NO:383, and SEQ ID NO:384, respectively; or (g) CDR1, CDR2 and CDR3 according to SEQ ID NO:385, SEQ ID NO:386, and SEQ ID NO:387, respectively; or (h) CDR1, CDR2 and CDR3 according to SEQ ID NO:388, SEQ ID NO:389, and SEQ ID NO:390, respectively; or (i) CDR1, CDR2 and CDR3 according to SEQ ID NO:391, SEQ ID NO:392, and SEQ ID NO:393, respectively; or (j) CDR1, CDR2 and CDR3 according to SEQ ID NO:394, SEQ ID NO:395, and SEQ ID NO:396, respectively; or (k) CDR1, CDR2 and CDR3 according to SEQ ID NO:397, SEQ ID NO:398, and SEQ ID NO:399, respectively; or (l) CDR1, CDR2 and CDR3 according to SEQ ID NO:400, SEQ ID NO:401, and SEQ ID NO:402, respectively; or (m) CDR1, CDR2 and CDR3 according to SEQ ID NO:403, SEQ ID NO:404, and SEQ ID NO:405, respectively; or (n) CDR1, CDR2 and CDR3 according to SEQ ID NO:406, SEQ ID NO:407, and SEQ ID NO:408, respectively; or (o) CDR1, CDR2 and CDR3 according to SEQ ID NO:409, SEQ ID NO:410, and SEQ ID NO:411, respectively; or (p) CDR1, CDR2 and CDR3 according to SEQ ID NO:412, SEQ ID NO:413, and SEQ ID NO:414, respectively; or (q) CDR1, CDR2 and CDR3 according to SEQ ID NO:415, SEQ ID NO:416, and SEQ ID NO:417, respectively; or (r) CDR1, CDR2 and CDR3 according to SEQ ID NO:584, SEQ ID NO:585, and SEQ ID NO:586, respectively; or (s) CDR1, CDR2 and CDR3 according to SEQ ID NO:587, SEQ ID NO:588, and SEQ ID NO:589, respectively; or (t) CDR1, CDR2 and CDR3 according to SEQ ID NO:590, SEQ ID NO:591, and SEQ ID NO:592, respectively; or (u) CDR1, CDR2 and CDR3 according to SEQ ID NO:593, SEQ ID NO:594, and SEQ ID NO:595, respectively; and/or (ii) a variable light chain (VL) which comprises: (a) CDR1, CDR2 and CDR3 according to SEQ ID NO:13, SEQ ID NO:14, SEQ ID NO:15, respectively; or (b) CDR1, CDR2 and CDR3 according to SEQ ID NO:418, SEQ ID NO:419, SEQ ID NO:420, respectively; or (c) CDR1, CDR2 and CDR3 according to SEQ ID NO:421, SEQ ID NO:422, SEQ ID NO:423, respectively; or (d) CDR1, CDR2 and CDR3 according to SEQ ID NO:424, SEQ ID NO:425, SEQ ID NO:426, respectively; or (e) CDR1, CDR2 and CDR3 according to SEQ ID NO:427, SEQ ID NO:428, SEQ ID NO:429, respectively; or (f) CDR1, CDR2 and CDR3 according to SEQ ID NO:430, SEQ ID NO:431, SEQ ID NO:432, respectively; or (g) CDR1, CDR2 and CDR3 according to SEQ ID NO:433, SEQ ID NO:434, SEQ ID NO:435, respectively; or (h) CDR1, CDR2 and CDR3 according to SEQ ID NO:436, SEQ ID NO:437, SEQ ID NO:438, respectively; or (i) CDR1, CDR2 and CDR3 according to SEQ ID NO:439, SEQ ID NO:440, SEQ ID NO:441, respectively; or (j) CDR1, CDR2 and CDR3 according to SEQ ID NO:442, SEQ ID NO:443, SEQ ID NO:444, respectively; or (k) CDR1, CDR2 and CDR3 according to SEQ ID NO:445, SEQ ID NO:446, SEQ ID NO:447, respectively; or (1) CDR1, CDR2 and CDR3 according to SEQ ID NO:448, SEQ ID NO:449, SEQ ID NO:450, respectively; or (m) CDR1, CDR2 and CDR3 according to SEQ ID NO:451, SEQ ID NO:452, SEQ ID NO:453, respectively; or (n) CDR1, CDR2 and CDR3 according to SEQ ID NO:454, SEQ ID NO:455, SEQ ID NO:456, respectively; or (o) CDR1, CDR2 and CDR3 according to SEQ ID NO:457, SEQ ID NO:458, SEQ ID NO:459, respectively; or (p) CDR1, CDR2 and CDR3 according to SEQ ID NO:460, SEQ ID NO:461, SEQ ID NO:462, respectively; or (q) CDR1, CDR2 and CDR3 according to SEQ ID NO:463, SEQ ID NO:464, SEQ ID NO:465, respectively; or (r) CDR1, CDR2 and CDR3 according to SEQ ID NO:596, SEQ ID NO:597, SEQ ID NO:598, respectively; or (s) CDR1, CDR2 and CDR3 according to SEQ ID NO:599, SEQ ID NO:601, SEQ ID NO:601, respectively; or (t) CDR1, CDR2 and CDR3 according to SEQ ID NO:602, SEQ ID NO:603, SEQ ID NO:604, respectively; or (u) CDR1, CDR2 and CDR3 according to SEQ ID NO:605, SEQ ID NO:606, SEQ ID NO:607, respectively, or SEQ ID NO: 608, SEQ ID NO: 609, SEQ ID NO: 610, respectively.

In some aspects described herein, an antibody or antigen-binding fragment thereof, is capable of specifically binding to a TCR Vβ region, comprising a heavy chain variable region (VH) having an amino acid sequence according to any of SEQ ID NOs: 228-295 and 559-569. In some aspects described herein, the variable heavy chain comprises an amino acid sequence having at least 85% sequence identity to any one of SEQ ID NOs: 228-295 and 559-569. In some aspects described herein, the variable heavy chain comprises an amino acid sequence having at least 86% sequence identity to any one of SEQ ID NOs: 228-295 and 559-569. In some aspects described herein, the variable heavy chain comprises an amino acid sequence having at least 87% sequence identity to any one of SEQ ID NOs: 228-295 and 559-569. In some aspects described herein, the variable heavy chain comprises an amino acid sequence having at least 88% sequence identity to any one of SEQ ID NOs: 228-295 and 559-569. In some aspects described herein, the variable heavy chain comprises an amino acid sequence having at least 89% sequence identity to any one of SEQ ID NOs: 228-295 and 559-569. In some aspects described herein, the variable heavy chain comprises an amino acid sequence having at least 90% sequence identity to any one of SEQ ID NOs: 228-295 and 559-569. In some aspects described herein, the variable heavy chain comprises an amino acid sequence having at least 91% sequence identity to any one of SEQ ID NOs: 228-295 and 559-569. In some aspects described herein, the variable heavy chain comprises an amino acid sequence having at least 92% sequence identity to any one of SEQ ID NOs: 228-295 and 559-569. In some aspects described herein, the variable heavy chain comprises an amino acid sequence having at least 93% sequence identity to any one of SEQ ID NOs: 228-295 and 559-569. In some aspects described herein, the variable heavy chain comprises an amino acid sequence having at least 94% sequence identity to any one of SEQ ID NOs: 228-295 and 559-569. In some aspects described herein, the variable heavy chain comprises an amino acid sequence having at least 95% sequence identity to any one of SEQ ID NOs: 228-295 and 559-569. In some aspects described herein, the variable heavy chain comprises an amino acid sequence having at least 96% sequence identity to any one of SEQ ID NOs: 228-295 and 559-569. In some aspects described herein, the variable heavy chain comprises an amino acid sequence having at least 97% sequence identity to any one of SEQ ID NOs: 228-295 and 559-569. In some aspects described herein, the variable heavy chain comprises an amino acid sequence having at least 98% sequence identity to any one of SEQ ID NOs: 228-295 and 559-569. In some aspects described herein, the variable heavy chain comprises an amino acid sequence having at least 99% sequence identity to any one of SEQ ID NOs: 228-295 and 559-569. In some aspects described herein, the variable heavy chain comprises an amino acid sequence according to any one of SEQ ID NOs: 228-295 and 559-569.

In some aspects described herein, the antibody or antigen-binding fragment comprising a heavy chain variable region (VH) as described above, further comprises a light chain variable region (VL) having an amino acid sequence according to any of SEQ ID NOs: 296-369 and 570-583.

In some aspects described herein, an antibody or antigen-binding fragment thereof, is capable of specifically binding to a TCR Vβ region, comprising a light chain variable region (VL) having an amino acid sequence according to any of SEQ ID NOs: 296-369 and 570-583. In some aspects described herein, the variable light chain comprises an amino acid sequence having at least 85% sequence identity to any one of SEQ ID NOs: 296-369 and 570-583. In some aspects described herein, the variable light chain comprises an amino acid sequence having at least 86% sequence identity to any one of SEQ ID NOs: 296-369 and 570-583. In some aspects described herein, the variable light chain comprises an amino acid sequence having at least 87% sequence identity to any one of SEQ ID NOs: 296-369 and 570-583. In some aspects described herein, the variable light chain comprises an amino acid sequence having at least 88% sequence identity to any one of SEQ ID NOs: 296-369 and 570-583. In some aspects described herein, the variable light chain comprises an amino acid sequence having at least 89% sequence identity to any one of SEQ ID NOs: 296-369 and 570-583. In some aspects described herein, the variable light chain comprises an amino acid sequence having at least 90% sequence identity to any one of SEQ ID NOs: 296-369 and 570-583. In some aspects described herein, the variable light chain comprises an amino acid sequence having at least 91% sequence identity to any one of SEQ ID NOs: 296-369 and 570-583. In some aspects described herein, the variable light chain comprises an amino acid sequence having at least 92% sequence identity to any one of SEQ ID NOs: 296-369 and 570-583. In some aspects described herein, the variable light chain comprises an amino acid sequence having at least 93% sequence identity to any one of SEQ ID NOs: 296-369 and 570-583. In some aspects described herein, the variable light chain comprises an amino acid sequence having at least 94% sequence identity to any one of SEQ ID NOs: 296-369 and 570-583. In some aspects described herein, the variable light chain comprises an amino acid sequence having at least 95% sequence identity to any one of SEQ ID NOs: 296-369 and 570-583. In some aspects described herein, the variable light chain comprises an amino acid sequence having at least 96% sequence identity to any one of SEQ ID NOs: 296-369 and 570-583. In some aspects described herein, the variable light chain comprises an amino acid sequence having at least 97% sequence identity to any one of SEQ ID NOs: 296-369 and 570-583. In some aspects described herein, the variable light chain comprises an amino acid sequence having at least 98% sequence identity to any one of SEQ ID NOs: 296-369 and 570-583. In some aspects described herein, the variable light chain comprises an amino acid sequence having at least 99% sequence identity to any one of SEQ ID NOs: 296-369 and 570-583. In some aspects described herein, the variable light chain comprises an amino acid sequence according to any one of SEQ ID NOs: 296-369 and 570-583.

In some aspects described herein the antibody or antigen-binding fragment comprising a light chain variable region (VL) as described above, further comprises a heavy chain variable region (VH) having an amino acid sequence according to any of SEQ ID NOs: 228-295 and 559-569.

In some aspects described herein, the variable heavy chain comprises an amino acid sequence having any percent identity described above to any one of SEQ ID NOs: 228-295 and 559-569, and the variable light chain comprises an amino acid sequence having any percent identity described above to any one of SEQ ID NOs: 296-369 and 570-583. To illustrate, a non-limiting example pairing of variable heavy and light chains includes a variable heavy chain comprising an amino acid sequence having at least 88% sequence identity to any one of SEQ ID NOs: 228-295 and 559-569, and a variable light chain comprising an amino acid sequence having at least 93% sequence identity to any one of SEQ ID NOs: 296-369 and 570-583. In other aspects described herein, pairing of variable heavy and light chains includes a variable heavy chain comprising an amino acid sequence having any percent identity described above to any one of SEQ ID NOs: 228-295 and 559-569, and a variable light chain comprising an amino acid sequence according to any one of SEQ ID NOs: 296-369 and 570-583. In other aspects described herein, pairing of variable heavy and light chains includes a variable heavy chain comprising an amino acid sequence according to any one of SEQ ID NOs: 228-295 and 559-569, and a variable light chain comprising an amino acid sequence having any percent identity described above to any one of SEQ ID NOs: 296-369 and 570-583. To illustrate, a non-limiting example pairing of variable heavy and light chains includes a variable heavy chain comprising an amino acid sequence having at least 92% sequence identity to any one of SEQ ID NOs: 228-295 and 559-569, and a variable light chain comprising an amino acid sequence according to any one of SEQ ID NOs: 296-369 and 570-583. In yet other aspects described herein, the variable heavy chain comprises an amino acid sequence according to any one of SEQ ID NOs: 228-295 and 559-569, and the variable light chain comprises an amino acid sequence according to any one of SEQ ID NOs: 296-369 and 570-583.

In some aspects described herein, the antibody or antigen-binding fragment thereof is capable of specifically binding to TCR Vβ2 and, for example comprises (i) a variable heavy chain (VH), and/or a variable light chain (VL). In some aspects, the Vβ2 antibody or antigen binding fragment, for example, comprises (i) a variable heavy chain (VH) according to SEQ ID NO: 228, and/or (ii) a variable light chain (VL) according to SEQ ID NO: 296; SEQ ID NO: 297; SEQ ID NO: 298; SEQ ID NO: 299; SEQ ID NO: 300; SEQ ID NO: 301; SEQ ID NO: 302; or SEQ ID NO: 303. In some aspects, the Vβ2 antibody or antigen binding fragment, for example, comprises (i) a variable heavy chain (VH) according to SEQ ID NO: 229, and/or (ii) a variable light chain (VL) according to SEQ ID NO: 296; SEQ ID NO: 297; SEQ ID NO: 298; SEQ ID NO: 299; SEQ ID NO: 300; SEQ ID NO: 301; SEQ ID NO: 302; or SEQ ID NO: 303. In some aspects, the Vβ2 antibody or antigen binding fragment, for example, comprises (i) a variable heavy chain (VH) according to SEQ ID NO: 230, and/or (ii) a variable light chain (VL) according to SEQ ID NO: 296; SEQ ID NO: 297; SEQ ID NO: 298; SEQ ID NO: 299; SEQ ID NO: 300; SEQ ID NO: 301; SEQ ID NO: 302; or SEQ ID NO: 303. In some aspects, the Vβ2 antibody or antigen binding fragment, for example, comprises (i) a variable heavy chain (VH) according to SEQ ID NO: 231, and/or (ii) a variable light chain (VL) according to SEQ ID NO: 296; SEQ ID NO: 297; SEQ ID NO: 298; SEQ ID NO: 299; SEQ ID NO: 300; SEQ ID NO: 301; SEQ ID NO: 302; or SEQ ID NO: 303. In some aspects, the Vβ2 antibody or antigen binding fragment, for example, comprises (i) a variable heavy chain (VH) according to SEQ ID NO: 232, and/or (ii) a variable light chain (VL) according to SEQ ID NO: 296; SEQ ID NO: 297; SEQ ID NO: 298; SEQ ID NO: 299; SEQ ID NO: 300; SEQ ID NO: 301; SEQ ID NO: 302; or SEQ ID NO: 303. In some aspects, the Vβ2 antibody or antigen binding fragment, for example, comprises (i) a variable heavy chain (VH) according to SEQ ID NO: 233, and/or (ii) a variable light chain (VL) according to SEQ ID NO: 296; SEQ ID NO: 297; SEQ ID NO: 298; SEQ ID NO: 299; SEQ ID NO: 300; SEQ ID NO: 301; SEQ ID NO: 302; or SEQ ID NO: 303. In some aspects, the Vβ2 antibody or antigen binding fragment, for example, comprises (i) a variable heavy chain (VH) according to SEQ ID NO: 234, and/or (ii) a variable light chain (VL) according to SEQ ID NO: 296; SEQ ID NO: 297; SEQ ID NO: 298; SEQ ID NO: 299; SEQ ID NO: 300; SEQ ID NO: 301; SEQ ID NO: 302; or SEQ ID NO: 303. In some aspects, the Vβ2 antibody or antigen binding fragment, for example, comprises (i) a variable heavy chain (VH) according to SEQ ID NO: 235, and/or (ii) a variable light chain (VL) according to SEQ ID NO: 296; SEQ ID NO: 297; SEQ ID NO: 298; SEQ ID NO: 299; SEQ ID NO: 300; SEQ ID NO: 301; SEQ ID NO: 302; or SEQ ID NO: 303. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen binding fragment thereof is capable of specifically binding to Vβ7.2 and, for example comprises (i) a variable heavy chain (VH), and/or a variable light chain (VL). In some aspects, the Vβ7.2 antibody or antigen binding fragment, for example, comprises (i) a variable heavy chain (VH) according to SEQ ID NO: 236, and/or (ii) a variable light chain (VL) according to SEQ ID NO: 304; SEQ ID NO: 305; SEQ ID NO: 306; or SEQ ID NO: 307. In some aspects, the Vβ7.2 antibody or antigen binding fragment, for example, comprises (i) a variable heavy chain (VH) according to SEQ ID NO: 237, and/or (ii) a variable light chain (VL) according to SEQ ID NO: 304; SEQ ID NO: 305; SEQ ID NO: 306; or SEQ ID NO: 307. In some aspects, the Vβ7.2 antibody or antigen binding fragment, for example, comprises (i) a variable heavy chain (VH) according to SEQ ID NO: 238, and/or (ii) a variable light chain (VL) according to SEQ ID NO: 304; SEQ ID NO: 305; SEQ ID NO: 306; or SEQ ID NO: 307. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen binding fragment thereof is capable of specifically binding to Vβ4, and, for example comprises (i) a variable heavy chain (VH), and/or a variable light chain (VL). In some aspects, the Vβ4 antibody or antigen binding fragment, for example, comprises (i) a variable heavy chain (VH) according to SEQ ID NO: 239, and/or (ii) a variable light chain (VL) according to SEQ ID NO: 308; or SEQ ID NO: 309. In some aspects, the Vβ4 antibody or antigen binding fragment, for example, comprises (i) a variable heavy chain (VH) according to SEQ ID NO: 240, and/or (ii) a variable light chain (VL) according to SEQ ID NO: 308; or SEQ ID NO: 309. In some aspects, the Vβ4 antibody or antigen binding fragment, for example, comprises (i) a variable heavy chain (VH) according to SEQ ID NO: 241, and/or (ii) a variable light chain (VL) according to SEQ ID NO: 308; or SEQ ID NO: 309. In some aspects, the Vβ4 antibody or antigen binding fragment, for example, comprises (i) a variable heavy chain (VH) according to SEQ ID NO: 242, and/or (ii) a variable light chain (VL) according to SEQ ID NO: 308; or SEQ ID NO: 309. In some aspects, the Vβ4 antibody or antigen binding fragment, for example, comprises (i) a variable heavy chain (VH) according to SEQ ID NO: 243, and/or (ii) a variable light chain (VL) according to SEQ ID NO: 308; or SEQ ID NO: 309. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen binding fragment thereof is capable of specifically binding to Vβ13.2 and, for example comprises (i) a variable heavy chain (VH), and/or a variable light chain (VL), In some aspects described herein, the antibody or antigen binding fragment thereof is capable of specifically binding to Vβ13.2 and, for example, comprises (i) a variable heavy chain (VH) according to SEQ ID NO: 244, and/or (ii) a variable light chain (VL) according to SEQ ID NO: 310; SEQ ID NO: 311; SEQ ID NO: 312; SEQ ID NO: 313; SEQ ID NO: 314; SEQ ID NO: 315; SEQ ID NO: 316; or SEQ ID NO: 317. In some aspects, the Vβ13.2 antibody or antigen binding fragment, for example, comprises (i) a variable heavy chain (VH) according to SEQ ID NO: 245, and/or (ii) a variable light chain (VL) according to SEQ ID NO: 310; SEQ ID NO: 311; SEQ ID NO: 312; SEQ ID NO: 313; SEQ ID NO: 314; SEQ ID NO: 315; SEQ ID NO: 316; or SEQ ID NO: 317. In some aspects, the Vβ13.2 antibody or antigen binding fragment, for example, comprises (i) a variable heavy chain (VH) according to SEQ ID NO: 246, and/or (ii) a variable light chain (VL) according to SEQ ID NO: 310; SEQ ID NO: 311; SEQ ID NO: 312; SEQ ID NO: 313; SEQ ID NO: 314; SEQ ID NO: 315; SEQ ID NO: 316; or SEQ ID NO: 317. In some aspects, the Vβ13.2 antibody or antigen binding fragment, for example, comprises (i) a variable heavy chain (VH) according to SEQ ID NO: 247, and/or (ii) a variable light chain (VL) according to SEQ ID NO: 310; SEQ ID NO: 311; SEQ ID NO: 312; SEQ ID NO: 313; SEQ ID NO: 314; SEQ ID NO: 315; SEQ ID NO: 316; or SEQ ID NO: 317. In some aspects, the Vβ13.2 antibody or antigen binding fragment, for example, comprises (i) a variable heavy chain (VH) according to SEQ ID NO: 248, and/or (ii) a variable light chain (VL) according to SEQ ID NO: 310; SEQ ID NO: 311; SEQ ID NO: 312; SEQ ID NO: 313; SEQ ID NO: 314; SEQ ID NO: 315; SEQ ID NO: 316; or SEQ ID NO: 317. In some aspects, the Vβ13.2 antibody or antigen binding fragment, for example, comprises (i) a variable heavy chain (VH) according to SEQ ID NO: 249, and/or (ii) a variable light chain (VL) according to SEQ ID NO: 310; SEQ ID NO: 311; SEQ ID NO: 312; SEQ ID NO: 313; SEQ ID NO: 314; SEQ ID NO: 315; SEQ ID NO: 316; or SEQ ID NO: 317. In some aspects, the Vβ13.2 antibody or antigen binding fragment, for example, comprises (i) a variable heavy chain (VH) according to SEQ ID NO: 250, and/or (ii) a variable light chain (VL) according to SEQ ID NO: 310; SEQ ID NO: 311; SEQ ID NO: 312; SEQ ID NO: 313; SEQ ID NO: 314; SEQ ID NO: 315; SEQ ID NO: 316; or SEQ ID NO: 317. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen binding fragment thereof is capable of specifically binding to V814 and, for example comprises (i) a variable heavy chain (VH), and/or a variable light chain (VL). In some aspects, the V814 antibody or antigen binding fragment, for example, comprises (i) a variable heavy chain (VH) according to SEQ ID NO: 251, and/or (ii) a variable light chain (VL) according to SEQ ID NO: 318; or SEQ ID NO: 319. SEQ ID NO: 320; SEQ ID NO: 321; SEQ ID NO: 322; or SEQ ID NO: 323. In some aspects, the V814 antibody or antigen binding fragment, for example, comprises (i) a variable heavy chain (VH) according to SEQ ID NO: 252, and/or (ii) a variable light chain (VL) according to SEQ ID NO: 318; or SEQ ID NO: 319. SEQ ID NO: 320; SEQ ID NO: 321; SEQ ID NO: 322; or SEQ ID NO: 323. In some aspects, the V814 antibody or antigen binding fragment, for example, comprises (i) a variable heavy chain (VH) according to SEQ ID NO: 253, and/or (ii) a variable light chain (VL) according to SEQ ID NO: 318; or SEQ ID NO: 319. SEQ ID NO: 320; SEQ ID NO: 321; SEQ ID NO: 322; or SEQ ID NO: 323. In some aspects, the V814 antibody or antigen binding fragment, for example, comprises (i) a variable heavy chain (VH) according to SEQ ID NO: 254, and/or (ii) a variable light chain (VL) according to SEQ ID NO: 318; or SEQ ID NO: 319. SEQ ID NO: 320; SEQ ID NO: 321; SEQ ID NO: 322; or SEQ ID NO: 323. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen binding fragment thereof is capable of specifically binding to Vβ22 and, for example comprises (i) a variable heavy chain (VH), and/or a variable light chain (VL). In some aspects, the Vβ22 antibody or antigen binding fragment, for example, comprises (i) a variable heavy chain (VH) according to SEQ ID NO: 255, and/or (ii) a variable light chain (VL) according to SEQ ID NO: 324; SEQ ID NO: 325; SEQ ID NO: 326; or SEQ ID NO: 327. In some aspects, the Vβ22 antibody or antigen binding fragment, for example, comprises (i) a variable heavy chain (VH) according to SEQ ID NO: 256, and/or (ii) a variable light chain (VL) according to SEQ ID NO: 324; SEQ ID NO: 325; SEQ ID NO: 326; or SEQ ID NO: 327. In some aspects, the Vβ22 antibody or antigen binding fragment, for example, comprises (i) a variable heavy chain (VH) according to SEQ ID NO: 257, and/or (ii) a variable light chain (VL) according to SEQ ID NO: 324; SEQ ID NO: 325; SEQ ID NO: 326; or SEQ ID NO: 327. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen binding fragment thereof is capable of specifically binding to Vβ11 and, for example comprises (i) a variable heavy chain (VH), and/or a variable light chain (VL). In some aspects, the Vβ11 antibody or antigen binding fragment, for example, comprises (i) a variable heavy chain (VH) according to SEQ ID NO: 258, and/or (ii) a variable light chain (VL) according to SEQ ID NO: 328; SEQ ID NO: 329; SEQ ID NO: 330; or SEQ ID NO: 331. In some aspects, the Vβ11 antibody or antigen binding fragment, for example, comprises (i) a variable heavy chain (VH) according to SEQ ID NO: 259, and/or (ii) a variable light chain (VL) according to SEQ ID NO: 328; SEQ ID NO: 329; SEQ ID NO: 330; or SEQ ID NO: 331, In some aspects, the Vβ11 antibody or antigen binding fragment, for example, comprises (i) a variable heavy chain (VH) according to SEQ ID NO: 260, and/or (ii) a variable light chain (VL) according to SEQ ID NO: 328; SEQ ID NO: 329; SEQ ID NO: 330; or SEQ ID NO: 331. In some aspects, the Vβ11 antibody or antigen binding fragment, for example, comprises (i) a variable heavy chain (VH) according to SEQ ID NO: 261, and/or (ii) a variable light chain (VL) according to SEQ ID NO: 332; SEQ ID NO: 333; SEQ ID NO: 334; SEQ ID NO: 335; or SEQ ID NO: 336. In some aspects, the Vβ11 antibody or antigen binding fragment, for example, comprises (i) a variable heavy chain (VH) according to SEQ ID NO: 262, and/or (ii) a variable light chain (VL) according to SEQ ID NO: 332; SEQ ID NO: 333; SEQ ID NO: 334; SEQ ID NO: 335; or SEQ ID NO: 336. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen binding fragment thereof is capable of specifically binding to Vβ12 and, for example comprises (i) a variable heavy chain (VH), and/or a variable light chain (VL). In some aspects, the Vβ12 antibody or antigen binding fragment, for example, comprises (i) a variable heavy chain (VH) according to SEQ ID NO: 263, and/or (ii) a variable light chain (VL) according to SEQ ID NO: 337; SEQ ID NO: 338; SEQ ID NO: 339; SEQ ID NO: 340; or SEQ ID NO: 341. In some aspects, the Vβ12 antibody or antigen binding fragment, for example, comprises (i) a variable heavy chain (VH) according to SEQ ID NO: 264, and/or (ii) a variable light chain (VL) according to SEQ ID NO: 337; SEQ ID NO: 338; SEQ ID NO: 339; SEQ ID NO: 340; or SEQ ID NO: 341. In some aspects, the Vβ12 antibody or antigen binding fragment, for example, comprises (i) a variable heavy chain (VH) according to SEQ ID NO: 265, and/or (ii) a variable light chain (VL) according to SEQ ID NO: 337; SEQ ID NO: 338; SEQ ID NO: 339; SEQ ID NO: 340; or SEQ ID NO: 341. In some aspects, the Vβ12 antibody or antigen binding fragment, for example, comprises (i) a variable heavy chain (VH) according to SEQ ID NO: 266, and/or (ii) a variable light chain (VL) according to SEQ ID NO: 337; SEQ ID NO: 338; SEQ ID NO: 339; SEQ ID NO: 340; or SEQ ID NO: 341. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen binding fragment thereof is capable of specifically binding to Vβ8 and, for example comprises (i) a variable heavy chain (VH), and/or a variable light chain (VL). In some aspects, the Vβ8 antibody or antigen binding fragment, for example, comprises (i) a variable heavy chain (VH) according to SEQ ID NO: 267, and/or (ii) a variable light chain (VL) according to SEQ ID NO: 342; SEQ ID NO: 343; or SEQ ID NO: 344. In some aspects, the Vβ8 antibody or antigen binding fragment, for example, comprises (i) a variable heavy chain (VH) according to SEQ ID NO: 268, and/or (ii) a variable light chain (VL) according to SEQ ID NO: 342; SEQ ID NO: 343; or SEQ ID NO: 344. In some aspects, the Vβ8 antibody or antigen binding fragment, for example, comprises (i) a variable heavy chain (VH) according to SEQ ID NO: 269, and/or (ii) a variable light chain (VL) according to SEQ ID NO: 342; SEQ ID NO: 343; or SEQ ID NO: 344. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen binding fragment thereof is capable of specifically binding to Vβ13.1 and, for example comprises (i) a variable heavy chain (VH), and/or a variable light chain (VL). In some aspects, the Vβ13.1 antibody or antigen binding fragment, for example, comprises (i) a variable heavy chain (VH) according to SEQ ID NO: 270, and/or (ii) a variable light chain (VL) according to SEQ ID NO: 345; SEQ ID NO: 346; or SEQ ID NO: 347. In some aspects, the Vβ13.1 antibody or antigen binding fragment, for example, comprises (i) a variable heavy chain (VH) according to SEQ ID NO: 271, and/or (ii) a variable light chain (VL) according to SEQ ID NO: 345; SEQ ID NO: 346; or SEQ ID NO: 347. In some aspects, the Vβ13.1 antibody or antigen binding fragment, for example, comprises (i) a variable heavy chain (VH) according to SEQ ID NO: 272, and/or (ii) a variable light chain (VL) according to SEQ ID NO: 345; SEQ ID NO: 346; or SEQ ID NO: 347. In some aspects, the Vβ13.1 antibody or antigen binding fragment, for example, comprises (i) a variable heavy chain (VH) according to SEQ ID NO: 273, and/or (ii) a variable light chain (VL) according to SEQ ID NO: 345; SEQ ID NO: 346; or SEQ ID NO: 347. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen binding fragment thereof is capable of specifically binding to Vβ5.1 and, for example comprises (i) a variable heavy chain (VH), and/or a variable light chain (VL). In some aspects, the Vβ5.1 antibody or antigen binding fragment, for example, comprises (i) a variable heavy chain (VH) according to SEQ ID NO: 274, and/or (ii) a variable light chain (VL) according to SEQ ID NO: 348; SEQ ID NO: 349; or SEQ ID NO: 350. In some aspects, the Vβ5.1 antibody or antigen binding fragment, for example, comprises (i) a variable heavy chain (VH) according to SEQ ID NO: 275, and/or (ii) a variable light chain (VL) according to SEQ ID NO: 348; SEQ ID NO: 349; or SEQ ID NO: 350. In some aspects, the Vβ5.1 antibody or antigen binding fragment, for example, comprises (i) a variable heavy chain (VH) according to SEQ ID NO: 276, and/or (ii) a variable light chain (VL) according to SEQ ID NO: 348; SEQ ID NO: 349; or SEQ ID NO: 350. In some aspects, the Vβ5.1 antibody or antigen binding fragment, for example, comprises (i) a variable heavy chain (VH) according to SEQ ID NO: 277, and/or (ii) a variable light chain (VL) according to SEQ ID NO: 348; SEQ ID NO: 349; or SEQ ID NO: 350. In some aspects, the Vβ5.1 antibody or antigen binding fragment, for example, comprises (i) a variable heavy chain (VH) according to SEQ ID NO: 278, and/or (ii) a variable light chain (VL) according to SEQ ID NO: 348; SEQ ID NO: 349; or SEQ ID NO: 350. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen binding fragment thereof is capable of specifically binding to Vβ9 and, for example comprises (i) a variable heavy chain (VH), and/or a variable light chain (VL). In some aspects, the Vβ9 antibody or antigen binding fragment, for example, comprises (i) a variable heavy chain (VH) according to SEQ ID NO: 279, and/or (ii) a variable light chain (VL) according to SEQ ID NO: 351; SEQ ID NO: 352; SEQ ID NO: 353; SEQ ID NO: 354. In some aspects, the Vβ9 antibody or antigen binding fragment, for example, comprises (i) a variable heavy chain (VH) according to SEQ ID NO: 280, and/or (ii) a variable light chain (VL) according to SEQ ID NO: 351; SEQ ID NO: 352; SEQ ID NO: 353; or SEQ ID NO: 354, In some aspects, the Vβ9 antibody or antigen binding fragment, for example, comprises (i) a variable heavy chain (VH) according to SEQ ID NO: 281, and/or (ii) a variable light chain (VL) according to SEQ ID NO: 351; SEQ ID NO: 352; SEQ ID NO: 353; or SEQ ID NO: 354. In some aspects, the Vβ9 antibody or antigen binding fragment, for example, comprises (i) a variable heavy chain (VH) according to SEQ ID NO: 282, and/or (ii) a variable light chain (VL) according to SEQ ID NO: 351; SEQ ID NO: 352; SEQ ID NO: 353; or SEQ ID NO: 354. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen binding fragment thereof is capable of specifically binding to Vβ17 and, for example comprises (i) a variable heavy chain (VH), and/or a variable light chain (VL). In some aspects, the Vβ17 antibody or antigen binding fragment, for example, comprises (i) a variable heavy chain (VH) according to SEQ ID NO: 283, and/or (ii) a variable light chain (VL) according to SEQ ID NO: 355; SEQ ID NO: 356; or SEQ ID NO: 357. In some aspects, the Vβ17 antibody or antigen binding fragment, for example, comprises (i) a variable heavy chain (VH) according to SEQ ID NO: 284, and/or (ii) a variable light chain (VL) according to SEQ ID NO: 355; SEQ ID NO: 356; or SEQ ID NO: 357. In some aspects, the Vβ17 antibody or antigen binding fragment, for example, comprises (i) a variable heavy chain (VH) according to SEQ ID NO: 285, and/or (ii) a variable light chain (VL) according to SEQ ID NO: 355; SEQ ID NO: 356; or SEQ ID NO: 357. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen binding fragment thereof is capable of specifically binding to Vβ3 and, for example comprises (i) a variable heavy chain (VH), and/or a variable light chain (VL). In some aspects, the Vβ3 antibody or antigen binding fragment, for example, comprises (i) a variable heavy chain (VH) according to SEQ ID NO: 286, and/or (ii) a variable light chain (VL) according to SEQ ID NO: 358; SEQ ID NO: 359; or SEQ ID NO: 360. In some aspects, the Vβ3 antibody or antigen binding fragment, for example, comprises (i) a variable heavy chain (VH) according to SEQ ID NO: 287, and/or (ii) a variable light chain (VL) according to SEQ ID NO: 358; SEQ ID NO: 359; or SEQ ID NO: 360. In some aspects, the Vβ3 antibody or antigen binding fragment, for example, comprises (i) a variable heavy chain (VH) according to SEQ ID NO: 289, and/or (ii) a variable light chain (VL) according to SEQ ID NO: 358; SEQ ID NO: 359; or SEQ ID NO: 360. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen binding fragment thereof is capable of specifically binding to Vβ7.1 and, for example comprises (i) a variable heavy chain (VH), and/or a variable light chain (VL). In some aspects, the Vβ7.1 antibody or antigen binding fragment, for example, comprises (i) a variable heavy chain (VH) according to SEQ ID NO: 290, and/or (ii) a variable light chain (VL) according to SEQ ID NO: 361; SEQ ID NO: 362; SEQ ID NO: 363; SEQ ID NO: 364; SEQ ID NO: 365; SEQ ID NO: 366; or SEQ ID NO: 367. In some aspects, the Vβ7.1 antibody or antigen binding fragment, for example, comprises (i) a variable heavy chain (VH) according to SEQ ID NO: 291, and/or (ii) a variable light chain (VL) according to SEQ ID NO: 361; SEQ ID NO: 362; SEQ ID NO: 363; SEQ ID NO: 364; SEQ ID NO: 365; SEQ ID NO: 366; or SEQ ID NO: 367. In some aspects, the Vβ7.1 antibody or antigen binding fragment, for example, comprises (i) a variable heavy chain (VH) according to SEQ ID NO: 292, and/or (ii) a variable light chain (VL) according to SEQ ID NO: 361; SEQ ID NO: 362; SEQ ID NO: 363; SEQ ID NO: 364; SEQ ID NO: 365; SEQ ID NO: 366; or SEQ ID NO: 367. In some aspects, the Vβ7.1 antibody or antigen binding fragment, for example, comprises (i) a variable heavy chain (VH) according to SEQ ID NO: 293, and/or (ii) a variable light chain (VL) according to SEQ ID NO: 361; SEQ ID NO: 362; SEQ ID NO: 363; SEQ ID NO: 364; SEQ ID NO: 365; SEQ ID NO: 366; or SEQ ID NO: 367. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen binding fragment thereof is capable of specifically binding to Vβ5.3 and, for example comprises (i) a variable heavy chain (VH), and/or a variable light chain (VL). In some aspects, the Vβ5.3 antibody or antigen binding fragment, for example, comprises (i) a variable heavy chain (VH) according to SEQ ID NO: 294, and/or (ii) a variable light chain (VL) according to SEQ ID NO: 368 or SEQ ID NO: 369. In some aspects, the Vβ5.3 antibody or antigen binding fragment, for example, comprises (i) a variable heavy chain (VH) according to SEQ ID NO: 295, and/or (ii) a variable light chain (VL) according to SEQ ID NO: 368 or SEQ ID NO: 369. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen binding fragment thereof is capable of specifically binding to Vβ20 and, for example comprises (i) a variable heavy chain (VH), and/or a variable light chain (VL). In some aspects, the Vβ20 antibody or antigen binding fragment, for example, comprises (i) a variable heavy chain (VH) according to SEQ ID NO: 559, and/or (ii) a variable light chain (VL) according to SEQ ID NO: 570; SEQ ID NO: 571; or SEQ ID NO: 572. In some aspects, the Vβ20 antibody or antigen binding fragment, for example, comprises (i) a variable heavy chain (VH) according to SEQ ID NO: 560, and/or (ii) a variable light chain (VL) according to SEQ ID NO: 570; SEQ ID NO: 571; or SEQ ID NO: 572. In some aspects, the Vβ20 antibody or antigen binding fragment, for example, comprises (i) a variable heavy chain (VH) according to SEQ ID NO: 561, and/or (ii) a variable light chain (VL) according to SEQ ID NO: 570; SEQ ID NO: 571; or SEQ ID NO: 572. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen binding fragment thereof is capable of specifically binding to Vβ18 and, for example comprises (i) a variable heavy chain (VH), and/or a variable light chain (VL). In some aspects, the Vβ18 antibody or antigen binding fragment, for example, comprises (i) a variable heavy chain (VH) according to SEQ ID NO: 562, and/or (ii) a variable light chain (VL) according to SEQ ID NO: 573; SEQ ID NO: 574; SEQ ID NO: 575. In some aspects, the Vβ18 antibody or antigen binding fragment, for example, comprises (i) a variable heavy chain (VH) according to SEQ ID NO: 563, and/or (ii) a variable light chain (VL) according to SEQ ID NO: 573; SEQ ID NO: 574; SEQ ID NO: 575. In some aspects, the Vβ18 antibody or antigen binding fragment, for example, comprises (i) a variable heavy chain (VH) according to SEQ ID NO: 564, and/or (ii) a variable light chain (VL) according to SEQ ID NO: 573; SEQ ID NO: 574; SEQ ID NO: 575. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen binding fragment thereof is capable of specifically binding to Vβ13.6 and, for example comprises (i) a variable heavy chain (VH), and/or a variable light chain (VL). In some aspects, the Vβ13.6 antibody or antigen binding fragment, for example, comprises (i) a variable heavy chain (VH) according to SEQ ID NO: 565, and/or (ii) a variable light chain (VL) according to SEQ ID NO: 576; SEQ ID NO: 577. In some aspects, the Vβ13.6 antibody or antigen binding fragment, for example, comprises (i) a variable heavy chain (VH) according to SEQ ID NO: 566, and/or (ii) a variable light chain (VL) according to SEQ ID NO: 576; SEQ ID NO: 577. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen binding fragment thereof is capable of specifically binding to Vβ16 and, for example comprises (i) a variable heavy chain (VH), and/or a variable light chain (VL). In some aspects, the Vβ16 antibody or antigen binding fragment, for example, comprises (i) a variable heavy chain (VH) according to SEQ ID NO: 567, and/or (ii) a variable light chain (VL) according to SEQ ID NO: 578; SEQ ID NO: 579; SEQ ID NO: 580; SEQ ID NO: 581; SEQ ID NO: 582; or SEQ ID NO: 583. In some aspects, the Vβ16 antibody or antigen binding fragment, for example, comprises (i) a variable heavy chain (VH) according to SEQ ID NO: 568, and/or (ii) a variable light chain (VL) according to SEQ ID NO: 578; SEQ ID NO: 579; SEQ ID NO: 580; SEQ ID NO: 581; SEQ ID NO: 582; or SEQ ID NO: 583. In some aspects, the Vβ16 antibody or antigen binding fragment, for example, comprises (i) a variable heavy chain (VH) according to SEQ ID NO: 569, and/or (ii) a variable light chain (VL) according to SEQ ID NO: 578; SEQ ID NO: 579; SEQ ID NO: 580; SEQ ID NO: 581; SEQ ID NO: 582; or SEQ ID NO: 583. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects described herein, the antibody or antigen-binding fragment thereof, is human, humanized, or chimeric. In a preferred aspect, the antibody or antigen-binding fragment thereof, is humanized.

In some aspects described herein, the antibody or antigen-binding fragment thereof, is an IgG antibody. In some aspects described herein, the IgG antibody, is an IgG1, IgG2, IgG3, or an IgG4 antibody. In some aspects described herein, the IgG antibody, is an IgG1 antibody or an IgG4 antibody. In some aspects described herein, the IgG antibody is an IgG1 antibody.

In some aspects described herein, the antibody, is an antigen-binding fragment of an antibody. In some aspects described herein, the antigen-binding fragment, is selected from the group consisting of Fab, F(ab′)2, Fv, scFv, scFv-Fc, dsFv and a single domain molecule. In some aspects described herein, the antigen-binding fragment, is an scFv. In some aspects described herein, the antigen-binding fragment, is a Fab. In some aspects described herein, the antigen-binding fragment, is an intrabody. In some aspects described herein, the antigen-binding fragment, is devoid of an Fc region.

In some aspects described herein, the antibody or antigen-binding fragment, comprises a VH and a VL on the same polypeptide chain. In some aspects described herein, the antibody or antigen-binding fragment, comprises a VH and a VL on separate polypeptide chains.

In some aspects described herein, the antibody or antigen-binding fragment VH and VL are connected by a linker.

In some aspects described herein, the TCR Vβ region is selected from the group consisting of Vβ1, Vβ2, Vβ3, Vβ4, Vβ5.1, Vβ5.3, Vβ6, Vβ7.1, Vβ7.2, Vβ8, Vβ9, Vβ10, Vβ11, Vβ12, Vβ13.1, Vβ13.2, Vβ14, Vβ15, Vβ16, Vβ17, Vβ18, Vβ19, Vβ20, Vβ21, Vβ22, Vβ23, Vβ24, Vβ25, Vβ26, Vβ27, Vβ28, Vβ29, Vβ30, VβA, VβB, and VβC. It will be understood that a combination of variable heavy and light chains described herein that specifically bind to the same TCR Vβ region are capable of providing increased in vitro and/or in vivo activity compared to a combination of variable heavy and light chains described herein that specifically bind to different TCR Vβ regions.

In some aspects described herein, the antibody or antigen-binding fragment TCR Vβ region is selected from the group consisting of Vβ1, Vβ2, Vβ3, Vβ4, Vβ5.1, Vβ5.3, Vβ7.1, Vβ7.2, Vβ8, Vβ9, Vβ11, Vβ 12, Vβ 13.1, Vβ 13.2, Vβ 14, Vβ 17, and Vβ22.

In some aspects described herein, the antibody or antigen-binding fragment TCR Vβ region is Vβ2.

In some aspects, for the expression of antibodies or antigen-binding fragments thereof, vectors encoding both the heavy and light chains, individually, can be co-expressed in the host cell for expression of the entire immunoglobulin, as detailed below.

In some aspects, a host cell contains a vector comprising a polynucleotide encoding both the heavy chain and light chain of an antibody disclosed herein or a domain thereof.

In some aspects, a host cell contains two different vectors, a first vector comprising a polynucleotide encoding a heavy chain or a heavy chain variable region of an antibody or antigen-binding fragment thereof disclosed herein, and a second vector comprising a polynucleotide encoding a light chain or a light chain variable region of an antibody disclosed herein or a domain thereof. In some aspects, a first host cell comprises a first vector comprising a polynucleotide encoding a heavy chain or a heavy chain variable region of an antibody or antigen-binding fragment thereof disclosed herein, and a second host cell comprises a second vector comprising a polynucleotide encoding a light chain or a light chain variable region of an antibody or antigen-binding fragment thereof disclosed herein. In some aspects, a heavy chain/heavy chain variable region expressed by a first cell associated with a light chain/light chain variable region of a second cell to form an anti-TCR Vβ antibody or antigen-binding fragment thereof disclosed herein. In some aspects, provided herein is a population of host cells comprising such first host cell and such second host cell.

In some aspects, provided herein is a population of vectors comprising a first vector comprising a polynucleotide encoding a light chain/light chain variable region of an anti-TCR Vβ antibody or antigen-binding fragment thereof disclosed herein, and a second vector comprising a polynucleotide encoding a heavy chain/heavy chain variable region of an anti-TCR Vβ antibody or antigen-binding fragment thereof disclosed herein. Alternatively, a single vector can be used which encodes, and is capable of expressing, both heavy and light chain polypeptides.

A variety of host-expression vector systems can be utilized to express antibodies and antigen-binding fragments thereof disclosed herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antibody or antigen-binding fragment thereof disclosed herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7030, HsS78Bst, HeLa, and NIH 3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some aspects, cells for expressing antibodies and antigen-binding fragments thereof disclosed herein are CHO cells, for example CHO cells from the CHO GS System™ (Lonza). In some aspects, cells for expressing antibodies disclosed herein are human cells, e.g., human cell lines. In some aspects, a mammalian expression vector is pOptiVEC™ or pcDNA3.3. In some aspects, bacterial cells such as Escherichia coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells in conjunction with a vector such as the major intermediate early gene promoter element from human cytomegalovirus is an effective expression system for antibodies (Foecking M K & Hofstetter H (1986) Gene 45: 101-105; and Cockett M I et al., (1990) Biotechnology 8: 662-667). In some aspects, antibodies or antigen-binding fragments thereof disclosed herein are produced by CHO cells or NS0 cells.

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells that possess the cellular machinery for proper processing of the primary transcript, glycosylation, and phosphorylation of the gene product can be used. Such mammalian host cells include but are not limited to CHO, VERO, BHK, HeLa, MDCK, HEK 293, NIH 3T3, W138, BT483, Hs578T, HTB2, BT20 and T47D, NS0 (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells. In some aspects, anti-TCR Vβ antibodies disclosed herein are produced in mammalian cells, such as CHO cells, e.g., CHO-K1 cells. In some aspects, anti-TCR Vβ antibodies disclosed herein are produced in mammalian cells, such as HEK-293 cells.

Once an antibody or antigen-binding fragment thereof disclosed herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies or antigen-binding fragments thereof disclosed herein can be fused to heterologous polypeptide sequences disclosed herein or otherwise known in the art to facilitate purification.

In some aspects, an antibody or antigen-binding fragment thereof for example, disclosed herein is isolated or purified. Generally, an isolated antibody or antigen-binding fragment thereof is one that is substantially free of other antibodies or antigen-binding fragments thereof with different antigenic specificities than the isolated antibody or antigen-binding fragment thereof. For example, in some aspects, a preparation of an antibody or antigen-binding fragment thereof disclosed herein is substantially free of cellular material and/or chemical precursors.

In some aspects the antibody or antigen binding fragment is modified to enhance antibody-dependent cell-mediated cytotoxicity (ADCC).

In some aspects, the ADCC activity of the antibody composition is increased or decreased by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 1-fold, about 2-fold, about 3-fold, or about 4-fold, or increased or decreased by about 5% to about 400%.

In some aspects, the antibody or antigen binding fragment has an increased antibody-dependent cell mediated cytotoxicity activity achieved through afucosylation.

In some aspects, the antibody or antigen binding fragment has an increased antibody-dependent cell mediated cytotoxicity activity achieved through glycosylation.

### III. Methods of Treatment

In some aspects described herein are methods for treating cancer in a subject in need thereof, the method comprising: administering to the subject a therapeutically effective amount of a T-cell composition comprising a CAR, the CAR comprises (i) an antigen binding molecule that specifically binds to a TCR Vβ region, (ii) an extracellular domain, (iii) a transmembrane domain, (iv) a costimulatory domain and (v) an activating domain, the antigen binding molecule is a single chain variable fragment (scFv) comprising: a variable heavy chain according to any one of SEQ ID NOs: 45-131 and 534-544, or a variable light chain according to any one of SEQ ID NOs: 132-227 and 545-558.

In some aspects described herein are methods for treating T-cell mediated diseases in a subject in need thereof, the method comprising: administering to the subject a therapeutically effective amount of a T-cell composition comprising a CAR, the CAR comprises (i) an antigen binding molecule that specifically binds to a TCR Vβ region, (ii) an extracellular domain, (iii) a transmembrane domain, (iv) a costimulatory domain and (v) an activating domain, the antigen binding molecule is a single chain variable fragment (scFv) comprising: a variable heavy chain according to any one of SEQ ID NOs: 45-131 and 534-544, or a variable light chain according to any one of SEQ ID NOs: 132-227 and 545-558.

In some aspects of the methods described herein, the chimeric antigen receptor comprises (i) the variable heavy chain (VH) which comprises CDR1, CDR2 and CDR3 according to SEQ ID NO:10, SEQ ID NO:11, and SEQ ID NO:12, respectively, or (ii) the variable light chain (VL) comprises CDR1, CDR2 and CDR3 according to SEQ ID NO:13, SEQ ID NO:14, SEQ ID NO:15, respectively; or (i) the variable heavy chain (VH) comprises CDR1, CDR2 and CDR3 according to SEQ ID NO:370, SEQ ID NO:371, and SEQ ID NO:372, respectively, and/or (ii) the variable light chain (VL) comprises CDR1, CDR2 and CDR3 according to SEQ ID NO:418, SEQ ID NO:419, SEQ ID NO:420, respectively; or (i) the variable heavy chain (VH) comprises CDR1, CDR2 and CDR3 according to SEQ ID NO:373, SEQ ID NO:374, and SEQ ID NO:375 respectively, and/or (ii) the variable light chain (VL) comprises CDR1, CDR2 and CDR3 according to SEQ ID NO:421, SEQ ID NO:422, SEQ ID NO:423, respectively; or (i) the variable heavy chain (VH) comprises CDR1, CDR2 and CDR3 according to SEQ ID NO:376, SEQ ID NO:377, and SEQ ID NO:378, respectively, and/or (ii) the variable light chain (VL) comprises CDR1, CDR2 and CDR3 according to SEQ ID NO:424, SEQ ID NO:425, SEQ ID NO:426, respectively; or the variable heavy chain (VH) comprises CDR1, CDR2 and CDR3 according to SEQ ID NO:379, SEQ ID NO:380, and SEQ ID NO:381 respectively, and/or (ii) the variable light chain (VL) comprises CDR1, CDR2 and CDR3 according to SEQ ID NO:427, SEQ ID NO:428, SEQ ID NO:429, respectively; or (i) the variable heavy chain (VH) comprises CDR1, CDR2 and CDR3 according to SEQ ID NO:382, SEQ ID NO:383, and SEQ ID NO:384, respectively, and/or (ii) the variable light chain (VL) comprises CDR1, CDR2 and CDR3 according to SEQ ID NO:430, SEQ ID NO:431, SEQ ID NO:432, respectively; or (i) the variable heavy chain (VH) comprises CDR1, CDR2 and CDR3 according to SEQ ID NO:385, SEQ ID NO:386, and SEQ ID NO:387, respectively, and/or (ii) the variable light chain (VL) comprises CDR1, CDR2 and CDR3 according to SEQ ID NO:433, SEQ ID NO:434, SEQ ID NO:435, respectively; or (i) the variable heavy chain (VH) comprises CDR1, CDR2 and CDR3 according to SEQ ID NO:388, SEQ ID NO:389, and SEQ ID NO:390, respectively, and/or (ii) the variable light chain (VL) comprises CDR1, CDR2 and CDR3 according to SEQ ID NO:436, SEQ ID NO:437, SEQ ID NO:438, respectively; or (i) the variable heavy chain (VH) comprises CDR1, CDR2 and CDR3 according to SEQ ID NO:391, SEQ ID NO:392, and SEQ ID NO:393 respectively, and/or (ii) the variable light chain (VL) comprises CDR1, CDR2 and CDR3 according to SEQ ID NO:439, SEQ ID NO:440, SEQ ID NO:441, respectively; or (i) the variable heavy chain (VH) comprises CDR1, CDR2 and CDR3 according to SEQ ID NO:394, SEQ ID NO:395, and SEQ ID NO:396, respectively, and/or (ii) the variable light chain (VL) comprises CDR1, CDR2 and CDR3 according to SEQ ID NO:442, SEQ ID NO:443, SEQ ID NO:444, respectively; or (i) the variable heavy chain (VH) comprises CDR1, CDR2 and CDR3 according to SEQ ID NO:397, SEQ ID NO:398, and SEQ ID NO:399, respectively, and/or (ii) the variable light chain (VL) comprises CDR1, CDR2 and CDR3 according to SEQ ID NO:445, SEQ ID NO:446, SEQ ID NO:447, respectively; or (i) the variable heavy chain (VH) comprises CDR1, CDR2 and CDR3 according to SEQ ID NO:400, SEQ ID NO:401, and SEQ ID NO:402, respectively, and/or (ii) the variable light chain (VL) comprises CDR1, CDR2 and CDR3 according to SEQ ID NO:448, SEQ ID NO:449, SEQ ID NO:450, respectively; or (i) the variable heavy chain (VH) comprises CDR1, CDR2 and CDR3 according to SEQ ID NO:403, SEQ ID NO:404, and SEQ ID NO:405 respectively, and/or (ii) the variable light chain (VL) comprises CDR1, CDR2 and CDR3 according to SEQ ID NO:451, SEQ ID NO:452, SEQ ID NO:453, respectively; or (i) the variable heavy chain (VH) comprises CDR1, CDR2 and CDR3 according to SEQ ID NO:406, SEQ ID NO:407, and SEQ ID NO:408, respectively, and/or (ii) the variable light chain (VL) comprises CDR1, CDR2 and CDR3 according to SEQ ID NO:454, SEQ ID NO:455, SEQ ID NO:456, respectively; or (i) the variable heavy chain (VH) comprises CDR1, CDR2 and CDR3 according to SEQ ID NO:409, SEQ ID NO:410, and SEQ ID NO:411, respectively, and/or (ii) the variable light chain (VL) comprises CDR1, CDR2 and CDR3 according to SEQ ID NO:457, SEQ ID NO:458, SEQ ID NO:459, respectively; or (i) the variable heavy chain (VH) comprises CDR1, CDR2 and CDR3 according to SEQ ID NO:412, SEQ ID NO:413, and SEQ ID NO:414, respectively, and/or (ii) the variable light chain (VL) comprises CDR1, CDR2 and CDR3 according to SEQ ID NO:460, SEQ ID NO:461, SEQ ID NO:462, respectively; or (i) the variable heavy chain (VH) comprises CDR1, CDR2 and CDR3 according to SEQ ID NO:415, SEQ ID NO:416, and SEQ ID NO:417, respectively, and/or (ii) the variable light chain (VL) comprises CDR1, CDR2 and CDR3 according to SEQ ID NO:463, SEQ ID NO:464, SEQ ID NO:465, respectively; or (i) the variable heavy chain (VH) comprises CDR1, CDR2 and CDR3 according to SEQ ID NO:584, SEQ ID NO:585, and SEQ ID NO:586, respectively, and/or (ii) the variable light chain (VL) comprises CDR1, CDR2 and CDR3 according to SEQ ID NO:596, SEQ ID NO:597, SEQ ID NO:598, respectively; or (i) the variable heavy chain (VH) comprises CDR1, CDR2 and CDR3 according to SEQ ID NO:587, SEQ ID NO:588, and SEQ ID NO:589, respectively, and/or (ii) the variable light chain (VL) comprises CDR1, CDR2 and CDR3 according to SEQ ID NO:599, SEQ ID NO:601, SEQ ID NO:601, respectively; or (i) the variable heavy chain (VH) comprises CDR1, CDR2 and CDR3 according to SEQ ID NO:590, SEQ ID NO:591, and SEQ ID NO:592, respectively, and/or (ii) the variable light chain (VL) comprises CDR1, CDR2 and CDR3 according to SEQ ID NO:602, SEQ ID NO:603, SEQ ID NO:604, respectively; or (i) the variable heavy chain (VH) comprises CDR1, CDR2 and CDR3 according to SEQ ID NO:593, SEQ ID NO:594, and SEQ ID NO:595, respectively, and/or (ii) the variable light chain (VL) comprises CDR1, CDR2 and CDR3 according to SEQ ID NO:605, SEQ ID NO:606, SEQ ID NO:607, respectively, or SEQ ID NO: 608, SEQ ID NO: 609, SEQ ID NO: 610, respectively.

In some aspects of the methods described herein, the variable heavy chain comprises an amino acid sequence having at least 85% sequence identity to any one of SEQ ID NOs: 45-131 and 534-544. In some aspects described herein, the variable heavy chain comprises an amino acid sequence having at least 86% sequence identity to any one of SEQ ID NOs: 45-131 and 534-544. In some aspects described herein, the variable heavy chain comprises an amino acid sequence having at least 87% sequence identity to any one of SEQ ID NOs: 45-131 and 534-544. In some aspects described herein, the variable heavy chain comprises an amino acid sequence having at least 88% sequence identity to any one of SEQ ID NOs: 45-131 and 534-544. In some aspects described herein, the variable heavy chain comprises an amino acid sequence having at least 89% sequence identity to any one of SEQ ID NOs: 45-131 and 534-544. In some aspects described herein, the variable heavy chain comprises an amino acid sequence having at least 90% sequence identity to any one of SEQ ID NOs: 45-131 and 534-544. In some aspects described herein, the variable heavy chain comprises an amino acid sequence having at least 91% sequence identity to any one of SEQ ID NOs: 45-131 and 534-544. In some aspects described herein, the variable heavy chain comprises an amino acid sequence having at least 92% sequence identity to any one of SEQ ID NOs: 45-131 and 534-544. In some aspects described herein, the variable heavy chain comprises an amino acid sequence having at least 93% sequence identity to any one of SEQ ID NOs: 45-131 and 534-544. In some aspects described herein, the variable heavy chain comprises an amino acid sequence having at least 94% sequence identity to any one of SEQ ID NOs: 45-131 and 534-544. In some aspects described herein, the variable heavy chain comprises an amino acid sequence having at least 95% sequence identity to any one of SEQ ID NOs: 45-131 and 534-544. In some aspects described herein, the variable heavy chain comprises an amino acid sequence having at least 96% sequence identity to any one of SEQ ID NOs: 45-131 and 534-544. In some aspects described herein, the variable heavy chain comprises an amino acid sequence having at least 97% sequence identity to any one of SEQ ID NOs: 45-131 and 534-544. In some aspects described herein, the variable heavy chain comprises an amino acid sequence having at least 98% sequence identity to any one of SEQ ID NOs: 45-131 and 534-544. In some aspects described herein, the variable heavy chain comprises an amino acid sequence having at least 99% sequence identity to any one of SEQ ID NOs: 45-131 and 534-544. In some aspects described herein, the variable heavy chain comprises an amino acid sequence according to any one of SEQ ID NOs: 45-131 and 534-544.

In some aspects of the methods described herein, the variable light chain comprises an amino acid sequence having at least 85% sequence identity to any one of SEQ ID NOs: 132-227 and 545-558. In some aspects described herein, the variable light chain comprises an amino acid sequence having at least 86% sequence identity to any one of SEQ ID NOs: 132-227 and 545-558. In some aspects described herein, the variable light chain comprises an amino acid sequence having at least 87% sequence identity to any one of SEQ ID NOs: 132-227 and 545-558. In some aspects described herein, the variable light chain comprises an amino acid sequence having at least 88% sequence identity to any one of SEQ ID NOs: 132-227 and 545-558. In some aspects described herein, the variable light chain comprises an amino acid sequence having at least 89% sequence identity to any one of SEQ ID NOs: 132-227 and 545-558. In some aspects described herein, the variable light chain comprises an amino acid sequence having at least 90% sequence identity to any one of SEQ ID NOs: 132-227 and 545-558. In some aspects described herein, the variable light chain comprises an amino acid sequence having at least 91% sequence identity to any one of SEQ ID NOs: 132-227 and 545-558. In some aspects described herein, the variable light chain comprises an amino acid sequence having at least 92% sequence identity to any one of SEQ ID NOs: 132-227 and 545-558. In some aspects described herein, the variable light chain comprises an amino acid sequence having at least 93% sequence identity to any one of SEQ ID NOs: 132-227 and 545-558. In some aspects described herein, the variable light chain comprises an amino acid sequence having at least 94% sequence identity to any one of SEQ ID NOs: 132-227 and 545-558. In some aspects described herein, the variable light chain comprises an amino acid sequence having at least 95% sequence identity to any one of SEQ ID NOs: 132-227 and 545-558. In some aspects described herein, the variable light chain comprises an amino acid sequence having at least 96% sequence identity to any one of SEQ ID NOs: 132-227 and 545-558. In some aspects described herein, the variable light chain comprises an amino acid sequence having at least 97% sequence identity to any one of SEQ ID NOs: 132-227 and 545-558. In some aspects described herein, the variable light chain comprises an amino acid sequence having at least 98% sequence identity to any one of SEQ ID NOs: 132-227 and 545-558. In some aspects described herein, the variable light chain comprises an amino acid sequence having at least 99% sequence identity to any one of SEQ ID NOs: 132-227 and 545-558. In some aspects described herein, the variable light chain comprises an amino acid sequence according to any one of SEQ ID NOs: 132-227 and 545-558.

In some aspects of the methods described herein, the variable heavy chain comprises an amino acid sequence having any percent identity described above to any one of SEQ ID NOs: 45-131 and 534-544, and the variable light chain comprises an amino acid sequence having any percent identity described above to any one of SEQ ID NOs: 132-227 and 545-558. To illustrate, a non-limiting example pairing of variable heavy and light chains includes a variable heavy chain comprising an amino acid sequence having at least 88% sequence identity to any one of SEQ ID NOs: 45-131 and 534-544, and a variable light chain comprising an amino acid sequence having at least 93% sequence identity to any one of SEQ ID NOs: 132-227 and 545-558. In other aspects of the methods decribed herein, pairing of variable heavy and light chains includes a variable heavy chain comprising an amino acid sequence having any percent identity described above to any one of SEQ ID NOs: 45-131 and 534-544, and a variable light chain comprising an amino acid sequence according to any one of SEQ ID NOs: 132-227 and 545-558. In other aspects described herein, pairing of variable heavy and light chains includes a variable heavy chain comprising an amino acid sequence according to any one of SEQ ID NOs: 45-131 and 534-544, and a variable light chain comprising an amino acid sequence having any percent identity described above to any one of SEQ ID NOs: 132-227 and 545-558. To illustrate, a non-limiting example pairing of variable heavy and light chains includes a variable heavy chain comprising an amino acid sequence having at least 92% sequence identity to any one of SEQ ID NOs: 45-131 and 534-544, and a variable light chain comprising an amino acid sequence according to any one of SEQ ID NOs: 132-227 and 545-558. In yet other aspects described herein, the variable heavy chain comprises an amino acid sequence according to any one of SEQ ID NOs: 45-131 and 534-544, and the variable light chain comprises an amino acid sequence according to any one of SEQ ID NOs: 132-227 and 545-558.

In some aspects of the methods described herein, the CAR is capable of specifically binding to Vβ2 and, for example, comprises (i) a variable heavy chain (VH), and/or a variable light chain (VL). In some aspects, the Vβ2 CAR, for example comprises (i) a variable heavy chain (VH) according to SEQ ID NO: 45, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 46, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 47, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 48, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 49, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 50, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 51, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 52, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 53, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 54, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 55, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 56, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 57, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 58, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 59, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 60, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 61, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 62, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 63, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 64, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 65, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 66, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 67, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 68, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 69, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 70, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 71, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the Vβ2 CAR, for example comprises (i) a variable heavy chain (VH) according to SEQ ID NO: 72, and/or (ii) a variable light chain (VL) according to SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; or SEQ ID NO: 159. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the Vβ7.2 CAR, for example comprises (i) a variable heavy chain (VH) according to SEQ ID NO: 73, and/or (ii) a variable light chain (VL) according to SEQ ID NO: 160; SEQ ID NO: 161; SEQ ID NO: 162; or SEQ ID NO: 163. In some aspects, the Vβ7.2 CAR, for example comprises (i) a variable heavy chain (VH) according to SEQ ID NO: 74, and/or (ii) a variable light chain (VL) according to SEQ ID NO: 160; SEQ ID NO: 161; SEQ ID NO: 162; or SEQ ID NO: 163. In some aspects, the Vβ7.2 CAR, for example comprises (i) a variable heavy chain (VH) according to SEQ ID NO: 75, and/or (ii) a variable light chain (VL) according to SEQ ID NO: 160; SEQ ID NO: 161; SEQ ID NO: 162; or SEQ ID NO: 163. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the CAR is capable of specifically binding to Vβ4 and, for example, comprises (i) a variable heavy chain (VH), and/or a variable light chain (VL). In some aspects, the Vβ4 CAR, for example comprises (i) a variable heavy chain (VH) according to SEQ ID NO: 76, and/or (ii) a variable light chain (VL) according to SEQ ID NO: 164; SEQ ID NO: 165; SEQ ID NO: 166; or SEQ ID NO: 167. In some aspects, the Vβ4 CAR, for example comprises (i) a variable heavy chain (VH) according to SEQ ID NO: 77, and/or (ii) a variable light chain (VL) according to SEQ ID NO: 164; SEQ ID NO: 165; SEQ ID NO: 166; or SEQ ID NO: 167. In some aspects, the Vβ4 CAR, for example comprises (i) a variable heavy chain (VH) according to SEQ ID NO: 78, and/or (ii) a variable light chain (VL) according to SEQ ID NO: 164; SEQ ID NO: 165; SEQ ID NO: 166; or SEQ ID NO: 167. In some aspects, the Vβ4 CAR, for example comprises (i) a variable heavy chain (VH) according to SEQ ID NO: 79, and/or (ii) a variable light chain (VL) according to SEQ ID NO: 164; SEQ ID NO: 165; SEQ ID NO: 166; or SEQ ID NO: 167. In some aspects, the Vβ4 CAR, for example comprises (i) a variable heavy chain (VH) according to SEQ ID NO: 80, and/or (ii) a variable light chain (VL) according to SEQ ID NO: 164; SEQ ID NO: 165; SEQ ID NO: 166; or SEQ ID NO: 167. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the CAR is capable of specifically binding to Vβ13.2 and, for example, comprises (i) a variable heavy chain (VH), and/or a variable light chain (VL). In some aspects, the Vβ13.2 CAR, for example comprises (i) a variable heavy chain (VH) according to SEQ ID NO: 81, and/or (ii) a variable light chain (VL) according to SEQ ID NO: 168; SEQ ID NO: 169; SEQ ID NO: 170; SEQ ID NO: 171; SEQ ID NO: 172; SEQ ID NO: 173; SEQ ID NO: 174; or SEQ ID NO: 175. In some aspects, the Vβ13.2 CAR, for example comprises (i) a variable heavy chain (VH) according to SEQ ID NO: 82, and/or (ii) a variable light chain (VL) according to SEQ ID NO: 168; SEQ ID NO: 169; SEQ ID NO: 170; SEQ ID NO: 171; SEQ ID NO: 172; SEQ ID NO: 173; SEQ ID NO: 174; or SEQ ID NO: 175. In some aspects, the Vβ13.2 CAR, for example comprises (i) a variable heavy chain (VH) according to SEQ ID NO: 83, and/or (ii) a variable light chain (VL) according to SEQ ID NO: 168; SEQ ID NO: 169; SEQ ID NO: 170; SEQ ID NO: 171; SEQ ID NO: 172; SEQ ID NO: 173; SEQ ID NO: 174; or SEQ ID NO: 175. In some aspects, the Vβ13.2 CAR, for example comprises (i) a variable heavy chain (VH) according to SEQ ID NO: 84, and/or (ii) a variable light chain (VL) according to SEQ ID NO: 168; SEQ ID NO: 169; SEQ ID NO: 170; SEQ ID NO: 171; SEQ ID NO: 172; SEQ ID NO: 173; SEQ ID NO: 174; or SEQ ID NO: 175. In some aspects, the Vβ13.2 CAR, for example comprises (i) a variable heavy chain (VH) according to SEQ ID NO: 85, and/or (ii) a variable light chain (VL) according to SEQ ID NO: 168; SEQ ID NO: 169; SEQ ID NO: 170; SEQ ID NO: 171; SEQ ID NO: 172; SEQ ID NO: 173; SEQ ID NO: 174; or SEQ ID NO: 175. In some aspects, the Vβ13.2 CAR, for example comprises (i) a variable heavy chain (VH) according to SEQ ID NO: 86, and/or (ii) a variable light chain (VL) according to SEQ ID NO: 168; SEQ ID NO: 169; SEQ ID NO: 170; SEQ ID NO: 171; SEQ ID NO: 172; SEQ ID NO: 173; SEQ ID NO: 174; or SEQ ID NO: 175. In some aspects, the Vβ13.2 CAR, for example comprises (i) a variable heavy chain (VH) according to SEQ ID NO: 87, and/or (ii) a variable light chain (VL) according to SEQ ID NO: 168; SEQ ID NO: 169; SEQ ID NO: 170; SEQ ID NO: 171; SEQ ID NO: 172; SEQ ID NO: 173; SEQ ID NO: 174; or SEQ ID NO: 175. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the CAR is capable of specifically binding to V814 and, for example, comprises (i) a variable heavy chain (VH), and/or a variable light chain (VL). In some aspects, the V814 CAR, for example comprises (i) a variable heavy chain (VH) according to SEQ ID NO: 88, and/or (ii) a variable light chain (VL) according to SEQ ID NO: 176; SEQ ID NO: 177; SEQ ID NO: 178; SEQ ID NO: 179; SEQ ID NO: 180; or SEQ ID NO: 181. In some aspects, the Vβ14 CAR, for example comprises (i) a variable heavy chain (VH) according to SEQ ID NO: 89, and/or (ii) a variable light chain (VL) according to SEQ ID NO: 176; SEQ ID NO: 177; SEQ ID NO: 178; SEQ ID NO: 179; SEQ ID NO: 180; or SEQ ID NO: 181. In some aspects, the Vβ14 CAR, for example comprises (i) a variable heavy chain (VH) according to SEQ ID NO: 90, and/or (ii) a variable light chain (VL) according to SEQ ID NO: 176; SEQ ID NO: 177; SEQ ID NO: 178; SEQ ID NO: 179; SEQ ID NO: 180; or SEQ ID NO: 181. In some aspects, the Vβ14 CAR, for example comprises (i) a variable heavy chain (VH) according to SEQ ID NO: 91, and/or (ii) a variable light chain (VL) according to SEQ ID NO: 176; SEQ ID NO: 177; SEQ ID NO: 178; SEQ ID NO: 179; SEQ ID NO: 180; or SEQ ID NO: 181. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the CAR is capable of specifically binding to Vβ22 and, for example, comprises (i) a variable heavy chain (VH), and/or a variable light chain (VL). In some aspects, the Vβ22 CAR, for example comprises (i) a variable heavy chain (VH) according to SEQ ID NO: 92, and/or (ii) a variable light chain (VL) according to SEQ ID NO: 182; SEQ ID NO: 183; SEQ ID NO: 184; or SEQ ID NO: 185. In some aspects, the Vβ22 CAR, for example comprises (i) a variable heavy chain (VH) according to SEQ ID NO: 93, and/or (ii) a variable light chain (VL) according to SEQ ID NO: 182; SEQ ID NO: 183; SEQ ID NO: 184; or SEQ ID NO: 185. In some aspects, the Vβ22 CAR, for example comprises (i) a variable heavy chain (VH) according to SEQ ID NO: 94, and/or (ii) a variable light chain (VL) according to SEQ ID NO: 182; SEQ ID NO: 183; SEQ ID NO: 184; or SEQ ID NO: 185. In some aspects, the Vβ22 CAR, for example comprises (i) a variable heavy chain (VH) according to SEQ ID NO: 95, and/or (ii) a variable light chain (VL) according to SEQ ID NO: 182; SEQ ID NO: 183; SEQ ID NO: 184; or SEQ ID NO: 185. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the CAR is capable of specifically binding to Vβ11 and, for example, comprises (i) a variable heavy chain (VH), and/or a variable light chain (VL). In some aspects, the Vβ11 CAR, for example comprises (i) a variable heavy chain (VH) according to SEQ ID NO: 96, and/or (ii) a variable light chain (VL) according to SEQ ID NO: 186; SEQ ID NO: 187; SEQ ID NO: 188; or SEQ ID NO: 189. In some aspects, the Vβ11 CAR, for example comprises (i) a variable heavy chain (VH) according to SEQ ID NO: 97, and/or (ii) a variable light chain (VL) according to SEQ ID NO: 186; SEQ ID NO: 187; SEQ ID NO: 188; or SEQ ID NO: 189. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the CAR is capable of specifically binding to Vβ1 and, for example, comprises (i) a variable heavy chain (VH), and/or a variable light chain (VL). In some aspects, the Vβ1 CAR, for example comprises (i) a variable heavy chain (VH) according to SEQ ID NO: 98, and/or (ii) a variable light chain (VL) according to SEQ ID NO: 190; SEQ ID NO: 191; SEQ ID NO: 192; SEQ ID NO: 193; or SEQ ID NO: 194. In some aspects, the Vβ1 CAR, for example comprises (i) a variable heavy chain (VH) according to SEQ ID NO: 99, and/or (ii) a variable light chain (VL) according to SEQ ID NO: 190; SEQ ID NO: 191; SEQ ID NO: 192; SEQ ID NO: 193; or SEQ ID NO: 194. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the CAR is capable of specifically binding to Vβ12 and, for example, comprises (i) a variable heavy chain (VH), and/or a variable light chain (VL). In some aspects, the Vβ12 CAR, for example comprises (i) a variable heavy chain (VH) according to SEQ ID NO: 100, and/or (ii) a variable light chain (VL) according to SEQ ID NO: 195; SEQ ID NO: 196; SEQ ID NO: 197; SEQ ID NO: 198; or SEQ ID NO: 199. In some aspects, the Vβ12 CAR, for example comprises (i) a variable heavy chain (VH) according to SEQ ID NO: 101, and/or (ii) a variable light chain (VL) according to SEQ ID NO: 195; SEQ ID NO: 196; SEQ ID NO: 197; SEQ ID NO: 198; or SEQ ID NO: 199. In some aspects, the Vβ12 CAR, for example comprises (i) a variable heavy chain (VH) according to SEQ ID NO: 102, and/or (ii) a variable light chain (VL) according to SEQ ID NO: 195; SEQ ID NO: 196; SEQ ID NO: 197; SEQ ID NO: 198; or SEQ ID NO: 199. In some aspects, the Vβ12 CAR, for example comprises (i) a variable heavy chain (VH) according to SEQ ID NO: 103, and/or (ii) a variable light chain (VL) according to SEQ ID NO: 195; SEQ ID NO: 196; SEQ ID NO: 197; SEQ ID NO: 198; or SEQ ID NO: 199. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the CAR is capable of specifically binding to Vβ8 and, for example, comprises (i) a variable heavy chain (VH), and/or a variable light chain (VL). In some aspects, the Vβ8 CAR, for example comprises (i) a variable heavy chain (VH) according to SEQ ID NO: 104, and/or (ii) a variable light chain (VL) according to SEQ ID NO: 200; SEQ ID NO: 201; or SEQ ID NO: 202. In some aspects, the Vβ8 CAR, for example comprises (i) a variable heavy chain (VH) according to SEQ ID NO: 105, and/or (ii) a variable light chain (VL) according to SEQ ID NO: 200; SEQ ID NO: 201; or SEQ ID NO: 202. In some aspects, the Vβ8 CAR, for example comprises (i) a variable heavy chain (VH) according to SEQ ID NO: 106, and/or (ii) a variable light chain (VL) according to SEQ ID NO: 200; SEQ ID NO: 201; or SEQ ID NO: 202. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the CAR is capable of specifically binding to Vβ13.1 and, for example, comprises (i) a variable heavy chain (VH), and/or a variable light chain (VL). In some aspects, the Vβ13.1 CAR, for example comprises (i) a variable heavy chain (VH) according to SEQ ID NO: 107, and/or (ii) a variable light chain (VL) according to SEQ ID NO: 203; SEQ ID NO: 204; or SEQ ID NO: 205. In some aspects, the Vβ13.1 CAR, for example comprises (i) a variable heavy chain (VH) according to SEQ ID NO: 108, and/or (ii) a variable light chain (VL) according to SEQ ID NO: 203; SEQ ID NO: 204; or SEQ ID NO: 205. In some aspects, the Vβ13.1 CAR, for example comprises (i) a variable heavy chain (VH) according to SEQ ID NO: 109, and/or (ii) a variable light chain (VL) according to SEQ ID NO: 203; SEQ ID NO: 204; or SEQ ID NO: 205. In some aspects, the Vβ13.1 CAR, for example comprises (i) a variable heavy chain (VH) according to SEQ ID NO: 110, and/or (ii) a variable light chain (VL) according to SEQ ID NO: 203; SEQ ID NO: 204; or SEQ ID NO: 205. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the CAR is capable of specifically binding to Vβ5.1 and, for example, comprises (i) a variable heavy chain (VH), and/or a variable light chain (VL). In some aspects, the Vβ5.1 CAR, for example comprises (i) a variable heavy chain (VH) according to SEQ ID NO: 111, and/or (ii) a variable light chain (VL) according to SEQ ID NO: 206; SEQ ID NO: 207; or SEQ ID NO: 208. In some aspects, the Vβ5.1 CAR, for example comprises (i) a variable heavy chain (VH) according to SEQ ID NO: 112, and/or (ii) a variable light chain (VL) according to SEQ ID NO: 206; SEQ ID NO: 207; or SEQ ID NO: 208. In some aspects, the Vβ5.1 CAR, for example comprises (i) a variable heavy chain (VH) according to SEQ ID NO: 113, and/or (ii) a variable light chain (VL) according to SEQ ID NO: 206; SEQ ID NO: 207; or SEQ ID NO: 208. In some aspects, the Vβ5.1 CAR, for example comprises (i) a variable heavy chain (VH) according to SEQ ID NO: 114, and/or (ii) a variable light chain (VL) according to SEQ ID NO: 206; SEQ ID NO: 207; or SEQ ID NO: 208. In some aspects, the Vβ5.1 CAR, for example comprises (i) a variable heavy chain (VH) according to SEQ ID NO: 115, and/or (ii) a variable light chain (VL) according to SEQ ID NO: 206; SEQ ID NO: 207; or SEQ ID NO: 208. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the CAR is capable of specifically binding to Vβ9 and, for example, comprises (i) a variable heavy chain (VH), and/or a variable light chain (VL). In some aspects, the Vβ9 CAR, for example comprises (i) a variable heavy chain (VH) according to SEQ ID NO: 116, and/or (ii) a variable light chain (VL) according to SEQ ID NO: 209; SEQ ID NO: 210; SEQ ID NO: 211; or SEQ ID NO: 212. In some aspects, the Vβ9 CAR, for example comprises (i) a variable heavy chain (VH) according to SEQ ID NO: 117, and/or (ii) a variable light chain (VL) according to SEQ ID NO: 209; SEQ ID NO: 210; SEQ ID NO: 211; or SEQ ID NO: 212. In some aspects, the Vβ9 CAR, for example comprises (i) a variable heavy chain (VH) according to SEQ ID NO: 118, and/or (ii) a variable light chain (VL) according to SEQ ID NO: 209; SEQ ID NO: 210; SEQ ID NO: 211; or SEQ ID NO: 212. In some aspects, the Vβ9 CAR, for example comprises (i) a variable heavy chain (VH) according to SEQ ID NO: 119, and/or (ii) a variable light chain (VL) according to SEQ ID NO: 209; SEQ ID NO: 210; SEQ ID NO: 211; or SEQ ID NO: 212. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the CAR is capable of specifically binding to Vβ17 and, for example, comprises (i) a variable heavy chain (VH), and/or a variable light chain (VL). In some aspects, the Vβ17 CAR, for example comprises (i) a variable heavy chain (VH) according to SEQ ID NO: 120, and/or (ii) a variable light chain (VL) according to SEQ ID NO: 213; SEQ ID NO: 214; or SEQ ID NO: 215. In some aspects, the Vβ17 CAR, for example comprises (i) a variable heavy chain (VH) according to SEQ ID NO: 121, and/or (ii) a variable light chain (VL) according to SEQ ID NO: 213; SEQ ID NO: 214; or SEQ ID NO: 215. In some aspects, the Vβ17 CAR, for example comprises (i) a variable heavy chain (VH) according to SEQ ID NO: 122, and/or (ii) a variable light chain (VL) according to SEQ ID NO: 213; SEQ ID NO: 214; or SEQ ID NO: 215. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the CAR is capable of specifically binding to Vβ3 and, for example, comprises (i) a variable heavy chain (VH), and/or a variable light chain (VL). In some aspects, the Vβ3 CAR, for example comprises (i) a variable heavy chain (VH) according to SEQ ID NO: 123, and/or (ii) a variable light chain (VL) according to SEQ ID NO: 216; SEQ ID NO: 217; or SEQ ID NO: 218. In some aspects, the Vβ3 CAR, for example comprises (i) a variable heavy chain (VH) according to SEQ ID NO: 124, and/or (ii) a variable light chain (VL) according to SEQ ID NO: 216; SEQ ID NO: 217; or SEQ ID NO: 218. In some aspects, the Vβ3 CAR, for example comprises (i) a variable heavy chain (VH) according to SEQ ID NO: 125, and/or (ii) a variable light chain (VL) according to SEQ ID NO: 216; SEQ ID NO: 217; or SEQ ID NO: 218. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the CAR is capable of specifically binding to Vβ7.1 and, for example, comprises (i) a variable heavy chain (VH), and/or a variable light chain (VL). In some aspects, the Vβ7.1 CAR, for example comprises (i) a variable heavy chain (VH) according to SEQ ID NO: 126, and/or (ii) a variable light chain (VL) according to SEQ ID NO: 219; SEQ ID NO: 220; SEQ ID NO: 221; SEQ ID NO: 222; SEQ ID NO: 223; or SEQ ID NO: 224. In some aspects, the Vβ7.1 CAR, for example comprises (i) a variable heavy chain (VH) according to SEQ ID NO: 127, and/or (ii) a variable light chain (VL) according to SEQ ID NO: 219; SEQ ID NO: 220; SEQ ID NO: 221; SEQ ID NO: 222; SEQ ID NO: 223; or SEQ ID NO: 224. In some aspects, the Vβ7.1 CAR, for example comprises (i) a variable heavy chain (VH) according to SEQ ID NO: 128, and/or (ii) a variable light chain (VL) according to SEQ ID NO: 219; SEQ ID NO: 220; SEQ ID NO: 221; SEQ ID NO: 222; SEQ ID NO: 223; or SEQ ID NO: 224. In some aspects, the V87.1 CAR, for example comprises (i) a variable heavy chain (VH) according to SEQ ID NO: 129, and/or (ii) a variable light chain (VL) according to SEQ ID NO: 219; SEQ ID NO: 220; SEQ ID NO: 221; SEQ ID NO: 222; SEQ ID NO: 223; or SEQ ID NO: 224. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the CAR is capable of specifically binding to Vβ5.3 and, for example, comprises (i) a variable heavy chain (VH), and/or a variable light chain (VL). In some aspects, the Vβ5.3 CAR, for example comprises (i) a variable heavy chain (VH) according to SEQ ID NO: 130, and/or (ii) a variable light chain (VL) according to SEQ ID NO: 225; SEQ ID NO: 226; or SEQ ID NO: 227, In some aspects, the Vβ5.3 CAR, for example comprises (i) a variable heavy chain (VH) according to SEQ ID NO: 131, and/or (ii) a variable light chain (VL) according to SEQ ID NO: 225; SEQ ID NO: 226; or SEQ ID NO: 227. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the CAR is capable of specifically binding to Vβ20 and, for example, comprises (i) a variable heavy chain (VH), and/or a variable light chain (VL). In some aspects, the Vβ20 CAR, for example comprises (i) a variable heavy chain (VH) according to SEQ ID NO: 534, and/or (ii) a variable light chain (VL) according to SEQ ID NO: 545; SEQ ID NO: 546; or SEQ ID NO: 547. In some aspects, the Vβ20 CAR, for example comprises (i) a variable heavy chain (VH) according to SEQ ID NO: 535, and/or (ii) a variable light chain (VL) according to SEQ ID NO: 545; SEQ ID NO: 546; or SEQ ID NO: 547. In some aspects, the Vβ20 CAR, for example comprises (i) a variable heavy chain (VH) according to SEQ ID NO: 536, and/or (ii) a variable light chain (VL) according to SEQ ID NO: 545; SEQ ID NO: 546; or SEQ ID NO: 547. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the CAR is capable of specifically binding to Vβ18 and, for example, comprises (i) a variable heavy chain (VH), and/or a variable light chain (VL). In some aspects, the Vβ18 CAR, for example comprises (i) a variable heavy chain (VH) according to SEQ ID NO: 537, and/or (ii) a variable light chain (VL) according to SEQ ID NO: 548; SEQ ID NO: 549; or SEQ ID NO: 550. In some aspects, the Vβ18 CAR, for example comprises (i) a variable heavy chain (VH) according to SEQ ID NO: 538, and/or (ii) a variable light chain (VL) according to SEQ ID NO: 548; SEQ ID NO: 549; or SEQ ID NO: 550. In some aspects, the Vβ18 CAR, for example comprises (i) a variable heavy chain (VH) according to SEQ ID NO: 539, and/or (ii) a variable light chain (VL) according to SEQ ID NO: 548; SEQ ID NO: 549; or SEQ ID NO: 550. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the CAR is capable of specifically binding to Vβ13.6 and, for example, comprises (i) a variable heavy chain (VH), and/or a variable light chain (VL). In some aspects, the Vβ13.6 CAR, for example comprises (i) a variable heavy chain (VH) according to SEQ ID NO: 540, and/or (ii) a variable light chain (VL) according to SEQ ID NO: 551; or SEQ ID NO: 552. In some aspects, the Vβ13.6 CAR, for example comprises (i) a variable heavy chain (VH) according to SEQ ID NO: 541, and/or (ii) a variable light chain (VL) according to SEQ ID NO: 551; or SEQ ID NO: 552. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the CAR is capable of specifically binding to Vβ16 and, for example, comprises (i) a variable heavy chain (VH), and/or a variable light chain (VL). In some aspects, the Vβ16 CAR, for example comprises (i) a variable heavy chain (VH) according to SEQ ID NO: 542, and/or (ii) a variable light chain (VL) according to SEQ ID NO: 553; SEQ ID NO: 554; SEQ ID NO: 555; SEQ ID NO: 556; SEQ ID NO: 557; or SEQ ID NO: 558. In some aspects, the Vβ16 CAR, for example comprises (i) a variable heavy chain (VH) according to SEQ ID NO: 543, and/or (ii) a variable light chain (VL) according to SEQ ID NO: 553; SEQ ID NO: 554; SEQ ID NO: 555; SEQ ID NO: 556; SEQ ID NO: 557; or SEQ ID NO: 558. In some aspects, the Vβ16 CAR, for example comprises (i) a variable heavy chain (VH) according to SEQ ID NO: 544, and/or (ii) a variable light chain (VL) according to SEQ ID NO: 553; SEQ ID NO: 554; SEQ ID NO: 555; SEQ ID NO: 556; SEQ ID NO: 557; or SEQ ID NO: 558. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, a nucleic acid molecule encoding the VH of the chimeric antigen receptor is disclosed. In aspects described herein, a nucleic acid molecule encodes a polypeptide comprising the VH of the chimeric antigen receptor of any of SEQ ID NO: 45-131 and 534-544. In some aspects described herein, a nucleic acid molecule encoding the VL of the chimeric antigen receptor is disclosed. In aspects described herein, a nucleic acid molecule encodes a polypeptide comprising the VL of the chimeric antigen receptor of any of SEQ ID NO:132-227 and 545-558. In some aspects described herein, a nucleic acid molecule encoding the chimeric antigen receptor is disclosed. In aspects described herein, a nucleic acid molecule encoding a polypeptide comprising the chimeric antigen receptor of any of SEQ ID NO:16-44.

In some aspects described herein are methods for treating cancer in a subject in need thereof, the method comprising: administering to the subject a therapeutically effective amount of a composition comprising an antibody or antigen-binding fragment thereof capable of specifically binding to a TCR Vβ region, comprising a (i) heavy chain variable region (VH) having an amino acid sequence according to any of SEQ ID NOs: 228-295 and 559-569, or (ii) a light chain variable region (VL) having an amino acid sequence according to any of SEQ ID NOs: 296-369 and 570-583. In some aspects described herein are methods for treating cancer in a subject in need thereof, the method comprising: administering to the subject a therapeutically effective amount of a composition comprising an antibody or antigen-binding fragment thereof capable of specifically binding to a TCR Vβ region, comprising a (i) heavy chain variable region (VH) having an amino acid sequence according to any of SEQ ID NOs: 228-295 and 559-569, and (ii) a light chain variable region (VL) having an amino acid sequence according to any of SEQ ID NOs: 296-369 and 570-583.

In some aspects described herein are methods for treating T-cell mediated diseases in a subject in need thereof, the method comprising for example; administering to the subject a therapeutically effective amount of a composition comprising an antibody or antigen-binding fragment thereof capable of specifically binding to a TCR Vβ region, comprising a (i) heavy chain variable region (VH) having an amino acid sequence according to any of SEQ ID NOs: 228-295 and 559-569, or (ii) a light chain variable region (VL) having an amino acid sequence according to any of SEQ ID NOs: 228-295 and 559-569. In some aspects described herein are methods for treating T-cell mediated diseases in a subject in need thereof, the method comprising for example; administering to the subject a therapeutically effective amount of a composition comprising an antibody or antigen-binding fragment thereof capable of specifically binding to a TCR Vβ region, comprising a (i) heavy chain variable region (VH) having an amino acid sequence according to any of SEQ ID NOs: 228-295 and 559-569, and (ii) a light chain variable region (VL) having an amino acid sequence according to any of SEQ ID NOs: 296-369 and 570-583.

In some aspects of the methods described herein, the antibody or antigen-binding fragment for example, comprises (i) a variable heavy chain (VH) which comprises: (a) CDR1, CDR2 and CDR3 according to SEQ ID NO:10, SEQ ID NO:11, and SEQ ID NO:12, respectively; or (b) CDR1, CDR2 and CDR3 according to SEQ ID NO:370, SEQ ID NO:371, and SEQ ID NO:372, respectively; or (c) CDR1, CDR2 and CDR3 according to SEQ ID NO:373, SEQ ID NO:374, and SEQ ID NO:375 respectively; or (d) CDR1, CDR2 and CDR3 according to SEQ ID NO:376, SEQ ID NO:377, and SEQ ID NO:378, respectively; or (e) CDR1, CDR2 and CDR3 according to SEQ ID NO:379, SEQ ID NO:380, and SEQ ID NO:381 respectively; or (f) CDR1, CDR2 and CDR3 according to SEQ ID NO:382, SEQ ID NO:383, and SEQ ID NO:384, respectively; or (g) CDR1, CDR2 and CDR3 according to SEQ ID NO:385, SEQ ID NO:386, and SEQ ID NO:387, respectively; or (h) CDR1, CDR2 and CDR3 according to SEQ ID NO:388, SEQ ID NO:389, and SEQ ID NO:390, respectively; or (i) CDR1, CDR2 and CDR3 according to SEQ ID NO:391, SEQ ID NO:392, and SEQ ID NO:393, respectively; or (j) CDR1, CDR2 and CDR3 according to SEQ ID NO:394, SEQ ID NO:395, and SEQ ID NO:396, respectively; or (k) CDR1, CDR2 and CDR3 according to SEQ ID NO:397, SEQ ID NO:398, and SEQ ID NO:399, respectively; or (l) CDR1, CDR2 and CDR3 according to SEQ ID NO:400, SEQ ID NO:401, and SEQ ID NO:402, respectively; or (m) CDR1, CDR2 and CDR3 according to SEQ ID NO:403, SEQ ID NO:404, and SEQ ID NO:405, respectively; or (n) CDR1, CDR2 and CDR3 according to SEQ ID NO:406, SEQ ID NO:407, and SEQ ID NO:408, respectively; or (o) CDR1, CDR2 and CDR3 according to SEQ ID NO:409, SEQ ID NO:410, and SEQ ID NO:411, respectively; or (p) CDR1, CDR2 and CDR3 according to SEQ ID NO:412, SEQ ID NO:413, and SEQ ID NO:414, respectively; or (q) CDR1, CDR2 and CDR3 according to SEQ ID NO:415, SEQ ID NO:416, and SEQ ID NO:417, respectively; or (r) CDR1, CDR2 and CDR3 according to SEQ ID NO:584, SEQ ID NO:585, and SEQ ID NO:586, respectively; or (s) CDR1, CDR2 and CDR3 according to SEQ ID NO:587, SEQ ID NO:588, and SEQ ID NO:589, respectively; or (t) CDR1, CDR2 and CDR3 according to SEQ ID NO:590, SEQ ID NO:591, and SEQ ID NO:592, respectively; or (u) CDR1, CDR2 and CDR3 according to SEQ ID NO:593, SEQ ID NO:594, and SEQ ID NO:595, respectively; and/or (ii) a variable light chain (VL) which comprises: (a) CDR1, CDR2 and CDR3 according to SEQ ID NO:13, SEQ ID NO:14, SEQ ID NO:15, respectively; or (b) CDR1, CDR2 and CDR3 according to SEQ ID NO:418, SEQ ID NO:419, SEQ ID NO:420, respectively; or (c) CDR1, CDR2 and CDR3 according to SEQ ID NO:421, SEQ ID NO:422, SEQ ID NO:423, respectively; or (d) CDR1, CDR2 and CDR3 according to SEQ ID NO:424, SEQ ID NO:425, SEQ ID NO:426, respectively; or (e) CDR1, CDR2 and CDR3 according to SEQ ID NO:427, SEQ ID NO:428, SEQ ID NO:429, respectively; or (f) CDR1, CDR2 and CDR3 according to SEQ ID NO:430, SEQ ID NO:431, SEQ ID NO:432, respectively; or (g) CDR1, CDR2 and CDR3 according to SEQ ID NO:433, SEQ ID NO:434, SEQ ID NO:435, respectively; or (h) CDR1, CDR2 and CDR3 according to SEQ ID NO:436, SEQ ID NO:437, SEQ ID NO:438, respectively; or (i) CDR1, CDR2 and CDR3 according to SEQ ID NO:439, SEQ ID NO:440, SEQ ID NO:441, respectively; or (j) CDR1, CDR2 and CDR3 according to SEQ ID NO:442, SEQ ID NO:443, SEQ ID NO:444, respectively; or (k) CDR1, CDR2 and CDR3 according to SEQ ID NO:445, SEQ ID NO:446, SEQ ID NO:447, respectively; or (1) CDR1, CDR2 and CDR3 according to SEQ ID NO:448, SEQ ID NO:449, SEQ ID NO:450, respectively; or (m) CDR1, CDR2 and CDR3 according to SEQ ID NO:451, SEQ ID NO:452, SEQ ID NO:453, respectively; or (n) CDR1, CDR2 and CDR3 according to SEQ ID NO:454, SEQ ID NO:455, SEQ ID NO:456, respectively; or (o) CDR1, CDR2 and CDR3 according to SEQ ID NO:457, SEQ ID NO:458, SEQ ID NO:459, respectively; or (p) CDR1, CDR2 and CDR3 according to SEQ ID NO:460, SEQ ID NO:461, SEQ ID NO:462, respectively; or (q) CDR1, CDR2 and CDR3 according to SEQ ID NO:463, SEQ ID NO:464, SEQ ID NO:465, respectively; or (r) CDR1, CDR2 and CDR3 according to SEQ ID NO:596, SEQ ID NO:597, SEQ ID NO:598, respectively; or (s) CDR1, CDR2 and CDR3 according to SEQ ID NO:599, SEQ ID NO:601, SEQ ID NO:601, respectively; or (t) CDR1, CDR2 and CDR3 according to SEQ ID NO:602, SEQ ID NO:603, SEQ ID NO:604, respectively; or (u) CDR1, CDR2 and CDR3 according to SEQ ID NO:605, SEQ ID NO:606, SEQ ID NO:607, respectively, or SEQ ID NO: 608, SEQ ID NO: 609, SEQ ID NO: 610, respectively.

In some aspects of the methods described herein, the variable heavy chain comprises an amino acid sequence having at least 85% sequence identity to any one of SEQ ID NOs: 228-295 and 559-569. In some aspects described herein, the variable heavy chain comprises an amino acid sequence having at least 86% sequence identity to any one of SEQ ID NOs: 228-295 and 559-569. In some aspects described herein, the variable heavy chain comprises an amino acid sequence having at least 87% sequence identity to any one of SEQ ID NOs: 228-295 and 559-569. In some aspects described herein, the variable heavy chain comprises an amino acid sequence having at least 88% sequence identity to any one of SEQ ID NOs: 228-295 and 559-569. In some aspects described herein, the variable heavy chain comprises an amino acid sequence having at least 89% sequence identity to any one of SEQ ID NOs: 228-295 and 559-569. In some aspects described herein, the variable heavy chain comprises an amino acid sequence having at least 90% sequence identity to any one of SEQ ID NOs: 228-295 and 559-569. In some aspects described herein, the variable heavy chain comprises an amino acid sequence having at least 91% sequence identity to any one of SEQ ID NOs: 228-295 and 559-569. In some aspects described herein, the variable heavy chain comprises an amino acid sequence having at least 92% sequence identity to any one of SEQ ID NOs: 228-295 and 559-569. In some aspects described herein, the variable heavy chain comprises an amino acid sequence having at least 93% sequence identity to any one of SEQ ID NOs: 228-295 and 559-569. In some aspects described herein, the variable heavy chain comprises an amino acid sequence having at least 94% sequence identity to any one of SEQ ID NOs: 228-295 and 559-569. In some aspects described herein, the variable heavy chain comprises an amino acid sequence having at least 95% sequence identity to any one of SEQ ID NOs: 228-295 and 559-569. In some aspects described herein, the variable heavy chain comprises an amino acid sequence having at least 96% sequence identity to any one of SEQ ID NOs: 228-295 and 559-569. In some aspects described herein, the variable heavy chain comprises an amino acid sequence having at least 97% sequence identity to any one of SEQ ID NOs: 228-295 and 559-569. In some aspects described herein, the variable heavy chain comprises an amino acid sequence having at least 98% sequence identity to any one of SEQ ID NOs: 228-295 and 559-569. In some aspects described herein, the variable heavy chain comprises an amino acid sequence having at least 99% sequence identity to any one of SEQ ID NOs: 228-295 and 559-569. In some aspects described herein, the variable heavy chain comprises an amino acid sequence according to any one of SEQ ID NOs: 228-295 and 559-569.

In some aspects of the methods described herein, the antibody or antigen-binding fragment comprising a heavy chain variable region (VH) as described above, further comprises a light chain variable region (VL) having an amino acid sequence according to any of SEQ ID NOs: 296-369 and 570-583.

In some aspects of the methods described herein, the variable light chain comprises an amino acid sequence having at least 85% sequence identity to any one of SEQ ID NOs: 296-369 and 570-583. In some aspects described herein, the variable light chain comprises an amino acid sequence having at least 86% sequence identity to any one of SEQ ID NOs: 296-369 and 570-583. In some aspects described herein, the variable light chain comprises an amino acid sequence having at least 87% sequence identity to any one of SEQ ID NOs: 296-369 and 570-583. In some aspects described herein, the variable light chain comprises an amino acid sequence having at least 88% sequence identity to any one of SEQ ID NOs: 296-369 and 570-583. In some aspects described herein, the variable light chain comprises an amino acid sequence having at least 89% sequence identity to any one of SEQ ID NOs: 296-369 and 570-583. In some aspects described herein, the variable light chain comprises an amino acid sequence having at least 90% sequence identity to any one of SEQ ID NOs: 296-369 and 570-583. In some aspects described herein, the variable light chain comprises an amino acid sequence having at least 91% sequence identity to any one of SEQ ID NOs: 296-369 and 570-583. In some aspects described herein, the variable light chain comprises an amino acid sequence having at least 92% sequence identity to any one of SEQ ID NOs: 296-369 and 570-583. In some aspects described herein, the variable light chain comprises an amino acid sequence having at least 93% sequence identity to any one of SEQ ID NOs: 296-369 and 570-583. In some aspects described herein, the variable light chain comprises an amino acid sequence having at least 94% sequence identity to any one of SEQ ID NOs: 296-369 and 570-583. In some aspects described herein, the variable light chain comprises an amino acid sequence having at least 95% sequence identity to any one of SEQ ID NOs: 296-369 and 570-583. In some aspects described herein, the variable light chain comprises an amino acid sequence having at least 96% sequence identity to any one of SEQ ID NOs: 296-369 and 570-583. In some aspects described herein, the variable light chain comprises an amino acid sequence having at least 97% sequence identity to any one of SEQ ID NOs: 296-369 and 570-583. In some aspects described herein, the variable light chain comprises an amino acid sequence having at least 98% sequence identity to any one of SEQ ID NOs: 296-369 and 570-583. In some aspects described herein, the variable light chain comprises an amino acid sequence having at least 99% sequence identity to any one of SEQ ID NOs: 296-369 and 570-583. In some aspects described herein, the variable light chain comprises an amino acid sequence according to any one of SEQ ID NOs: 296-369 and 570-583.

In some aspects of the methods described herein the antibody or antigen-binding fragment comprising a light chain variable region (VL) as described above, further comprises a heavy chain variable region (VH) having an amino acid sequence according to any of SEQ ID NOs: 228-295 and 559-569.

In some aspects of the methods described herein, the variable heavy chain comprises an amino acid sequence having any percent identity described above to any one of SEQ ID NOs: 228-295 and 559-569, and the variable light chain comprises an amino acid sequence having any percent identity described above to any one of SEQ ID NOs: 296-369 and 570-583. To illustrate, a non-limiting example pairing of variable heavy and light chains includes a variable heavy chain comprising an amino acid sequence having at least 88% sequence identity to any one of SEQ ID NOs: 228-295 and 559-569, and a variable light chain comprising an amino acid sequence having at least 93% sequence identity to any one of SEQ ID NOs: 296-369 and 570-583. In other aspects of the methods decribed herein, pairing of variable heavy and light chains includes a variable heavy chain comprising an amino acid sequence having any percent identity described above to any one of SEQ ID NOs: 228-295 and 559-569, and a variable light chain comprising an amino acid sequence according to any one of SEQ ID NOs: 296-369 and 570-583. In other aspects described herein, pairing of variable heavy and light chains includes a variable heavy chain comprising an amino acid sequence according to any one of SEQ ID NOs: 228-295 and 559-569, and a variable light chain comprising an amino acid sequence having any percent identity described above to any one of SEQ ID NOs: 296-369 and 570-583. To illustrate, a non-limiting example pairing of variable heavy and light chains includes a variable heavy chain comprising an amino acid sequence having at least 92% sequence identity to any one of SEQ ID NOs: 228-295 and 559-569, and a variable light chain comprising an amino acid sequence according to any one of SEQ ID NOs: 296-369 and 570-583. In yet other aspects described herein, the variable heavy chain comprises an amino acid sequence according to any one of SEQ ID NOs: 228-295 and 559-569, and the variable light chain comprises an amino acid sequence according to any one of SEQ ID NOs: 296-369 and 570-583.

In some aspects of the methods described herein, the antibody or antigen-binding fragment thereof is capable of specifically binding to TCR Vβ2 and, for example comprises (i) a variable heavy chain (VH), and/or a variable light chain (VL). In some aspects, the Vβ2 antibody or antigen binding fragment, for example, comprises (i) a variable heavy chain (VH) according to SEQ ID NO: 228, and/or (ii) a variable light chain (VL) according to SEQ ID NO: 296; SEQ ID NO: 297; SEQ ID NO: 298; SEQ ID NO: 299; SEQ ID NO: 300; SEQ ID NO: 301; SEQ ID NO: 302; or SEQ ID NO: 303. In some aspects, the Vβ2 antibody or antigen binding fragment, for example, comprises (i) a variable heavy chain (VH) according to SEQ ID NO: 229, and/or (ii) a variable light chain (VL) according to SEQ ID NO: 296; SEQ ID NO: 297; SEQ ID NO: 298; SEQ ID NO: 299; SEQ ID NO: 300; SEQ ID NO: 301; SEQ ID NO: 302; or SEQ ID NO: 303. In some aspects, the Vβ2 antibody or antigen binding fragment, for example, comprises (i) a variable heavy chain (VH) according to SEQ ID NO: 230, and/or (ii) a variable light chain (VL) according to SEQ ID NO: 296; SEQ ID NO: 297; SEQ ID NO: 298; SEQ ID NO: 299; SEQ ID NO: 300; SEQ ID NO: 301; SEQ ID NO: 302; or SEQ ID NO: 303. In some aspects, the Vβ2 antibody or antigen binding fragment, for example, comprises (i) a variable heavy chain (VH) according to SEQ ID NO: 231, and/or (ii) a variable light chain (VL) according to SEQ ID NO: 296; SEQ ID NO: 297; SEQ ID NO: 298; SEQ ID NO: 299; SEQ ID NO: 300; SEQ ID NO: 301; SEQ ID NO: 302; or SEQ ID NO: 303. In some aspects, the Vβ2 antibody or antigen binding fragment, for example, comprises (i) a variable heavy chain (VH) according to SEQ ID NO: 232, and/or (ii) a variable light chain (VL) according to SEQ ID NO: 296; SEQ ID NO: 297; SEQ ID NO: 298; SEQ ID NO: 299; SEQ ID NO: 300; SEQ ID NO: 301; SEQ ID NO: 302; or SEQ ID NO: 303. In some aspects, the Vβ2 antibody or antigen binding fragment, for example, comprises (i) a variable heavy chain (VH) according to SEQ ID NO: 233, and/or (ii) a variable light chain (VL) according to SEQ ID NO: 296; SEQ ID NO: 297; SEQ ID NO: 298; SEQ ID NO: 299; SEQ ID NO: 300; SEQ ID NO: 301; SEQ ID NO: 302; or SEQ ID NO: 303. In some aspects, the Vβ2 antibody or antigen binding fragment, for example, comprises (i) a variable heavy chain (VH) according to SEQ ID NO: 234, and/or (ii) a variable light chain (VL) according to SEQ ID NO: 296; SEQ ID NO: 297; SEQ ID NO: 298; SEQ ID NO: 299; SEQ ID NO: 300; SEQ ID NO: 301; SEQ ID NO: 302; or SEQ ID NO: 303. In some aspects, the Vβ2 antibody or antigen binding fragment, for example, comprises (i) a variable heavy chain (VH) according to SEQ ID NO: 235, and/or (ii) a variable light chain (VL) according to SEQ ID NO: 296; SEQ ID NO: 297; SEQ ID NO: 298; SEQ ID NO: 299; SEQ ID NO: 300; SEQ ID NO: 301; SEQ ID NO: 302; or SEQ ID NO: 303. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen binding fragment thereof is capable of specifically binding to Vβ7.2 and, for example comprises (i) a variable heavy chain (VH), and/or a variable light chain (VL). In some aspects, the Vβ7.2 antibody or antigen binding fragment, for example, comprises (i) a variable heavy chain (VH) according to SEQ ID NO: 236, and/or (ii) a variable light chain (VL) according to SEQ ID NO: 304; SEQ ID NO: 305; SEQ ID NO: 306; or SEQ ID NO: 307. In some aspects, the Vβ7.2 antibody or antigen binding fragment, for example, comprises (i) a variable heavy chain (VH) according to SEQ ID NO: 237, and/or (ii) a variable light chain (VL) according to SEQ ID NO: 304; SEQ ID NO: 305; SEQ ID NO: 306; or SEQ ID NO: 307. In some aspects, the Vβ7.2 antibody or antigen binding fragment, for example, comprises (i) a variable heavy chain (VH) according to SEQ ID NO: 238, and/or (ii) a variable light chain (VL) according to SEQ ID NO: 304; SEQ ID NO: 305; SEQ ID NO: 306; or SEQ ID NO: 307. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen binding fragment thereof is capable of specifically binding to Vβ4, and, for example comprises (i) a variable heavy chain (VH), and/or a variable light chain (VL). In some aspects, the Vβ4 antibody or antigen binding fragment, for example, comprises (i) a variable heavy chain (VH) according to SEQ ID NO: 239, and/or (ii) a variable light chain (VL) according to SEQ ID NO: 308; or SEQ ID NO: 309. In some aspects, the Vβ4 antibody or antigen binding fragment, for example, comprises (i) a variable heavy chain (VH) according to SEQ ID NO: 240, and/or (ii) a variable light chain (VL) according to SEQ ID NO: 308; or SEQ ID NO: 309. In some aspects, the Vβ4 antibody or antigen binding fragment, for example, comprises (i) a variable heavy chain (VH) according to SEQ ID NO: 241, and/or (ii) a variable light chain (VL) according to SEQ ID NO: 308; or SEQ ID NO: 309. In some aspects, the V134 antibody or antigen binding fragment, for example, comprises (i) a variable heavy chain (VH) according to SEQ ID NO: 242, and/or (ii) a variable light chain (VL) according to SEQ ID NO: 308; or SEQ ID NO: 309. In some aspects, the Vβ4 antibody or antigen binding fragment, for example, comprises (i) a variable heavy chain (VH) according to SEQ ID NO: 243, and/or (ii) a variable light chain (VL) according to SEQ ID NO: 308; or SEQ ID NO: 309. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen binding fragment thereof is capable of specifically binding to Vβ13.2 and, for example comprises (i) a variable heavy chain (VH), and/or a variable light chain (VL), In some aspects described herein, the antibody or antigen binding fragment thereof is capable of specifically binding to Vβ13.2 and, for example, comprises (i) a variable heavy chain (VH) according to SEQ ID NO: 244, and/or (ii) a variable light chain (VL) according to SEQ ID NO: 310; SEQ ID NO: 311; SEQ ID NO: 312; SEQ ID NO: 313; SEQ ID NO: 314; SEQ ID NO: 315; SEQ ID NO: 316; or SEQ ID NO: 317. In some aspects, the Vβ13.2 antibody or antigen binding fragment, for example, comprises (i) a variable heavy chain (VH) according to SEQ ID NO: 245, and/or (ii) a variable light chain (VL) according to SEQ ID NO: 310; SEQ ID NO: 311; SEQ ID NO: 312; SEQ ID NO: 313; SEQ ID NO: 314; SEQ ID NO: 315; SEQ ID NO: 316; or SEQ ID NO: 317. In some aspects, the Vβ13.2 antibody or antigen binding fragment, for example, comprises (i) a variable heavy chain (VH) according to SEQ ID NO: 246, and/or (ii) a variable light chain (VL) according to SEQ ID NO: 310; SEQ ID NO: 311; SEQ ID NO: 312; SEQ ID NO: 313; SEQ ID NO: 314; SEQ ID NO: 315; SEQ ID NO: 316; or SEQ ID NO: 317. In some aspects, the Vβ13.2 antibody or antigen binding fragment, for example, comprises (i) a variable heavy chain (VH) according to SEQ ID NO: 247, and/or (ii) a variable light chain (VL) according to SEQ ID NO: 310; SEQ ID NO: 311; SEQ ID NO: 312; SEQ ID NO: 313; SEQ ID NO: 314; SEQ ID NO: 315; SEQ ID NO: 316; or SEQ ID NO: 317. In some aspects, the Vβ13.2 antibody or antigen binding fragment, for example, comprises (i) a variable heavy chain (VH) according to SEQ ID NO: 248, and/or (ii) a variable light chain (VL) according to SEQ ID NO: 310; SEQ ID NO: 311; SEQ ID NO: 312; SEQ ID NO: 313; SEQ ID NO: 314; SEQ ID NO: 315; SEQ ID NO: 316; or SEQ ID NO: 317. In some aspects, the Vβ13.2 antibody or antigen binding fragment, for example, comprises (i) a variable heavy chain (VH) according to SEQ ID NO: 249, and/or (ii) a variable light chain (VL) according to SEQ ID NO: 310; SEQ ID NO: 311; SEQ ID NO: 312; SEQ ID NO: 313; SEQ ID NO: 314; SEQ ID NO: 315; SEQ ID NO: 316; or SEQ ID NO: 317. In some aspects, the Vβ13.2 antibody or antigen binding fragment, for example, comprises (i) a variable heavy chain (VH) according to SEQ ID NO: 250, and/or (ii) a variable light chain (VL) according to SEQ ID NO: 310; SEQ ID NO: 311; SEQ ID NO: 312; SEQ ID NO: 313; SEQ ID NO: 314; SEQ ID NO: 315; SEQ ID NO: 316; or SEQ ID NO: 317. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen binding fragment thereof is capable of specifically binding to V814 and, for example comprises (i) a variable heavy chain (VH), and/or a variable light chain (VL). In some aspects, the V814 antibody or antigen binding fragment, for example, comprises (i) a variable heavy chain (VH) according to SEQ ID NO: 251, and/or (ii) a variable light chain (VL) according to SEQ ID NO: 318; or SEQ ID NO: 319. SEQ ID NO: 320; SEQ ID NO: 321; SEQ ID NO: 322; or SEQ ID NO: 323. In some aspects, the V814 antibody or antigen binding fragment, for example, comprises (i) a variable heavy chain (VH) according to SEQ ID NO: 252, and/or (ii) a variable light chain (VL) according to SEQ ID NO: 318; or SEQ ID NO: 319. SEQ ID NO: 320; SEQ ID NO: 321; SEQ ID NO: 322; or SEQ ID NO: 323. In some aspects, the V814 antibody or antigen binding fragment, for example, comprises (i) a variable heavy chain (VH) according to SEQ ID NO: 253, and/or (ii) a variable light chain (VL) according to SEQ ID NO: 318; or SEQ ID NO: 319. SEQ ID NO: 320; SEQ ID NO: 321; SEQ ID NO: 322; or SEQ ID NO: 323. In some aspects, the V814 antibody or antigen binding fragment, for example, comprises (i) a variable heavy chain (VH) according to SEQ ID NO: 254, and/or (ii) a variable light chain (VL) according to SEQ ID NO: 318; or SEQ ID NO: 319. SEQ ID NO: 320; SEQ ID NO: 321; SEQ ID NO: 322; or SEQ ID NO: 323. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen binding fragment thereof is capable of specifically binding to Vβ22 and, for example comprises (i) a variable heavy chain (VH), and/or a variable light chain (VL). In some aspects, the Vβ22 antibody or antigen binding fragment, for example, comprises (i) a variable heavy chain (VH) according to SEQ ID NO: 255, and/or (ii) a variable light chain (VL) according to SEQ ID NO: 324; SEQ ID NO: 325; SEQ ID NO: 326; or SEQ ID NO: 327. In some aspects, the Vβ22 antibody or antigen binding fragment, for example, comprises (i) a variable heavy chain (VH) according to SEQ ID NO: 256, and/or (ii) a variable light chain (VL) according to SEQ ID NO: 324; SEQ ID NO: 325; SEQ ID NO: 326; or SEQ ID NO: 327. In some aspects, the Vβ22 antibody or antigen binding fragment, for example, comprises (i) a variable heavy chain (VH) according to SEQ ID NO: 257, and/or (ii) a variable light chain (VL) according to SEQ ID NO: 324; SEQ ID NO: 325; SEQ ID NO: 326; or SEQ ID NO: 327. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen binding fragment thereof is capable of specifically binding to Vβ11 and, for example comprises (i) a variable heavy chain (VH), and/or a variable light chain (VL). In some aspects, the Vβ11 antibody or antigen binding fragment, for example, comprises (i) a variable heavy chain (VH) according to SEQ ID NO: 258, and/or (ii) a variable light chain (VL) according to SEQ ID NO: 328; SEQ ID NO: 329; SEQ ID NO: 330; or SEQ ID NO: 331. In some aspects, the Vβ11 antibody or antigen binding fragment, for example, comprises (i) a variable heavy chain (VH) according to SEQ ID NO: 259, and/or (ii) a variable light chain (VL) according to SEQ ID NO: 328; SEQ ID NO: 329; SEQ ID NO: 330; or SEQ ID NO: 331, In some aspects, the Vβ11 antibody or antigen binding fragment, for example, comprises (i) a variable heavy chain (VH) according to SEQ ID NO: 260, and/or (ii) a variable light chain (VL) according to SEQ ID NO: 328; SEQ ID NO: 329; SEQ ID NO: 330; or SEQ ID NO: 331. In some aspects, the Vβ11 antibody or antigen binding fragment, for example, comprises (i) a variable heavy chain (VH) according to SEQ ID NO: 261, and/or (ii) a variable light chain (VL) according to SEQ ID NO: 332; SEQ ID NO: 333; SEQ ID NO: 334; SEQ ID NO: 335; or SEQ ID NO: 336. In some aspects, the Vβ11 antibody or antigen binding fragment, for example, comprises (i) a variable heavy chain (VH) according to SEQ ID NO: 262, and/or (ii) a variable light chain (VL) according to SEQ ID NO: 332; SEQ ID NO: 333; SEQ ID NO: 334; SEQ ID NO: 335; or SEQ ID NO: 336. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen binding fragment thereof is capable of specifically binding to Vβ12 and, for example comprises (i) a variable heavy chain (VH), and/or a variable light chain (VL). In some aspects, the Vβ12 antibody or antigen binding fragment, for example, comprises (i) a variable heavy chain (VH) according to SEQ ID NO: 263, and/or (ii) a variable light chain (VL) according to SEQ ID NO: 337; SEQ ID NO: 338; SEQ ID NO: 339; SEQ ID NO: 340; or SEQ ID NO: 341. In some aspects, the Vβ12 antibody or antigen binding fragment, for example, comprises (i) a variable heavy chain (VH) according to SEQ ID NO: 264, and/or (ii) a variable light chain (VL) according to SEQ ID NO: 337; SEQ ID NO: 338; SEQ ID NO: 339; SEQ ID NO: 340; or SEQ ID NO: 341. In some aspects, the Vβ12 antibody or antigen binding fragment, for example, comprises (i) a variable heavy chain (VH) according to SEQ ID NO: 265, and/or (ii) a variable light chain (VL) according to SEQ ID NO: 337; SEQ ID NO: 338; SEQ ID NO: 339; SEQ ID NO: 340; or SEQ ID NO: 341. In some aspects, the Vβ12 antibody or antigen binding fragment, for example, comprises (i) a variable heavy chain (VH) according to SEQ ID NO: 266, and/or (ii) a variable light chain (VL) according to SEQ ID NO: 337; SEQ ID NO: 338; SEQ ID NO: 339; SEQ ID NO: 340; or SEQ ID NO: 341. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen binding fragment thereof is capable of specifically binding to Vβ8 and, for example comprises (i) a variable heavy chain (VH), and/or a variable light chain (VL). In some aspects, the Vβ8 antibody or antigen binding fragment, for example, comprises (i) a variable heavy chain (VH) according to SEQ ID NO: 267, and/or (ii) a variable light chain (VL) according to SEQ ID NO: 342; SEQ ID NO: 343; or SEQ ID NO: 344. In some aspects, the Vβ8 antibody or antigen binding fragment, for example, comprises (i) a variable heavy chain (VH) according to SEQ ID NO: 268, and/or (ii) a variable light chain (VL) according to SEQ ID NO: 342; SEQ ID NO: 343; or SEQ ID NO: 344. In some aspects, the Vβ8 antibody or antigen binding fragment, for example, comprises (i) a variable heavy chain (VH) according to SEQ ID NO: 269, and/or (ii) a variable light chain (VL) according to SEQ ID NO: 342; SEQ ID NO: 343; or SEQ ID NO: 344. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen binding fragment thereof is capable of specifically binding to Vβ13.1 and, for example comprises (i) a variable heavy chain (VH), and/or a variable light chain (VL). In some aspects, the Vβ13.1 antibody or antigen binding fragment, for example, comprises (i) a variable heavy chain (VH) according to SEQ ID NO: 270, and/or (ii) a variable light chain (VL) according to SEQ ID NO: 345; SEQ ID NO: 346; or SEQ ID NO: 347. In some aspects, the Vβ13.1 antibody or antigen binding fragment, for example, comprises (i) a variable heavy chain (VH) according to SEQ ID NO: 271, and/or (ii) a variable light chain (VL) according to SEQ ID NO: 345; SEQ ID NO: 346; or SEQ ID NO: 347. In some aspects, the Vβ13.1 antibody or antigen binding fragment, for example, comprises (i) a variable heavy chain (VH) according to SEQ ID NO: 272, and/or (ii) a variable light chain (VL) according to SEQ ID NO: 345; SEQ ID NO: 346; or SEQ ID NO: 347. In some aspects, the Vβ13.1 antibody or antigen binding fragment, for example, comprises (i) a variable heavy chain (VH) according to SEQ ID NO: 273, and/or (ii) a variable light chain (VL) according to SEQ ID NO: 345; SEQ ID NO: 346; or SEQ ID NO: 347. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen binding fragment thereof is capable of specifically binding to Vβ5.1 and, for example comprises (i) a variable heavy chain (VH), and/or a variable light chain (VL). In some aspects, the Vβ5.1 antibody or antigen binding fragment, for example, comprises (i) a variable heavy chain (VH) according to SEQ ID NO: 274, and/or (ii) a variable light chain (VL) according to SEQ ID NO: 348; SEQ ID NO: 349; or SEQ ID NO: 350. In some aspects, the Vβ5.1 antibody or antigen binding fragment, for example, comprises (i) a variable heavy chain (VH) according to SEQ ID NO: 275, and/or (ii) a variable light chain (VL) according to SEQ ID NO: 348; SEQ ID NO: 349; or SEQ ID NO: 350. In some aspects, the Vβ5.1 antibody or antigen binding fragment, for example, comprises (i) a variable heavy chain (VH) according to SEQ ID NO: 276, and/or (ii) a variable light chain (VL) according to SEQ ID NO: 348; SEQ ID NO: 349; or SEQ ID NO: 350. In some aspects, the Vβ5.1 antibody or antigen binding fragment, for example, comprises (i) a variable heavy chain (VH) according to SEQ ID NO: 277, and/or (ii) a variable light chain (VL) according to SEQ ID NO: 348; SEQ ID NO: 349; or SEQ ID NO: 350. In some aspects, the Vβ5.1 antibody or antigen binding fragment, for example, comprises (i) a variable heavy chain (VH) according to SEQ ID NO: 278, and/or (ii) a variable light chain (VL) according to SEQ ID NO: 348; SEQ ID NO: 349; or SEQ ID NO: 350. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen binding fragment thereof is capable of specifically binding to Vβ9 and, for example comprises (i) a variable heavy chain (VH), and/or a variable light chain (VL). In some aspects, the Vβ9 antibody or antigen binding fragment, for example, comprises (i) a variable heavy chain (VH) according to SEQ ID NO: 279, and/or (ii) a variable light chain (VL) according to SEQ ID NO: 351; SEQ ID NO: 352; SEQ ID NO: 353; SEQ ID NO: 354. In some aspects, the Vβ9 antibody or antigen binding fragment, for example, comprises (i) a variable heavy chain (VH) according to SEQ ID NO: 280, and/or (ii) a variable light chain (VL) according to SEQ ID NO: 351; SEQ ID NO: 352; SEQ ID NO: 353; or SEQ ID NO: 354, In some aspects, the Vβ9 antibody or antigen binding fragment, for example, comprises (i) a variable heavy chain (VH) according to SEQ ID NO: 281, and/or (ii) a variable light chain (VL) according to SEQ ID NO: 351; SEQ ID NO: 352; SEQ ID NO: 353; or SEQ ID NO: 354. In some aspects, the Vβ9 antibody or antigen binding fragment, for example, comprises (i) a variable heavy chain (VH) according to SEQ ID NO: 282, and/or (ii) a variable light chain (VL) according to SEQ ID NO: 351; SEQ ID NO: 352; SEQ ID NO: 353; or SEQ ID NO: 354. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen binding fragment thereof is capable of specifically binding to Vβ17 and, for example comprises (i) a variable heavy chain (VH), and/or a variable light chain (VL). In some aspects, the Vβ17 antibody or antigen binding fragment, for example, comprises (i) a variable heavy chain (VH) according to SEQ ID NO: 283, and/or (ii) a variable light chain (VL) according to SEQ ID NO: 355; SEQ ID NO: 356; or SEQ ID NO: 357. In some aspects, the Vβ17 antibody or antigen binding fragment, for example, comprises (i) a variable heavy chain (VH) according to SEQ ID NO: 284, and/or (ii) a variable light chain (VL) according to SEQ ID NO: 355; SEQ ID NO: 356; or SEQ ID NO: 357. In some aspects, the Vβ17 antibody or antigen binding fragment, for example, comprises (i) a variable heavy chain (VH) according to SEQ ID NO: 285, and/or (ii) a variable light chain (VL) according to SEQ ID NO: 355; SEQ ID NO: 356; or SEQ ID NO: 357. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen binding fragment thereof is capable of specifically binding to Vβ3 and, for example comprises (i) a variable heavy chain (VH), and/or a variable light chain (VL). In some aspects, the Vβ3 antibody or antigen binding fragment, for example, comprises (i) a variable heavy chain (VH) according to SEQ ID NO: 286, and/or (ii) a variable light chain (VL) according to SEQ ID NO: 358; SEQ ID NO: 359; or SEQ ID NO: 360. In some aspects, the Vβ3 antibody or antigen binding fragment, for example, comprises (i) a variable heavy chain (VH) according to SEQ ID NO: 287, and/or (ii) a variable light chain (VL) according to SEQ ID NO: 358; SEQ ID NO: 359; or SEQ ID NO: 360. In some aspects, the Vβ3 antibody or antigen binding fragment, for example, comprises (i) a variable heavy chain (VH) according to SEQ ID NO: 289, and/or (ii) a variable light chain (VL) according to SEQ ID NO: 358; SEQ ID NO: 359; or SEQ ID NO: 360. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen binding fragment thereof is capable of specifically binding to Vβ7.1 and, for example comprises (i) a variable heavy chain (VH), and/or a variable light chain (VL). In some aspects, the Vβ7.1 antibody or antigen binding fragment, for example, comprises (i) a variable heavy chain (VH) according to SEQ ID NO: 290, and/or (ii) a variable light chain (VL) according to SEQ ID NO: 361; SEQ ID NO: 362; SEQ ID NO: 363; SEQ ID NO: 364; SEQ ID NO: 365; SEQ ID NO: 366; or SEQ ID NO: 367. In some aspects, the Vβ7.1 antibody or antigen binding fragment, for example, comprises (i) a variable heavy chain (VH) according to SEQ ID NO: 291, and/or (ii) a variable light chain (VL) according to SEQ ID NO: 361; SEQ ID NO: 362; SEQ ID NO: 363; SEQ ID NO: 364; SEQ ID NO: 365; SEQ ID NO: 366; or SEQ ID NO: 367. In some aspects, the Vβ7.1 antibody or antigen binding fragment, for example, comprises (i) a variable heavy chain (VH) according to SEQ ID NO: 292, and/or (ii) a variable light chain (VL) according to SEQ ID NO: 361; SEQ ID NO: 362; SEQ ID NO: 363; SEQ ID NO: 364; SEQ ID NO: 365; SEQ ID NO: 366; or SEQ ID NO: 367. In some aspects, the Vβ7.1 antibody or antigen binding fragment, for example, comprises (i) a variable heavy chain (VH) according to SEQ ID NO: 293, and/or (ii) a variable light chain (VL) according to SEQ ID NO: 361; SEQ ID NO: 362; SEQ ID NO: 363; SEQ ID NO: 364; SEQ ID NO: 365; SEQ ID NO: 366; or SEQ ID NO: 367. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen binding fragment thereof is capable of specifically binding to Vβ5.3 and, for example comprises (i) a variable heavy chain (VH), and/or a variable light chain (VL). In some aspects, the Vβ5.3 antibody or antigen binding fragment, for example, comprises (i) a variable heavy chain (VH) according to SEQ ID NO: 294, and/or (ii) a variable light chain (VL) according to SEQ ID NO: 368 or SEQ ID NO: 369. In some aspects, the Vβ5.3 antibody or antigen binding fragment, for example, comprises (i) a variable heavy chain (VH) according to SEQ ID NO: 295, and/or (ii) a variable light chain (VL) according to SEQ ID NO: 368 or SEQ ID NO: 369. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen binding fragment thereof is capable of specifically binding to Vβ20 and, for example comprises (i) a variable heavy chain (VH), and/or a variable light chain (VL). In some aspects, the Vβ20 antibody or antigen binding fragment, for example, comprises (i) a variable heavy chain (VH) according to SEQ ID NO: 559, and/or (ii) a variable light chain (VL) according to SEQ ID NO: 570; SEQ ID NO: 571; or SEQ ID NO: 572. In some aspects, the Vβ20 antibody or antigen binding fragment, for example, comprises (i) a variable heavy chain (VH) according to SEQ ID NO: 560, and/or (ii) a variable light chain (VL) according to SEQ ID NO: 570; SEQ ID NO: 571; or SEQ ID NO: 572. In some aspects, the Vβ20 antibody or antigen binding fragment, for example, comprises (i) a variable heavy chain (VH) according to SEQ ID NO: 561, and/or (ii) a variable light chain (VL) according to SEQ ID NO: 570; SEQ ID NO: 571; or SEQ ID NO: 572. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen binding fragment thereof is capable of specifically binding to Vβ18 and, for example comprises (i) a variable heavy chain (VH), and/or a variable light chain (VL). In some aspects, the Vβ18 antibody or antigen binding fragment, for example, comprises (i) a variable heavy chain (VH) according to SEQ ID NO: 562, and/or (ii) a variable light chain (VL) according to SEQ ID NO: 573; SEQ ID NO: 574; SEQ ID NO: 575. In some aspects, the Vβ18 antibody or antigen binding fragment, for example, comprises (i) a variable heavy chain (VH) according to SEQ ID NO: 563, and/or (ii) a variable light chain (VL) according to SEQ ID NO: 573; SEQ ID NO: 574; SEQ ID NO: 575. In some aspects, the Vβ18 antibody or antigen binding fragment, for example, comprises (i) a variable heavy chain (VH) according to SEQ ID NO: 564, and/or (ii) a variable light chain (VL) according to SEQ ID NO: 573; SEQ ID NO: 574; SEQ ID NO: 575. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen binding fragment thereof is capable of specifically binding to Vβ13.6 and, for example comprises (i) a variable heavy chain (VH), and/or a variable light chain (VL). In some aspects, the Vβ13.6 antibody or antigen binding fragment, for example, comprises (i) a variable heavy chain (VH) according to SEQ ID NO: 565, and/or (ii) a variable light chain (VL) according to SEQ ID NO: 576; SEQ ID NO: 577. In some aspects, the Vβ13.6 antibody or antigen binding fragment, for example, comprises (i) a variable heavy chain (VH) according to SEQ ID NO: 566, and/or (ii) a variable light chain (VL) according to SEQ ID NO: 576; SEQ ID NO: 577. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen binding fragment thereof is capable of specifically binding to Vβ16 and, for example comprises (i) a variable heavy chain (VH), and/or a variable light chain (VL). In some aspects, the Vβ16 antibody or antigen binding fragment, for example, comprises (i) a variable heavy chain (VH) according to SEQ ID NO: 567, and/or (ii) a variable light chain (VL) according to SEQ ID NO: 578; SEQ ID NO: 579; SEQ ID NO: 580; SEQ ID NO: 581; SEQ ID NO: 582; or SEQ ID NO: 583. In some aspects, the Vβ16 antibody or antigen binding fragment, for example, comprises (i) a variable heavy chain (VH) according to SEQ ID NO: 568, and/or (ii) a variable light chain (VL) according to SEQ ID NO: 578; SEQ ID NO: 579; SEQ ID NO: 580; SEQ ID NO: 581; SEQ ID NO: 582; or SEQ ID NO: 583. In some aspects, the Vβ16 antibody or antigen binding fragment, for example, comprises (i) a variable heavy chain (VH) according to SEQ ID NO: 569, and/or (ii) a variable light chain (VL) according to SEQ ID NO: 578; SEQ ID NO: 579; SEQ ID NO: 580; SEQ ID NO: 581; SEQ ID NO: 582; or SEQ ID NO: 583. In some aspects, the VH and VL described above comprises an amino acid sequence having at least 85%, at least 90%, at least 91% at least 92%, at least 93%, at least 94%, at least 95%, at least 96%, at least 97%, at least 98%, at least 99%, or about 100% percent identity to any one SEQ ID NO. described above.

In some aspects of the methods described herein, the antibody or antigen-binding fragment, comprises a VH and a VL on the same polypeptide chain. In some aspects described herein, the antibody or antigen-binding fragment, comprises a VH and a VL on separate polypeptide chains.

In some aspects of the methods described herein, the TCR Vβ region is selected from the group consisting of Vβ1, Vβ2, Vβ3, Vβ4, Vβ5.1, Vβ5.3, Vβ6, Vβ7.1, Vβ7.2, Vβ8, Vβ9, Vβ10, Vβ11, Vβ12, Vβ13.1, Vβ13.2, Vβ14, Vβ15, Vβ16, Vβ17, Vβ18, Vβ19, Vβ20, Vβ21, Vβ22, Vβ23, Vβ24, Vβ25, Vβ26, Vβ27, Vβ28, Vβ29, Vβ30, VβA, VβB, and VβC. It will be understood that a combination of variable heavy and light chains described herein that specifically bind to the same TCR Vβ region are capable of providing increased in vitro and/or in vivo activity compared to a combination of variable heavy and light chains described herein that specifically bind to different TCR Vβ regions.

In some aspects of the methods described herein, the TCR Vβ region is selected from the group consisting of Vβ1, Vβ2, Vβ3, Vβ4, Vβ5.1, Vβ5.3, Vβ7.1, Vβ7.2, Vβ8, Vβ9, Vβ11, Vβ12, Vβ13.1, Vβ13.2, Vβ13.6, Vβ14, Vβ16, Vβ17, Vβ18, Vβ20, and Vβ22.

In some aspects of the methods described herein, the TCR Vβ region is Vβ2.

In some aspects of the methods described herein, the cancer is selected from the group consisting of T-cell Lymphoma, T-cell Leukemia, Cutaneous T-cell Lymphoma, Peripheral T-cell Lymphoma (PTCL), Not Otherwise Specified PTCL (PTCL-NOS), Angioimmunoblastic T-cell Lymphoma (AITL), Anaplastic Large-cell Lymphoma (ALCL), Enteropathy-Associated T-cell Lymphoma (EATL), Adult T-cell Leukemia/Lymphoma (ATLL), Hepatosplenic T-cell Lymphoma (HSTL), Subcutaneous Panniculitis-Like T-cell Lymphoma (SPTCL), T-cell Acute Lymphoblastic Leukemia (T-ALL), T-cell Chronic Lymphocytic Leukemia, Large Granular Lymphocyte Leukemia, T-cell Prolymphocytic Leukemia, Lympomatoid Papulosis, Small Medium Pleiomorphic T-cell Lymphoma, Mycosis Fungoides, Sezary Syndrome, and Cytotoxic T-cell Lymphoma.

In some aspects of the methods described herein, the T-cell mediated disease is selected from the group consisting of Atherosclerosis, Parkinson's disease, Alzheimer's disease, Type 1 Diabetes Mellitus, Grave's Disease, Hashimoto's Thyroiditis, Addison's Disease, Rheumatoid Arthritis (RA), Multiple Sclerosis (MS), Psoriasis, Psoriatic Arthritis, Lichen Planus, Lichen Planopilaris, Morphea, Scleroderma, Systemic Sclerosis, acute or chronic Graft-Versus-Host Disease (GVHD), Cardiac Organ Transplant Rejection, Pulmonary Organ Transplant Rejection, Renal Organ Transplant Rejection, Interstitial Lung Disease, Scleroderma, Alopecia Areata, Vitiligo, Celiac Disease (Sprue), Myasthenia Gravis, Pernicious Anemia, Sjogren's Syndrome, Systemic Lupus Erythematosus, Chronic Inflammatory Demyelinating Polyneuropathy, Guillain-Barre Syndrome, Inflammatory Bowel Disease, Crohn's Disease, and Ulcerative Colitis.

In some aspects of the methods described herein, the composition is delivered intratumorally, intravenously, intraperitoneally, intramuscularly, intrathecally, transdermally, or subcutaneously.

In some aspects, the composition is delivered intravenously or intratumorally.

In some aspects of the methods, described herein, the subject is a human.

In some aspects of the methods described herein, the administration of the CAR or antibody or antigen binding fragments can include, but are not limited to the co-administration or successive administration with standard of care (SOC) or other agents, including chemotherapy, small molecule targeted-therapies, immunotherapies or other immune modifying strategies (e.g., checkpoint inhibitor therapy, recombinant χΨτoκvε therapies, cellular therapies such as gamma-delata T cell and natural killer therapies), such as a combination therapy. In some aspects, the CAR and antibody and antigen binding fragment treatments can both be applicable for autoimmune, inflammatory disorders, and/or cancer treatment. In some aspects, they are not exclusive to one method of treatment or the other.

In some aspects of the methods described herein, the combination antibody and antigen binding fragment treatment targeting multiple Vβ families are preferentially effective for treating autoimmune and inflammatory disorders. In some aspects, the antibody and antigen binding fragment treatment targeting a single Vβ family are preferentially effective for treating T cell leukemia. In some aspects, the CAR treatment is preferentially effective for treating T cell lymphoma.

In some aspects of the methods described herein, the combination of antibodies and/or antigen binding fragments or or the combination of CAR therapies against more than one Vβ family are used to treat autoimmune and inflammatory disorders. Without wishing to be bound by any one theory, the combination of antibodies and/or antigen binding fragments or the combination of CAR therapies against more than one Vβ family may be necessary to eliminate multiple sets of autoreactive and disease-mediating T cells. In some aspects this combination therapy is used in a personalized medicine approach where patients are first screened, for example, for the most reactive T cell population as defined by their Vβ usage, and once the reactive T cell population is identified or determined, the patient is treated with the combination of antibodies and/or antigen binding fragments or or the combination of CAR therapies to target the patient's reactive T cell population. In some aspects, the methods disclosed herein can also extend beyond the treatment of T-cell malignancies, and include but are not limited to refined targeting of specific subsets of T-cells, which can be applied to any disease in which expansions of certain clonal T-cell subsets contribute to the disease, or disease process. In some aspects disclosed herein, is treating a T cell-associated disease in a subject in need thereof. In some aspects, the T cell-associated disease is an autoimmune disease wherein T cells are the mediators of the autoimmunity. In some aspects, the CAR, the antibody or antigen binding fragment, the composition of the disclosure, or the cells disclosed herein can be used to target a pathogenic T cell population responsible for an autoimmune disease.

In some aspects of the methods described herein, the subject afflicted with, suffering from, or at risk of a cancer or an autoimmune or inflammatory disorder can be assessed for treatment, or to not be treated, before initiating treatment according to the methods described herein or after initiating treatment according to the methods described herein. In some aspects, the subject can be assessed by determining the subject's TCR Vβ usage. In some aspects the most active and relevant autoreactive T cells as defined by a subject's TCR Vβ usage can be used. In some aspects, determining the subject's TCR Vβ usage can be performerd by flow cytometry of a sample from the subject to screen the subject. In some aspects that the subject's sample is blood sample, such as a peripheral blood sample. In some aspects that the subject's sample is a tissue from a biopsy. In some aspects, the subject's sample can include both a blood sample, such as a peripheral blood sample, and a tissue sample. In some aspects, determining the subject's TCR Vβ usage can be performerd by multicolor antibodies to identify TCR Vβ specificity. In some aspects the specificity of such multicolor antibodies include the same specificities as the CAR, antibody or antigen binding fragments, or compositions described herein. In some aspects these multicolor antibodies are capable of co-identifyig TCR Vβeta specificity via activated and/or effector T cells (via activation and subset defining markers). In some aspects, these multicolor antibodies are capable of identifying TCR Vβ and/or TCR specificity e.g. via MHC-peptide conjugates (e.g. tetramers, dendrimers). In some aspects, determining the subject's TCR Vβ usage can be performerd by a single use cell RNA sequencing or spatial omics to identify the relevant autoreactive clones. In some aspects this screening can be used to define which Vβ family or families are targeted by the CAR or antibody or antigen binding fragment described herein. In some aspects, similar screening approaches can be used to monitor effects of therapy by, for example, detecting the elimination of the autoreactive and pathogenic T cells, including as defined by their Vβ usage, according to any methods described herein. In some aspects, the methods described herein includes screening a subject undergoing treatment according to the methods described herein, which can also be used to redefine subsequent treatments, e.g. alter the Vβ families targeted and, thus, use of CAR or antibody or antigen binding fragments described herein.

In some aspects of the methods described herein, the administration of the CAR, the antibody or antigen binding fragment, the composition of the disclosure, or cells disclosed herein enhances the response (e.g., partial, complete, or poor response, respectively) to an anti-cancer treatment compared to treatment with the anti-cancer treatment alone. As examples, enhancing the response to an anti-cancer treatment can include one or more of shrinking tumor size (including a partial response or complete response), reducing the number of a cancer cells (e.g., hematologic cancer cells in a blood sample), diminishing/eliminating metastases, halting proliferation of cancer cells, lessening the risk of relapse, lowering a cancer's clinical stage and/or grade, increasing the expression of one or more biomarkers associated with a reduction in the number or proliferation of cancer cells, and/or improving quality of life indicators (including disease-free survival) when compared to treatment with the anti-cancer treatment alone.

In some aspects of the methods described herein, administering the CAR or antibody or antigen binding fragment, or the compositions disclosed herein provides a greater reduction in tumor grade, e.g., according to a Tumor; Node involvement, and Metastatic spread (TNM) staging system, than is reduced by the anti-cancer treatment alone. For example, over the course of a treatment, a stage IV tumor may be reduced to a stage II tumor, whereas a stage IV tumor may only be reduced to a stage III tumor over the course of a treatment only including the anti-cancer treatment. Similarly, administering the CAR or antibody or antigen binding fragment, or the compositions disclosed herein can provide greater reduction in cancer stage (which relates to how abnormal the cancer cells appear, how rapidly they proliferate, and how likely they are to metastasize) than by a method comprising the anti-cancer treatment alone. Also, the administration of the CAR or antibody or antigen binding fragment, or the compositions disclosed herein may shrink a tumor's size faster and to a greater extent than would a treatment only including the anti-cancer treatment.

In the context of administering a composition to reduce the risk of a poor response to CAR or antibody or antigen binding fragment treatment, or to increase the chance of a complete response or partial response to CAR or antibody or antigen binding fragment, an effective amount of a composition described herein is an amount sufficient to decrease the likelihood of a poor response or to increase the likelihood of a complete response or partial response by at 10%, 20%, 30%, 40%, 50%, 60%, 70%, 80%, 90%, 95%, 98% or 99%.

The CAR, the antibody or antigen binding fragment, the composition of the disclosure, or cells described herein, can be administered in a manner appropriate to the disease to be treated (or prevented). The amount and frequency of administration will be determined by factors such as the condition of the patient and the type and severity of the patient's disease, although clinical trials can determine appropriate doses.

In some aspects of the methods described herein, the administration of allogeneic CAR-T cells at an initial candidate dosage can be about 1.1 to about 2.3×10⁵ cells/kg CAR-T cells. For the purpose of the present disclosure, a typical flat dosage of CAR-T cells might range from about any of about 1×10⁴ to 1×10⁵ to 1×10⁶ to 1×10⁷, to 1×10⁷, to 1×10⁸ cells or more, depending on the factors mentioned above. For example, dosage of about 0.3×10⁴ cells, about 0.5×10⁴ cells, about 1×10⁴ cells, about 1.5×10⁴ cells, about 2×10⁴ cells, about 2.5×10⁴ cells, about 3×10⁴ cells, about 3.5×10⁴ cells, about 4×10⁴ cells, about 4.5×10⁴ cells, about 5×10⁴ cells, about 5.5×10⁴ cells, about 6×10⁴ cells, about 6.5×10⁴ cells, about 7×10⁴ cells, about 7.5×1×10⁴ cells, about 8×1×10⁴ cells, about 8.5×1×10⁴ cells, about 9×1×10⁴ cells, about 9.5×10⁴ cells, about 1×10⁵ cells, about 1.5×10⁵ cells, 2×10⁵ cells, about 2.5×10⁵ cells, about 3×10⁵ cells, about 3.5×10⁵ cells, about 4×10⁵ cells, about 4.5×10⁵ cells, about 5×10⁵ cells, about 5.5×10⁵ cells, about 6×10⁵ cells, about 6.5×10⁵ cells, about 7×10⁵ cells, about 7.5×10⁵ cells, about 8×10⁵ cells, about 8.5×10⁵ cells, about 9×10⁵ cells, about 9.5×10⁵ cells, about 1×10⁶ cells, about 1.5×10⁶ cells, 2×10⁶ cells, about 2.5×10⁶ cells, about 3×10⁶ cells, about 3.5×10⁶ cells, about 4×10⁶ cells, about 4.5×10⁶ cells, about 5×10⁶ cells, about 5.5×10⁶ cells, about 6×10⁶ cells, about 6.5×10⁶ cells, about 7×10⁶ cells, about 7.5×10⁶ cells, about 8×10⁶ cells, about 8.5×10⁶ cells, about 9×10⁶ cells, about 9.5×10⁶ cells, about 1×10⁷ cells, about 1.5×10⁷ cells, 2×10⁷ cells, about 2.5×10⁷ cells, about 3×10⁷ cells, about 3.5×10⁷ cells, about 4×10⁷ cells, about 4.5×10⁷ cells, about 5×10⁷ cells, about 5.5×10⁷ cells, about 6×10⁷ cells, about 6.5×10⁷ cells, about 7×10⁷ cells, about 7.5×10⁷ cells, about 8×10⁷ cells, about 8.5×10⁷ cells, about 9×10⁷ cells, about 9.5×10⁷ cells, about 1×10⁸ cells, about 1.5×10⁸ cells, 2×10⁸ cells, about 2.5×10⁸ cells, about 3×10⁸ cells, about 3.5×10⁸ cells, about 4×10⁸ cells, about 4.5×10⁸ cells, about 5×10⁸ cells, about 5.5×10⁸ cells, about 6×10⁸ cells, about 6.5×10⁸ cells, about 7×10⁸ cells, about 7.5×10⁸ cells, about 8×10⁸ cells, about 8.5×10⁸ cells, about 9×10⁸ cells, about 9.5×10⁸ cells, or about 1×10⁹ cells or more may be used. For subsequent administrations over several weeks or longer, depending on the disease, the treatment is sustained until a desired suppression of symptoms occurs or until sufficient therapeutic levels are achieved, for example, molecular remission.

For example a dosing regimen may comprise administering a first dose of about 0.3×10⁴ cells, about 0.5×10⁴ cells, about 1×10⁴ cells, about 1.5×10⁴ cells, about 2×10⁴ cells, about 2.5×10⁴ cells, about 3×10⁴ cells, about 3.5×10⁴ cells, about 4×10⁴ cells, about 4.5×10⁴ cells, about 5×10⁴ cells, about 5.5×10⁴ cells, about 6×10⁴ cells, about 6.5×10⁴ cells, about 7×10⁴ cells, about 7.5×10⁴ cells, about 8×10⁴ cells, about 8.5×10⁴ cells, about 9×10⁴ cells, about 9.5×10⁴ cells, about 1×10⁵ cells, about 1.5×10⁵ cells, 2×10⁵ cells, about 2.5×10⁵ cells, about 3×10⁵ cells, about 3.5×10⁵ cells, about 4×10⁵ cells, about 4.5×10⁵ cells, about 5×10⁵ cells, about 5.5×10⁵ cells, about 6×10⁵ cells, about 6.5×10⁵ cells, about 7×10⁵ cells, about 7.5×10⁵ cells, about 8×10⁵ cells, about 8.5×10⁵ cells, about 9×10⁵ cells, about 9.5×10⁵ cells, about 1×10⁶ cells, about 1.5×10⁶ cells, 2×10⁶ cells, about 2.5×10⁶ cells, about 3×10⁶ cells, about 3.5×10⁶ cells, about 4×10⁶ cells, about 4.5×10⁶ cells, about 5×10⁶ cells, about 5.5×10⁶ cells, about 6×10⁶ cells, about 6.5×10⁶ cells, about 7×10⁶ cells, about 7.5×10⁶ cells, about 8×10⁶ cells, about 8.5×10⁶ cells, about 9×10⁶ cells, about 9.5×10⁶ cells, about 1×10⁷ cells, about 1.5×10⁷ cells, 2×10⁷ cells, about 2.5×10⁷ cells, about 3×10⁷ cells, about 3.5×10⁷ cells, about 4×10⁷ cells, about 4.5×10⁷ cells, about 5×10⁷ cells, about 5.5×10⁷ cells, about 6×10⁷ cells, about 6.5×10⁷ cells, about 7×10⁷ cells, about 7.5×10⁷ cells, about 8×10⁷ cells, about 8.5×10⁷ cells, about 9×10⁷ cells, about 9.5×10⁷ cells, about 1×10⁸ cells, about 1.5×10⁸ cells, about 2×10⁸ cells, about 2.5×10⁸ cells, about 3×10⁸ cells, about 3.5×10⁸ cells, about 4×10⁸ cells, about 4.5×10⁸ cells, about 5×10⁸ cells, about 5.5×10⁸ cells, about 6×10⁸ cells, about 6.5×10⁸ cells, about 7×10⁸ cells, about 7.5×10⁸ cells, about 8×10⁸ cells, about 8.5×10⁸ cells, about 9×10⁸ cells, about 9.5×10⁸ cells, or about 1×10⁹ cells of allogeneic CAR-T cells.

In some aspects of the methods described herein, a subsequent dose is administered monthly. In some embodiments, a subsequent dose is administered every other month. In some embodiments, a subsequent dose is administered about every three months. In some embodiments, a subsequent dose is administered about every four months. In some embodiments, a subsequent dose is administered about every five months. In some embodiments, a subsequent dose is administered about every six months. In some embodiments, a subsequent dose is administered about every seven months. In some embodiments, a subsequent dose is administered about every eight months. In preferred embodiments, the first dose and the first subsequent and additional subsequent doses are separated in time from each other by at least about four weeks. In some embodiments, a subsequent dose is administered every other month.

In some aspects of the methods described herein, a suitable dosage of the antibody or antigen binding fragment, or a composition of the disclosure to be administered to a subject is in a range of about 0.01 mg/kg of the antibody or antigen binding fragment, or of the composition of the disclosure, to about 100 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, about 20 mg/kg body weight, about 30 mg/kg body weight, about 40 mg/kg body weight, about 50 mg/kg body weight, about 60 mg/kg body weight, about 70 mg/kg body weight, about 80 mg/kg body weight, about 90 mg/kg body weight, or about 100 mg/kg body weight, inclusive of all values and ranges there between.

In some aspects of the methods described herein, a suitable dosage of the antibody or antigen binding fragment, or a composition of the disclosure, to be administered to a subject is in a range of about 0.01 mg/kg of the antibody or antigen binding fragment, or of the composition of the disclosure, to about 100 mg/kg of body weight, in a range of about 0.01 mg/kg to about 90 mg/kg of body weight, in a range of about 0.01 mg/kg to about 80 mg/kg of body weight, in a range of about 0.01 mg/kg to about 70 mg/kg of body weight, in a range of about 0.01 mg/kg to about 60 mg/kg of body weight, in a range of about 0.01 mg/kg to about 50 mg/kg of body weight, in a range of about 0.01 mg/kg to about 40 mg/kg of body weight, in a range of about 0.01 mg/kg to about 30 mg/kg of body weight, in a range of about 0.01 mg/kg to about 20 mg/kg of body weight, in a range of about 0.01 mg/kg to about 10 mg/kg of body weight, in a range of about 0.01 mg/kg to about 9 mg/kg of body weight, in a range of about 0.01 mg/kg to about 8 mg/kg of body weight, in a range of about 0.01 mg/kg to about 7 mg/kg of body weight, in a range of 0.01 mg/kg to about 6 mg/kg of body weight, in a range of about 0.05 mg/kg to about 5 mg/kg of body weight, in a range of about 0.05 mg/kg to about 4 mg/kg of body weight, in a range of about 0.05 mg/kg to about 3 mg/kg of body weight, in a range of about 0.05 mg/kg to about 2 mg/kg of body weight, in a range of about 0.05 mg/kg to about 1.5 mg/kg of body weight, or in a range of about 0.05 mg/kg to about 1 mg/kg of body weight.

In some aspects of the methods described herein, a suitable dosage of the antibody or antigen binding fragment, or a composition of the disclosure, is administered at an amount of from about 0.01 mg of the antibody or antigen binding fragment, or of the composition of the disclosure, to about 100 mg daily, an amount of from about 0.01 mg to about 5,000 mg daily, about 0.01 mg to about 4,000 mg daily, about 0.01 mg to about 3,000 mg daily, about 0.01 mg to about 2,000 mg daily, about 0.01 mg to about 1,000 mg daily, from about 0.01 mg to about 950 mg daily, from about 0.01 mg to about 900 mg daily, from about 0.01 mg to about 850 mg daily, from about 0.01 mg to about 800 mg daily, from about 0.01 mg to about 750 mg daily, from about 0.01 mg to about 700 mg daily, from about 0.01 mg to about 650 mg daily, from about 0.01 mg to about 600 mg daily, from about 0.01 mg to about 550 mg daily, from about 0.01 mg to about 500 mg daily, from about 0.01 mg to about 450 mg daily, from about 0.01 mg to about 400 mg daily, from about 0.01 mg to about 350 mg daily, from about 0.01 mg to about 300 mg daily, from about 0.01 mg to about 250 mg daily, from about 0.01 mg to about 200 mg daily, from about 0.01 mg to about 150 mg daily, from about 0.1 mg to about 100 mg daily, from about 0.1 mg to about 95 mg daily, from about 0.1 mg to about 90 mg daily, from about 0.1 mg to about 85 mg daily, from about 0.1 mg to about 80 mg daily, from about 0.1 mg to about 75 mg daily, from about 0.1 mg to about 70 mg daily, from about 0.1 mg to about 65 mg daily, from about 0.1 mg to about 60 mg daily, from about 0.1 mg to about 55 mg daily, from about 0.1 mg to about 50 mg daily, from about 0.1 mg to about 45 mg daily, from about 0.1 mg to about 40 mg daily, from about 0.1 mg to about 35 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, from about 0.1 mg to about 1 mg daily, or from about 5 mg to about 80 mg daily. In some aspects, the therapeutic composition is administered at a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 2,000 mg, about 3,000 mg, about 4,000 mg, or about 5,000 mg inclusive of all values and ranges there between.

### iV. Kits and Methods of Manufacture

Provided herein are kits comprising one or more chimeric antigen receptors (CAR), antibodies or antigen-binding fragments thereof disclosed herein or conjugates (e.g., detection conjugates) thereof.

In some aspects described herein, a kit of parts comprising at least one of a (i) variable heavy chain, and/or (ii) variable light chain of the (a) the antigen binding molecule comprising a single chain variable fragment (scFv), or (b) the antibody or antigen binding fragment described herein is disclosed.

In some aspects described herein, the kit further comprises instructions for use.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. Such containers may include injection or blow-molded plastic containers into which the desired vials are retained.

When the components of the kit are provided in one and/or more liquid solutions, the liquid solution is an aqueous solution, with a sterile aqueous solution being particularly preferred. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means.

In some aspects described herein, the kit comprises an adenoassociated virus (AAV) vector encoding a chimeric antigen receptor (CAR) comprising (i) an antigen binding molecule that specifically binds to a TCR Vβ region, (ii) an extracellular domain, (iii) a transmembrane domain, (iv) a costimulatory domain and (v) an activating domain, and the antigen binding molecule is a single chain variable fragment (scFv) comprising: a variable heavy chain according to any one of SEQ ID NOs: 45-131 and 534-544, a variable light chain according to any one of SEQ ID NOs: 132-227 and 545-558, or a combination of a variable heavy chain and a variable light chain described herein.

In some aspects, a nucleic acid construct encoding a Vβ region of a TCR-binding CAR is a chimeric nucleic acid molecule comprising different coding sequences, for example, (5′ to 3′) the coding sequences of a human anti-Vβ region of a TCR scFv, a human CD8α-hinge region, a human CD28α transmembrane domain and a CD247 signaling domain. In some aspects, a nucleic acid construct encoding a Vβ region of a TCR-binding CAR is a chimeric nucleic acid molecule comprising different coding sequences, for example, (5′ to 3′) the coding sequences of a human anti-Vβ region of a TCR scFv, a human CD8α-hinge, a human CD28α transmembrane domain, a 4-1BB co-stimulatory domain, and a CD247 signaling domain.

In some aspects described herein, the polynucleotide is, for example, encoding the CAR disclosed, and is inserted into a vector. In some aspects, the vector as used herein is a vehicle into which a polynucleotide encoding a protein can be covalently inserted so as to bring about the expression of that protein and/or the cloning of the polynucleotide. The isolated polynucleotide can be inserted into a vector using any suitable methods known in the art, for example, without limitation, the vector can be digested using appropriate restriction enzymes and then can be ligated with the isolated polynucleotide having matching restriction ends. In some aspects, expression vectors have the ability to incorporate and express heterologous or modified nucleic acid sequences coding for at least part of a gene product capable of being transcribed in a cell. In most cases, RNA molecules are then translated into a protein.

Expression vectors can contain a variety of control sequences, which refer to nucleic acid sequences for the transcription and possibly translation of an operatively linked coding sequence in a particular host organism. In addition to control sequences that govern transcription and translation, vectors and expression vectors can contain nucleic acid sequences that serve other functions as well and are discussed infra. An expression vector can comprise additional elements, for example, the expression vector can have two replication systems, thus allowing it to be maintained in two organisms, for example in human cells for expression and in a prokaryotic host for cloning and amplification.

In some aspects, for expression of a Vβ region of a TCR-binding CAR, the vector can be introduced into a host cell to allow expression of the polypeptide within the host cell. The expression vectors can contain a variety of elements for controlling expression, including without limitation, promoter sequences, transcription initiation sequences, enhancer sequences, selectable markers, and signal sequences. These elements can be selected as appropriate by a person of ordinary skill in the art, as disclosed herein. For example, the promoter sequences can be selected to promote the transcription of the polynucleotide in the vector. Suitable promoter sequences include, without limitation, T7 promoter, T3 promoter, SP6 promoter, beta-actin promoter, EF1a promoter, CMV promoter, and SV40 promoter. Enhancer sequences can be selected to enhance the transcription of the polynucleotide. Selectable markers can be selected to allow selection of the host cells inserted with the vector from those not, for example, the selectable markers can be genes that confer antibiotic resistance. Signal sequences can be selected to allow the expressed polypeptide to be transported outside of the host cell.

For cloning of the polynucleotide, the vector can be introduced into a host cell (an isolated host cell) to allow replication of the vector itself and thereby amplify the copies of the polynucleotide contained therein. The cloning vectors can contain sequence components generally include, without limitation, an origin of replication, promoter sequences, transcription initiation sequences, enhancer sequences, and selectable markers. These elements can be selected as appropriate by a person of ordinary skill in the art. For example, the origin of replication can be selected to promote autonomous replication of the vector in the host cell.

The CARs of the present disclosure can be introduced into a host cell using transfection and/or transduction techniques known in the art. In some aspects, the CAR of the present disclosure is introduced and expressed in immune effector cells so as to redirect their specificity to a target antigen of interest.

The present disclosure provides methods for making the immune effector cells that express the CAR as disclosed herein. In some aspects, the immune effector cells are isolated from an individual and genetically modified without further manipulation in vitro. Such cells can then be directly re-administered into the individual. In some aspects, the immune effector cells are first activated and stimulated to proliferate in vitro prior to being genetically modified to express a CAR. In this regard, the immune effector cells can be cultured before or after being genetically modified (i.e., transduced or transfected to express a CAR as disclosed herein).

Prior to in vitro manipulation or genetic modification of the immune effector cells disclosed herein, the source of cells can be obtained from a subject. In particular, the immune effector cells for use with the CARs as disclosed herein comprise T cells. T cells can be obtained from a number of sources, including peripheral blood mononuclear cells, bone marrow, lymph nodes tissue, cord blood, thymus issue, tissue from a site of infection, ascites, pleural effusion, spleen tissue, and tumors. In some aspects, T cells can be obtained from a unit of blood collected from the subject using any number of techniques known to the skilled person. In some aspects, cells from the circulating blood of an individual are obtained by apheresis. The apheresis product typically contains lymphocytes, including T cells, monocytes, granulocyte, B cells, other nucleated white blood cells, red blood cells, and platelets. In some aspects, the cells collected by apheresis can be washed to remove the plasma fraction and to place the cells in an appropriate buffer or media for subsequent processing. In some aspects, the cells are washed with PBS. In some aspects, the washed solution lacks calcium, and can lack magnesium or can lack many, if not all, divalent cations. As would be appreciated by those of ordinary skill in the art, a washing step can be accomplished by methods known to those in the art, such as by using a semiautomated flowthrough centrifuge. After washing, the cells can be resuspended in a variety of biocompatible buffers or other saline solution with or without buffer. In some aspects, the undesirable components of the apheresis sample can be removed in the cell directly resuspended culture media.

In some aspects, T cells are isolated from peripheral blood mononuclear cells (PBMCs) by lysing the red blood cells and depleting the monocytes, for example, by centrifugation through a PERCOLL™ gradient. A specific subpopulation of T cells, such as CD28+, CD4+, CD8+, CD45RA+, and CD45RO+ T cells, can be further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD1b, CD16, HLA-DR, and CD8. Flow cytometry and cell sorting can also be used to isolate cell populations of interest for use in the present disclosure.

PBMCs can be used directly for genetic modification with the CARs using methods as disclosed herein. In some aspects, after isolation of PBMC, T lymphocytes are further isolated and in some aspects, both cytotoxic and helper T lymphocytes can be sorted into naive, memory, and effector T cell subpopulations either before or after genetic modification and/or expansion. CD8+ cells can be obtained by using standard methods. In some aspects, CD8+ cells are further sorted into naive, central memory, and effector cells by identifying cell surface antigens that are associated with each of those types of CD8+ cells.

In some aspects, memory T cells are present in both CD62L+ and CD62L-subsets of CD8+ peripheral blood lymphocytes. PBMC are sorted into CD62L-CD8+ and CD62L+CD8+ fractions after staining with anti-CD8 and anti-CD62L antibodies. In some aspects, the expression of phenotypic markers of central memory TCM include CD45RO, CD62L, CCR7, CD28, CD3, and CD127 and are negative for granzyme B. In some aspects, central memory T cells are CD45RO+, CD62L+, CD8+ T cells. In some aspects, effector T cells are negative for CD62L, CCR7, CD28, and CD127, and positive for granzyme B and perforin. In some aspects, naive CD8+ T lymphocytes are characterized by the expression of phenotypic markers of naive T cells including CD62L, CCR7, CD28, CD3, CD 127, and CD45RA.

In some aspects, the present disclosure provides isolated host cells containing the vectors provided herein. The host cells containing the vector can be useful in expression or cloning of the polynucleotide contained in the vector. Suitable host cells can include, without limitation, prokaryotic cells, fungal cells, yeast cells, or higher eukaryotic cells such as mammalian cells. Suitable prokaryotic cells for this purpose include, without limitation, eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In some aspects, provided herein is a method of making an antibody or antigen-binding fragment which immunospecifically binds to TCRVβ (e.g., human TCRVβ) comprising culturing a cell or host cell disclosed herein. In some aspects, provided herein is a method of making an antibody or antigen-binding fragment thereof which immunospecifically binds to TCRVβ (e.g., human TCRVβ) comprising expressing (e.g., recombinantly expressing) the antibody or antigen-binding fragment thereof using a cell or host cell disclosed herein (e.g., a cell or a host cell comprising polynucleotides encoding an antibody or antigen-binding fragment thereof disclosed herein). In some aspects, the cell is an isolated cell. In some aspects, the exogenous polynucleotides have been introduced into the cell. In some aspects, the method further comprises the step of purifying the antibody or antigen-binding fragment obtained from the cell or host cell.

Antibodies and antigen-binding fragments thereof that immunospecifically bind to TCRVβ can be produced by any method known in the art for the synthesis of antibodies and antigen-binding fragments thereof, for example, by chemical synthesis or by recombinant expression techniques. The methods disclosed herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are disclosed, for example, in the references cited herein and are fully explained in the literature. See, e.g., Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

Methods for producing polyclonal antibodies are known in the art (see, for example, Chapter 11 in: Short Protocols in Molecular Biology, (2002) 5th Ed., Ausubel F M et al., eds., John Wiley and Sons, New York).

Monoclonal antibodies or antigen-binding fragments thereof can be prepared using a wide variety of techniques known in the art including the use of hybridoma, recombinant, and phage display technologies, yeast-based presentation technologies, or a combination thereof. For example, monoclonal antibodies or antigen-binding fragments thereof can be produced using hybridoma techniques including those known in the art and taught, for example, in Harlow E & Lane D, Antibodies: A Laboratory Manual, (Cold Spring Harbor Laboratory Press, 2nd ed. 1988); Hammerling G J et al., in: Monoclonal Antibodies and T-Cell Hybridomas 563 681 (Elsevier, N.Y., 1981), or as disclosed in Kohler G & Milstein C (1975) Nature 256: 495. Examples of yeast-based presentation methods that can be employed to select and generate the antibodies disclosed herein include those disclosed in, for example, WO2009/036379A2; WO2010/105256; and WO2012/009568, each of which is herein incorporated by reference in its entirety.

Antigen-binding fragments of antibodies disclosed herein can be generated by any technique known to those of skill in the art. For example, Fab and F(ab′)2 fragments disclosed herein can be produced by proteolytic cleavage of immunoglobulin molecules, using enzymes such as papain (to produce Fab fragments) or pepsin (to produce F(ab′)2 fragments). A Fab fragment corresponds to one of the two identical arms of a tetrameric antibody molecule and contains the complete light chain paired with the VH and CH1 domains of the heavy chain. An F(ab′)2 fragment contains the two antigen-binding arms of a tetrameric antibody molecule linked by disulfide bonds in the hinge region.

Further, the antibodies or antigen-binding fragments thereof disclosed herein can also be generated using various phage display and/or yeast-based presentation methods known in the art. In phage display methods, proteins are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scFv linker by PCR and cloned into a phagemid vector. The vector is electroporated in E. coli and the E. coli is infected with helper phage. Phage used in these methods are typically filamentous phage including fd and M13, and the VH and VL domains are usually recombinantly fused to either the phage gene III or gene VIII. Phage expressing an antibody or antigen-binding fragment thereof that binds to a particular antigen can be selected or identified with antigen, e.g., using labeled antigen or antigen bound or captured to a solid surface or bead. Examples of phage display methods that can be used to make the antibodies or fragments disclosed herein include those disclosed in Brinkman U et al., (1995) J Immunol Methods 182: 41-50; Ames R S et al., (1995) J Immunol Methods 184: 177-186; Kettleborough C A et al., (1994) Eur J Immunol 24: 952-958; Persic L et al., (1997) Gene 187: 9-18; Burton D R & Barbas C F (1994) Advan Immunol 57: 191-280; PCT Application No. PCT/GB91/001134; International Publication Nos. WO 90/02809, WO 91/10737, WO 92/01047, WO 92/18619, WO 93/11236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, and 5,969,108.

An antibody or antigen-binding fragment thereof can be selected from any class of immunoglobulins, including IgM, IgG, IgD, IgA, and IgE, and any isotype, including IgG1, IgG2, IgG3 and IgG4.

In some aspects described herein, is a method for engineering/manufacturing a specific target Antibody Dependent Cellular Cytotoxicity (ADCC) activity of a glycosylated and/or afucosylated IgG1 antibody composition comprising: (1) determining the ADCC activity of a glycosylated and afucosylated IgG1 antibody composition; (2) determining a target ADCC activity; and (3) increasing or decreasing the ADCC activity of the glycosylated and afucosylated IgG1 antibody composition by increasing or decreasing the amount of terminal β-galactose in the glycan species at the consensus glycosylation site, wherein the ADCC activity of the glycosylated and afucosylated IgG1 antibody composition after increasing or decreasing the amount of terminal β-galactose is the same as the target ADCC activity or within about 10%, about 15%, about 20%, about 25%, about 30%, about 35%, about 40%, about 45% or about 50% of the target ADCC activity or within about 1% to about 50% of the target ADCC activity.

A variety of host-expression vector systems can be utilized to express antibodies and antigen-binding fragments thereof disclosed herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antibody or antigen-binding fragment thereof disclosed herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7030, HsS78Bst, HeLa, and NIH 3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some aspects, cells for expressing antibodies and antigen-binding fragments thereof disclosed herein are CHO cells, for example CHO cells from the CHO GS System™ (Lonza). In some aspects, cells for expressing antibodies disclosed herein are human cells, e.g., human cell lines. In some aspects, a mammalian expression vector is pOptiVEC™ or pcDNA3.3. In some aspects, bacterial cells such as Escherichia coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells in conjunction with a vector such as the major intermediate early gene promoter element from human cytomegalovirus is an effective expression system for antibodies (Foecking M K & Hofstetter H (1986) Gene 45: 101-10⁵; and Cockett M I et al., (1990) Biotechnology 8: 662-667). In some aspects, antibodies or antigen-binding fragments thereof disclosed herein are produced by CHO cells or NS0 cells.

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells that possess the cellular machinery for proper processing of the primary transcript, glycosylation, and phosphorylation of the gene product can be used. Such mammalian host cells include but are not limited to CHO, VERO, BHK, HeLa, MDCK, HEK 293, NIH 3T3, W138, BT483, Hs578T, HTB2, BT20 and T47D, NS0 (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells. In some aspects, anti-TCRVβ antibodies disclosed herein are produced in mammalian cells, such as CHO cells, e.g., CHO-K1 cells. In some aspects, anti-TCRVβ antibodies disclosed herein are produced in mammalian cells, such as HEK-293 cells.

Once an antibody or antigen-binding fragment thereof disclosed herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies or antigen-binding fragments thereof disclosed herein can be fused to heterologous polypeptide sequences disclosed herein or otherwise known in the art to facilitate purification.

In some aspects, an antibody or antigen-binding fragment thereof disclosed herein is isolated or purified. Generally, an isolated antibody or antigen-binding fragment thereof is one that is substantially free of other antibodies or antigen-binding fragments thereof with different antigenic specificities than the isolated antibody or antigen-binding fragment thereof. For example, in some aspects, a preparation of an antibody or antigen-binding fragment thereof disclosed herein is substantially free of cellular material and/or chemical precursors.

In some aspects, the viral vector containing the coding sequence for the antibody or antigen-binding fragment thereof disclosed herein or the CAR disclosed herein is a retroviral vector or a lentiviral vector.

A lentiviral vector for use herein refers to a vector derived from a lentivirus, a group (or genus) of retroviruses that give rise to slowly developing disease. Viruses included within this group include HIV (human immunodeficiency virus; including HIV type 1, and HIV type 2); visna-maedi; a caprine arthritis-encephalitis virus; equine infectious anemia virus; feline immunodeficiency virus (FIV); bovine immune deficiency virus (BIV); and simian immunodeficiency virus (SIV). Preparation of the recombinant lentivirus can be achieved using the methods according to Dull et al. and Zufferey et al. (Dull et al., J. Virol., 1998; 72: 8463-8471 and Zufferey et al., J. Virol. 1998; 72:9873-9880).

Retroviral vectors (i.e., both lentiviral and non-lentiviral) for use in the present disclosure can be formed using standard cloning techniques by combining the desired DNA sequences in the order and orientation disclosed herein (Current Protocols in Molecular Biology, Ausubel, F. M. et al. (eds.) Greene Publishing Associates, (1989), Sections 9.10-9.14 and other standard laboratory manuals; Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Suitable sources for obtaining retroviral (i.e., both lentiviral and non-lentiviral) sequences for use in forming the vectors include, for example, genomic RNA and cDNAs available from commercially available sources, including the Type Culture Collection (ATCC), Rockville, Md. The sequences also can be synthesized chemically.

### Examples

The following examples are included to demonstrate various aspects of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

### Example 1. CAR-Cells Targeting Vβ2+ T Lymphoma Cells In Vitro

This example shows the expression of CAR-cells targeting T lymphoma cells in an in vitro environment.

mCAR-Vβ2 T-cells were produced from healthy donor CD8+ T-cells, and mouse-anti-human Vβ2 antibody sequences. Antibody targeting human TCR Vβ2 was purchased from Beckman Coulter. Subsequently, high resolution MS was performed by Rapid Novor. Amino acid sequences of the antibody targeting human TCR Vβ2 were then generated by mass spectrometry and were annotated to identify framework and CDR regions of variable domains of heavy and light chains. Peripheral blood (PBMC) from CTCL patients and healthy donors were used to procure CTCL cells, pan-T cells, CD4+ T cells and CD8+ T-cells. Peripheral blood samples were collected in lithium heparin tubes at the Yale Cancer Center in accordance with regulations. The fresh peripheral blood was then 1:1 diluted in room temperature (RT) 1×PBS and layered on Ficol-Plus in SepMate (Stem Cell) centrifuge tubes. The buffy coat of the sample was harvested according to SepMate instructions.

Following extensive PBS washing, the PBMC was then assayed and either directly cryopreserved in liquid nitrogen or immediately used for various purposes, including but not limited to, pan T-cells, CD4 T-cells, CD8 T-cells, or CTCL cell isolation by untouched MACS negative selection kits (Miltenyi).

For CTCL cell isolation, anti-CD26-biotin and/or anti-CD7-biotin were added to remove CD26+ and/or CD7+ cells based on the clinical phenotypes of the malignant CTCL cells in each patient. Isolated T-cell subtypes were either directly cryopreserved in liquid nitrogen or cultured in T-cell medium (RPMI) supplied with 10% heat-inactivated FBS, 20 ng/ml rhIL2, 5 ng/ml rhIL7 and 10 ng/ml rhIL15 (cytokines from R&D Systems).

The four different patient-derived CTCL cells expressing Vβ2 (N=3) or Vβ13.2 (N=1) were cultured overnight with allogeneic mCAR-Vβ2 T-cells at various effector-to-target (E:T, mCAR-Vβ2 effector:CTCL target) ratios of 0, 1:1 and 5:1.

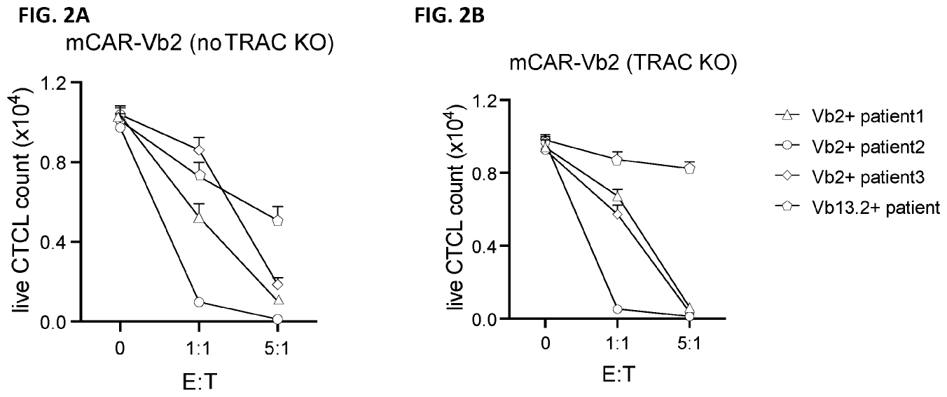

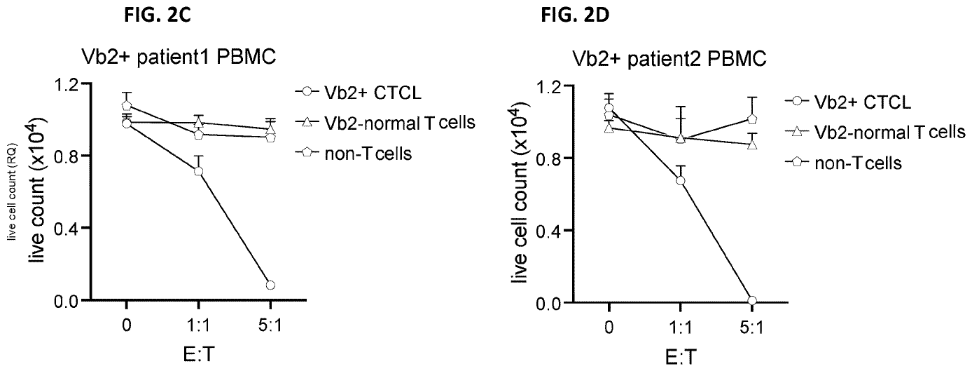

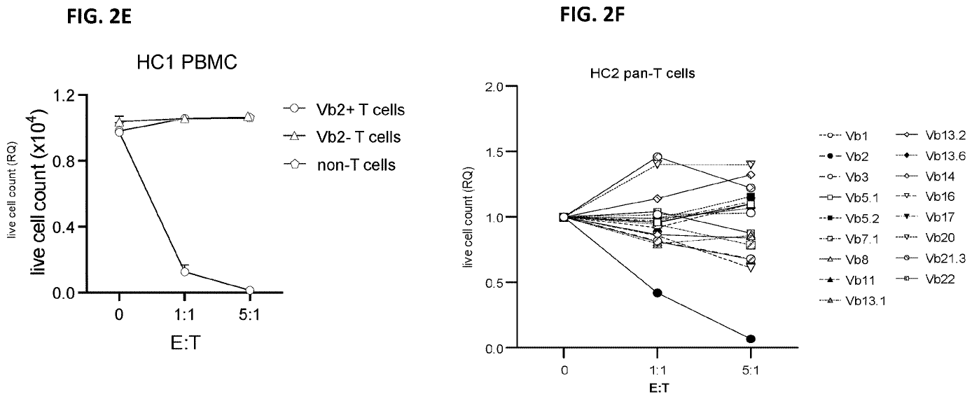

Live CTCL cells were quantified using flow cytometry. Allogeneic mCAR-Vβ2 with TRAC KO from a healthy donor show enhanced specificity and highly effective killing of Vβ2+ CTCL cells as seen in FIG. 2B.

Allogeneic mCAR-Vβ2 from the same healthy donor without gene knockout (KO) of the endogenous T-cell receptor alpha constant (TRAC) region is seen in FIG. 2A, showing the risk of graft-versus-host disease (GVHD) by non-specific killing of Vβ13.2+ cells from a Vβ13.2+ CTCL patient.

Allogeneic mCAR-Vβ2 from the same healthy donor with gene knockout (KO) of the endogenous T-cell receptor alpha constant (TRAC) region is seen in FIG. 2B.

Knock out (KO) was accomplished via sgRNAs targeting human TRAC, B2M and CIITA, which were purchased from IDT. Culture medium was pre-warmed in a 37° C. incubator. Ribonucleoprotein (RNP) complex composed of Cas9 protein and sgRNA was formed at room temperature for 20 minutes. The supernatant was then aspirated completely from the cell pellet. The cell pellet subsequently was then re-suspended in 20 ul P3 buffer (Lonza). Cell suspension, RNP complex, and enhancer (IDT) were mixed thoroughly and then transferred into 16-well strip tubes (Lonza).

Electroporation was performed in Lonza 4D-x unit with program EO-115. Approximately 100 ul of pre-warmed culture medium was immediately added to the cells after electroporation, and then transferred completely into pre-warmed medium for culture.

As shown in FIGS. 2C-2D, allogeneic mCAR-Vβ2 TRAC KO effector T-cells from a healthy donor specifically killed Vβ2+ CTCL cells while sparing Vβ2-negative normal T-cells and non-T-cell. mCAR-Vβ2 TRAC KO effector T-cells were cultured overnight with peripheral blood mononuclear cells (PBMC) from N=2 CTCL patients at varying E:T ratios, then live Vβ2+ CTCL cells, Vβ2-negative normal T-cells, and non-T-cells were quantified by flow cytometry.

Analysis via flow cytometry required suspending cells in a 100 ul 1×PBS containing FcR block (BD) and Aqua Live/Dead fixable dye for 10 minutes at room temperature (RT). Without wash, 100 ul antibody mixture in 1×PBS was added for 30 minutes at 4° C. Following a wash in 1×PBS containing 2% FBS, cells were re-suspended in 200 ul 1×PBS containing 2% FBS and 10 ul counting beads for flow cytometry detection (Thermofisher) were added.

Specificity of allogeneic mCAR-Vβ2 TRAC KO effector T-cells was further assessed following an overnight culture with PBMC, as shown in the healthy control, FIG. 2E, or with purified pan-T-cells (FIG. 2F), by using flow cytometry to quantify either live Vβ2+ T-cells vs total Vβ2-negative T-cells and non-T-cells, or live Vβ2+ T-cells vs T-cells expressing all other identifiable T-cell receptor Vβ families. The relative quantity (RQ) of each large T-cell population count at different E:T ratios was calculated by normalization to the E:T=0 condition. In each experiment N=3 replicates of each effector:target combination were tested.

Endogenous TCR KO, healthy donor-derived allogeneic mCAR-Vβ2 T-cells showed effectiveness and specific in vitro killing ability to Vβ2+ CTCL cells from different patients, and Vβ2+ normal T-cells from different healthy controls, without significant graft versus host disease (GVHD) effects to Vβ2-negative T-cells and non-T-cells.

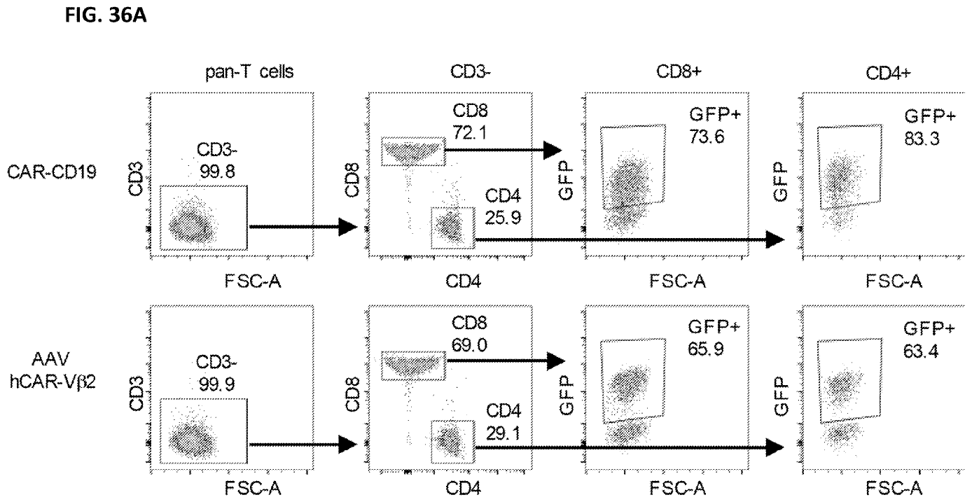

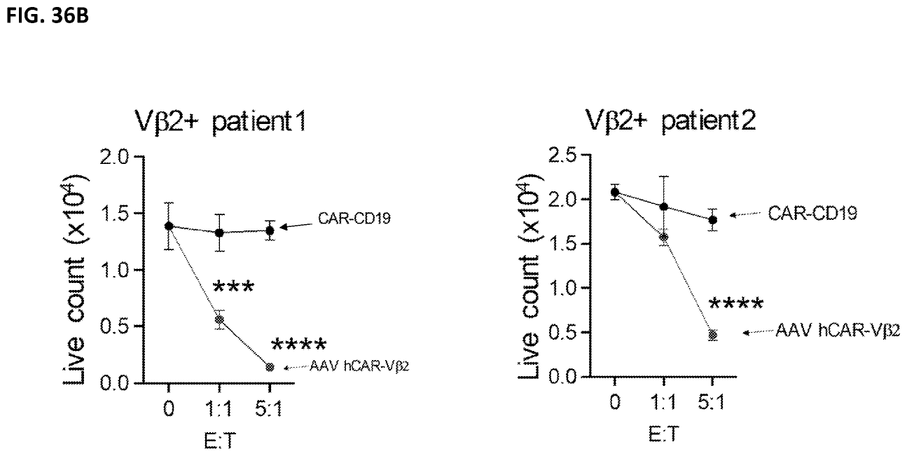

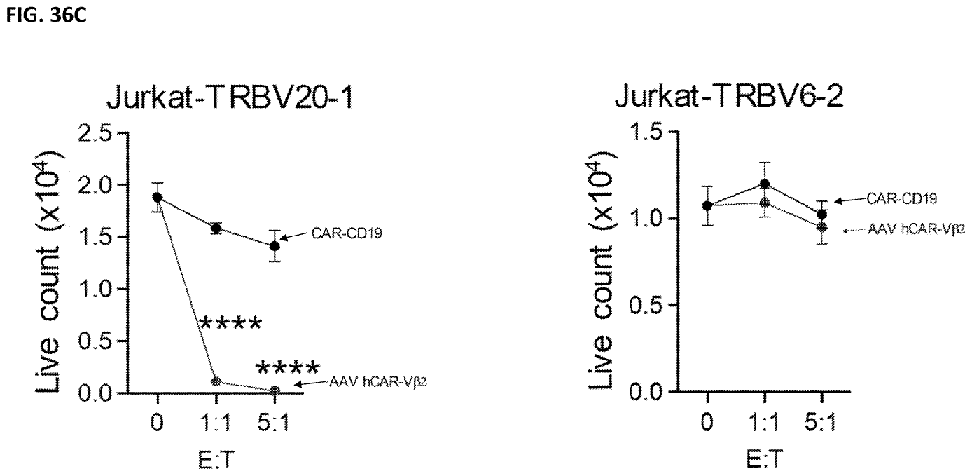

Furthermore the CRISPR-AAV system used for CAR-Vβ2 T cell generation and in vitro killing was illustrated via flow cytometry (see FIGS. 36A-C), which showed CD3− purity, CD4 and CD8 population percentages, and CAR expression of AAV-dependent allogeneic hCAR-Vβ2 T cells compared to lentiviral-dependent allogeneic CAR-CD19 T cells. FIGS. 36B-C shows the live leukemia cell counts from two Vβ2+ CTCL/PTCL patients and live Jurkat-TRBV20-1 (Vβ2+, left) or Jurkat-TRBV6-2 (Vβ13.2+, right) cell counts after an overnight in vitro culture with allogeneic lenti-CAR-CD19 T cells or AAV-hCAR-Vβ2 T cells at different E:T ratios, as determined by flow cytometry described below.

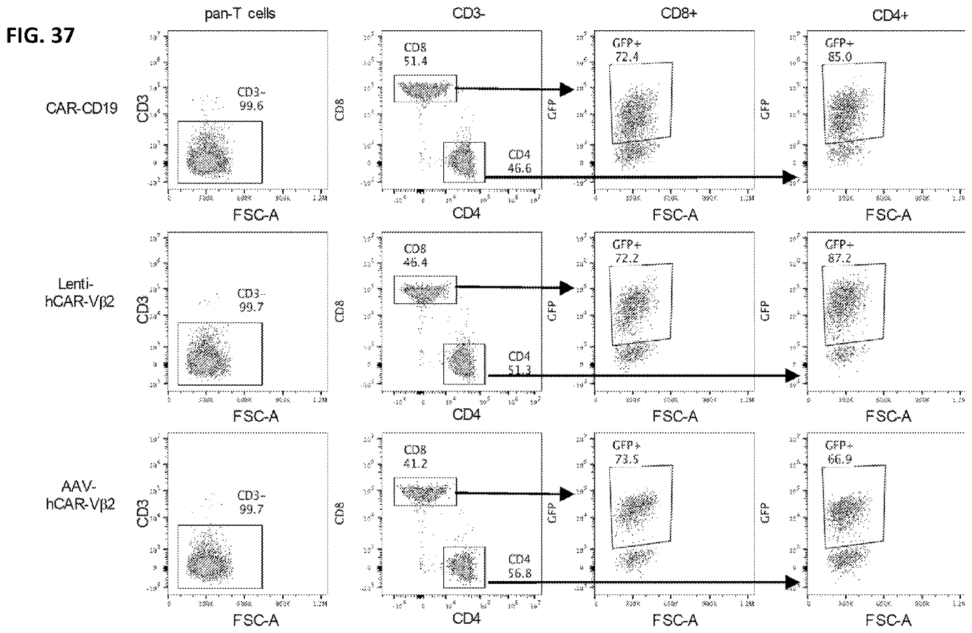

The CRISPR-AAV system for CAR-Vβ2 T cell generation is safer than the lentiviral system because the AAV system directs specific integration of the CAR-construct into donor T cell genomic DNA, and in vitro killing by humanized CAR-Vβ2 T cells made in this way retain high specificity and efficiency as well. FIG. 37 shows CD3− purity, CD4 and CD8 population percentages and expression of green florescence protein (GFP) as a CAR reporter on lenti-CAR-CD19, lenti-hCAR-Vβ2 and AAV-hCAR-Vβ2 T cells, via flow cytometry. Transduction and CAR-expression efficiency of hCAR-Vb2 T cells generated by the CRISPR-AAV system is high, and comparable to the lentiviral system.

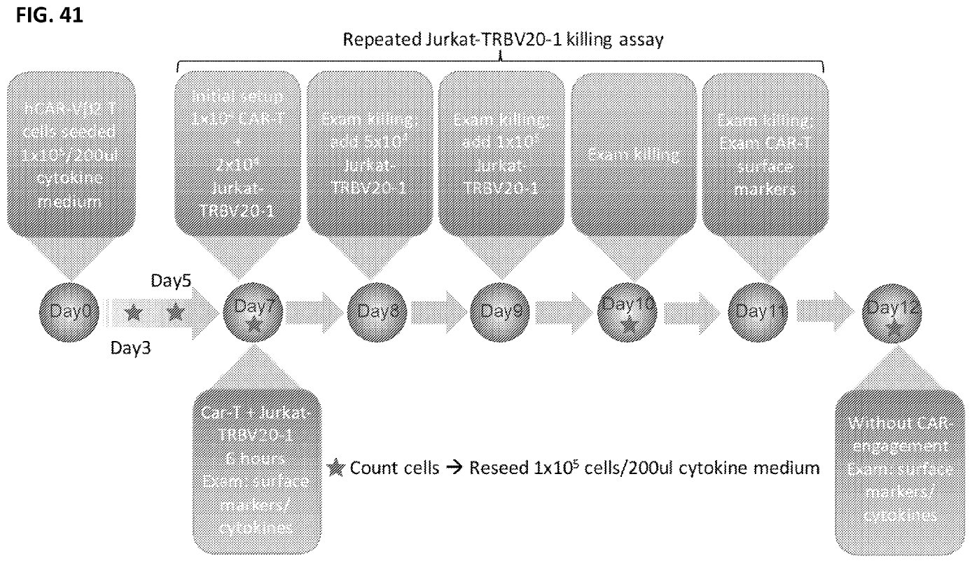

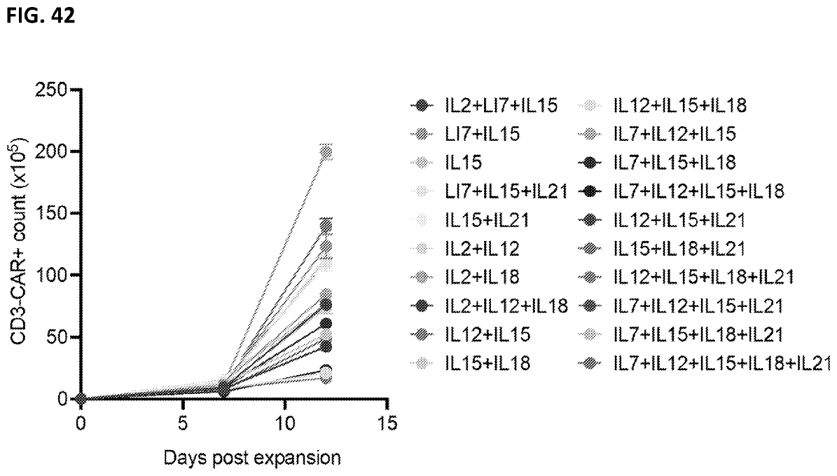

FIG. 41 shows an exemplerary cytokine optimization routine for hCAR-Vβ2 T cell in vitro expansion. The results were compared with one another to identify the optimal culture conditions for hCAR-Vβ2 T cell production with maintainance of high functional capacity, as measured by persistent killing of Vβ2+ malignant T cells, as per FIG. 42. As shown in FIG. 42, live CD3− hCAR-Vβ2 T cell counts at day 0, day 7 and day 12 of expansion under different cytokine combination conditions were studied. Without wishing to be bound by any one theory, but as described herein, cytokine combinations that promote CAR-T cell expansion can be selected from this study as per FIG. 41.

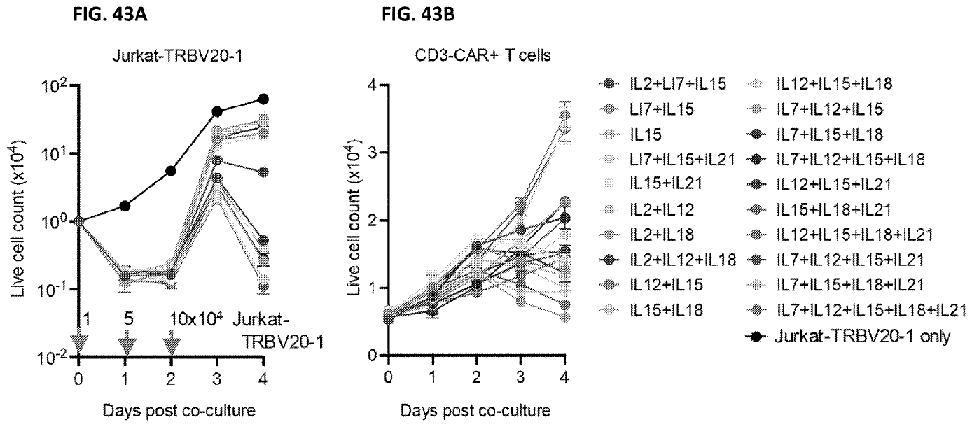

Cytokine optimization for persistent hCAR-Vβ2 T cell killing capacity is evident per FIGS. 43A-43B, as live Jurkat-TRBV20-1 cell counts and live CD3− hCAR-Vβ2 T cell counts from the repeated Jurkat-TRBV20-1 killing assay (see FIG. 41), are determined by trypan blue cell counting and flow cytometry. The cytokine conditions with IL12 and without IL18 enabled persistent malignant T cell killing capacity and improved re-expansion of hCAR-Vβ2 T cells during repeated Jurkat-TRBV20-1 cell killing.

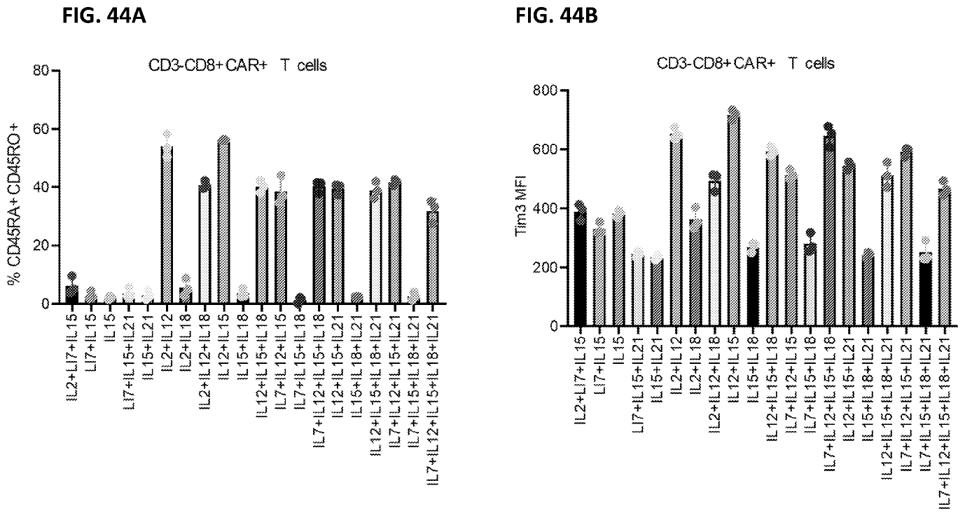

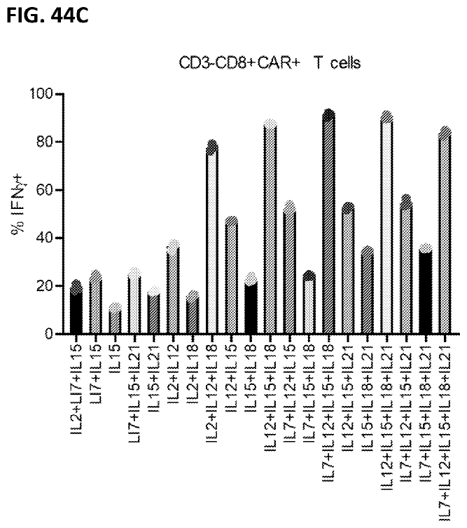

Phenotypic changes in hCAR-Vβ2 T cells post repeated Jurkat-TRBV20-1 killing during cytokine optimization is shown in FIGS. 44A-44C, as CD45RA+CD45RO+% (FIG. 44A) and Tim3 (FIG. 44B) via mean fluorescence intensity (MFI) of CD8+CD3− hCAR-Vβ2 T cells, which were measured 4 days post repeated Jurkat-TRBV20-1 cell killing, as quantified by flow cytometry described below. Furthermore IFNγ+% of 7-day cytokine expanded CD8+CD3− hCAR-Vβ2 T cells after 6-hour Jurkat-TRBV20-1 stimulation in vitro, was quantified by flow cytometry (see FIG. 44C). Cytokine conditions promoting persistent malignant T cell killing effects and better re-expansion of hCAR-Vb2 T cells correlate with phenotypic changes in hCAR-Vb2 T cells post CAR-engagement.

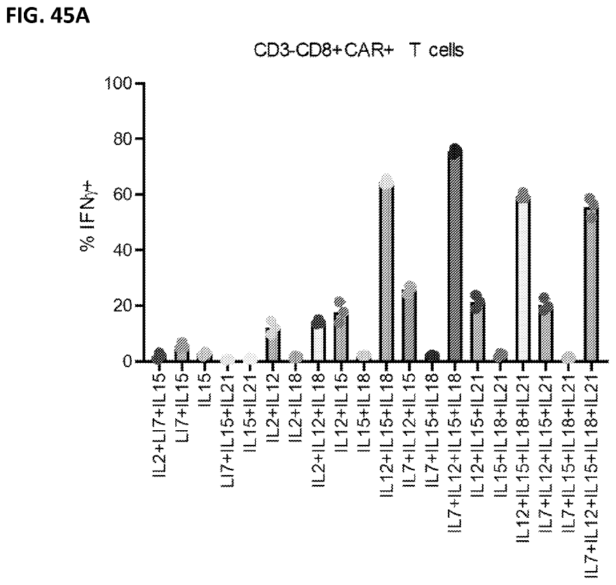

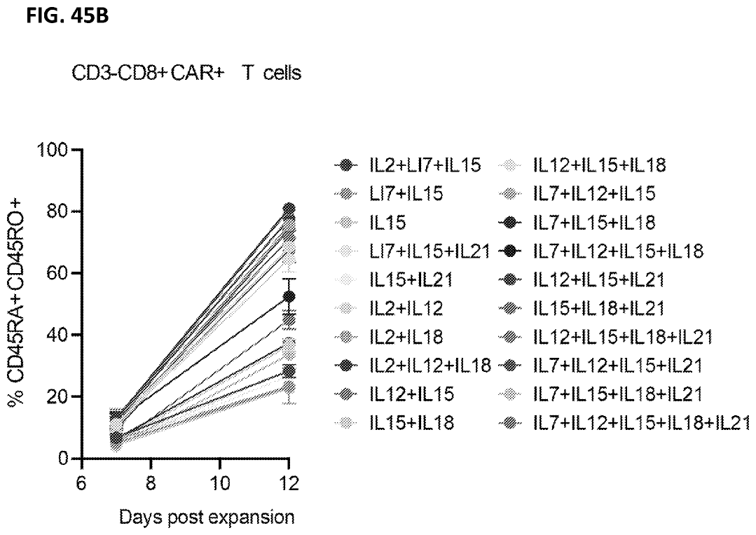

Further phenotypic changes in hCAR-Vβ2 T cells expanded during cytokine optimization are shown as FIGS. 45A-45B. IFNγ+% of 12-day cytokine expanded resting CD8+CD3− hCAR-Vβ2 T cells, as quantified by flow cytometry, is shown as FIG. 45A. CD45RA+CD45RO+% of 7-day and 12-day cytokine expanded resting CD8+CD3− hCAR-Vβ2 T cells, quantified by flow cytometry is shown as FIG. 45B. Cytokine conditions promoting persistent malignant T cell killing and better re-expansion of hCAR-Vβ2 T cells correlated with phenotypic changes in hCAR-Vβ2 T cells during expansion without CAR-engagement.

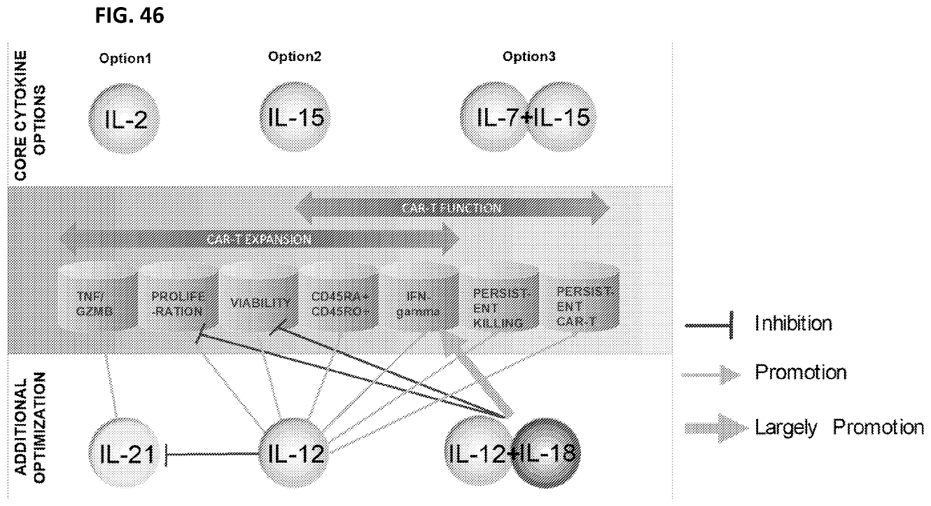

FIG. 46 shows a summary of optimized cytokine combinations for allogeneic hCAR-Vβ2 T cell expansion in vitro. Optimal cytokine combinations promoting CAR-T expansion in vitro, persistent functional capacity and re-expansion of CAR-T during persistent killing of malignant T cells include IL7+IL12+IL15 and IL12+IL15.

### Example 2. CAR Cells Targeting Vβ2+ T Lymphoma Cells In Vivo

This example shows the methodologies of testing CAR-cells targeting T lymphoma cells in an in vivo environment.

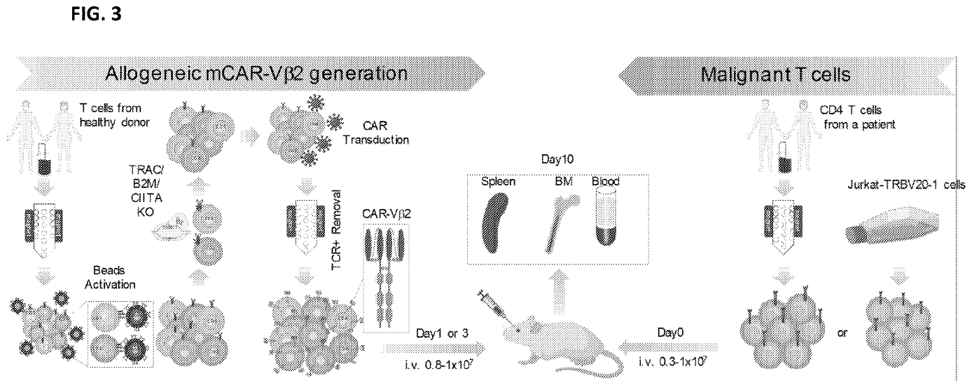

A schematic of the mouse model used to assess in vivo Vβ2+ T lymphoma killing by allogeneic mCAR-Vβ2 T-cells is shown as FIG. 3. mCAR-Vβ2 T-cells with endogenous T-cell receptor knockout (TCR KO) were prepared from in vitro activated healthy donor CD8+ T-cells and the mouse-anti-human Vβ2 antibody sequence. Antibody targeting human TCR Vβ2 was purchased from Beckman Coulter. Subsequently, high resolution MS was performed by Rapid Novor. Amino acid sequences of the antibody targeting human TCR Vβ2 were then generated by mass spectrometry and were annotated to identify framework and CDR regions of variable domains of heavy and light chains.

Immunodeficient NSG mice were then injected intravenously with CD4+ T-cells from a PTCL patient with Vβ2+ malignancy or with a Jurkat T-cell line engineered to express Vβ2. The primary human T-cells freshly isolated or overnight recovered in cytokine-medium after thawing, as disclosed above, were stimulated by anti-CD3/CD28 beads at 1:1 ratio for 2 days. Then beads were magnetically removed and activated T cells were re-suspended in lentiviral supernatant containing cytokines and 8 ug/ml polybrene. Spinfection was then performed for 120 minutes×2000 g at 32° C., followed by changing fresh medium containing cytokines. After overnight culture, T cells were centrifuged and re-suspended in fresh medium containing cytokines for further expansion.

Jurkat T-cells were re-suspended in lentiviral supernatant with 8 ug/ml polybrene. Spinfection was then performed 90 minutes×1200 g at 32° C., followed by changing fresh medium.

Three days following the initial injection, half of each group of mice received allogeneic mCAR-Vβ2 effector T-cells intravenously. Following one week, all mice were euthanized and analyzed. Flow cytometry was utilized to quantify various cell populations in the spleen, bone marrow (BM) and blood of the analyzed mice.

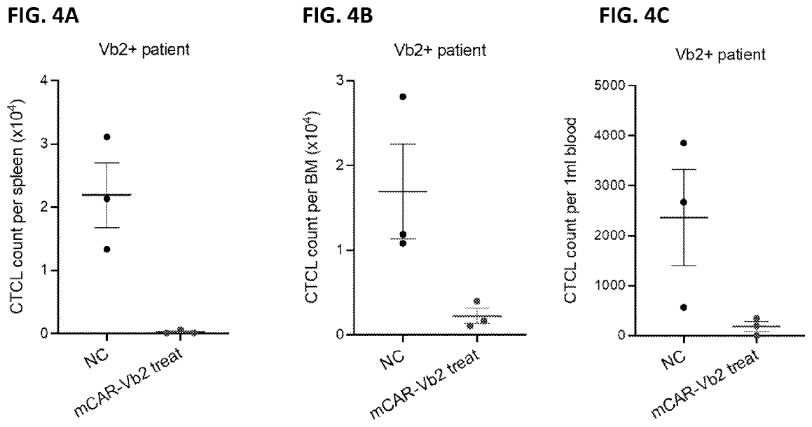

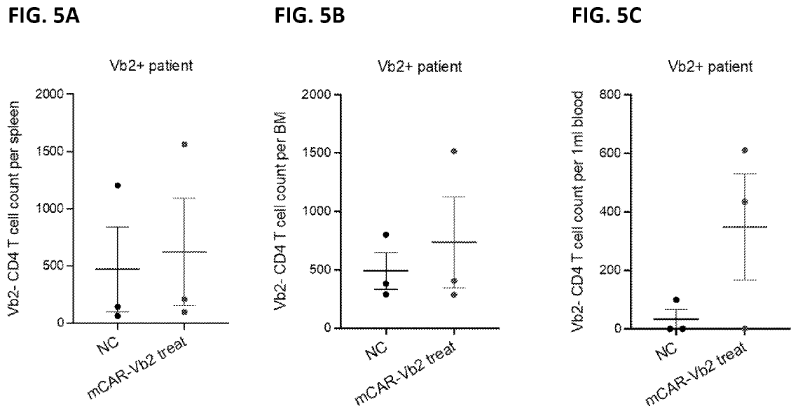

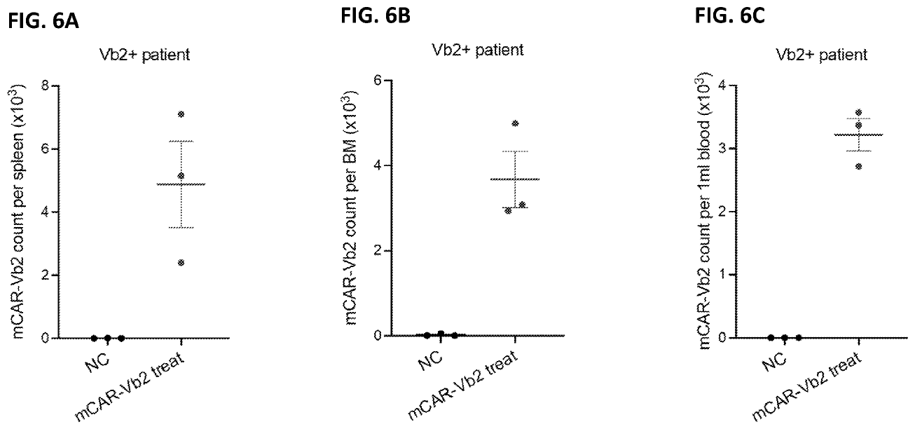

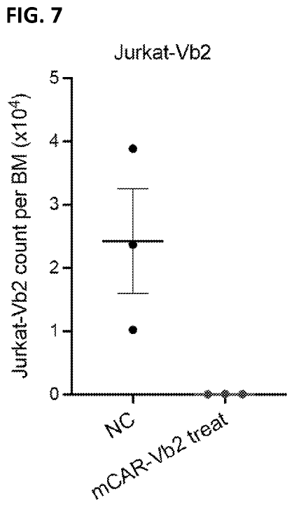

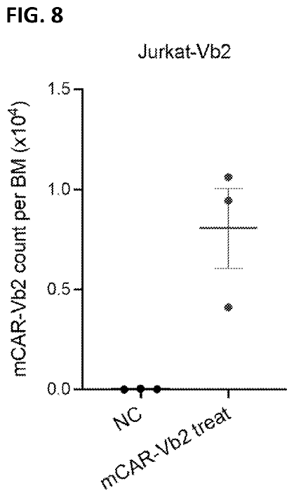

FIGS. 4A-4C illustrate the Vβ2+ PTCL count per spleen (FIG. 4A), bone marrow (FIG. 4B), and blood samples (FIG. 4C). FIGS. 5A-5C illustrate the Vβ2-negative CD4 T-cell count per spleen (FIG. 5A), bone marrow (FIG. 5B), and blood samples (FIG. 5C). FIGS. 6A-6C illustrate the mCAR-Vβ2 count per spleen (FIG. 6A), bone marrow (FIG. 6B), and blood samples (FIG. 6C). FIG. 7. shows the Vβ2+ Jurkat T-cell count per bone marrow sample. FIG. 8. shows the mCAR-Vβ2 T-cell count per bone marrow of NSG mice following 10 days of inoculation of the Jurkat-Vβ2 cells.

Healthy donor-derived TCR-KO mCAR-Vβ2 T-cells showed not only effective and specific in vivo killing ability to Vβ2+ PTCL cells from a patient and to a Vβ2+T lymphoma cell line, but also in vivo persistence for a week post adoptive transfer, suggesting the high efficacy and safety of off-the-shelf allogeneic CAR-cells targeting monoclonal TCR-Vβ2+ T lymphoma in vivo.

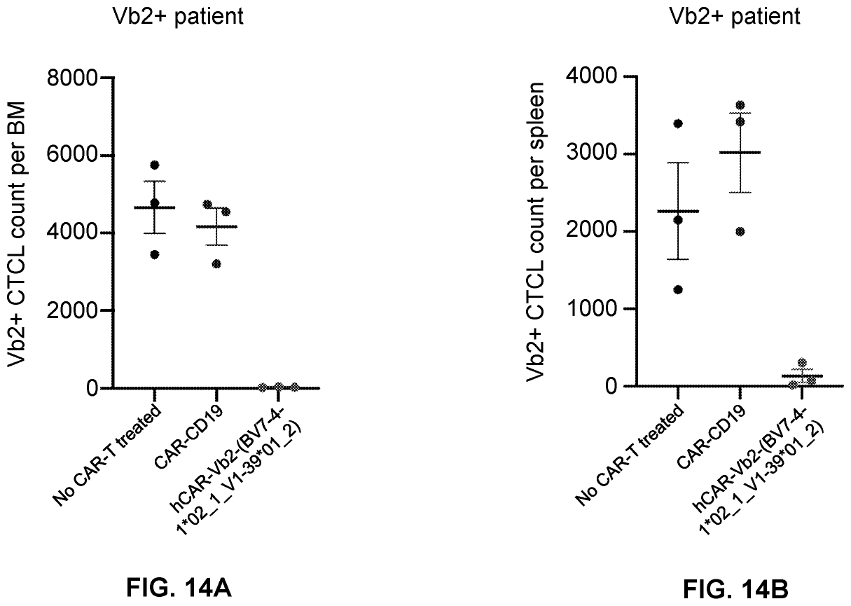

FIGS. 14A-14B illustrate the Vβ2+ CTCL count per bone marrow (FIG. 14A) and per spleen (FIG. 14B) samples, between no CAR treated, CAR-CD19, and hCAR-Vβ2-(BV7-4-1*02_1_V1-39*01_2) in NSG mice populated with pan-CD4+ T cells from a Vβ2+ CTCL patient, as described above. These samples were measured in NSG mice 4 days after the initial inoculation of pan-CD4 T cells from a Vβ2+ patient with three days allogeneic triple-KO hCAR-Vb2 or CAR-CD19 pan-T cell treatment, generated from a healthy donor compared to no CAR cell treatment control as described above.

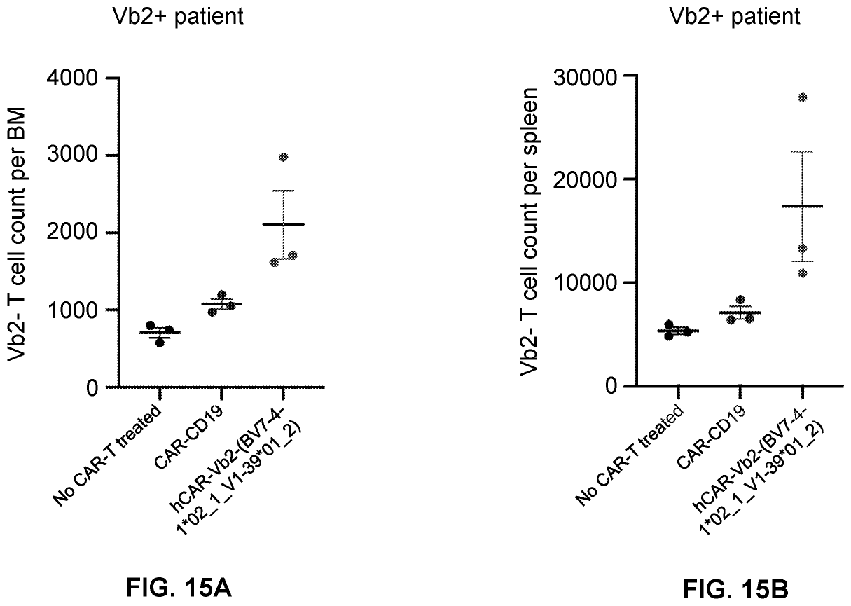

FIGS. 15A-15B illustrate the normal T cell counts per bone marrow (FIG. 15A) and per spleen (FIG. 15B) samples, between no CAR treated, CAR-CD19, and hCAR-Vβ2− (BV7-4-1*02_1_V1-39*01_2) in NSG mice populated with pan-CD4+ T cells from a Vβ2+ CTCL patient from the methods described above. These samples were measured in NSG mice 4 days after the initial inoculation of pan-CD4 T cells from a Vβ2+ patient with three days allogeneic triple-KO hCAR-Vb2 or CAR-CD19 pan-T cell treatment, generated from a healthy donor compared to no CAR cell treatment control methods described above. The hCAR-Vβ2 treatment is highly specific and non-toxic in eliminated Vβ2+ CTCL cells (FIGS. 14A-14B) while not eliminating the Vb2− T cells, as shown in FIGS. 15A-15B.

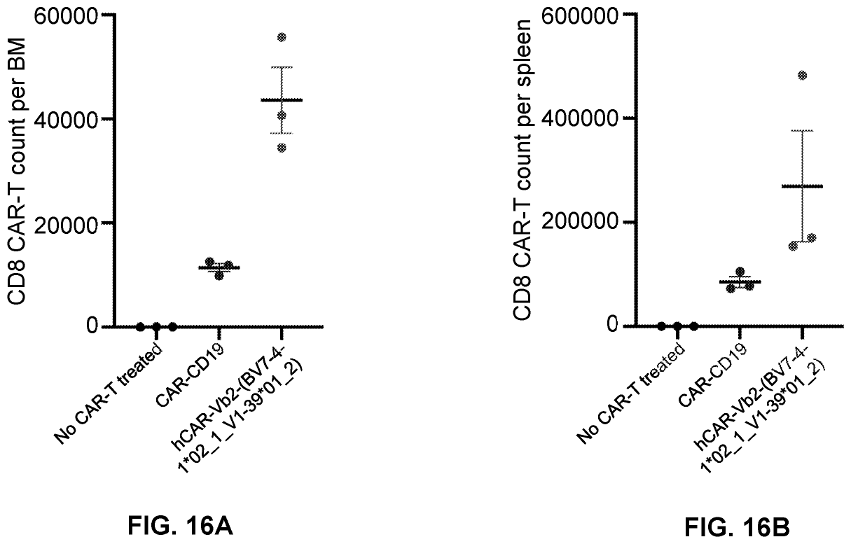

FIGS. 16A-16B illustrate the persistence and expansion of hCAR-Vβ2 CD8+ T cells per bone marrow (FIG. 16A) and per spleen samples (FIG. 16B), between no CAR treated, CAR-CD19, and hCAR-Vβ2-(BV7-4-1*02_1_V1-39*01_2) treatments in NSG mice populated with pan-CD4+ T cells from a Vβ2+ CTCL patient from the methods described above. These samples were measured in NSG mice 4 days after the initial inoculation of pan-CD4 T cells from a Vβ2+ patient with three days allogeneic triple-KO hCAR-Vb2 or CAR-CD19 pan-T cell treatment, generated from a healthy donor and compared to a no CAR cell treatment control from the methods described above. The hCAR-Vβ2 treatment is highly specific and non-toxic in eliminated Vβ2+ CTCL cells (FIGS. 14A-14B and FIGS. 15A-15B, respectively), while persisting and expanding.

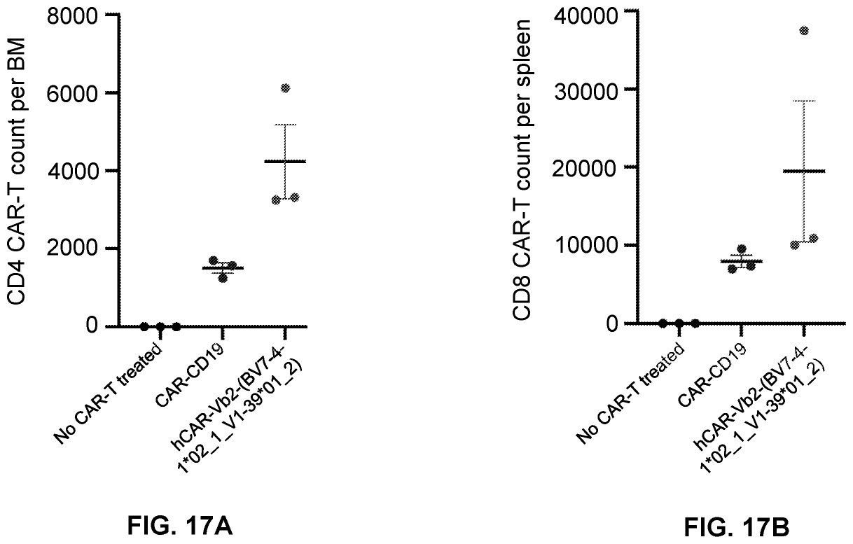

FIGS. 17A-17B illustrate the persistence and expansion of humanized hCAR-Vβ2 CD4+ T cells per bone marrow (FIG. 17A) and per spleen samples (FIG. 17B), between no CAR treated, CAR-CD19, and hCAR-Vβ2-(BV7-4-1*02_1_V1-39*01_2) treatments in NSG mice populated with pan-CD4+ T cells from a Vβ2+ CTCL patient from the methods described above. These samples were measured in NSG mice 4 days after the initial inoculation of pan-CD4 T cells from a Vβ2+ patient with three days allogeneic triple-KO hCAR-Vb2 or CAR-CD19 pan-T cell treatment, generated from a healthy donor compared to a no CAR cell treatment control from the methods described above. The hCAR-Vβ2 treatment is highly specific and non-toxic in eliminated Vβ2+ CTCL cells (FIGS. 14A-14B), while persisting and expanding.

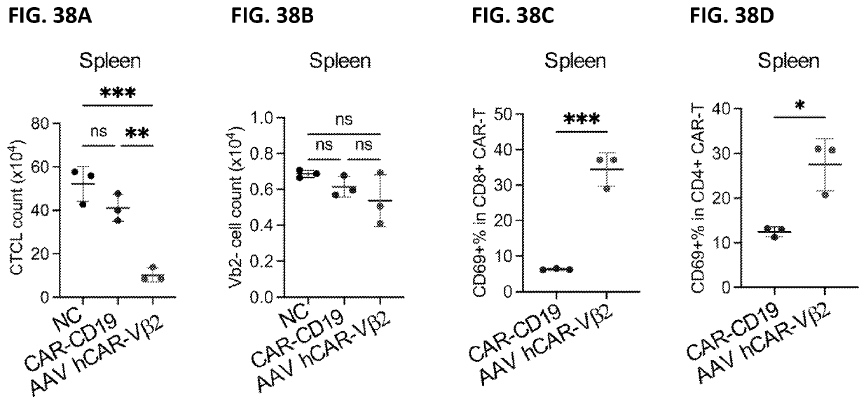

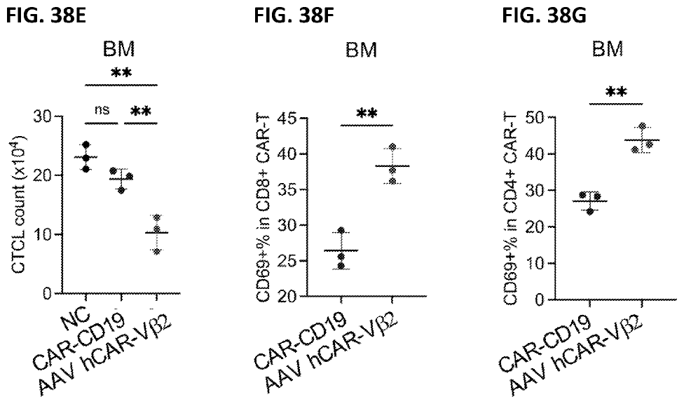

FIGS. 38A-38G show the total CD4 T cells isolated from a Vβ2+ PTCL patient that were adoptively transferred into groups of NSG mice, which were then treated with allogeneic triple-KO AAV-hCAR-Vβ2 or lenti-CAR-CD19 cells generated from healthy donor pan T cells, and then compared to no-treatment control (NC).

Following three days post-treatment Vβ2+ CTCL cells (FIG. 38A), Vβ2− normal T cells (FIG. 38B), CD69+% in CD8 CAR-T cells (FIG. 38C), and CD69+% in CD4 CAR-T cells in spleen (FIG. 38D) were quantified by flow cytometry, as were Vβ2+ CTCL cells (FIG. 38E), CD69+% in CD8 CAR-T cells (FIG. 38F), and CD69+% in CD4 CAR-T cells in bone marrow (BM) (FIG. 38G). As shown in FIGS. 38A-38G, in vivo killing by humanized CAR-Vβ2 T cells generated by the CRISPR-AAV system is highly specific and efficient as well.

### Example 3. Humanized Allogeneic CAR-Cells Targeting Vβ2+ T Lymphoma Cells In Vitro

This example shows the effectiveness of humanized allogeneic CAR-Vβ2 T-cells.

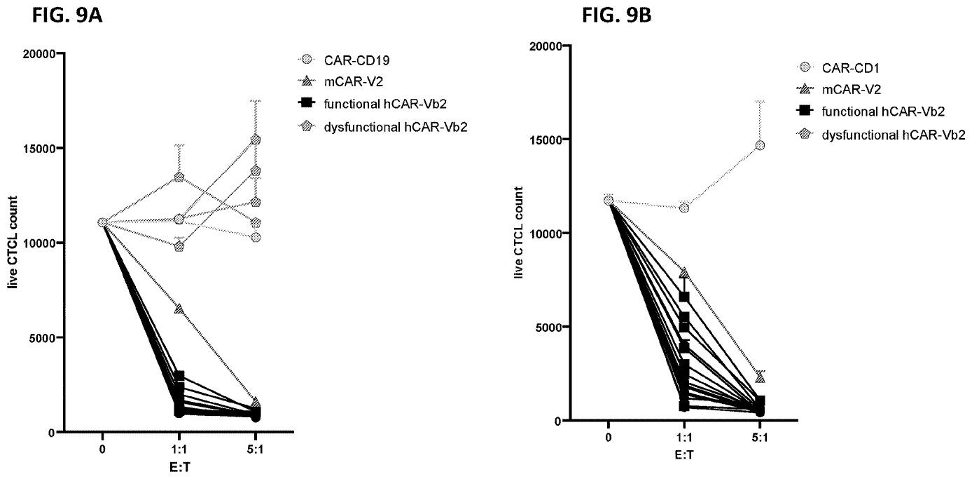

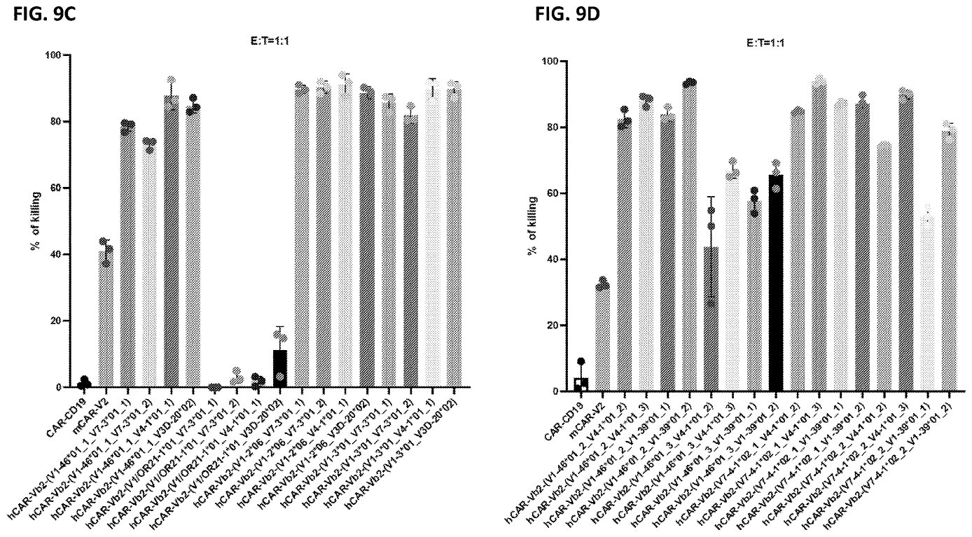

Two different in silico strategies were utilized to produce humanized anti-Vβ2 antibody sequences. BioPhi algorithm or a third-party contractor (mAbvice) may be used. Humanized hCAR-Vβ2 T-cells were produced from healthy donor CD8+ T-cells and were compared with the original mouse mCAR-Vβ2, or control CAR-CD19 for the ability to kill Vβ2+ CTCL cells as shown in FIGS. 9A-9B.

Following an overnight co-culture at multiple effector CAR to Vβ2+ CTCL target (E:T) ratios, flow cytometry, as described in Example 1, was then used to quantify live V2+ CTCL cells. Both humanization strategies generated multiple candidates that killed Vβ2+ CTCL cells more efficiently than the original mouse mCAR-Vβ2 as shown in FIGS. 9A-9D.

Humanization of the mouse version scFv domain enhanced Vβ2+ CTCL killing effects, suggesting the advantages of CAR humanization by in silico strategy.

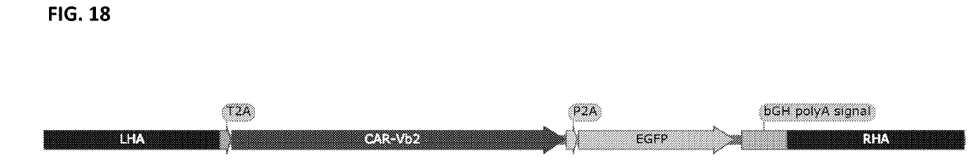

FIG. 18 illustrates the Adeno-associated virus (AAV) chimeric antigen-receptor (CAR) vector plasmid structure. The AAV vector plasmid is comprised of a left homology arm (LHA) of human TRAC region, T2A in-frame to TRAC coding sequence, CAR-V2, a P2A peptide, EGFP as a CAR expression reporter, bGH poly A signal for transcription stop, right homology arm (RHA) of human TRAC region.

The mCAR-Vβ2 expressing AAV vector used a pAAV.CMV.PI.EGFP.WPRE.bGH plasmid (Addgene #105530). The left and right homology arms of the human TRAC region and CAR-GFP fragment, as shown in FIG. 18, were generated via PCR using gDNA from T cells of a healthy donor and CAR-containing lentiviral plasmid as templates, respectively. The WPRE structure from the AAV backbone was then removed, followed by the sequential insertion of (1) left homology arm, (2) T2A, (3) CAR, (4) a P2A peptide, (5) GFP, and (6) right homology arm to generate the CAR-expressing vector as pAAV.pTRA.T2A.CAR.P2A.GFP.bGH. The pSLCAR-CD19-BBz (#135992), pAW13.lentiguide.mcherry (#104375), pMD2.G (#12259) and psPAX2 (#12260) plasmids were purchased from Addgene.

To produce the AAV, AAV transfer plasmid and pDGM6 were co-transfected into 293T cells that were harvested three days later. AAV in transfected 293T cells were then extracted and purified by AAVpro purification kit (TaKaRa, Cat. 6675). The respective virus titer was then measured by qPCR.

To generate CAR-expressing T cells generated by the AAV vector, T cells isolated from a healthy donor were activated by anti-CD3/CD28 beads at 1:1 ratio for about two days, and beads were removed followed by RNP-dependent TRAC/B2M/CIITA triple KO. Following a 1 hour rest in complete T cell culture medium, T cells were resuspended in serum-free T cell culture medium containing cytokines and AAV (MOI=2×10⁴) at 1×10⁷ cells/ml concentration and then incubated at 37° C. for 30 minutes. Complete T cell culture mediums containing 10% FBS, cytokines and homology recombination enhancer cocktail (M3814/TSA/XL413) were then added to a cell concentration of 1×10⁶/ml. Homology recombination enhancer cocktail was then removed following an overnight incubation period. The one day culture was then replaced with a fresh complete T cell medium. Remaining CD3+ cells were then removed as described above for the two to three days post KO methodology.

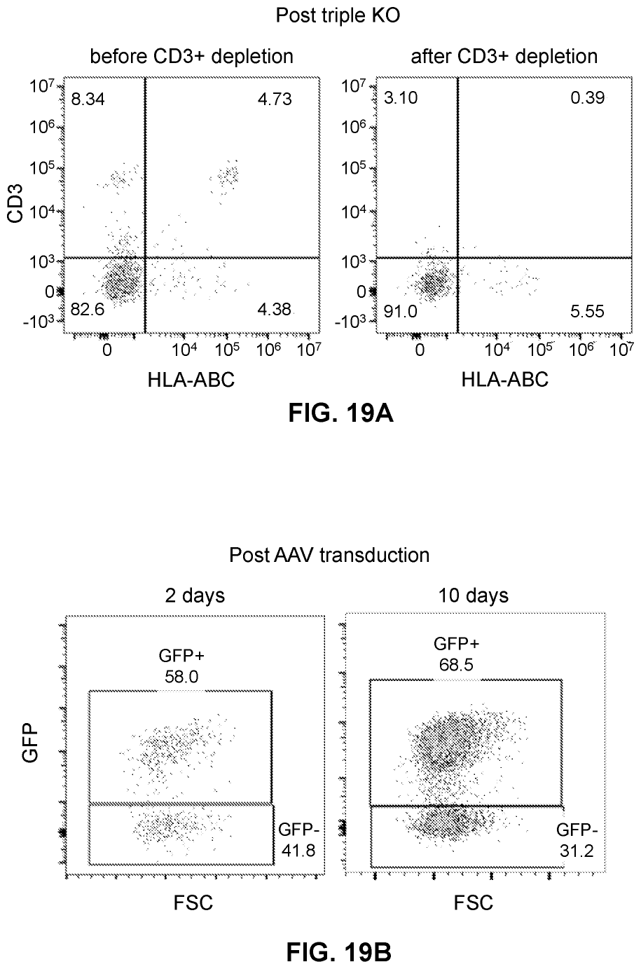

FIG. 19A illustrates CAR generation via TRAC/B2M/CIITA triple-KO and AAV-CAR delivery of CD3, HLA-A/B/C on pan-T cells before and after CD3+ cell depletion using CD3+ microbeads from the methods above. Two days post TRAC/B2M/CIITA triple KO and AAV-CAR-Vb2 transduction, the expression of CD3, HLA-A/B/C on pan-T cells were detected by flow cytometry before and after CD3+ cell depletion using CD3+ microbeads.

FIG. 19B illustrates the effectiveness of post TRAC/B2M/CIITA triple KO and AAV-CAR-Vβ2 transduction. Additionally, the expression of GFP as CAR expressing reporter on CD3-HLA-A/B/C-T-cells, two (left) and ten (right) days post transduction is shown. Two-day and ten-day post TRAC/B2M/CIITA triple KO and AAV-CAR-Vb2 transduction and expression of GFP as a CAR expressing reporter on CD3-HLA-A/B/C-T cells, was then detected by flow cytometry from the methods of example 1.

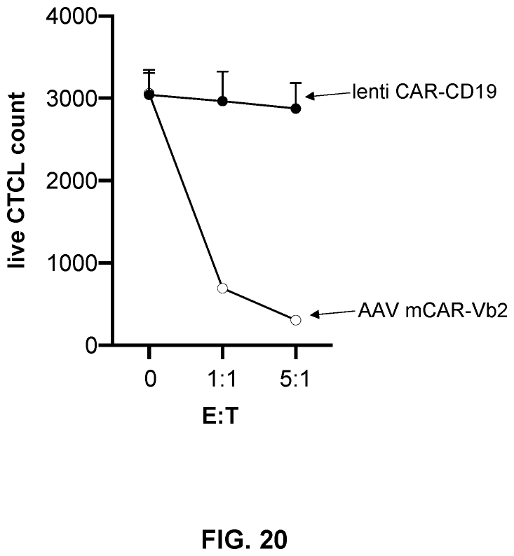

FIG. 20 illustrates the effectiveness of AAV mCAR-Vβ2 via a TRAC/B2M/CIITA triple-KO and AAV-CAR strategy, in Vβ2+ CTCL killing as opposed to a lentiviral delivered CAR-CD19 method. In vitro killing was determined at different effector to target ratios from CTCL cells with a Vβ2+ CTCL patient via flow cytometry as disclosed above.

### Example 4. CAR-Cells Targeting Vβ2+ T Lymphoma Cells

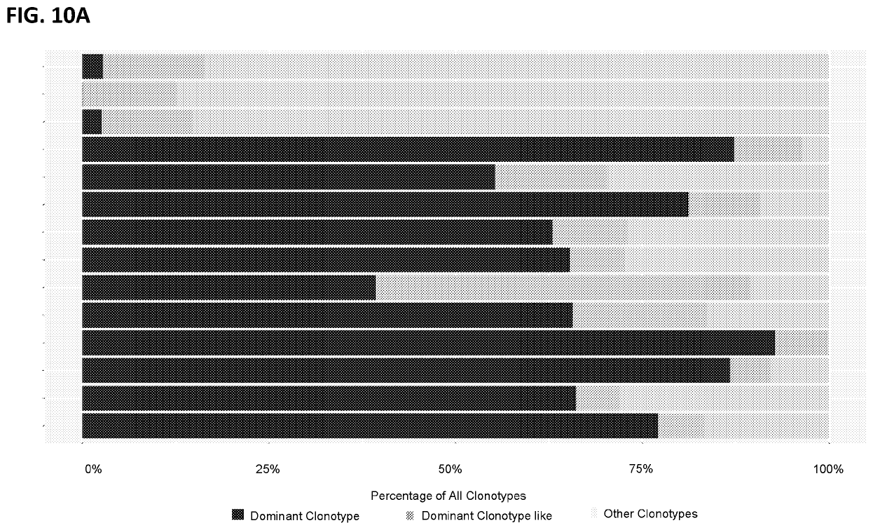

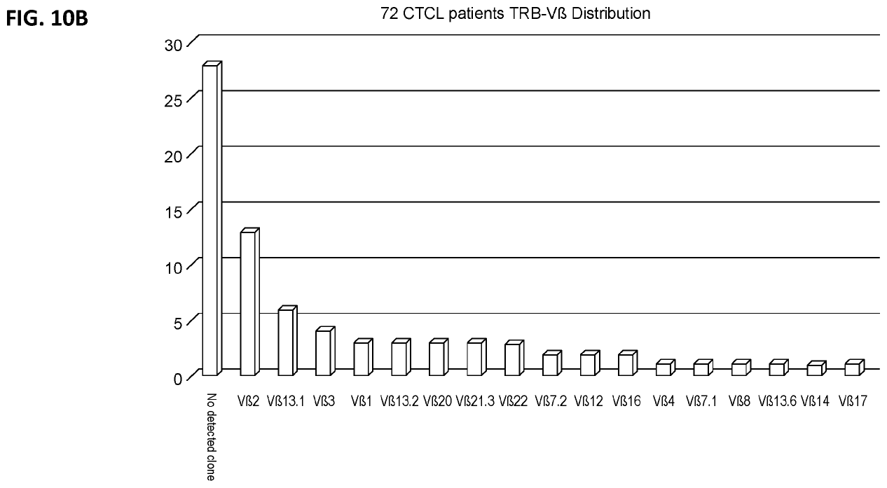

Single cell RNA sequencing was combined with T-cell receptor (TCR) sequencing to identify and quantify T-cell clones in the peripheral blood of 11 CTCL patients with Sezary syndrome (P1 to P11) and 3 healthy controls (N1 to N3) as shown in FIG. 10A. The monoclonal nature of CTCL, evidenced by the presence of a massively expanded clone in each patient, is compared to the polyclonality of T-cells in each healthy control.

TCR Vβ usage frequency distribution in CTCL cells from 72 patients was then determined by anti-Vβ antibody staining and flow cytometry and charted as seen in FIG. 10B. Vβ2+ CTCL occurred most frequently. The “No clone detected” column represents cases where no antibody is currently available.

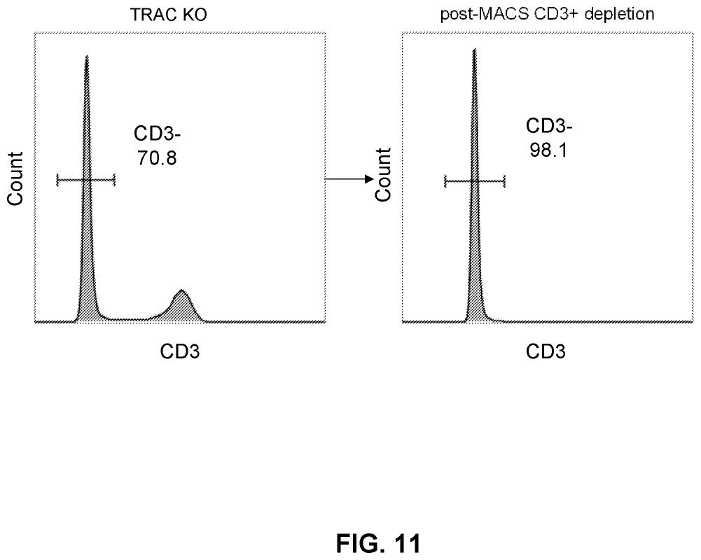

Flow cytometry showing efficiency of T-cell receptor alpha constant region knock out (TRAC KO) was identified as 70.8% in mCAR-Vβ2 T-cells, (left side of FIG. 11), and purity of TRAC KO mCAR-Vβ2 T-cells after magnetic bead removal of remaining CD3+ cells is >98% (right side of FIG. 11).

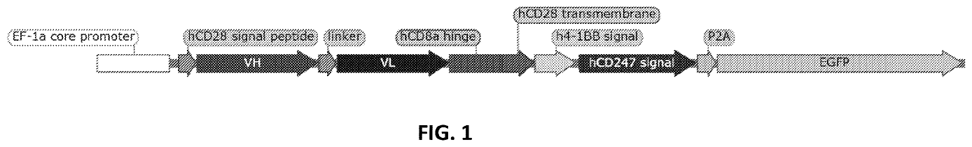

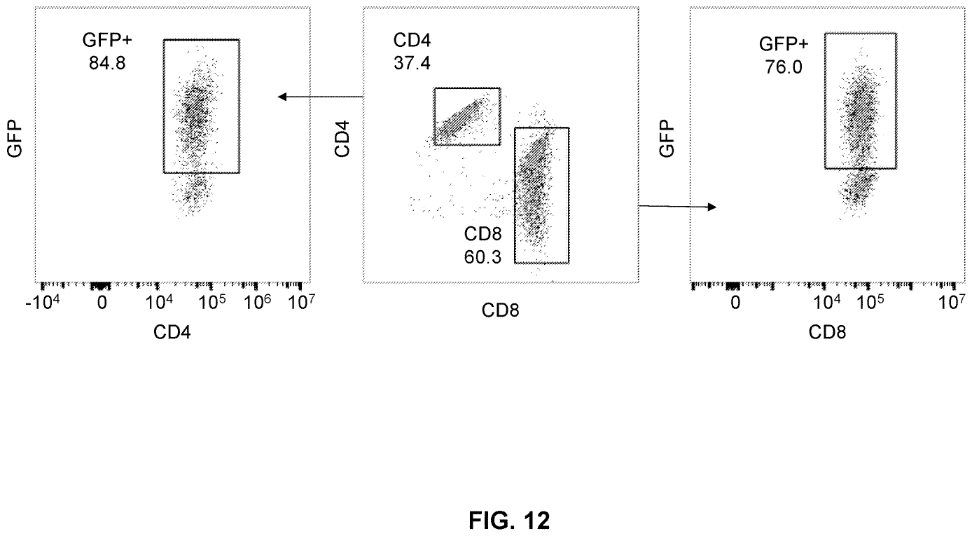

Flow cytometry exhibits the proportions of CD4+ and CD8+ T-cells during CAR-cell in vitro expansion (center of FIG. 12) and the lentiviral transduction efficiency in each subtype (left CD4+, right CD8+) was determined by green florescence (GFP) expression. The CAR-Vβ2 construct includes enhanced GFP (EGFP, as seen in FIG. 1) as a marker of transduction.

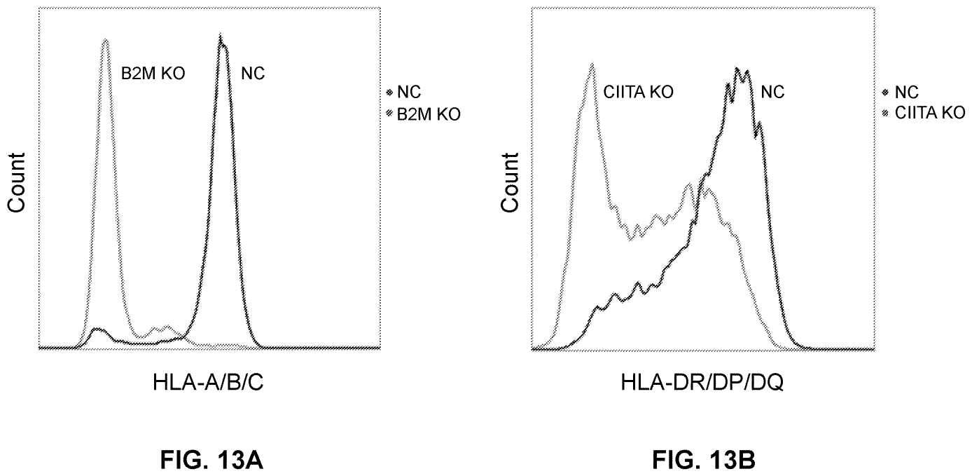

Flow cytometry was then used to show reduction in MHC class I protein expression by mCAR Vβ2 T-cells following knock out of beta-2-microglobulin (B2M KO) compared with a no knock-out control (NC) as seen in FIG. 13A. Flow cytometry showing reduction in MHC class II protein expression by mCAR-Vβ2 T-cells following knock out of the class II major histocompatibility complex transactivator (CIITA KO) compared with a no knock-out control (NC) is seen in FIG. 13B.

### Example 5. Immunogenicity Assay and In Vivo Imaging

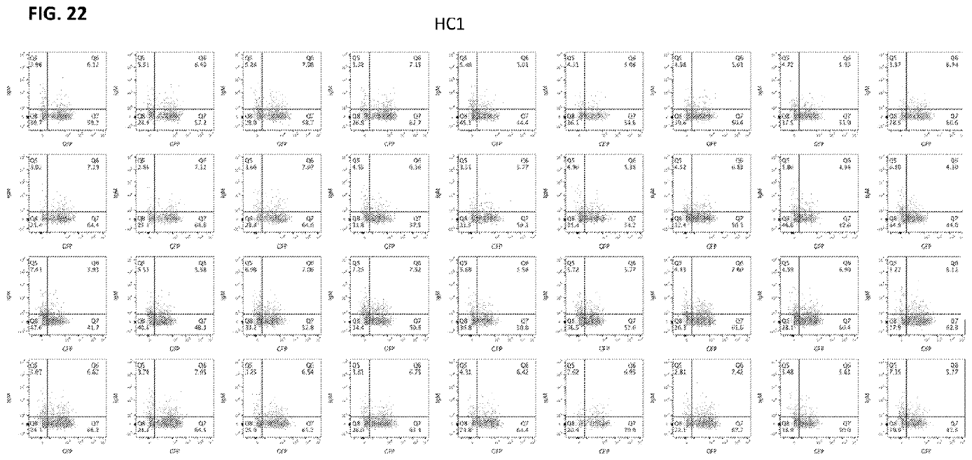

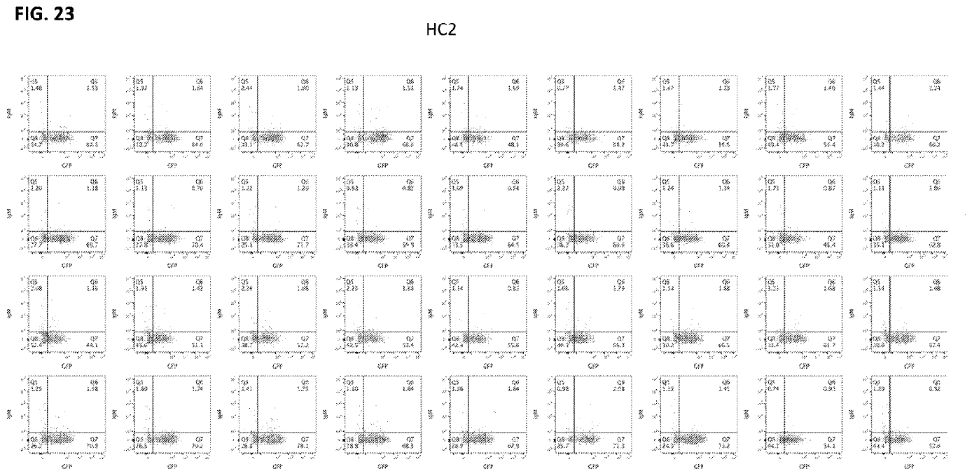

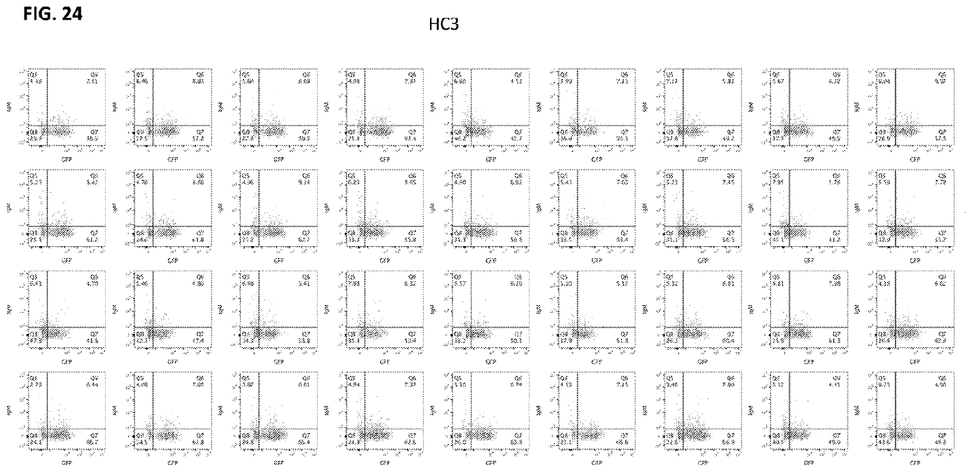

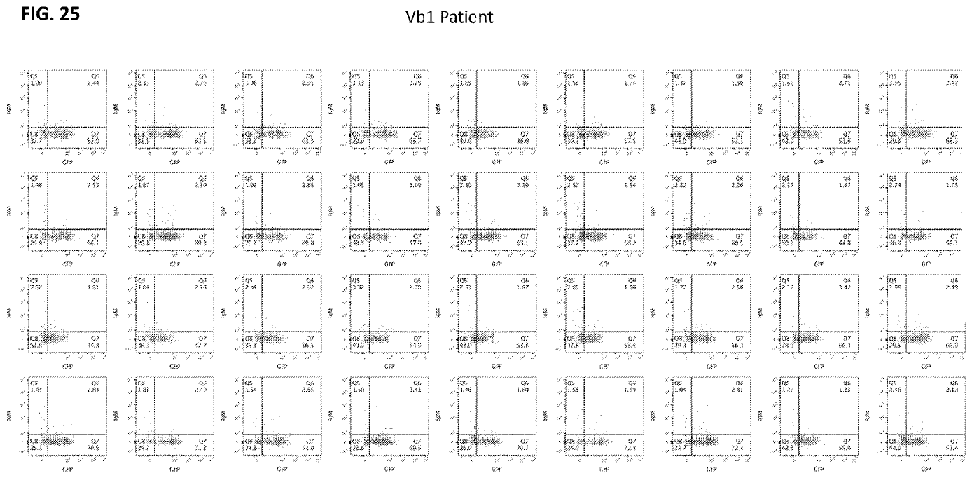

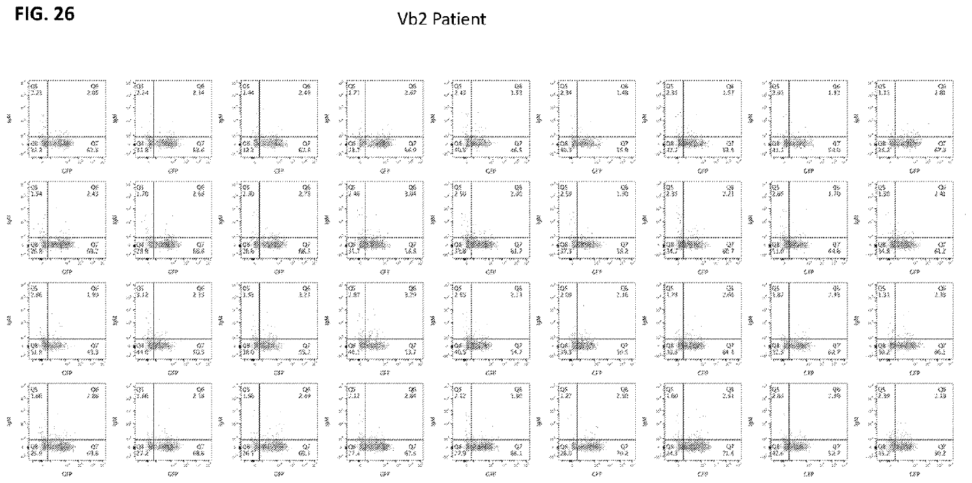

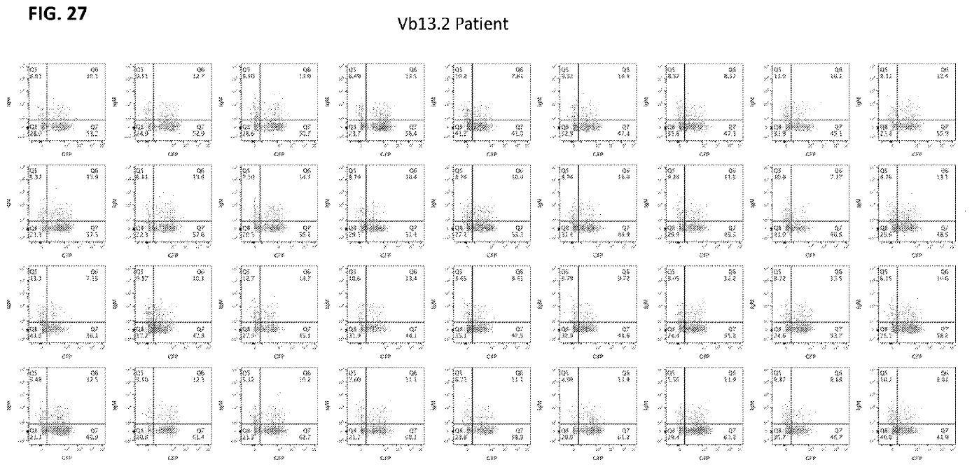

Human serum was collected from three healthy control (HC) donors (HC1, HC2 and HC3), as seen in FIGS. 22-24, respectively, a Vβ1+ CTCL patient (FIG. 25), a Vβ2+ CTCL patient (FIG. 26), and a Vβ13.2+ CTCL patient (FIG. 27), via methods disclosed in example 1. These sera samples were then incubated with a panel of all humanized pan CAR-Vβ2, CAR-CD19 and mCAR-Vβ2 T cells generated from a healthy donor.

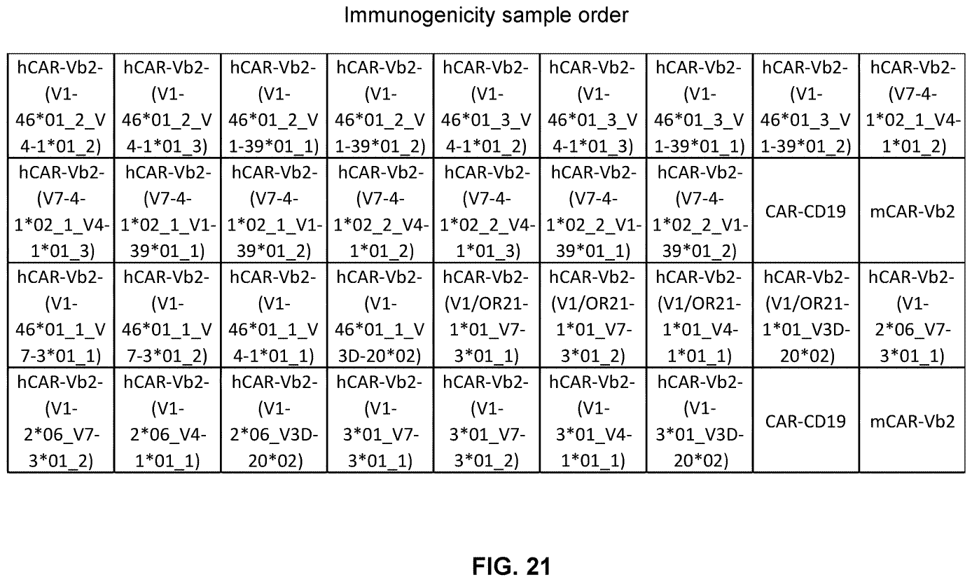

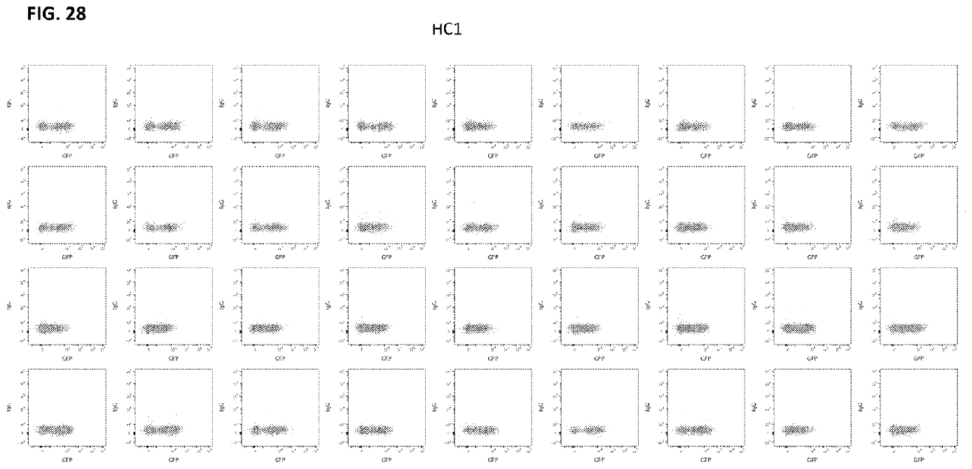

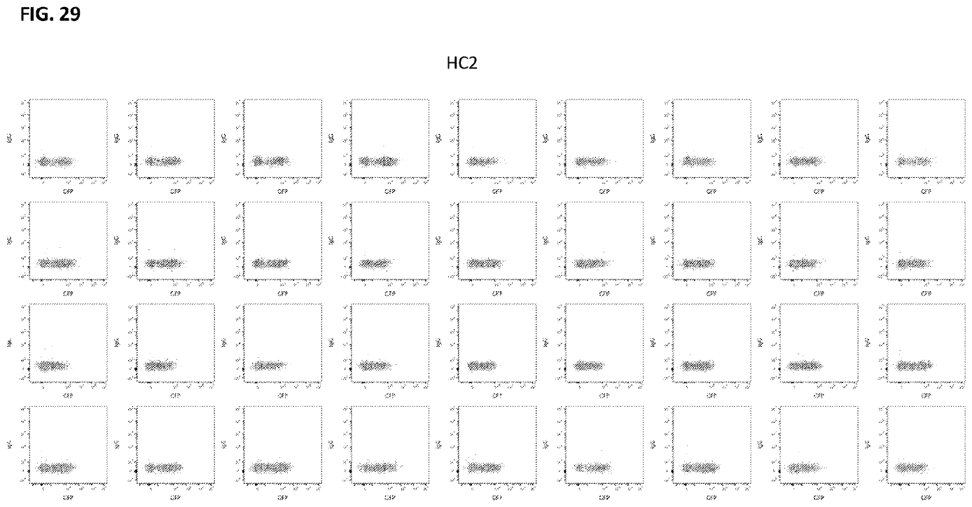

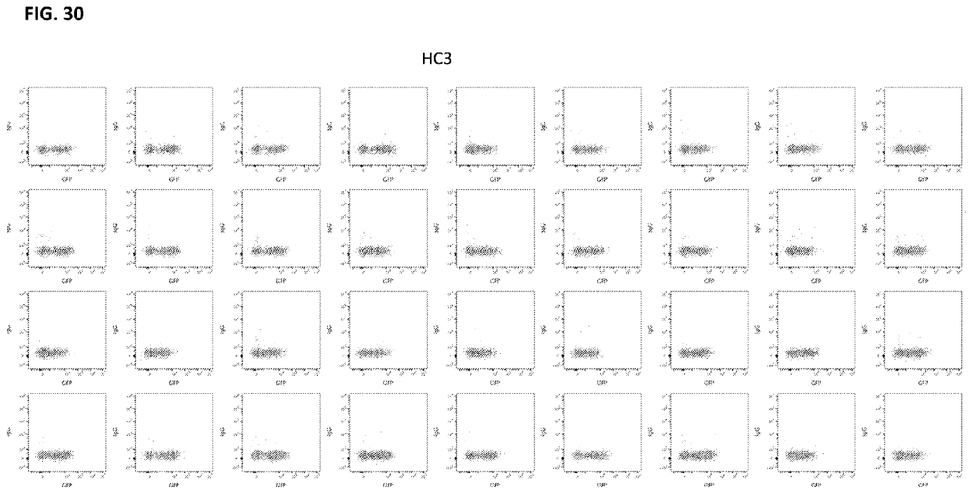

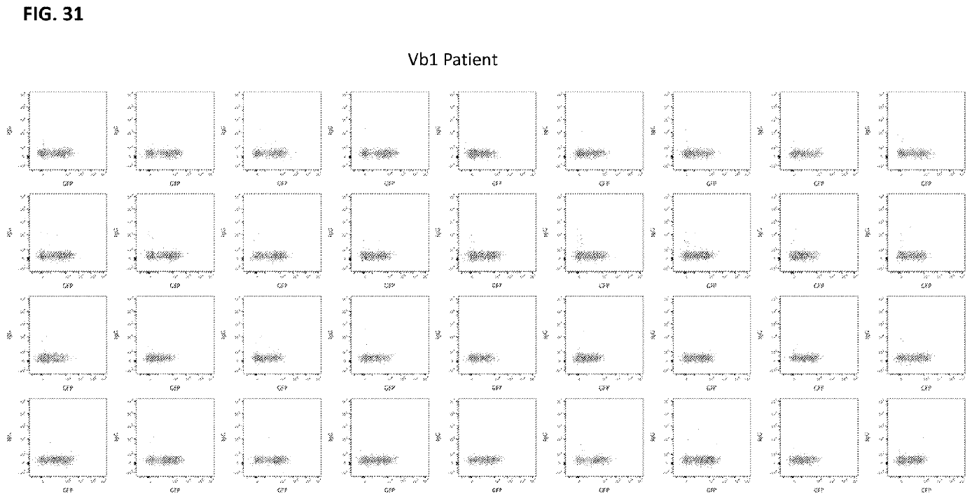

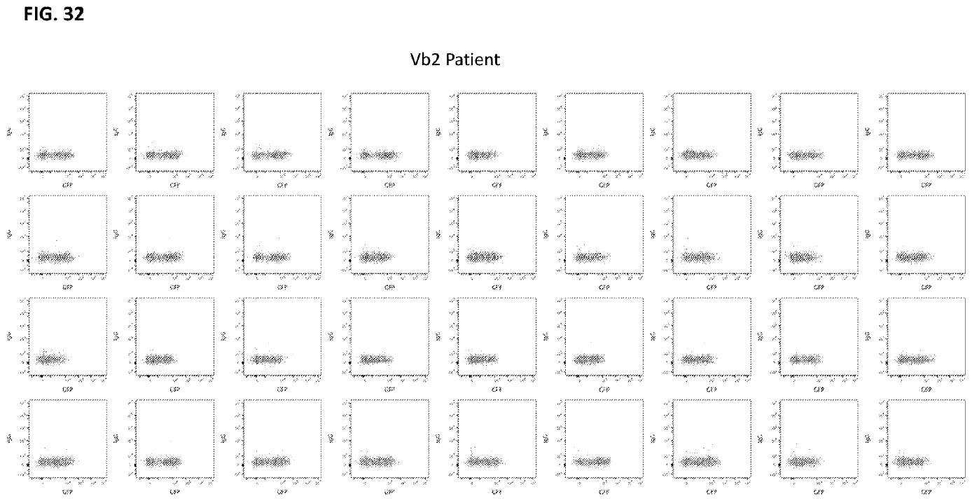

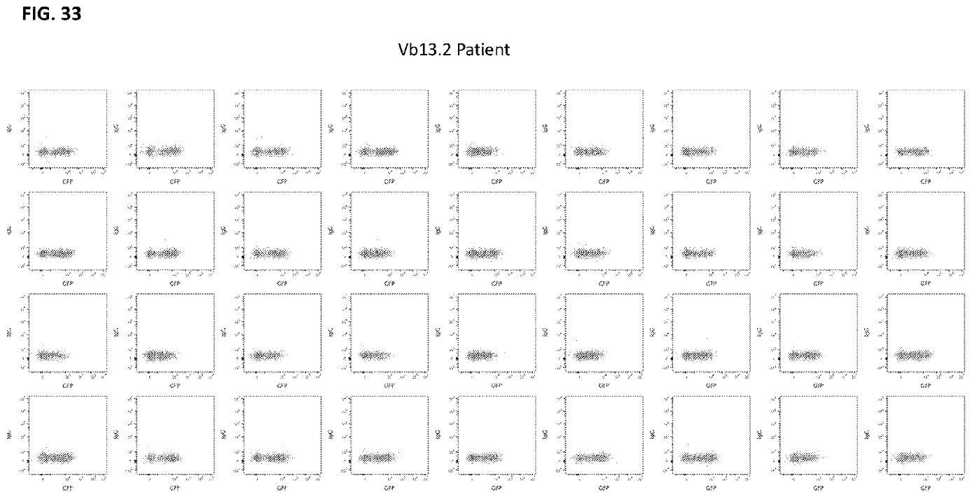

CAR cells were re-suspended in 50 μl 10× diluted serum and incubated at 4° C. for 30 minutes, followed by staining with anti-human IgG-PE and anti-human IgM-APC secondary antibody mixture at 4° C. for 20 minutes. Then, pre-existing anti-CAR or anti-allogeneic cell IgM (FIGS. 22-27) and IgG (FIGS. 28-33) of the HC 1, 2, and 3 samples (respectively FIGS. 28, 29, 30), Vβ1+ CTCL patient (FIG. 31), a Vβ2+ CTCL patient (FIG. 32), and a Vβ13.2+ CTCL patient (FIG. 33) Vβ binding potential to allogeneic CAR cells were detected by flow cytometry. FIG. 21 illustrates the alignment of each CAR-V2, CAR-CD19 and mCAR-Vβ2 T cell, to the respective flow cytometry results, as illustrated in FIGS. 22-33. A small proportion of IgM+ cells were detected in all CAR cell samples, while CAR-expressing (GFP+) and CAR-(GFP−) subpopulations showed no differential IgM+ percentage.

Furthermore, no IgG+ cells were detected, confirming the majority of allogeneic CAR cells and extracellular CAR-domains are not immunogenic, and provides evidence in support of the potential persistence of allogeneic humanized CAR-Vb2 T cells in CTCL patients.

### Long-Term In Vivo Treatment Effect of Allogeneic Humanized CAR-Vb2 T Cells

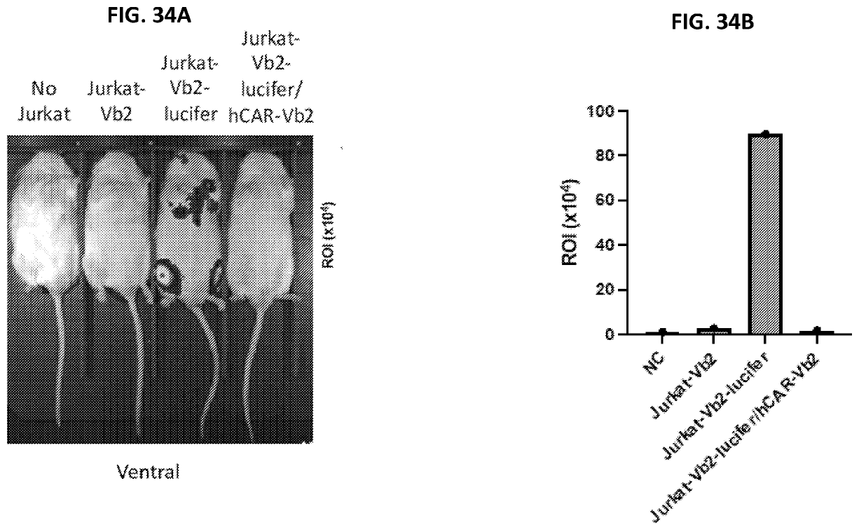

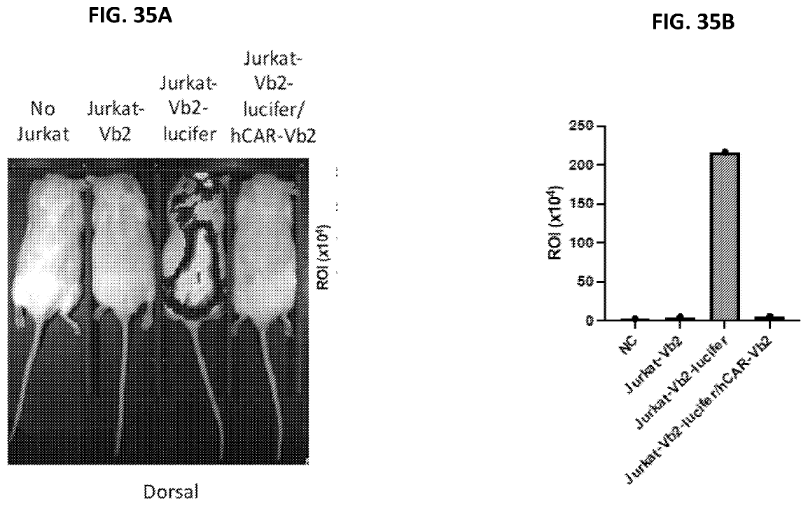

Long-term monitoring of Jurkat-Vβ2+ cells expressing luciferase or not, with or without hCAR-Vβ2 T cell treatment in NSG mice was achieved by in vivo luciferin signal detection via IVIS. 3×10⁶ Jurkat cells engineered to express TCR-Vβ2 (Jurkat-Vβ2) or TCR-Vβ2 and luciferase (Jurkat-Vβ2-lucifer) were intravenously inoculated into NSG mice. Three days later, 8×10⁶ hCAR-Vβ2 T cells were intravenously transferred into one Jurkat-Vβ2− lucifer inoculated mouse. Mice were imaged once per week by IVIS, 10 minutes after 150 ul D-luciferin/mouse intraperitoneal injection. Luciferin signal was quantified by ROI measurement. Ventral (FIGS. 34A-34B) and dorsal (FIGS. 35A-35B) positions were imaged at day 21 post Jurkat inoculation, which is also day 17 post hCAR-Vβ2 T cell treatment. Luciferin signal was shown as representative images on left panel and quantification bar charts on right panel of each of FIGS. 34A-35B. Compared to no Jurkat inoculation negative control (NC), Jurkat-Vβ2 without luciferase expression, the mouse inoculated with luciferase-expressing Jurkat-Vβ2 cells showed strong luciferin signal while the mouse inoculated with luciferase-expressing Jurkat-Vβ2 cells treated by hCAR-Vβ2 T cells showed no luciferin signal, indicating that hCAR-Vβ2 treatment is effective and persists in long-term up to three weeks in this example.

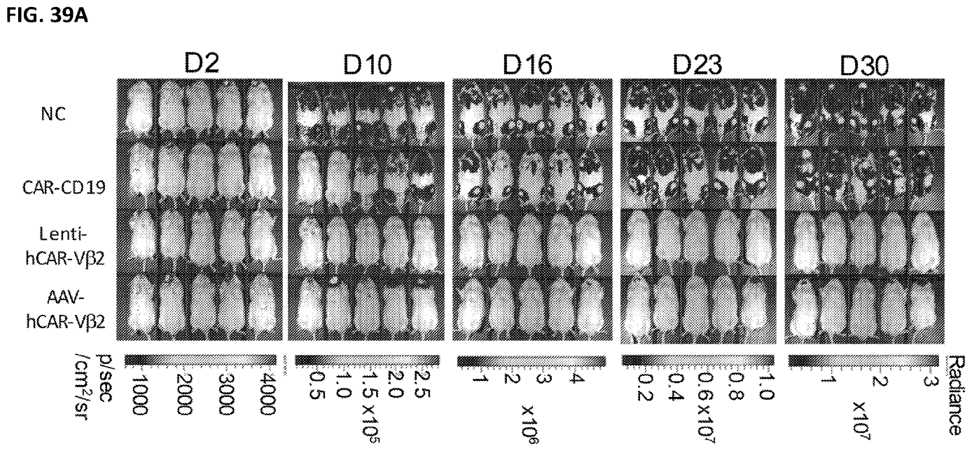

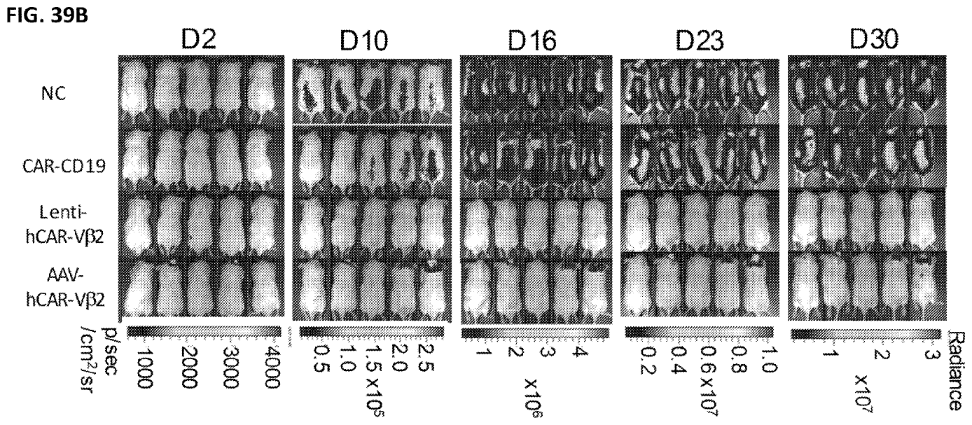

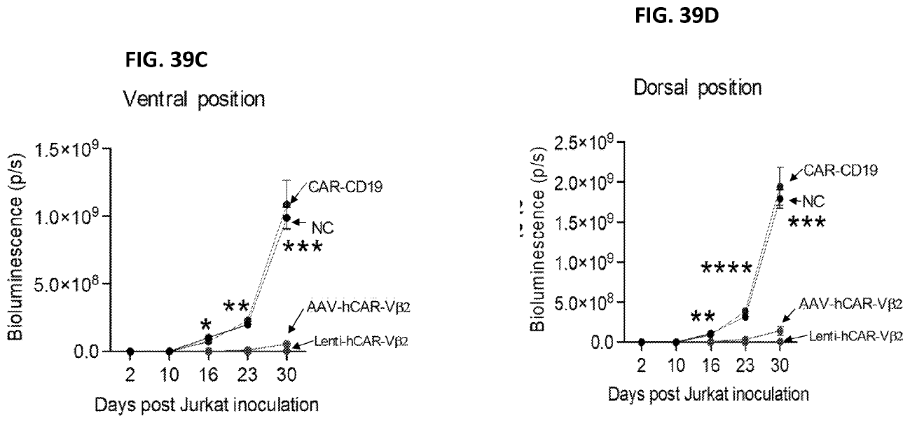

Furthermore, sustained in vivo efficacy of hCAR-Vβ2 against Vβ2+ PDX tumors is apparent as shown in FIGS. 39A-39B. Long-term monitoring (IVIS) of luciferin bioluminescence signals in Jurkat-TRBV20-1-lucifer cell-bearing NSG mice, as displayed in (FIGS. 39A and 39B) show the ventral position and dorsal position, respectively, between without treatment (NC), following a single treatment with CAR-CD19 T cells, lenti-hCAR-Vβ2 T cells, and AAV-hCAR-Vβ2 T cells. The hCAR T cells generated by both lentivirus and CRISPR-AAV systems showed strong sustained Vβ2+ Jurkat tumor cell suppression in comparison to the NC and CAR-CD19 administration.

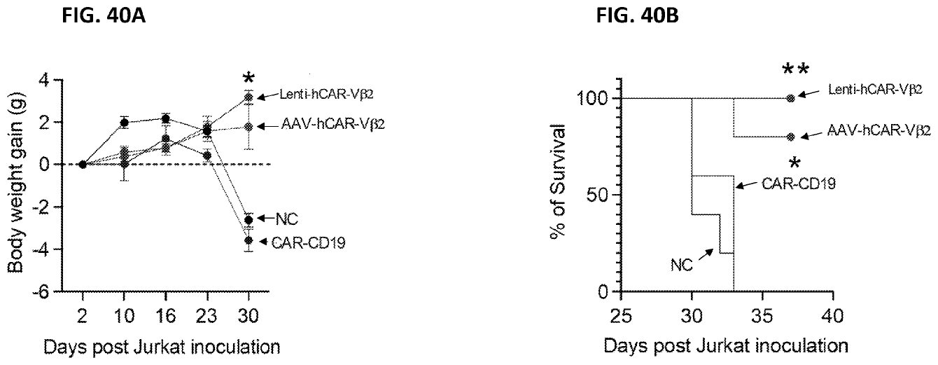

FIGS. 40A-40B both illustrate the in vivo safety and efficacy of hCAR-Vβ2 against Vβ2+ PDX tumors. The body weight and survival percentages are both from the same NSG as from FIGS. 39A-39B. These figures compare via Jurkat-TRBV20-1-lucifer cells, mice without treatment (NC) and mice following treatment with CAR-CD19 T cells, lenti-hCAR-Vβ2 T cells, and AAV-hCAR-Vβ2 T cells. The data shows that tumor bearing mice treated with hCAR-Vβ2+ T cells generated by both lentivirus and CRISPR-AAV systems displayed no body weight loss and had improved survival, consistent with the strong sustained Vβ2+ Jurkat tumor cell suppression, in comparison to NC mice and CAR-CD19 treated mice.

### Example 6. Anti-Vβ2 Antibody

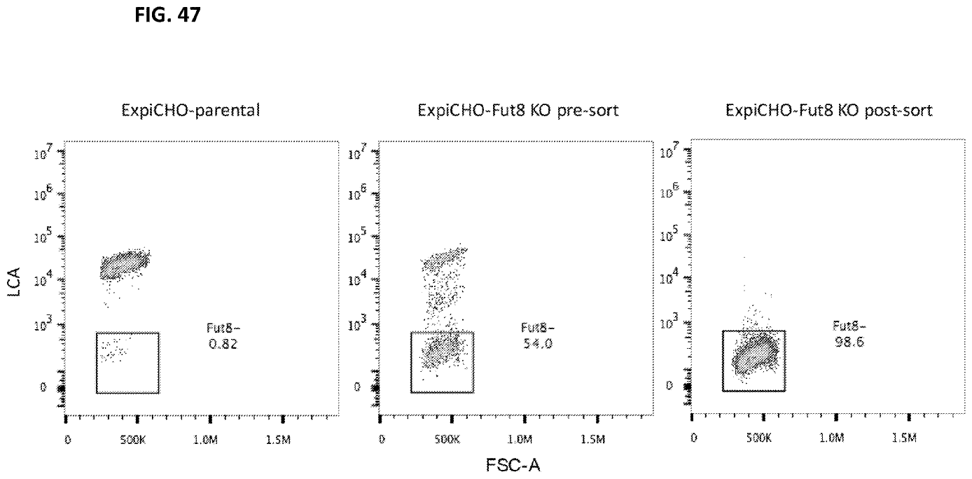

Fut8 (Fucosyltransferase 8) KO expiCHO cell line for production of anti-Vβ2 antibody with enhanced ADCC capacity is illustrated as shown in FIG. 47.

FIG. 47 further depicts the Lens Culinaris Agglutinin (LCA) expression on the surface of parental expiCHO antibody producing cells, pre-sorted expiCHO cells post Fut8 knockout (KO) or post-sorted (purified) expiCHO cells post Fut8 KO, quantified by flow cytometry. Fut8 knockout eliminates the post-translational modification of fucosylation thereby allowing expiCHO production of humanized anti-Vβ2 IgG1 antibodies with enhanced ADCC function.

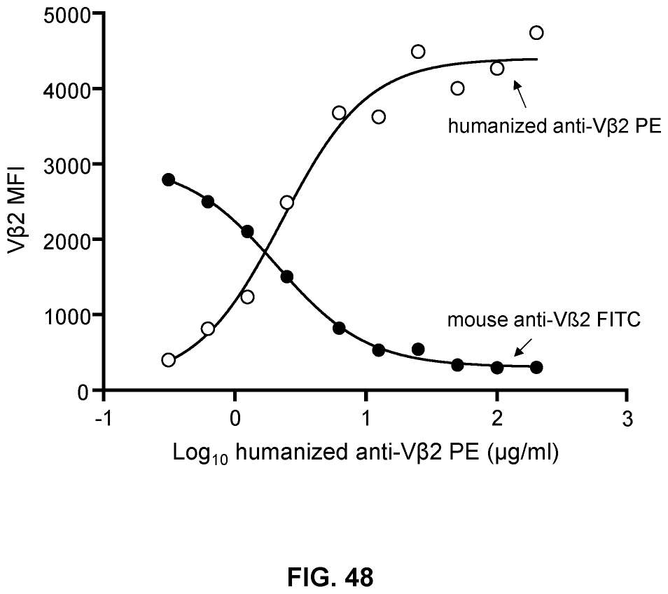

As shown in FIG. 48, an anti-Vβ2 antibody competition assay utilized 1.25 g/ml mouse anti-Vβ2-FITC antibody and was then mixed with varying concentrations of humanized anti-Vβ2-PE antibody, and used to stain CTCL cells from a Vβ2+ patient, with Vβ2 MFI quantified by flow cytometry. Humanized anti-Vβ2 IgG1 antibody showed antigen affinity that is similar to the original mouse version of the antibody.

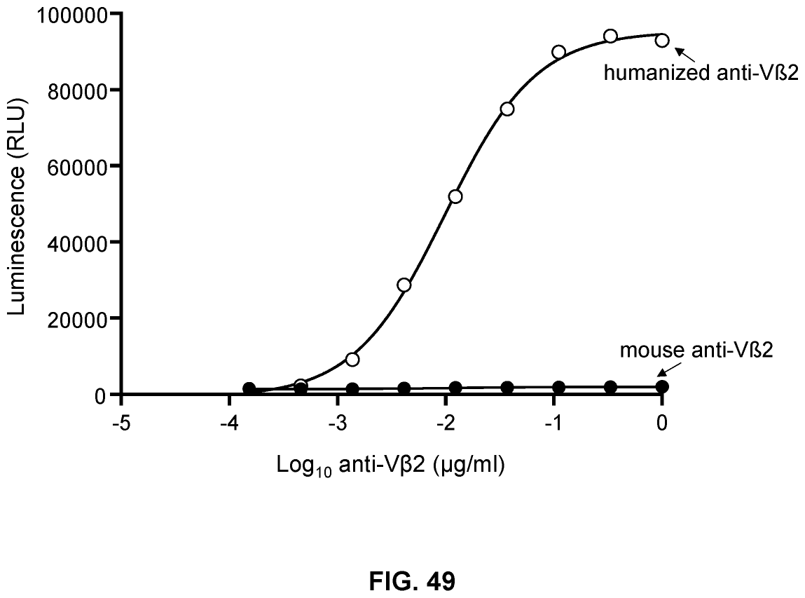

The functional ADCC assay of humanized anti-Vβ2 antibody is shown via ADCC assay luminescence signal after a 6 hour co-culture of patient derived Vβ2+ target CTCL cells and Jurkat-NFAT-luciferase effector cells mixed with 100 ng/ml of either mouse anti-Vβ2 antibody or humanized anti-Vβ2 antibody as shown in FIG. 49. Humanized anti-Vβ2 IgG1 antibody showed strong ADCC activity compared to the original mouse version of the antibody. Furthermore, the humanized anti-Vβ2 antibody enables NK mediated ADCC of Jurkat-TRBV20-1 cells in vitro.

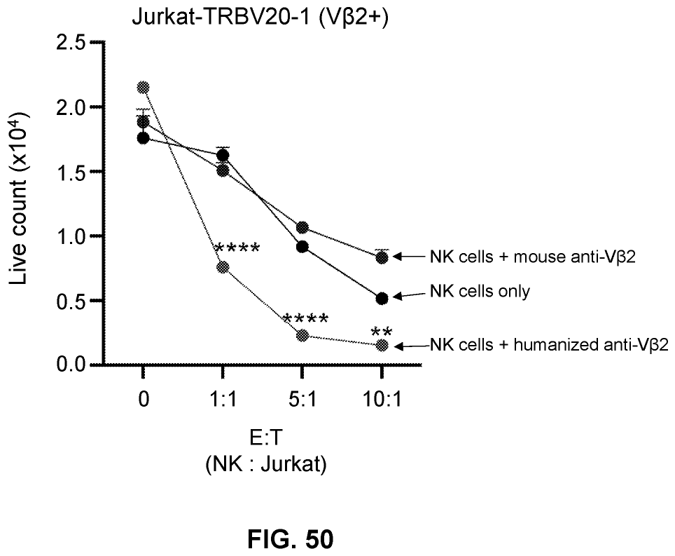

As illustrated in FIG. 50, live Jurkat-TRBV20-1 target cell counts, after overnight co-culture with effector NK cells from a healthy donor at different E:T ratios without antibody addition, mixed with 100 ng/ml mouse anti-Vβ2 antibody, and humanized anti-Vβ2 antibody, were then quantified by flow cytometry. Humanized anti-Vβ2 IgG1 with the enhanced ADCC modification showed superior target killing efficiency compared to NK cells alone.

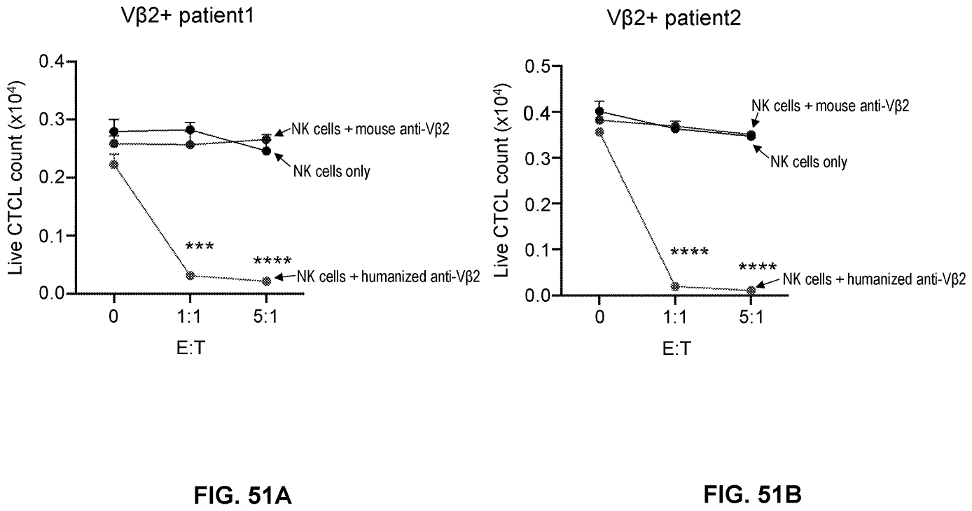

The humanized anti-Vβ2 antibody enabled NK mediated ADCC of patient derived Vβ2+ CTCL cells. As seen in FIGS. 51A-51B, the PBMC from Vβ2+ CTCL patient 1 (FIG. 51A) or from Vβ2+ CTCL patient 2 (FIG. 51B) were cultured overnight with NK effector cells from a healthy donor at different E:T ratios without antibody addition, or with the addition of mouse anti-Vβ2 antibody, or humanized anti-Vβ2 antibody. Then, live CTCL cell counts were determined via flow cytometry. Humanized anti-Vβ2 IgG1 with the enhanced ADCC modification showed efficient killing of Vβ2+ CTCL cells from patients.

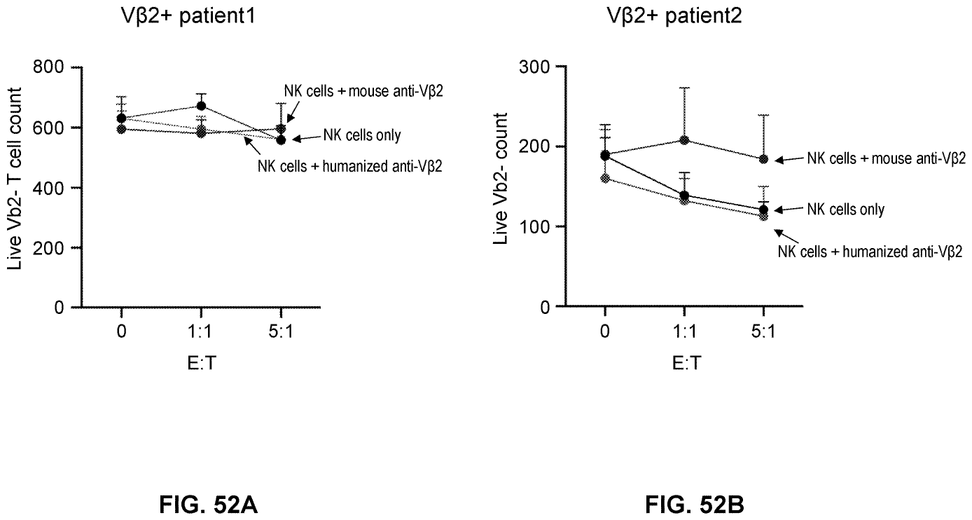

As shown in FIGS. 52A-52B, the humanized anti-Vβ2 directed NK mediated ADCC is specific for Vβ2+ CTCL cells. The PBMC from either Vβ2+ CTCL patient 1 or patient 2 were cultured overnight with NK effector cells from a healthy donor at different E:T ratios without an antibody addition or with the addition of mouse anti-Vβ2 antibody, or humanized anti-Vβ2 antibody, and then live Vβ2-negative normal T cell counts were quantified by flow cytometry. Humanized anti-Vβ2 IgG1 with the enhanced ADCC modification enabled specific killing of Vβ2+ CTCL cells from patients, without effecting Vβ2-negative T cells from the same patients.

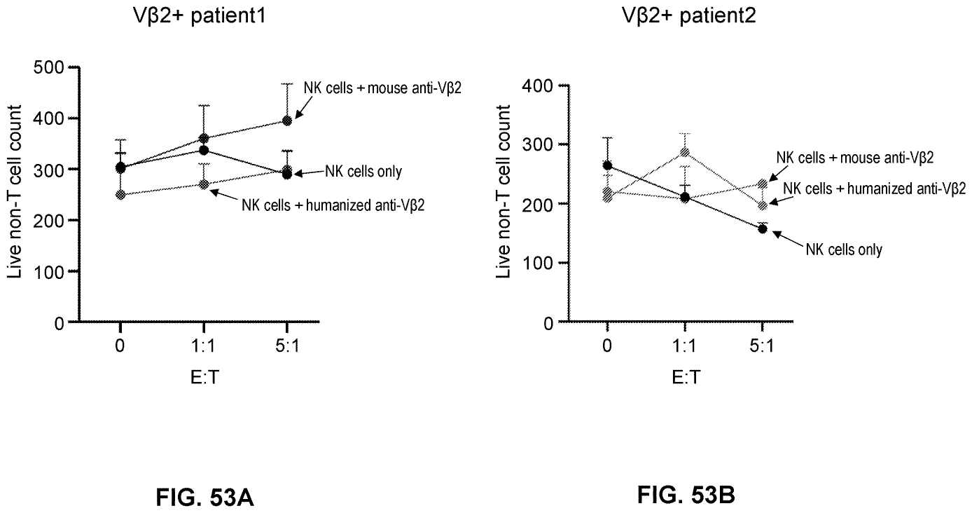

Further evidence of the specificity of humanized anti-Vβ2 directed NK mediated ADCC is shown in FIGS. 53A-53B. PBMC from Vβ2+ CTCL patient 1 (FIG. 53A) or from Vβ2+ CTCL patient 2 (FIG. 53B) was cultured overnight with NK effector cells from a healthy donor at different E:T ratios, without the antibody addition, or with addition of mouse anti-Vβ2 antibody, or humanized anti-Vβ2 antibody. Then, the live non-T cell PBMC counts were quantified by flow cytometry. Humanized anti-Vβ2 IgG1 with the enhanced ADCC modification showed specific killing of Vβ2+ CTCL cells from patients, without effecting non-T cells from the same patients.

### Example 7. In Vivo Validation of Humanized Anti-Vβ2 Antibody

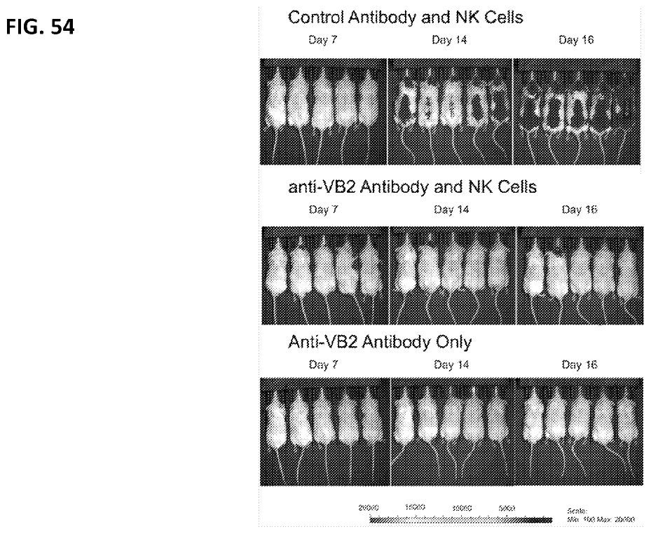

T cell lymphoma was successfully treated with an anti-Vβ2 humanized therapeutic antibody (BV7-4-1*02_1_V1-39*01_2), as shown in FIG. 54.

The established model of Vβ2+ T cell lymphoma as depicted within FIG. 54 was generated via injection of a clonal population of Jurkat target cells that were engineered to express the T cell receptor Vβ2. Groups of mice were subsequently treated once a week, on days 3 and 9, with control antibody plus human natural killer (NK) cells (see top row of FIG. 54), anti-Vβ2 antibody plus NK cells (see middle row of FIG. 54), or anti-Vβ2 antibody solely administered (see bottom row of FIG. 54). Both groups of mice that received the anti-Vβ2 antibody (see middle and bottom row) showed significantly less luminescence compared to mice that received the control antibody plus NK cells (see top row). This indicated complete to near complete elimination of the Vβ2+ Jurkat target cells in mice that received the anti-Vβ2 antibody compared to the mice that received the control antibody.

### Example 8. Validation of Mouse Anti-Vβ CAR and Antibody Sequences by CD69 Activation

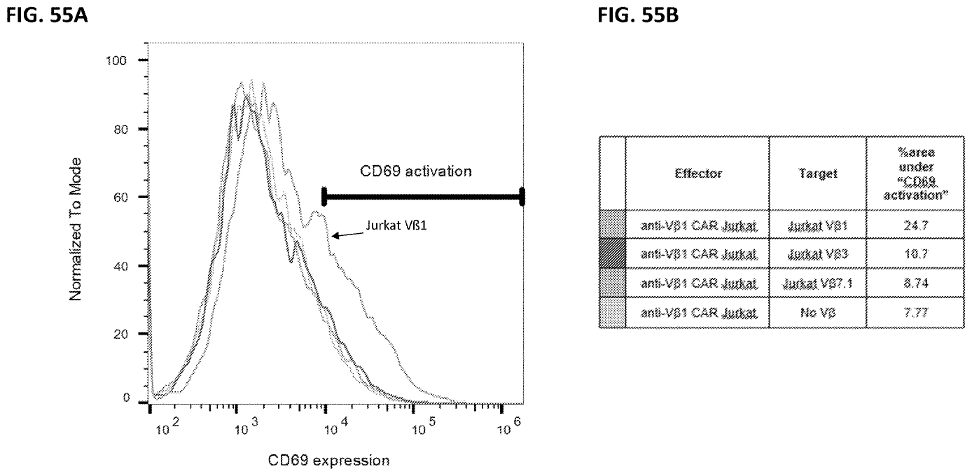

Validation of mouse anti-Vβ1 CAR (SEQ ID NO: 629) and antibody sequence activity was then measured by CD69 activation, as shown in FIGS. 55A and 55B. The jurkat effector cells expressing an anti-Vβ1 CAR were mixed with Jurkat target cells that were not expressing any Vβ, or stably expressing Vβ1 (see far right data line most proximal to “CD69 activation” of FIG. 55A), Vβ3, or Vβ7.1. As shown in FIGS. 55A and 55B, CD69 expression was upregulated in Jurkat effector cells only when Vβ1 was present on target cells. As shown in FIG. 55B, the highest % area under the “CD69 activation” was the anti-Vβ1 CAR Jurkat targeting Jurkat Vβ1, with a value of 24.7%. This confirms that this anti-Vβ1 CAR is specific to Vβ1, and that the antibody sequence for the anti-Vβ1 single-chain variable fragment on the CAR, which was derived from a mouse anti-Vβ1 antibody sequence, was validated.

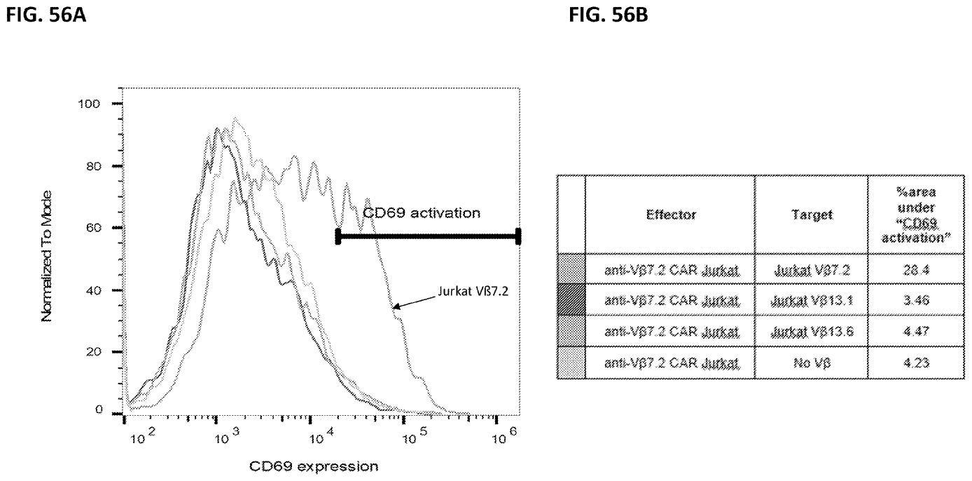

Validation of mouse anti-V87.2 CAR (SEQ ID NO: 630) and antibody sequence activity was then measured by CD69 activation, as shown in FIGS. 56A and 56B. The jurkat effector cells expressing an anti-Vβ1 CAR were mixed with Jurkat target cells that were not expressing any Vβ, or stably expressing Vβ7.2 (see far right data line most proximal to “CD69 activation” of FIG. 56A), Vβ13.1, or Vβ13.6. As shown in FIGS. 56A and 56B, CD69 expression was upregulated in Jurkat effector cells only when Vβ1 was present on target cells. As shown in FIG. 56B, the highest % area under the “CD69 activation” was the anti-Vβ7.2 CAR Jurkat targeting Jurkat Vβ7.2, with a value of 28.4%. This confirms that this anti-Vβ7.2 CAR is specific to Vβ7.2, and that the antibody sequence for the anti-Vβ7.2 single-chain variable fragment on the CAR, which was derived from a mouse anti-Vβ1 antibody sequence, was validated.

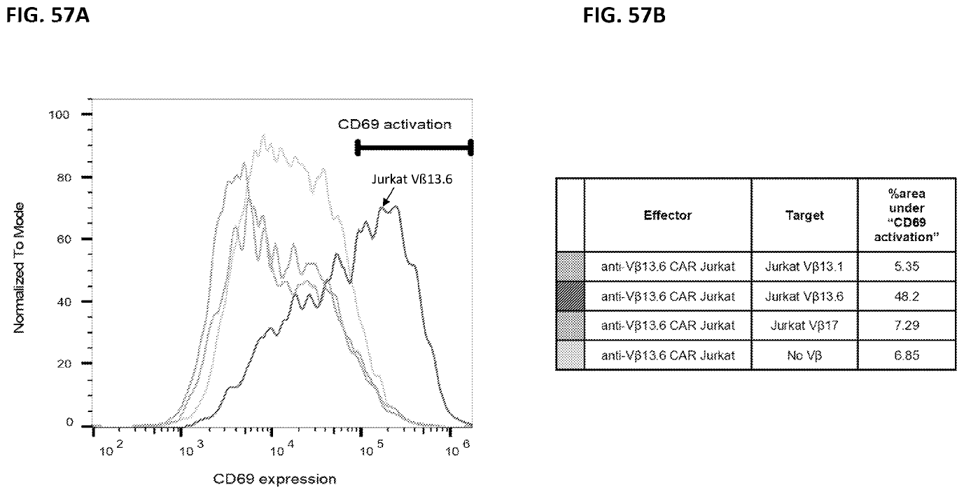

Validation of mouse anti-Vβ13.6 CAR (SEQ ID NO: 631) and antibody sequence activity was then measured by CD69 activation, as shown in FIGS. 57A and 57B. The jurkat effector cells expressing an anti-Vβ13.6 CAR were mixed with Jurkat target cells that were not expressing any Vβ, or stably expressing Vβ13.6 (see far right data line most proximal to “CD69 activation” of FIG. 57A), Vβ13.1, or Vβ17. As shown in FIGS. 57A and 57B, CD69 expression was upregulated in Jurkat effector cells only when Vβ13.6 was present on target cells. As shown in FIG. 57B, the highest % area under the “CD69 activation” was the anti-Vβ13.6 CAR Jurkat targeting Jurkat Vβ13.6, with a value of 48.2%. This confirms that this anti-Vβ13.6 CAR is specific to Vβ13.6, and that the antibody sequence for the anti-Vβ13.6 single-chain variable fragment on the CAR, which was derived from a mouse anti-Vβ313.6 antibody sequence, was validated.

### Materials and Methods Used in the Examples

### Methods, Samples, and Plasmids

Peripheral blood (PBMC) from CTCL patients and healthy donors were collected in lithium heparin tubes at the Yale Cancer Center in according with regulations. The fresh peripheral blood was 1:1 diluted in room temperature (RT) 1×PBS and layered on Ficol-Plus in SepMate (Stem Cell) centrifuge tubes. The buffy coat was harvested according to SepMate instructions. Following extensive PBS washing, the PBMC was then counted and then either directly cryopreserved in liquid nitrogen or immediately used for pan T-cell, CD4+ T-cell, CD8+ T-cell, or CTCL cell isolation by untouched MACS negative selection kits (Miltenyi).

For CTCL cell isolation, anti-CD26-biotin and/or anti-CD7-biotin were added to remove CD26+ and/or CD7+ cells based on the clinical phenotypes of the malignant CTCL cells in each patient. Isolated T-cell subtypes were either directly cryopreserved in liquid nitrogen or cultured in T-cell medium (RPMI) supplied with 10% heat-inactivated FBS, 20 ng/ml rhIL2, 5 ng/ml rhIL7 and 10 ng/ml rhIL15 (cytokines from R&D Systems).

The mCAR-Vβ2 expressing lentiviral vector plasmid, was generated via BbsI digestion of pSLCAR-CD19-BBz (Addgene #135992), which was subsequently followed by NEBuilder HiFi DNA Assembly with gBlock (IDT) which contained scFv targeting TRVB2. Further stabilization of the scFv structure was required. Therefore, amino acids at Kabat positions VH44 and VL100 were substituted to Cysteine.

The TCR-Vβ2 lentiviral plasmid was generated from pAW13.lentiguide.mcherry (addgene #104375). Afterwards, the U6-gRNA scaffold fragment was cut out by KlfJ/BsmBI double digestion, and the ends of linearized plasmid was repaired using a Quick Blunting kit (NEB). Thereafter, the plasmid was then self-ligated by T4 ligase (NEB). The plasmid was then double digested by BsiWI/MscI, followed by NEBuilder HiFi DNA Assembly with TCR-Vβ2 PCR product from T-cells of a Vβ2+ patient and gBlock (IDT) containing TCR-Vβ2 3′ overlapping+a P2A peptide+mcherry 5′ overlapping.

Humanized CAR-Vβ2 (hCAR-V2) plasmids were generated via BbsI digestion of pSLCAR-CD19-BBz, followed by NEBuilder HiFi DNA Assembly with humanized VH and VL gBlocks (IDT) containing VH44 and VL100 cysteine replacements.

The pSLCAR-CD19-BBz (#135992), pAW13.lentiguide.mcherry (#104375), pMD2.G (#12259) and psPAX2 (#12260) plasmids were purchased from Addgene.

The mCAR-Vβ2 expressing AAV vector used a pAAV.CMV.PI.EGFP.WPRE.bGH plasmid (Addgene #105530). The left and right homology arms of the human TRAC region and CAR-GFP fragment, as shown in FIG. 18, were generated via PCR using gDNA from T cells of a healthy donor and CAR-containing lentiviral plasmid as templates, respectively. The WPRE structure from the AAV backbone was then removed, followed by the sequential insertion of (1) left homology arm, (2) T2A, (3) CAR, (4) a P2A peptide, (5) GFP, and (6) right homology arm to generate the CAR-expressing vector as pAAV.pTRA.T2A.CAR.P2A.GFP.bGH.

To produce the AAV, AAV transfer plasmid and pDGM6 were co-transfected into 293T cells that were harvested three days later. AAV in transfected 293T cells were then extracted and purified by AAVpro purification kit (TaKaRa, Cat. 6675). The respective virus titer was then measured by qPCR.

To generate CAR-expressing T cells by AAV vector, T cells isolated from a healthy donor were activated by anti-CD3/CD28 beads at 1:1 ratio for two days, and beads were removed followed by RNP-dependent TRAC/B2M/CIITA triple KO. Following a 1 hour rest in complete T cell culture medium, T cells were resuspended in serum-free T cell culture medium containing cytokines and AAV (MOI=2×10⁴) at 1×10⁷ cells/ml concentration and incubated at 37° C. for 30 minutes. Complete T cell culture mediums containing 10% FBS, cytokines and homology recombination enhancer cocktail (M3814/TSA/XL413) were then added to a cell concentration of 1×10⁶/ml. Homology recombination enhancer cocktail was then removed following an overnight incubation period. The one day culture was then replaced with fresh complete T cell medium. Remaining CD3+ cells were then removed as described above for the two to three day post KO methodology.

### Generation of Jurkat Cell Pools Stably Expressing Either a Chimeric Antigen Receptor (CAR) or Vβ.

The Luc gene encoding luciferase was first integrated into the genome of Jerkat E6-1 cells (ATCC) via lentiviral transduction. Next, the endogenous T cell receptor (TCR) was knocked out, followed by genomic integration (via lentiviral transduction) of a CAR construct (for effector cells) or Vβ gene (for target cells). The CAR was linked to GFP, whereas the Vβ was linked to mCherry, both via a “2A” peptide linker.

### T Cell Lymphoma Mouse Model, Antibody Treatment, and Imaging.

A single Jurkat target cell with high expression of mCherry, luciferase, and Vβ2 was selected from a pool to establish a clonal population. The clonal population was then injected into NSG mice (JAX) on day 0. On days 3 and 9, the mice were either treated with control antibody (anti-HA) plus human natural killer (NK) cells, anti-Vβ2 antibody plus NK cells, or anti-Vβ2 antibody alone. On days 7, 14, and 16, the mice were injected with luciferin and imaged on the IVIS® Spectrum In Vivo Imaging System (PerkinElmer).

### Animals

NOD.Cg-Prkdc^{sicd} I12rg^{tmlWjl}/SzJ (NSG) mice were purchased from the Jackson Laboratory. All of the in vivo studies were approved by the Yale Institutional Animal Care and Use Committee. Mice were bred and maintained under specific pathogen-free conditions with food and water provided ad libitum.

### Lentivirus Production

293T-cells were seeded overnight and then transfected by lentiviral vector, specifically pMD2.G and psPAX2 in lipofectamine2000. Following 6-8 hours of plasmid addition, fresh medium was then added. 24 hours later, the first supernatant was collected and stored at 4° C. Fresh medium was added. 24 hours later, the second supernatant was collected and combined with first one, followed by 0.4 um filtering. Aliquots are held at −80° C. for long-term storage.

### Lentivirus Transduction

Jurkat cells were re-suspended in lentiviral supernatant with 8 ug/ml polybrene. Spinfection was performed 90 minutes×1200 g at 32° C., followed by changing fresh medium. Primary human T-cells freshly isolated or overnight recovered in cytokine-medium after thawing were stimulated by anti-CD3/CD28 beads at 1:1 ratio for 2 days. Then beads were magnetically removed and activated T-cells were re-suspended in lentiviral supernatant containing cytokines and 8 ug/ml polybrene. Spinfection was performed 120 minutes×2000 g at 32° C., followed by changing fresh medium containing cytokines. After overnight culture, T-cells were centrifuged and re-suspended in fresh medium containing cytokines for further expansion.

### Gene Knockout

Knock out (KO) was accomplished via sgRNAs targeting human TRAC, B2M and CIITA, which were purchased from IDT. Culture medium was pre-warmed in a 37° C. incubator. Ribonucleoprotein (RNP) complex composed of Cas9 protein and sgRNA was formed at room temperature for 20 minutes. The supernatant was then aspirated completely from the cell pellet. The cell pellet subsequently was then re-suspended in 20 ul P3 buffer (Lonza). Cell suspension, RNP complex, and enhancer (IDT) were mixed thoroughly and then transferred into 16-well strip tubes (Lonza).

Electroporation was performed in Lonza 4D-x unit with program EO-115. Approximately 100 ul of pre-warmed culture medium was immediately added to the cells after electroporation, and then transferred completely into pre-warmed medium for culture.

Patient derived CTCL cells expressing Vβ2 (N=3) or Vβ13.2 (N=1) were cultured overnight with mCAR-Vβ2 T-cells at effector-to-target (E:T, mCAR-Vβ2 effector: CTCL target) ratios of 0, 1:1 and 5:1. Live CTCL cells were then quantified using flow cytometry. mCAR-Vβ2 with TRAC KO show enhanced specificity and highly effective killing of Vβ2+ CTCL cells.

### CD3+ T-Cell Depletion

Two days post TRAC KO, anti-CD3/CD28 beads were removed from the T-cells. The remaining CD3+ population was removed using MACS anti-CD3 microbeads and LD columns (Miltenyi), following the manufacturer's protocol. TRAC KO CD3− T-cells were collected from column flow through.

### CAR-Cell Generation

Isolated CD8 T-cells or pan-T-cells were activated by anti-CD3/CD28 beads for one day. Then the beads were removed, followed by gene (TRAC, B2M or CIITA) KO. After a 2-hour rest in culture medium, new anti-CD3/CD28 beads were added to stimulate the cells for one additional day. CAR-containing lentivirus transduction was then performed as described above. Transduced CAR-cells were further expanded and differentiated in cytokine containing medium for 7-10 days before use in killing studies. Fresh cytokine-containing medium was added every two to three days.

### Flow Cytometry

Cells were re-suspended in 100 ul 1×PBS containing FcR block (BD) and Aqua Live/Dead fixable dye for 10 minutes at room temperature (RT). Without wash, 100 ul antibody mixture in 1×PBS was added for 30 minutes at 4° C. Following a wash in 1×PBS containing 2% FBS, cells were re-suspended in 200 ul 1×PBS containing 2% FBS and 10 ul counting beads for flow cytometry detection (Thermofisher) were added.

### Antibody Mass Spectrometry (MS)

Antibody targeting human TCR Vβ2 was purchased from Beckman Coulter. High resolution MS was performed by Rapid Novor. Amino acid sequences of the antibody generated by MS were annotated to identify framework and CDR regions of variable domains of heavy and light chains.

### In Vivo Imaging

In vivo imaging utilized 3×10⁶ Jurkat cells, which were engineered to express TRBV20-1 (Vβ2) and luciferase (Jurkat-TRBV20-1-lucifer). These cells were intravenously inoculated into NSG mice. Three days later, 1×10⁷ lenti-CAR-CD19, lenti-hCAR-Vβ2 or AAV-hCAR-Vβ2 T cells were intravenously injected into the mice. Tumor burden was then assessed once per week by IVIS (PerkinElmer) bioluminescence imaging, following a 10 minute period after receiving 150 μl D-luciferin/mouse (i.p.). Luciferin signals from both ventral and dorsal positions were quantified by ROI measurement (Living Image). Mouse body weight was monitored once per week initially and then twice per week when tumors were evident. A≥20% body weight loss was regarded as the endpoint for survival curve calculation.

### In Vitro Killing Assay

Target T-cells were stained with cell trace violet dye (Thermofisher) following the manufacturers instruction. 1×10⁴ stained targeT-cells were then seeded in 96-well U bottom plates. Effector CAR-cells were mixed with the seeded target T-cells at effector to target (E:T) ratios of 0, 1:1 and 5:1. After overnight culture, cells were re-suspended in 1×PBS containing 2% FBS and 10 ul CountBright Absolute Counting Beads (Thermofisher) for flow cytometry.

### In Vivo Killing Assay

On day 0 (D0), NSG mice were inoculated intravenously with 3×10⁶ Jurkat-Vβ2 cells or 5×10⁶ total CD4 T-cells from a Vβ2+ patient. On day 3 (D3), 8×10⁶ allogeneic mCAR-Vβ2 T-cells per mouse were adoptively transferred i.v. into NSG mice in treatment groups. Control mice did not receive mCAR-Vβ2 T-cells. On day 10 (D10), mice were euthanized and dissected to quantify remaining T lymphoma cells and mCAR-Vβ2 T-cells in spleen, bone marrow (BM) and blood by flow cytometry.

### Mouse Dissection and Cell Isolation

Mice were euthanized by CO₂ inhalation. Blood was collected by cardiopuncture into EDTA-coated collection tubes. RBC lysis buffer (Biolegend) was added for 10 minutes at RT. Spleens were smashed through a 70 um strainer, followed by RBC lysis buffer incubation for 5 minutes at RT. Femur and tibia were isolated, the ends of each bone were removed and the bone marrow was flushed out using a syringe containing 1×PBS plus 2% FBS, and then smashed through a 70 um strainer, followed by RBC lysis buffer incubation for 5 minutes at RT. Isolated cells were re-suspended in 1×PBS containing 2% FBS and kept on ice for further use.

### In Silico Humanization Design

The BioPhi in silico modeling algorithm, or a third-party contractor (mAbvice) were used for humanization design. In BioPhi, humanization settings were selected with Chothia Numbering and CDR definition. Humanization method chose either ‘Sapiens’ or ‘CDR Grafting’. OASis prevalence threshold chose either relaxed (over 10%) or strict (over 90%).

Cytokine Optimization for hCAR-V 2 T Cell Expansion In Vitro

One condition without any cytokines and 20 conditions with different cytokine combinations were then subsequently tested (see FIG. 41). The final concentration of each cytokine was measured as follows: IL2 (5 ng/ml, R&D), IL7 (5 ng/ml, R&D), IL12 (10 ng/ml, R&D), IL15 (10 ng/ml, R&D), IL18 (6.25 ng/ml, R&D) and IL21 (20 ng/ml, R&D). Adeno Associated Virus (AAV) transduced triple KO hCAR-Vβ2 T cells from a healthy donor were equally divided among the 21 conditions in triplicate. Live cells and viability were counted by trypan blue staining (obtained from Countess Cell Counter, Thermofisher) and cells were sub-cultured in fresh cytokine medium at 1×10⁵ cells/200 μl every 2-3 days. Following 7 days of cytokine expansion, an aliquot of hCAR-Vβ2 T cells was then used for flow cytometry detection (as described above) of surface phenotype and intracellular cytokine production following a 6-hour Jurkat-TRBV20-1 cell stimulation (E:T=1:1).

In addition, separate aliquots of 1×10⁴ hCAR-Vβ2 T cells were repeatedly stimulated by 2×10⁴, 5×10⁴ and 1×10⁵ Jurkat-TRBV20-1 cells at day 0, 1 and 2 of co-culture, respectively. Co-cultured cells were collected for flow cytometry at day 1, 2, 3 and 4 of co-culture to evaluate the number of live Jurkat-TRBV20-1 cells and hCAR-Vβ2 cells, and surface phenotype changes of hCAR-Vβ2 cells at the end of 4 days co-culture. After day 12 of cytokine expansion, hCAR-Vβ2 T cells were then used for flow cytometry detection of surface phenotype, and intracellular cytokine production at resting status without CAR engagement.

### Antibody Competition Assay

Vβ2+ CTCL cells from one patient were stained with a mixture of 1.25 g/ml mouse anti-Vβ2-FITC and varying concentrations of purified humanized anti-Vβ2 IgG1, followed by anti-human IgG Fc-PE staining. The relative affinity of humanized anti-Vβ2 IgG1 to the original mouse anti-Vβ2 antibody was estimated by the concentration of humanized anti-Vβ2 IgG1 used to reach 50% of maximal mouse anti-Vβ2-FITC intensity.

### ADCC Reporter Bioassay

The Promega ADCC reporter bioassay kit was used to assess ADCC activity. In brief, 1×10⁴ Vβ2+ CTCL cells from one patient were mixed with 1×10⁴ effector Jurkat-NFAT-luciferase effector cells provided by the kit and varying concentrations of mouse anti-Vβ2 antibody or humanized anti-Vβ2 IgG1. After 6 hours co-culture at 37° C. the assay reaction was quantified (Victor X-Light, PerkinElmer).

### In Vitro ADCC of NK Cells Mediated by Humanized Anti-V 2 IgG1

Peripheral blood mononuclear cells (PBMC) isolated from two Vβ2+ CTCL patients were stained with CellTrace Violet dye (Thermofisher) following the manufacturer's instruction. 1×10⁴ stained PBMC or Jurkat-TRBV20-1 cells were then seeded in 96-well U bottom plates. Purified effector NK cells from a healthy donor were mixed with the seeded target cells at effector to target (E:T) ratios of 0, 1:1, 5:1 or 10:1. Then 100 ng/ml mouse anti-Vβ2 antibody or humanized anti-Vβ2 IgG1 was added into cell mixture. After overnight culture, cells were re-suspended in 1×PBS containing 2% FBS and 10 μl CountBright Absolute Counting Beads (Thermofisher) for flow cytometry.

### CD69 Assay

Following confirmation of stable integration of the CAR construct or Vβ gene via flow cytometry, le5 cells from each cell pool were mixed in a well of a 96-well round-bottom plate, and incubated overnight at 37° C. and 5% CO₂ in a humidified incubator. Approximately 16 hours later, the cells were collected, stained for viability with a Zombie UV dye (BioLegend, catalog #423107) and for CD69 with an anti-CD69 antibody conjugated to Brilliant Violet 711 dye (BioLegend, catalog #310944), fixed in 1% paraformaldehyde, and then analyzed on a CytoFLEX LX flow cytometer (Beckman Coulter). The gating strategy was as follows: lymphocyte gate→singlets→live cells→GFP-positive and mCherry-negative cells. The CD69 gate was drawn based on the baseline signal in the Jurkat target cells that had their endogenous TCR knocked out.

TABLE 2
Sequences

| SEQ ID | | |
|---|---|---|
| NO | Description | Sequence |
| SEQ ID | AAV Nucleotide | TCTATCACGGCCATATTCTGGCAGGGTCAGTGGCTCCAACTAACATTTG |
| NO: 1 | Sequence | TTTGGTACTTTACAGTTTATTAAATAGATGTTTATATGGAGAAGCTCTCA |
| | | TTTCTTTCTCAGAAGAGCCTGGCTAGGAAGGTGGATGAGGCACCATATT |
| | | CATTTTGCAGGTGAAATTCCTGAGATGTAAGGAGCTGCTGTGACTTGCT |
| | | CAAGGCCTTATATCGAGTAAACGGTAGTGCTGGGGCTTAGACGCAGGT |
| | | GTTCTGATTTATAGTTCAAAACCTCTATCAATGAGAGAGCAATCTCCTG |
| | | GTAATGTGATAGATTTCCCAACTTAATGCCAACATACCATAAACCTCCC |
| | | ATTCTGCTAATGCCCAGCCTAAGTTGGGGAGACCACTCCAGATTCCAAG |
| | | ATGTACAGTTTGCTTTGCTGGGCCTTTTTCCCATGCCTGCCTTTACTCTG |
| | | CCAGAGTTATATTGCTGGGGTTTTGAAGAAGATCCTATTAAATAAAAGA |
| | | ATAAGCAGTATTATTAAGTAGCCCTGCATTTCAGGTTTCCTTGAGTGGC |
| | | AGGCCAGGCCTGGCCGTGAACGTTCACTGAAATCATGGCCTCTTGGCCA |
| | | AGATTGATAGCTTGTGCCTGTCCCTGAGTCCCAGTCCATCACGAGCAGC |
| | | TGGTTTCTAAGATGCTATTTCCCGTATAAAGCATGAGACCGTGACTTGC |
| | | CAGCCCCACAGAGCCCCGCCCTTGTCCATCACTGGCATCTGGACTCCAG |
| | | CCTGGGTTGGGGCAAAGAGGGAAATGAGATCATGTCCTAACCCTGATC |
| | | CTCTTGTCCCACAGATGAGGGCAGAGGCAGTCTGCTGACATGCGGTGAC |
| | | GTGGAAGAGAATCCCGGCCCTATGCTCAGGCTGCTCTTGGCTCTCAACT |
| | | TATTCCCTTCAATTCAAGTAACAGGAGAAGTCCAACTTCAACAGAGTGT |
| | | CGCGGATCTTGTTCGGCCAGGGGCCTCACTTAAATTGTCATGTACGGCT |
| | | TCTGGCTTTAACATAAAGTCCGCTTACATGCACTGGGTGATACAACGGC |
| | | CTGACCAATGTCCCGAGTGTCTTGGCAGAATTGATCCAGCCACCGGGAA |
| | | AACTAAGTACGCGCCGAAGTTTCAAGCAAAAGCGACCATAACCGCAGA |
| | | CACTTCCTCTAACACAGCCTACTTGCAACTGAGTTCACTCACGAGTGAG |
| | | GATACCGCTATTTATTATTGCACGCGAAGTTTGAATTGGGATTACGGTT |
| | | TGGATTACTGGGGTCAGGGCACTTCCGTTACCGTTTCCTCCGGCTCCAC |
| | | CTCTGGATCCGGCAAGCCCGGATCTGGCGAGGGATCCACCAAGGGCGA |
| | | CATCGTATTGACCCAGTCACCGGCAAGTTTGGCTGTGTCACTTGGGCAA |
| | | AGAGCCACAATCAGTTGCCGGGCATCAAAATCCGTTAGTATTCTGGGAA |
| | | CACATCTCATCCATTGGTATCAACAGAAGCCGGGCCAACCTCCGAAATT |
| | | GCTGATTTATGCTGCCAGCAATCTCGAATCTGGAGTCCCCGCGCGGTTC |
| | | AGCGGGAGTGGGTCTGAGACAGTGTTTACCTTGAACATTCATCCTGTAG |
| | | AAGAGGAAGACGCTGCGACATATTTCTGTCAACAGTCTATTGAAGACCC |
| | | TTGGACGTTTGGTTGCGGCACCAAGTTGGGCATCAAAGGTACCACCACA |
| | | ACGCCCGCTCCTCGGCCACCGACGCCAGCGCCAACTATTGCGAGTCAGC |
| | | CTCTCAGTCTGCGACCTGAGGCTTGTCGACCAGCAGCCGGAGGCGCAGT |
| | | GCACACGAGGGGGCTGGACTTCGCCTGTGATAGAAGACCTCCTTCTAAG |
| | | CCCTTTTGGGTGCTGGTGGTGGTTGGTGGAGTCCTGGCTTGCTATAGCTT |
| | | GCTAGTAACAGTGGCCTTTATTATTTTCTGGGTGAGGAAACGGGGCAGA |
| | | AAGAAACTCCTGTATATATTCAAACAACCATTTATGAGACCAGTACAAA |
| | | CTACTCAAGAGGAAGATGGCTGTAGCTGCCGATTTCCAGAAGAAGAAG |
| | | AAGGAGGATGTGAACTGGCTAGCCTGAGAGTGAAGTTCAGCAGGAGCG |
| | | CAGACGCCCCCGCGTACCAGCAGGGCCAGAACCAGCTCTATAACGAGC |
| | | TCAATCTAGGACGAAGAGAGGAGTACGATGTTTTGGACAAGAGACGTG |
| | | GCCGGGACCCTGAGATGGGGGGAAAGCCGCAGAGAAGGAAGAACCCT |
| | | CAGGAAGGCCTGTACAATGAACTGCAGAAAGATAAGATGGCGGAGGCC |
| | | TACAGTGAGATTGGGATGAAAGGCGAGCGCCGGAGGGGCAAGGGGCA |
| | | CGATGGCCTTTACCAGGGTCTCAGTACAGCCACCAAGGACACCTACGAC |
| | | GCCCTTCACATGCAGGCCCTGCCCCCTCGCGCTAGCGCCACGAACTTCT |
| | | CTCTGTTAAAGCAAGCAGGCGACGTGGAAGAAAACCCCGGTCCCGTGA |
| | | GCAAGGGCGAGGAGCTGTTCACCGGGGTGGTGCCCATCCTGGTCGAGC |
| | | TGGACGGCGACGTAAACGGCCACAAGTTCAGCGTGTCCGGCGAGGGCG |
| | | AGGGCGATGCCACCTACGGCAAGCTGACCCTGAAGTTCATCTGCACCAC |
| | | CGGCAAGCTGCCCGTGCCCTGGCCCACCCTCGTGACCACCCTGACCTAC |
| | | GGCGTGCAGTGCTTCAGCCGCTACCCCGACCACATGAAGCAGCACGAC |
| | | TTCTTCAAGTCCGCCATGCCCGAAGGCTACGTCCAGGAGCGCACCATCT |
| | | TCTTCAAGGACGACGGCAACTACAAGACCCGCGCCGAGGTGAAGTTCG |
| | | AGGGCGACACCCTGGTGAACCGCATCGAGCTGAAGGGCATCGACTTCA |
| | | AGGAGGACGGCAACATCCTGGGGCACAAGCTGGAGTACAACTACAACA |
| | | GCCACAACGTCTATATCATGGCCGACAAGCAGAAGAACGGCATCAAGG |
| | | TGAACTTCAAGATCCGCCACAACATCGAGGACGGCAGCGTGCAGCTCG |
| | | CCGACCACTACCAGCAGAACACCCCCATCGGCGACGGCCCCGTGCTGCT |
| | | GCCCGACAACCACTACCTGAGCACCCAGTCCGCCCTGAGCAAAGACCC |
| | | CAACGAGAAGCGCGATCACATGGTCCTGCTGGAGTTCGTGACCGCCGC |
| | | CGGGATCACTCTCGGCATGGACGAGCTGTACAAGTAATAAGCTTGGATC |
| | | TGCCTCGACTGTGCCTTCTAGTTGCCAGCCATCTGTTGTTTGCCCCTCCC |
| | | CCGTGCCTTCCTTGACCCTGGAAGGTGCCACTCCCACTGTCCTTTCCTAA |
| | | TAAAATGAGGAAATTGCATCGCATTGTCTGAGTAGGTGTCATTCTATTC |
| | | TGGGGGGTGGGGTGGGGCAGGACAGCAAGGGGGAGGATTGGGAAGAC |
| | | AATAGCAGGCATGCTGGGGAATCCAGAACCCTGACCCTGCCGTGTACC |
| | | AGCTGAGAGACTCTAAATCCAGTGACAAGTCTGTCTGCCTATTCACCGA |
| | | TTTTGATTCTCAAACAAATGTGTCACAAAGTAAGGATTCTGATGTGTAT |
| | | ATCACAGACAAAACTGTGCTAGACATGAGGTCTATGGACTTCAAGAGC |
| | | AACAGTGCTGTGGCCTGGAGCAACAAATCTGACTTTGCATGTGCAAACG |
| | | CCTTCAACAACAGCATTATTCCAGAAGACACCTTCTTCCCCAGCCCAGG |
| | | TAAGGGCAGCTTTGGTGCCTTCGCAGGCTGTTTCCTTGCTTCAGGAATG |
| | | GCCAGGTTCTGCCCAGAGCTCTGGTCAATGATGTCTAAAACTCCTCTGA |
| | | TTGGTGGTCTCGGCCTTATCCATTGCCACCAAAACCCTCTTTTTACTAAG |
| | | AAACAGTGAGCCTTGTTCTGGCAGTCCAGAGAATGACACGGGAAAAAA |
| | | GCAGATGAAGAGAAGGTGGCAGGAGAGGGCACGTGGCCCAGCCTCAGT |
| | | CTCTCCAACTGAGTTCCTGCCTGCCTGCCTTTGCTCAGACTGTTTGCCCC |
| | | TTACTGCTCTTCTAGGCCTCATTCTAAGCCCCTTCTCCAAGTTGCCTCTC |
| | | CTTATTTCTCCCTGTCTGCCAAAAAATCTTTCCCAGCTCACTAAGTCAGT |
| | | CTCACGCAGTCACTCATTAACCCACCAATCACTGATTGTGCCGGCACAT |
| | | GAATGCACCAGGTGTTGAAGTGGAGGAATTAAAAAGTCAGATGAGGGG |
| | | TGTGCCCAGAGGAAGCACCATTCTAGTTGGGGGAGCC |
| SEQ ID | FC sequence with | ASTKGPSVFPLAPSSKSTSGGTAALGCLVKDYFPEPVTVSWNSGALTSGVH |
| NO: 2 | ADCC enhancing | TFPAVLQSSGLYSLSSVVTVPSSSLGTQTYICNVNHKPSNTKVDKKVEPKSC |
| | mutations | DKTHTCPPCPAPELLGGPDVFLFPPKPKDTLMISRTPEVTCVVVDVSFEDPE |
| | | VKFNWYVDGVEVHNAKTKPREEQYNSTYRVVSVLTVLHQDWLNGKEYK |
| | | CKVTNKALPAPEEKTISKAKGQPREPQVYTLPPSRDELTKNQVSLTCLVKG |
| | | FYPSDIAVEWESNGQPENNYKTTPPVLDSDGSFFLYSKLTVDKSRWQQGN |
| | | VFSCSVMHEALHNHYTQKSLSLSPGK |
| SEQ ID | CD28 signal | MLRLLLALNLFPSIQVTG |
| NO: 3 | peptide | |
| SEQ ID | Linker | GSTSGSGKPGSGEGSTKG |
| NO: 4 | | |
| SEQ ID | CD8a hinge- | GTTTTPAPRPPTPAPTIASQPLSLRPEACRPAAGGAVHTRGLDFACDRRPPS |
| NO: 5 | CD28 | KPFWVLVVVGGVLACYSLLVTVAFIIFWVRKRGRKKLLYIFKQPFMRPVQ |
| | transmembrane-4- | TTQEEDGCSCRFPEEEEGGCELASLRVKFSRSADAPAYQQGQNQLYNELNL |
| | 1BB signal- | GRREEYDVLDKRRGRDPEMGGKPQRRKNPQEGLYNELQKDKMAEAYSEI |
| | CD247 signal | GMKGERRRGKGHDGLYQGLSTATKDTYDALHMQALPPRAS |
| SEQ ID | CD8a hinge | GTTTTPAPRPPTPAPTIASQPLSLRPEACRPAAGGAVHTRGLDFACDRRP |
| NO: 6 | | |
| SEQ ID | CD28 | PSKPFWVLVVVGGVLACYSLLVTVAFIIFWVR |
| NO: 7 | transmembrane | |
| SEQ ID | 4-1BB signal | KRGRKKLLYIFKQPFMRPVQTTQEEDGCSCRFPEEEEGGCELAS |
| NO: 8 | | |
| SEQ ID | CD247 signal | LRVKFSRSADAPAYQQGQNQLYNELNLGRREEYDVLDKRRGRDPEMGGK |
| NO: 9 | | PQRRKNPQEGLYNELQKDKMAEAYSEIGMKGERRRGKGHDGLYQGLSTA |
| | | TKDTYDALHMQALPPRAS |
| SEQ ID | VB2 VH CDR1 | GFNIKSA |
| NO: 10 | | |
| SEQ ID | VB2 VH CDR2 | DPATGK |
| NO: 11 | | |
| SEQ ID | VB2 VH CDR3 | SLNWDYGLDY |
| NO: 12 | | |
| SEQ ID | VB2 VL CDR1 | RASKSVSILGTHLIH |
| NO: 13 | | |
| SEQ ID | VB2 VL CDR2 | AASNLES |
| NO: 14 | | |
| SEQ ID | VB2 VL CDR3 | QQSIEDPWT |
| NO: 15 | | |
| SEQ ID | mCAR-Vb2 | MLRLLLALNLFPSIQVTGEVQLQQSVADLVRPGASLKLSCTASGFNIKSAY |
| NO: 16 | Complete CAR | MHWVIQRPDQCPECLGRIDPATGKTKYAPKFQAKATITADTSSNTAYLQLS |
| | | SLTSEDTAIYYCTRSLNWDYGLDYWGQGTSVTVSSGSTSGSGKPGSGEGST |
| | | KGDIVLTQSPASLAVSLGQRATISCRASKSVSILGTHLIHWYQQKPGQPPKL |
| | | LIYAASNLESGVPARFSGSGSETVFTLNIHPVEEEDAATYFCQQSIEDPWTF |
| | | GCGTKLGIKGTTTTPAPRPPTPAPTIASQPLSLRPEACRPAAGGAVHTRGLD |
| | | FACDRRPPSKPFWVLVVVGGVLACYSLLVTVAFIIFWVRKRGRKKLLYIFK |
| | | QPFMRPVQTTQEEDGCSCRFPEEEEGGCELASLRVKFSRSADAPAYQQGQN |
| | | QLYNELNLGRREEYDVLDKRRGRDPEMGGKPQRRKNPQEGLYNELQKDK |
| | | MAEAYSEIGMKGERRRGKGHDGLYQGLSTATKDTYDALHMQALPPRAS |
| SEQ ID | hCAR-Vb2-(V1- | MLRLLLALNLFPSIQVTGEVQLVQSGADLVRPGASLKLSCTASGFNIKSAY |
| NO: 17 | 46*01_1_V7- | MHWVRQAPGQCPEWLGRIDPATGKTKYAPKFQGRVTITADTSSNTAYLQL |
| | 3*01_1) | SSLTSEDTAIYYCTRSLNWDYGLDYWGQGTLVTVSSGSTSGSGKPGSGEGS |
| | Complete CAR | TKGDIVLTQSPASLAVSLGERATISCRASKSVSILGTHLIHWYQQKPGQPPK |
| | | LLIYAASNLESGVPDRFSGSGSGTDFTLTISRVEAEDVAVYYCQQSIEDPWT |
| | | FGCGTKLEIKGTTTTPAPRPPTPAPTIASQPLSLRPEACRPAAGGAVHTRGL |
| | | DFACDRRPPSKPFWVLVVVGGVLACYSLLVTVAFIIFWVRKRGRKKLLYIF |
| | | KQPFMRPVQTTQEEDGCSCRFPEEEEGGCELASLRVKFSRSADAPAYQQGQ |
| | | NQLYNELNLGRREEYDVLDKRRGRDPEMGGKPQRRKNPQEGLYNELQKD |
| | | KMAEAYSEIGMKGERRRGKGHDGLYQGLSTATKDTYDALHMQALPPRAS |
| SEQ ID | hCAR-Vb2-(V1- | MLRLLLALNLFPSIQVTGEVQLVQSGADLVRPGASLKLSCTASGFNIKSAY |
| NO: 18 | 46*01_1_V7- | MHWVRQAPGQCPEWLGRIDPATGKTKYAPKFQGRVTITADTSSNTAYLQL |
| | 3*01_2) | SSLTSEDTAIYYCTRSLNWDYGLDYWGQGTLVTVSSGSTSGSGKPGSGEGS |
| | Complete CAR | TKGDIVLTQSPASLAVSPGERATISCRASKSVSILGTHLIHWYQQKPGQPPK |
| | | LLIYAASNLESGVPDRFSGSGSGTDFTLTISRVEAEDVAVYYCQQSIEDPWT |
| | | FGCGTKVEIKGTTTTPAPRPPTPAPTIASQPLSLRPEACRPAAGGAVHTRGL |
| | | DFACDRRPPSKPFWVLVVVGGVLACYSLLVTVAFIIFWVRKRGRKKLLYIF |
| | | KQPFMRPVQTTQEEDGCSCRFPEEEEGGCELASLRVKFSRSADAPAYQQGQ |
| | | NQLYNELNLGRREEYDVLDKRRGRDPEMGGKPQRRKNPQEGLYNELQKD |
| | | KMAEAYSEIGMKGERRRGKGHDGLYQGLSTATKDTYDALHMQALPPRAS |
| SEQ ID | hCAR-Vb2-(V1- | MLRLLLALNLFPSIQVTGEVQLVQSGADLVRPGASLKLSCTASGFNIKSAY |
| NO: 19 | 46*01_1_V4- | MHWVRQAPGQCPEWLGRIDPATGKTKYAPKFQGRVTITADTSSNTAYLQL |
| | 1*01_1) | SSLTSEDTAIYYCTRSLNWDYGLDYWGQGTLVTVSSGSTSGSGKPGSGEGS |
| | Complete CAR | TKGDIVMTQSPDSLAVSLGERATINCRASKSVSILGTHLIHWYQQKPGQPPK |
| | | LLIYAASNLESGVPDRFSGSGSGTDFTLTISSLQAEDVAVYYCQQSIEDPWT |
| | | FGCGTKVEIKGTTTTPAPRPPTPAPTIASQPLSLRPEACRPAAGGAVHTRGL |
| | | DFACDRRPPSKPFWVLVVVGGVLACYSLLVTVAFIIFWVRKRGRKKLLYIF |
| | | KQPFMRPVQTTQEEDGCSCRFPEEEEGGCELASLRVKFSRSADAPAYQQGQ |
| | | NQLYNELNLGRREEYDVLDKRRGRDPEMGGKPQRRKNPQEGLYNELQKD |
| | | KMAEAYSEIGMKGERRRGKGHDGLYQGLSTATKDTYDALHMQALPPRAS |
| SEQ ID | hCAR-Vb2-(V1- | MLRLLLALNLFPSIQVTGEVQLVQSGADLVRPGASLKLSCTASGFNIKSAY |
| NO: 20 | 46*01_1_V3D- | MHWVRQAPGQCPEWLGRIDPATGKTKYAPKFQGRVTITADTSSNTAYLQL |
| | 20*02) Complete | SSLTSEDTAIYYCTRSLNWDYGLDYWGQGTLVTVSSGSTSGSGKPGSGEGS |
| | CAR | TKGEIVLTQSPATLSLSPGERATLSCRASKSVSILGTHLIHWYQQKPGQSPRL |
| | | LIYAASNLESGVPDRFSGSGSGTDFTLTISRLEAEDVAVYYCQQSIEDPWTF |
| | | GCGTKVEIKGTTTTPAPRPPTPAPTIASQPLSLRPEACRPAAGGAVHTRGLD |
| | | FACDRRPPSKPFWVLVVVGGVLACYSLLVTVAFIIFWVRKRGRKKLLYIFK |
| | | QPFMRPVQTTQEEDGCSCRFPEEEEGGCELASLRVKFSRSADAPAYQQGQN |
| | | QLYNELNLGRREEYDVLDKRRGRDPEMGGKPQRRKNPQEGLYNELQKDK |
| | | MAEAYSEIGMKGERRRGKGHDGLYQGLSTATKDTYDALHMQALPPRAS |
| SEQ ID | hCAR-Vb2-(V1- | MLRLLLALNLFPSIQVTGQVQLVQSGAEVKKPGASVKVSCKASGFNIKSAY |
| NO: 21 | 2*06_V7-3*01_1) | MHWVRQAPGQCLEWMGRIDPATGKTNYAQKFQGRVTMTRDTSISTAYM |
| | Complete CAR | ELSRLRSDDTAVYYCARSLNWDYGLDYWGQGTLVTVSSGSTSGSGKPGSG |
| | | EGSTKGDIVLTQSPASLAVSLGERATISCRASKSVSILGTHLIHWYQQKPGQ |
| | | PPKLLIYAASNLESGVPDRFSGSGSGTDFTLTISRVEAEDVAVYYCQQSIEDP |
| | | WTFGCGTKLEIKGTTTTPAPRPPTPAPTIASQPLSLRPEACRPAAGGAVHTR |
| | | GLDFACDRRPPSKPFWVLVVVGGVLACYSLLVTVAFIIFWVRKRGRKKLL |
| | | YIFKQPFMRPVQTTQEEDGCSCRFPEEEEGGCELASLRVKFSRSADAPAYQ |
| | | QGQNQLYNELNLGRREEYDVLDKRRGRDPEMGGKPQRRKNPQEGLYNEL |
| | | QKDKMAEAYSEIGMKGERRRGKGHDGLYQGLSTATKDTYDALHMQALPP |
| | | RAS |
| SEQ ID | hCAR-Vb2-(V1- | MLRLLLALNLFPSIQVTGQVQLVQSGAEVKKPGASVKVSCKASGFNIKSAY |
| NO: 22 | 2*06_V7-3*01_2) | MHWVRQAPGQCLEWMGRIDPATGKTNYAQKFQGRVTMTRDTSISTAYM |
| | Complete CAR | ELSRLRSDDTAVYYCARSLNWDYGLDYWGQGTLVTVSSGSTSGSGKPGSG |
| | | EGSTKGDIVLTQSPASLAVSPGERATISCRASKSVSILGTHLIHWYQQKPGQ |
| | | PPKLLIYAASNLESGVPDRFSGSGSGTDFTLTISRVEAEDVAVYYCQQSIEDP |
| | | WTFGCGTKVEIKGTTTTPAPRPPTPAPTIASQPLSLRPEACRPAAGGAVHTR |
| | | GLDFACDRRPPSKPFWVLVVVGGVLACYSLLVTVAFIIFWVRKRGRKKLL |
| | | YIFKQPFMRPVQTTQEEDGCSCRFPEEEEGGCELASLRVKFSRSADAPAYQ |
| | | QGQNQLYNELNLGRREEYDVLDKRRGRDPEMGGKPQRRKNPQEGLYNEL |
| | | QKDKMAEAYSEIGMKGERRRGKGHDGLYQGLSTATKDTYDALHMQALPP |
| | | RAS |
| SEQ ID | hCAR-Vb2-(V1- | MLRLLLALNLFPSIQVTGQVQLVQSGAEVKKPGASVKVSCKASGFNIKSAY |
| NO: 23 | 2*06_V4-1*01_1) | MHWVRQAPGQCLEWMGRIDPATGKTNYAQKFQGRVTMTRDTSISTAYM |
| | Complete CAR | ELSRLRSDDTAVYYCARSLNWDYGLDYWGQGTLVTVSSGSTSGSGKPGSG |
| | | EGSTKGDIVMTQSPDSLAVSLGERATINCRASKSVSILGTHLIHWYQQKPG |
| | | QPPKLLIYAASNLESGVPDRFSGSGSGTDFTLTISSLQAEDVAVYYCQQSIE |
| | | DPWTFGCGTKVEIKGTTTTPAPRPPTPAPTIASQPLSLRPEACRPAAGGAVH |
| | | TRGLDFACDRRPPSKPFWVLVVVGGVLACYSLLVTVAFIIFWVRKRGRKK |
| | | LLYIFKQPFMRPVQTTQEEDGCSCRFPEEEEGGCELASLRVKFSRSADAPAY |
| | | QQGQNQLYNELNLGRREEYDVLDKRRGRDPEMGGKPQRRKNPQEGLYNE |
| | | LQKDKMAEAYSEIGMKGERRRGKGHDGLYQGLSTATKDTYDALHMQAL |
| | | PPRAS |
| SEQ ID | hCAR-Vb2-(V1- | MLRLLLALNLFPSIQVTGQVQLVQSGAEVKKPGASVKVSCKASGFNIKSAY |
| NO: 24 | 2*06_V3D- | MHWVRQAPGQCLEWMGRIDPATGKTNYAQKFQGRVTMTRDTSISTAYM |
| | 20*02) Complete | ELSRLRSDDTAVYYCARSLNWDYGLDYWGQGTLVTVSSGSTSGSGKPGSG |
| | CAR | EGSTKGEIVLTQSPATLSLSPGERATLSCRASKSVSILGTHLIHWYQQKPGQ |
| | | SPRLLIYAASNLESGVPDRFSGSGSGTDFTLTISRLEAEDVAVYYCQQSIEDP |
| | | WTFGCGTKVEIKGTTTTPAPRPPTPAPTIASQPLSLRPEACRPAAGGAVHTR |
| | | GLDFACDRRPPSKPFWVLVVVGGVLACYSLLVTVAFIIFWVRKRGRKKLL |
| | | YIFKQPFMRPVQTTQEEDGCSCRFPEEEEGGCELASLRVKFSRSADAPAYQ |
| | | QGQNQLYNELNLGRREEYDVLDKRRGRDPEMGGKPQRRKNPQEGLYNEL |
| | | QKDKMAEAYSEIGMKGERRRGKGHDGLYQGLSTATKDTYDALHMQALPP |
| | | RAS |
| SEQ ID | hCAR-Vb2-(V1- | MLRLLLALNLFPSIQVTGQVQLVQSGAEVKKPGASVKVSCKASGFNIKSAA |
| NO: 25 | 3*01_V7-3*01_1) | MHWVRQAPGQCLEWMGWIDPATGKTKYSQKFQGRVTITRDTSASTAYME |
| | Complete CAR | LSSLRSEDTAVYYCARSLNWDYGLDYWGQGTLVTVSSGSTSGSGKPGSGE |
| | | GSTKGDIVLTQSPASLAVSLGERATISCRASKSVSILGTHLIHWYQQKPGQP |
| | | PKLLIYAASNLESGVPDRFSGSGSGTDFTLTISRVEAEDVAVYYCQQSIEDP |
| | | WTFGCGTKLEIKGTTTTPAPRPPTPAPTIASQPLSLRPEACRPAAGGAVHTR |
| | | GLDFACDRRPPSKPFWVLVVVGGVLACYSLLVTVAFIIFWVRKRGRKKLL |
| | | YIFKQPFMRPVQTTQEEDGCSCRFPEEEEGGCELASLRVKFSRSADAPAYQ |
| | | QGQNQLYNELNLGRREEYDVLDKRRGRDPEMGGKPQRRKNPQEGLYNEL |
| | | QKDKMAEAYSEIGMKGERRRGKGHDGLYQGLSTATKDTYDALHMQALPP |
| | | RAS |
| SEQ ID | hCAR-Vb2-(V1- | MLRLLLALNLFPSIQVTGQVQLVQSGAEVKKPGASVKVSCKASGFNIKSAA |
| NO: 26 | 3*01_V7-3*01_2) | MHWVRQAPGQCLEWMGWIDPATGKTKYSQKFQGRVTITRDTSASTAYME |
| | Complete CAR | LSSLRSEDTAVYYCARSLNWDYGLDYWGQGTLVTVSSGSTSGSGKPGSGE |
| | | GSTKGDIVLTQSPASLAVSPGERATISCRASKSVSILGTHLIHWYQQKPGQP |
| | | PKLLIYAASNLESGVPDRFSGSGSGTDFTLTISRVEAEDVAVYYCQQSIEDP |
| | | WTFGCGTKVEIKGTTTTPAPRPPTPAPTIASQPLSLRPEACRPAAGGAVHTR |
| | | GLDFACDRRPPSKPFWVLVVVGGVLACYSLLVTVAFIIFWVRKRGRKKLL |
| | | YIFKQPFMRPVQTTQEEDGCSCRFPEEEEGGCELASLRVKFSRSADAPAYQ |
| | | QGQNQLYNELNLGRREEYDVLDKRRGRDPEMGGKPQRRKNPQEGLYNEL |
| | | QKDKMAEAYSEIGMKGERRRGKGHDGLYQGLSTATKDTYDALHMQALPP |
| | | RAS |
| SEQ ID | hCAR-Vb2-(V1- | MLRLLLALNLFPSIQVTGQVQLVQSGAEVKKPGASVKVSCKASGFNIKSAA |
| NO: 27 | 3*01_V4-1*01_1) | MHWVRQAPGQCLEWMGWIDPATGKTKYSQKFQGRVTITRDTSASTAYME |
| | Complete CAR | LSSLRSEDTAVYYCARSLNWDYGLDYWGQGTLVTVSSGSTSGSGKPGSGE |
| | | GSTKGDIVMTQSPDSLAVSLGERATINCRASKSVSILGTHLIHWYQQKPGQP |
| | | PKLLIYAASNLESGVPDRFSGSGSGTDFTLTISSLQAEDVAVYYCQQSIEDP |
| | | WTFGCGTKVEIKGTTTTPAPRPPTPAPTIASQPLSLRPEACRPAAGGAVHTR |
| | | GLDFACDRRPPSKPFWVLVVVGGVLACYSLLVTVAFIIFWVRKRGRKKLL |
| | | YIFKQPFMRPVQTTQEEDGCSCRFPEEEEGGCELASLRVKFSRSADAPAYQ |
| | | QGQNQLYNELNLGRREEYDVLDKRRGRDPEMGGKPQRRKNPQEGLYNEL |
| | | QKDKMAEAYSEIGMKGERRRGKGHDGLYQGLSTATKDTYDALHMQALPP |
| | | RAS |
| SEQ ID | hCAR-Vb2-(V1- | MLRLLLALNLFPSIQVTGQVQLVQSGAEVKKPGASVKVSCKASGFNIKSAA |
| NO: 28 | 3*01_V3D- | MHWVRQAPGQCLEWMGWIDPATGKTKYSQKFQGRVTITRDTSASTAYME |
| | 20*02) Complete | LSSLRSEDTAVYYCARSLNWDYGLDYWGQGTLVTVSSGSTSGSGKPGSGE |
| | CAR | GSTKGEIVLTQSPATLSLSPGERATLSCRASKSVSILGTHLIHWYQQKPGQSP |
| | | RLLIYAASNLESGVPDRFSGSGSGTDFTLTISRLEAEDVAVYYCQQSIEDPW |
| | | TFGCGTKVEIKGTTTTPAPRPPTPAPTIASQPLSLRPEACRPAAGGAVHTRG |
| | | LDFACDRRPPSKPFWVLVVVGGVLACYSLLVTVAFIIFWVRKRGRKKLLYI |
| | | FKQPFMRPVQTTQEEDGCSCRFPEEEEGGCELASLRVKFSRSADAPAYQQG |
| | | QNQLYNELNLGRREEYDVLDKRRGRDPEMGGKPQRRKNPQEGLYNELQK |
| | | DKMAEAYSEIGMKGERRRGKGHDGLYQGLSTATKDTYDALHMQALPPRA |
| | | S |
| SEQ ID | hCAR-Vb2-(V1- | MLRLLLALNLFPSIQVTGQVQLVQSGAEVKKPGASVKVSCKASGFNIKSAY |
| NO: 29 | 46*01_2_V4- | MHWVRQAPGQCLEWLGRIDPATGKTKYAPKFQGRATITADTSSNTAYME |
| | 1*01_2) | LSSLRSEDTAVYYCTRSLNWDYGLDYWGQGTTVTVSSGSTSGSGKPGSGE |
| | Complete CAR | GSTKGDIVLTQSPDSLAVSLGERATINCKASKSVSILGTHLIHWYQQKPGQP |
| | | PKLLIYAASNLESGVPDRFSGSGSETVFTLTISSLQAEDVATYFCQQSIEDPW |
| | | TFGCGTKLEIKGTTTTPAPRPPTPAPTIASQPLSLRPEACRPAAGGAVHTRGL |
| | | DFACDRRPPSKPFWVLVVVGGVLACYSLLVTVAFIIFWVRKRGRKKLLYIF |
| | | KQPFMRPVQTTQEEDGCSCRFPEEEEGGCELASLRVKFSRSADAPAYQQGQ |
| | | NQLYNELNLGRREEYDVLDKRRGRDPEMGGKPQRRKNPQEGLYNELQKD |
| | | KMAEAYSEIGMKGERRRGKGHDGLYQGLSTATKDTYDALHMQALPPRAS |
| SEQ ID | hCAR-Vb2-(V1- | MLRLLLALNLFPSIQVTGQVQLVQSGAEVKKPGASVKVSCKASGFNIKSAY |
| NO: 30 | 46*01_2_V4- | MHWVRQAPGQCLEWLGRIDPATGKTKYAPKFQGRATITADTSSNTAYME |
| | 1*01_3) | LSSLRSEDTAVYYCTRSLNWDYGLDYWGQGTTVTVSSGSTSGSGKPGSGE |
| | Complete CAR | GSTKGDIVLTQSPDSLAVSLGERATINCKASKSVSILGTHLIHWYQQKPGQP |
| | | PKLLIYAASNRESGVPDRFSGSGSETDFTLTISSLQAEDVAVYYCQQSIEDP |
| | | WTFGCGTKLEIKGTTTTPAPRPPTPAPTIASQPLSLRPEACRPAAGGAVHTR |
| | | GLDFACDRRPPSKPFWVLVVVGGVLACYSLLVTVAFIIFWVRKRGRKKLL |
| | | YIFKQPFMRPVQTTQEEDGCCRFPEEEEGGCELASLRVKFSRSADAPAYQ |
| | | QGQNQLYNELNLGRREEYDVLDKRRGRDPEMGGKPQRRKNPQEGLYNEL |
| | | QKDKMAEAYSEIGMKGERRRGKGHDGLYQGLSTATKDTYDALHMQALPP |
| | | RAS |
| SEQ ID | hCAR-Vb2-(V1- | MLRLLLALNLFPSIQVTGQVQLVQSGAEVKKPGASVKVSCKASGFNIKSAY |
| NO: 31 | 46*01_2_V1- | MHWVRQAPGQCLEWLGRIDPATGKTKYAPKFQGRATITADTSSNTAYME |
| | 39*01_1) | LSSLRSEDTAVYYCTRSLNWDYGLDYWGQGTTVTVSSGSTSGSGKPGSGE |
| | Complete CAR | GSTKGDIQLTQSPSSLSASVGDRVTITCRASKSVSILGTHLIHWYQQKPGKA |
| | | PKLLIYAASNLESGVPSRFSGSGSETVFTLTISSLQPEDFATYFCQQSIEDPWT |
| | | FGCGTKLEIKGTTTTPAPRPPTPAPTIASQPLSLRPEACRPAAGGAVHTRGL |
| | | DFACDRRPPSKPFWVLVVVGGVLACYSLLVTVAFIIFWVRKRGRKKLLYIF |
| | | KQPFMRPVQTTQEEDGCSCRFPEEEEGGCELASLRVKFSRSADAPAYQQGQ |
| | | NQLYNELNLGRREEYDVLDKRRGRDPEMGGKPQRRKNPQEGLYNELQKD |
| | | KMAEAYSEIGMKGERRRGKGHDGLYQGLSTATKDTYDALHMQALPPRAS |
| SEQ ID | hCAR-Vb2-(V1- | MLRLLLALNLFPSIQVTGQVQLVQSGAEVKKPGASVKVSCKASGFNIKSAY |
| NO: 32 | 46*01_2_V1- | MHWVRQAPGQCLEWLGRIDPATGKTKYAPKFQGRATITADTSSNTAYME |
| | 39*01_2) | LSSLRSEDTAVYYCTRSLNWDYGLDYWGQGTTVTVSSGSTSGSGKPGSGE |
| | Complete CAR | GSTKGDIQLTQSPSSLSASVGDRVTITCRASKSVSILGTHLIHWYQQKPGKA |
| | | PKLLIYAASNLESGVPSRFSGSGSETDFTLTISSLQPEDFATYYCQQSIEDPW |
| | | TFGCGTKLEIKGTTTTPAPRPPTPAPTIASQPLSLRPEACRPAAGGAVHTRGL |
| | | DFACDRRPPSKPFWVLVVVGGVLACYSLLVTVAFIIFWVRKRGRKKLLYIF |
| | | KQPFMRPVQTTQEEDGCSCRFPEEEEGGCELASLRVKFSRSADAPAYQQGQ |
| | | NQLYNELNLGRREEYDVLDKRRGRDPEMGGKPQRRKNPQEGLYNELQKD |
| | | KMAEAYSEIGMKGERRRGKGHDGLYQGLSTATKDTYDALHMQALPPRAS |
| SEQ ID | hCAR-Vb2-(V1- | MLRLLLALNLFPSIQVTGQVQLVQSGAEVKKPGASVKVSCKASGFNIKSAY |
| NO: 33 | 46*01_3_V4- | MHWVRQAPGQCLEWMGRIDPATGKTKYAPKFQGRVTMTADTSTSTAYM |
| | 1*01_2) | ELSSLRSEDTAVYYCTRSLNWDYGLDYWGQGTTVTVSSGSTSGSGKPGSG |
| | Complete CAR | EGSTKGDIVLTQSPDSLAVSLGERATINCKASKSVSILGTHLIHWYQQKPGQ |
| | | PPKLLIYAASNLESGVPDRFSGSGSETVFTLTISSLQAEDVATYFCQQSIEDP |
| | | WTFGCGTKLEIKGTTTTPAPRPPTPAPTIASQPLSLRPEACRPAAGGAVHTR |
| | | GLDFACDRRPPSKPFWVLVVVGGVLACYSLLVTVAFIIFWVRKRGRKKLL |
| | | YIFKQPFMRPVQTTQEEDGCSCRFPEEEEGGCELASLRVKFSRSADAPAYQ |
| | | QGQNQLYNELNLGRREEYDVLDKRRGRDPEMGGKPQRRKNPQEGLYNEL |
| | | QKDKMAEAYSEIGMKGERRRGKGHDGLYQGLSTATKDTYDALHMQALPP |
| | | RAS |
| SEQ ID | hCAR-Vb2-(V1- | MLRLLLALNLFPSIQVTGQVQLVQSGAEVKKPGASVKVSCKASGFNIKSAY |
| NO: 34 | 46*01_3_V4- | MHWVRQAPGQCLEWMGRIDPATGKTKYAPKFQGRVTMTADTSTSTAYM |
| | 1*01_3) | ELSSLRSEDTAVYYCTRSLNWDYGLDYWGQGTTVTVSSGSTSGSGKPGSG |
| | Complete CAR | EGSTKGDIVLTQSPDSLAVSLGERATINCKASKSVSILGTHLIHWYQQKPGQ |
| | | PPKLLIYAASNRESGVPDRFSGSGSETDFTLTISSLQAEDVAVYYCQQSIEDP |
| | | WTFGCGTKLEIKGTTTTPAPRPPTPAPTIASQPLSLRPEACRPAAGGAVHTR |
| | | GLDFACDRRPPSKPFWVLVVVGGVLACYSLLVTVAFIIFWVRKRGRKKLL |
| | | YIFKQPFMRPVQTTQEEDGCSCRFPEEEEGGCELASLRVKFSRSADAPAYQ |
| | | QGQNQLYNELNLGRREEYDVLDKRRGRDPEMGGKPQRRKNPQEGLYNEL |
| | | QKDKMAEAYSEIGMKGERRRGKGHDGLYQGLSTATKDTYDALHMQALPP |
| | | RAS |
| SEQ ID | hCAR-Vb2-(V1- | MLRLLLALNLFPSIQVTGQVQLVQSGAEVKKPGASVKVSCKASGFNIKSAY |
| NO: 35 | 46*01_3_V1- | MHWVRQAPGQCLEWMGRIDPATGKTKYAPKFQGRVTMTADTSTSTAYM |
| | 39*01_1) | ELSSLRSEDTAVYYCTRSLNWDYGLDYWGQGTTVTVSSGSTSGSGKPGSG |
| | Complete CAR | EGSTKGDIQLTQSPSSLSASVGDRVTITCRASKSVSILGTHLIHWYQQKPGK |
| | | APKLLIYAASNLESGVPSRFSGSGSETVFTLTISSLQPEDFATYFCQQSIEDP |
| | | WTFGCGTKLEIKGTTTTPAPRPPTPAPTIASQPLSLRPEACRPAAGGAVHTR |
| | | GLDFACDRRPPSKPFWVLVVVGGVLACYSLLVTVAFIIFWVRKRGRKKLL |
| | | YIFKQPFMRPVQTTQEEDGCSCRFPEEEEGGCELASLRVKFSRSADAPAYQ |
| | | QGQNQLYNELNLGRREEYDVLDKRRGRDPEMGGKPQRRKNPQEGLYNEL |
| | | QKDKMAEAYSEIGMKGERRRGKGHDGLYQGLSTATKDTYDALHMQALPP |
| | | RAS |
| SEQ ID | hCAR-Vb2-(V1- | MLRLLLALNLFPSIQVTGQVQLVQSGAEVKKPGASVKVSCKASGFNIKSAY |
| NO: 36 | 46*01_3_V1- | MHWVRQAPGQCLEWMGRIDPATGKTKYAPKFQGRVTMTADTSTSTAYM |
| | 39*01_2) | ELSSLRSEDTAVYYCTRSLNWDYGLDYWGQGTTVTVSSGSTSGSGKPGSG |
| | Complete CAR | EGSTKGDIQLTQSPSSLSASVGDRVTITCRASKSVSILGTHLIHWYQQKPGK |
| | | APKLLIYAASNLESGVPSRFSGSGSETDFTLTISSLQPEDFATYYCQQSIEDP |
| | | WTFGCGTKLEIKGTTTTPAPRPPTPAPTIASQPLSLRPEACRPAAGGAVHTR |
| | | GLDFACDRRPPSKPFWVLVVVGGVLACYSLLVTVAFIIFWVRKRGRKKLL |
| | | YIFKQPFMRPVQTTQEEDGCSCRFPEEEEGGCELASLRVKFSRSADAPAYQ |
| | | QGQNQLYNELNLGRREEYDVLDKRRGRDPEMGGKPQRRKNPQEGLYNEL |
| | | QKDKMAEAYSEIGMKGERRRGKGHDGLYQGLSTATKDTYDALHMQALPP |
| | | RAS |
| SEQ ID | hCAR-Vb2-(V7- | MLRLLLALNLFPSIQVTGQVQLQQSGPGLVKPSETLSLTCTASGFNIKSAYM |
| NO: 37 | 4-1*02_1_V4- | HWVRQPPGKCLEWLGRIDPATGKTKYAPSLKSRATISADTSSNTASLKLSS |
| | 1*01_2) | VTAADTAVYYCTRSLNWDYGLDYWGQGTTVTVSSGSTSGSGKPGSGEGS |
| | Complete CAR | TKGDIVLTQSPDSLAVSLGERATINCKASKSVSILGTHLIHWYQQKPGQPPK |
| | | LLIYAASNLESGVPDRFSGSGSETVFTLTISSLQAEDVATYFCQQSIEDPWTF |
| | | GCGTKLEIKGTTTTPAPRPPTPAPTIASQPLSLRPEACRPAAGGAVHTRGLD |
| | | FACDRRPPSKPFWVLVVVGGVLACYSLLVTVAFIIFWVRKRGRKKLLYIFK |
| | | QPFMRPVQTTQEEDGCSCRFPEEEEGGCELASLRVKFSRSADAPAYQQGQN |
| | | QLYNELNLGRREEYDVLDKRRGRDPEMGGKPQRRKNPQEGLYNELQKDK |
| | | MAEAYSEIGMKGERRRGKGHDGLYQGLSTATKDTYDALHMQALPPRAS |
| SEQ ID | hCAR-Vb2-(V7- | MLRLLLALNLFPSIQVTGQVQLQQSGPGLVKPSETLSLTCTASGFNIKSAYM |
| NO: 38 | 4-1*02_1_V4- | HWVRQPPGKCLEWLGRIDPATGKTKYAPSLKSRATISADTSSNTASLKLSS |
| | 1*01_3) | VTAADTAVYYCTRSLNWDYGLDYWGQGTTVTVSSGSTSGSGKPGSGEGS |
| | Complete CAR | TKGDIVLTQSPDSLAVSLGERATINCKASKSVSILGTHLIHWYQQKPGQPPK |
| | | LLIYAASNRESGVPDRFSGSGSETDFTLTISSLQAEDVAVYYCQQSIEDPWT |
| | | FGCGTKLEIKGTTTTPAPRPPTPAPTIASQPLSLRPEACRPAAGGAVHTRGL |
| | | DFACDRRPPSKPFWVLVVVGGVLACYSLLVTVAFIIFWVRKRGRKKLLYIF |
| | | KQPFMRPVQTTQEEDGCSCRFPEEEEGGCELASLRVKFSRSADAPAYQQGQ |
| | | NQLYNELNLGRREEYDVLDKRRGRDPEMGGKPQRRKNPQEGLYNELQKD |
| | | KMAEAYSEIGMKGERRRGKGHDGLYQGLSTATKDTYDALHMQALPPRAS |
| SEQ ID | hCAR-Vb2-(V7- | MLRLLLALNLFPSIQVTGQVQLQQSGPGLVKPSETLSLTCTASGFNIKSAYM |
| NO: 39 | 4-1*02_1_V1- | HWVRQPPGKCLEWLGRIDPATGKTKYAPSLKSRATISADTSSNTASLKLSS |
| | 39*01_1) | VTAADTAVYYCTRSLNWDYGLDYWGQGTTVTVSSGSTSGSGKPGSGEGS |
| | Complete CAR | TKGDIQLTQSPSSLSASVGDRVTITCRASKSVSILGTHLIHWYQQKPGKAPK |
| | | LLIYAASNLESGVPSRFSGSGSETVFTLTISSLQPEDFATYFCQQSIEDPWTFG |
| | | CGTKLEIKGTTTTPAPRPPTPAPTIASQPLSLRPEACRPAAGGAVHTRGLDF |
| | | ACDRRPPSKPFWVLVVVGGVLACYSLLVTVAFIIFWVRKRGRKKLLYIFKQ |
| | | PFMRPVQTTQEEDGCSCRFPEEEEGGCELASLRVKFSRSADAPAYQQGQNQ |
| | | LYNELNLGRREEYDVLDKRRGRDPEMGGKPQRRKNPQEGLYNELQKDKM |
| | | AEAYSEIGMKGERRRGKGHDGLYQGLSTATKDTYDALHMQALPPRAS |
| SEQ ID | hCAR-Vb2-(V7- | MLRLLLALNLFPSIQVTGQVQLQQSGPGLVKPSETLSLTCTASGFNIKSAYM |
| NO: 40 | 4-1*02_1_V1- | HWVRQPPGKCLEWLGRIDPATGKTKYAPSLKSRATISADTSSNTASLKLSS |
| | 39*01_2) | VTAADTAVYYCTRSLNWDYGLDYWGQGTTVTVSSGSTSGSGKPGSGEGS |
| | Complete CAR | TKGDIQLTQSPSSLSASVGDRVTITCRASKSVSILGTHLIHWYQQKPGKAPK |
| | | LLIYAASNLESGVPSRFSGSGSETDFTLTISSLQPEDFATYYCQQSIEDPWTF |
| | | GCGTKLEIKGTTTTPAPRPPTPAPTIASQPLSLRPEACRPAAGGAVHTRGLD |
| | | FACDRRPPSKPFWVLVVVGGVLACYSLLVTVAFIIFWVRKRGRKKLLYIFK |
| | | QPFMRPVQTTQEEDGCSCRFPEEEEGGCELASLRVKFSRSADAPAYQQGQN |
| | | QLYNELNLGRREEYDVLDKRRGRDPEMGGKPQRRKNPQEGLYNELQKDK |
| | | MAEAYSEIGMKGERRRGKGHDGLYQGLSTATKDTYDALHMQALPPRAS |
| SEQ ID | hCAR-Vb2-(V7- | MLRLLLALNLFPSIQVTGQVQLQESGPGLVKPSETLSLTCTASGFNIKSAYM |
| NO: 41 | 4-1*02_2_V4- | HWIRQPPGKCLEWIGRIDPATGKTKYAPSLKSRVTISADTSKNQASLKLSSV |
| | 1*01_2) | TAADTAVYYCTRSLNWDYGLDYWGQGTTVTVSSGSTSGSGKPGSGEGST |
| | Complete CAR | KGDIVLTQSPDSLAVSLGERATINCKASKSVSILGTHLIHWYQQKPGQPPKL |
| | | LIYAASNLESGVPDRFSGSGSETVFTLTISSLQAEDVATYFCQQSIEDPWTFG |
| | | CGTKLEIKGTTTTPAPRPPTPAPTIASQPLSLRPEACRPAAGGAVHTRGLDF |
| | | ACDRRPPSKPFWVLVVVGGVLACYSLLVTVAFIIFWVRKRGRKKLLYIFKQ |
| | | PFMRPVQTTQEEDGCSCRFPEEEEGGCELASLRVKFSRSADAPAYQQGQNQ |
| | | LYNELNLGRREEYDVLDKRRGRDPEMGGKPQRRKNPQEGLYNELQKDKM |
| | | AEAYSEIGMKGERRRGKGHDGLYQGLSTATKDTYDALHMQALPPRAS |
| SEQ ID | hCAR-Vb2-(V7- | MLRLLLALNLFPSIQVTGQVQLQESGPGLVKPSETLSLTCTASGFNIKSAYM |
| NO: 42 | 4-1*02_2_V4- | HWIRQPPGKCLEWIGRIDPATGKTKYAPSLKSRVTISADTSKNQASLKLSSV |
| | 1*01_3) | TAADTAVYYCTRSLNWDYGLDYWGQGTTVTVSSGSTSGSGKPGSGEGST |
| | Complete CAR | KGDIVLTQSPDSLAVSLGERATINCKASKSVSILGTHLIHWYQQKPGQPPKL |
| | | LIYAASNRESGVPDRFSGSGSETDFTLTISSLQAEDVAVYYCQQSIEDPWTF |
| | | GCGTKLEIKGTTTTPAPRPPTPAPTIASQPLSLRPEACRPAAGGAVHTRGLD |
| | | FACDRRPPSKPFWVLVVVGGVLACYSLLVTVAFIIFWVRKRGRKKLLYIFK |
| | | QPFMRPVQTTQEEDGCSCRFPEEEEGGCELASLRVKFSRSADAPAYQQGQN |
| | | QLYNELNLGRREEYDVLDKRRGRDPEMGGKPQRRKNPQEGLYNELQKDK |
| | | MAEAYSEIGMKGERRRGKGHDGLYQGLSTATKDTYDALHMQALPPRAS |
| SEQ ID | hCAR-Vb2-(V7- | MLRLLLALNLFPSIQVTGQVQLQESGPGLVKPSETLSLTCTASGFNIKSAYM |
| NO: 43 | 4-1*02_2_V1- | HWIRQPPGKCLEWIGRIDPATGKTKYAPSLKSRVTISADTSKNQASLKLSSV |
| | 39*01_1) | TAADTAVYYCTRSLNWDYGLDYWGQGTTVTVSSGSTSGSGKPGSGEGST |
| | Complete CAR | KGDIQLTQSPSSLSASVGDRVTITCRASKSVSILGTHLIHWYQQKPGKAPKL |
| | | LIYAASNLESGVPSRFSGSGSETVFTLTISSLQPEDFATYFCQQSIEDPWTFG |
| | | CGTKLEIKGTTTTPAPRPPTPAPTIASQPLSLRPEACRPAAGGAVHTRGLDF |
| | | ACDRRPPSKPFWVLVVVGGVLACYSLLVTVAFIIFWVRKRGRKKLLYIFKQ |
| | | PFMRPVQTTQEEDGCSCRFPEEEEGGCELASLRVKFSRSADAPAYQQGQNQ |
| | | LYNELNLGRREEYDVLDKRRGRDPEMGGKPQRRKNPQEGLYNELQKDKM |
| | | AEAYSEIGMKGERRRGKGHDGLYQGLSTATKDTYDALHMQALPPRAS |
| SEQ ID | hCAR-Vb2-(V7- | MLRLLLALNLFPSIQVTGQVQLQESGPGLVKPSETLSLTCTASGFNIKSAYM |
| NO: 44 | 4-1*02_2_V1- | HWIRQPPGKCLEWIGRIDPATGKTKYAPSLKSRVTISADTSKNQASLKLSSV |
| | 39*01_2) | TAADTAVYYCTRSLNWDYGLDYWGQGTTVTVSSGSTSGSGKPGSGEGST |
| | Complete CAR | KGDIQLTQSPSSLSASVGDRVTITCRASKSVSILGTHLIHWYQQKPGKAPKL |
| | | LIYAASNLESGVPSRFSGSGSETDFTLTISSLQPEDFATYYCQQSIEDPWTFG |
| | | CGTKLEIKGTTTTPAPRPPTPAPTIASQPLSLRPEACRPAAGGAVHTRGLDF |
| | | ACDRRPPSKPFWVLVVVGGVLACYSLLVTVAFIIFWVRKRGRKKLLYIFKQ |
| | | PFMRPVQTTQEEDGCSCRFPEEEEGGCELASLRVKFSRSADAPAYQQGQNQ |
| | | LYNELNLGRREEYDVLDKRRGRDPEMGGKPQRRKNPQEGLYNELQKDKM |
| | | AEAYSEIGMKGERRRGKGHDGLYQGLSTATKDTYDALHMQALPPRAS |
| SEQ ID | hCAR-Vb2-(V1- | EVQLVQSGADLVRPGASLKLSCTASGFNIKSAYMHWVRQAPGQCPEWLG |
| NO: 45 | 46*01_1_V7- | RIDPATGKTKYAPKFQGRVTITADTSSNTAYLQLSSLTSEDTAIYYCTRSLN |
| | 3*01_1) VH | WDYGLDYWGQGTLVTVSS |
| SEQ ID | hCAR-Vb2-(V1- | EVQLVQSGADLVRPGASLKLSCTASGFNIKSAYMHWVRQAPGQCPEWLG |
| NO: 46 | 46*01_1_V7- | RIDPATGKTKYAPKFQGRVTITADTSSNTAYLQLSSLTSEDTAIYYCTRSLN |
| | 3*01_2) VH | WDYGLDYWGQGTLVTVSS |
| SEQ ID | hCAR-Vb2-(V1- | EVQLVQSGADLVRPGASLKLSCTASGFNIKSAYMHWVRQAPGQCPEWLG |
| NO: 47 | 46*01_1_V4- | RIDPATGKTKYAPKFQGRVTITADTSSNTAYLQLSSLTSEDTAIYYCTRSLN |
| | 1*01_1) VH | WDYGLDYWGQGTLVTVSS |
| SEQ ID | hCAR-Vb2-(V1- | EVQLVQSGADLVRPGASLKLSCTASGFNIKSAYMHWVRQAPGQCPEWLG |
| NO: 48 | 46*01_1_V3D- | RIDPATGKTKYAPKFQGRVTITADTSSNTAYLQLSSLTSEDTAIYYCTRSLN |
| | 20*02) VH | WDYGLDYWGQGTLVTVSS |
| SEQ ID | hCAR-Vb2-(V1- | QVQLVQSGAEVKKPGASVKVSCKASGFNIKSAYMHWVRQAPGQCLEWM |
| NO: 49 | 2*06_V7-3*01_1) | GRIDPATGKTNYAQKFQGRVTMTRDTSISTAYMELSRLRSDDTAVYYCAR |
| | VH | SLNWDYGLDYWGQGTLVTVSS |
| SEQ ID | hCAR-Vb2-(V1- | QVQLVQSGAEVKKPGASVKVSCKASGFNIKSAYMHWVRQAPGQCLEWM |
| NO: 50 | 2*06_V7-3*01_2) | GRIDPATGKTNYAQKFQGRVTMTRDTSISTAYMELSRLRSDDTAVYYCAR |
| | VH | SLNWDYGLDYWGQGTLVTVSS |
| SEQ ID | hCAR-Vb2-(V1- | QVQLVQSGAEVKKPGASVKVSCKASGFNIKSAYMHWVRQAPGQCLEWM |
| NO: 51 | 2*06_V4-1*01_1) | GRIDPATGKTNYAQKFQGRVTMTRDTSISTAYMELSRLRSDDTAVYYCAR |
| | VH | SLNWDYGLDYWGQGTLVTVSS |
| SEQ ID | hCAR-Vb2-(V1- | QVQLVQSGAEVKKPGASVKVSCKASGFNIKSAYMHWVRQAPGQCLEWM |
| NO: 52 | 2*06_V3D- | GRIDPATGKTNYAQKFQGRVTMTRDTSISTAYMELSRLRSDDTAVYYCAR |
| | 20*02) VH | SLNWDYGLDYWGQGTLVTVSS |
| SEQ ID | hCAR-Vb2-(V1- | QVQLVQSGAEVKKPGASVKVSCKASGFNIKSAAMHWVRQAPGQCLEWM |
| NO: 53 | 3*01_V7-3*01_1) | GWIDPATGKTKYSQKFQGRVTITRDTSASTAYMELSSLRSEDTAVYYCARS |
| | VH | LNWDYGLDYWGQGTLVTVSS |
| SEQ ID | hCAR-Vb2-(V1- | QVQLVQSGAEVKKPGASVKVSCKASGFNIKSAAMHWVRQAPGQCLEWM |
| NO: 54 | 3*01_V7-3*01_2) | GWIDPATGKTKYSQKFQGRVTITRDTSASTAYMELSSLRSEDTAVYYCARS |
| | VH | LNWDYGLDYWGQGTLVTVSS |
| SEQ ID | hCAR-Vb2-(V1- | QVQLVQSGAEVKKPGASVKVSCKASGFNIKSAAMHWVRQAPGQCLEWM |
| NO: 55 | 3*01_V4-1*01_1) | GWIDPATGKTKYSQKFQGRVTITRDTSASTAYMELSSLRSEDTAVYYCARS |
| | VH | LNWDYGLDYWGQGTLVTVSS |
| SEQ ID | hCAR-Vb2-(V1- | QVQLVQSGAEVKKPGASVKVSCKASGFNIKSAAMHWVRQAPGQCLEWM |
| NO: 56 | 3*01_V3D- | GWIDPATGKTKYSQKFQGRVTITRDTSASTAYMELSSLRSEDTAVYYCARS |
| | 20*02) VH | LNWDYGLDYWGQGTLVTVSS |
| SEQ ID | hCAR-Vb2-(V1- | QVQLVQSGAEVKKPGASVKVSCKASGFNIKSAYMHWVRQAPGQCLEWLG |
| NO: 57 | 46*01_2_V4- | RIDPATGKTKYAPKFQGRATITADTSSNTAYMELSSLRSEDTAVYYCTRSL |
| | 1*01_2) VH | NWDYGLDYWGQGTTVTVSS |
| SEQ ID | hCAR-Vb2-(V1- | QVQLVQSGAEVKKPGASVKVSCKASGFNIKSAYMHWVRQAPGQCLEWLG |
| NO: 58 | 46*01_2_V4- | RIDPATGKTKYAPKFQGRATITADTSSNTAYMELSSLRSEDTAVYYCTRSL |
| | 1*01_3) VH | NWDYGLDYWGQGTTVTVSS |
| SEQ ID | hCAR-Vb2-(V1- | QVQLVQSGAEVKKPGASVKVSCKASGFNIKSAYMHWVRQAPGQCLEWLG |
| NO: 59 | 46*01_2_V1- | RIDPATGKTKYAPKFQGRATITADTSSNTAYMELSSLRSEDTAVYYCTRSL |
| | 39*01_1) VH | NWDYGLDYWGQGTTVTVSS |
| SEQ ID | hCAR-Vb2-(V1- | QVQLVQSGAEVKKPGASVKVSCKASGFNIKSAYMHWVRQAPGQCLEWLG |
| NO: 60 | 46*01_2_V1- | RIDPATGKTKYAPKFQGRATITADTSSNTAYMELSSLRSEDTAVYYCTRSL |
| | 39*01_2) VH | NWDYGLDYWGQGTTVTVSS |
| SEQ ID | hCAR-Vb2-(V1- | QVQLVQSGAEVKKPGASVKVSCKASGFNIKSAYMHWVRQAPGQCLEWM |
| NO: 61 | 46*01_3_V4- | GRIDPATGKTKYAPKFQGRVTMTADTSTSTAYMELSSLRSEDTAVYYCTRS |
| | 1*01_2) VH | LNWDYGLDYWGQGTTVTVSS |
| SEQ ID | hCAR-Vb2-(V1- | QVQLVQSGAEVKKPGASVKVSCKASGFNIKSAYMHWVRQAPGQCLEWM |
| NO: 62 | 46*01_3_V4- | GRIDPATGKTKYAPKFQGRVTMTADTSTSTAYMELSSLRSEDTAVYYCTRS |
| | 1*01_3) VH | LNWDYGLDYWGQGTTVTVSS |
| SEQ ID | hCAR-Vb2-(V1- | QVQLVQSGAEVKKPGASVKVSCKASGFNIKSAYMHWVRQAPGQCLEWM |
| NO: 63 | 46*01_3_V1- | GRIDPATGKTKYAPKFQGRVTMTADTSTSTAYMELSSLRSEDTAVYYCTRS |
| | 39*01_1) VH | LNWDYGLDYWGQGTTVTVSS |
| SEQ ID | hCAR-Vb2-(V1- | QVQLVQSGAEVKKPGASVKVSCKASGFNIKSAYMHWVRQAPGQCLEWM |
| NO: 64 | 46*01_3_V1- | GRIDPATGKTKYAPKFQGRVTMTADTSTSTAYMELSSLRSEDTAVYYCTRS |
| | 39*01_2) VH | LNWDYGLDYWGQGTTVTVSS |
| SEQ ID | hCAR-Vb2-(V7- | QVQLQQSGPGLVKPSETLSLTCTASGFNIKSAYMHWVRQPPGKCLEWLGRI |
| NO: 65 | 4-1*02_1_V4- | DPATGKTKYAPSLKSRATISADTSSNTASLKLSSVTAADTAVYYCTRSLNW |
| | 1*01_2) VH | DYGLDYWGQGTTVTVSS |
| SEQ ID | hCAR-Vb2-(V7- | QVQLQQSGPGLVKPSETLSLTCTASGFNIKSAYMHWVRQPPGKCLEWLGRI |
| NO: 66 | 4-1*02_1_V4- | DPATGKTKYAPSLKSRATISADTSSNTASLKLSSVTAADTAVYYCTRSLNW |
| | 1*01_3) VH | DYGLDYWGQGTTVTVSS |
| SEQ ID | hCAR-Vb2-(V7- | QVQLQQSGPGLVKPSETLSLTCTASGFNIKSAYMHWVRQPPGKCLEWLGRI |
| NO: 67 | 4-1*02_1_V1- | DPATGKTKYAPSLKSRATISADTSSNTASLKLSSVTAADTAVYYCTRSLNW |
| | 39*01_1) VH | DYGLDYWGQGTTVTVSS |
| SEQ ID | hCAR-Vb2-(V7- | QVQLQQSGPGLVKPSETLSLTCTASGFNIKSAYMHWVRQPPGKCLEWLGRI |
| NO: 68 | 4-1*02_1_V1- | DPATGKTKYAPSLKSRATISADTSSNTASLKLSSVTAADTAVYYCTRSLNW |
| | 39*01_2) VH | DYGLDYWGQGTTVTVSS |
| SEQ ID | hCAR-Vb2-(V7- | QVQLQESGPGLVKPSETLSLTCTASGFNIKSAYMHWIRQPPGKCLEWIGRID |
| NO: 69 | 4-1*02_2_V4- | PATGKTKYAPSLKSRVTISADTSKNQASLKLSSVTAADTAVYYCTRSLNW |
| | 1*01_2) VH | DYGLDYWGQGTTVTVSS |
| SEQ ID | hCAR-Vb2-(V7- | QVQLQESGPGLVKPSETLSLTCTASGFNIKSAYMHWIRQPPGKCLEWIGRID |
| NO: 70 | 4-1*02_2_V4- | PATGKTKYAPSLKSRVTISADTSKNQASLKLSSVTAADTAVYYCTRSLNW |
| | 1*01_3) VH | DYGLDYWGQGTTVTVSS |
| SEQ ID | hCAR-Vb2-(V7- | QVQLQESGPGLVKPSETLSLTCTASGFNIKSAYMHWIRQPPGKCLEWIGRID |
| NO: 71 | 4-1*02_2_V1- | PATGKTKYAPSLKSRVTISADTSKNQASLKLSSVTAADTAVYYCTRSLNW |
| | 39*01_1) VH | DYGLDYWGQGTTVTVSS |
| SEQ ID | hCAR-Vb2-(V7- | QVQLQESGPGLVKPSETLSLTCTASGFNIKSAYMHWIRQPPGKCLEWIGRID |
| NO: 72 | 4-1*02_2_V1- | PATGKTKYAPSLKSRVTISADTSKNQASLKLSSVTAADTAVYYCTRSLNW |
| | 39*01_2) VH | DYGLDYWGQGTTVTVSS |
| SEQ ID | VB 7.2 | QVHLVQSGAEVKRPGASVKVSCKASGYIFTDYYMHWVRQAPGQCLEWM |
| NO: 73 | Humanized VH 1 | GWINPSSGYTTYAQKFQDRVTLTADKSSSTAYMELSSLTSEDTAVYYCARS |
| | | LQLGRDYWGQGTLVTVSS |
| SEQ ID | VB 7.2 | QVQLVQSGAEVKKPGASVKVSCKASGYIFTDYYMHWVRQAPGQCLEWM |
| NO: 74 | Humanized VH 2 | GWINPSSGYTNYAQKFQGRVTMTRDTSISTAYMELSRLRSDDTAVYYCAR |
| | | SLQLGRDYWGQGTLVTVSS |
| SEQ ID | VB 7.2 | QVQLVQSGAEVKKPGASVKVSCKASGYIFTDYYMHWVRQAPGQCLEWM |
| NO: 75 | Humanized VH 3 | GIINPSSGYTSYAQKFQGRVTMTRDTSTSTVYMELSSLRSEDTAVYYCARS |
| | | LQLGRDYWGQGTLVTVSS |
| SEQ ID | VB 4 Humanized | QVQLVQSGAEVKKPGASVKVSCKASGYTFTSYYIYWVRQAPGQCLEWLG |
| NO: 76 | VH 1 | WIYPGNGGTYYSEKFQGRVTFTADTSTNTAYMELSSLTSEDTAVYFCARGS |
| | | GDRYNSLAYWGQGTLVTVSS |
| SEQ ID | VB 4 Humanized | QVQLVQSGAEVKKPGASVKVSCKASGYTFTSYAMHWVRQAPGQCLEWM |
| NO: 77 | VH 2 | GWIYPGNGGTKYSQKFQGRVTITRDTSASTAYMELSSLRSEDTAVYYCAR |
| | | GSGDRYNSLAYWGQGTLVTVSS |
| SEQ ID | VB 4 Humanized | QVQLVQSGAEVKKPGASVKVSCKASGYTFTSYYMHWVRQAPGQCLEWM |
| NO: 78 | VH 3 | GIIYPGNGGTSYAQKFQGRVTMTRDTSTSTVYMELSSLRSEDTAVYYCARG |
| | | SGDRYNSLAYWGQGTLVTVSS |
| SEQ ID | VB 4 Humanized | QVQLVQSGAEVKKPGASVKVSCKASGYTFTSYGISWVRQAPGQCLEWMG |
| NO: 79 | VH 4 | WIYPGNGGTNYAQKLQGRVTMTTDTSTSTAYMELRSLRSDDTAVYYCAR |
| | | GSGDRYNSLAYWGQGTLVTVSS |
| SEQ ID | VB 4 Humanized | QVQLVQSGAEVKKPGASVKVSCKASGYTFTSYYMHWVRQAPGQCLEWM |
| NO: 80 | VH 5 | GWIYPGNGGTNYAQKFQGRVTMTRDTSISTAYMELSRLRSDDTAVYYCAR |
| | | GSGDRYNSLAYWGQGTLVTVSS |
| SEQ ID | VB 13.2 | QVQLVQSGPEVKKPGASVKVSCKASGYTFTDYYIHWLRQAPGKCLEWIG |
| NO: 81 | Humanized VH 1 | WVNTNNGDTNYAQRFKGRVSLTRDKSTSTAYMELNSLTSEDTAVYYCAR |
| | | ALYTGSYWFAYWGQGTLVTVSS |
| SEQ ID | VB 13.2 | QVQLVQSGAEVKKPGASVKVSCKASGYTFTDYYMHWVRQAPGQCLEWM |
| NO: 82 | Humanized VH 2 | GWINTNNGDTNYAQKFQGRVTMTRDTSISTAYMELSRLRSDDTAVYYCA |
| | | RALYTGSYWFAYWGQGTLVTVSS |
| SEQ ID | VB 13.2 | QVQLVQSGAEVKKPGASVKVSCKASGYTFTDYYMHWVRQAPGQCLEWM |
| NO: 83 | Humanized VH 3 | GIINTNNGDTSYAQKFQGRVTMTRDTSTSTVYMELSSLRSEDTAVYYCAR |
| | | ALYTGSYWFAYWGQGTLVTVSS |
| SEQ ID | VB 13.2 | QVQLVQSGAEVKKPGASVKVSCKASGYTFTDYYIHWLRQAPGQCLEWIGI |
| NO: 84 | Humanized VH 4 | VNTNNGDTNYNQKFQGRATLTVDKSSSTAYMELSSLRSEDTAVYYCARAL |
| | | YTGSYWFAYWGQGTLVTVSS |
| SEQ ID | VB 13.2 | QVQLVQSGAEVKKPGASVKVSCKASGYTFTDYYIHWVRQAPGQCLEWM |
| NO: 85 | Humanized VH 5 | GIVNTNNGDTNYNQKFQGRVTMTVDKSTSTAYMELSSLRSEDTAVYYCA |
| | | RALYTGSYWFAYWGQGTLVTVSS |
| SEQ ID | VB 13.2 | QVQLQQSGPGLVKPSETLSLTCTASGYTFTDYYIHWLRQPPGKCLEWIGIV |
| NO: 86 | Humanized VH 6 | NTNNGDTNYNQRLKSRATLSVDKSSSTASLKLSSVTAADTAVYYCARALY |
| | | TGSYWFAYWGQGTLVTVSS |
| SEQ ID | VB 13.2 | QVQLQESGPGLVKPSETLSLTCTASGYTFTDYYIHWIRQPPGKCLEWIGIVN |
| NO: 87 | Humanized VH 7 | TNNGDTNYNQRLKSRVTISVDKSKNQASLKLSSVTAADTAVYYCARALYT |
| | | GSYWFAYWGQGTLVTVSS |
| SEQ ID | VB 14 | QVQLVQSGAEVKKPGASVKLSCKASGFKTEDTYMHWVRQAPGQCLEWIG |
| NO: 88 | Humanized VH 1 | RIDPANGNTKYAPKFQDRVTITADTSANTAYLELSSLRSEDTAVYYCARGS |
| | | YYYAMDYWGQGTTVTVSS |
| SEQ ID | VB 14 | QVQLVQSGAEVKKPGASVKVSCKASGFKTEDTAMHWVRQAPGQCLEWM |
| NO: 89 | Humanized VH 2 | GWIDPANGNTKYSQKFQGRVTITRDTSASTAYMELSSLRSEDTAVYYCAR |
| | | GSYYYAMDYWGQGTTVTVSS |
| SEQ ID | VB 14 | QVQLVQSGAEVKKPGASVKVSCKASGFKTEDTYMHWVRQAPGQCLEWM |
| NO: 90 | Humanized VH 3 | GRIDPANGNTNYAQKFQGRVTMTRDTSISTAYMELSRLRSDDTAVYYCAR |
| | | GSYYYAMDYWGQGTTVTVSS |
| SEQ ID | VB 14 | QVQLVQSGAEVKKPGASVKVSCKASGFKTEDTYMHWVRQAPGQCLEWM |
| NO: 91 | Humanized VH 4 | GIIDPANGNTSYAQKFQGRVTMTRDTSTSTVYMELSSLRSEDTAVYYCARG |
| | | SYYYAMDYWGQGTTVTVSS |
| SEQ ID | VB 22 | EVQLVESGGGLVQPGGSLRLSCAASGFDFSRYWMNWVRQAPGKCLEWVS |
| NO: 92 | Humanized VH 1 | YINSDSNTINYADSVKGRFTISRDNAKNSLYLQMNSLRAEDTALYYCARGG |
| | | LLRDVWGQGTTVTVSS |
| SEQ ID | VB 22 | EVQLVESGGGLVQPGGSLRLSCAASGFDFSRYSMNWVRQAPGKCLEWVS |
| NO: 93 | Humanized VH 2 | YINSDSNTIYYADSVKGRFTISRDNAKNSLYLQMNSLRAEDTAVYYCARG |
| | | GLLRDVWGQGTMVTVSS |
| SEQ ID | VB 22 | EVQLVESGGGLVKPGGSLRLSCAASGFDFSRYSMNWVRQAPGKCLEWVSS |
| NO: 94 | Humanized VH 3 | INSDSNTIYYADSVKGRFTISRDNAKNSLYLQMNSLRAEDTAVYYCARGGL |
| | | LRDVWGQGTMVTVSS |
| SEQ ID | VB 11 | QVQLVQSGPEVKRPGASVKISCKASGYRFTDSAMHWVRQAPGQCLEWMG |
| NO: 95 | Humanized VH 1 | WISSYNGNTNYAQKFQGRVTMTVDTSTSTAYMELSRLTSEDTAIYYCARS |
| | | RDAMDYWGQGTLVTVSS |
| SEQ ID | VB 11 | QVQLVQSGAEVKKPGASVKVSCKASGYRFTDSGISWVRQAPGQCLEWMG |
| NO: 96 | Humanized VH 2 | WISSYNGNTNYAQKLQGRVTMTTDTSTSTAYMELRSLRSDDTAVYYCARS |
| | | RDAMDYWGQGTLVTVSS |
| SEQ ID | VB 11 | QVQLVQSGAEVKKPGASVKVSCKASGYRFTDSYMHWVRQAPGQCLEWM |
| NO: 97 | Humanized VH 3 | GWISSYNGNTNYAQKFQGRVTMTRDTSISTAYMELSRLRSDDTAVYYCAR |
| | | SRDAMDYWGQGTLVTVSS |
| SEQ ID | VB 1 Humanized | QVQLVQSGSELREPGASVKISCKASGYTFTDYYVHWVRQAPGKCLEWMG |
| NO: 98 | VH 1 | WINTYTGTPTYAQDFTGRFVFSLDTSASTAYLQISNLKAEDTAVYFCARSW |
| | | RRGIRGIGFDWGQGTLVTVSS |
| SEQ ID | VB 1 Humanized | QVQLVQSGSELKKPGASVKVSCKASGYTFTDYAMNWVRQAPGQCLEWM |
| NO: 99 | VH 2 | GWINTYTGTPTYAQGFTGRFVFSLDTSVSTAYLQISSLKAEDTAVYYCARS |
| | | WRRGIRGIGFDWGQGTLVTVSS |
| SEQ ID | VB 12 | EVQLVESGGGLVKPGGSLRLSCAASGFTFRSYGMSWVRQAPGKCLEWVA |
| NO: 100 | Humanized VH 1 | SISSGGSYTYYADSVKGRFTISRDNAKNSLYLQMSSLRAEDTAIYYCARHG |
| | | GNFFDYWGQGTLVTVSS |
| SEQ ID | VB 12 | EVQLVESGGGLVKPGGSLRLSCAASGFTFRSYSMNWVRQAPGKCLEWVSS |
| NO: 101 | Humanized VH 2 | ISSGGSYIYYADSVKGRFTISRDNAKNSLYLQMNSLRAEDTAVYYCARHGG |
| | | NFFDYWGQGTLVTVSS |
| SEQ ID | VB 12 | EVQLVESGGGLVQPGGSLRLSCAASGFTFRSYEMNWVRQAPGKCLEWVS |
| NO: 102 | Humanized VH 3 | YISSGGSYIYYADSVKGRFTISRDNAKNSLYLQMNSLRAEDTAVYYCARHG |
| | | GNFFDYWGQGTLVTVSS |
| SEQ ID | VB 12 | QVQLVESGGGLVKPGGSLRLSCAASGFTFRSYYMSWIRQAPGKCLEWVSY |
| NO: 103 | Humanized VH 4 | ISSGGSYIYYADSVKGRFTISRDNAKNSLYLQMNSLRAEDTAVYYCARHGG |
| | | NFFDYWGQGTLVTVSS |
| SEQ ID | VB 8 Humanized | EVQLVESGGGLVQPGGSLRLSCAASGFTFSNFGMHWVRQAPGKCLEWVA |
| NO: 104 | VH 1 | YISSGSSTIYYADSVKGRFTISRDNAKNTLFLQMNSLRAEDTAVYYCARRG |
| | | EGAMDYWGQGTLVTVSS |
| SEQ ID | VB 8 Humanized | EVQLVESGGGLVQPGGSLRLSCAASGFTFSNFSMNWVRQAPGKCLEWVSY |
| NO: 105 | VH 2 | ISSGSSTIYYADSVKGRFTISRDNAKNSLYLQMNSLRAEDTAVYYCARRGE |
| | | GAMDYWGQGTLVTVSS |
| SEQ ID | VB 8 Humanized | EVQLVESGGGLVKPGGSLRLSCAASGFTFSNFSMNWVRQAPGKCLEWVSY |
| NO: 106 | VH 3 | ISSGSSTIYYADSVKGRFTISRDNAKNSLYLQMNSLRAEDTAVYYCARRGE |
| | | GAMDYWGQGTLVTVSS |
| SEQ ID | VB 13.1 | EVQLVESGGGLVKPGGSLRLSCAASGFTFRSYAMSWVRQAPGKCLEWVA |
| NO: 107 | Humanized VH 1 | SISNGGDYIYYADSVKGRFTISRDNARNTLYLQMSSLRAEDTAVYYCARPS |
| | | YYSDPWFFDVWGRGTTVTVSS |
| SEQ ID | VB 13.1 | EVQLVESGGGLVKPGGSLRLSCAASGFTFRSYSMNWVRQAPGKCLEWVSS |
| NO: 108 | Humanized VH 2 | ISNGGDYIYYADSVKGRFTISRDNAKNSLYLQMNSLRAEDTAVYYCARPSY |
| | | YSDPWFFDVWGRGTLVTVSS |
| SEQ ID | VB 13.1 | EVQLVESGGGLVQPGGSLRLSCAASGFTFRSYAMSWVRQAPGKCLEWVS |
| NO: 109 | Humanized VH 3 | AISNGGDYTYYADSVKGRFTISRDNSKNTLYLQMNSLRAEDTAVYYCAKP |
| | | SYYSDPWFFDVWGRGTLVTVSS |
| SEQ ID | VB 13.1 | EVQLVESGGGLVQPGGSLRLSCAASGFTFRSYEMNWVRQAPGKCLEWVS |
| NO: 110 | Humanized VH 4 | YISNGGDYIYYADSVKGRFTISRDNAKNSLYLQMNSLRAEDTAVYYCARPS |
| | | YYSDPWFFDVWGRGTLVTVSS |
| SEQ ID | VB 5.1 | QVQLVQSGPELKKPGASVRVSCKASGYTFTDYYIHWVRQAPGRCLEWVG |
| NO: 111 | Humanized VH 1 | WINPYNGRTGYAQKFKGRVTLTRDKSSSTAYMDLRSLTSEDTAVYYCAR |
| | | WDGSSYFDYWGQGTLVTVSS |
| SEQ ID | VB 5.1 | QVQLVQSGAEVKKPGASVKVSCKASGYTFTDYYMHWVRQAPGQCLEWM |
| NO: 112 | Humanized VH 2 | GWINPYNGRTNYAQKFQGRVTMTRDTSISTAYMELSRLRSDDTAVYYCAR |
| | | WDGSSYFDYWGQGTLVTVSS |
| SEQ ID | VB 5.1 | QVQLVQSGAEVKKPGASVKVSCKASGYTFTDYYMHWVRQAPGQCLEWM |
| NO: 113 | Humanized VH 3 | GIINPYNGRTSYAQKFQGRVTMTRDTSTSTVYMELSSLRSEDTAVYYCAR |
| | | WDGSSYFDYWGQGTLVTVSS |
| SEQ ID | VB 5.1 | QVQLVQSGAEVKKPGASVKVSCKASGYTFTDYGISWVRQAPGQCLEWMG |
| NO: 114 | Humanized VH 4 | WINPYNGRTNYAQKLQGRVTMTTDTSTSTAYMELRSLRSDDTAVYYCAR |
| | | WDGSSYFDYWGQGTLVTVSS |
| SEQ ID | VB 5.1 | QVQLVQSGAEVKKPGASVKVSCKASGYTFTDYYMHWVRQAPGQCLEWM |
| NO: 115 | Humanized VH 5 | GWINPYNGRTNYAQKFQGRVTMTRDTSISTAYMELSRLRSDDTAVYYCAR |
| | | WDGSSYFDYWGQGTLVTVSS |
| SEQ ID | VB 9 Humanized | QVQLVQSGAELKRPGASVKLSCTASGFNIKNTFMHWVRQAPGQCLEWIGR |
| NO: 116 | VH 1 | IDPTNGYTKFAPKFQGRVTLTADTSSNTVYLQLSSLTSEDTAIYYCARDYD |
| | | APWFAYWGQGTLVIVSS |
| SEQ ID | VB 9 Humanized | QVQLVQSGAEVKKPGASVKVSCKASGFNIKNTYMHWVRQAPGQCLEWM |
| NO: 117 | VH 2 | GIIDPTNGYTSYAQKFQGRVTMTRDTSTSTVYMELSSLRSEDTAVYYCARD |
| | | YDAPWFAYWGQGTLVTVSS |
| SEQ ID | VB 9 Humanized | QVQLVQSGAEVKKPGASVKVSCKASGFNIKNTYMHWVRQAPGQCLEWM |
| NO: 118 | VH 3 | GRIDPTNGYTNYAQKFQGRVTMTRDTSISTAYMELSRLRSDDTAVYYCAR |
| | | DYDAPWFAYWGQGTLVTVSS |
| SEQ ID | VB 9 Humanized | QVQLVQSGAEVKKPGASVKVSCKASGFNIKNTAMHWVRQAPGQCLEWM |
| NO: 119 | VH 4 | GWIDPTNGYTKYSQKFQGRVTITRDTSASTAYMELSSLRSEDTAVYYCAR |
| | | DYDAPWFAYWGQGTLVTVSS |
| SEQ ID | VB 17 | QVQLQESGPGLVKPSQTLSLTCSVSGYSITSGYFWNWIRQPPGKCLEWIGYI |
| NO: 120 | Humanized VH 1 | SYDGSTNYNPSLKSRISISVDTSKNQFSLKLNSVTAADTAVYYCASPSPGTG |
| | | YAVDYWGQGTLVTVSS |
| SEQ ID | VB 17 | QVQLQESGPGLVKPSETLSLTCTVSGYSITSGYWSWIRQPPGKCLEWIGYIS |
| NO: 121 | Humanized VH 2 | YDGSTNYNPSLKSRVTISVDTSKNQFSLKLSSVTAADTAVYYCARPSPGTG |
| | | YAVDYWGQGTLVTVSS |
| SEQ ID | VB 17 | QVQLQESGPGLVKPSQTLSLTCAVSGYSITSGYYWGWIRQPPGKCLEWIGY |
| NO: 122 | Humanized VH 3 | ISYDGSTYYNPSLKSRVTISVDTSKNQFSLKLSSVTAADTAVYYCARPSPGT |
| | | GYAVDYWGQGTLVTVSS |
| SEQ ID | VB 3 Humanized | QVQLVQSGPEVKKPGASVKISCKASGYTFSGSWMHWVRQAPGKCLEWIG |
| NO: 123 | VH 1 | RIYPGDGDTDYSQKFQGRVTLTADKSSSTAYMELSSLTSEDTAVYFCARSG |
| | | YFNYVPVFDYWGQGTLVTVSS |
| SEQ ID | VB 3 Humanized | QVQLVQSGAEVKKPGASVKVSCKASGYTFSGSYMHWVRQAPGQCLEWM |
| NO: 124 | VH 2 | GRIYPGDGDTNYAQKFQGRVTMTRDTSISTAYMELSRLRSDDTAVYYCAR |
| | | SGYFNYVPVFDYWGQGTLVTVSS |
| SEQ ID | VB 3 Humanized | QVQLVQSGAEVKKPGASVKVSCKASGYTFSGSAMHWVRQAPGQCLEWM |
| NO: 125 | VH 3 | GWIYPGDGDTKYSQKFQGRVTITRDTSASTAYMELSSLRSEDTAVYYCARS |
| | | GYFNYVPVFDYWGQGTLVTVSS |
| SEQ ID | VB 7.1 | EVQLVESGGGLVKPGGSLRLSCAASGFTFSDYYMNWVRQTPGKCLEWVA |
| NO: 126 | Humanized VH 1 | SISGGGSYTYYADSVKGRFTISRDNAKNSLYLQMSSLRAEDTAVYFCARER |
| | | DIYYGNFNAMVYWGQGTLVTVSS |
| SEQ ID | VB 7.1 | EVQLVESGGGLVKPGGSLRLSCAASGFTFSDYYMNWVRQAPGKCLEWVS |
| NO: 127 | Humanized VH 2 | SISGGGSYIYYADSVKGRFTISRDNAKNSLYLQMNSLRAEDTAVYYCARER |
| | | DIYYGNFNAMVYWGRGTLVTVSS |
| SEQ ID | VB 7.1 | EVQLVESGGGLVKPGGSLRLSCAASGFTFSDYSMNWVRQAPGKCLEWVSS |
| NO: 128 | Humanized VH 3 | ISGGGSYIYYADSVKGRFTISRDNAKNSLYLQMNSLRAEDTAVYYCARERD |
| | | IYYGNFNAMVYWGRGTLVTVSS |
| SEQ ID | VB 7.1 | QVQLVESGGGLVKPGGSLRLSCAASGFTFSDYYMSWIRQAPGKCLEWVSY |
| NO: 129 | Humanized VH 4 | ISGGGSYIYYADSVKGRFTISRDNAKNSLYLQMNSLRAEDTAVYYCARERD |
| | | IYYGNFNAMVYWGQGTLVTVSS |
| SEQ ID | VB 5.3 | QVQLVQSGPELKKPGASVKISCKASGYAFSSSWMHWVRQAPGQCLEWIGR |
| NO: 130 | Humanized VH 1 | IYPGDGDTKYAQKFQGRVTLTADKSSSTAYMELSSLTSEDTAVYFCARRGT |
| | | GGWYFDVWGKGTLVTVSS |
| SEQ ID | VB 5.3 | QVQLVQSGAEVKKPGASVKVSCKASGYAFSSSAMHWVRQAPGQCLEWM |
| NO: 131 | Humanized VH 2 | GWIYPGDGDTKYSQKFQGRVTITRDTSASTAYMELSSLRSEDTAVYYCAR |
| | | RGTGGWYFDVWGRGTLVTVSS |
| SEQ ID | hCAR-Vb2-(V1- | DIVLTQSPASLAVSLGERATISCRASKSVSILGTHLIHWYQQKPGQPPKLLIY |
| NO: 132 | 46*01_1_V7- | AASNLESGVPDRFSGSGSGTDFTLTISRVEAEDVAVYYCQQSIEDPWTFGC |
| | 3*01_1) VL | GTKLEIK |
| SEQ ID | hCAR-Vb2-(V1- | DIVLTQSPASLAVSPGERATISCRASKSVSILGTHLIHWYQQKPGQPPKLLIY |
| NO: 133 | 46*01_1_V7- | AASNLESGVPDRFSGSGSGTDFTLTISRVEAEDVAVYYCQQSIEDPWTFGC |
| | 3*01_2) VL | GTKVEIK |
| SEQ ID | hCAR-Vb2-(V1- | DIVMTQSPDSLAVSLGERATINCRASKSVSILGTHLIHWYQQKPGQPPKLLI |
| NO: 134 | 46*01_1_V4- | YAASNLESGVPDRFSGSGSGTDFTLTISSLQAEDVAVYYCQQSIEDPWTFGC |
| | 1*01_1) VL | GTKVEIK |
| SEQ ID | hCAR-Vb2-(V1- | EIVLTQSPATLSLSPGERATLSCRASKSVSILGTHLIHWYQQKPGQSPRLLIY |
| NO: 135 | 46*01_1_V3D- | AASNLESGVPDRFSGSGSGTDFTLTISRLEAEDVAVYYCQQSIEDPWTFGCG |
| | 20*02) VL | TKVEIK |
| SEQ ID | hCAR-Vb2-(V1- | DIVLTQSPASLAVSLGERATISCRASKSVSILGTHLIHWYQQKPGQPPKLLIY |
| NO: 136 | 2*06_V7-3*01_1) | AASNLESGVPDRFSGSGSGTDFTLTISRVEAEDVAVYYCQQSIEDPWTFGC |
| | VL | GTKLEIK |
| SEQ ID | hCAR-Vb2-(V1- | DIVLTQSPASLAVSPGERATISCRASKSVSILGTHLIHWYQQKPGQPPKLLIY |
| NO: 137 | 2*06_V7-3*01_2) | AASNLESGVPDRFSGSGSGTDFTLTISRVEAEDVAVYYCQQSIEDPWTFGC |
| | VL | GTKVEIK |
| SEQ ID | hCAR-Vb2-(V1- | DIVMTQSPDSLAVSLGERATINCRASKSVSILGTHLIHWYQQKPGQPPKLLI |
| NO: 138 | 2*06_V4-1*01_1) | YAASNLESGVPDRFSGSGSGTDFTLTISSLQAEDVAVYYCQQSIEDPWTFGC |
| | VL | GTKVEIK |
| SEQ ID | hCAR-Vb2-(V1- | EIVLTQSPATLSLSPGERATLSCRASKSVSILGTHLIHWYQQKPGQSPRLLIY |
| NO: 139 | 2*06_V3D- | AASNLESGVPDRFSGSGSGTDFTLTISRLEAEDVAVYYCQQSIEDPWTFGCG |
| | 20*02) VL | TKVEIK |
| SEQ ID | hCAR-Vb2-(V1- | DIVLTQSPASLAVSLGERATISCRASKSVSILGTHLIHWYQQKPGQPPKLLIY |
| NO: 140 | 3*01_V7-3*01_1) | AASNLESGVPDRFSGSGSGTDFTLTISRVEAEDVAVYYCQQSIEDPWTFGC |
| | VL | GTKLEIK |
| SEQ ID | hCAR-Vb2-(V1- | DIVLTQSPASLAVSPGERATISCRASKSVSILGTHLIHWYQQKPGQPPKLLIY |
| NO: 141 | 3*01_V7-3*01_2) | AASNLESGVPDRFSGSGSGTDFTLTISRVEAEDVAVYYCQQSIEDPWTFGC |
| | VL | GTKVEIK |
| SEQ ID | hCAR-Vb2-(V1- | DIVMTQSPDSLAVSLGERATINCRASKSVSILGTHLIHWYQQKPGQPPKLLI |
| NO: 142 | 3*01_V4-1*01_1) | YAASNLESGVPDRFSGSGSGTDFTLTISSLQAEDVAVYYCQQSIEDPWTFGC |
| | VL | GTKVEIK |
| SEQ ID | hCAR-Vb2-(V1- | EIVLTQSPATLSLSPGERATLSCRASKSVSILGTHLIHWYQQKPGQSPRLLIY |
| NO: 143 | 3*01_V3D- | AASNLESGVPDRFSGSGSGTDFTLTISRLEAEDVAVYYCQQSIEDPWTFGCG |
| | 20*02) VL | TKVEIK |
| SEQ ID | hCAR-Vb2-(V1- | DIVLTQSPDSLAVSLGERATINCKASKSVSILGTHLIHWYQQKPGQPPKLLIY |
| NO: 144 | 46*01_2_V4- | AASNLESGVPDRFSGSGSETVFTLTISSLQAEDVATYFCQQSIEDPWTFGCG |
| | 1*01_2) VL | TKLEIK |
| SEQ ID | hCAR-Vb2-(V1- | DIVLTQSPDSLAVSLGERATINCKASKSVSILGTHLIHWYQQKPGQPPKLLIY |
| NO: 145 | 46*01_2_V4- | AASNRESGVPDRFSGSGSETDFTLTISSLQAEDVAVYYCQQSIEDPWTFGCG |
| | 1*01_3) VL | TKLEIK |
| SEQ ID | hCAR-Vb2-(V1- | DIQLTQSPSSLSASVGDRVTITCRASKSVSILGTHLIHWYQQKPGKAPKLLIY |
| NO: 146 | 46*01_2_V1- | AASNLESGVPSRFSGSGSETVFTLTISSLQPEDFATYFCQQSIEDPWTFGCGT |
| | 39*01_1) VL | KLEIK |
| SEQ ID | hCAR-Vb2-(V1- | DIQLTQSPSSLSASVGDRVTITCRASKSVSILGTHLIHWYQQKPGKAPKLLIY |
| NO: 147 | 46*01_2_V1- | AASNLESGVPSRFSGSGSETDFTLTISSLQPEDFATYYCQQSIEDPWTFGCGT |
| | 39*01_2) VL | KLEIK |
| SEQ ID | hCAR-Vb2-(V1- | DIVLTQSPDSLAVSLGERATINCKASKSVSILGTHLIHWYQQKPGQPPKLLIY |
| NO: 148 | 46*01_3_V4- | AASNLESGVPDRFSGSGSETVFTLTISSLQAEDVATYFCQQSIEDPWTFGCG |
| | 1*01_2) VL | TKLEIK |
| SEQ ID | hCAR-Vb2-(V1- | DIVLTQSPDSLAVSLGERATINCKASKSVSILGTHLIHWYQQKPGQPPKLLIY |
| NO: 149 | 46*01_3_V4- | AASNRESGVPDRFSGSGSETDFTLTISSLQAEDVAVYYCQQSIEDPWTFGCG |
| | 1*01_3) VL | TKLEIK |
| SEQ ID | hCAR-Vb2-(V1- | DIQLTQSPSSLSASVGDRVTITCRASKSVSILGTHLIHWYQQKPGKAPKLLIY |
| NO: 150 | 46*01_3_V1- | AASNLESGVPSRFSGSGSETVFTLTISSLQPEDFATYFCQQSIEDPWTFGCGT |
| | 39*01_1) VL | KLEIK |
| SEQ ID | hCAR-Vb2-(V1- | DIQLTQSPSSLSASVGDRVTITCRASKSVSILGTHLIHWYQQKPGKAPKLLIY |
| NO: 151 | 46*01_3_V1- | AASNLESGVPSRFSGSGSETDFTLTISSLQPEDFATYYCQQSIEDPWTFGCGT |
| | 39*01_2) VL | KLEIK |
| SEQ ID | hCAR-Vb2-(V7- | DIVLTQSPDSLAVSLGERATINCKASKSVSILGTHLIHWYQQKPGQPPKLLIY |
| NO: 152 | 4-1*02_1_V4- | AASNLESGVPDRFSGSGSETVFTLTISSLQAEDVATYFCQQSIEDPWTFGCG |
| | 1*01_2) VL | TKLEIK |
| SEQ ID | hCAR-Vb2-(V7- | DIVLTQSPDSLAVSLGERATINCKASKSVSILGTHLIHWYQQKPGQPPKLLIY |
| NO: 153 | 4-1*02_1_V4- | AASNRESGVPDRFSGSGSETDFTLTISSLQAEDVAVYYCQQSIEDPWTFGCG |
| | 1*01_3) VL | TKLEIK |
| SEQ ID | hCAR-Vb2-(V7- | DIQLTQSPSSLSASVGDRVTITCRASKSVSILGTHLIHWYQQKPGKAPKLLIY |
| NO: 154 | 4-1*02_1_V1- | AASNLESGVPSRFSGSGSETVFTLTISSLQPEDFATYFCQQSIEDPWTFGCGT |
| | 39*01_1) VL | KLEIK |
| SEQ ID | hCAR-Vb2-(V7- | DIQLTQSPSSLSASVGDRVTITCRASKSVSILGTHLIHWYQQKPGKAPKLLIY |
| NO: 155 | 4-1*02_1_V1- | AASNLESGVPSRFSGSGSETDFTLTISSLQPEDFATYYCQQSIEDPWTFGCGT |
| | 39*01_2) VL | KLEIK |
| SEQ ID | hCAR-Vb2-(V7- | DIVLTQSPDSLAVSLGERATINCKASKSVSILGTHLIHWYQQKPGQPPKLLIY |
| NO: 156 | 4-1*02_2_V4- | AASNLESGVPDRFSGSGSETVFTLTISSLQAEDVATYFCQQSIEDPWTFGCG |
| | 1*01_2) VL | TKLEIK |
| SEQ ID | hCAR-Vb2-(V7- | DIVLTQSPDSLAVSLGERATINCKASKSVSILGTHLIHWYQQKPGQPPKLLIY |
| NO: 157 | 4-1*02_2_V4- | AASNRESGVPDRFSGSGSETDFTLTISSLQAEDVAVYYCQQSIEDPWTFGCG |
| | 1*01_3) VL | TKLEIK |
| SEQ ID | hCAR-Vb2-(V7- | DIQLTQSPSSLSASVGDRVTITCRASKSVSILGTHLIHWYQQKPGKAPKLLIY |
| NO: 158 | 4-1*02_2_V1- | AASNLESGVPSRFSGSGSETVFTLTISSLQPEDFATYFCQQSIEDPWTFGCGT |
| | 39*01_1) VL | KLEIK |
| SEQ ID | hCAR-Vb2-(V7- | DIQLTQSPSSLSASVGDRVTITCRASKSVSILGTHLIHWYQQKPGKAPKLLIY |
| NO: 159 | 4-1*02_2_V1- | AASNLESGVPSRFSGSGSETDFTLTISSLQPEDFATYYCQQSIEDPWTFGCGT |
| | 39*01_2) VL | KLEIK |
| SEQ ID | VB 7.2 | DIQMTQSPSFLSASVGDRVTITCKASQDVYTAVAWYQQKPGQAPKLLIYSA |
| NO: 160 | Humanized VL 1 | SNRYTGVPSRFSGSGSGTDFTLTISSLQAEDLATYYCQQHYTTPRTFGCGTK |
| | | VEIK |
| SEQ ID | VB 7.2 | DIQMTQSPSSLSASVGDRVTITCKASQDVYTAVAWYQQKPGKAPKLLIYSA |
| NO: 161 | Humanized VL 2 | SNRYTGVPSRFSGSGSGTDFTFTISSLQPEDIATYYCQQHYTTPRTFGCGTKL |
| | | EIK |
| SEQ ID | VB 7.2 | DIQMTQSPSSVSASVGDRVTITCKASQDVYTAVAWYQQKPGKAPKLLIYS |
| NO: 162 | Humanized VL 3 | ASNRYTGVPSRFSGSGSGTDFTLTISSLQPEDFATYYCQQHYTTPRTFGCGT |
| | | KLEIK |
| SEQ ID | VB 7.2 | AIQLTQSPSSLSASVGDRVTITCKASQDVYTAVAWYQQKPGKAPKLLIYSA |
| NO: 163 | Humanized VL 4 | SNRYTGVPSRFSGSGSGTDFTLTISSLQPEDFATYYCQQHYTTPRTFGCGTK |
| | | LEIK |
| SEQ ID | VB 4 murine VL | DIQMTQSPHSLSASLGETVSIECLASEGISNFLAWYQQKPGKSPQLLIYYTSS |
| NO: 164 | | LQDGVPSRFSGSGSGTQYSLKISNMQPEDEGVYYCQQGYKFPRTFGCGTKL |
| | | ELK |
| SEQ ID | VB 4 murine VL1 | DIQMTQSPSSLSASVGDTVTITCLASEGISNFLAWYQQKPGKAPKLLIYYTS |
| NO: 165 | | SLQDGVPSRFSGSGSGTDFSLTISNLQPEDFGTYYCQQGYKFPRTFGCGTKV |
| | | EIK |
| SEQ ID | VB 4 Humanized | DIQMTQSPSSVSASVGDRVTITCLASEGISNFLAWYQQKPGKAPKLLIYYTS |
| NO: 166 | VL 1 | SLQDGVPSRFSGSGSGTDFTLTISSLQPEDFATYYCQQGYKFPRTFGCGTKL |
| | | EIK |
| SEQ ID | VB 4 Humanized | DIQMTQSPSSLSASVGDRVTITCLASEGISNFLAWFQQKPGKAPKSLIYYTSS |
| NO: 167 | VL 2 | LQDGVPSRFSGSGSGTDFTLTISSLQPEDFATYYCQQGYKFPRTFGCGTKLE |
| | | IK |
| SEQ ID | VB 13.2 | EIVLTQSPATLSVSPGEKVTITCSASSSISSTNLHWYQQKPGQSPRLLIYGTS |
| NO: 168 | Humanized VL 1 | NLASGVPDRFSGSGSGTDFTLTISSLEAEDFATYYCQQWSRYPLTFGCGTKL |
| | | EIK |
| SEQ ID | VB 13.2 | EIVLTQSPATLSLSPGERATLSCSASSSISSTNLHWYQQKPGQAPRLLIYGTS |
| NO: 169 | Humanized VL 2 | NLASGIPDRFSGSGSGTDFTLTISSLEPEDFAVYYCQQWSRYPLTFGCGTKL |
| | | EIK |
| SEQ ID | VB 13.2 | EIVLTQSPATLSLSPGERATLSCSASSSISSTNLHWYQQKPGQAPRLLIYGTS |
| NO: 170 | Humanized VL 3 | NLASGIPDRFSGSGSGTDFTLTISRLEPEDFAVYYCQQWSRYPLTFGCGTKL |
| | | EIK |
| SEQ ID | VB 13.2 | EIVLTQSPGTLSLSPGERATLSCSASSSISSTNLHWYQQKPGQAPRLLIYGTS |
| NO: 171 | Humanized VL 4 | NLASGIPDRFSGSGSGTDFTLTISRLEPEDFAVYYCQQWSRYPLTFGCGTKL |
| | | EIK |
| SEQ ID | VB 13.2 | EIQLTQSPSSLSASVGDRVTITCRASSSISSTNLHWYQQKPGKAPKPWIYGTS |
| NO: 172 | Humanized VL 5 | NLASGVPSRFSGSGSGTSYSLTISSLQPEDFATYYCQQWSRYPLTFGCGTKL |
| | | EIK |
| SEQ ID | VB 13.2 | AIQLTQSPSSLSASVGDRVTITCRASSSISSTNLHWYQQKPGKAPKPLIYGTS |
| NO: 173 | Humanized VL 6 | NLASGVPSRFSGSGSGTDYTLTISSLQPEDFATYYCQQWSRYPLTFGCGTKL |
| | | EIK |
| SEQ ID | VB 13.2 | EIVLTQSPATLSLSPGERATLSCRASSSISSTNLHWYQQKPGQAPRPWIYGTS |
| NO: 174 | Humanized VL 7 | NLASGVPARFSGSGSGTSYSLTISSLEPEDFATYYCQQWSRYPLTFGCGTKL |
| | | EIK |
| SEQ ID | VB 13.2 | EIVLTQSPATLSLSPGERATLSCRASSSISSTNLHWYQQKPGQAPRPLIYGTS |
| NO: 175 | Humanized VL 8 | NRATGIPARFSGSGSGTDYTLTISSLEPEDFAVYYCQQWSRYPLTFGCGTKL |
| | | EIK |
| SEQ ID | VB 14 | DIVLTQSPASLAVSPGERATISCRASESVDSYGNSFMHWYQQKPGQPPKLLI |
| NO: 176 | Humanized VL 1 | YRASNLESGVPDRFSGSGSGTDFTLTISRVEADDVAVYYCQQSNEDPYTFG |
| | | CGTKLEIK |
| SEQ ID | VB 14 | DIVLTQSPASLAVSPGERATISCRASESVDSYGNSFMHWYQQKPGQPPKLLI |
| NO: 177 | Humanized VL 2 | YRASNLESGVPDRFSGSGSGTDFTLTISRVEAEDVAVYYCQQSNEDPYTFG |
| | | CGTKLEIK |
| SEQ ID | VB 14 | DIVMTQSPDSLAVSLGERATINCRASESVDSYGNSFMHWYQQKPGQPPKLL |
| NO: 178 | Humanized VL 3 | IYRASNLESGVPDRFSGSGSGTDFTLTISSLQAEDVAVYYCQQSNEDPYTFG |
| | | CGTKLEIK |
| SEQ ID | VB 14 | EIVLTQSPATLSLSPGERATLSCRASESVDSYGNSFMHWYQQKPGQPPRLLI |
| NO: 179 | Humanized VL 4 | YRASNLESGVPDRFSGSGSGTDFTLTISRLEAEDVAVYYCQQSNEDPYTFG |
| | | CGTKLEIK |
| SEQ ID | VB 14 | EIVLTQSPATLSLSPGERATLSCRASESVDSYGNSFMHWYQQKPGQPPRLLI |
| NO: 180 | Humanized VL 5 | YRASNLESGVPDRFSGSGSGTDFTLTISSLEAEDFAVYYCQQSNEDPYTFGC |
| | | GTKLEIK |
| SEQ ID | VB 14 | EIVLTQSPGTLSLSPGERATLSCRASESVDSYGNSFMHWYQQKPGQPPRLLI |
| NO: 181 | Humanized VL 6 | YRASNLESGVPDRFSGSGSGTDFTLTISRLEAEDVAVYYCQQSNEDPYTFG |
| | | CGTKLEIK |
| SEQ ID | VB 22 | QAVVTQEPSLTVSPGGTVTLTCRSSTGAVTTSNYANWFQQKPGHAPRTLIY |
| NO: 182 | Humanized VL 1 | GTNNRAPGVPARFSGSLLGDKAALTITGAQTEDEADYFCALWYSNHWVFG |
| | | CGTKLTVL |
| SEQ ID | VB 22 | QAVVTQEPSLTVSPGGTVTLTCRSSTGAVTTSNYANWFQQKPGQAPRTLIY |
| NO: 183 | Humanized VL 2 | GTNNRAPWTPARFSGSLLGGKAALTLSGAQPEDEAEYYCALWYSNHWVF |
| | | GCGTKLTVL |
| SEQ ID | VB 22 | QTVVTQEPSLTVSPGGTVTLTCRSSTGAVTTSNYANWFQQKPGQAPRALIY |
| NO: 184 | Humanized VL 3 | GTNNRAPWTPARFSGSLLGGKAALTLSGVQPEDEAEYYCALWYSNHWVF |
| | | GCGTKLTVL |
| SEQ ID | VB 22 | QTVVTQEPSFSVSPGGTVTLTCRSSTGAVTTSNYANWYQQTPGQAPRTLIY |
| NO: 185 | Humanized VL 4 | GTNNRAPGVPDRFSGSILGNKAALTITGAQADDESDYYCALWYSNHWVFG |
| | | CGTKLTVL |
| SEQ ID | VB 11 | DIQMTQSPSSLSASVGDRVTITCKASQDINSYLSWFQQKPGKAPKLLIYRAN |
| NO: 186 | Humanized VL 1 | RLVDGVPSRFSGSGSGTDYTLTISSLQPEDFATYYCLQYDEFPFTFGCGTRL |
| | | EIK |
| SEQ ID | VB 11 | DIQMTQSPSSLSASVGDRVTITCKASQDINSYLSWFQQKPGKAPKSLIYRAN |
| NO: 187 | Humanized VL 2 | RLVDGVPSRFSGSGSGTDFTLTISSLQPEDFATYYCLQYDEFPFTFGCGTRLE |
| | | IK |
| SEQ ID | VB 11 | DIQMTQSPSSLSASVGDRVTITCKASQDINSYLSWYQQKPGKAPKLLIYRA |
| NO: 188 | Humanized VL 3 | NRLVDGVPSRFSGSGSGTDFTFTISSLQPEDIATYYCLQYDEFPFTFGCGTRL |
| | | EIK |
| SEQ ID | VB 11 | DIQMTQSPSSLSASVGDRVTITCKASQDINSYLSWYQQKPGKAPKLLLYRA |
| NO: 189 | Humanized VL 4 | NRLVDGVPSRFSGSGSGTDYTLTISSLQPEDFATYYCLQYDEFPFTFGCGTR |
| | | LEIK |
| SEQ ID | VB 1 Humanized | DVQMTQSPSTLSASVGESVTITCKASKSINKYLAWYQQKPGKAPKLLIYDG |
| NO: 190 | VL 1 | STLQSGVPSRFSGSGSGTDFTLTISGLQPEDFATYYCQQHNEYPPTFGCGTK |
| | | LEIK |
| SEQ ID | VB 1 Humanized | DIQMTQSPSSLSASVGDRVTITCKASKSINKYLAWYQQKPGKAPKLLIYDG |
| NO: 191 | VL 2 | STLQSGVPSRFSGSGSGTDFTLTISSLQPEDFATYYCQQHNEYPPTFGCGTK |
| | | LEIK |
| SEQ ID | VB 1 Humanized | DIQMTQSPSTLSASVGDRVTITCKASKSINKYLAWYQQKPGKAPKLLIYDG |
| NO: 192 | VL 3 | STLQSGVPSRFSGSGSGTEFTLTISSLQPDDFATYYCQQHNEYPPTFGCGTK |
| | | LEIK |
| SEQ ID | VB 1 Humanized | DIQLTQSPSFLSASVGDRVTITCKASKSINKYLAWYQQKPGKAPKLLIYDGS |
| NO: 193 | VL 4 | TLQSGVPSRFSGSGSGTEFTLTISSLQPEDFATYYCQQHNEYPPTFGCGTKLE |
| | | IK |
| SEQ ID | VB 1 Humanized | DIQMTQSPSSLSASVGDRVTITCKASKSINKYLAWFQQKPGKAPKSLIYDGS |
| NO: 194 | VL 5 | TLQSGVPSRFSGSGSGTDFTLTISSLQPEDFATYYCQQHNEYPPTFGCGTKL |
| | | EIK |
| SEQ ID | VB 12 | EIVLTQSPSFLSASPGEKVTITCSVSSSVSYMEIWYQQKPGQAPKLLIYDTSK |
| NO: 195 | Humanized VL 1 | LASGVPARFSGSGSGTDFTLTISSLEAEDFATYYCQQWSSNPQYTFGCGTKL |
| | | EIK |
| SEQ ID | VB 12 | AIQLTQSPSSLSASVGDRVTITCSVSSSVSYMEIWYQQKPGKAPKLLIYDTS |
| NO: 196 | Humanized VL 2 | KLASGVPSRFSGSGSGTDFTLTISSLQPEDFATYYCQQWSSNPQYTFGCGTK |
| | | LEIK |
| SEQ ID | VB 12 | EIVLTQSPATLSLSPGERATLSCSVSSSVSYMEIWYQQKPGQAPRLLIYDTSK |
| NO: 197 | Humanized VL 3 | LASGIPARFSGSGSGTDFTLTISSLEPEDFAVYYCQQWSSNPQYTFGCGTKL |
| | | EIK |
| SEQ ID | VB 12 | DIQMTQSPSSLSASVGDRVTITCSVSSSVSYMEIWYQQKPGKAPKLLIYDTS |
| NO: 198 | Humanized VL 4 | KLASGVPSRFSGSGSGTDFTLTISSLQPEDFATYYCQQWSSNPQYTFGCGTK |
| | | LEIK |
| SEQ ID | VB 12 | DIQLTQSPSFLSASVGDRVTITCSVSSSVSYMEIWYQQKPGKAPKLLIYDTS |
| NO: 199 | Humanized VL 5 | KLASGVPSRFSGSGSGTEFTLTISSLQPEDFATYYCQQWSSNPQYTFGCGTK |
| | | LEIK |
| SEQ ID | VB 8 Humanized | EIVLTQSPAILSVSLGEKVTLSCRASSSVNYIYWYQQKPGKAPKLLIYYTSN |
| NO: 200 | VL 1 | LAPGVPTRFSGSGSGTDFSLTISSLQPEDAATYYCQQFTSSPFTFGCGTKLEI |
| | | K |
| SEQ ID | VB 8 Humanized | EIVLTQSPATLSLSPGERATLSCRASSSVNYIYWYQQKPGQAPRLLIYYTSN |
| NO: 201 | VL 2 | LAPGIPARFSGSGSGTDFTLTISSLEPEDFAVYYCQQFTSSPFTFGCGTKLEIK |
| SEQ ID | VB 8 Humanized | DIQLTQSPSSLSASVGDRVTITCRASSSVNYIYWYQQKPGKAPKLLIYYTSN |
| NO: 202 | VL 3 | LAPGVPSRFSGSGSGTDFTLTISSLQPEDFATYYCQQFTSSPFTFGCGTKLEI |
| | | K |
| SEQ ID | VB 13.1 | EIVLTQSPAILSASPGEKVTLTCSAGSSVSFMEIWYQQKPGQSPKLLIYDTSK |
| NO: 203 | Humanized VL 1 | LASGVPGRFSGSGSGTDFSLTISRLEPDDAAYYYLQGSGFPLTFGCGTKLEI |
| | | K |
| SEQ ID | VB 13.1 | EIVLTQSPATLSLSPGERATLSCSAGSSVSFMEIWYQQKPGQAPRLLIYDTSK |
| NO: 204 | Humanized VL 2 | LASGIPARFSGSGSGTDFTLTISSLEPEDFAVYYCLQGSGFPLTFGCGTKLEI |
| | | K |
| SEQ ID | VB 13.1 | EIVLTQSPATLSLSPGERATLSCSAGSSVSFMEIWYQQKPGQAPRLLIYDTSK |
| NO: 205 | Humanized VL 3 | LASGIPDRFSGSGSGTDFTLTISRLEPEDFAVYYCLQGSGFPLTFGCGTKLEI |
| | | K |
| SEQ ID | VB 5.1 | EIVLTQSPAILAVSLGEKVTITCSASSSVSYMHWYQQKPGKAPKLLIYEISKL |
| NO: 206 | Humanized VL 1 | ASGVPARFSGSGSGTSFSLTISSLQAEDAATYYCQQWNYPLLTFGCGTKLEI |
| | | K |
| SEQ ID | VB 5.1 | DIQLTQSPSFLSASVGDRVTITCSASSSVSYMHWYQQKPGKAPKLLIYEISK |
| NO: 207 | Humanized VL 2 | LASGVPSRFSGSGSGTEFTLTISSLQPEDFATYYCQQWNYPLLTFGCGTKLEI |
| | | K |
| SEQ ID | VB 5.1 | DIQLTQSPSSLSASVGDRVTITCSASSSVSYMHWYQQKPGKAPKLLIYEISK |
| NO: 208 | Humanized VL 3 | LASGVPSRFSGSGSGTDFTLTISSLQPEDFATYYCQQWNYPLLTFGCGTKLE |
| | | IK |
| SEQ ID | VB 9 Humanized | ETVMTQSPASLSVAPGEKVTITCISSTDIDDDMNWYQQKPGEPPKLLISEGN |
| NO: 209 | VL 1 | TLRPGVPSRFSGSGYGTDFTFTINNLQSEDVADYYCLQSDNMPLTFGCGTK |
| | | LEIK |
| SEQ ID | VB 9 Humanized | ETTLTQSPAFMSATPGDKVNISCISSTDIDDDMNWYQQKPGEAPIFIIQEGNT |
| NO: 210 | VL 2 | LRPGIPPRFSGSGYGTDFTLTINNIESEDAAYYFCLQSDNMPLTFGCGTKLEI |
| | | K |
| SEQ ID | VB 9 Humanized | DIQMTQSPSSLSASVGDRVTITCISSTDIDDDMNWYQQKPGKAPKLLIYEGN |
| NO: 211 | VL 3 | TLRPGVPSRFSGSGSGTDFTFTISSLQPEDIATYYCLQSDNMPLTFGCGTKLE |
| | | IK |
| SEQ ID | VB 9 Humanized | DVVMTQSPAFLSVTPGEKVTITCISSTDIDDDMNWYQQKPGQAPKLLIKEG |
| NO: 212 | VL 4 | NTLRPGVPSRFSGSGSGTDFTLTISSLEAEDAATYYCLQSDNMPLTFGCGTK |
| | | LEIK |
| SEQ ID | VB 17 | DVVMTQTPLSLPVTLGQPASISCRSSQSLVHSNGNTYLHWYLQKPGQSPQL |
| NO: 213 | Humanized VL 1 | LIYKVSNRFSGVPDRFSGSGSGTDFTLKISRVEAEDVGVYYCSQSTHVPFTF |
| | | GCGTKLEIK |
| SEQ ID | VB 17 | DVVMTQSPLSLPVTLGQPASISCRSSQSLVHSNGNTYLHWFQQRPGQSPRR |
| NO: 214 | Humanized VL 2 | LIYKVSNRFSGVPDRFSGSGSGTDFTLKISRVEAEDVGVYYCSQSTHVPFTF |
| | | GCGTKLEIK |
| SEQ ID | VB 17 | DIVMTQTPLSLSVTPGQPASISCRSSQSLVHSNGNTYLHWYLQKPGQSPQLL |
| NO: 215 | Humanized VL 3 | IYKVSNRFSGVPDRFSGSGSGTDFTLKISRVEAEDVGVYYCSQSTHVPFTFG |
| | | CGTKLEIK |
| SEQ ID | VB 3 Humanized | EIVLTQSPAILSASLGERITLTCSANSTVGYIEIWYQQKPGQAPKLLIYTTSNL |
| NO: 216 | VL 1 | ASGVPSRFSGSGSGTDFSLTISSLEAEDFAVYFCHQWSFYPTFGCGTKLEIK |
| SEQ ID | VB 3 Humanized | EIVLTQSPATLSLSPGERATLSCSANSTVGYIEIWYQQKPGQAPRLLIYTTSN |
| NO: 217 | VL 2 | LASGIPARFSGSGSGTDFTLTISSLEPEDFAVYYCHQWSFYPTFGCGTKLEIK |
| SEQ ID | VB 3 Humanized | EIVLTQSPATLSLSPGERATLSCSANSTVGYIEIWYQQKPGQAPRLLIYTTSN |
| NO: 218 | VL 3 | LASGIPARFSGSGPGTDFTLTISSLEPEDFAVYYCHQWSFYPTFGCGTKLEIK |
| SEQ ID | VB 7.1 | DIVLTQSPVSLPVSPGERATISCRASKSVSTSGYSYMHWYQQKPGQPPKLLI |
| NO: 219 | Humanized VL 1 | YLASNLESGVPDRFSGSGSGTDFTLTISRVEAEDVAVYYCQHSRDLPWTFG |
| | | CGTKLEIK |
| SEQ ID | VB 7.1 | DIVLTQSPASLAVSPGQRATITCRASKSVSTSGYSYMHWYQQKPGQPPKLLI |
| NO: 220 | Humanized VL 2 | YLASNLESGVPARFSGSGSGTDFTLTINPVEANDTANYYCQHSRDLPWTFG |
| | | CGTKVEIK |
| SEQ ID | VB 7.1 | DIVMTQSPLSLPVTPGEPASISCRASKSVSTSGYSYMHWYLQKPGQSPQLLI |
| NO: 221 | Humanized VL 3 | YLASNLESGVPDRFSGSGSGTDFTLKISRVEAEDVGVYYCQHSRDLPWTFG |
| | | CGTKVEIK |
| SEQ ID | VB 7.1 | EIVLTQSPGTLSLSPGERATLSCRASKSVSTSGYSYMHWYQQKPGQPPRLLI |
| NO: 222 | Humanized VL 4 | YLASNLESGVPDRFSGSGSGTDFTLTISRLEAEDVAVYYCQHSRDLPWTFG |
| | | CGTKVEIK |
| SEQ ID | VB 7.1 | EIVLTQSPATLSLSPGERATLSCRASKSVSTSGYSYMHWYQQKPGQPPRLLI |
| NO: 223 | Humanized VL 5 | YLASNLESGVPDRFSGSGSGTDFTLTISRLEAEDVAVYYCQHSRDLPWTFG |
| | | CGTKVEIK |
| SEQ ID | VB 7.1 | DIVMTQSPDSLAVSLGERATINCRASKSVSTSGYSYMHWYQQKPGQPPKLL |
| NO: 224 | Humanized VL 6 | IYLASNLESGVPDRFSGSGSGTDFTLTISSLQAEDVAVYYCQHSRDLPWTFG |
| | | CGTKVEIK |
| SEQ ID | VB 5.3 | DIVLTQSPASLAVSPGERATISCRASESVDSSGNSFMHWYQQKPGQPPQLLI |
| NO: 225 | Humanized VL 1 | YRASNLESGVPDRFSGSGSGTDFTLTISRVEADDVAVYYCQQSFDDPFTFG |
| | | CGTKLEIK |
| SEQ ID | VB 5.3 | DIVLTQSPASLAVSPGQRATITCRASESVDSSGNSFMHWYQQKPGQPPKLLI |
| NO: 226 | Humanized VL 2 | YRASNLESGVPARFSGSGSGTDFTLTINPVEANDTANYYCQQSFDDPFTFG |
| | | CGTKLEIK |
| SEQ ID | VB 5.3 | EIVLTQSPATLSLSPGERATLSCRASESVDSSGNSFMHWYQQKPGQAPRLLI |
| NO: 227 | Humanized VL 3 | YRASNLESGIPARFSGSGSGTDFTLTISSLEPEDFAVYYCQQSFDDPFTFGCG |
| | | TKLEIK |
| SEQ ID | anti-VB 2 | QVQLVQSGAEVKKPGASVKVSCKASGFNIKSAYMHWVRQAPGQGLEWL |
| NO: 228 | Humanized VH 1 | GRIDPATGKTKYAPKFQGRATITADTSSNTAYMELSSLRSEDTAVYYCTRS |
| | | LNWDYGLDYWGQGTTVTVSS |
| SEQ ID | anti-VB 2 | QVQLVQSGAEVKKPGASVKVSCKASGFNIKSAYMHWVRQAPGQGLEWM |
| NO: 229 | Humanized VH 2 | GRIDPATGKTKYAPKFQGRVTMTADTSTSTAYMELSSLRSEDTAVYYCTRS |
| | | LNWDYGLDYWGQGTTVTVSS |
| SEQ ID | anti-VB 2 | QVQLQQSGPGLVKPSETLSLTCTASGFNIKSAYMHWVRQPPGKGLEWLGR |
| NO: 230 | Humanized VH 3 | IDPATGKTKYAPSLKSRATISADTSSNTASLKLSSVTAADTAVYYCTRSLN |
| | | WDYGLDYWGQGTTVTVSS |
| SEQ ID | anti-VB 2 | QVQLQESGPGLVKPSETLSLTCTASGFNIKSAYMHWIRQPPGKGLEWIGRID |
| NO: 231 | Humanized VH 4 | PATGKTKYAPSLKSRVTISADTSKNQASLKLSSVTAADTAVYYCTRSLNW |
| | | DYGLDYWGQGTTVTVSS |
| SEQ ID | anti-VB 2 | EVQLVQSGADLVRPGASLKLSCTASGFNIKSAYMHWVRQAPGQGPEWLG |
| NO: 232 | Humanized VH 5 | RIDPATGKTKYAPKFQGRVTITADTSSNTAYLQLSSLTSEDTAIYYCTRSLN |
| | | WDYGLDYWGQGTLVTVSS |
| SEQ ID | anti-VB 2 | QVQLVQSGAEVKKPGASVKVSCKASGFNIKSAYMHWVRQAPGQGLEWM |
| NO: 233 | Humanized VH 6 | GLVDPATGKTSYAQKFQGRVTITRDTSMSTAYMELSSLRSEDTAMYYCVR |
| | | SLNWDYGLDYWGQGTLVTVSS |
| SEQ ID | anti-VB 2 | QVQLVQSGAEVKKPGASVKVSCKASGFNIKSAYMHWVRQAPGQGLEWM |
| NO: 234 | Humanized VH 7 | GRIDPATGKTNYAQKFQGRVTMTRDTSISTAYMELSRLRSDDTAVYYCAR |
| | | SLNWDYGLDYWGQGTLVTVSS |
| SEQ ID | anti-VB 2 | QVQLVQSGAEVKKPGASVKVSCKASGFNIKSAAMHWVRQAPGQRLEWM |
| NO: 235 | Humanized VH 8 | GWIDPATGKTKYSQKFQGRVTITRDTSASTAYMELSSLRSEDTAVYYCARS |
| | | LNWDYGLDYWGQGTLVTVSS |
| SEQ ID | anti-VB 7.2 | QVHLVQSGAEVKRPGASVKVSCKASGYIFTDYYMHWVRQAPGQGLEWM |
| NO: 236 | Humanized VH 1 | GWINPSSGYTTYAQKFQDRVTLTADKSSSTAYMELSSLTSEDTAVYYCARS |
| | | LQLGRDYWGQGTLVTVSS |
| SEQ ID | anti-VB 7.2 | QVQLVQSGAEVKKPGASVKVSCKASGYIFTDYYMHWVRQAPGQGLEWM |
| NO: 237 | Humanized VH 2 | GWINPSSGYTNYAQKFQGRVTMTRDTSISTAYMELSRLRSDDTAVYYCAR |
| | | SLQLGRDYWGQGTLVTVSS |
| SEQ ID | anti-VB 7.2 | QVQLVQSGAEVKKPGASVKVSCKASGYIFTDYYMHWVRQAPGQGLEWM |
| NO: 238 | Humanized VH 3 | GIINPSSGYTSYAQKFQGRVTMTRDTSTSTVYMELSSLRSEDTAVYYCARS |
| | | LQLGRDYWGQGTLVTVSS |
| SEQ ID | anti-VB 4 | QVQLVQSGAEVKKPGASVKVSCKASGYTFTSYYIYWVRQAPGQGLEWLG |
| NO: 239 | Humanized VH 1 | WIYPGNGGTYYSEKFQGRVTFTADTSTNTAYMELSSLTSEDTAVYFCARGS |
| | | GDRYNSLAYWGQGTLVTVSS |
| SEQ ID | anti-VB 4 | QVQLVQSGAEVKKPGASVKVSCKASGYTFTSYAMHWVRQAPGQRLEWM |
| NO: 240 | Humanized VH 2 | GWIYPGNGGTKYSQKFQGRVTITRDTSASTAYMELSSLRSEDTAVYYCAR |
| | | GSGDRYNSLAYWGQGTLVTVSS |
| SEQ ID | anti-VB 4 | QVQLVQSGAEVKKPGASVKVSCKASGYTFTSYYMHWVRQAPGQGLEWM |
| NO: 241 | Humanized VH 3 | GIIYPGNGGTSYAQKFQGRVTMTRDTSTSTVYMELSSLRSEDTAVYYCARG |
| | | SGDRYNSLAYWGQGTLVTVSS |
| SEQ ID | anti-VB 4 | QVQLVQSGAEVKKPGASVKVSCKASGYTFTSYGISWVRQAPGQGLEWMG |
| NO: 242 | Humanized VH 4 | WIYPGNGGTNYAQKLQGRVTMTTDTSTSTAYMELRSLRSDDTAVYYCAR |
| | | GSGDRYNSLAYWGQGTLVTVSS |
| SEQ ID | anti-VB 4 | QVQLVQSGAEVKKPGASVKVSCKASGYTFTSYYMHWVRQAPGQGLEWM |
| NO: 243 | Humanized VH 5 | GWIYPGNGGTNYAQKFQGRVTMTRDTSISTAYMELSRLRSDDTAVYYCAR |
| | | GSGDRYNSLAYWGQGTLVTVSS |
| SEQ ID | anti-VB 13.2 | QVQLVQSGPEVKKPGASVKVSCKASGYTFTDYYIHWLRQAPGKGLEWIG |
| NO: 244 | Humanized VH 1 | WVNTNNGDTNYAQRFKGRVSLTRDKSTSTAYMELNSLTSEDTAVYYCAR |
| | | ALYTGSYWFAYWGQGTLVTVSS |
| SEQ ID | anti-VB 13.2 | QVQLVQSGAEVKKPGASVKVSCKASGYTFTDYYMHWVRQAPGQGLEWM |
| NO: 245 | Humanized VH 2 | GWINTNNGDTNYAQKFQGRVTMTRDTSISTAYMELSRLRSDDTAVYYCA |
| | | RALYTGSYWFAYWGQGTLVTVSS |
| SEQ ID | anti-VB 13.2 | QVQLVQSGAEVKKPGASVKVSCKASGYTFTDYYMHWVRQAPGQGLEWM |
| NO: 246 | Humanized VH 3 | GIINTNNGDTSYAQKFQGRVTMTRDTSTSTVYMELSSLRSEDTAVYYCAR |
| | | ALYTGSYWFAYWGQGTLVTVSS |
| SEQ ID | anti-VB 13.2 | QVQLVQSGAEVKKPGASVKVSCKASGYTFTDYYIHWLRQAPGQGLEWIGI |
| NO: 247 | Humanized VH 4 | VNTNNGDTNYNQKFQGRATLTVDKSSSTAYMELSSLRSEDTAVYYCARAL |
| | | YTGSYWFAYWGQGTLVTVSS |
| SEQ ID | anti-VB 13.2 | QVQLVQSGAEVKKPGASVKVSCKASGYTFTDYYIHWVRQAPGQGLEWM |
| NO: 248 | Humanized VH 5 | GIVNTNNGDTNYNQKFQGRVTMTVDKSTSTAYMELSSLRSEDTAVYYCA |
| | | RALYTGSYWFAYWGQGTLVTVSS |
| SEQ ID | anti-VB 13.2 | QVQLQQSGPGLVKPSETLSLTCTASGYTFTDYYIHWLRQPPGKGLEWIGIV |
| NO: 249 | Humanized VH 6 | NTNNGDTNYNQRLKSRATLSVDKSSSTASLKLSSVTAADTAVYYCARALY |
| | | TGSYWFAYWGQGTLVTVSS |
| SEQ ID | anti-VB 13.2 | QVQLQESGPGLVKPSETLSLTCTASGYTFTDYYIHWIRQPPGKGLEWIGIVN |
| NO: 250 | Humanized VH 7 | TNNGDTNYNQRLKSRVTISVDKSKNQASLKLSSVTAADTAVYYCARALYT |
| | | GSYWFAYWGQGTLVTVSS |
| SEQ ID | anti-VB 14 | QVQLVQSGAEVKKPGASVKLSCKASGFKTEDTYMHWVRQAPGQGLEWIG |
| NO: 251 | Humanized VH 1 | RIDPANGNTKYAPKFQDRVTITADTSANTAYLELSSLRSEDTAVYYCARGS |
| | | YYYAMDYWGQGTTVTVSS |
| SEQ ID | anti-VB 14 | QVQLVQSGAEVKKPGASVKVSCKASGFKTEDTAMHWVRQAPGQRLEWM |
| NO: 252 | Humanized VH 2 | GWIDPANGNTKYSQKFQGRVTITRDTSASTAYMELSSLRSEDTAVYYCAR |
| | | GSYYYAMDYWGQGTTVTVSS |
| SEQ ID | anti-VB 14 | QVQLVQSGAEVKKPGASVKVSCKASGFKTEDTYMHWVRQAPGQGLEWM |
| NO: 253 | Humanized VH 3 | GRIDPANGNTNYAQKFQGRVTMTRDTSISTAYMELSRLRSDDTAVYYCAR |
| | | GSYYYAMDYWGQGTTVTVSS |
| SEQ ID | anti-VB 14 | QVQLVQSGAEVKKPGASVKVSCKASGFKTEDTYMHWVRQAPGQGLEWM |
| NO: 254 | Humanized VH 4 | GIIDPANGNTSYAQKFQGRVTMTRDTSTSTVYMELSSLRSEDTAVYYCARG |
| | | SYYYAMDYWGQGTTVTVSS |
| SEQ ID | anti-VB 22 | EVQLVESGGGLVQPGGSLRLSCAASGFDFSRYWMNWVRQAPGKGLEWVS |
| NO: 255 | Humanized VH 1 | YINSDSNTINYADSVKGRFTISRDNAKNSLYLQMNSLRAEDTALYYCARGG |
| | | LLRDVWGQGTTVTVSS |
| SEQ ID | anti-VB 22 | EVQLVESGGGLVQPGGSLRLSCAASGFDFSRYSMNWVRQAPGKGLEWVS |
| NO: 256 | Humanized VH 2 | YINSDSNTIYYADSVKGRFTISRDNAKNSLYLQMNSLRAEDTAVYYCARG |
| | | GLLRDVWGQGTMVTVSS |
| SEQ ID | anti-VB 22 | EVQLVESGGGLVKPGGSLRLSCAASGFDFSRYSMNWVRQAPGKGLEWVS |
| NO: 257 | Humanized VH 3 | SINSDSNTIYYADSVKGRFTISRDNAKNSLYLQMNSLRAEDTAVYYCARGG |
| | | LLRDVWGQGTMVTVSS |
| SEQ ID | anti-VB 11 | QVQLVQSGPEVKRPGASVKISCKASGYRFTDSAMHWVRQAPGQGLEWMG |
| NO: 258 | Humanized VH 1 | WISSYNGNTNYAQKFQGRVTMTVDTSTSTAYMELSRLTSEDTAIYYCARS |
| | | RDAMDYWGQGTLVTVSS |
| SEQ ID | anti-VB 11 | QVQLVQSGAEVKKPGASVKVSCKASGYRFTDSGISWVRQAPGQGLEWMG |
| NO: 259 | Humanized VH 2 | WISSYNGNTNYAQKLQGRVTMTTDTSTSTAYMELRSLRSDDTAVYYCARS |
| | | RDAMDYWGQGTLVTVSS |
| SEQ ID | anti-VB 11 | QVQLVQSGAEVKKPGASVKVSCKASGYRFTDSYMHWVRQAPGQGLEWM |
| NO: 260 | Humanized VH 3 | GWISSYNGNTNYAQKFQGRVTMTRDTSISTAYMELSRLRSDDTAVYYCAR |
| | | SRDAMDYWGQGTLVTVSS |
| SEQ ID | anti-VB 1 | QVQLVQSGSELREPGASVKISCKASGYTFTDYYVHWVRQAPGKGLEWMG |
| NO: 261 | Humanized VH 1 | WINTYTGTPTYAQDFTGRFVFSLDTSASTAYLQISNLKAEDTAVYFCARSW |
| | | RRGIRGIGFDWGQGTLVTVSS |
| SEQ ID | anti-VB 1 | QVQLVQSGSELKKPGASVKVSCKASGYTFTDYAMNWVRQAPGQGLEWM |
| NO: 262 | Humanized VH 2 | GWINTYTGTPTYAQGFTGRFVFSLDTSVSTAYLQISSLKAEDTAVYYCARS |
| | | WRRGIRGIGFDWGQGTLVTVSS |
| SEQ ID | anti-VB 12 | EVQLVESGGGLVKPGGSLRLSCAASGFTFRSYGMSWVRQAPGKGLEWVA |
| NO: 263 | Humanized VH 1 | SISSGGSYTYYADSVKGRFTISRDNAKNSLYLQMSSLRAEDTAIYYCARHG |
| | | GNFFDYWGQGTLVTVSS |
| SEQ ID | anti-VB 12 | EVQLVESGGGLVKPGGSLRLSCAASGFTFRSYSMNWVRQAPGKGLEWVSS |
| NO: 264 | Humanized VH 2 | ISSGGSYIYYADSVKGRFTISRDNAKNSLYLQMNSLRAEDTAVYYCARHGG |
| | | NFFDYWGQGTLVTVSS |
| SEQ ID | anti-VB 12 | EVQLVESGGGLVQPGGSLRLSCAASGFTFRSYEMNWVRQAPGKGLEWVS |
| NO: 265 | Humanized VH 3 | YISSGGSYIYYADSVKGRFTISRDNAKNSLYLQMNSLRAEDTAVYYCARHG |
| | | GNFFDYWGQGTLVTVSS |
| SEQ ID | anti-VB 12 | QVQLVESGGGLVKPGGSLRLSCAASGFTFRSYYMSWIRQAPGKGLEWVSY |
| NO: 266 | Humanized VH 4 | ISSGGSYIYYADSVKGRFTISRDNAKNSLYLQMNSLRAEDTAVYYCARHGG |
| | | NFFDYWGQGTLVTVSS |
| SEQ ID | anti-VB 8 | EVQLVESGGGLVQPGGSLRLSCAASGFTFSNFGMHWVRQAPGKGLEWVA |
| NO: 267 | Humanized VH 1 | YISSGSSTIYYADSVKGRFTISRDNAKNTLFLQMNSLRAEDTAVYYCARRG |
| | | EGAMDYWGQGTLVTVSS |
| SEQ ID | anti-VB 8 | EVQLVESGGGLVQPGGSLRLSCAASGFTFSNFSMNWVRQAPGKGLEWVSY |
| NO: 268 | Humanized VH 2 | ISSGSSTIYYADSVKGRFTISRDNAKNSLYLQMNSLRAEDTAVYYCARRGE |
| | | GAMDYWGQGTLVTVSS |
| SEQ ID | anti-VB 8 | EVQLVESGGGLVKPGGSLRLSCAASGFTFSNFSMNWVRQAPGKGLEWVSY |
| NO: 269 | Humanized VH 3 | ISSGSSTIYYADSVKGRFTISRDNAKNSLYLQMNSLRAEDTAVYYCARRGE |
| | | GAMDYWGQGTLVTVSS |
| SEQ ID | anti-VB 13.1 | EVQLVESGGGLVKPGGSLRLSCAASGFTFRSYAMSWVRQAPGKGLEWVA |
| NO: 270 | Humanized VH 1 | SISNGGDYIYYADSVKGRFTISRDNARNTLYLQMSSLRAEDTAVYYCARPS |
| | | YYSDPWFFDVWGRGTTVTVSS |
| SEQ ID | anti-VB 13.1 | EVQLVESGGGLVKPGGSLRLSCAASGFTFRSYSMNWVRQAPGKGLEWVSS |
| NO: 271 | Humanized VH 2 | ISNGGDYIYYADSVKGRFTISRDNAKNSLYLQMNSLRAEDTAVYYCARPSY |
| | | YSDPWFFDVWGRGTLVTVSS |
| SEQ ID | anti-VB 13.1 | EVQLVESGGGLVQPGGSLRLSCAASGFTFRSYAMSWVRQAPGKGLEWVS |
| NO: 272 | Humanized VH 3 | AISNGGDYTYYADSVKGRFTISRDNSKNTLYLQMNSLRAEDTAVYYCAKP |
| | | SYYSDPWFFDVWGRGTLVTVSS |
| SEQ ID | anti-VB 13.1 | EVQLVESGGGLVQPGGSLRLSCAASGFTFRSYEMNWVRQAPGKGLEWVS |
| NO: 273 | Humanized VH 4 | YISNGGDYIYYADSVKGRFTISRDNAKNSLYLQMNSLRAEDTAVYYCARPS |
| | | YYSDPWFFDVWGRGTLVTVSS |
| SEQ ID | anti-VB 5.1 | QVQLVQSGPELKKPGASVRVSCKASGYTFTDYYIHWVRQAPGRGLEWVG |
| NO: 274 | Humanized VH 1 | WINPYNGRTGYAQKFKGRVTLTRDKSSSTAYMDLRSLTSEDTAVYYCAR |
| | | WDGSSYFDYWGQGTLVTVSS |
| SEQ ID | anti-VB 5.1 | QVQLVQSGAEVKKPGASVKVSCKASGYTFTDYYMHWVRQAPGQGLEWM |
| NO: 275 | Humanized VH 2 | GWINPYNGRTNYAQKFQGRVTMTRDTSISTAYMELSRLRSDDTAVYYCAR |
| | | WDGSSYFDYWGQGTLVTVSS |
| SEQ ID | anti-VB 5.1 | QVQLVQSGAEVKKPGASVKVSCKASGYTFTDYYMHWVRQAPGQGLEWM |
| NO: 276 | Humanized VH 3 | GIINPYNGRTSYAQKFQGRVTMTRDTSTSTVYMELSSLRSEDTAVYYCAR |
| | | WDGSSYFDYWGQGTLVTVSS |
| SEQ ID | anti-VB 5.1 | QVQLVQSGAEVKKPGASVKVSCKASGYTFTDYGISWVRQAPGQGLEWMG |
| NO: 277 | Humanized VH 4 | WINPYNGRTNYAQKLQGRVTMTTDTSTSTAYMELRSLRSDDTAVYYCAR |
| | | WDGSSYFDYWGQGTLVTVSS |
| SEQ ID | anti-VB 5.1 | QVQLVQSGAEVKKPGASVKVSCKASGYTFTDYYMHWVRQAPGQGLEWM |
| NO: 278 | Humanized VH 5 | GWINPYNGRTNYAQKFQGRVTMTRDTSISTAYMELSRLRSDDTAVYYCAR |
| | | WDGSSYFDYWGQGTLVTVSS |
| SEQ ID | anti-VB 9 | QVQLVQSGAELKRPGASVKLSCTASGFNIKNTFMHWVRQAPGQGLEWIGR |
| NO: 279 | Humanized VH 1 | IDPTNGYTKFAPKFQGRVTLTADTSSNTVYLQLSSLTSEDTAIYYCARDYD |
| | | APWFAYWGQGTLVIVSS |
| SEQ ID | anti-VB 9 | QVQLVQSGAEVKKPGASVKVSCKASGFNIKNTYMHWVRQAPGQGLEWM |
| NO: 280 | Humanized VH 2 | GIIDPTNGYTSYAQKFQGRVTMTRDTSTSTVYMELSSLRSEDTAVYYCARD |
| | | YDAPWFAYWGQGTLVTVSS |
| SEQ ID | anti-VB 9 | QVQLVQSGAEVKKPGASVKVSCKASGFNIKNTYMHWVRQAPGQGLEWM |
| NO: 281 | Humanized VH 3 | GRIDPTNGYTNYAQKFQGRVTMTRDTSISTAYMELSRLRSDDTAVYYCAR |
| | | DYDAPWFAYWGQGTLVTVSS |
| SEQ ID | anti-VB 9 | QVQLVQSGAEVKKPGASVKVSCKASGFNIKNTAMHWVRQAPGQRLEWM |
| NO: 282 | Humanized VH 4 | GWIDPTNGYTKYSQKFQGRVTITRDTSASTAYMELSSLRSEDTAVYYCAR |
| | | DYDAPWFAYWGQGTLVTVSS |
| SEQ ID | anti-VB 17 | QVQLQESGPGLVKPSQTLSLTCSVSGYSITSGYFWNWIRQPPGKGLEWIGYI |
| NO: 283 | Humanized VH 1 | SYDGSTNYNPSLKSRISISVDTSKNQFSLKLNSVTAADTAVYYCASPSPGTG |
| | | YAVDYWGQGTLVTVSS |
| SEQ ID | anti-VB 17 | QVQLQESGPGLVKPSETLSLTCTVSGYSITSGYWSWIRQPPGKGLEWIGYIS |
| NO: 284 | Humanized VH 2 | YDGSTNYNPSLKSRVTISVDTSKNQFSLKLSSVTAADTAVYYCARPSPGTG |
| | | YAVDYWGQGTLVTVSS |
| SEQ ID | anti-VB 17 | QVQLQESGPGLVKPSQTLSLTCAVSGYSITSGYYWGWIRQPPGKGLEWIGY |
| NO: 285 | Humanized VH 3 | ISYDGSTYYNPSLKSRVTISVDTSKNQFSLKLSSVTAADTAVYYCARPSPGT |
| | | GYAVDYWGQGTLVTVSS |
| SEQ ID | anti-VB 3 | QVQLVQSGPEVKKPGASVKISCKASGYTFSGSWMHWVRQAPGKGLEWIG |
| NO: 286 | Humanized VH 1 | RIYPGDGDTDYSQKFQGRVTLTADKSSSTAYMELSSLTSEDTAVYFCARSG |
| | | YFNYVPVFDYWGQGTLVTVSS |
| SEQ ID | anti-VB 3 | QVQLVQSGAEVKKPGASVKVSCKASGYTFSGSYMHWVRQAPGQGLEWM |
| NO: 287 | Humanized VH 2 | GRIYPGDGDTNYAQKFQGRVTMTRDTSISTAYMELSRLRSDDTAVYYCAR |
| | | SGYFNYVPVFDYWGQGTLVTVSS |
| SEQ ID | anti-VB 3 | QVQLVQSGAEVKKPGASVKVSCKASGYTFSGSAMHWVRQAPGQRLEWM |
| NO: 289 | Humanized VH 3 | GWIYPGDGDTKYSQKFQGRVTITRDTSASTAYMELSSLRSEDTAVYYCARS |
| | | GYFNYVPVFDYWGQGTLVTVSS |
| SEQ ID | anti-VB 7.1 | EVQLVESGGGLVKPGGSLRLSCAASGFTFSDYYMNWVRQTPGKGLEWVA |
| NO: 290 | Humanized VH 1 | SISGGGSYTYYADSVKGRFTISRDNAKNSLYLQMSSLRAEDTAVYFCARER |
| | | DIYYGNFNAMVYWGQGTLVTVSS |
| SEQ ID | anti-VB 7.1 | EVQLVESGGGLVKPGGSLRLSCAASGFTFSDYYMNWVRQAPGKGLEWVS |
| NO: 291 | Humanized VH 2 | SISGGGSYIYYADSVKGRFTISRDNAKNSLYLQMNSLRAEDTAVYYCARER |
| | | DIYYGNFNAMVYWGRGTLVTVSS |
| SEQ ID | anti-VB 7.1 | EVQLVESGGGLVKPGGSLRLSCAASGFTFSDYSMNWVRQAPGKGLEWVSS |
| NO: 292 | Humanized VH 3 | ISGGGSYIYYADSVKGRFTISRDNAKNSLYLQMNSLRAEDTAVYYCARERD |
| | | IYYGNFNAMVYWGRGTLVTVSS |
| SEQ ID | anti-VB 7.1 | QVQLVESGGGLVKPGGSLRLSCAASGFTFSDYYMSWIRQAPGKGLEWVSY |
| NO: 293 | Humanized VH 4 | ISGGGSYIYYADSVKGRFTISRDNAKNSLYLQMNSLRAEDTAVYYCARERD |
| | | IYYGNFNAMVYWGQGTLVTVSS |
| SEQ ID | anti-VB 5.3 | QVQLVQSGPELKKPGASVKISCKASGYAFSSSWMHWVRQAPGQGLEWIG |
| NO: 294 | Humanized VH 1 | RIYPGDGDTKYAQKFQGRVTLTADKSSSTAYMELSSLTSEDTAVYFCARR |
| | | GTGGWYFDVWGKGTLVTVSS |
| SEQ ID | anti-VB 5.3 | QVQLVQSGAEVKKPGASVKVSCKASGYAFSSSAMHWVRQAPGQRLEWM |
| NO: 295 | Humanized VH 2 | GWIYPGDGDTKYSQKFQGRVTITRDTSASTAYMELSSLRSEDTAVYYCAR |
| | | RGTGGWYFDVWGRGTLVTVSS |
| SEQ ID | anti-VB 2 | DIVLTQSPDSLAVSLGERATINCKASKSVSILGTHLIHWYQQKPGQPPKLLIYA |
| NO: 296 | Humanized VL 1 | ASNLESGVPDRFSGSGSETVFTLTISSLQAEDVATYFCQQSIEDPWTFGGGT |
| | | KLEIK |
| SEQ ID | anti-VB 2 | DIVLTQSPDSLAVSLGERATINCKASKSVSILGTHLIHWYQQKPGQPPKLLIYA |
| NO: 297 | Humanized VL 2 | ASNRESGVPDRFSGSGSETDFTLTISSLQAEDVAVYYCQQSIEDPWTFGGGT |
| | | KLEIK |
| SEQ ID | anti-VB 2 | DIQLTQSPSSLSASVGDRVTITCRASKSVSILGTHLIHWYQQKPGKAPKLLIYA |
| NO: 298 | Humanized VL 3 | ASNLESGVPSRFSGSGSETVFTLTISSLQPEDFATYFCQQSIEDPWTFGGGTK |
| | | LEIK |
| SEQ ID | anti-VB 2 | DIQLTQSPSSLSASVGDRVTITCRASKSVSILGTHLIHWYQQKPGKAPKLLIYA |
| NO: 299 | Humanized VL 4 | ASNLESGVPSRFSGSGSETDFTLTISSLQPEDFATYYCQQSIEDPWTFGGGTK |
| | | LEIK |
| SEQ ID | anti-VB 2 | DIVLTQSPASLAVSLGERATISCRASKSVSILGTHLIHWYQQKPGQPPKLLIYA |
| NO: 300 | Humanized VL 5 | ASNLESGVPDRFSGSGSGTDFTLTISRVEAEDVAVYYCQQSIEDPWTFGGGT |
| | | KLEIK |
| SEQ ID | anti-VB 2 | DIVLTQSPASLAVSPGERATISCRASKSVSILGTHLIHWYQQKPGQPPKLLIYA |
| NO: 301 | Humanized VL 6 | ASNLESGVPDRFSGSGSGTDFTLTISRVEAEDVAVYYCQQSIEDPWTFGQGT |
| | | KVEIK |
| SEQ ID | anti-VB 2 | DIVMTQSPDSLAVSLGERATINCRASKSVSILGTHLIHWYQQKPGQPPKLLIY |
| NO: 302 | Humanized VL 7 | AASNLESGVPDRFSGSGSGTDFTLTISSLQAEDVAVYYCQQSIEDPWTFGQG |
| | | TKVEIK |
| SEQ ID | anti-VB 2 | EIVLTQSPATLSLSPGERATLSCRASKSVSILGTHLIHWYQQKPGQSPRLLIYAA |
| NO: 303 | Humanized VL 8 | SNLESGVPDRFSGSGSGTDFTLTISRLEAEDVAVYYCQQSIEDPWTFGQGTK |
| | | VEIK |
| SEQ ID | anti-VB 7.2 | DIQMTQSPSFLSASVGDRVTITCKASQDVYTAVAWYQQKPGQAPKLLIYSA |
| NO: 304 | Humanized VL 1 | SNRYTGVPSRFSGSGSGTDFTLTISSLQAEDLATYYCQQHYTTPRTFGQGTK |
| | | VEIK |
| SEQ ID | anti-VB 7.2 | DIQMTQSPSSLSASVGDRVTITCKASQDVYTAVAWYQQKPGKAPKLLIYSA |
| NO: 305 | Humanized VL 2 | SNRYTGVPSRFSGSGSGTDFTFTISSLQPEDIATYYCQQHYTTPRTFGQGTK |
| | | LEIK |
| SEQ ID | anti-VB 7.2 | DIQMTQSPSSVSASVGDRVTITCKASQDVYTAVAWYQQKPGKAPKLLIYS |
| NO: 306 | Humanized VL 3 | ASNRYTGVPSRFSGSGSGTDFTLTISSLQPEDFATYYCQQHYTTPRTFGQGT |
| | | KLEIK |
| SEQ ID | anti-VB 7.2 | AIQLTQSPSSLSASVGDRVTITCKASQDVYTAVAWYQQKPGKAPKLLIYSA |
| NO: 307 | Humanized VL 4 | SNRYTGVPSRFSGSGSGTDFTLTISSLQPEDFATYYCQQHYTTPRTFGQGTK |
| | | LEIK |
| SEQ ID | anti-VB 4 | DIQMTQSPSSVSASVGDRVTITCLASEGISNFLAWYQQKPGKAPKLLIYYTS |
| NO: 308 | Humanized VL 1 | SLQDGVPSRFSGSGSGTDFTLTISSLQPEDFATYYCQQGYKFPRTFGQGTKL |
| | | EIK |
| SEQ ID | anti-VB 4 | DIQMTQSPSSLSASVGDRVTITCLASEGISNFLAWFQQKPGKAPKSLIYYTSS |
| NO: 309 | Humanized VL 2 | LQDGVPSRFSGSGSGTDFTLTISSLQPEDFATYYCQQGYKFPRTFGQGTKLE |
| | | IK |
| SEQ ID | anti-VB 13.2 | EIVLTQSPATLSVSPGEKVTITCSASSSISSTNLHWYQQKPGQSPRLLIYGTS |
| NO: 310 | Humanized VL 1 | NLASGVPDRFSGSGSGTDFTLTISSLEAEDFATYYCQQWSRYPLTFGQGTK |
| | | LEIK |
| SEQ ID | anti-VB 13.2 | EIVLTQSPATLSLSPGERATLSCSASSSISSTNLHWYQQKPGQAPRLLIYGTS |
| NO: 311 | Humanized VL 2 | NLASGIPDRFSGSGSGTDFTLTISSLEPEDFAVYYCQQWSRYPLTFGQGTKL |
| | | EIK |
| SEQ ID | anti-VB 13.2 | EIVLTQSPATLSLSPGERATLSCSASSSISSTNLHWYQQKPGQAPRLLIYGTS |
| NO: 312 | Humanized VL 3 | NLASGIPDRFSGSGSGTDFTLTISRLEPEDFAVYYCQQWSRYPLTFGQGTKL |
| | | EIK |
| SEQ ID | anti-VB 13.2 | EIVLTQSPGTLSLSPGERATLSCSASSSISSTNLHWYQQKPGQAPRLLIYGTS |
| NO: 313 | Humanized VL 4 | NLASGIPDRFSGSGSGTDFTLTISRLEPEDFAVYYCQQWSRYPLTFGQGTKL |
| | | EIK |
| SEQ ID | anti-VB 13.2 | EIQLTQSPSSLSASVGDRVTITCRASSSISSTNLHWYQQKPGKAPKPWIYGTS |
| NO: 314 | Humanized VL 5 | NLASGVPSRFSGSGSGTSYSLTISSLQPEDFATYYCQQWSRYPLTFGGGTKL |
| | | EIK |
| SEQ ID | anti-VB 13.2 | AIQLTQSPSSLSASVGDRVTITCRASSSISSTNLHWYQQKPGKAPKPLIYGTS |
| NO: 315 | Humanized VL 6 | NLASGVPSRFSGSGSGTDYTLTISSLQPEDFATYYCQQWSRYPLTFGGGTKL |
| | | EIK |
| SEQ ID | anti-VB 13.2 | EIVLTQSPATLSLSPGERATLSCRASSSISSTNLHWYQQKPGQAPRPWIYGTS |
| NO: 316 | Humanized VL 7 | NLASGVPARFSGSGSGTSYSLTISSLEPEDFATYYCQQWSRYPLTFGGGTKL |
| | | EIK |
| SEQ ID | anti-VB 13.2 | EIVLTQSPATLSLSPGERATLSCRASSSISSTNLHWYQQKPGQAPRPLIYGTS |
| NO: 317 | Humanized VL 8 | NRATGIPARFSGSGSGTDYTLTISSLEPEDFAVYYCQQWSRYPLTFGGGTKL |
| | | EIK |
| SEQ ID | anti-VB 14 | DIVLTQSPASLAVSPGERATISCRASESVDSYGNSFMHWYQQKPGQPPKLLI |
| NO: 318 | Humanized VL 1 | YRASNLESGVPDRFSGSGSGTDFTLTISRVEADDVAVYYCQQSNEDPYTFG |
| | | QGTKLEIK |
| SEQ ID | anti-VB 14 | DIVLTQSPASLAVSPGERATISCRASESVDSYGNSFMHWYQQKPGQPPKLLI |
| NO: 319 | Humanized VL 2 | YRASNLESGVPDRFSGSGSGTDFTLTISRVEAEDVAVYYCQQSNEDPYTFG |
| | | QGTKLEIK |
| SEQ ID | anti-VB 14 | DIVMTQSPDSLAVSLGERATINCRASESVDSYGNSFMHWYQQKPGQPPKLL |
| NO: 320 | Humanized VL 3 | IYRASNLESGVPDRFSGSGSGTDFTLTISSLQAEDVAVYYCQQSNEDPYTFG |
| | | QGTKLEIK |
| SEQ ID | anti-VB 14 | EIVLTQSPATLSLSPGERATLSCRASESVDSYGNSFMHWYQQKPGQPPRLLI |
| NO: 321 | Humanized VL 4 | YRASNLESGVPDRFSGSGSGTDFTLTISRLEAEDVAVYYCQQSNEDPYTFG |
| | | QGTKLEIK |
| SEQ ID | anti-VB 14 | EIVLTQSPATLSLSPGERATLSCRASESVDSYGNSFMHWYQQKPGQPPRLLI |
| NO: 322 | Humanized VL 5 | YRASNLESGVPDRFSGSGSGTDFTLTISSLEAEDFAVYYCQQSNEDPYTFGQ |
| | | GTKLEIK |
| SEQ ID | anti-VB 14 | EIVLTQSPGTLSLSPGERATLSCRASESVDSYGNSFMHWYQQKPGQPPRLLI |
| NO: 323 | Humanized VL 6 | YRASNLESGVPDRFSGSGSGTDFTLTISRLEAEDVAVYYCQQSNEDPYTFG |
| | | QGTKLEIK |
| SEQ ID | anti-VB 22 | QAVVTQEPSLTVSPGGTVTLTCRSSTGAVTTSNYANWFQQKPGHAPRTLIY |
| NO: 324 | Humanized VL 1 | GTNNRAPGVPARFSGSLLGDKAALTITGAQTEDEADYFCALWYSNHWVFG |
| | | GGTKLTVL |
| SEQ ID | anti-VB 22 | QAVVTQEPSLTVSPGGTVTLTCRSSTGAVTTSNYANWFQQKPGQAPRTLIY |
| NO: 325 | Humanized VL 2 | GTNNRAPWTPARFSGSLLGGKAALTLSGAQPEDEAEYYCALWYSNHWVF |
| | | GGGTKLTVL |
| SEQ ID | anti-VB 22 | QTVVTQEPSLTVSPGGTVTLTCRSSTGAVTTSNYANWFQQKPGQAPRALIY |
| NO: 326 | Humanized VL 3 | GTNNRAPWTPARFSGSLLGGKAALTLSGVQPEDEAEYYCALWYSNHWVF |
| | | GGGTKLTVL |
| SEQ ID | anti-VB 22 | QTVVTQEPSFSVSPGGTVTLTCRSSTGAVTTSNYANWYQQTPGQAPRTLIY |
| NO: 327 | Humanized VL 4 | GTNNRAPGVPDRFSGSILGNKAALTITGAQADDESDYYCALWYSNHWVFG |
| | | GGTKLTVL |
| SEQ ID | anti-VB 11 | DIQMTQSPSSLSASVGDRVTITCKASQDINSYLSWFQQKPGKAPKLLIYRAN |
| NO: 328 | Humanized VL 1 | RLVDGVPSRFSGSGSGTDYTLTISSLQPEDFATYYCLQYDEFPFTFGQGTRL |
| | | EIK |
| SEQ ID | anti-VB 11 | DIQMTQSPSSLSASVGDRVTITCKASQDINSYLSWFQQKPGKAPKSLIYRAN |
| NO: 329 | Humanized VL 2 | RLVDGVPSRFSGSGSGTDFTLTISSLQPEDFATYYCLQYDEFPFTFGQGTRL |
| | | EIK |
| SEQ ID | anti-VB 11 | DIQMTQSPSSLSASVGDRVTITCKASQDINSYLSWYQQKPGKAPKLLIYRA |
| NO: 330 | Humanized VL 3 | NRLVDGVPSRFSGSGSGTDFTFTISSLQPEDIATYYCLQYDEFPFTFGQGTRL |
| | | EIK |
| SEQ ID | anti-VB 11 | DIQMTQSPSSLSASVGDRVTITCKASQDINSYLSWYQQKPGKAPKLLLYRA |
| NO: 331 | Humanized VL 4 | NRLVDGVPSRFSGSGSGTDYTLTISSLQPEDFATYYCLQYDEFPFTFGQGTR |
| | | LEIK |
| SEQ ID | anti-VB 1 | DVQMTQSPSTLSASVGESVTITCKASKSINKYLAWYQQKPGKAPKLLIYDG |
| NO: 332 | Humanized VL 1 | STLQSGVPSRFSGSGSGTDFTLTISGLQPEDFATYYCQQHNEYPPTFGQGTK |
| | | LEIK |
| SEQ ID | anti-VB 1 | DIQMTQSPSSLSASVGDRVTITCKASKSINKYLAWYQQKPGKAPKLLIYDG |
| NO: 333 | Humanized VL 2 | STLQSGVPSRFSGSGSGTDFTLTISSLQPEDFATYYCQQHNEYPPTFGQGTK |
| | | LEIK |
| SEQ ID | anti-VB 1 | DIQMTQSPSTLSASVGDRVTITCKASKSINKYLAWYQQKPGKAPKLLIYDG |
| NO: 334 | Humanized VL 3 | STLQSGVPSRFSGSGSGTEFTLTISSLQPDDFATYYCQQHNEYPPTFGQGTK |
| | | LEIK |
| SEQ ID | anti-VB 1 | DIQLTQSPSFLSASVGDRVTITCKASKSINKYLAWYQQKPGKAPKLLIYDGS |
| NO: 335 | Humanized VL 4 | TLQSGVPSRFSGSGSGTEFTLTISSLQPEDFATYYCQQHNEYPPTFGQGTKL |
| | | EIK |
| SEQ ID | anti-VB 1 | DIQMTQSPSSLSASVGDRVTITCKASKSINKYLAWFQQKPGKAPKSLIYDGS |
| NO: 336 | Humanized VL 5 | TLQSGVPSRFSGSGSGTDFTLTISSLQPEDFATYYCQQHNEYPPTFGQGTKL |
| | | EIK |
| SEQ ID | anti-VB 12 | EIVLTQSPSFLSASPGEKVTITCSVSSSVSYMEIWYQQKPGQAPKLLIYDTSK |
| NO: 337 | Humanized VL 1 | LASGVPARFSGSGSGTDFTLTISSLEAEDFATYYCQQWSSNPQYTFGQGTK |
| | | LEIK |
| SEQ ID | anti-VB 12 | AIQLTQSPSSLSASVGDRVTITCSVSSSVSYMEIWYQQKPGKAPKLLIYDTS |
| NO: 338 | Humanized VL 2 | KLASGVPSRFSGSGSGTDFTLTISSLQPEDFATYYCQQWSSNPQYTFGQGTK |
| | | LEIK |
| SEQ ID | anti-VB 12 | EIVLTQSPATLSLSPGERATLSCSVSSSVSYMEIWYQQKPGQAPRLLIYDTSK |
| NO: 339 | Humanized VL 3 | LASGIPARFSGSGSGTDFTLTISSLEPEDFAVYYCQQWSSNPQYTFGQGTKL |
| | | EIK |
| SEQ ID | anti-VB 12 | DIQMTQSPSSLSASVGDRVTITCSVSSSVSYMEIWYQQKPGKAPKLLIYDTS |
| NO: 340 | Humanized VL 4 | KLASGVPSRFSGSGSGTDFTLTISSLQPEDFATYYCQQWSSNPQYTFGQGTK |
| | | LEIK |
| SEQ ID | anti-VB 12 | DIQLTQSPSFLSASVGDRVTITCSVSSSVSYMEIWYQQKPGKAPKLLIYDTS |
| NO: 341 | Humanized VL 5 | KLASGVPSRFSGSGSGTEFTLTISSLQPEDFATYYCQQWSSNPQYTFGQGTK |
| | | LEIK |
| SEQ ID | anti-VB 8 | EIVLTQSPAILSVSLGEKVTLSCRASSSVNYIYWYQQKPGKAPKLLIYYTSN |
| NO: 342 | Humanized VL 1 | LAPGVPTRFSGSGSGTDFSLTISSLQPEDAATYYCQQFTSSPFTFGQGTKLEI |
| | | K |
| SEQ ID | anti-VB 8 | EIVLTQSPATLSLSPGERATLSCRASSSVNYIYWYQQKPGQAPRLLIYYTSN |
| NO: 343 | Humanized VL 2 | LAPGIPARFSGSGSGTDFTLTISSLEPEDFAVYYCQQFTSSPFTFGQGTKLEIK |
| SEQ ID | anti-VB 8 | DIQLTQSPSSLSASVGDRVTITCRASSSVNYIYWYQQKPGKAPKLLIYYTSN |
| NO: 344 | Humanized VL 3 | LAPGVPSRFSGSGSGTDFTLTISSLQPEDFATYYCQQFTSSPFTFGQGTKLEI |
| | | K |
| SEQ ID | anti-VB 13.1 | EIVLTQSPAILSASPGEKVTLTCSAGSSVSFMEIWYQQKPGQSPKLLIYDTSK |
| NO: 345 | Humanized VL 1 | LASGVPGRFSGSGSGTDFSLTISRLEPDDAAYYYLQGSGFPLTFGQGTKLEI |
| | | K |
| SEQ ID | anti-VB 13.1 | EIVLTQSPATLSLSPGERATLSCSAGSSVSFMEIWYQQKPGQAPRLLIYDTSK |
| NO: 346 | Humanized VL 2 | LASGIPARFSGSGSGTDFTLTISSLEPEDFAVYYCLQGSGFPLTFGQGTKLEI |
| | | K |
| SEQ ID | anti-VB 13.1 | EIVLTQSPATLSLSPGERATLSCSAGSSVSFMEIWYQQKPGQAPRLLIYDTSK |
| NO: 347 | Humanized VL 3 | LASGIPDRFSGSGSGTDFTLTISRLEPEDFAVYYCLQGSGFPLTFGQGTKLEI |
| | | K |
| SEQ ID | anti-VB 5.1 | EIVLTQSPAILAVSLGEKVTITCSASSSVSYMHWYQQKPGKAPKLLIYEISKL |
| NO: 348 | Humanized VL 1 | ASGVPARFSGSGSGTSFSLTISSLQAEDAATYYCQQWNYPLLTFGQGTKLEI |
| | | K |
| SEQ ID | anti-VB 5.1 | DIQLTQSPSFLSASVGDRVTITCSASSSVSYMHWYQQKPGKAPKLLIYEISK |
| NO: 349 | Humanized VL 2 | LASGVPSRFSGSGSGTEFTLTISSLQPEDFATYYCQQWNYPLLTFGQGTKLE |
| | | IK |
| SEQ ID | anti-VB 5.1 | DIQLTQSPSSLSASVGDRVTITCSASSSVSYMHWYQQKPGKAPKLLIYEISK |
| NO: 350 | Humanized VL 3 | LASGVPSRFSGSGSGTDFTLTISSLQPEDFATYYCQQWNYPLLTFGQGTKLE |
| | | IK |
| SEQ ID | anti-VB 9 | ETVMTQSPASLSVAPGEKVTITCISSTDIDDDMNWYQQKPGEPPKLLISEGN |
| NO: 351 | Humanized VL 1 | TLRPGVPSRFSGSGYGTDFTFTINNLQSEDVADYYCLQSDNMPLTFGQGTK |
| | | LEIK |
| SEQ ID | anti-VB 9 | ETTLTQSPAFMSATPGDKVNISCISSTDIDDDMNWYQQKPGEAPIFIIQEGNT |
| NO: 352 | Humanized VL 2 | LRPGIPPRFSGSGYGTDFTLTINNIESEDAAYYFCLQSDNMPLTFGQGTKLEI |
| | | K |
| SEQ ID | anti-VB 9 | DIQMTQSPSSLSASVGDRVTITCISSTDIDDDMNWYQQKPGKAPKLLIYEGN |
| NO: 353 | Humanized VL 3 | TLRPGVPSRFSGSGSGTDFTFTISSLQPEDIATYYCLQSDNMPLTFGQGTKLE |
| | | IK |
| SEQ ID | anti-VB 9 | DVVMTQSPAFLSVTPGEKVTITCISSTDIDDDMNWYQQKPGQAPKLLIKEG |
| NO: 354 | Humanized VL 4 | NTLRPGVPSRFSGSGSGTDFTLTISSLEAEDAATYYCLQSDNMPLTFGQGTK |
| | | LEIK |
| SEQ ID | anti-VB 17 | DVVMTQTPLSLPVTLGQPASISCRSSQSLVHSNGNTYLHWYLQKPGQSPQL |
| NO: 355 | Humanized VL 1 | LIYKVSNRFSGVPDRFSGSGSGTDFTLKISRVEAEDVGVYYCSQSTHVPFTF |
| | | GQGTKLEIK |
| SEQ ID | anti-VB 17 | DVVMTQSPLSLPVTLGQPASISCRSSQSLVHSNGNTYLHWFQQRPGQSPRR |
| NO: 356 | Humanized VL 2 | LIYKVSNRFSGVPDRFSGSGSGTDFTLKISRVEAEDVGVYYCSQSTHVPFTF |
| | | GQGTKLEIK |
| SEQ ID | anti-VB 17 | DIVMTQTPLSLSVTPGQPASISCRSSQSLVHSNGNTYLHWYLQKPGQSPQLL |
| NO: 357 | Humanized VL 3 | IYKVSNRFSGVPDRFSGSGSGTDFTLKISRVEAEDVGVYYCSQSTHVPFTFG |
| | | QGTKLEIK |
| SEQ ID | anti-VB 3 | EIVLTQSPAILSASLGERITLTCSANSTVGYIEIWYQQKPGQAPKLLIYTTSNL |
| NO: 358 | Humanized VL 1 | ASGVPSRFSGSGSGTDFSLTISSLEAEDFAVYFCHQWSFYPTFGQGTKLEIK |
| SEQ ID | anti-VB 3 | EIVLTQSPATLSLSPGERATLSCSANSTVGYIEIWYQQKPGQAPRLLIYTTSN |
| NO: 359 | Humanized VL 2 | LASGIPARFSGSGSGTDFTLTISSLEPEDFAVYYCHQWSFYPTFGQGTKLEIK |
| SEQ ID | anti-VB 3 | EIVLTQSPATLSLSPGERATLSCSANSTVGYIEIWYQQKPGQAPRLLIYTTSN |
| NO: 360 | Humanized VL 3 | LASGIPARFSGSGPGTDFTLTISSLEPEDFAVYYCHQWSFYPTFGQGTKLEIK |
| SEQ ID | anti-VB 7.1 | DIVLTQSPVSLPVSPGERATISCRASKSVSTSGYSYMHWYQQKPGQPPKLLI |
| NO: 361 | Humanized VL 1 | YLASNLESGVPDRFSGSGSGTDFTLTISRVEAEDVAVYYCQHSRDLPWTFG |
| | | GGTKLEIK |
| SEQ ID | anti-VB 7.1 | DIVLTQSPASLAVSPGQRATITCRASKSVSTSGYSYMHWYQQKPGQPPKLLI |
| NO: 362 | Humanized VL 2 | YLASNLESGVPARFSGSGSGTDFTLTINPVEANDTANYYCQHSRDLPWTFG |
| | | QGTKVEIK |
| SEQ ID | anti-VB 7.1 | DIVMTQSPLSLPVTPGEPASISCRASKSVSTSGYSYMHWYLQKPGQSPQLLI |
| NO: 363 | Humanized VL 3 | YLASNLESGVPDRFSGSGSGTDFTLKISRVEAEDVGVYYCQHSRDLPWTFG |
| | | QGTKVEIK |
| SEQ ID | anti-VB 7.1 | EIVLTQSPGTLSLSPGERATLSCRASKSVSTSGYSYMHWYQQKPGQPPRLLI |
| NO: 364 | Humanized VL 4 | YLASNLESGVPDRFSGSGSGTDFTLTISRLEAEDVAVYYCQHSRDLPWTFG |
| | | QGTKVEIK |
| SEQ ID | anti-VB 7.1 | EIVLTQSPATLSLSPGERATLSCRASKSVSTSGYSYMHWYQQKPGQPPRLLI |
| NO: 365 | Humanized VL 5 | YLASNLESGVPDRFSGSGSGTDFTLTISRLEAEDVAVYYCQHSRDLPWTFG |
| | | QGTKVEIK |
| SEQ ID | anti-VB 7.1 | DIVMTQSPDSLAVSLGERATINCRASKSVSTSGYSYMHWYQQKPGQPPKLL |
| NO: 366 | Humanized VL 6 | IYLASNLESGVPDRFSGSGSGTDFTLTISSLQAEDVAVYYCQHSRDLPWTFG |
| | | QGTKVEIK |
| SEQ ID | anti-VB 5.3 | DIVLTQSPASLAVSPGERATISCRASESVDSSGNSFMHWYQQKPGQPPQLLI |
| NO: 367 | Humanized VL 1 | YRASNLESGVPDRFSGSGSGTDFTLTISRVEADDVAVYYCQQSFDDPFTFG |
| | | QGTKLEIK |
| SEQ ID | anti-VB 5.3 | DIVLTQSPASLAVSPGQRATITCRASESVDSSGNSFMHWYQQKPGQPPKLLI |
| NO: 368 | Humanized VL 2 | YRASNLESGVPARFSGSGSGTDFTLTINPVEANDTANYYCQQSFDDPFTFG |
| | | QGTKLEIK |
| SEQ ID | anti-VB 5.3 | EIVLTQSPATLSLSPGERATLSCRASESVDSSGNSFMHWYQQKPGQAPRLLI |
| NO: 369 | Humanized VL 3 | YRASNLESGIPARFSGSGSGTDFTLTISSLEPEDFAVYYCQQSFDDPFTFGQG |
| | | TKLEIK |
| SEQ ID | VB7.2 VH CDR1 | DYTMH |
| NO: 370 | | |
| SEQ ID | VB7.2 VH CDR2 | HINPSSGYSTYNQKFKD |
| NO: 371 | | |
| SEQ ID | VB7.2 VH CDR3 | SLQLGRDY |
| NO: 372 | | |
| SEQ ID | VB4 VH CDR1 | SYYIY |
| NO: 373 | | |
| SEQ ID | VB4 VH CDR2 | YIYPGNGGTYYSEKFKG |
| NO: 374 | | |
| SEQ ID | VB4 VH CDR3 | GSGDRYNSLAY |
| NO: 375 | | |
| SEQ ID | VB13.2 VH | DYYIH |
| NO: 376 | CDR1 | |
| SEQ ID | VB13.2 VH | IVNTNNGDTNYNQRFKG |
| NO: 377 | CDR2 | |
| SEQ ID | VB13.2 VH | ALYTGSYWFAY |
| NO: 378 | CDR3 | |
| SEQ ID | VB14 VH CDR1 | GFKTEDT |
| NO: 379 | | |
| SEQ ID | VB14 VH CDR2 | DPANGN |
| NO: 380 | | |
| SEQ ID | VB14 VH CDR3 | GSYYYAMDY |
| NO: 381 | | |
| SEQ ID | VB22 VH CDR1 | RYWMN |
| NO: 382 | | |
| SEQ ID | VB22 VH CDR2 | EINSDSNTINYTPSLKD |
| NO: 383 | | |
| SEQ ID | VB22 VH CDR3 | GGLLRDV |
| NO: 384 | | |
| SEQ ID | VB11 VH CDR1 | DSAMH |
| NO: 385 | | |
| SEQ ID | VB11 VH CDR2 | VISSYNGNTNYNQKFKG |
| NO: 386 | | |
| SEQ ID | VB11 VH CDR3 | SRDAMDY |
| NO: 387 | | |
| SEQ ID | VB1 VH CDR1 | GYTFTDY |
| NO: 388 | | |
| SEQ ID | VB1 VH CDR2 | NTYTGT |
| NO: 389 | | |
| SEQ ID | VB1 VH CDR3 | SWRRGIRGIGFDY |
| NO: 390 | | |
| SEQ ID | VB12 VH CDR1 | GFTFRSY |
| NO: 391 | | |
| SEQ ID | VB12 VH CDR2 | SSGGSY |
| NO: 392 | | |
| SEQ ID | VB12 VH CDR3 | HGGNFFDY |
| NO: 393 | | |
| SEQ ID | VB8 VH CDR1 | GFTFSNF |
| NO: 394 | | |
| SEQ ID | VB8 VH CDR2 | SSGSST |
| NO: 395 | | |
| SEQ ID | VB8 VH CDR3 | RGEGAMDY |
| NO: 396 | | |
| SEQ ID | VB13.1 VH | GFTFRSY |
| NO: 397 | CDR1 | |
| SEQ ID | VB13.1 VH | SNGGDY |
| NO: 398 | CDR2 | |
| SEQ ID | VB13.1 VH | PSYYSDPWFFDV |
| NO: 399 | CDR3 | |
| SEQ ID | VB5.1 VH CDR1 | GYTFTDYNIH |
| NO: 400 | | |
| SEQ ID | VB5.1 VH CDR2 | NPYNGR |
| NO: 401 | | |
| SEQ ID | VB5.1 VH CDR3 | WDGSSYFDY |
| NO: 402 | | |
| SEQ ID | VB9 VH CDR1 | NTFMH |
| NO: 403 | | |
| SEQ ID | VB9 VH CDR2 | RIDPTNGYTKFAPKFQG |
| NO: 404 | | |
| SEQ ID | VB9 VH CDR3 | DYDAPWFAY |
| NO: 405 | | |
| SEQ ID | VB17 VH CDR1 | GYSITSGY |
| NO: 406 | | |
| SEQ ID | VB17 VH CDR2 | SYDGSN |
| NO: 407 | | |
| SEQ ID | VB17 VH CDR3 | PSPGTGYAVDY |
| NO: 408 | | |
| SEQ ID | VB3 VH CDR1 | GYTFSGS |
| NO: 409 | | |
| SEQ ID | VB3 VH CDR2 | YPGDGD |
| NO: 410 | | |
| SEQ ID | VB3 VH CDR3 | SGYFNYVPVFDY |
| NO: 411 | | |
| SEQ ID | VB7.1 VH CDR1 | GFTFSDY |
| NO: 412 | | |
| SEQ ID | VB7.1 VH CDR2 | SGGGSY |
| NO: 413 | | |
| SEQ ID | VB7.1 VH CDR3 | ERDIYYGNFNAMVY |
| NO: 414 | | |
| SEQ ID | VB5.3 VH CDR1 | GYAFSSS |
| NO: 415 | | |
| SEQ ID | VB5.3 VH CDR2 | YPGDGD |
| NO: 416 | | |
| SEQ ID | VB5.3 VH CDR3 | RGTGGWYFDV |
| NO: 417 | | |
| SEQ ID | VB7.2 VL CDR1 | KASQDVYTAVA |
| NO: 418 | | |
| SEQ ID | VB7.2 VL CDR2 | ASNRYT |
| NO: 419 | | |
| SEQ ID | VB7.2 VL CDR3 | QQHYTTPRT |
| NO: 420 | | |
| SEQ ID | VB4 VL CDR1 | LASEGISNFLA |
| NO: 421 | | |
| SEQ ID | VB4 VL CDR2 | YTSSLQD |
| NO: 422 | | |
| SEQ ID | VB4 VL CDR3 | QQGYKFPRT |
| NO: 423 | | |
| SEQ ID | VB13.2 VL | SASSSISSTNLH |
| NO: 424 | CDR1 | |
| SEQ ID | VB13.2 VL | GTSNLAS |
| NO: 425 | CDR2 | |
| SEQ ID | VB13.2 VL | QQWSRYPLT |
| NO: 426 | CDR3 | |
| SEQ ID | VB14 VL CDR1 | SESVDSYGNSF |
| NO: 427 | | |
| SEQ ID | VB14 VL CDR2 | RASNLES |
| NO: 428 | | |
| SEQ ID | VB14 VL CDR3 | QQSNEDPYT |
| NO: 429 | | |
| SEQ ID | VB22 VL CDR1 | RSSTGAVTTSNYAN |
| NO: 430 | | |
| SEQ ID | VB22 VL CDR2 | GTNNRAP |
| NO: 431 | | |
| SEQ ID | VB22 VL CDR3 | ALWYSNHWV |
| NO: 432 | | |
| SEQ ID | VB11 VL CDR1 | KASQDINSYLS |
| NO: 433 | | |
| SEQ ID | VB11 VL CDR2 | RANRLVD |
| NO: 434 | | |
| SEQ ID | VB11 VL CDR3 | LQYDEFPFT |
| NO: 435 | | |
| SEQ ID | VB1 VL CDR1 | SKSINKY |
| NO: 436 | | |
| SEQ ID | VB1 VL CDR2 | DGSTLQS |
| NO: 437 | | |
| SEQ ID | VB1 VL CDR3 | QQHNEYPPT |
| NO: 438 | | |
| SEQ ID | VB12 VL CDR1 | SSSVSY |
| NO: 439 | | |
| SEQ ID | VB12 VL CDR2 | DTSKLAS |
| NO: 440 | | |
| SEQ ID | VB12 VL CDR3 | QQWSSNPQYT |
| NO: 441 | | |
| SEQ ID | VB8 VL CDR1 | RASSSVNYIY |
| NO: 442 | | |
| SEQ ID | VB8 VL CDR2 | YTSNLAP |
| NO: 443 | | |
| SEQ ID | VB8 VL CDR3 | QQFTSSPFT |
| NO: 444 | | |
| SEQ ID | VB13.1 VL | GSSVSF |
| NO: 445 | CDR1 | |
| SEQ ID | VB13.1 VL | DTSKLAS |
| NO: 446 | CDR2 | |
| SEQ ID | VB13.1 VL | LQGSGFPLT |
| NO: 447 | CDR3 | |
| SEQ ID | VB5.1 VL CDR1 | SSSVSY |
| NO: 448 | | |
| SEQ ID | VB5.1 VL CDR2 | EISKLAS |
| NO: 449 | | |
| SEQ ID | VB5.1 VL CDR3 | QQWNYPLLT |
| NO: 450 | | |
| SEQ ID | VB9 VL CDR1 | ISSTDIDDDMN |
| NO: 451 | | |
| SEQ ID | VB9 VL CDR2 | EGNTLRP |
| NO: 452 | | |
| SEQ ID | VB9 VL CDR3 | LQSDNMPLT |
| NO: 453 | | |
| SEQ ID | VB17 VL CDR1 | SQSLVHSNGNTY |
| NO: 454 | | |
| SEQ ID | VB17 VL CDR2 | KVSNRFS |
| NO: 455 | | |
| SEQ ID | VB17 VL CDR3 | SQSTHVPFT |
| NO: 456 | | |
| SEQ ID | VB3 VL CDR1 | NSTVGY |
| NO: 457 | | |
| SEQ ID | VB3 VL CDR2 | TTSNLAS |
| NO: 458 | | |
| SEQ ID | VB3 VL CDR3 | HQWSFYPT |
| NO: 459 | | |
| SEQ ID | VB7.1 VL CDR1 | SKSVSTSGYSY |
| NO: 460 | | |
| SEQ ID | VB7.1 VL CDR2 | LASNLES |
| NO: 461 | | |
| SEQ ID | VB7.1 VL CDR3 | QHSRDLPWT |
| NO: 462 | | |
| SEQ ID | VB5.3 VL CDR1 | SESVDSSGNSF |
| NO: 463 | | |
| SEQ ID | VB5.3 VL CDR2 | RASNLES |
| NO: 464 | | |
| SEQ ID | VB5.3 VL CDR3 | QQSFDDPFT |
| NO: 465 | | |
| SEQ ID | VB 2 murine VH | EVQLQQSVADLVRPGASLKLSCTASGFNIKSAYMHWVIQRPDQGPECLGRI |
| NO: | | DPATGKTKYAPKFQAKATITADTSSNTAYLQLSSLTSEDTAIYYCTRSLNW |
| 466 | | DYGLDYWGQGTSVTVSS |
| SEQ ID | VB 2 murine VL | DIVLTQSPASLAVSLGQRATISCRASKSVSILGTHLIHWYQQKPGQPPKLLIY |
| NO: | | AASNLESGVPARFSGSGSETVFTLNIHPVEEEDAATYFCQQSIEDPWTFGGG |
| 467 | | TKLGIK |
| SEQ ID | VB 7.2 murine | QVHLQQSGAELARPGASVKMSCKASGYIFTDYTMHWVKQRPGQCLEWIG |
| NO: | VH | HINPSSGYSTYNQKFKDKATLTADKSSSTAYMQLSSLTSEDSAVYYCARSL |
| 468 | | QLGRDYWGQGTTLTVSS |
| SEQ ID | VB 7.2 murine | DIVMTQSHKFMSTSVGDRVSITCKASQDVYTAVAWYQQKPGQSPKLLIYS |
| NO: | VL | ASNRYTGVPDRFTGSGSGTDFTFTISSVQAEDLAVYYCQQHYTTPRTFGCG |
| 469 | | TKLEIK |
| SEQ ID | VB 4 murine VH | QVQLQQSGAELAKPGTSVKLSCKASGYTFTSYYIYWVKQRPGQCLEWLGY |
| NO: | | IYPGNGGTYYSEKFKGKATFTADTSSNTAYMLLGSLTPEDSAYYFCARGSG |
| 470 | | DRYNSLAYWGQGTLVTVSS |
| SEQ ID | VB 4 murine VL | DIQMTQSPHSLSASLGETVSIECLASEGISNFLAWYQQKPGKSPQLLIYYTSS |
| NO: | | LQDGVPSRFSGSGSGTQYSLKISNMQPEDEGVYYCQQGYKFPRTFGCGTKL |
| 471 | | ELK |
| SEQ ID | VB 13.2 murine | EVQLQQSGPELVKPGASVKMSCNASGYTFTDYYIHWLKQRHGKCLEWIGI |
| NO: | VH | VNTNNGDTNYNQRFKGKASLTVDKSSSTAYMELNSLTSEDSAVFYCARAL |
| 472 | | YTGSYWFAYWGQGTLVTVSA |
| SEQ ID | VB 13.2 murine | EIVLTQSPAVMAASPGEKVTITCSASSSISSTNLHWYQQKSETSPKPWIYGTS |
| NO: | VL | NLASGVPVRFSGSGSGTSYSLTISSIEAEDAATYYCQQWSRYPLTFGCGTKL |
| 473 | | EII |
| SEQ ID | VB 14 murine VH | EVQLQQSGAELVKPGASVKLSCTASGFKTEDTYMYWVKQRPEQCLEWIG |
| NO: | | RIDPANGNTKYDPKFQDKATITADSSSNTAYLQLSSLPSEDTAVYYCARGS |
| 474 | | YYYAMDYWGQGTSVTVSS |
| SEQ ID | VB 14 murine VL | DIVLTQSPASLAVSLGQRATISCRASESVDSYGNSFMHWYQQKPGQPPKLLI |
| NO: | | YRASNLESGIPARFSGSGSRTDFTLTINPVEADDVATYYCQQSNEDPYTFGC |
| 475 | | GTKLEIK |
| SEQ ID | VB22 murine VH | EVKLLESGGGLVQPGGSLKLSCAASGFDFSRYWMNWVRQAPGKCLEWIG |
| NO: | | EINSDSNTINYTPSLKDKFIISRDNAKNTLYLQMNKVRSEDTALYYCARGGL |
| 476 | | LRDVWGAGTTVTVSS |
| SEQ ID | VB11 murine VH | QVQLQQSGPEVVRPGVSVKISCKGSGYRFTDSAMHWVKQSHAKCLEWIG |
| NO: | | VISSYNGNTNYNQKFKGKATMTVDKSSSTAYMELARMTSEDSAIYYCARS |
| 477 | | RDAMDYWGQGTSVTVSS |
| SEQ ID | VB11 murine VL | DIKMTQSPSSMYASLGERVTITCKASQDINSYLSWFQQKAGKSPKTLIYRA |
| NO: | | NRLVDGVPSRFSGSGSGQDYSLTISSLEYEDMGIYYCLQYDEFPFTFGCGTR |
| 478 | | LEIK |
| SEQ ID | VB1 murine VH | QLQLVQSGPELREPGESVKISCKASGYTFTDYIVHWVKQAPGKCLKWMG |
| NO: | | WINTYTGTPTYADDFEGRFVFSLEASASTANLQISNLKNEDTATYFCARSW |
| 479 | | RRGIRGIGFDWGQGVMVTVSS |
| SEQ ID | VB1 murine VL | DVQMTQSPYNLAASPGESVSINCKASKSINKYLAWYQQKPGKPNKLLIYD |
| NO: | | GSTLQSGIPSRFSGSGSGTDFTLTIRGLEPEDFGLYYCQQHNEYPPTFGCGTK |
| 480 | | LELK |
| SEQ ID | VB12 murine VH | EVQLVESGGDLVKPGGSLKLSCAVSGFTFRSYGMSWVRQTPDKCLEWVA |
| NO: | | LISSGGSYTYYTDSVKGRFTISRDNAKNTLYLQMSSLKSEDTAIYYCSRHGG |
| 481 | | NFFDYWGQGTTLTVSS |
| SEQ ID | VB12 murine VL | QIVLTQSPSIMSASPGEKVTMTCSVSSSVSYMEIWYQQKSGTSPKRWIYDTS |
| NO: | | KLASGVPARFSGSGSGTSYSLTISSMEAEDAATYYCQQWSSNPQYTFGCGT |
| 482 | | KLEIK |
| SEQ ID | VB8 murine VH | EVQLVESGGGLVQPGGSRKLSCAASGFTFSNFGMHWVRQAPGKCLEWVA |
| NO: | | YISSGSSTIYYADTLKGRFTISRDNPKNTLFLQMTSLRSEDTAMYYCARRGE |
| 483 | | GAMDYWGQGTSVTVSS |
| SEQ ID | VB8 murine VL | ENVLTQSPAIMSASLGEKVTMSCRASSSVNYIYQYQQKSDASPKLWIYYTS |
| NO: | | NLAPGVPTRFSGSGSGNSYSLTISSMEGEDAATYYCQQFTSSPFTFGCGTKL |
| 484 | | EIK |
| SEQ ID | VB13.1 murine | DVKLVESGEGLVKPGGSLKLSCAASGFTFRSYAMSWVRQTPEKCLEWVA |
| NO: | VH | HISNGGDYIYYADTVKGRFTISRDNARNTLYLQMSSLKSEDTAMYYCTRPS |
| 485 | | YYSDPWFFDVWGTGTTVTVSS |
| SEQ ID | VB13.1 murine | ENVLTQSPAIMSASPGEKVTMTCSAGSSVSFMEIWYQQKSSTSPKLWIYDT |
| NO: | VL | SKLASGVPGRFSGSGSGNSFSLTISMEAEDVAIYYCLQGSGFPLTFGCGTKL |
| 486 | | EIK |
| SEQ ID | VB5.1 murine VH | EVQLQQSGPVLVKPGASVRMSCKASGYTFTDYNIHWVKQSHGRCLEWVG |
| NO: | | YINPYNGRTGYNQKFKAKATLTVDKSSSTAYMDLRSLTSEDSAVYYCARW |
| 487 | | DGSSYFDYWGQGTTLTVSS |
| SEQ ID | VB5.1 murine VL | EIVLTQSPAITAASLGQKVTITCSASSSVSYMHWYQQKSGTSPKPWIYEISK |
| NO: | | LASGVPARFSGSGSGTSYSLTISSMEAEDAAIYYCQQWNYPLLTFGCGTKL |
| 488 | | ELK |
| SEQ ID | VB9 murine VH | EVQLQQSVAELVRPGASVKLSCTASGFNIKNTFMHWVKQRPEQCLEWIGRI |
| NO: | | DPTNGYTKFAPKFQGKATLTAVTSSNTVYLQLSSLTSEDTAIYYCAHDYDA |
| 489 | | PWFAYWGQGTLVIVSA |
| SEQ ID | VB9 murine VL | ETTVTQSPASLSVATGEKVTIRCISSTDIDDDMNWYQQKSGEPPKLLISEGN |
| NO: | | TLRPGVPSRFSSSGYGTDFVFTIENMLSEDVADYYCLQSDNMPLTFGCGTK |
| 490 | | LELK |
| SEQ ID | VB17 murine VH | NVQLQESGPGLVKPSQSLSLTCSVAGYSITSGYFWNWIRQFPGNCLEWMG |
| NO: | | YISYDGSNNYNPSLKNRISITRDTSKNQFFLKLNSVTTEDTATYYCASPSPGT |
| 491 | | GYAVDYWGQGTSVTVSS |
| SEQ ID | VB17 murine VL | NVVMTQTPLSLPVSLGDQASISCRSSQSLVHSNGNTYLHWYLQKPGQSPKF |
| NO: | | LIYKVSNRFSGVPDRFSGGGSGTEFTLKISRVEAEDLGVYFCSQSTHVPFTF |
| 492 | | GCGTKLEIK |
| SEQ ID | VB3 murine VH | QIQLQQSGPEVVKPGASVQISCKASGYTFSGSWMNWVKQRPGKCLEWIGR |
| NO: | | IYPGDGDTDYSGKFKGRATLTADKSSSTAYMRLSSLTSEDSAVYFCARSGY |
| 493 | | FNYVPVFDYWGQGTTLSVSS |
| SEQ ID | VB3 murine VL | QIVLTQSPAIMSASLGEEIALTCSANSTVGYIEIWYQQKSGTSPKLLIYTTSN |
| NO: | | LASGVPSRFSGSGSGTFYSLTISSVEAEDAADYFCHQWSFYPTFGCGTKLEI |
| 494 | | K |
| SEQ ID | VB7.1 murine VH | EVQLVESGGGLVKPGGSLKLSCAASGFTFSDYYMYWVRQTPEKCLEWVA |
| NO: | | TISGGGSYTYSPDSVKGRFTISRDNAKNNLYLQMSSLRSEDTAMYFCARER |
| 495 | | DIYYGNFNAMVYWGRGTSVTVSS |
| SEQ ID | VB7.1 murine VL | DIVLTQSPVSLTVSLGQRATISCRASKSVSTSGYSYMHWYQQKPGQPPKLLI |
| NO: | | YLASNLESGVPARFSGSGSGTDFTLNIHPVEEEDAATYYCQHSRDLPWTFG |
| 496 | | CGTKLEIK |
| SEQ ID | VB5.3 murine VH | QVQLQQSGPELVKPGASVKISCKASGYAFSSSWMNWVKQRPGQCLEWIGR |
| NO: | | IYPGDGDTKYNGKFKGKATLTADKSSSTAYMHLSSLTSVDSAVYFCARRG |
| 497 | | TGGWYFDVWGAGTTVTVSS |
| SEQ ID | VB5.3 murine VL | DIVLTQSPASLAVSLGQRATISCRASESVDSSGNSFMHWYQQKPGQPPQLLI |
| NO: | | YRASNLESGIPARFSGSGSRTDFTLTINPVEADDVATFYCQQSFDDPFTFGC |
| 498 | | GTKLEIK |
| SEQ ID | anti-VB 2 murine | EVQLQQSVADLVRPGASLKLSCTASGFNIKSAYMHWVIQRPDQGPECLGRI |
| NO: | VH | DPATGKTKYAPKFQAKATITADTSSNTAYLQLSSLTSEDTAIYYCTRSLNW |
| 499 | | DYGLDYWGQGTSVTVSS |
| SEQ ID | anti-VB 2 murine | DIVLTQSPASLAVSLGQRATISCRASKSVSILGTHLIHWYQQKPGQPPKLLIY |
| NO: | VL | AASNLESGVPARFSGSGSETVFTLNIHPVEEEDAATYFCQQSIEDPWTFGGG |
| 500 | | TKLGIK |
| SEQ ID | anti-VB 7.2 | QVHLQQSGAELARPGASVKMSCKASGYIFTDYTMHWVKQRPGQGLEWIG |
| NO: | murine VH | HINPSSGYSTYNQKFKDKATLTADKSSSTAYMQLSSLTSEDSAVYYCARSL |
| 501 | | QLGRDYWGQGTTLTVSS |
| SEQ ID | anti-VB 7.2 | DIVMTQSHKFMSTSVGDRVSITCKASQDVYTAVAWYQQKPGQSPKLLIYS |
| NO: | murine VL | ASNRYTGVPDRFTGSGSGTDFTFTISSVQAEDLAVYYCQQHYTTPRTFGGG |
| 502 | | TKLEIK |
| SEQ ID | anti-VB 4 murine | QVQLQQSGAELAKPGTSVKLSCKASGYTFTSYYIYWVKQRPGQGLEWLG |
| NO: | VH | YIYPGNGGTYYSEKFKGKATFTADTSSNTAYMLLGSLTPEDSAYYFCARGS |
| 503 | | GDRYNSLAYWGQGTLVTVSS |
| SEQ ID | anti-VB 4 murine | DIQMTQSPHSLSASLGETVSIECLASEGISNFLAWYQQKPGKSPQLLIYYTSS |
| NO: | VL | LQDGVPSRFSGSGSGTQYSLKISNMQPEDEGVYYCQQGYKFPRTFGGGTKL |
| 504 | | ELK |
| SEQ ID | anti-VB 4 murine | DIQMTQSPSSLSASVGDTVTITCLASEGISNFLAWYQQKPGKAPKLLIYYTS |
| NO: | VL1 | SLQDGVPSRFSGSGSGTDFSLTISNLQPEDFGTYYCQQGYKFPRTFGQGTKV |
| 505 | | EIK |
| SEQ ID | anti-VB 13.2 | EVQLQQSGPELVKPGASVKMSCNASGYTFTDYYIHWLKQRHGKGLEWIGI |
| NO: | murine VH | VNTNNGDTNYNQRFKGKASLTVDKSSSTAYMELNSLTSEDSAVFYCARAL |
| 506 | | YTGSYWFAYWGQGTLVTVSA |
| SEQ ID | anti-VB 13.2 | EIVLTQSPAVMAASPGEKVTITCSASSSISSTNLHWYQQKSETSPKPWIYGTS |
| NO: | murine VL | NLASGVPVRFSGSGSGTSYSLTISSIEAEDAATYYCQQWSRYPLTFGSGTKL |
| 507 | | EII |
| SEQ ID | anti-VB 14 | EVQLQQSGAELVKPGASVKLSCTASGFKTEDTYMYWVKQRPEQGLEWIG |
| NO: | murine VH | RIDPANGNTKYDPKFQDKATITADSSSNTAYLQLSSLPSEDTAVYYCARGS |
| 508 | | YYYAMDYWGQGTSVTVSS |
| SEQ ID | anti-VB 14 | DIVLTQSPASLAVSLGQRATISCRASESVDSYGNSFMHWYQQKPGQPPKLLI |
| NO: | murine VL | YRASNLESGIPARFSGSGSRTDFTLTINPVEADDVATYYCQQSNEDPYTFGG |
| 509 | | GTKLEIK |
| SEQ ID | anti-VB22 murine | EVKLLESGGGLVQPGGSLKLSCAASGFDFSRYWMNWVRQAPGKGLEWIG |
| NO: | VH | EINSDSNTINYTPSLKDKFIISRDNAKNTLYLQMNKVRSEDTALYYCARGGL |
| 510 | | LRDVWGAGTTVTVSS |
| SEQ ID | anti-VB22 murine | QAVVTQESALTTSPGETVTLTCRSSTGAVTTSNYANWVQEKPDHLFTGLIG |
| NO: | VL | GTNNRAPGVPARESGSLIGDRAALTITGAQTEDEAIYFCALWYSNHWVFG |
| 511 | | GGTKLTVL |
| SEQ ID | anti-VB11 murine | QVQLQQSGPEVVRPGVSVKISCKGSGYRFTDSAMHWVKQSHAKSLEWIGV |
| NO: | VH | ISSYNGNTNYNQKFKGKATMTVDKSSSTAYMELARMTSEDSAIYYCARSR |
| 512 | | DAMDYWGQGTSVTVSS |
| SEQ ID | anti-VB11 murine | DIKMTQSPSSMYASLGERVTITCKASQDINSYLSWFQQKAGKSPKTLIYRA |
| NO: | VL | NRLVDGVPSRFSGSGSGQDYSLTISSLEYEDMGIYYCLQYDEFPFTFGGGTR |
| 513 | | LEIK |
| SEQ ID | anti-VB1 murine | QLQLVQSGPELREPGESVKISCKASGYTFTDYIVHWVKQAPGKGLKWMG |
| NO: | VH | WINTYTGTPTYADDFEGRFVFSLEASASTANLQISNLKNEDTATYFCARSW |
| 514 | | RRGIRGIGFDWGQGVMVTVSS |
| SEQ ID | anti-VB1 murine | DVQMTQSPYNLAASPGESVSINCKASKSINKYLAWYQQKPGKPNKLLIYD |
| NO: | VL | GSTLQSGIPSRFSGSGSGTDFTLTIRGLEPEDFGLYYCQQHNEYPPTFGAGTK |
| 515 | | LELK |
| SEQ ID | anti-VB12 murine | EVQLVESGGDLVKPGGSLKLSCAVSGFTFRSYGMSWVRQTPDKRLEWVA |
| NO: | VH | LISSGGSYTYYTDSVKGRFTISRDNAKNTLYLQMSSLKSEDTAIYYCSRHGG |
| 516 | | NFFDYWGQGTTLTVSS |
| SEQ ID | anti-VB12 murine | QIVLTQSPSIMSASPGEKVTMTCSVSSSVSYMEIWYQQKSGTSPKRWIYDTS |
| NO: | VL | KLASGVPARFSGSGSGTSYSLTISSMEAEDAATYYCQQWSSNPQYTFGGGT |
| 517 | | KLEIK |
| SEQ ID | anti-VB8 murine | EVQLVESGGGLVQPGGSRKLSCAASGFTFSNFGMHWVRQAPGKGLEWVA |
| NO: | VH | YISSGSSTIYYADTLKGRFTISRDNPKNTLFLQMTSLRSEDTAMYYCARRGE |
| 518 | | GAMDYWGQGTSVTVSS |
| SEQ ID | anti-VB8 murine | ENVLTQSPAIMSASLGEKVTMSCRASSSVNYIYQYQQKSDASPKLWIYYTS |
| NO: | VL | NLAPGVPTRFSGSGSGNSYSLTISSMEGEDAATYYCQQFTSSPFTFGSGTKL |
| 519 | | EIK |
| SEQ ID | anti-VB13.1 | DVKLVESGEGLVKPGGSLKLSCAASGFTFRSYAMSWVRQTPEKRLEWVA |
| NO: | murine VH | HISNGGDYIYYADTVKGRFTISRDNARNTLYLQMSSLKSEDTAMYYCTRPS |
| 520 | | YYSDPWFFDVWGTGTTVTVSS |
| SEQ ID | anti-VB13.1 | ENVLTQSPAIMSASPGEKVTMTCSAGSSVSFMEIWYQQKSSTSPKLWIYDT |
| NO: | murine VL | SKLASGVPGRFSGSGSGNSFSLTISMEAEDVAIYYCLQGSGFPLTFGSGTKL |
| 521 | | EIK |
| SEQ ID | anti-VB5.1 | EVQLQQSGPVLVKPGASVRMSCKASGYTFTDYNIHWVKQSHGRSLEWVG |
| NO: | murine VH | YINPYNGRTGYNQKFKAKATLTVDKSSSTAYMDLRSLTSEDSAVYYCARW |
| 522 | | DGSSYFDYWGQGTTLTVSS |
| SEQ ID | anti-VB5.1 | EIVLTQSPAITAASLGQKVTITCSASSSVSYMHWYQQKSGTSPKPWIYEISK |
| NO: | murine VL | LASGVPARFSGSGSGTSYSLTISSMEAEDAAIYYCQQWNYPLLTFGAGTKL |
| 523 | | ELK |
| SEQ ID | anti-VB9 murine | EVQLQQSVAELVRPGASVKLSCTASGFNIKNTFMHWVKQRPEQGLEWIGR |
| NO: | VH | IDPTNGYTKFAPKFQGKATLTAVTSSNTVYLQLSSLTSEDTAIYYCAHDYD |
| 524 | | APWFAYWGQGTLVIVSA |
| SEQ ID | anti-VB9 murine | ETTVTQSPASLSVATGEKVTIRCISSTDIDDDMNWYQQKSGEPPKLLISEGN |
| NO: | VL | TLRPGVPSRFSSSGYGTDFVFTIENMLSEDVADYYCLQSDNMPLTFGAGTK |
| 525 | | LELK |
| SEQ ID | anti-VB17 murine | NVQLQESGPGLVKPSQSLSLTCSVAGYSITSGYFWNWIRQFPGNKLEWMG |
| NO: | VH | YISYDGSNNYNPSLKNRISITRDTSKNQFFLKLNSVTTEDTATYYCASPSPGT |
| 526 | | GYAVDYWGQGTSVTVSS |
| SEQ ID | anti-VB17 murine | NVVMTQTPLSLPVSLGDQASISCRSSQSLVHSNGNTYLHWYLQKPGQSPKF |
| NO: | VL | LIYKVSNRFSGVPDRFSGGGSGTEFTLKISRVEAEDLGVYFCSQSTHVPFTF |
| 527 | | GSGTKLEIK |
| SEQ ID | anti-VB3 murine | QIQLQQSGPEVVKPGASVQISCKASGYTFSGSWMNWVKQRPGKGLEWIGR |
| NO: | VH | IYPGDGDTDYSGKFKGRATLTADKSSSTAYMRLSSLTSEDSAVYFCARSGY |
| 528 | | FNYVPVFDYWGQGTTLSVSS |
| SEQ ID | anti-VB3 murine | QIVLTQSPAIMSASLGEEIALTCSANSTVGYIEIWYQQKSGTSPKLLIYTTSN |
| NO: | VL | LASGVPSRFSGSGSGTFYSLTISSVEAEDAADYFCHQWSFYPTFGGGTKLEI |
| 529 | | K |
| SEQ ID | anti-VB7.1 | EVQLVESGGGLVKPGGSLKLSCAASGFTFSDYYMYWVRQTPEKRLEWVA |
| NO: | murine VH | TISGGGSYTYSPDSVKGRFTISRDNAKNNLYLQMSSLRSEDTAMYFCARER |
| 530 | | DIYYGNFNAMVYWGRGTSVTVSS |
| SEQ ID | anti-VB7.1 | DIVLTQSPVSLTVSLGQRATISCRASKSVSTSGYSYMHWYQQKPGQPPKLLI |
| NO: | murine VL | YLASNLESGVPARFSGSGSGTDFTLNIHPVEEEDAATYYCQHSRDLPWTFG |
| 531 | | GGTKLEIK |
| SEQ ID | anti-VB5.3 | QVQLQQSGPELVKPGASVKISCKASGYAFSSSWMNWVKQRPGQGLEWIG |
| NO: | murine VH | RIYPGDGDTKYNGKFKGKATLTADKSSSTAYMHLSSLTSVDSAVYFCARR |
| 532 | | GTGGWYFDVWGAGTTVTVSS |
| SEQ ID | anti-VB5.3 | DIVLTQSPASLAVSLGQRATISCRASESVDSSGNSFMHWYQQKPGQPPQLLI |
| NO: | murine VL | YRASNLESGIPARFSGSGSRTDFTLTINPVEADDVATFYCQQSFDDPFTFGS |
| 533 | | GTKLEIK |
| SEQ ID | VB20 humanized | EVQLVQSGAEVKKPGASVKLSCTASGFNIKDYYIHWVRQAPGQCLEWMG |
| NO: | VH 1 | RIDPEDGETNYAPKFQGRVTISADTSSNTAFLQLSSLTSEDTAVYYCARSDL |
| 534 | | ILFAYWGQGTLVTVSS |
| SEQ ID | VB20 humanized | EVQLVQSGAEVKKPGATVKISCKVSGFNIKDYYMHWVQQAPGKCLEWM |
| NO: | VH 2 | GLVDPEDGETIYAEKFQGRVTITADTSTDTAYMELSSLRSEDTAVYYCATS |
| 535 | | DLILFAYWGQGTLVTVSS |
| SEQ ID | VB20 humanized | QVQLVQSGAEVKKPGASVKVSCKASGFNIKDYYMHWVRQAPGQCLEWM |
| NO: | VH 3 | GRIDPEDGETNYAQKFQGRVTMTRDTSISTAYMELSRLRSDDTAVYYCAR |
| 536 | | SDLILFAYWGQGTLVTVSS |
| SEQ ID | VB18 humanized | EVQLLESGGGLVQPGGSLRLSCAASGFDFSRYWMTWVRQAPGKCLEWVS |
| NO: | VH 1 | NINSDSSTINYADSVKGRFTISRDNAKNSLYLQMSSLRAEDTALYYCASPY |
| 537 | | YYGSSSWFAYWGQGTLVTVSS |
| SEQ ID | VB18 humanized | EVQLVESGGGLVQPGGSLRLSCAASGFDFSRYSMNWVRQAPGKCLEWVS |
| NO: | VH 2 | YINSDSSTIYYADSVKGRFTISRDNAKNSLYLQMNSLRAEDTAVYYCARPY |
| 538 | | YYGSSSWFAYWGQGTLVTVSS |
| SEQ ID | VB18 humanized | EVQLVESGGGLVKPGGSLRLSCAASGFDFSRYSMNWVRQAPGKCLEWVSS |
| NO: | VH 3 | INSDSSTIYYADSVKGRFTISRDNAKNSLYLQMNSLRAEDTAVYYCARPYY |
| 539 | | YGSSSWFAYWGQGTLVTVSS |
| SEQ ID | VB13.6 | QVQLVQSGPELKKPGASVKISCKASGYSFTGNYMHWVRQAPGKCLEWMG |
| NO: | humanized VH 1 | WINPNTGGTTYAQKLKGRVTLTVDKSSSTAYMELSSLTSEDTAVYYCARS |
| 540 | | EYYGIYYYAMDYWGQGTTVTVSS |
| SEQ ID | VB13.6 | QVQLVQSGAEVKKPGASVKVSCKASGYSFTGNYMHWVRQAPGQCLEWM |
| NO: | humanized VH 2 | GWINPNTGGTNYAQKFQGRVTMTRDTSISTAYMELSRLRSDDTAVYYCAR |
| 541 | | SEYYGIYYYAMDYWGQGTTVTVSS |
| SEQ ID | VB16 humanized | QVQLVQSGTELKKPGASVKVSCKASGYTFTSYYMHWVRQAPGQCLEWM |
| NO: | VH 1 | GWINPSNGGTYYAQKFQGRVTLTRDTSSSTAYMELSSLTSEDTAVYYCAR |
| 542 | | DSYYFDYWGQGTLVTVSS |
| SEQ ID | VB16 humanized | QVQLVQSGAEVKKPGASVKVSCKASGYTFTSYYMHWVRQAPGQCLEWM |
| NO: | VH 2 | GIINPSNGGTSYAQKFQGRVTMTRDTSTSTVYMELSSLRSEDTAVYYCARD |
| 543 | | SYYFDYWGQGTLVTVSS |
| SEQ ID | VB16 humanized | QVQLVQSGAEVKKPGASVKVSCKASGYTFTSYYMHWVRQAPGQCLEWM |
| NO: | VH 3 | GWINPSNGGTNYAQKFQGRVTMTRDTSISTAYMELSRLRSDDTAVYYCAR |
| 544 | | DSYYFDYWGQGTLVTVSS |
| SEQ ID | VB20 humanized | DVVMTQTPLSLPVTLGQPASISCRSSQSIVHSNRYTYLEWYLQKPGQSPQLL |
| NO: | VL 1 | IYGVSNRFSGVPDRFSGSGSGTDFTLKISRVEAEDVGVYYCFQGTHVPFMF |
| 545 | | GCGTKLEIK |
| SEQ ID | VB20 humanized | DIVMTQTPLSLSVTPGQPASISCRSSQSIVHSNRYTYLEWYLQKPGQSPQLLI |
| NO: | VL 2 | YGVSNRFSGVPDRFSGSGSGTDFTLKISRVEAEDVGVYYCFQGTHVPFMFG |
| 546 | | CGTKLEIK |
| SEQ ID | VB20 humanized | DVVMTQSPLSLPVTLGQPASISCRSSQSIVHSNRYTYLEWFQQRPGQSPRRL |
| NO: | VL 3 | IYGVSNRFSGVPDRFSGSGSGTDFTLKISRVEAEDVGVYYCFQGTHVPFMF |
| 547 | | GCGTKLEIK |
| SEQ ID | VB18 humanized | DIVLTQSPATLSVSPGDRVTLSCRASQSISNFLHWYQQKPGQSPRLLIKYAS |
| NO: | VL 1 | QSISGIPSRFSGSGSGTDFTLTINSLEPEDFAVYFCQQSNSWPLTFGCGTKLEI |
| 548 | | K |
| SEQ ID | VB18 humanized | EIVLTQSPATLSLSPGERATLSCRASQSISNFLHWYQQKPGQAPRLLIYYAS |
| NO: | VL 2 | QSISGIPARFSGSGSGTDFTLTISSLEPEDFAVYYCQQSNSWPLTFGCGTKLEI |
| 549 | | K |
| SEQ ID | VB18 humanized | EIVLTQSPDFQSVTPKEKVTITCRASQSISNFLHWYQQKPDQSPKLLIKYASQ |
| NO: | VL 3 | SISGVPSRFSGSGSGTDFTLTINSLEAEDAATYYCQQSNSWPLTFGCGTKLEI |
| 550 | | K |
| SEQ ID | VB13.6 | DIQMTQSPSSLSASVGDTVTITCRTSENIYSNLVWYQQKPGKAPQLLIYAAT |
| NO: | humanized VL 1 | NLAEGVPSRFSGSGSGTDFSLTINSLQPEDFGTYYCQHFYGTPRTFGCGTKV |
| 551 | | EIK |
| SEQ ID | VB13.6 | DIQMTQSPSSLSASVGDRVTITCRTSENIYSNLVWYQQKPGKAPKLLIYAAT |
| NO: | humanized VL 2 | NLAEGVPSRFSGSGSGTDFTLTISSLQPEDFATYYCQHFYGTPRTFGCGTKL |
| 552 | | EIK |
| SEQ ID | VB16 humanized | DIQMTQSPSSLSASVGDRVTLTCRASQDISGYLSWYQQKPGKAPKLLIYSTS |
| NO: | VL-1 1 | TLDSGVPSRFSGSGSGTDFSLTISSLQSEDFATYYCLQYTTSPYTFGCGTKLE |
| 553 | | IK |
| SEQ ID | VB16 humanized | DIQMTQSPSSLSASVGDRVTITCRASQDISGYLSWFQQKPGKAPKSLIYSTST |
| NO: | VL-12 | LDSGVPSRFSGSGSGTDFTLTISSLQPEDFATYYCLQYTTSPYTFGCGTKLEI |
| 554 | | K |
| SEQ ID | VB16 humanized | DIQMTQSPSSLSASVGDRVTITCRASQDISGYLSWYQQKPGKVPKLLIYSTS |
| NO: | VL-13 | TLDSGVPSRFSGSGSGTDFTLTISSLQPEDVATYYCLQYTTSPYTFGCGTKL |
| 555 | | EIK |
| SEQ ID | VB16 humanized | DIQMTQSPSTLSASVGDRVTITCKASDHINNWLAWYQQKPGKAPRLLIYGA |
| NO: | VL-3 1 | TSLETGVPSRFSGSGSGTDFTLTITSLQPEDVATYYCQQYWSTPIFTFGCGTK |
| 556 | | LEIK |
| SEQ ID | VB16 humanized | DIQMTQSPSSVSASVGDRVTITCKASDHINNWLAWYQQKPGKAPKLLIYG |
| NO: | VL-3 2 | ATSLETGVPSRFSGSGSGTDFTLTISSLQPEDFATYYCQQYWSTPIFTFGCGT |
| 557 | | KLEIK |
| SEQ ID | VB16 humanized | DIQMTQSPSSLSASVGDRVTITCKASDHINNWLAWYQQKPGKAPKSLIYGA |
| NO: | VL-3 3 | TSLETGVPSRFSGSGSGTDFTLTISSLQPEDFATYYCQQYWSTPIFTFGCGTK |
| 558 | | LEIK |
| SEQ ID | anti-VB20 | EVQLVQSGAEVKKPGASVKLSCTASGFNIKDYYIHWVRQAPGQGLEWMG |
| NO: | humanized VH 1 | RIDPEDGETNYAPKFQGRVTISADTSSNTAFLQLSSLTSEDTAVYYCARSDL |
| 559 | | ILFAYWGQGTLVTVSS |
| SEQ ID | anti-VB20 | EVQLVQSGAEVKKPGATVKISCKVSGFNIKDYYMHWVQQAPGKGLEWM |
| NO: | humanized VH 2 | GLVDPEDGETIYAEKFQGRVTITADTSTDTAYMELSSLRSEDTAVYYCATS |
| 560 | | DLILFAYWGQGTLVTVSS |
| SEQ ID | anti-VB20 | QVQLVQSGAEVKKPGASVKVSCKASGFNIKDYYMHWVRQAPGQGLEWM |
| NO: | humanized VH 3 | GRIDPEDGETNYAQKFQGRVTMTRDTSISTAYMELSRLRSDDTAVYYCAR |
| 561 | | SDLILFAYWGQGTLVTVSS |
| SEQ ID | anti-VB18 | EVQLLESGGGLVQPGGSLRLSCAASGFDFSRYWMTWVRQAPGKGLEWVS |
| NO: | humanized VH 1 | NINSDSSTINYADSVKGRFTISRDNAKNSLYLQMSSLRAEDTALYYCASPY |
| 562 | | YYGSSSWFAYWGQGTLVTVSS |
| SEQ ID | anti-VB18 | EVQLVESGGGLVQPGGSLRLSCAASGFDFSRYSMNWVRQAPGKGLEWVS |
| NO: | humanized VH 2 | YINSDSSTIYYADSVKGRFTISRDNAKNSLYLQMNSLRAEDTAVYYCARPY |
| 563 | | YYGSSSWFAYWGQGTLVTVSS |
| SEQ ID | anti-VB18 | EVQLVESGGGLVKPGGSLRLSCAASGFDFSRYSMNWVRQAPGKGLEWVS |
| NO: | humanized VH 3 | SINSDSSTIYYADSVKGRFTISRDNAKNSLYLQMNSLRAEDTAVYYCARPY |
| 564 | | YYGSSSWFAYWGQGTLVTVSS |
| SEQ ID | anti-VB13.6 | QVQLVQSGPELKKPGASVKISCKASGYSFTGNYMHWVRQAPGKGLEWMG |
| NO: | humanized VH 1 | WINPNTGGTTYAQKLKGRVTLTVDKSSSTAYMELSSLTSEDTAVYYCARS |
| 565 | | EYYGIYYYAMDYWGQGTTVTVSS |
| SEQ ID | anti-VB13.6 | QVQLVQSGAEVKKPGASVKVSCKASGYSFTGNYMHWVRQAPGQGLEWM |
| NO: | humanized VH 2 | GWINPNTGGTNYAQKFQGRVTMTRDTSISTAYMELSRLRSDDTAVYYCAR |
| 566 | | SEYYGIYYYAMDYWGQGTTVTVSS |
| SEQ ID | anti-VB16 | QVQLVQSGTELKKPGASVKVSCKASGYTFTSYYMHWVRQAPGQGLEWM |
| NO: | humanized VH 1 | GWINPSNGGTYYAQKFQGRVTLTRDTSSSTAYMELSSLTSEDTAVYYCAR |
| 567 | | DSYYFDYWGQGTLVTVSS |
| SEQ ID | anti-VB16 | QVQLVQSGAEVKKPGASVKVSCKASGYTFTSYYMHWVRQAPGQGLEWM |
| NO: | humanized VH 2 | GIINPSNGGTSYAQKFQGRVTMTRDTSTSTVYMELSSLRSEDTAVYYCARD |
| 568 | | SYYFDYWGQGTLVTVSS |
| SEQ ID | anti-VB16 | QVQLVQSGAEVKKPGASVKVSCKASGYTFTSYYMHWVRQAPGQGLEWM |
| NO: | humanized VH 3 | GWINPSNGGTNYAQKFQGRVTMTRDTSISTAYMELSRLRSDDTAVYYCAR |
| 569 | | DSYYFDYWGQGTLVTVSS |
| SEQ ID | anti-VB20 | DVVMTQTPLSLPVTLGQPASISCRSSQSIVHSNRYTYLEWYLQKPGQSPQLL |
| NO: | humanized VL 1 | IYGVSNRFSGVPDRFSGSGSGTDFTLKISRVEAEDVGVYYCFQGTHVPFMF |
| 570 | | GQGTKLEIK |
| SEQ ID | anti-VB20 | DIVMTQTPLSLSVTPGQPASISCRSSQSIVHSNRYTYLEWYLQKPGQSPQLLI |
| NO: | humanized VL 2 | YGVSNRFSGVPDRFSGSGSGTDFTLKISRVEAEDVGVYYCFQGTHVPFMFG |
| 571 | | QGTKLEIK |
| SEQ ID | anti-VB20 | DVVMTQSPLSLPVTLGQPASISCRSSQSIVHSNRYTYLEWFQQRPGQSPRRL |
| NO: | humanized VL 3 | IYGVSNRFSGVPDRFSGSGSGTDFTLKISRVEAEDVGVYYCFQGTHVPFMF |
| 572 | | GQGTKLEIK |
| SEQ ID | anti-VB18 | DIVLTQSPATLSVSPGDRVTLSCRASQSISNFLHWYQQKPGQSPRLLIKYAS |
| NO: | humanized VL 1 | QSISGIPSRFSGSGSGTDFTLTINSLEPEDFAVYFCQQSNSWPLTFGQGTKLEI |
| 573 | | K |
| SEQ ID | anti-VB18 | EIVLTQSPATLSLSPGERATLSCRASQSISNFLHWYQQKPGQAPRLLIYYAS |
| NO: | humanized VL 2 | QSISGIPARFSGSGSGTDFTLTISSLEPEDFAVYYCQQSNSWPLTFGQGTKLEI |
| 574 | | K |
| SEQ ID | anti-VB18 | EIVLTQSPDFQSVTPKEKVTITCRASQSISNFLHWYQQKPDQSPKLLIKYASQ |
| NO: | humanized VL 3 | SISGVPSRFSGSGSGTDFTLTINSLEAEDAATYYCQQSNSWPLTFGQGTKLEI |
| 575 | | K |
| SEQ ID | anti-VB13.6 | DIQMTQSPSSLSASVGDTVTITCRTSENIYSNLVWYQQKPGKAPQLLIYAAT |
| NO: | humanized VL 1 | NLAEGVPSRFSGSGSGTDFSLTINSLQPEDFGTYYCQHFYGTPRTFGQGTKV |
| 576 | | EIK |
| SEQ ID | anti-VB13.6 | DIQMTQSPSSLSASVGDRVTITCRTSENIYSNLVWYQQKPGKAPKLLIYAAT |
| NO: | humanized VL 2 | NLAEGVPSRFSGSGSGTDFTLTISSLQPEDFATYYCQHFYGTPRTFGQGTKL |
| 577 | | EIK |
| SEQ ID | anti-VB16 | DIQMTQSPSSLSASVGDRVTLTCRASQDISGYLSWYQQKPGKAPKLLIYSTS |
| NO: | humanized VL-1 | TLDSGVPSRFSGSGSGTDFSLTISSLQSEDFATYYCLQYTTSPYTFGQGTKLE |
| 578 | 1 | IK |
| SEQ ID | anti-VB16 | DIQMTQSPSSLSASVGDRVTITCRASQDISGYLSWFQQKPGKAPKSLIYSTST |
| NO: | humanized VL-1 | LDSGVPSRFSGSGSGTDFTLTISSLQPEDFATYYCLQYTTSPYTFGQGTKLEI |
| 579 | 2 | K |
| SEQ ID | anti-VB16 | DIQMTQSPSSLSASVGDRVTITCRASQDISGYLSWYQQKPGKVPKLLIYSTS |
| NO: | humanized VL-1 | TLDSGVPSRFSGSGSGTDFTLTISSLQPEDVATYYCLQYTTSPYTFGQGTKL |
| 580 | 3 | EIK |
| SEQ ID | anti-VB16 | DIQMTQSPSTLSASVGDRVTITCKASDHINNWLAWYQQKPGKAPRLLIYGA |
| NO: | humanized VL-3 | TSLETGVPSRFSGSGSGTDFTLTITSLQPEDVATYYCQQYWSTPIFTFGQGT |
| 581 | 1 | KLEIK |
| SEQ ID | anti-VB16 | DIQMTQSPSSVSASVGDRVTITCKASDHINNWLAWYQQKPGKAPKLLIYG |
| NO: | humanized VL-3 | ATSLETGVPSRFSGSGSGTDFTLTISSLQPEDFATYYCQQYWSTPIFTFGQGT |
| 582 | 2 | KLEIK |
| SEQ ID | anti-VB16 | DIQMTQSPSSLSASVGDRVTITCKASDHINNWLAWYQQKPGKAPKSLIYGA |
| NO: | humanized VL-3 | TSLETGVPSRFSGSGSGTDFTLTISSLQPEDFATYYCQQYWSTPIFTFGQGTK |
| 583 | 3 | LEIK |
| SEQ ID | anti-VB20 VH | GFNIKDY |
| NO: | CDR1 | |
| 584 | | |
| SEQ ID | anti-VB20 VH | DPEDGE |
| NO: | CDR2 | |
| 585 | | |
| SEQ ID | anti-VB20 VH | SDLILFAY |
| NO: | CDR3 | |
| 586 | | |
| SEQ ID | anti-VB18 VH | GFDFSRY |
| NO: | CDR1 | |
| 587 | | |
| SEQ ID | anti-VB18 VH | NSDSST |
| NO: | CDR2 | |
| 588 | | |
| SEQ ID | anti-VB18 VH | PYYYGSSSWFAY |
| NO: | CDR3 | |
| 589 | | |
| SEQ ID | anti-VB13.6 VH | GYSFTGN |
| NO: | CDR1 | |
| 590 | | |
| SEQ ID | anti-VB13.6 VH | NPNTGG |
| NO: | CDR2 | |
| 591 | | |
| SEQ ID | anti-VB13.6 VH | SEYYGIYYYAMDY |
| NO: | CDR3 | |
| 592 | | |
| SEQ ID | anti-VB16 VH | GYTFTSY |
| NO: | CDR1 | |
| 593 | | |
| SEQ ID | anti-VB16 VH | NPSNGG |
| NO: | CDR2 | |
| 594 | | |
| SEQ ID | anti-VB16 VH | DSYYFDY |
| NO: | CDR3 | |
| 595 | | |
| SEQ ID | anti-VB20 VL | RSSQSIVHSNRYTYLE |
| NO: | CDR1 | |
| 596 | | |
| SEQ ID | anti-VB20 VL | GVSNRFS |
| NO: | CDR2 | |
| 597 | | |
| SEQ ID | anti-VB20 VL | FQGTHVPFM |
| NO: | CDR3 | |
| 598 | | |
| SEQ ID | anti-VB18 VL | RASQSISNFLH |
| NO: | CDR1 | |
| 599 | | |
| SEQ ID | anti-VB18 VL | YASQSIS |
| NO: | CDR2 | |
| 600 | | |
| SEQ ID | anti-VB18 VL | QQSNSWPLT |
| NO: | CDR3 | |
| 601 | | |
| SEQ ID | anti-VB13.6 VL | RTSENIYSNLV |
| NO: | CDR1 | |
| 602 | | |
| SEQ ID | anti-VB13.6 VL | AATNLAE |
| NO: | CDR2 | |
| 603 | | |
| SEQ ID | anti-VB13.6 VL | QHFYGTPRT |
| NO: | CDR3 | |
| 604 | | |
| SEQ ID | anti-VB16 VL | RASQDISGYLS |
| NO: | CDR1 | |
| 605 | | |
| SEQ ID | anti-VB16 VL | STSTLDS |
| NO: | CDR2 | |
| 606 | | |
| SEQ ID | anti-VB16 VL | LQYTTSPYT |
| NO: | CDR3 | |
| 607 | | |
| SEQ ID | anti-VB16 VL-3 | KASDHINNWLA |
| NO: | CDR1 | |
| 608 | | |
| SEQ ID | anti-VB16 VL-3 | GATSLET |
| NO: | CDR2 | |
| 609 | | |
| SEQ ID | anti-VB16 VL-3 | QQYWSTPIFT |
| NO: | CDR3 | |
| 610 | | |
| SEQ ID | VB20 murine VH | EVQLQQSGAELVKPGASVKLSCTASGFNIKDYYIHWVNQRSKQCLEWIGRI |
| NO: | | DPEDGETNYAPKFQGKATISADTSSNTAFLQLSSLTSEDTAVYYCARSDLIL |
| 611 | | FAYWGQGTLVTVSA |
| SEQ ID | VB20 murine VL | DVVMTQTPLSLPVSLGDQASISCRSSQSIVHSNRYTYLEWYLQKPGQSLKL |
| NO: | | LIYGVSNRFSGVPDRFSGSGSGTDFTLKISRVEAEDMGVYYCFQGTHVPFM |
| 612 | | FGCGTKLELK |
| SEQ ID | VB18 murine VH | EVKLLQSGGGLVQPGGSLKLSCAASGFDFSRYWMTWVRRAPGKCLEWIG |
| NO: | | EINSDSSTINYSPSLNDKFIISRDNAKNTLYLQMSKVRSEDTALYYCASPYY |
| 613 | | YGSSSWFAYWGQGTLVTVSA |
| SEQ ID | VB18 murine VL | DIVLTQSPATLSVTPGDRVSLSCRASQSISNFLHWYQQKSHESPRLLIKYAS |
| NO: | | QSISGIPSRFSGSGSGTDFTLSINSVETEDFGMYFCQQSNSWPLTFGCGTKLE |
| 614 | | LK |
| SEQ ID | VB13.6 murine | EVQLQQSGPELVKPGASVKISCKASGYSFTGNYMNWVKQSPEKCLEWIGEI |
| NO: | VH | NPNTGGTTYNQKLKAKATLTVDKSSSTAYMQLKSLTSEDSAVYYCARSEY |
| 615 | | YGIYYYAMDYWGQGTSVTVSS |
| SEQ ID | VB13.6 murine | DIQMTQSPASLSASVGETVTMTCRTSENIYSNLVWYQQKQGKSPQFLVYA |
| NO: | VL | ATNLAEGVPSRFSGSGSGTQFSLKINSLQPEDFGSYYCQHFYGTPRTFGCGT |
| 616 | | KLEIK |
| SEQ ID | VB16 murine VH | QVQLQQPGTELVKPGASVKLSCKASGYTFTSYWMHWVKQRPGQCLEWIG |
| NO: | | NINPSNGGTYYNEKFKTKATLTVDISSSTAYMQLSSLTSEDSAVYYCARDS |
| 617 | | YYFDYWGQGTTLAVSS |
| SEQ ID | VB16 murine VL- | DIQMTQSPSSLSASLGERVSLTCRASQDISGYLSWLQQKPDGTIKRLIYSTST |
| NO: | 1 | LDSGVPKRFSGSRSGSDYSLTISSLESEDFADYYCLQYTTSPYTFGCGTKLEI |
| 618 | | K |
| SEQ ID | VB16 murine VL- | DIQMIQSSSYLSVSLGDRVTITCKASDHINNWLAWYQQKPGNAPRLLISGA |
| NO: | 3 | TSLETGVPSRFSGSGSGKDYTLSITSLQTEDVATYYCQQYWSTPIFTFGCGT |
| 619 | | KLEIK |
| SEQ ID | anti-VB20 murine | EVQLQQSGAELVKPGASVKLSCTASGFNIKDYYIHWVNQRSKQGLEWIGRI |
| NO: | VH | DPEDGETNYAPKFQGKATISADTSSNTAFLQLSSLTSEDTAVYYCARSDLIL |
| 620 | | FAYWGQGTLVTVSA |
| SEQ ID | anti-VB20 murine | DVVMTQTPLSLPVSLGDQASISCRSSQSIVHSNRYTYLEWYLQKPGQSLKL |
| NO: | VL | LIYGVSNRFSGVPDRFSGSGSGTDFTLKISRVEAEDMGVYYCFQGTHVPFM |
| 621 | | FGAGTKLELK |
| SEQ ID | anti-VB18 murine | EVKLLQSGGGLVQPGGSLKLSCAASGFDFSRYWMTWVRRAPGKGLEWIG |
| NO: | VH | EINSDSSTINYSPSLNDKFIISRDNAKNTLYLQMSKVRSEDTALYYCASPYY |
| 622 | | YGSSSWFAYWGQGTLVTVSA |
| SEQ ID | anti-VB18 murine | DIVLTQSPATLSVTPGDRVSLSCRASQSISNFLHWYQQKSHESPRLLIKYAS |
| NO: | VL | QSISGIPSRFSGSGSGTDFTLSINSVETEDFGMYFCQQSNSWPLTFGAGTKLE |
| 623 | | LK |
| SEQ ID | anti-VB13.6 | EVQLQQSGPELVKPGASVKISCKASGYSFTGNYMNWVKQSPEKSLEWIGEI |
| NO: | murine VH | NPNTGGTTYNQKLKAKATLTVDKSSSTAYMQLKSLTSEDSAVYYCARSEY |
| 624 | | YGIYYYAMDYWGQGTSVTVSS |
| SEQ ID | anti-VB13.6 | DIQMTQSPASLSASVGETVTMTCRTSENIYSNLVWYQQKQGKSPQFLVYA |
| NO: | murine VL | ATNLAEGVPSRFSGSGSGTQFSLKINSLQPEDFGSYYCQHFYGTPRTFGGGT |
| 625 | | KLEIK |
| SEQ ID | anti-VB16 murine | QVQLQQPGTELVKPGASVKLSCKASGYTFTSYWMHWVKQRPGQGLEWIG |
| NO: | VH | NINPSNGGTYYNEKFKTKATLTVDISSSTAYMQLSSLTSEDSAVYYCARDS |
| 626 | | YYFDYWGQGTTLAVSS |
| SEQ ID | anti-VB16 murine | DIQMTQSPSSLSASLGERVSLTCRASQDISGYLSWLQQKPDGTIKRLIYSTST |
| NO: | VL-1 | LDSGVPKRFSGSRSGSDYSLTISSLESEDFADYYCLQYTTSPYTFGGGTKLEI |
| 627 | | K |
| SEQ ID | anti-VB16 murine | DIQMIQSSSYLSVSLGDRVTITCKASDHINNWLAWYQQKPGNAPRLLISGA |
| NO: | VL-3 | TSLETGVPSRFSGSGSGKDYTLSITSLQTEDVATYYCQQYWSTPIFTFGSGT |
| 628 | | KLEIK |
| SEQ ID | Full CAR murine | MLRLLLALNLFPSIQVTGQLQLVQSGPELREPGESVKISCKASGYTFTDYIV |
| NO: | Vβ1 | HWVKQAPGKCLKWMGWINTYTGTPTYADDFEGRFVFSLEASASTANLQIS |
| 629 | | NLKNEDTATYFCARSWRRGIRGIGFDWGQGVMVTVSSGSTSGSGKPGSGE |
| | | GSTKGDVQMTQSPYNLAASPGESVSINCKASKSINKYLAWYQQKPGKPNK |
| | | LLIYDGSTLQSGIPSRFSGSGSGTDFTLTIRGLEPEDFGLYYCQQHNEYPPTF |
| | | GCGTKLELKGTTTTPAPRPPTPAPTIASQPLSLRPEACRPAAGGAVHTRGLD |
| | | FACDRRPPSKPFWVLVVVGGVLACYSLLVTVAFIIFWVRKRGRKKLLYIFK |
| | | QPFMRPVQTTQEEDGCSCRFPEEEEGGCELASLRVKFSRSADAPAYQQGQN |
| | | QLYNELNLGRREEYDVLDKRRGRDPEMGGKPQRRKNPQEGLYNELQKDK |
| | | MAEAYSEIGMKGERRRGKGHDGLYQGLSTATKDTYDALHMQALPPRAS |
| SEQ ID | Full CAR murine | MLRLLLALNLFPSIQVTGQVHLQQSGAELARPGASVKMSCKASGYIFTDYT |
| NO: | Vβ7.2 | MHWVKQRPGQCLEWIGHINPSSGYSTYNQKFKDKATLTADKSSSTAYMQ |
| 630 | | LSSLTSEDSAVYYCARSLQLGRDYWGQGTTLTVSSGSTSGSGKPGSGEGST |
| | | KGDIVMTQSHKFMSTSVGDRVSITCKASQDVYTAVAWYQQKPGQSPKLLI |
| | | YSASNRYTGVPDRFTGSGSGTDFTFTISSVQAEDLAVYYCQQHYTTPRTFG |
| | | CGTKLEIKGTTTTPAPRPPTPAPTIASQPLSLRPEACRPAAGGAVHTRGLDF |
| | | ACDRRPPSKPFWVLVVVGGVLACYSLLVTVAFIIFWVRKRGRKKLLYIFKQ |
| | | PFMRPVQTTQEEDGCSCRFPEEEEGGCELASLRVKFSRSADAPAYQQGQNQ |
| | | LYNELNLGRREEYDVLDKRRGRDPEMGGKPQRRKNPQEGLYNELQKDKM |
| | | AEAYSEIGMKGERRRGKGHDGLYQGLSTATKDTYDALHMQALPPRAS |
| SEQ ID | Full CAR murine | MLRLLLALNLFPSIQVTGEVQLQQSGPELVKPGASVKISCKASGYSFTGNY |
| NO: | Vβ13.6 | MNWVKQSPEKCLEWIGEINPNTGGTTYNQKLKAKATLTVDKSSSTAYMQ |
| 631 | | LKSLTSEDSAVYYCARSEYYGIYYYAMDYWGQGTSVTVSSGSTSGSGKPG |
| | | SGEGSTKGDIQMTQSPASLSASVGETVTMTCRTSENIYSNLVWYQQKQGK |
| | | SPQFLVYAATNLAEGVPSRFSGSGSGTQFSLKINSLQPEDFGSYYCQHFYGT |
| | | PRTFGCGTKLEIKGTTTTPAPRPPTPAPTIASQPLSLRPEACRPAAGGAVHTR |
| | | GLDFACDRRPPSKPFWVLVVVGGVLACYSLLVTVAFIIFWVRKRGRKKLL |
| | | YIFKQPFMRPVQTTQEEDGCSCRFPEEEEGGCELASLRVKFSRSADAPAYQ |
| | | QGQNQLYNELNLGRREEYDVLDKRRGRDPEMGGKPQRRKNPQEGLYNEL |
| | | QKDKMAEAYSEIGMKGERRRGKGHDGLYQGLSTATKDTYDALHMQALPP |
| | | RAS |

What is claimed is:

1. A chimeric antigen receptor (CAR) comprising
(i) an antigen binding molecule that specifically binds to a TCR Vβ region, (ii) an extracellular domain, (iii) a transmembrane domain, (iv) a costimulatory domain and (v) an activating domain,
wherein the antigen binding molecule is a single chain variable fragment (scFv) comprising:
(a) a variable heavy chain (VH) according to any one of SEQ ID NOs: 45-131 and 534-544, and/or
(b) a variable light chain (VL) according to any one of SEQ ID NOs: 132-227 and 545-558.

2. The chimeric antigen receptor of claim 1, wherein
(a) (i) the variable heavy chain (VH) comprises CDR1, CDR2 and CDR3 according to SEQ ID NO:10, SEQ ID NO:11, and SEQ ID NO:12, respectively, and/or (ii) the variable light chain (VL) comprises CDR1, CDR2 and CDR3 according to SEQ ID NO:13, SEQ ID NO:14, SEQ ID NO:15, respectively;
(b) (i) the variable heavy chain (VH) comprises CDR1, CDR2 and CDR3 according to SEQ ID NO:370, SEQ ID NO:371, and SEQ ID NO:372, respectively, and/or (ii) the variable light chain (VL) comprises CDR1, CDR2 and CDR3 according to SEQ ID NO:418, SEQ ID NO:419, SEQ ID NO:420, respectively;
(c) (i) the variable heavy chain (VH) comprises CDR1, CDR2 and CDR3 according to SEQ ID NO:373, SEQ ID NO:374, and SEQ ID NO:375 respectively, and/or (ii) the variable light chain (VL) comprises CDR1, CDR2 and CDR3 according to SEQ ID NO:421, SEQ ID NO:422, SEQ ID NO:423, respectively;
(d) (i) the variable heavy chain (VH) comprises CDR1, CDR2 and CDR3 according to SEQ ID NO:376, SEQ ID NO:377, and SEQ ID NO:378, respectively, and/or (ii) the variable light chain (VL) comprises CDR1, CDR2 and CDR3 according to SEQ ID NO:424, SEQ ID NO:425, SEQ ID NO:426, respectively;
(e) (i) the variable heavy chain (VH) comprises CDR1, CDR2 and CDR3 according to SEQ ID NO:379, SEQ ID NO:380, and SEQ ID NO:381 respectively, and/or (ii) the variable light chain (VL) comprises CDR1, CDR2 and CDR3 according to SEQ ID NO:427, SEQ ID NO:428, SEQ ID NO:429, respectively;
(f) (i) the variable heavy chain (VH) comprises CDR1, CDR2 and CDR3 according to SEQ ID NO:382, SEQ ID NO:383, and SEQ ID NO:384, respectively, and/or (ii) the variable light chain (VL) comprises CDR1, CDR2 and CDR3 according to SEQ ID NO:430, SEQ ID NO:431, SEQ ID NO:432, respectively;
(g) (i) the variable heavy chain (VH) comprises CDR1, CDR2 and CDR3 according to SEQ ID NO:385, SEQ ID NO:386, and SEQ ID NO:387, respectively, and/or (ii) the variable light chain (VL) comprises CDR1, CDR2 and CDR3 according to SEQ ID NO:433, SEQ ID NO:434, SEQ ID NO:435, respectively;
(h) (i) the variable heavy chain (VH) comprises CDR1, CDR2 and CDR3 according to SEQ ID NO:388, SEQ ID NO:389, and SEQ ID NO:390, respectively, and/or (ii) the variable light chain (VL) comprises CDR1, CDR2 and CDR3 according to SEQ ID NO:436, SEQ ID NO:437, SEQ ID NO:438, respectively;
(i) (i) the variable heavy chain (VH) comprises CDR1, CDR2 and CDR3 according to SEQ ID NO:391, SEQ ID NO:392, and SEQ ID NO:393 respectively, and/or (ii) the variable light chain (VL) comprises CDR1, CDR2 and CDR3 according to SEQ ID NO:439, SEQ ID NO:440, SEQ ID NO:441, respectively;
(j) (i) the variable heavy chain (VH) comprises CDR1, CDR2 and CDR3 according to SEQ ID NO:394, SEQ ID NO:395, and SEQ ID NO:396, respectively, and/or (ii) the variable light chain (VL) comprises CDR1, CDR2 and CDR3 according to SEQ ID NO:442, SEQ ID NO:443, SEQ ID NO:444, respectively;
(k) (i) the variable heavy chain (VH) comprises CDR1, CDR2 and CDR3 according to SEQ ID NO:397, SEQ ID NO:398, and SEQ ID NO:399, respectively, and/or (ii) the variable light chain (VL) comprises CDR1, CDR2 and CDR3 according to SEQ ID NO:445, SEQ ID NO:446, SEQ ID NO:447, respectively;
(l) (i) the variable heavy chain (VH) comprises CDR1, CDR2 and CDR3 according to SEQ ID NO:400, SEQ ID NO:401, and SEQ ID NO:402, respectively, and/or (ii) the variable light chain (VL) comprises CDR1, CDR2 and CDR3 according to SEQ ID NO:448, SEQ ID NO:449, SEQ ID NO:450, respectively;
(m) (i) the variable heavy chain (VH) comprises CDR1, CDR2 and CDR3 according to SEQ ID NO:403, SEQ ID NO:404, and SEQ ID NO:405 respectively, and/or (ii) the variable light chain (VL) comprises CDR1, CDR2 and CDR3 according to SEQ ID NO:451, SEQ ID NO:452, SEQ ID NO:453, respectively;
(n) (i) the variable heavy chain (VH) comprises CDR1, CDR2 and CDR3 according to SEQ ID NO:406, SEQ ID NO:407, and SEQ ID NO:408, respectively, and/or (ii) the variable light chain (VL) comprises CDR1, CDR2 and CDR3 according to SEQ ID NO:454, SEQ ID NO:455, SEQ ID NO:456, respectively;
(o) (i) the variable heavy chain (VH) comprises CDR1, CDR2 and CDR3 according to SEQ ID NO:409, SEQ ID NO:410, and SEQ ID NO:411, respectively, and/or (ii) the variable light chain (VL) comprises CDR1, CDR2 and CDR3 according to SEQ ID NO:457, SEQ ID NO:458, SEQ ID NO:459, respectively;
(p) (i) the variable heavy chain (VH) comprises CDR1, CDR2 and CDR3 according to SEQ ID NO:412, SEQ ID NO:413, and SEQ ID NO:414, respectively, and/or (ii) the variable light chain (VL) comprises CDR1, CDR2 and CDR3 according to SEQ ID NO:460, SEQ ID NO:461, SEQ ID NO:462, respectively;
(q) (i) the variable heavy chain (VH) comprises CDR1, CDR2 and CDR3 according to SEQ ID NO:415, SEQ ID NO:416, and SEQ ID NO:417, respectively, and/or (ii) the variable light chain (VL) comprises CDR1, CDR2 and CDR3 according to SEQ ID NO:463, SEQ ID NO:464, SEQ ID NO:465, respectively;
(r) (i) the variable heavy chain (VH) comprises CDR1, CDR2 and CDR3 according to SEQ ID NO:584, SEQ ID NO:585, and SEQ ID NO:586, respectively, and/or (ii) the variable light chain (VL) comprises CDR1, CDR2 and CDR3 according to SEQ ID NO:596, SEQ ID NO:597, SEQ ID NO:598, respectively;
(s) (i) the variable heavy chain (VH) comprises CDR1, CDR2 and CDR3 according to SEQ ID NO:587, SEQ ID NO:588, and SEQ ID NO:589, respectively, and/or (ii) the variable light chain (VL) comprises CDR1, CDR2 and CDR3 according to SEQ ID NO:599, SEQ ID NO:601, SEQ ID NO:601, respectively;
(t) (i) the variable heavy chain (VH) comprises CDR1, CDR2 and CDR3 according to SEQ ID NO:590, SEQ ID NO:591, and SEQ ID NO:592, respectively, and/or (ii) the variable light chain (VL) comprises CDR1, CDR2 and CDR3 according to SEQ ID NO:602, SEQ ID NO:603, SEQ ID NO:604, respectively; or
(u) (i) the variable heavy chain (VH) comprises CDR1, CDR2 and CDR3 according to SEQ ID NO:593, SEQ ID NO:594, and SEQ ID NO:595, respectively, and/or (ii) the variable light chain (VL) comprises CDR1, CDR2 and CDR3 according to SEQ ID NO:605, SEQ ID NO:606, SEQ ID NO:607, respectively, or SEQ ID NO: 608, SEQ ID NO: 609, SEQ ID NO: 610, respectively.

3. The chimeric antigen receptor of claim 1 or 2, wherein the variable heavy chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NOs: 45-131 and 534-544.

4. The chimeric antigen receptor of claim 1 or 2, wherein the variable light chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NO: 132-227 and 545-558.

5. The chimeric antigen receptor of any one of claim 1-4, wherein the TCR Vβ region is selected from the group consisting of Vβ, Vβ2, Vβ3, Vβ4, Vβ5.1, Vβ5.3, Vβ7.1, Vβ7.2, Vβ8, Vβ9, Vβ11, Vβ12, Vβ13.1, Vβ13.2, Vβ13.6, Vβ14, Vβ16, Vβ17, Vβ18, Vβ20, and Vβ22.

6. The chimeric antigen receptor of claim 5, wherein the TCR Vβ region is Vβ2.

7. The chimeric antigen receptor of claim 1, wherein the antigen-binding domain is a scFv.

8. The chimeric antigen receptor of claim 7, wherein the scFv comprises an amino acid sequence according to any of SEQ ID NOs: 14-44.

9. The chimeric antigen receptor of any one of claim 1-8, wherein the costimulatory domain comprises a 4-1BB costimulatory domain.

10. The chimeric antigen receptor of claim 9, wherein the 4-1BB costimulatory domain comprises an amino acid sequence according to SEQ ID NO: 8.

11. The chimeric antigen receptor of any one of claim 1-10, wherein the transmembrane domain comprises an hCD28 transmembrane.

12. The chimeric antigen receptor of claim 11, wherein the transmembrane domain comprises a hCD28 amino acid sequence according to SEQ ID NO: 7.

13. The chimeric antigen receptor of any one of claim 1-12, wherein the signaling domain comprises a CD247 signaling domain.

14. The chimeric antigen receptor of claim 13, wherein the signaling domain comprises a CD247 signaling domain amino acid sequence according to SEQ ID NO: 9.

15. The chimeric antigen receptor of any one of claim 1-14, wherein the scFv comprises a CD28 signal peptide.

16. The chimeric antigen receptor of claim 15, wherein the scFv comprises a CD28 signal peptide an amino acid sequence according to SEQ ID NO:3.

17. The chimeric antigen receptor of any one of claim 1-16, wherein the scFv comprises a linker peptide.

18. The chimeric antigen receptor of claim 17, wherein the scFv comprises a linker peptide according to SEQ ID NO: 4.

19. An isolated polynucleotide comprising a nucleic acid molecule encoding the VH of the chimeric antigen receptor of any one of claim 1-18.

20. The isolated polynucleotide of claim 19 further comprising a nucleic acid molecule encoding the VL of the chimeric antigen receptor of any one of claim 1-18.

21. An isolated polynucleotide comprising a nucleic acid molecule encoding the VL of the chimeric antigen receptor thereof of any one of claim 1-18.

22. The isolated polynucleotide of claim 21 further comprising a nucleic acid molecule encoding the VH of the chimeric antigen receptor of any one of claim 1-18.

23. An isolated polynucleotide comprising a nucleic acid molecule encoding the chimeric antigen receptor of any one of claim 1-18.

24. A vector comprising the isolated polynucleotide according to claim 23.

25. The vector of claim 24, wherein the vector is selected from the group consisting of a retroviral vector, an adenoviral vector, a lentiviral vector, and an adeno-associated viral vector.

26. An antibody or antigen-binding fragment thereof capable of specifically binding to a TCR Vβ region, comprising a heavy chain variable region (VH) having an amino acid sequence according to any of SEQ ID NOs: 228-295 and 559-569.

27. An antibody or antigen-binding fragment thereof capable of specifically binding to a TCR Vβ region, comprising a light chain variable region (VL) having an amino acid sequence according to any of SEQ ID NOs: 296-369 and 570-583.

28. The antibody or antigen-binding fragment of claim 26, wherein the VH comprises an amino acid sequence having at least 85%, at least 90%, at least 91%, at least 92%, at least 93%, at least 94%, at least 95%, at least 96%, at least 97%, at least 98%, at least 99%, or about 100% sequence identity to any of SEQ ID NOs: 228-295 and 559-569.

29. The antibody or antigen-binding fragment of claim 27, wherein the VL comprises an amino acid sequence having at least 85%, at least 90%, at least 91%, at least 92%, at least 93%, at least 94%, at least 95%, at least 96%, at least 97%, at least 98%, at least 99%, or about 100% sequence identity to any of SEQ ID NOs: 296-369 and 570-583.

30. The antibody or antigen-binding fragment of claim 26, wherein the antibody or antigen-binding fragment further comprises a light chain variable region (VL) having an amino acid sequence according to any of SEQ ID NOs: 296-369 and 570-583.

31. The antibody or antigen-binding fragment of claim 27, wherein the antibody or antigen-binding fragment further comprises a heavy chain variable region (VH) having an amino acid sequence according to any of SEQ ID NOs: 228-295 and 559-569.

32. The antibody or antigen-binding fragment of any one of claim 26-31, wherein the antibody or antigen-binding fragment thereof is human, humanized, or chimeric.

33. The antibody or antigen-binding fragment of any one of claim 26-32, wherein the antibody or antigen-binding fragment thereof is an IgG antibody.

34. The antibody or antigen-binding fragment of claim 33, wherein the IgG antibody is an IgG1 antibody or an IgG4 antibody.

35. The antibody or antigen-binding fragment of any one of claim 26-34, wherein the antibody is an antigen-binding fragment of an antibody.

36. The antibody or antigen-binding fragment of claim 35, wherein the fragment is selected from the group consisting of Fab, F(ab′)2, Fv, scFv, scFv-Fc, dsFv and a single domain molecule.

37. The antibody or antigen-binding fragment claim 35 or 36, wherein the fragment is a scFv.

38. The antibody or antigen-binding fragment claim 35 or 36, wherein the fragment is a Fab.

39. The antibody or antigen-binding fragment claim 35 or 36, wherein the fragment is an intrabody.

40. The antibody or antigen-binding fragment of any one of claim 26-39, wherein said antigen-binding fragment is devoid of an Fc region.

41. The antibody or antigen-binding fragment of any one of claim 26-40, comprising a VH and a VL on the same polypeptide chain.

42. The antibody or antigen-binding fragment of any one of claim 26-41, wherein the VH and VL are connected by a linker.

43. The antibody or antigen-binding fragment of any of claim 26-42, wherein the TCR Vβ region is selected from the group consisting of Vβ1, Vβ2, Vβ3, Vβ4, Vβ5.1, Vβ5.3, Vβ7.1, Vβ7.2, Vβ8, Vβ9, Vβ11, Vβ12, Vβ13.1, Vβ13.2, Vβ13.6, Vβ14, Vβ16, Vβ17, Vβ18, Vβ20, and Vβ22.

44. The antibody or antigen-binding fragment of claim 43, wherein the TCR Vβ region is Vβ2.

45. A method for treating cancer in a subject in need thereof, the method comprising: administering to the subject a therapeutically effective amount of a T-cell composition comprising a CAR, wherein the CAR comprises (i) an antigen binding molecule that specifically binds to a TCR Vβ region, (ii) an extracellular domain, (iii) a transmembrane domain, (iv) a costimulatory domain and (v) an activating domain,
wherein the antigen binding molecule is a single chain variable fragment (scFv) comprising:
(a) a variable heavy chain according to any one of SEQ ID NOs: 45-131 and 534-544, and/or
(b) a variable light chain according to any one of SEQ ID NOs: 132-227 and 545-558.

46. The method of claim 45, wherein the chimeric antigen receptor comprises (a) (i) the variable heavy chain (VH) comprises CDR1, CDR2 and CDR3 according to SEQ ID NO:10, SEQ ID NO:11, and SEQ ID NO:12, respectively, and/or (ii) the variable light chain (VL) comprises CDR1, CDR2 and CDR3 according to SEQ ID NO:13, SEQ ID NO:14, SEQ ID NO:15, respectively;
(b) (i) the variable heavy chain (VH) comprises CDR1, CDR2 and CDR3 according to SEQ ID NO:370, SEQ ID NO:371, and SEQ ID NO:372, respectively, and/or (ii) the variable light chain (VL) comprises CDR1, CDR2 and CDR3 according to SEQ ID NO:418, SEQ ID NO:419, SEQ ID NO:420, respectively;
(c) (i) the variable heavy chain (VH) comprises CDR1, CDR2 and CDR3 according to SEQ ID NO:373, SEQ ID NO:374, and SEQ ID NO:375 respectively, and/or (ii) the variable light chain (VL) comprises CDR1, CDR2 and CDR3 according to SEQ ID NO:421, SEQ ID NO:422, SEQ ID NO:423, respectively;
(d) (i) the variable heavy chain (VH) comprises CDR1, CDR2 and CDR3 according to SEQ ID NO:376, SEQ ID NO:377, and SEQ ID NO:378, respectively, and/or (ii) the variable light chain (VL) comprises CDR1, CDR2 and CDR3 according to SEQ ID NO:424, SEQ ID NO:425, SEQ ID NO:426, respectively;
(e) (i) the variable heavy chain (VH) comprises CDR1, CDR2 and CDR3 according to SEQ ID NO:379, SEQ ID NO:380, and SEQ ID NO:381 respectively, and/or (ii) the variable light chain (VL) comprises CDR1, CDR2 and CDR3 according to SEQ ID NO:427, SEQ ID NO:428, SEQ ID NO:429, respectively;
(f) (i) the variable heavy chain (VH) comprises CDR1, CDR2 and CDR3 according to SEQ ID NO:382, SEQ ID NO:383, and SEQ ID NO:384, respectively, and/or (ii) the variable light chain (VL) comprises CDR1, CDR2 and CDR3 according to SEQ ID NO:430, SEQ ID NO:431, SEQ ID NO:432, respectively;
(g) (i) the variable heavy chain (VH) comprises CDR1, CDR2 and CDR3 according to SEQ ID NO:385, SEQ ID NO:386, and SEQ ID NO:387, respectively, and/or (ii) the variable light chain (VL) comprises CDR1, CDR2 and CDR3 according to SEQ ID NO:433, SEQ ID NO:434, SEQ ID NO:435, respectively;
(h) (i) the variable heavy chain (VH) comprises CDR1, CDR2 and CDR3 according to SEQ ID NO:388, SEQ ID NO:389, and SEQ ID NO:390, respectively, and/or (ii) the variable light chain (VL) comprises CDR1, CDR2 and CDR3 according to SEQ ID NO:436, SEQ ID NO:437, SEQ ID NO:438, respectively;
(i) (i) the variable heavy chain (VH) comprises CDR1, CDR2 and CDR3 according to SEQ ID NO:391, SEQ ID NO:392, and SEQ ID NO:393 respectively, and/or
(ii) the variable light chain (VL) comprises CDR1, CDR2 and CDR3 according to SEQ ID NO:439, SEQ ID NO:440, SEQ ID NO:441, respectively;
(j) (i) the variable heavy chain (VH) comprises CDR1, CDR2 and CDR3 according to SEQ ID NO:394, SEQ ID NO:395, and SEQ ID NO:396, respectively, and/or (ii) the variable light chain (VL) comprises CDR1, CDR2 and CDR3 according to SEQ ID NO:442, SEQ ID NO:443, SEQ ID NO:444, respectively;
(k) (i) the variable heavy chain (VH) comprises CDR1, CDR2 and CDR3 according to SEQ ID NO:397, SEQ ID NO:398, and SEQ ID NO:399, respectively, and/or (ii) the variable light chain (VL) comprises CDR1, CDR2 and CDR3 according to SEQ ID NO:445, SEQ ID NO:446, SEQ ID NO:447, respectively;
(l) (i) the variable heavy chain (VH) comprises CDR1, CDR2 and CDR3 according to SEQ ID NO:400, SEQ ID NO:401, and SEQ ID NO:402, respectively, and/or (ii) the variable light chain (VL) comprises CDR1, CDR2 and CDR3 according to SEQ ID NO:448, SEQ ID NO:449, SEQ ID NO:450, respectively;
(m) (i) the variable heavy chain (VH) comprises CDR1, CDR2 and CDR3 according to SEQ ID NO:403, SEQ ID NO:404, and SEQ ID NO:405 respectively, and/or (ii) the variable light chain (VL) comprises CDR1, CDR2 and CDR3 according to SEQ ID NO:451, SEQ ID NO:452, SEQ ID NO:453, respectively;
(n) (i) the variable heavy chain (VH) comprises CDR1, CDR2 and CDR3 according to SEQ ID NO:406, SEQ ID NO:407, and SEQ ID NO:408, respectively, and/or (ii) the variable light chain (VL) comprises CDR1, CDR2 and CDR3 according to SEQ ID NO:454, SEQ ID NO:455, SEQ ID NO:456, respectively;
(o) (i) the variable heavy chain (VH) comprises CDR1, CDR2 and CDR3 according to SEQ ID NO:409, SEQ ID NO:410, and SEQ ID NO:411, respectively, and/or (ii) the variable light chain (VL) comprises CDR1, CDR2 and CDR3 according to SEQ ID NO:457, SEQ ID NO:458, SEQ ID NO:459, respectively;
(p) (i) the variable heavy chain (VH) comprises CDR1, CDR2 and CDR3 according to SEQ ID NO:412, SEQ ID NO:413, and SEQ ID NO:414, respectively, and/or (ii) the variable light chain (VL) comprises CDR1, CDR2 and CDR3 according to SEQ ID NO:460, SEQ ID NO:461, SEQ ID NO:462, respectively;
(q) (i) the variable heavy chain (VH) comprises CDR1, CDR2 and CDR3 according to SEQ ID NO:415, SEQ ID NO:416, and SEQ ID NO:417, respectively, and/or (ii) the variable light chain (VL) comprises CDR1, CDR2 and CDR3 according to SEQ ID NO:463, SEQ ID NO:464, SEQ ID NO:465, respectively;
(r) (i) the variable heavy chain (VH) comprises CDR1, CDR2 and CDR3 according to SEQ ID NO:584, SEQ ID NO:585, and SEQ ID NO:586, respectively, and/or (ii) the variable light chain (VL) comprises CDR1, CDR2 and CDR3 according to SEQ ID NO:596, SEQ ID NO:597, SEQ ID NO:598, respectively;
(s) (i) the variable heavy chain (VH) comprises CDR1, CDR2 and CDR3 according to SEQ ID NO:587, SEQ ID NO:588, and SEQ ID NO:589, respectively, and/or (ii) the variable light chain (VL) comprises CDR1, CDR2 and CDR3 according to SEQ ID NO:599, SEQ ID NO:601, SEQ ID NO:601, respectively;
(t) (i) the variable heavy chain (VH) comprises CDR1, CDR2 and CDR3 according to SEQ ID NO:590, SEQ ID NO:591, and SEQ ID NO:592, respectively, and/or (ii) the variable light chain (VL) comprises CDR1, CDR2 and CDR3 according to SEQ ID NO:602, SEQ ID NO:603, SEQ ID NO:604, respectively; or
(u) (i) the variable heavy chain (VH) comprises CDR1, CDR2 and CDR3 according to SEQ ID NO:593, SEQ ID NO:594, and SEQ ID NO:595, respectively, and/or (ii) the variable light chain (VL) comprises CDR1, CDR2 and CDR3 according to SEQ ID NO:605, SEQ ID NO:606, SEQ ID NO:607, respectively, or SEQ ID NO: 608, SEQ ID NO: 609, SEQ ID NO: 610, respectively.

47. The method of claim 45 or 46, wherein the variable heavy chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NOs: 45-131 and 534-544.

48. The method of claim 45 or 46, wherein the variable light chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NO: 132-227 and 545-558.

49. The method of any one of claim 45-48, wherein the TCR Vβ region is selected from the group consisting of Vβ1, Vβ2, Vβ3, Vβ4, Vβ5.1, Vβ5.3, Vβ7.1, Vβ7.2, Vβ8, Vβ9, Vβ11, Vβ12, Vβ13.1, Vβ13.2, Vβ13.6, Vβ14, Vβ16, Vβ17, Vβ18, Vβ20, and Vβ22.

50. The method of claim 49, wherein the TCR Vβ region is Vβ2.

51. The method of any one of claim 45-50, wherein the cancer is selected from the group consisting of T-cell Lymphoma, T-cell Leukemia, Cutaneous T-cell Lymphoma, Peripheral T-cell Lymphoma (PTCL), Not Otherwise Specified PTCL (PTCL-NOS), Angioimmunoblastic T-cell Lymphoma (AITL), Anaplastic Large-cell Lymphoma (ALCL), Enteropathy-Associated T-cell Lymphoma (EATL), Adult T-cell Leukemia/Lymphoma (ATLL), Hepatosplenic T-cell Lymphoma (HSTL), Subcutaneous Panniculitis-Like T-cell Lymphoma (SPTCL), T-cell Acute Lymphoblastic Leukemia (T-ALL), T-cell Chronic Lymphocytic Leukemia, Large Granular Lymphocyte Leukemia, T-cell Prolymphocytic Leukemia, Lympomatoid Papulosis, Small Medium Pleiomorphic T-cell Lymphoma, Mycosis Fungoides, Sezary Syndrome, and Cytotoxic T-cell Lymphoma.

52. The method of any one of claim 45-51, wherein the composition is delivered intratumorally, intravenously, intraperitoneally, intramuscularly, intrathecally, transdermally, or subcutaneously.

53. The method of claim 52, wherein the composition is delivered intravenously or intratumorally.

54. The method of any one of claim 45-53, wherein the subject is a human.

55. A method for treating cancer in a subject in need thereof, the method comprising: administering to the subject a therapeutically effective amount of a composition comprising an antibody or antigen-binding fragment thereof capable of specifically binding to a TCR Vβ region, comprising a (i) heavy chain variable region (VH) having an amino acid sequence according to any of SEQ ID NOs: 228-295 and 559-569, or
(ii) a light chain variable region (VL) having an amino acid sequence according to any of SEQ ID NOs: 296-369 and 570-583.

56. The method of claim 55, wherein the antibody or antigen-binding fragment comprises
(i) a variable heavy chain (VH) which comprises:
(a) CDR1, CDR2 and CDR3 according to SEQ ID NO:10, SEQ ID NO:11, and SEQ ID NO:12, respectively;
(b) CDR1, CDR2 and CDR3 according to SEQ ID NO:370, SEQ ID NO:371, and SEQ ID NO:372, respectively;
(c) CDR1, CDR2 and CDR3 according to SEQ ID NO:373, SEQ ID NO:374, and SEQ ID NO:375 respectively;
(d) CDR1, CDR2 and CDR3 according to SEQ ID NO:376, SEQ ID NO:377, and SEQ ID NO:378, respectively;
(e) CDR1, CDR2 and CDR3 according to SEQ ID NO:379, SEQ ID NO:380, and SEQ ID NO:381 respectively;
(f) CDR1, CDR2 and CDR3 according to SEQ ID NO:382, SEQ ID NO:383, and SEQ ID NO:384, respectively;
(g) CDR1, CDR2 and CDR3 according to SEQ ID NO:385, SEQ ID NO:386, and SEQ ID NO:387, respectively;
(h) CDR1, CDR2 and CDR3 according to SEQ ID NO:388, SEQ ID NO:389, and SEQ ID NO:390, respectively;
(i) CDR1, CDR2 and CDR3 according to SEQ ID NO:391, SEQ ID NO:392, and SEQ ID NO:393, respectively;
(j) CDR1, CDR2 and CDR3 according to SEQ ID NO:394, SEQ ID NO:395, and SEQ ID NO:396, respectively;
(k) CDR1, CDR2 and CDR3 according to SEQ ID NO:397, SEQ ID NO:398, and SEQ ID NO:399, respectively;
(l) CDR1, CDR2 and CDR3 according to SEQ ID NO:400, SEQ ID NO:401, and SEQ ID NO:402, respectively;
(m) CDR1, CDR2 and CDR3 according to SEQ ID NO:403, SEQ ID NO:404, and SEQ ID NO:405, respectively;
(n) CDR1, CDR2 and CDR3 according to SEQ ID NO:406, SEQ ID NO:407, and SEQ ID NO:408, respectively;
(o) CDR1, CDR2 and CDR3 according to SEQ ID NO:409, SEQ ID NO:410, and SEQ ID NO:411, respectively;
(p) CDR1, CDR2 and CDR3 according to SEQ ID NO:412, SEQ ID NO:413, and SEQ ID NO:414, respectively;
(q) CDR1, CDR2 and CDR3 according to SEQ ID NO:415, SEQ ID NO:416, and SEQ ID NO:417, respectively;
(r) CDR1, CDR2 and CDR3 according to SEQ ID NO:584, SEQ ID NO:585, and SEQ ID NO:586, respectively;
(s) CDR1, CDR2 and CDR3 according to SEQ ID NO:587, SEQ ID NO:588, and SEQ ID NO:589, respectively;
(t) CDR1, CDR2 and CDR3 according to SEQ ID NO:590, SEQ ID NO:591, and SEQ ID NO:592, respectively; or
(u) CDR1, CDR2 and CDR3 according to SEQ ID NO:593, SEQ ID NO:594, and SEQ ID NO:595, respectively; and/or
(ii) a variable light chain (VL) which comprises:
(a) CDR1, CDR2 and CDR3 according to SEQ ID NO:13, SEQ ID NO:14, SEQ ID NO:15,
(b) CDR1, CDR2 and CDR3 according to SEQ ID NO:418, SEQ ID NO:419, SEQ ID NO:420, respectively;
(c) CDR1, CDR2 and CDR3 according to SEQ ID NO:421, SEQ ID NO:422, SEQ ID NO:423, respectively;
(d) CDR1, CDR2 and CDR3 according to SEQ ID NO:424, SEQ ID NO:425, SEQ ID NO:426, respectively;
(e) CDR1, CDR2 and CDR3 according to SEQ ID NO:427, SEQ ID NO:428, SEQ ID NO:429, respectively;
(f) CDR1, CDR2 and CDR3 according to SEQ ID NO:430, SEQ ID NO:431, SEQ ID NO:432, respectively;
(g) CDR1, CDR2 and CDR3 according to SEQ ID NO:433, SEQ ID NO:434, SEQ ID NO:435, respectively;
(h) CDR1, CDR2 and CDR3 according to SEQ ID NO:436, SEQ ID NO:437, SEQ ID NO:438, respectively;
(i) CDR1, CDR2 and CDR3 according to SEQ ID NO:439, SEQ ID NO:440, SEQ ID NO:441, respectively;
(j) CDR1, CDR2 and CDR3 according to SEQ ID NO:442, SEQ ID NO:443, SEQ ID NO:444, respectively;
(k) CDR1, CDR2 and CDR3 according to SEQ ID NO:445, SEQ ID NO:446, SEQ ID NO:447, respectively;
(l) CDR1, CDR2 and CDR3 according to SEQ ID NO:448, SEQ ID NO:449, SEQ ID NO:450, respectively;
(m) CDR1, CDR2 and CDR3 according to SEQ ID NO:451, SEQ ID NO:452, SEQ ID NO:453, respectively;
(n) CDR1, CDR2 and CDR3 according to SEQ ID NO:454, SEQ ID NO:455, SEQ ID NO:456, respectively;
(o) CDR1, CDR2 and CDR3 according to SEQ ID NO:457, SEQ ID NO:458, SEQ ID NO:459, respectively;
(p) CDR1, CDR2 and CDR3 according to SEQ ID NO:460, SEQ ID NO:461, SEQ ID NO:462, respectively;
(q) CDR1, CDR2 and CDR3 according to SEQ ID NO:463, SEQ ID NO:464, SEQ ID NO:465, respectively;
(r) CDR1, CDR2 and CDR3 according to SEQ ID NO:596, SEQ ID NO:597, SEQ ID NO:598, respectively;
(s) CDR1, CDR2 and CDR3 according to SEQ ID NO:599, SEQ ID NO:601, SEQ ID NO:601, respectively;
(t) CDR1, CDR2 and CDR3 according to SEQ ID NO:602, SEQ ID NO:603, SEQ ID NO:604, respectively; or
(u) CDR1, CDR2 and CDR3 according to SEQ ID NO:605, SEQ ID NO:606, SEQ ID NO:607, respectively, or SEQ ID NO: 608, SEQ ID NO: 609, SEQ ID NO: 610, respectively.

57. The method of claim 55 or 56, wherein the variable heavy chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NOs: 228-295 and 559-569.

58. The method of claim 55 or 56, wherein the variable light chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NO: 296-369 and 570-583.

59. The antibody or antigen-binding fragment of claim 55, wherein the antibody or antigen-binding fragment further comprises a light chain variable region (VL) having an amino acid sequence according to any of SEQ ID NOs: 296-369 and 570-583.

60. The antibody or antigen-binding fragment of claim 55, wherein the antibody or antigen-binding fragment further comprises a heavy chain variable region (VH) having an amino acid sequence according to any of SEQ ID NOs: 228-295 and 534-544.

61. The method of any one of claim 55-60, wherein the TCR Vβ region is selected from the group consisting of Vβ1, Vβ2, Vβ3, Vβ4, Vβ5.1, Vβ5.3, Vβ7.1, Vβ7.2, Vβ8, Vβ9, Vβ11, Vβ12, Vβ13.1, Vβ13.2, Vβ13.6, Vβ14, Vβ16, Vβ17, Vβ18, Vβ20, and Vβ22.

62. The method of claim 61, wherein the TCR Vβ region is Vβ2.

63. The method of any one of claim 55-62, wherein the cancer is selected from the group consisting of T-cell Lymphoma, T-cell Leukemia, Cutaneous T-cell Lymphoma, Peripheral T-cell Lymphoma (PTCL), Not Otherwise Specified PTCL (PTCL-NOS), Angioimmunoblastic T-cell Lymphoma (AITL), Anaplastic Large-cell Lymphoma (ALCL), Enteropathy-Associated T-cell Lymphoma (EATL), Adult T-cell Leukemia/Lymphoma (ATLL), Hepatosplenic T-cell Lymphoma (HSTL), Subcutaneous Panniculitis-Like T-cell Lymphoma (SPTCL), T-cell Acute Lymphoblastic Leukemia (T-ALL), T-cell Chronic Lymphocytic Leukemia, Large Granular Lymphocyte Leukemia, T-cell Prolymphocytic Leukemia, Lympomatoid Papulosis, Small Medium Pleiomorphic T-cell Lymphoma, Mycosis Fungoides, Sezary Syndrome, and Cytotoxic T-cell Lymphoma.

64. The method of any one of claim 55-63, wherein the composition is delivered intratumorally, intravenously, intraperitoneally, intramuscularly, intrathecally, transdermally, or subcutaneously.

65. The method of claim 64, wherein the composition is delivered intravenously.

66. The method of claim 64, wherein the composition is delivered intratumorally.

67. The method of any one of claim 55-66, wherein the subject is a human.

68. A method for treating T-cell mediated diseases in a subject in need thereof, the method comprising: administering to the subject a therapeutically effective amount of a T-cell composition comprising a CAR, wherein the CAR comprises (i) an antigen binding molecule that specifically binds to a TCR Vβ region, (ii) an extracellular domain, (iii) a transmembrane domain, (iv) a costimulatory domain and (v) an activating domain,
wherein the antigen binding molecule is a single chain variable fragment (scFv) comprising:
(a) a variable heavy chain according to any one of SEQ ID NOs: 45-131 and 534-544, and/or
(b) a variable light chain according to any one of SEQ ID NOs: 132-227 and 545-558.

69. The method of claim 68, wherein the chimeric antigen receptor comprises
(a) (i) the variable heavy chain (VH) comprises CDR1, CDR2 and CDR3 according to SEQ ID NO:10, SEQ ID NO:11, and SEQ ID NO:12, respectively, and/or (ii) the variable light chain (VL) comprises CDR1, CDR2 and CDR3 according to SEQ ID NO:13, SEQ ID NO:14, SEQ ID NO:15, respectively;
(b) (i) the variable heavy chain (VH) comprises CDR1, CDR2 and CDR3 according to SEQ ID NO:370, SEQ ID NO:371, and SEQ ID NO:372, respectively, and/or (ii) the variable light chain (VL) comprises CDR1, CDR2 and CDR3 according to SEQ ID NO:418, SEQ ID NO:419, SEQ ID NO:420, respectively;
(c) (i) the variable heavy chain (VH) comprises CDR1, CDR2 and CDR3 according to SEQ ID NO:373, SEQ ID NO:374, and SEQ ID NO:375 respectively, and/or (ii) the variable light chain (VL) comprises CDR1, CDR2 and CDR3 according to SEQ ID NO:421, SEQ ID NO:422, SEQ ID NO:423, respectively;
(d) (i) the variable heavy chain (VH) comprises CDR1, CDR2 and CDR3 according to SEQ ID NO:376, SEQ ID NO:377, and SEQ ID NO:378, respectively, and/or (ii) the variable light chain (VL) comprises CDR1, CDR2 and CDR3 according to SEQ ID NO:424, SEQ ID NO:425, SEQ ID NO:426, respectively;
(e) (i) the variable heavy chain (VH) comprises CDR1, CDR2 and CDR3 according to SEQ ID NO:379, SEQ ID NO:380, and SEQ ID NO:381 respectively, and/or (ii) the variable light chain (VL) comprises CDR1, CDR2 and CDR3 according to SEQ ID NO:427, SEQ ID NO:428, SEQ ID NO:429, respectively;
(f) (i) the variable heavy chain (VH) comprises CDR1, CDR2 and CDR3 according to SEQ ID NO:382, SEQ ID NO:383, and SEQ ID NO:384, respectively, and/or (ii) the variable light chain (VL) comprises CDR1, CDR2 and CDR3 according to SEQ ID NO:430, SEQ ID NO:431, SEQ ID NO:432, respectively;
(g) (i) the variable heavy chain (VH) comprises CDR1, CDR2 and CDR3 according to SEQ ID NO:385, SEQ ID NO:386, and SEQ ID NO:387, respectively, and/or (ii) the variable light chain (VL) comprises CDR1, CDR2 and CDR3 according to SEQ ID NO:433, SEQ ID NO:434, SEQ ID NO:435, respectively;
(h) (i) the variable heavy chain (VH) comprises CDR1, CDR2 and CDR3 according to SEQ ID NO:388, SEQ ID NO:389, and SEQ ID NO:390, respectively, and/or (ii) the variable light chain (VL) comprises CDR1, CDR2 and CDR3 according to SEQ ID NO:436, SEQ ID NO:437, SEQ ID NO:438, respectively;
(i) (i) the variable heavy chain (VH) comprises CDR1, CDR2 and CDR3 according to SEQ ID NO:391, SEQ ID NO:392, and SEQ ID NO:393 respectively, and/or
(ii) the variable light chain (VL) comprises CDR1, CDR2 and CDR3 according to SEQ ID NO:439, SEQ ID NO:440, SEQ ID NO:441, respectively;
(j) (i) the variable heavy chain (VH) comprises CDR1, CDR2 and CDR3 according to SEQ ID NO:394, SEQ ID NO:395, and SEQ ID NO:396, respectively, and/or (ii) the variable light chain (VL) comprises CDR1, CDR2 and CDR3 according to SEQ ID NO:442, SEQ ID NO:443, SEQ ID NO:444, respectively;
(k) (i) the variable heavy chain (VH) comprises CDR1, CDR2 and CDR3 according to SEQ ID NO:397, SEQ ID NO:398, and SEQ ID NO:399, respectively, and/or (ii) the variable light chain (VL) comprises CDR1, CDR2 and CDR3 according to SEQ ID NO:445, SEQ ID NO:446, SEQ ID NO:447, respectively;
(l) (i) the variable heavy chain (VH) comprises CDR1, CDR2 and CDR3 according to SEQ ID NO:400, SEQ ID NO:401, and SEQ ID NO:402, respectively, and/or (ii) the variable light chain (VL) comprises CDR1, CDR2 and CDR3 according to SEQ ID NO:448, SEQ ID NO:449, SEQ ID NO:450, respectively;
(m) (i) the variable heavy chain (VH) comprises CDR1, CDR2 and CDR3 according to SEQ ID NO:403, SEQ ID NO:404, and SEQ ID NO:405 respectively, and/or (ii) the variable light chain (VL) comprises CDR1, CDR2 and CDR3 according to SEQ ID NO:451, SEQ ID NO:452, SEQ ID NO:453, respectively;
(n) (i) the variable heavy chain (VH) comprises CDR1, CDR2 and CDR3 according to SEQ ID NO:406, SEQ ID NO:407, and SEQ ID NO:408, respectively, and/or (ii) the variable light chain (VL) comprises CDR1, CDR2 and CDR3 according to SEQ ID NO:454, SEQ ID NO:455, SEQ ID NO:456, respectively;
(o) (i) the variable heavy chain (VH) comprises CDR1, CDR2 and CDR3 according to SEQ ID NO:409, SEQ ID NO:410, and SEQ ID NO:411, respectively, and/or (ii) the variable light chain (VL) comprises CDR1, CDR2 and CDR3 according to SEQ ID NO:457, SEQ ID NO:458, SEQ ID NO:459, respectively;
(p) (i) the variable heavy chain (VH) comprises CDR1, CDR2 and CDR3 according to SEQ ID NO:412, SEQ ID NO:413, and SEQ ID NO:414, respectively, and/or (ii) the variable light chain (VL) comprises CDR1, CDR2 and CDR3 according to SEQ ID NO:460, SEQ ID NO:461, SEQ ID NO:462, respectively;
(q) (i) the variable heavy chain (VH) comprises CDR1, CDR2 and CDR3 according to SEQ ID NO:415, SEQ ID NO:416, and SEQ ID NO:417, respectively, and/or (ii) the variable light chain (VL) comprises CDR1, CDR2 and CDR3 according to SEQ ID NO:463, SEQ ID NO:464, SEQ ID NO:465, respectively;
(r) (i) the variable heavy chain (VH) comprises CDR1, CDR2 and CDR3 according to SEQ ID NO:584, SEQ ID NO:585, and SEQ ID NO:586, respectively, and/or (ii) the variable light chain (VL) comprises CDR1, CDR2 and CDR3 according to SEQ ID NO:596, SEQ ID NO:597, SEQ ID NO:598, respectively;
(s) (i) the variable heavy chain (VH) comprises CDR1, CDR2 and CDR3 according to SEQ ID NO:587, SEQ ID NO:588, and SEQ ID NO:589, respectively, and/or (ii) the variable light chain (VL) comprises CDR1, CDR2 and CDR3 according to SEQ ID NO:599, SEQ ID NO:601, SEQ ID NO:601, respectively;
(t) (i) the variable heavy chain (VH) comprises CDR1, CDR2 and CDR3 according to SEQ ID NO:590, SEQ ID NO:591, and SEQ ID NO:592, respectively, and/or (ii) the variable light chain (VL) comprises CDR1, CDR2 and CDR3 according to SEQ ID NO:602, SEQ ID NO:603, SEQ ID NO:604, respectively; or
(u) (i) the variable heavy chain (VH) comprises CDR1, CDR2 and CDR3 according to SEQ ID NO:593, SEQ ID NO:594, and SEQ ID NO:595, respectively, and/or (ii) the variable light chain (VL) comprises CDR1, CDR2 and CDR3 according to SEQ ID NO:605, SEQ ID NO:606, SEQ ID NO:607, respectively, or SEQ ID NO: 608, SEQ ID NO: 609, SEQ ID NO: 610, respectively.

70. The method of claim 68 or 69, wherein the variable heavy chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NOs: 45-131 and 534-544.

71. The method of claim 68 or 69, wherein the variable light chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NO: 132-227 and 545-558.

72. The method of any one of claim 68-71, wherein the TCR Vβ region is selected from the group consisting of Vβ1, Vβ2, Vβ3, Vβ4, Vβ5.1, Vβ5.3, Vβ7.1, Vβ7.2, Vβ8, Vβ9, Vβ11, Vβ12, Vβ13.1, Vβ13.2, Vβ13.6, Vβ14, Vβ16, Vβ17, Vβ18, Vβ20, and Vβ22.

73. The method of claim 72, wherein the TCR Vβ region is Vβ2.

74. The method of any one of claim 68 or 69, wherein the T-cell mediated disease is selected from the group consisting of Atherosclerosis, Parkinson's disease, Alzheimer's disease, Type 1 Diabetes Mellitus, Grave's Disease, Hashimoto's Thyroiditis, Addison's Disease, Rheumatoid Arthritis (RA), Multiple Sclerosis (MS), Psoriasis, Psoriatic Arthritis, Lichen Planus, Lichen Planopilaris, Morphea, Scleroderma, Systemic Sclerosis, acute or chronic Graft-Versus-Host Disease (GVHD), Cardiac Organ Transplant Rejection, Pulmonary Organ Transplant Rejection, Renal Organ Transplant Rejection, Interstitial Lung Disease, Scleroderma, Alopecia Areata, Vitiligo, Celiac Disease (Sprue), Myasthenia Gravis, Pernicious Anemia, Sjogren's Syndrome, Systemic Lupus Erythematosus, Chronic Inflammatory Demyelinating Polyneuropathy, Guillain-Barre Syndrome, Inflammatory Bowel Disease, Crohn's Disease, and Ulcerative Colitis.

75. The method of any one of claim 68-74, wherein the composition is delivered intratumorally, intravenously, intraperitoneally, intramuscularly, intrathecally, transdermally, or subcutaneously.

76. The method of claim 75, wherein the composition is delivered intravenously.

77. The method of any one of claim 68-76, wherein the subject is a human.

78. A method for treating T-cell mediated diseases in a subject in need thereof, the method comprising: administering to the subject a therapeutically effective amount of a composition comprising an antibody or antigen-binding fragment thereof, capable of specifically binding to a TCR Vβ region, comprising a (i) heavy chain variable region (VH) having an amino acid sequence according to any of SEQ ID NOs: 228-295 and 559-569, and/or
(ii) a light chain variable region (VL) having an amino acid sequence according to any of SEQ ID NOs: 296-369 and 570-583.

79. The method of claim 78, wherein the antibody or antigen-binding fragment comprises
(i) a variable heavy chain (VH) which comprises:
(a) CDR1, CDR2 and CDR3 according to SEQ ID NO:10, SEQ ID NO:11, and SEQ ID NO:12, respectively;
(b) CDR1, CDR2 and CDR3 according to SEQ ID NO:370, SEQ ID NO:371, and SEQ ID NO:372, respectively;
(c) CDR1, CDR2 and CDR3 according to SEQ ID NO:373, SEQ ID NO:374, and SEQ ID NO:375 respectively;
(d) CDR1, CDR2 and CDR3 according to SEQ ID NO:376, SEQ ID NO:377, and SEQ ID NO:378, respectively;
(e) CDR1, CDR2 and CDR3 according to SEQ ID NO:379, SEQ ID NO:380, and SEQ ID NO:381 respectively;
(f) CDR1, CDR2 and CDR3 according to SEQ ID NO:382, SEQ ID NO:383, and SEQ ID NO:384, respectively;
(g) CDR1, CDR2 and CDR3 according to SEQ ID NO:385, SEQ ID NO:386, and SEQ ID NO:387, respectively;
(h) CDR1, CDR2 and CDR3 according to SEQ ID NO:388, SEQ ID NO:389, and SEQ ID NO:390, respectively;
(i) CDR1, CDR2 and CDR3 according to SEQ ID NO:391, SEQ ID NO:392, and SEQ ID NO:393, respectively;
(j) CDR1, CDR2 and CDR3 according to SEQ ID NO:394, SEQ ID NO:395, and SEQ ID NO:396, respectively;
(k) CDR1, CDR2 and CDR3 according to SEQ ID NO:397, SEQ ID NO:398, and SEQ ID NO:399, respectively;
(l) CDR1, CDR2 and CDR3 according to SEQ ID NO:400, SEQ ID NO:401, and SEQ ID NO:402, respectively;
(m) CDR1, CDR2 and CDR3 according to SEQ ID NO:403, SEQ ID NO:404, and SEQ ID NO:405, respectively;
(n) CDR1, CDR2 and CDR3 according to SEQ ID NO:406, SEQ ID NO:407, and SEQ ID NO:408, respectively;
(o) CDR1, CDR2 and CDR3 according to SEQ ID NO:409, SEQ ID NO:410, and SEQ ID NO:411, respectively;
(p) CDR1, CDR2 and CDR3 according to SEQ ID NO:412, SEQ ID NO:413, and SEQ ID NO:414, respectively;
(q) CDR1, CDR2 and CDR3 according to SEQ ID NO:415, SEQ ID NO:416, and SEQ ID NO:417, respectively;
(r) CDR1, CDR2 and CDR3 according to SEQ ID NO:584, SEQ ID NO:585, and SEQ ID NO:586, respectively;
(s) CDR1, CDR2 and CDR3 according to SEQ ID NO:587, SEQ ID NO:588, and SEQ ID NO:589, respectively;
(t) CDR1, CDR2 and CDR3 according to SEQ ID NO:590, SEQ ID NO:591, and SEQ ID NO:592, respectively; or
(u) CDR1, CDR2 and CDR3 according to SEQ ID NO:593, SEQ ID NO:594, and SEQ ID NO:595, respectively; and/or
(ii) a variable light chain (VL) which comprises:
(a) CDR1, CDR2 and CDR3 according to SEQ ID NO:13, SEQ ID NO:14, SEQ ID NO:15,
(b) CDR1, CDR2 and CDR3 according to SEQ ID NO:418, SEQ ID NO:419, SEQ ID NO:420, respectively;
(c) CDR1, CDR2 and CDR3 according to SEQ ID NO:421, SEQ ID NO:422, SEQ ID NO:423, respectively;
(d) CDR1, CDR2 and CDR3 according to SEQ ID NO:424, SEQ ID NO:425, SEQ ID NO:426, respectively;
(e) CDR1, CDR2 and CDR3 according to SEQ ID NO:427, SEQ ID NO:428, SEQ ID NO:429, respectively;
(f) CDR1, CDR2 and CDR3 according to SEQ ID NO:430, SEQ ID NO:431, SEQ ID NO:432, respectively;
(g) CDR1, CDR2 and CDR3 according to SEQ ID NO:433, SEQ ID NO:434, SEQ ID NO:435, respectively;
(h) CDR1, CDR2 and CDR3 according to SEQ ID NO:436, SEQ ID NO:437, SEQ ID NO:438, respectively;
(i) CDR1, CDR2 and CDR3 according to SEQ ID NO:439, SEQ ID NO:440, SEQ ID NO:441, respectively;
(j) CDR1, CDR2 and CDR3 according to SEQ ID NO:442, SEQ ID NO:443, SEQ ID NO:444, respectively;
(k) CDR1, CDR2 and CDR3 according to SEQ ID NO:445, SEQ ID NO:446, SEQ ID NO:447, respectively;
(l) CDR1, CDR2 and CDR3 according to SEQ ID NO:448, SEQ ID NO:449, SEQ ID NO:450, respectively;
(m) CDR1, CDR2 and CDR3 according to SEQ ID NO:451, SEQ ID NO:452, SEQ ID NO:453, respectively;
(n) CDR1, CDR2 and CDR3 according to SEQ ID NO:454, SEQ ID NO:455, SEQ ID NO:456, respectively;
(o) CDR1, CDR2 and CDR3 according to SEQ ID NO:457, SEQ ID NO:458, SEQ ID NO:459, respectively;
(p) CDR1, CDR2 and CDR3 according to SEQ ID NO:460, SEQ ID NO:461, SEQ ID NO:462, respectively;
(q) CDR1, CDR2 and CDR3 according to SEQ ID NO:463, SEQ ID NO:464, SEQ ID NO:465, respectively;
(r) CDR1, CDR2 and CDR3 according to SEQ ID NO:596, SEQ ID NO:597, SEQ ID NO:598, respectively;
(s) CDR1, CDR2 and CDR3 according to SEQ ID NO:599, SEQ ID NO:601, SEQ ID NO:601, respectively;
(t) CDR1, CDR2 and CDR3 according to SEQ ID NO:602, SEQ ID NO:603, SEQ ID NO:604, respectively; or
(u) CDR1, CDR2 and CDR3 according to SEQ ID NO:605, SEQ ID NO:606, SEQ ID NO:607, respectively, or SEQ ID NO: 608, SEQ ID NO: 609, SEQ ID NO: 610, respectively.

80. The method of claim 78 or 79, wherein the variable heavy chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NOs: 228-295 and 559-569.

81. The method of claim 78 or 79, wherein the variable light chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NO: 296-369 and 570-583.

82. The antibody or antigen-binding fragment of claim 78, wherein the antibody or antigen-binding fragment further comprises a light chain variable region (VL) having an amino acid sequence according to any of SEQ ID NOs: 296-369 and 570-583.

83. The antibody or antigen-binding fragment of claim 78, wherein the antibody or antigen-binding fragment further comprises a heavy chain variable region (VH) having an amino acid sequence according to any of SEQ ID NOs: 228-295 and 559-569.

84. The method of any one of claim 78-83, wherein the TCR Vβ region is selected from the group consisting of Vβ1, Vβ2, Vβ3, Vβ4, Vβ5.1, Vβ5.3, Vβ7.1, Vβ7.2, Vβ8, Vβ9, Vβ11, Vβ12, Vβ13.1, Vβ13.2, Vβ13.6, Vβ14, Vβ16, Vβ17, Vβ18, Vβ20, and Vβ22.

85. The method of claim 84, wherein the TCR Vβ region is Vβ2.

86. The method of any one of claim 78-85, wherein the T-cell mediated disease is selected from the group consisting of Atherosclerosis, Parkinson's disease, Alzheimer's disease, Type 1 Diabetes Mellitus, Grave's Disease, Hashimoto's Thyroiditis, Addison's Disease, Rheumatoid Arthritis (RA), Multiple Sclerosis (MS), Psoriasis, Psoriatic Arthritis, Lichen Planus, Lichen Planopilaris, Morphea, Scleroderma, Systemic Sclerosis, acute or chronic Graft-Versus-Host Disease (GVHD), Cardiac Organ Transplant Rejection, Pulmonary Organ Transplant Rejection, Renal Organ Transplant Rejection, Interstitial Lung Disease, Scleroderma, Alopecia Areata, Vitiligo, Celiac Disease (Sprue), Myasthenia Gravis, Pernicious Anemia, Sjogren's Syndrome, Systemic Lupus Erythematosus, Chronic Inflammatory Demyelinating Polyneuropathy, Guillain-Barre Syndrome, Inflammatory Bowel Disease, Crohn's Disease, and Ulcerative Colitis.

87. The method of any one of claim 78-86, wherein the composition is delivered intratumorally, intravenously, intraperitoneally, intramuscularly, intrathecally or subcutaneous delivery.

88. The method of claim 87, wherein the composition is delivered intravenously.

89. The method of any one of claim 78-88, wherein the subject is a human.

90. Use of (a) the CAR of any one of claim 1-18, or (b) the antibody or antigen binding fragment of any one of claim 26-44, for the manufacture of a medicament for treating a T-cell mediated disease.

91. The use of claim 90, wherein the T-cell mediated disease is selected from the group consisting of Atherosclerosis, Parkinson's disease, Alzheimer's disease, Type 1 Diabetes Mellitus, Grave's Disease, Hashimoto's Thyroiditis, Addison's Disease, Rheumatoid Arthritis (RA), Multiple Sclerosis (MS), Psoriasis, Psoriatic Arthritis, Lichen Planus, Lichen Planopilaris, Morphea, Scleroderma, Systemic Sclerosis, acute or chronic Graft-Versus-Host Disease (GVHD), Cardiac Organ Transplant Rejection, Pulmonary Organ Transplant Rejection, Renal Organ Transplant Rejection, Interstitial Lung Disease, Scleroderma, Alopecia Areata, Vitiligo, Celiac Disease (Sprue), Myasthenia Gravis, Pernicious Anemia, Sjogren's Syndrome, Systemic Lupus Erythematosus, Chronic Inflammatory Demyelinating Polyneuropathy, Guillain-Barre Syndrome, Inflammatory Bowel Disease, Crohn's Disease, and Ulcerative Colitis.

92. Use of (a) the CAR of any one of claim 1-18, or (b) the antibody or antigen binding fragment of any one of claim 26-44, for the manufacture of a medicament for treating a cancer.

93. The use of claim 92, wherein the wherein the cancer is selected from the group consisting of T-cell Lymphoma, T-cell Leukemia, Cutaneous T-cell Lymphoma, Peripheral T-cell Lymphoma (PTCL), Not Otherwise Specified PTCL (PTCL-NOS), Angioimmunoblastic T-cell Lymphoma (AITL), Anaplastic Large-cell Lymphoma (ALCL), Enteropathy-Associated T-cell Lymphoma (EATL), Adult T-cell Leukemia/Lymphoma (ATLL), Hepatosplenic T-cell Lymphoma (HSTL), Subcutaneous Panniculitis-Like T-cell Lymphoma (SPTCL), T-cell Acute Lymphoblastic Leukemia (T-ALL), T-cell Chronic Lymphocytic Leukemia, Large Granular Lymphocyte Leukemia, T-cell Prolymphocytic Leukemia, Lympomatoid Papulosis, Small Medium Pleiomorphic T-cell Lymphoma, Mycosis Fungoides, Sezary Syndrome, and Cytotoxic T-cell Lymphoma.

94. A kit of parts comprising at least one of a (i) variable heavy chain, and/or (ii) variable light chain of the (a) the CAR of any one of claim 1-18, or
(b) the antibody or antigen binding fragment of any one of claim 26-44.

95. The kit of claim 94, wherein the kit further comprises instructions for use.

96. An adenoassociated virus (AAV) vector encoding a chimeric antigen receptor (CAR) comprising
(i) an antigen binding molecule that specifically binds to a TCR Vβ region, (ii) an extracellular domain, (iii) a transmembrane domain, (iv) a costimulatory domain and (v) an activating domain,
wherein the antigen binding molecule is a single chain variable fragment (scFv) comprising:
(a) a variable heavy chain according to any one of SEQ ID NOs: 45-131 and 534-544, and/or
(b) a variable light chain according to any one of SEQ ID NOs: 132-227 and 545-558.

97. The AAV vector of claim 96, wherein
(i) a variable heavy chain (VH) which comprises:
(a) CDR1, CDR2 and CDR3 according to SEQ ID NO:10, SEQ ID NO:11, and SEQ ID NO:12, respectively;
(b) CDR1, CDR2 and CDR3 according to SEQ ID NO:370, SEQ ID NO:371, and SEQ ID NO:372, respectively;
(c) CDR1, CDR2 and CDR3 according to SEQ ID NO:373, SEQ ID NO:374, and SEQ ID NO:375 respectively;
(d) CDR1, CDR2 and CDR3 according to SEQ ID NO:376, SEQ ID NO:377, and SEQ ID NO:378, respectively;
(e) CDR1, CDR2 and CDR3 according to SEQ ID NO:379, SEQ ID NO:380, and SEQ ID NO:381 respectively;
(f) CDR1, CDR2 and CDR3 according to SEQ ID NO:382, SEQ ID NO:383, and SEQ ID NO:384, respectively;
(g) CDR1, CDR2 and CDR3 according to SEQ ID NO:385, SEQ ID NO:386, and SEQ ID NO:387, respectively;
(h) CDR1, CDR2 and CDR3 according to SEQ ID NO:388, SEQ ID NO:389, and SEQ ID NO:390, respectively;
(i) CDR1, CDR2 and CDR3 according to SEQ ID NO:391, SEQ ID NO:392, and SEQ ID NO:393, respectively;
(j) CDR1, CDR2 and CDR3 according to SEQ ID NO:394, SEQ ID NO:395, and SEQ ID NO:396, respectively;
(k) CDR1, CDR2 and CDR3 according to SEQ ID NO:397, SEQ ID NO:398, and SEQ ID NO:399, respectively;
(l) CDR1, CDR2 and CDR3 according to SEQ ID NO:400, SEQ ID NO:401, and SEQ ID NO:402, respectively;
(m) CDR1, CDR2 and CDR3 according to SEQ ID NO:403, SEQ ID NO:404, and SEQ ID NO:405, respectively;
(n) CDR1, CDR2 and CDR3 according to SEQ ID NO:406, SEQ ID NO:407, and SEQ ID NO:408, respectively;
(o) CDR1, CDR2 and CDR3 according to SEQ ID NO:409, SEQ ID NO:410, and SEQ ID NO:411, respectively;
(p) CDR1, CDR2 and CDR3 according to SEQ ID NO:412, SEQ ID NO:413, and SEQ ID NO:414, respectively;
(q) CDR1, CDR2 and CDR3 according to SEQ ID NO:415, SEQ ID NO:416, and SEQ ID NO:417, respectively;
(r) CDR1, CDR2 and CDR3 according to SEQ ID NO:584, SEQ ID NO:585, and SEQ ID NO:586, respectively;
(s) CDR1, CDR2 and CDR3 according to SEQ ID NO:587, SEQ ID NO:588, and SEQ ID NO:589, respectively;
(t) CDR1, CDR2 and CDR3 according to SEQ ID NO:590, SEQ ID NO:591, and SEQ ID NO:592, respectively; or
(u) CDR1, CDR2 and CDR3 according to SEQ ID NO:593, SEQ ID NO:594, and SEQ ID NO:595, respectively; and/or
(ii) a variable light chain (VL) which comprises:
(a) CDR1, CDR2 and CDR3 according to SEQ ID NO:13, SEQ ID NO:14, SEQ ID NO:15,
(b) CDR1, CDR2 and CDR3 according to SEQ ID NO:418, SEQ ID NO:419, SEQ ID NO:420, respectively;
(c) CDR1, CDR2 and CDR3 according to SEQ ID NO:421, SEQ ID NO:422, SEQ ID NO:423, respectively;
(d) CDR1, CDR2 and CDR3 according to SEQ ID NO:424, SEQ ID NO:425, SEQ ID NO:426, respectively;
(e) CDR1, CDR2 and CDR3 according to SEQ ID NO:427, SEQ ID NO:428, SEQ ID NO:429, respectively;
(f) CDR1, CDR2 and CDR3 according to SEQ ID NO:430, SEQ ID NO:431, SEQ ID NO:432, respectively;
(g) CDR1, CDR2 and CDR3 according to SEQ ID NO:433, SEQ ID NO:434, SEQ ID NO:435, respectively;
(h) CDR1, CDR2 and CDR3 according to SEQ ID NO:436, SEQ ID NO:437, SEQ ID NO:438, respectively;
(i) CDR1, CDR2 and CDR3 according to SEQ ID NO:439, SEQ ID NO:440, SEQ ID NO:441, respectively;
(j) CDR1, CDR2 and CDR3 according to SEQ ID NO:442, SEQ ID NO:443, SEQ ID NO:444, respectively;
(k) CDR1, CDR2 and CDR3 according to SEQ ID NO:445, SEQ ID NO:446, SEQ ID NO:447, respectively;
(l) CDR1, CDR2 and CDR3 according to SEQ ID NO:448, SEQ ID NO:449, SEQ ID NO:450, respectively;
(m) CDR1, CDR2 and CDR3 according to SEQ ID NO:451, SEQ ID NO:452, SEQ ID NO:453, respectively;
(n) CDR1, CDR2 and CDR3 according to SEQ ID NO:454, SEQ ID NO:455, SEQ ID NO:456, respectively;
(o) CDR1, CDR2 and CDR3 according to SEQ ID NO:457, SEQ ID NO:458, SEQ ID NO:459, respectively;
(p) CDR1, CDR2 and CDR3 according to SEQ ID NO:460, SEQ ID NO:461, SEQ ID NO:462, respectively;
(q) CDR1, CDR2 and CDR3 according to SEQ ID NO:463, SEQ ID NO:464, SEQ ID NO:465, respectively;
(r) CDR1, CDR2 and CDR3 according to SEQ ID NO:596, SEQ ID NO:597, SEQ ID NO:598, respectively;
(s) CDR1, CDR2 and CDR3 according to SEQ ID NO:599, SEQ ID NO:601, SEQ ID NO:601, respectively;
(t) CDR1, CDR2 and CDR3 according to SEQ ID NO:602, SEQ ID NO:603, SEQ ID NO:604, respectively; or
(u) CDR1, CDR2 and CDR3 according to SEQ ID NO:605, SEQ ID NO:606, SEQ ID NO:607, respectively, or SEQ ID NO: 608, SEQ ID NO: 609, SEQ ID NO: 610, respectively.

98. The AAV vector of claim 96 or 97, wherein the variable heavy chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NOs: 45-131 and 534-544.

99. The AAV vector of claim 96 or 97, wherein the variable light chain comprises an amino acid sequence having at least 85%, at least 90%, at least 91% at least 92%, at least 93%, at least 94%, at least 95%, at least 96%, at least 97%, at least 98%, at least 99%, or about 100% sequence identity to any one of SEQ ID NO: 132-227 and 545-558.

100. The AAV vector of any one of claim 96-99, wherein the TCR Vβ region is selected from the group consisting of Vβ1, Vβ2, Vβ3, Vβ4, Vβ5.1, Vβ5.3, Vβ7.1, Vβ7.2, Vβ8, Vβ9, Vβ11, Vβ12, Vβ13.1, Vβ13.2, Vβ13.6, Vβ14, Vβ16, Vβ17, Vβ18, Vβ20, and Vβ22.

101. The AAV vector of claim 100, wherein the TCR Vβ region is Vβ2.

102. The AAV vector of claim 96, wherein the antigen-binding domain is a scFv.

103. The AAV vector of claim 102, wherein the scFv comprises an amino acid sequence according to any of SEQ ID NOs: 14-44.

104. The AAV vector of any one of claim 96-103, wherein the costimulatory domain comprises a 4-1BB costimulatory domain.

105. The AAV vector of claim 104, wherein the 4-1BB costimulatory domain comprises an amino acid sequence according to SEQ ID NO: 8.

106. The AAV vector of any one of claim 96-105, wherein the transmembrane domain comprises an hCD28 transmembrane.

107. The AAV vector of claim 106, wherein the transmembrane domain comprises a hCD28 amino acid sequence according to SEQ ID NO: 7.

108. The AAV vector of any one of claim 96-107, wherein the signaling domain comprises a CD247 signaling domain.

109. The AAV vector of claim 108, wherein the signaling domain comprises a CD247 signaling domain amino acid sequence according to SEQ ID NO: 9.

110. The AAV vector of any one of claim 96-109, wherein the scFv comprises a CD28 signal peptide.

111. The AAV vector of claim 110, wherein the scFv comprises a CD28 signal peptide an amino acid sequence according to SEQ ID NO:3.

112. The AAV vector of any one of claim 96-111, wherein the scFv comprises a linker peptide.

113. The AAV vector of claim 112, wherein the scFv comprises a linker peptide according to SEQ ID NO: 4.

114. The method of any of claim 45-89, further comprising determining the subject's TCR Vβ usage.

115. The method of claim 114, wherein determining the subject's TCR Vβ usage comprises obtaining a sample from the subject, wherein the sample is a blood sample or a tissue sample.

116. The method of claim 114 or 115, wherein the subject is administered (a) a CAR of any one of claim 1-18, or (b) the antibody or antigen binding fragment of any one of claim 26-44, that corresponds to the subject's TCR Vβ usage.

117. The antibody or antigen-binding fragment of claim 26 or 27, wherein the antibody or antigen-binding fragment comprises (i) a variable heavy chain (VH) which comprises:
(a) CDR1, CDR2 and CDR3 according to SEQ ID NO:10, SEQ ID NO:11, and SEQ ID NO:12, respectively;
(b) CDR1, CDR2 and CDR3 according to SEQ ID NO:370, SEQ ID NO:371, and SEQ ID NO:372, respectively;
(c) CDR1, CDR2 and CDR3 according to SEQ ID NO:373, SEQ ID NO:374, and SEQ ID NO:375 respectively;
(d) CDR1, CDR2 and CDR3 according to SEQ ID NO:376, SEQ ID NO:377, and SEQ ID NO:378, respectively;
(e) CDR1, CDR2 and CDR3 according to SEQ ID NO:379, SEQ ID NO:380, and SEQ ID NO:381 respectively;
(f) CDR1, CDR2 and CDR3 according to SEQ ID NO:382, SEQ ID NO:383, and SEQ ID NO:384, respectively;
(g) CDR1, CDR2 and CDR3 according to SEQ ID NO:385, SEQ ID NO:386, and SEQ ID NO:387, respectively;
(h) CDR1, CDR2 and CDR3 according to SEQ ID NO:388, SEQ ID NO:389, and SEQ ID NO:390, respectively;
(i) CDR1, CDR2 and CDR3 according to SEQ ID NO:391, SEQ ID NO:392, and SEQ ID NO:393, respectively;
(j) CDR1, CDR2 and CDR3 according to SEQ ID NO:394, SEQ ID NO:395, and SEQ ID NO:396, respectively;
(k) CDR1, CDR2 and CDR3 according to SEQ ID NO:397, SEQ ID NO:398, and SEQ ID NO:399, respectively;
(l) CDR1, CDR2 and CDR3 according to SEQ ID NO:400, SEQ ID NO:401, and SEQ ID NO:402, respectively;
(m) CDR1, CDR2 and CDR3 according to SEQ ID NO:403, SEQ ID NO:404, and SEQ ID NO:405, respectively;
(n) CDR1, CDR2 and CDR3 according to SEQ ID NO:406, SEQ ID NO:407, and SEQ ID NO:408, respectively;
(o) CDR1, CDR2 and CDR3 according to SEQ ID NO:409, SEQ ID NO:410, and SEQ ID NO:411, respectively;
(p) CDR1, CDR2 and CDR3 according to SEQ ID NO:412, SEQ ID NO:413, and SEQ ID NO:414, respectively;
(q) CDR1, CDR2 and CDR3 according to SEQ ID NO:415, SEQ ID NO:416, and SEQ ID NO:417, respectively;
(r) CDR1, CDR2 and CDR3 according to SEQ ID NO:584, SEQ ID NO:585, and SEQ ID NO:586, respectively;
(s) CDR1, CDR2 and CDR3 according to SEQ ID NO:587, SEQ ID NO:588, and SEQ ID NO:589, respectively;
(t) CDR1, CDR2 and CDR3 according to SEQ ID NO:590, SEQ ID NO:591, and SEQ ID NO:592, respectively; or
(u) CDR1, CDR2 and CDR3 according to SEQ ID NO:593, SEQ ID NO:594, and SEQ ID NO:595, respectively; and/or
(ii) a variable light chain (VL) which comprises:
(a) CDR1, CDR2 and CDR3 according to SEQ ID NO:13, SEQ ID NO:14, SEQ ID NO:15, respectively;
(b) CDR1, CDR2 and CDR3 according to SEQ ID NO:418, SEQ ID NO:419, SEQ ID NO:420, respectively;
(c) CDR1, CDR2 and CDR3 according to SEQ ID NO:421, SEQ ID NO:422, SEQ ID NO:423, respectively;
(d) CDR1, CDR2 and CDR3 according to SEQ ID NO:424, SEQ ID NO:425, SEQ ID NO:426, respectively;
(e) CDR1, CDR2 and CDR3 according to SEQ ID NO:427, SEQ ID NO:428, SEQ ID NO:429, respectively;
(f) CDR1, CDR2 and CDR3 according to SEQ ID NO:430, SEQ ID NO:431, SEQ ID NO:432, respectively;
(g) CDR1, CDR2 and CDR3 according to SEQ ID NO:433, SEQ ID NO:434, SEQ ID NO:435, respectively;
(h) CDR1, CDR2 and CDR3 according to SEQ ID NO:436, SEQ ID NO:437, SEQ ID NO:438, respectively;
(i) CDR1, CDR2 and CDR3 according to SEQ ID NO:439, SEQ ID NO:440, SEQ ID NO:441, respectively;
(j) CDR1, CDR2 and CDR3 according to SEQ ID NO:442, SEQ ID NO:443, SEQ ID NO:444, respectively;
(k) CDR1, CDR2 and CDR3 according to SEQ ID NO:445, SEQ ID NO:446, SEQ ID NO:447, respectively;
(l) CDR1, CDR2 and CDR3 according to SEQ ID NO:448, SEQ ID NO:449, SEQ ID NO:450, respectively;
(m) CDR1, CDR2 and CDR3 according to SEQ ID NO:451, SEQ ID NO:452, SEQ ID NO:453, respectively;
(n) CDR1, CDR2 and CDR3 according to SEQ ID NO:454, SEQ ID NO:455, SEQ ID NO:456, respectively;
(o) CDR1, CDR2 and CDR3 according to SEQ ID NO:457, SEQ ID NO:458, SEQ ID NO:459, respectively;
(p) CDR1, CDR2 and CDR3 according to SEQ ID NO:460, SEQ ID NO:461, SEQ ID NO:462, respectively;
(q) CDR1, CDR2 and CDR3 according to SEQ ID NO:463, SEQ ID NO:464, SEQ ID NO:465, respectively;
(r) CDR1, CDR2 and CDR3 according to SEQ ID NO:596, SEQ ID NO:597, SEQ ID NO:598, respectively;
(s) CDR1, CDR2 and CDR3 according to SEQ ID NO:599, SEQ ID NO:601, SEQ ID NO:601, respectively;
(t) CDR1, CDR2 and CDR3 according to SEQ ID NO:602, SEQ ID NO:603, SEQ ID NO:604, respectively; or
(u) CDR1, CDR2 and CDR3 according to SEQ ID NO:605, SEQ ID NO:606, SEQ ID NO:607, respectively, or SEQ ID NO: 608, SEQ ID NO: 609, SEQ ID NO: 610, respectively.

118. The antibody or antigen-binding fragment of any one of claim 26, 27, 56, or 78, wherein the ADCC activity of the antibody composition is increased or decreased by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 1-fold, about 2-fold, about 3-fold, or about 4-fold, or increased or decreased by about 5% to about 400%.

119. The antibody or antigen-binding fragment of claim 118, wherein the antibody or antigen binding fragment has an increased antibody-dependent cell mediated cytotoxicity activity achieved through afucosylation.

120. The antibody or antigen-binding fragment of claim 119, wherein the antibody or antigen binding fragment has an increased antibody-dependent cell mediated cytotoxicity activity achieved through glycosylation.


SEQUENCE LISTING (631 sequences)

SEQ ID NO: 1 - DNA, 4180 bp, synthetic construct
  misc_feature 1..4180: note = AAV Nucleotide Sequence
  source 1..4180: mol_type = other DNA
tctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatgagggcagaggcagtctgctgacatgcggtgacgtggaagagaatcccggccctatgctcaggctgctcttggctctcaacttattcccttcaattcaagtaacaggagaagtccaacttcaacagagtgtcgcggatcttgttcggccaggggcctcacttaaattgtcatgtacggcttctggctttaacataaagtccgcttacatgcactgggtgatacaacggcctgaccaatgtcccgagtgtcttggcagaattgatccagccaccgggaaaactaagtacgcgccgaagtttcaagcaaaagcgaccataaccgcagacacttcctctaacacagcctacttgcaactgagttcactcacgagtgaggataccgctatttattattgcacgcgaagtttgaattgggattacggtttggattactggggtcagggcacttccgttaccgtttcctccggctccacctctggatccggcaagcccggatctggcgagggatccaccaagggcgacatcgtattgacccagtcaccggcaagtttggctgtgtcacttgggcaaagagccacaatcagttgccgggcatcaaaatccgttagtattctgggaacacatctcatccattggtatcaacagaagccgggccaacctccgaaattgctgatttatgctgccagcaatctcgaatctggagtccccgcgcggttcagcgggagtgggtctgagacagtgtttaccttgaacattcatcctgtagaagaggaagacgctgcgacatatttctgtcaacagtctattgaagacccttggacgtttggttgcggcaccaagttgggcatcaaaggtaccaccacaacgcccgctcctcggccaccgacgccagcgccaactattgcgagtcagcctctcagtctgcgacctgaggcttgtcgaccagcagccggaggcgcagtgcacacgagggggctggacttcgcctgtgatagaagacctccttctaagcccttttgggtgctggtggtggttggtggagtcctggcttgctatagcttgctagtaacagtggcctttattattttctgggtgaggaaacggggcagaaagaaactcctgtatatattcaaacaaccatttatgagaccagtacaaactactcaagaggaagatggctgtagctgccgatttccagaagaagaagaaggaggatgtgaactggctagcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctagcgccacgaacttctctctgttaaagcaagcaggcgacgtggaagaaaaccccggtcccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcc

SEQ ID NO: 2 - protein, 330 aa, synthetic construct
  REGION 1..330: note = FC sequence with ADCC enhancing mutations
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSFEDPEVKFNWYVDGVEVHNAKTKPREEQYNSTYRVVSVLTVLHQDWLNGKEYKCKVTNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 18 aa, synthetic construct
  REGION 1..18: note = CD28 signal peptide
MLRLLLALNLFPSIQVTG

SEQ ID NO: 4 - protein, 18 aa, synthetic construct
  REGION 1..18: note = Linker
GSTSGSGKPGSGEGSTKG

SEQ ID NO: 5 - protein, 242 aa, synthetic construct
  REGION 1..242: note = CD8a hinge-CD28 transmembrane-4-1BB signal-CD247 signal
GTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 6 - protein, 50 aa, synthetic construct
  REGION 1..50: note = CD8a hinge
GTTTTPAPRPPTPAPTIASQPLSLRPEACRPAAGGAVHTRGLDFACDRRP

SEQ ID NO: 7 - protein, 32 aa, synthetic construct
  REGION 1..32: note = CD28 transmembrane
PSKPFWVLVVVGGVLACYSLLVTVAFIIFWVR

SEQ ID NO: 8 - protein, 44 aa, synthetic construct
  REGION 1..44: note = 4-1BB signal
KRGRKKLLYIFKQPFMRPVQTTQEEDGCSCRFPEEEEGGCELAS

SEQ ID NO: 9 - protein, 116 aa, synthetic construct
  REGION 1..116: note = CD247 signal
LRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 10 - protein, 7 aa, synthetic construct
  REGION 1..7: note = VB2 VH CDR1
GFNIKSA

SEQ ID NO: 11 - protein, 6 aa, synthetic construct
  REGION 1..6: note = VB2 VH CDR2
DPATGK

SEQ ID NO: 12 - protein, 10 aa, synthetic construct
  REGION 1..10: note = VB2 VH CDR3
SLNWDYGLDY

SEQ ID NO: 13 - protein, 15 aa, synthetic construct
  REGION 1..15: note = VB2 VL CDR1
RASKSVSILGTHLIH

SEQ ID NO: 14 - protein, 7 aa, synthetic construct
  REGION 1..7: note = VB2 VL CDR2
AASNLES

SEQ ID NO: 15 - protein, 9 aa, synthetic construct
  REGION 1..9: note = VB2 VL CDR3
QQSIEDPWT

SEQ ID NO: 16 - protein, 508 aa, synthetic construct
  REGION 1..508: note = mCAR-Vb2 Complete CAR
MLRLLLALNLFPSIQVTGEVQLQQSVADLVRPGASLKLSCTASGFNIKSAYMHWVIQRPDQCPECLGRIDPATGKTKYAPKFQAKATITADTSSNTAYLQLSSLTSEDTAIYYCTRSLNWDYGLDYWGQGTSVTVSSGSTSGSGKPGSGEGSTKGDIVLTQSPASLAVSLGQRATISCRASKSVSILGTHLIHWYQQKPGQPPKLLIYAASNLESGVPARFSGSGSETVFTLNIHPVEEEDAATYFCQQSIEDPWTFGCGTKLGIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 17 - protein, 508 aa, synthetic construct
  REGION 1..508: note = hCAR-Vb2-(V1-46*01_1_V7-3*01_1) Complete CAR
MLRLLLALNLFPSIQVTGEVQLVQSGADLVRPGASLKLSCTASGFNIKSAYMHWVRQAPGQCPEWLGRIDPATGKTKYAPKFQGRVTITADTSSNTAYLQLSSLTSEDTAIYYCTRSLNWDYGLDYWGQGTLVTVSSGSTSGSGKPGSGEGSTKGDIVLTQSPASLAVSLGERATISCRASKSVSILGTHLIHWYQQKPGQPPKLLIYAASNLESGVPDRFSGSGSGTDFTLTISRVEAEDVAVYYCQQSIEDPWTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 18 - protein, 508 aa, synthetic construct
  REGION 1..508: note = hCAR-Vb2-(V1-46*01_1_V7-3*01_2) Complete CAR
MLRLLLALNLFPSIQVTGEVQLVQSGADLVRPGASLKLSCTASGFNIKSAYMHWVRQAPGQCPEWLGRIDPATGKTKYAPKFQGRVTITADTSSNTAYLQLSSLTSEDTAIYYCTRSLNWDYGLDYWGQGTLVTVSSGSTSGSGKPGSGEGSTKGDIVLTQSPASLAVSPGERATISCRASKSVSILGTHLIHWYQQKPGQPPKLLIYAASNLESGVPDRFSGSGSGTDFTLTISRVEAEDVAVYYCQQSIEDPWTFGCGTKVEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 19 - protein, 508 aa, synthetic construct
  REGION 1..508: note = hCAR-Vb2-(V1-46*01_1_V4-1*01_1) Complete CAR
MLRLLLALNLFPSIQVTGEVQLVQSGADLVRPGASLKLSCTASGFNIKSAYMHWVRQAPGQCPEWLGRIDPATGKTKYAPKFQGRVTITADTSSNTAYLQLSSLTSEDTAIYYCTRSLNWDYGLDYWGQGTLVTVSSGSTSGSGKPGSGEGSTKGDIVMTQSPDSLAVSLGERATINCRASKSVSILGTHLIHWYQQKPGQPPKLLIYAASNLESGVPDRFSGSGSGTDFTLTISSLQAEDVAVYYCQQSIEDPWTFGCGTKVEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 20 - protein, 508 aa, synthetic construct
  REGION 1..508: note = hCAR-Vb2-(V1-46*01_1_V3D-20*02) Complete CAR
MLRLLLALNLFPSIQVTGEVQLVQSGADLVRPGASLKLSCTASGFNIKSAYMHWVRQAPGQCPEWLGRIDPATGKTKYAPKFQGRVTITADTSSNTAYLQLSSLTSEDTAIYYCTRSLNWDYGLDYWGQGTLVTVSSGSTSGSGKPGSGEGSTKGEIVLTQSPATLSLSPGERATLSCRASKSVSILGTHLIHWYQQKPGQSPRLLIYAASNLESGVPDRFSGSGSGTDFTLTISRLEAEDVAVYYCQQSIEDPWTFGCGTKVEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 21 - protein, 508 aa, synthetic construct
  REGION 1..508: note = hCAR-Vb2-(V1-2*06_V7-3*01_1) Complete CAR
MLRLLLALNLFPSIQVTGQVQLVQSGAEVKKPGASVKVSCKASGFNIKSAYMHWVRQAPGQCLEWMGRIDPATGKTNYAQKFQGRVTMTRDTSISTAYMELSRLRSDDTAVYYCARSLNWDYGLDYWGQGTLVTVSSGSTSGSGKPGSGEGSTKGDIVLTQSPASLAVSLGERATISCRASKSVSILGTHLIHWYQQKPGQPPKLLIYAASNLESGVPDRFSGSGSGTDFTLTISRVEAEDVAVYYCQQSIEDPWTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 22 - protein, 508 aa, synthetic construct
  REGION 1..508: note = hCAR-Vb2-(V1-2*06_V7-3*01_2) Complete CAR
MLRLLLALNLFPSIQVTGQVQLVQSGAEVKKPGASVKVSCKASGFNIKSAYMHWVRQAPGQCLEWMGRIDPATGKTNYAQKFQGRVTMTRDTSISTAYMELSRLRSDDTAVYYCARSLNWDYGLDYWGQGTLVTVSSGSTSGSGKPGSGEGSTKGDIVLTQSPASLAVSPGERATISCRASKSVSILGTHLIHWYQQKPGQPPKLLIYAASNLESGVPDRFSGSGSGTDFTLTISRVEAEDVAVYYCQQSIEDPWTFGCGTKVEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 23 - protein, 508 aa, synthetic construct
  REGION 1..508: note = hCAR-Vb2-(V1-2*06_V4-1*01_1) Complete CAR
MLRLLLALNLFPSIQVTGQVQLVQSGAEVKKPGASVKVSCKASGFNIKSAYMHWVRQAPGQCLEWMGRIDPATGKTNYAQKFQGRVTMTRDTSISTAYMELSRLRSDDTAVYYCARSLNWDYGLDYWGQGTLVTVSSGSTSGSGKPGSGEGSTKGDIVMTQSPDSLAVSLGERATINCRASKSVSILGTHLIHWYQQKPGQPPKLLIYAASNLESGVPDRFSGSGSGTDFTLTISSLQAEDVAVYYCQQSIEDPWTFGCGTKVEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 24 - protein, 508 aa, synthetic construct
  REGION 1..508: note = hCAR-Vb2-(V1-2*06_V3D-20*02) Complete CAR
MLRLLLALNLFPSIQVTGQVQLVQSGAEVKKPGASVKVSCKASGFNIKSAYMHWVRQAPGQCLEWMGRIDPATGKTNYAQKFQGRVTMTRDTSISTAYMELSRLRSDDTAVYYCARSLNWDYGLDYWGQGTLVTVSSGSTSGSGKPGSGEGSTKGEIVLTQSPATLSLSPGERATLSCRASKSVSILGTHLIHWYQQKPGQSPRLLIYAASNLESGVPDRFSGSGSGTDFTLTISRLEAEDVAVYYCQQSIEDPWTFGCGTKVEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 25 - protein, 508 aa, synthetic construct
  REGION 1..508: note = hCAR-Vb2-(V1-3*01_V7-3*01_1) Complete CAR
MLRLLLALNLFPSIQVTGQVQLVQSGAEVKKPGASVKVSCKASGFNIKSAAMHWVRQAPGQCLEWMGWIDPATGKTKYSQKFQGRVTITRDTSASTAYMELSSLRSEDTAVYYCARSLNWDYGLDYWGQGTLVTVSSGSTSGSGKPGSGEGSTKGDIVLTQSPASLAVSLGERATISCRASKSVSILGTHLIHWYQQKPGQPPKLLIYAASNLESGVPDRFSGSGSGTDFTLTISRVEAEDVAVYYCQQSIEDPWTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 26 - protein, 508 aa, synthetic construct
  REGION 1..508: note = hCAR-Vb2-(V1-3*01_V7-3*01_2) Complete CAR
MLRLLLALNLFPSIQVTGQVQLVQSGAEVKKPGASVKVSCKASGFNIKSAAMHWVRQAPGQCLEWMGWIDPATGKTKYSQKFQGRVTITRDTSASTAYMELSSLRSEDTAVYYCARSLNWDYGLDYWGQGTLVTVSSGSTSGSGKPGSGEGSTKGDIVLTQSPASLAVSPGERATISCRASKSVSILGTHLIHWYQQKPGQPPKLLIYAASNLESGVPDRFSGSGSGTDFTLTISRVEAEDVAVYYCQQSIEDPWTFGCGTKVEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 27 - protein, 508 aa, synthetic construct
  REGION 1..508: note = hCAR-Vb2-(V1-3*01_V4-1*01_1) Complete CAR
MLRLLLALNLFPSIQVTGQVQLVQSGAEVKKPGASVKVSCKASGFNIKSAAMHWVRQAPGQCLEWMGWIDPATGKTKYSQKFQGRVTITRDTSASTAYMELSSLRSEDTAVYYCARSLNWDYGLDYWGQGTLVTVSSGSTSGSGKPGSGEGSTKGDIVMTQSPDSLAVSLGERATINCRASKSVSILGTHLIHWYQQKPGQPPKLLIYAASNLESGVPDRFSGSGSGTDFTLTISSLQAEDVAVYYCQQSIEDPWTFGCGTKVEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 28 - protein, 508 aa, synthetic construct
  REGION 1..508: note = hCAR-Vb2-(V1-3*01_V3D-20*02) Complete CAR
MLRLLLALNLFPSIQVTGQVQLVQSGAEVKKPGASVKVSCKASGFNIKSAAMHWVRQAPGQCLEWMGWIDPATGKTKYSQKFQGRVTITRDTSASTAYMELSSLRSEDTAVYYCARSLNWDYGLDYWGQGTLVTVSSGSTSGSGKPGSGEGSTKGEIVLTQSPATLSLSPGERATLSCRASKSVSILGTHLIHWYQQKPGQSPRLLIYAASNLESGVPDRFSGSGSGTDFTLTISRLEAEDVAVYYCQQSIEDPWTFGCGTKVEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 29 - protein, 508 aa, synthetic construct
  REGION 1..508: note = hCAR-Vb2-(V1-46*01_2_V4-1*01_2) Complete CAR
MLRLLLALNLFPSIQVTGQVQLVQSGAEVKKPGASVKVSCKASGFNIKSAYMHWVRQAPGQCLEWLGRIDPATGKTKYAPKFQGRATITADTSSNTAYMELSSLRSEDTAVYYCTRSLNWDYGLDYWGQGTTVTVSSGSTSGSGKPGSGEGSTKGDIVLTQSPDSLAVSLGERATINCKASKSVSILGTHLIHWYQQKPGQPPKLLIYAASNLESGVPDRFSGSGSETVFTLTISSLQAEDVATYFCQQSIEDPWTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 30 - protein, 508 aa, synthetic construct
  REGION 1..508: note = hCAR-Vb2-(V1-46*01_2_V4-1*01_3) Complete CAR
MLRLLLALNLFPSIQVTGQVQLVQSGAEVKKPGASVKVSCKASGFNIKSAYMHWVRQAPGQCLEWLGRIDPATGKTKYAPKFQGRATITADTSSNTAYMELSSLRSEDTAVYYCTRSLNWDYGLDYWGQGTTVTVSSGSTSGSGKPGSGEGSTKGDIVLTQSPDSLAVSLGERATINCKASKSVSILGTHLIHWYQQKPGQPPKLLIYAASNRESGVPDRFSGSGSETDFTLTISSLQAEDVAVYYCQQSIEDPWTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 31 - protein, 508 aa, synthetic construct
  REGION 1..508: note = hCAR-Vb2-(V1-46*01_2_V1-39*01_1) Complete CAR
MLRLLLALNLFPSIQVTGQVQLVQSGAEVKKPGASVKVSCKASGFNIKSAYMHWVRQAPGQCLEWLGRIDPATGKTKYAPKFQGRATITADTSSNTAYMELSSLRSEDTAVYYCTRSLNWDYGLDYWGQGTTVTVSSGSTSGSGKPGSGEGSTKGDIQLTQSPSSLSASVGDRVTITCRASKSVSILGTHLIHWYQQKPGKAPKLLIYAASNLESGVPSRFSGSGSETVFTLTISSLQPEDFATYFCQQSIEDPWTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 32 - protein, 508 aa, synthetic construct
  REGION 1..508: note = hCAR-Vb2-(V1-46*01_2_V1-39*01_2) Complete CAR
MLRLLLALNLFPSIQVTGQVQLVQSGAEVKKPGASVKVSCKASGFNIKSAYMHWVRQAPGQCLEWLGRIDPATGKTKYAPKFQGRATITADTSSNTAYMELSSLRSEDTAVYYCTRSLNWDYGLDYWGQGTTVTVSSGSTSGSGKPGSGEGSTKGDIQLTQSPSSLSASVGDRVTITCRASKSVSILGTHLIHWYQQKPGKAPKLLIYAASNLESGVPSRFSGSGSETDFTLTISSLQPEDFATYYCQQSIEDPWTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 33 - protein, 508 aa, synthetic construct
  REGION 1..508: note = hCAR-Vb2-(V1-46*01_3_V4-1*01_2) Complete CAR
MLRLLLALNLFPSIQVTGQVQLVQSGAEVKKPGASVKVSCKASGFNIKSAYMHWVRQAPGQCLEWMGRIDPATGKTKYAPKFQGRVTMTADTSTSTAYMELSSLRSEDTAVYYCTRSLNWDYGLDYWGQGTTVTVSSGSTSGSGKPGSGEGSTKGDIVLTQSPDSLAVSLGERATINCKASKSVSILGTHLIHWYQQKPGQPPKLLIYAASNLESGVPDRFSGSGSETVFTLTISSLQAEDVATYFCQQSIEDPWTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 34 - protein, 508 aa, synthetic construct
  REGION 1..508: note = hCAR-Vb2-(V1-46*01_3_V4-1*01_3) Complete CAR
MLRLLLALNLFPSIQVTGQVQLVQSGAEVKKPGASVKVSCKASGFNIKSAYMHWVRQAPGQCLEWMGRIDPATGKTKYAPKFQGRVTMTADTSTSTAYMELSSLRSEDTAVYYCTRSLNWDYGLDYWGQGTTVTVSSGSTSGSGKPGSGEGSTKGDIVLTQSPDSLAVSLGERATINCKASKSVSILGTHLIHWYQQKPGQPPKLLIYAASNRESGVPDRFSGSGSETDFTLTISSLQAEDVAVYYCQQSIEDPWTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 35 - protein, 508 aa, synthetic construct
  REGION 1..508: note = hCAR-Vb2-(V1-46*01_3_V1-39*01_1) Complete CAR
MLRLLLALNLFPSIQVTGQVQLVQSGAEVKKPGASVKVSCKASGFNIKSAYMHWVRQAPGQCLEWMGRIDPATGKTKYAPKFQGRVTMTADTSTSTAYMELSSLRSEDTAVYYCTRSLNWDYGLDYWGQGTTVTVSSGSTSGSGKPGSGEGSTKGDIQLTQSPSSLSASVGDRVTITCRASKSVSILGTHLIHWYQQKPGKAPKLLIYAASNLESGVPSRFSGSGSETVFTLTISSLQPEDFATYFCQQSIEDPWTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 36 - protein, 508 aa, synthetic construct
  REGION 1..508: note = hCAR-Vb2-(V1-46*01_3_V1-39*01_2) Complete CAR
MLRLLLALNLFPSIQVTGQVQLVQSGAEVKKPGASVKVSCKASGFNIKSAYMHWVRQAPGQCLEWMGRIDPATGKTKYAPKFQGRVTMTADTSTSTAYMELSSLRSEDTAVYYCTRSLNWDYGLDYWGQGTTVTVSSGSTSGSGKPGSGEGSTKGDIQLTQSPSSLSASVGDRVTITCRASKSVSILGTHLIHWYQQKPGKAPKLLIYAASNLESGVPSRFSGSGSETDFTLTISSLQPEDFATYYCQQSIEDPWTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 37 - protein, 508 aa, synthetic construct
  REGION 1..508: note = hCAR-Vb2-(V7-4-1*02_1_V4-1*01_2) Complete CAR
MLRLLLALNLFPSIQVTGQVQLQQSGPGLVKPSETLSLTCTASGFNIKSAYMHWVRQPPGKCLEWLGRIDPATGKTKYAPSLKSRATISADTSSNTASLKLSSVTAADTAVYYCTRSLNWDYGLDYWGQGTTVTVSSGSTSGSGKPGSGEGSTKGDIVLTQSPDSLAVSLGERATINCKASKSVSILGTHLIHWYQQKPGQPPKLLIYAASNLESGVPDRFSGSGSETVFTLTISSLQAEDVATYFCQQSIEDPWTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 38 - protein, 508 aa, synthetic construct
  REGION 1..508: note = hCAR-Vb2-(V7-4-1*02_1_V4-1*01_3) Complete CAR
MLRLLLALNLFPSIQVTGQVQLQQSGPGLVKPSETLSLTCTASGFNIKSAYMHWVRQPPGKCLEWLGRIDPATGKTKYAPSLKSRATISADTSSNTASLKLSSVTAADTAVYYCTRSLNWDYGLDYWGQGTTVTVSSGSTSGSGKPGSGEGSTKGDIVLTQSPDSLAVSLGERATINCKASKSVSILGTHLIHWYQQKPGQPPKLLIYAASNRESGVPDRFSGSGSETDFTLTISSLQAEDVAVYYCQQSIEDPWTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 39 - protein, 508 aa, synthetic construct
  REGION 1..508: note = hCAR-Vb2-(V7-4-1*02_1_V1-39*01_1) Complete CAR
MLRLLLALNLFPSIQVTGQVQLQQSGPGLVKPSETLSLTCTASGFNIKSAYMHWVRQPPGKCLEWLGRIDPATGKTKYAPSLKSRATISADTSSNTASLKLSSVTAADTAVYYCTRSLNWDYGLDYWGQGTTVTVSSGSTSGSGKPGSGEGSTKGDIQLTQSPSSLSASVGDRVTITCRASKSVSILGTHLIHWYQQKPGKAPKLLIYAASNLESGVPSRFSGSGSETVFTLTISSLQPEDFATYFCQQSIEDPWTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 40 - protein, 508 aa, synthetic construct
  REGION 1..508: note = hCAR-Vb2-(V7-4-1*02_1_V1-39*01_2) Complete CAR
MLRLLLALNLFPSIQVTGQVQLQQSGPGLVKPSETLSLTCTASGFNIKSAYMHWVRQPPGKCLEWLGRIDPATGKTKYAPSLKSRATISADTSSNTASLKLSSVTAADTAVYYCTRSLNWDYGLDYWGQGTTVTVSSGSTSGSGKPGSGEGSTKGDIQLTQSPSSLSASVGDRVTITCRASKSVSILGTHLIHWYQQKPGKAPKLLIYAASNLESGVPSRFSGSGSETDFTLTISSLQPEDFATYYCQQSIEDPWTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 41 - protein, 508 aa, synthetic construct
  REGION 1..508: note = hCAR-Vb2-(V7-4-1*02_2_V4-1*01_2) Complete CAR
MLRLLLALNLFPSIQVTGQVQLQESGPGLVKPSETLSLTCTASGFNIKSAYMHWIRQPPGKCLEWIGRIDPATGKTKYAPSLKSRVTISADTSKNQASLKLSSVTAADTAVYYCTRSLNWDYGLDYWGQGTTVTVSSGSTSGSGKPGSGEGSTKGDIVLTQSPDSLAVSLGERATINCKASKSVSILGTHLIHWYQQKPGQPPKLLIYAASNLESGVPDRFSGSGSETVFTLTISSLQAEDVATYFCQQSIEDPWTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 42 - protein, 508 aa, synthetic construct
  REGION 1..508: note = hCAR-Vb2-(V7-4-1*02_2_V4-1*01_3) Complete CAR
MLRLLLALNLFPSIQVTGQVQLQESGPGLVKPSETLSLTCTASGFNIKSAYMHWIRQPPGKCLEWIGRIDPATGKTKYAPSLKSRVTISADTSKNQASLKLSSVTAADTAVYYCTRSLNWDYGLDYWGQGTTVTVSSGSTSGSGKPGSGEGSTKGDIVLTQSPDSLAVSLGERATINCKASKSVSILGTHLIHWYQQKPGQPPKLLIYAASNRESGVPDRFSGSGSETDFTLTISSLQAEDVAVYYCQQSIEDPWTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 43 - protein, 508 aa, synthetic construct
  REGION 1..508: note = hCAR-Vb2-(V7-4-1*02_2_V1-39*01_1) Complete CAR
MLRLLLALNLFPSIQVTGQVQLQESGPGLVKPSETLSLTCTASGFNIKSAYMHWIRQPPGKCLEWIGRIDPATGKTKYAPSLKSRVTISADTSKNQASLKLSSVTAADTAVYYCTRSLNWDYGLDYWGQGTTVTVSSGSTSGSGKPGSGEGSTKGDIQLTQSPSSLSASVGDRVTITCRASKSVSILGTHLIHWYQQKPGKAPKLLIYAASNLESGVPSRFSGSGSETVFTLTISSLQPEDFATYFCQQSIEDPWTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 44 - protein, 508 aa, synthetic construct
  REGION 1..508: note = hCAR-Vb2-(V7-4-1*02_2_V1-39*01_2) Complete CAR
MLRLLLALNLFPSIQVTGQVQLQESGPGLVKPSETLSLTCTASGFNIKSAYMHWIRQPPGKCLEWIGRIDPATGKTKYAPSLKSRVTISADTSKNQASLKLSSVTAADTAVYYCTRSLNWDYGLDYWGQGTTVTVSSGSTSGSGKPGSGEGSTKGDIQLTQSPSSLSASVGDRVTITCRASKSVSILGTHLIHWYQQKPGKAPKLLIYAASNLESGVPSRFSGSGSETDFTLTISSLQPEDFATYYCQQSIEDPWTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 45 - protein, 119 aa, synthetic construct
  REGION 1..119: note = hCAR-Vb2-(V1-46*01_1_V7-3*01_1) VH
EVQLVQSGADLVRPGASLKLSCTASGFNIKSAYMHWVRQAPGQCPEWLGRIDPATGKTKYAPKFQGRVTITADTSSNTAYLQLSSLTSEDTAIYYCTRSLNWDYGLDYWGQGTLVTVSS

SEQ ID NO: 46 - protein, 119 aa, synthetic construct; identical to SEQ ID NO: 45
  REGION 1..119: note = hCAR-Vb2-(V1-46*01_1_V7-3*01_2) VH

SEQ ID NO: 47 - protein, 119 aa, synthetic construct; identical to SEQ ID NO: 45
  REGION 1..119: note = hCAR-Vb2-(V1-46*01_1_V4-1*01_1) VH

SEQ ID NO: 48 - protein, 119 aa, synthetic construct; identical to SEQ ID NO: 45
  REGION 1..119: note = hCAR-Vb2-(V1-46*01_1_V3D-20*02) VH

SEQ ID NO: 49 - protein, 119 aa, synthetic construct
  REGION 1..119: note = hCAR-Vb2-(V1-2*06_V7-3*01_1) VH
QVQLVQSGAEVKKPGASVKVSCKASGFNIKSAYMHWVRQAPGQCLEWMGRIDPATGKTNYAQKFQGRVTMTRDTSISTAYMELSRLRSDDTAVYYCARSLNWDYGLDYWGQGTLVTVSS

SEQ ID NO: 50 - protein, 119 aa, synthetic construct; identical to SEQ ID NO: 49
  REGION 1..119: note = hCAR-Vb2-(V1-2*06_V7-3*01_2) VH

SEQ ID NO: 51 - protein, 119 aa, synthetic construct; identical to SEQ ID NO: 49
  REGION 1..119: note = hCAR-Vb2-(V1-2*06_V4-1*01_1) VH

SEQ ID NO: 52 - protein, 119 aa, synthetic construct; identical to SEQ ID NO: 49
  REGION 1..119: note = hCAR-Vb2-(V1-2*06_V3D-20*02) VH

SEQ ID NO: 53 - protein, 119 aa, synthetic construct
  REGION 1..119: note = hCAR-Vb2-(V1-3*01_V7-3*01_1) VH
QVQLVQSGAEVKKPGASVKVSCKASGFNIKSAAMHWVRQAPGQCLEWMGWIDPATGKTKYSQKFQGRVTITRDTSASTAYMELSSLRSEDTAVYYCARSLNWDYGLDYWGQGTLVTVSS

SEQ ID NO: 54 - protein, 119 aa, synthetic construct; identical to SEQ ID NO: 53
  REGION 1..119: note = hCAR-Vb2-(V1-3*01_V7-3*01_2) VH

SEQ ID NO: 55 - protein, 119 aa, synthetic construct; identical to SEQ ID NO: 53
  REGION 1..119: note = hCAR-Vb2-(V1-3*01_V4-1*01_1) VH

SEQ ID NO: 56 - protein, 119 aa, synthetic construct; identical to SEQ ID NO: 53
  REGION 1..119: note = hCAR-Vb2-(V1-3*01_V3D-20*02) VH

SEQ ID NO: 57 - protein, 119 aa, synthetic construct
  REGION 1..119: note = hCAR-Vb2-(V1-46*01_2_V4-1*01_2) VH
QVQLVQSGAEVKKPGASVKVSCKASGFNIKSAYMHWVRQAPGQCLEWLGRIDPATGKTKYAPKFQGRATITADTSSNTAYMELSSLRSEDTAVYYCTRSLNWDYGLDYWGQGTTVTVSS

SEQ ID NO: 58 - protein, 119 aa, synthetic construct; identical to SEQ ID NO: 57
  REGION 1..119: note = hCAR-Vb2-(V1-46*01_2_V4-1*01_3) VH

SEQ ID NO: 59 - protein, 119 aa, synthetic construct; identical to SEQ ID NO: 57
  REGION 1..119: note = hCAR-Vb2-(V1-46*01_2_V1-39*01_1) VH

SEQ ID NO: 60 - protein, 119 aa, synthetic construct; identical to SEQ ID NO: 57
  REGION 1..119: note = hCAR-Vb2-(V1-46*01_2_V1-39*01_2) VH

SEQ ID NO: 61 - protein, 119 aa, synthetic construct
  REGION 1..119: note = hCAR-Vb2-(V1-46*01_3_V4-1*01_2) VH
QVQLVQSGAEVKKPGASVKVSCKASGFNIKSAYMHWVRQAPGQCLEWMGRIDPATGKTKYAPKFQGRVTMTADTSTSTAYMELSSLRSEDTAVYYCTRSLNWDYGLDYWGQGTTVTVSS

SEQ ID NO: 62 - protein, 119 aa, synthetic construct; identical to SEQ ID NO: 61
  REGION 1..119: note = hCAR-Vb2-(V1-46*01_3_V4-1*01_3) VH

SEQ ID NO: 63 - protein, 119 aa, synthetic construct; identical to SEQ ID NO: 61
  REGION 1..119: note = hCAR-Vb2-(V1-46*01_3_V1-39*01_1) VH

SEQ ID NO: 64 - protein, 119 aa, synthetic construct; identical to SEQ ID NO: 61
  REGION 1..119: note = hCAR-Vb2-(V1-46*01_3_V1-39*01_2) VH

SEQ ID NO: 65 - protein, 119 aa, synthetic construct
  REGION 1..119: note = hCAR-Vb2-(V7-4-1*02_1_V4-1*01_2) VH
QVQLQQSGPGLVKPSETLSLTCTASGFNIKSAYMHWVRQPPGKCLEWLGRIDPATGKTKYAPSLKSRATISADTSSNTASLKLSSVTAADTAVYYCTRSLNWDYGLDYWGQGTTVTVSS

SEQ ID NO: 66 - protein, 119 aa, synthetic construct; identical to SEQ ID NO: 65
  REGION 1..119: note = hCAR-Vb2-(V7-4-1*02_1_V4-1*01_3) VH

SEQ ID NO: 67 - protein, 119 aa, synthetic construct; identical to SEQ ID NO: 65
  REGION 1..119: note = hCAR-Vb2-(V7-4-1*02_1_V1-39*01_1) VH

SEQ ID NO: 68 - protein, 119 aa, synthetic construct; identical to SEQ ID NO: 65
  REGION 1..119: note = hCAR-Vb2-(V7-4-1*02_1_V1-39*01_2) VH

SEQ ID NO: 69 - protein, 119 aa, synthetic construct
  REGION 1..119: note = hCAR-Vb2-(V7-4-1*02_2_V4-1*01_2) VH
QVQLQESGPGLVKPSETLSLTCTASGFNIKSAYMHWIRQPPGKCLEWIGRIDPATGKTKYAPSLKSRVTISADTSKNQASLKLSSVTAADTAVYYCTRSLNWDYGLDYWGQGTTVTVSS

SEQ ID NO: 70 - protein, 119 aa, synthetic construct; identical to SEQ ID NO: 69
  REGION 1..119: note = hCAR-Vb2-(V7-4-1*02_2_V4-1*01_3) VH

SEQ ID NO: 71 - protein, 119 aa, synthetic construct; identical to SEQ ID NO: 69
  REGION 1..119: note = hCAR-Vb2-(V7-4-1*02_2_V1-39*01_1) VH

SEQ ID NO: 72 - protein, 119 aa, synthetic construct; identical to SEQ ID NO: 69
  REGION 1..119: note = hCAR-Vb2-(V7-4-1*02_2_V1-39*01_2) VH

SEQ ID NO: 73 - protein, 117 aa, synthetic construct
  REGION 1..117: note = VB 7.2 Humanized VH 1
QVHLVQSGAEVKRPGASVKVSCKASGYIFTDYYMHWVRQAPGQCLEWMGWINPSSGYTTYAQKFQDRVTLTADKSSSTAYMELSSLTSEDTAVYYCARSLQLGRDYWGQGTLVTVSS

SEQ ID NO: 74 - protein, 117 aa, synthetic construct
  REGION 1..117: note = VB 7.2 Humanized VH 2
QVQLVQSGAEVKKPGASVKVSCKASGYIFTDYYMHWVRQAPGQCLEWMGWINPSSGYTNYAQKFQGRVTMTRDTSISTAYMELSRLRSDDTAVYYCARSLQLGRDYWGQGTLVTVSS

SEQ ID NO: 75 - protein, 117 aa, synthetic construct
  REGION 1..117: note = VB 7.2 Humanized VH 3
QVQLVQSGAEVKKPGASVKVSCKASGYIFTDYYMHWVRQAPGQCLEWMGIINPSSGYTSYAQKFQGRVTMTRDTSTSTVYMELSSLRSEDTAVYYCARSLQLGRDYWGQGTLVTVSS

SEQ ID NO: 76 - protein, 120 aa, synthetic construct
  REGION 1..120: note = VB 4 Humanized VH 1
QVQLVQSGAEVKKPGASVKVSCKASGYTFTSYYIYWVRQAPGQCLEWLGWIYPGNGGTYYSEKFQGRVTFTADTSTNTAYMELSSLTSEDTAVYFCARGSGDRYNSLAYWGQGTLVTVSS

SEQ ID NO: 77 - protein, 120 aa, synthetic construct
  REGION 1..120: note = VB 4 Humanized VH 2
QVQLVQSGAEVKKPGASVKVSCKASGYTFTSYAMHWVRQAPGQCLEWMGWIYPGNGGTKYSQKFQGRVTITRDTSASTAYMELSSLRSEDTAVYYCARGSGDRYNSLAYWGQGTLVTVSS

SEQ ID NO: 78 - protein, 120 aa, synthetic construct
  REGION 1..120: note = VB 4 Humanized VH 3
QVQLVQSGAEVKKPGASVKVSCKASGYTFTSYYMHWVRQAPGQCLEWMGIIYPGNGGTSYAQKFQGRVTMTRDTSTSTVYMELSSLRSEDTAVYYCARGSGDRYNSLAYWGQGTLVTVSS

SEQ ID NO: 79 - protein, 120 aa, synthetic construct
  REGION 1..120: note = VB 4 Humanized VH 4
QVQLVQSGAEVKKPGASVKVSCKASGYTFTSYGISWVRQAPGQCLEWMGWIYPGNGGTNYAQKLQGRVTMTTDTSTSTAYMELRSLRSDDTAVYYCARGSGDRYNSLAYWGQGTLVTVSS

SEQ ID NO: 80 - protein, 120 aa, synthetic construct
  REGION 1..120: note = VB 4 Humanized VH 5
QVQLVQSGAEVKKPGASVKVSCKASGYTFTSYYMHWVRQAPGQCLEWMGWIYPGNGGTNYAQKFQGRVTMTRDTSISTAYMELSRLRSDDTAVYYCARGSGDRYNSLAYWGQGTLVTVSS

SEQ ID NO: 81 - protein, 120 aa, synthetic construct
  REGION 1..120: note = VB 13.2 Humanized VH 1
QVQLVQSGPEVKKPGASVKVSCKASGYTFTDYYIHWLRQAPGKCLEWIGWVNTNNGDTNYAQRFKGRVSLTRDKSTSTAYMELNSLTSEDTAVYYCARALYTGSYWFAYWGQGTLVTVSS

SEQ ID NO: 82 - protein, 120 aa, synthetic construct
  REGION 1..120: note = VB 13.2 Humanized VH 2
QVQLVQSGAEVKKPGASVKVSCKASGYTFTDYYMHWVRQAPGQCLEWMGWINTNNGDTNYAQKFQGRVTMTRDTSISTAYMELSRLRSDDTAVYYCARALYTGSYWFAYWGQGTLVTVSS

SEQ ID NO: 83 - protein, 120 aa, synthetic construct
  REGION 1..120: note = VB 13.2 Humanized VH 3
QVQLVQSGAEVKKPGASVKVSCKASGYTFTDYYMHWVRQAPGQCLEWMGIINTNNGDTSYAQKFQGRVTMTRDTSTSTVYMELSSLRSEDTAVYYCARALYTGSYWFAYWGQGTLVTVSS

SEQ ID NO: 84 - protein, 120 aa, synthetic construct
  REGION 1..120: note = VB 13.2 Humanized VH 4
QVQLVQSGAEVKKPGASVKVSCKASGYTFTDYYIHWLRQAPGQCLEWIGIVNTNNGDTNYNQKFQGRATLTVDKSSSTAYMELSSLRSEDTAVYYCARALYTGSYWFAYWGQGTLVTVSS

SEQ ID NO: 85 - protein, 120 aa, synthetic construct
  REGION 1..120: note = VB 13.2 Humanized VH 5
QVQLVQSGAEVKKPGASVKVSCKASGYTFTDYYIHWVRQAPGQCLEWMGIVNTNNGDTNYNQKFQGRVTMTVDKSTSTAYMELSSLRSEDTAVYYCARALYTGSYWFAYWGQGTLVTVSS

SEQ ID NO: 86 - protein, 120 aa, synthetic construct
  REGION 1..120: note = VB 13.2 Humanized VH 6
QVQLQQSGPGLVKPSETLSLTCTASGYTFTDYYIHWLRQPPGKCLEWIGIVNTNNGDTNYNQRLKSRATLSVDKSSSTASLKLSSVTAADTAVYYCARALYTGSYWFAYWGQGTLVTVSS

SEQ ID NO: 87 - protein, 120 aa, synthetic construct
  REGION 1..120: note = VB 13.2 Humanized VH 7
QVQLQESGPGLVKPSETLSLTCTASGYTFTDYYIHWIRQPPGKCLEWIGIVNTNNGDTNYNQRLKSRVTISVDKSKNQASLKLSSVTAADTAVYYCARALYTGSYWFAYWGQGTLVTVSS

SEQ ID NO: 88 - protein, 118 aa, synthetic construct
  REGION 1..118: note = VB 14 Humanized VH 1
QVQLVQSGAEVKKPGASVKLSCKASGFKTEDTYMHWVRQAPGQCLEWIGRIDPANGNTKYAPKFQDRVTITADTSANTAYLELSSLRSEDTAVYYCARGSYYYAMDYWGQGTTVTVSS

SEQ ID NO: 89 - protein, 118 aa, synthetic construct
  REGION 1..118: note = VB 14 Humanized VH 2
QVQLVQSGAEVKKPGASVKVSCKASGFKTEDTAMHWVRQAPGQCLEWMGWIDPANGNTKYSQKFQGRVTITRDTSASTAYMELSSLRSEDTAVYYCARGSYYYAMDYWGQGTTVTVSS

SEQ ID NO: 90 - protein, 118 aa, synthetic construct
  REGION 1..118: note = VB 14 Humanized VH 3
QVQLVQSGAEVKKPGASVKVSCKASGFKTEDTYMHWVRQAPGQCLEWMGRIDPANGNTNYAQKFQGRVTMTRDTSISTAYMELSRLRSDDTAVYYCARGSYYYAMDYWGQGTTVTVSS

SEQ ID NO: 91 - protein, 118 aa, synthetic construct
  REGION 1..118: note = VB 14 Humanized VH 4
QVQLVQSGAEVKKPGASVKVSCKASGFKTEDTYMHWVRQAPGQCLEWMGIIDPANGNTSYAQKFQGRVTMTRDTSTSTVYMELSSLRSEDTAVYYCARGSYYYAMDYWGQGTTVTVSS

SEQ ID NO: 92 - protein, 116 aa, synthetic construct
  REGION 1..116: note = VB 22 Humanized VH 1
EVQLVESGGGLVQPGGSLRLSCAASGFDFSRYWMNWVRQAPGKCLEWVSYINSDSNTINYADSVKGRFTISRDNAKNSLYLQMNSLRAEDTALYYCARGGLLRDVWGQGTTVTVSS

SEQ ID NO: 93 - protein, 116 aa, synthetic construct
  REGION 1..116: note = VB 22 Humanized VH 2
EVQLVESGGGLVQPGGSLRLSCAASGFDFSRYSMNWVRQAPGKCLEWVSYINSDSNTIYYADSVKGRFTISRDNAKNSLYLQMNSLRAEDTAVYYCARGGLLRDVWGQGTMVTVSS

SEQ ID NO: 94 - protein, 116 aa, synthetic construct
  REGION 1..116: note = VB 22 Humanized VH 3
EVQLVESGGGLVKPGGSLRLSCAASGFDFSRYSMNWVRQAPGKCLEWVSSINSDSNTIYYADSVKGRFTISRDNAKNSLYLQMNSLRAEDTAVYYCARGGLLRDVWGQGTMVTVSS

SEQ ID NO: 95 - protein, 116 aa, synthetic construct
  REGION 1..116: note = VB 11 Humanized VH 1
QVQLVQSGPEVKRPGASVKISCKASGYRFTDSAMHWVRQAPGQCLEWMGWISSYNGNTNYAQKFQGRVTMTVDTSTSTAYMELSRLTSEDTAIYYCARSRDAMDYWGQGTLVTVSS

SEQ ID NO: 96 - protein, 116 aa, synthetic construct
  REGION 1..116: note = VB 11 Humanized VH 2
QVQLVQSGAEVKKPGASVKVSCKASGYRFTDSGISWVRQAPGQCLEWMGWISSYNGNTNYAQKLQGRVTMTTDTSTSTAYMELRSLRSDDTAVYYCARSRDAMDYWGQGTLVTVSS

SEQ ID NO: 97 - protein, 116 aa, synthetic construct
  REGION 1..116: note = VB 11 Humanized VH 3
QVQLVQSGAEVKKPGASVKVSCKASGYRFTDSYMHWVRQAPGQCLEWMGWISSYNGNTNYAQKFQGRVTMTRDTSISTAYMELSRLRSDDTAVYYCARSRDAMDYWGQGTLVTVSS

SEQ ID NO: 98 - protein, 121 aa, synthetic construct
  REGION 1..121: note = VB 1 Humanized VH 1
QVQLVQSGSELREPGASVKISCKASGYTFTDYYVHWVRQAPGKCLEWMGWINTYTGTPTYAQDFTGRFVFSLDTSASTAYLQISNLKAEDTAVYFCARSWRRGIRGIGFDWGQGTLVTVSS

SEQ ID NO: 99 - protein, 121 aa, synthetic construct
  REGION 1..121: note = VB 1 Humanized VH 2
QVQLVQSGSELKKPGASVKVSCKASGYTFTDYAMNWVRQAPGQCLEWMGWINTYTGTPTYAQGFTGRFVFSLDTSVSTAYLQISSLKAEDTAVYYCARSWRRGIRGIGFDWGQGTLVTVSS

SEQ ID NO: 100 - protein, 117 aa, synthetic construct
  REGION 1..117: note = VB 12 Humanized VH 1
EVQLVESGGGLVKPGGSLRLSCAASGFTFRSYGMSWVRQAPGKCLEWVASISSGGSYTYYADSVKGRFTISRDNAKNSLYLQMSSLRAEDTAIYYCARHGGNFFDYWGQGTLVTVSS

SEQ ID NO: 101 - protein, 117 aa, synthetic construct
  REGION 1..117: note = VB 12 Humanized VH 2
EVQLVESGGGLVKPGGSLRLSCAASGFTFRSYSMNWVRQAPGKCLEWVSSISSGGSYIYYADSVKGRFTISRDNAKNSLYLQMNSLRAEDTAVYYCARHGGNFFDYWGQGTLVTVSS

SEQ ID NO: 102 - protein, 117 aa, synthetic construct
  REGION 1..117: note = VB 12 Humanized VH 3
EVQLVESGGGLVQPGGSLRLSCAASGFTFRSYEMNWVRQAPGKCLEWVSYISSGGSYIYYADSVKGRFTISRDNAKNSLYLQMNSLRAEDTAVYYCARHGGNFFDYWGQGTLVTVSS

SEQ ID NO: 103 - protein, 117 aa, synthetic construct
  REGION 1..117: note = VB 12 Humanized VH 4
QVQLVESGGGLVKPGGSLRLSCAASGFTFRSYYMSWIRQAPGKCLEWVSYISSGGSYIYYADSVKGRFTISRDNAKNSLYLQMNSLRAEDTAVYYCARHGGNFFDYWGQGTLVTVSS

SEQ ID NO: 104 - protein, 117 aa, synthetic construct
  REGION 1..117: note = VB 8 Humanized VH 1
EVQLVESGGGLVQPGGSLRLSCAASGFTFSNFGMHWVRQAPGKCLEWVAYISSGSSTIYYADSVKGRFTISRDNAKNTLFLQMNSLRAEDTAVYYCARRGEGAMDYWGQGTLVTVSS

SEQ ID NO: 105 - protein, 117 aa, synthetic construct
  REGION 1..117: note = VB 8 Humanized VH 2
EVQLVESGGGLVQPGGSLRLSCAASGFTFSNFSMNWVRQAPGKCLEWVSYISSGSSTIYYADSVKGRFTISRDNAKNSLYLQMNSLRAEDTAVYYCARRGEGAMDYWGQGTLVTVSS

SEQ ID NO: 106 - protein, 117 aa, synthetic construct
  REGION 1..117: note = VB 8 Humanized VH 3
EVQLVESGGGLVKPGGSLRLSCAASGFTFSNFSMNWVRQAPGKCLEWVSYISSGSSTIYYADSVKGRFTISRDNAKNSLYLQMNSLRAEDTAVYYCARRGEGAMDYWGQGTLVTVSS

SEQ ID NO: 107 - protein, 121 aa, synthetic construct
  REGION 1..121: note = VB 13.1 Humanized VH 1
EVQLVESGGGLVKPGGSLRLSCAASGFTFRSYAMSWVRQAPGKCLEWVASISNGGDYIYYADSVKGRFTISRDNARNTLYLQMSSLRAEDTAVYYCARPSYYSDPWFFDVWGRGTTVTVSS

SEQ ID NO: 108 - protein, 121 aa, synthetic construct
  REGION 1..121: note = VB 13.1 Humanized VH 2
EVQLVESGGGLVKPGGSLRLSCAASGFTFRSYSMNWVRQAPGKCLEWVSSISNGGDYIYYADSVKGRFTISRDNAKNSLYLQMNSLRAEDTAVYYCARPSYYSDPWFFDVWGRGTLVTVSS

SEQ ID NO: 109 - protein, 121 aa, synthetic construct
  REGION 1..121: note = VB 13.1 Humanized VH 3
EVQLVESGGGLVQPGGSLRLSCAASGFTFRSYAMSWVRQAPGKCLEWVSAISNGGDYTYYADSVKGRFTISRDNSKNTLYLQMNSLRAEDTAVYYCAKPSYYSDPWFFDVWGRGTLVTVSS

SEQ ID NO: 110 - protein, 121 aa, synthetic construct
  REGION 1..121: note = VB 13.1 Humanized VH 4
EVQLVESGGGLVQPGGSLRLSCAASGFTFRSYEMNWVRQAPGKCLEWVSYISNGGDYIYYADSVKGRFTISRDNAKNSLYLQMNSLRAEDTAVYYCARPSYYSDPWFFDVWGRGTLVTVSS

SEQ ID NO: 111 - protein, 118 aa, synthetic construct
  REGION 1..118: note = VB 5.1 Humanized VH 1
QVQLVQSGPELKKPGASVRVSCKASGYTFTDYYIHWVRQAPGRCLEWVGWINPYNGRTGYAQKFKGRVTLTRDKSSSTAYMDLRSLTSEDTAVYYCARWDGSSYFDYWGQGTLVTVSS

SEQ ID NO: 112 - protein, 118 aa, synthetic construct
  REGION 1..118: note = VB 5.1 Humanized VH 2
QVQLVQSGAEVKKPGASVKVSCKASGYTFTDYYMHWVRQAPGQCLEWMGWINPYNGRTNYAQKFQGRVTMTRDTSISTAYMELSRLRSDDTAVYYCARWDGSSYFDYWGQGTLVTVSS

SEQ ID NO: 113 - protein, 118 aa, synthetic construct
  REGION 1..118: note = VB 5.1 Humanized VH 3
QVQLVQSGAEVKKPGASVKVSCKASGYTFTDYYMHWVRQAPGQCLEWMGIINPYNGRTSYAQKFQGRVTMTRDTSTSTVYMELSSLRSEDTAVYYCARWDGSSYFDYWGQGTLVTVSS

SEQ ID NO: 114 - protein, 118 aa, synthetic construct
  REGION 1..118: note = VB 5.1 Humanized VH 4
QVQLVQSGAEVKKPGASVKVSCKASGYTFTDYGISWVRQAPGQCLEWMGWINPYNGRTNYAQKLQGRVTMTTDTSTSTAYMELRSLRSDDTAVYYCARWDGSSYFDYWGQGTLVTVSS

SEQ ID NO: 115 - protein, 118 aa, synthetic construct; identical to SEQ ID NO: 112
  REGION 1..118: note = VB 5.1 Humanized VH 5

SEQ ID NO: 116 - protein, 118 aa, synthetic construct
  REGION 1..118: note = VB 9 Humanized VH 1
QVQLVQSGAELKRPGASVKLSCTASGFNIKNTFMHWVRQAPGQCLEWIGRIDPTNGYTKFAPKFQGRVTLTADTSSNTVYLQLSSLTSEDTAIYYCARDYDAPWFAYWGQGTLVIVSS

SEQ ID NO: 117 - protein, 118 aa, synthetic construct
  REGION 1..118: note = VB 9 Humanized VH 2
QVQLVQSGAEVKKPGASVKVSCKASGFNIKNTYMHWVRQAPGQCLEWMGIIDPTNGYTSYAQKFQGRVTMTRDTSTSTVYMELSSLRSEDTAVYYCARDYDAPWFAYWGQGTLVTVSS

SEQ ID NO: 118 - protein, 118 aa, synthetic construct
  REGION 1..118: note = VB 9 Humanized VH 3
QVQLVQSGAEVKKPGASVKVSCKASGFNIKNTYMHWVRQAPGQCLEWMGRIDPTNGYTNYAQKFQGRVTMTRDTSISTAYMELSRLRSDDTAVYYCARDYDAPWFAYWGQGTLVTVSS

SEQ ID NO: 119 - protein, 118 aa, synthetic construct
  REGION 1..118: note = VB 9 Humanized VH 4
QVQLVQSGAEVKKPGASVKVSCKASGFNIKNTAMHWVRQAPGQCLEWMGWIDPTNGYTKYSQKFQGRVTITRDTSASTAYMELSSLRSEDTAVYYCARDYDAPWFAYWGQGTLVTVSS

SEQ ID NO: 120 - protein, 120 aa, synthetic construct
  REGION 1..120: note = VB 17 Humanized VH 1
QVQLQESGPGLVKPSQTLSLTCSVSGYSITSGYFWNWIRQPPGKCLEWIGYISYDGSTNYNPSLKSRISISVDTSKNQFSLKLNSVTAADTAVYYCASPSPGTGYAVDYWGQGTLVTVSS

SEQ ID NO: 121 - protein, 119 aa, synthetic construct
  REGION 1..119: note = VB 17 Humanized VH 2
QVQLQESGPGLVKPSETLSLTCTVSGYSITSGYWSWIRQPPGKCLEWIGYISYDGSTNYNPSLKSRVTISVDTSKNQFSLKLSSVTAADTAVYYCARPSPGTGYAVDYWGQGTLVTVSS

SEQ ID NO: 122 - protein, 120 aa, synthetic construct
  REGION 1..120: note = VB 17 Humanized VH 3
QVQLQESGPGLVKPSQTLSLTCAVSGYSITSGYYWGWIRQPPGKCLEWIGYISYDGSTYYNPSLKSRVTISVDTSKNQFSLKLSSVTAADTAVYYCARPSPGTGYAVDYWGQGTLVTVSS

SEQ ID NO: 123 - protein, 121 aa, synthetic construct
  REGION 1..121: note = VB 3 Humanized VH 1
QVQLVQSGPEVKKPGASVKISCKASGYTFSGSWMHWVRQAPGKCLEWIGRIYPGDGDTDYSQKFQGRVTLTADKSSSTAYMELSSLTSEDTAVYFCARSGYFNYVPVFDYWGQGTLVTVSS

SEQ ID NO: 124 - protein, 121 aa, synthetic construct
  REGION 1..121: note = VB 3 Humanized VH 2
QVQLVQSGAEVKKPGASVKVSCKASGYTFSGSYMHWVRQAPGQCLEWMGRIYPGDGDTNYAQKFQGRVTMTRDTSISTAYMELSRLRSDDTAVYYCARSGYFNYVPVFDYWGQGTLVTVSS

SEQ ID NO: 125 - protein, 121 aa, synthetic construct
  REGION 1..121: note = VB 3 Humanized VH 3
QVQLVQSGAEVKKPGASVKVSCKASGYTFSGSAMHWVRQAPGQCLEWMGWIYPGDGDTKYSQKFQGRVTITRDTSASTAYMELSSLRSEDTAVYYCARSGYFNYVPVFDYWGQGTLVTVSS

SEQ ID NO: 126 - protein, 123 aa, synthetic construct
  REGION 1..123: note = VB 7.1 Humanized VH 1
EVQLVESGGGLVKPGGSLRLSCAASGFTFSDYYMNWVRQTPGKCLEWVASISGGGSYTYYADSVKGRFTISRDNAKNSLYLQMSSLRAEDTAVYFCARERDIYYGNFNAMVYWGQGTLVTVSS

SEQ ID NO: 127 - protein, 123 aa, synthetic construct
  REGION 1..123: note = VB 7.1 Humanized VH 2
EVQLVESGGGLVKPGGSLRLSCAASGFTFSDYYMNWVRQAPGKCLEWVSSISGGGSYIYYADSVKGRFTISRDNAKNSLYLQMNSLRAEDTAVYYCARERDIYYGNFNAMVYWGRGTLVTVSS

SEQ ID NO: 128 - protein, 123 aa, synthetic construct
  REGION 1..123: note = VB 7.1 Humanized VH 3
EVQLVESGGGLVKPGGSLRLSCAASGFTFSDYSMNWVRQAPGKCLEWVSSISGGGSYIYYADSVKGRFTISRDNAKNSLYLQMNSLRAEDTAVYYCARERDIYYGNFNAMVYWGRGTLVTVSS

SEQ ID NO: 129 - protein, 123 aa, synthetic construct
  REGION 1..123: note = VB 7.1 Humanized VH 4
QVQLVESGGGLVKPGGSLRLSCAASGFTFSDYYMSWIRQAPGKCLEWVSYISGGGSYIYYADSVKGRFTISRDNAKNSLYLQMNSLRAEDTAVYYCARERDIYYGNFNAMVYWGQGTLVTVSS

SEQ ID NO: 130 - protein, 119 aa, synthetic construct
  REGION 1..119: note = VB 5.3 Humanized VH 1
QVQLVQSGPELKKPGASVKISCKASGYAFSSSWMHWVRQAPGQCLEWIGRIYPGDGDTKYAQKFQGRVTLTADKSSSTAYMELSSLTSEDTAVYFCARRGTGGWYFDVWGKGTLVTVSS

SEQ ID NO: 131 - protein, 119 aa, synthetic construct
  REGION 1..119: note = VB 5.3 Humanized VH 2
QVQLVQSGAEVKKPGASVKVSCKASGYAFSSSAMHWVRQAPGQCLEWMGWIYPGDGDTKYSQKFQGRVTITRDTSASTAYMELSSLRSEDTAVYYCARRGTGGWYFDVWGRGTLVTVSS

SEQ ID NO: 132 - protein, 111 aa, synthetic construct
  REGION 1..111: note = hCAR-Vb2-(V1-46*01_1_V7-3*01_1) VL
DIVLTQSPASLAVSLGERATISCRASKSVSILGTHLIHWYQQKPGQPPKLLIYAASNLESGVPDRFSGSGSGTDFTLTISRVEAEDVAVYYCQQSIEDPWTFGCGTKLEIK

SEQ ID NO: 133 - protein, 111 aa, synthetic construct
  REGION 1..111: note = hCAR-Vb2-(V1-46*01_1_V7-3*01_2) VL
DIVLTQSPASLAVSPGERATISCRASKSVSILGTHLIHWYQQKPGQPPKLLIYAASNLESGVPDRFSGSGSGTDFTLTISRVEAEDVAVYYCQQSIEDPWTFGCGTKVEIK

SEQ ID NO: 134 - protein, 111 aa, synthetic construct
  REGION 1..111: note = hCAR-Vb2-(V1-46*01_1_V4-1*01_1) VL
DIVMTQSPDSLAVSLGERATINCRASKSVSILGTHLIHWYQQKPGQPPKLLIYAASNLESGVPDRFSGSGSGTDFTLTISSLQAEDVAVYYCQQSIEDPWTFGCGTKVEIK

SEQ ID NO: 135 - protein, 111 aa, synthetic construct
  REGION 1..111: note = hCAR-Vb2-(V1-46*01_1_V3D-20*02) VL
EIVLTQSPATLSLSPGERATLSCRASKSVSILGTHLIHWYQQKPGQSPRLLIYAASNLESGVPDRFSGSGSGTDFTLTISRLEAEDVAVYYCQQSIEDPWTFGCGTKVEIK

SEQ ID NO: 136 - protein, 111 aa, synthetic construct; identical to SEQ ID NO: 132
  REGION 1..111: note = hCAR-Vb2-(V1-2*06_V7-3*01_1) VL

SEQ ID NO: 137 - protein, 111 aa, synthetic construct; identical to SEQ ID NO: 133
  REGION 1..111: note = hCAR-Vb2-(V1-2*06_V7-3*01_2) VL

SEQ ID NO: 138 - protein, 111 aa, synthetic construct; identical to SEQ ID NO: 134
  REGION 1..111: note = hCAR-Vb2-(V1-2*06_V4-1*01_1) VL

SEQ ID NO: 139 - protein, 111 aa, synthetic construct; identical to SEQ ID NO: 135
  REGION 1..111: note = hCAR-Vb2-(V1-2*06_V3D-20*02) VL

SEQ ID NO: 140 - protein, 111 aa, synthetic construct; identical to SEQ ID NO: 132
  REGION 1..111: note = hCAR-Vb2-(V1-3*01_V7-3*01_1) VL

SEQ ID NO: 141 - protein, 111 aa, synthetic construct; identical to SEQ ID NO: 133
  REGION 1..111: note = hCAR-Vb2-(V1-3*01_V7-3*01_2) VL

SEQ ID NO: 142 - protein, 111 aa, synthetic construct; identical to SEQ ID NO: 134
  REGION 1..111: note = hCAR-Vb2-(V1-3*01_V4-1*01_1) VL

SEQ ID NO: 143 - protein, 111 aa, synthetic construct; identical to SEQ ID NO: 135
  REGION 1..111: note = hCAR-Vb2-(V1-3*01_V3D-20*02) VL

SEQ ID NO: 144 - protein, 111 aa, synthetic construct
  REGION 1..111: note = hCAR-Vb2-(V1-46*01_2_V4-1*01_2) VL
DIVLTQSPDSLAVSLGERATINCKASKSVSILGTHLIHWYQQKPGQPPKLLIYAASNLESGVPDRFSGSGSETVFTLTISSLQAEDVATYFCQQSIEDPWTFGCGTKLEIK

SEQ ID NO: 145 - protein, 111 aa, synthetic construct
  REGION 1..111: note = hCAR-Vb2-(V1-46*01_2_V4-1*01_3) VL
DIVLTQSPDSLAVSLGERATINCKASKSVSILGTHLIHWYQQKPGQPPKLLIYAASNRESGVPDRFSGSGSETDFTLTISSLQAEDVAVYYCQQSIEDPWTFGCGTKLEIK

SEQ ID NO: 146 - protein, 111 aa, synthetic construct
  REGION 1..111: note = hCAR-Vb2-(V1-46*01_2_V1-39*01_1) VL
DIQLTQSPSSLSASVGDRVTITCRASKSVSILGTHLIHWYQQKPGKAPKLLIYAASNLESGVPSRFSGSGSETVFTLTISSLQPEDFATYFCQQSIEDPWTFGCGTKLEIK

SEQ ID NO: 147 - protein, 111 aa, synthetic construct
  REGION 1..111: note = hCAR-Vb2-(V1-46*01_2_V1-39*01_2) VL
DIQLTQSPSSLSASVGDRVTITCRASKSVSILGTHLIHWYQQKPGKAPKLLIYAASNLESGVPSRFSGSGSETDFTLTISSLQPEDFATYYCQQSIEDPWTFGCGTKLEIK

SEQ ID NO: 148 - protein, 111 aa, synthetic construct; identical to SEQ ID NO: 144
  REGION 1..111: note = hCAR-Vb2-(V1-46*01_3_V4-1*01_2) VL

SEQ ID NO: 149 - protein, 111 aa, synthetic construct; identical to SEQ ID NO: 145
  REGION 1..111: note = hCAR-Vb2-(V1-46*01_3_V4-1*01_3) VL

SEQ ID NO: 150 - protein, 111 aa, synthetic construct; identical to SEQ ID NO: 146
  REGION 1..111: note = hCAR-Vb2-(V1-46*01_3_V1-39*01_1) VL

SEQ ID NO: 151 - protein, 111 aa, synthetic construct; identical to SEQ ID NO: 147
  REGION 1..111: note = hCAR-Vb2-(V1-46*01_3_V1-39*01_2) VL

SEQ ID NO: 152 - protein, 111 aa, synthetic construct; identical to SEQ ID NO: 144
  REGION 1..111: note = hCAR-Vb2-(V7-4-1*02_1_V4-1*01_2) VL

SEQ ID NO: 153 - protein, 111 aa, synthetic construct; identical to SEQ ID NO: 145
  REGION 1..111: note = hCAR-Vb2-(V7-4-1*02_1_V4-1*01_3) VL

SEQ ID NO: 154 - protein, 111 aa, synthetic construct; identical to SEQ ID NO: 146
  REGION 1..111: note = hCAR-Vb2-(V7-4-1*02_1_V1-39*01_1) VL

SEQ ID NO: 155 - protein, 111 aa, synthetic construct; identical to SEQ ID NO: 147
  REGION 1..111: note = hCAR-Vb2-(V7-4-1*02_1_V1-39*01_2) VL

SEQ ID NO: 156 - protein, 111 aa, synthetic construct; identical to SEQ ID NO: 144
  REGION 1..111: note = hCAR-Vb2-(V7-4-1*02_2_V4-1*01_2) VL

SEQ ID NO: 157 - protein, 111 aa, synthetic construct; identical to SEQ ID NO: 145
  REGION 1..111: note = hCAR-Vb2-(V7-4-1*02_2_V4-1*01_3) VL

SEQ ID NO: 158 - protein, 111 aa, synthetic construct; identical to SEQ ID NO: 146
  REGION 1..111: note = hCAR-Vb2-(V7-4-1*02_2_V1-39*01_1) VL

SEQ ID NO: 159 - protein, 111 aa, synthetic construct; identical to SEQ ID NO: 147
  REGION 1..111: note = hCAR-Vb2-(V7-4-1*02_2_V1-39*01_2) VL

SEQ ID NO: 160 - protein, 107 aa, synthetic construct
  REGION 1..107: note = VB 7.2 Humanized VL 1
DIQMTQSPSFLSASVGDRVTITCKASQDVYTAVAWYQQKPGQAPKLLIYSASNRYTGVPSRFSGSGSGTDFTLTISSLQAEDLATYYCQQHYTTPRTFGCGTKVEIK

SEQ ID NO: 161 - protein, 107 aa, synthetic construct
  REGION 1..107: note = VB 7.2 Humanized VL 2
DIQMTQSPSSLSASVGDRVTITCKASQDVYTAVAWYQQKPGKAPKLLIYSASNRYTGVPSRFSGSGSGTDFTFTISSLQPEDIATYYCQQHYTTPRTFGCGTKLEIK

SEQ ID NO: 162 - protein, 107 aa, synthetic construct
  REGION 1..107: note = VB 7.2 Humanized VL 3
DIQMTQSPSSVSASVGDRVTITCKASQDVYTAVAWYQQKPGKAPKLLIYSASNRYTGVPSRFSGSGSGTDFTLTISSLQPEDFATYYCQQHYTTPRTFGCGTKLEIK

SEQ ID NO: 163 - protein, 107 aa, synthetic construct
  REGION 1..107: note = VB 7.2 Humanized VL 4
AIQLTQSPSSLSASVGDRVTITCKASQDVYTAVAWYQQKPGKAPKLLIYSASNRYTGVPSRFSGSGSGTDFTLTISSLQPEDFATYYCQQHYTTPRTFGCGTKLEIK

SEQ ID NO: 164 - protein, 107 aa, synthetic construct
  REGION 1..107: note = VB 4 murine VL
DIQMTQSPHSLSASLGETVSIECLASEGISNFLAWYQQKPGKSPQLLIYYTSSLQDGVPSRFSGSGSGTQYSLKISNMQPEDEGVYYCQQGYKFPRTFGCGTKLELK

SEQ ID NO: 165 - protein, 107 aa, synthetic construct
  REGION 1..107: note = VB 4 murine VL1
DIQMTQSPSSLSASVGDTVTITCLASEGISNFLAWYQQKPGKAPKLLIYYTSSLQDGVPSRFSGSGSGTDFSLTISNLQPEDFGTYYCQQGYKFPRTFGCGTKVEIK

SEQ ID NO: 166 - protein, 107 aa, synthetic construct
  REGION 1..107: note = VB 4 Humanized VL 1
DIQMTQSPSSVSASVGDRVTITCLASEGISNFLAWYQQKPGKAPKLLIYYTSSLQDGVPSRFSGSGSGTDFTLTISSLQPEDFATYYCQQGYKFPRTFGCGTKLEIK

SEQ ID NO: 167 - protein, 107 aa, synthetic construct
  REGION 1..107: note = VB 4 Humanized VL 2
DIQMTQSPSSLSASVGDRVTITCLASEGISNFLAWFQQKPGKAPKSLIYYTSSLQDGVPSRFSGSGSGTDFTLTISSLQPEDFATYYCQQGYKFPRTFGCGTKLEIK

SEQ ID NO: 168 - protein, 108 aa, synthetic construct
  REGION 1..108: note = VB 13.2 Humanized VL 1
EIVLTQSPATLSVSPGEKVTITCSASSSISSTNLHWYQQKPGQSPRLLIYGTSNLASGVPDRFSGSGSGTDFTLTISSLEAEDFATYYCQQWSRYPLTFGCGTKLEIK

SEQ ID NO: 169 - protein, 108 aa, synthetic construct
  REGION 1..108: note = VB 13.2 Humanized VL 2
EIVLTQSPATLSLSPGERATLSCSASSSISSTNLHWYQQKPGQAPRLLIYGTSNLASGIPDRFSGSGSGTDFTLTISSLEPEDFAVYYCQQWSRYPLTFGCGTKLEIK

SEQ ID NO: 170 - protein, 108 aa, synthetic construct
  REGION 1..108: note = VB 13.2 Humanized VL 3
EIVLTQSPATLSLSPGERATLSCSASSSISSTNLHWYQQKPGQAPRLLIYGTSNLASGIPDRFSGSGSGTDFTLTISRLEPEDFAVYYCQQWSRYPLTFGCGTKLEIK

SEQ ID NO: 171 - protein, 108 aa, synthetic construct
  REGION 1..108: note = VB 13.2 Humanized VL 4
EIVLTQSPGTLSLSPGERATLSCSASSSISSTNLHWYQQKPGQAPRLLIYGTSNLASGIPDRFSGSGSGTDFTLTISRLEPEDFAVYYCQQWSRYPLTFGCGTKLEIK

SEQ ID NO: 172 - protein, 108 aa, synthetic construct
  REGION 1..108: note = VB 13.2 Humanized VL 5
EIQLTQSPSSLSASVGDRVTITCRASSSISSTNLHWYQQKPGKAPKPWIYGTSNLASGVPSRFSGSGSGTSYSLTISSLQPEDFATYYCQQWSRYPLTFGCGTKLEIK

SEQ ID NO: 173 - protein, 108 aa, synthetic construct
  REGION 1..108: note = VB 13.2 Humanized VL 6
AIQLTQSPSSLSASVGDRVTITCRASSSISSTNLHWYQQKPGKAPKPLIYGTSNLASGVPSRFSGSGSGTDYTLTISSLQPEDFATYYCQQWSRYPLTFGCGTKLEIK

SEQ ID NO: 174 - protein, 108 aa, synthetic construct
  REGION 1..108: note = VB 13.2 Humanized VL 7
EIVLTQSPATLSLSPGERATLSCRASSSISSTNLHWYQQKPGQAPRPWIYGTSNLASGVPARFSGSGSGTSYSLTISSLEPEDFATYYCQQWSRYPLTFGCGTKLEIK

SEQ ID NO: 175 - protein, 108 aa, synthetic construct
  REGION 1..108: note = VB 13.2 Humanized VL 8
EIVLTQSPATLSLSPGERATLSCRASSSISSTNLHWYQQKPGQAPRPLIYGTSNRATGIPARFSGSGSGTDYTLTISSLEPEDFAVYYCQQWSRYPLTFGCGTKLEIK

SEQ ID NO: 176 - protein, 111 aa, synthetic construct
  REGION 1..111: note = VB 14 Humanized VL 1
DIVLTQSPASLAVSPGERATISCRASESVDSYGNSFMHWYQQKPGQPPKLLIYRASNLESGVPDRFSGSGSGTDFTLTISRVEADDVAVYYCQQSNEDPYTFGCGTKLEIK

SEQ ID NO: 177 - protein, 111 aa, synthetic construct
  REGION 1..111: note = VB 14 Humanized VL 2
DIVLTQSPASLAVSPGERATISCRASESVDSYGNSFMHWYQQKPGQPPKLLIYRASNLESGVPDRFSGSGSGTDFTLTISRVEAEDVAVYYCQQSNEDPYTFGCGTKLEIK

SEQ ID NO: 178 - protein, 111 aa, synthetic construct
  REGION 1..111: note = VB 14 Humanized VL 3
DIVMTQSPDSLAVSLGERATINCRASESVDSYGNSFMHWYQQKPGQPPKLLIYRASNLESGVPDRFSGSGSGTDFTLTISSLQAEDVAVYYCQQSNEDPYTFGCGTKLEIK

SEQ ID NO: 179 - protein, 111 aa, synthetic construct
  REGION 1..111: note = VB 14 Humanized VL 4
EIVLTQSPATLSLSPGERATLSCRASESVDSYGNSFMHWYQQKPGQPPRLLIYRASNLESGVPDRFSGSGSGTDFTLTISRLEAEDVAVYYCQQSNEDPYTFGCGTKLEIK

SEQ ID NO: 180 - protein, 111 aa, synthetic construct
  REGION 1..111: note = VB 14 Humanized VL 5
EIVLTQSPATLSLSPGERATLSCRASESVDSYGNSFMHWYQQKPGQPPRLLIYRASNLESGVPDRFSGSGSGTDFTLTISSLEAEDFAVYYCQQSNEDPYTFGCGTKLEIK

SEQ ID NO: 181 - protein, 111 aa, synthetic construct
  REGION 1..111: note = VB 14 Humanized VL 6
EIVLTQSPGTLSLSPGERATLSCRASESVDSYGNSFMHWYQQKPGQPPRLLIYRASNLESGVPDRFSGSGSGTDFTLTISRLEAEDVAVYYCQQSNEDPYTFGCGTKLEIK

SEQ ID NO: 182 - protein, 109 aa, synthetic construct
  REGION 1..109: note = VB 22 Humanized VL 1
QAVVTQEPSLTVSPGGTVTLTCRSSTGAVTTSNYANWFQQKPGHAPRTLIYGTNNRAPGVPARFSGSLLGDKAALTITGAQTEDEADYFCALWYSNHWVFGCGTKLTVL

SEQ ID NO: 183 - protein, 109 aa, synthetic construct
  REGION 1..109: note = VB 22 Humanized VL 2
QAVVTQEPSLTVSPGGTVTLTCRSSTGAVTTSNYANWFQQKPGQAPRTLIYGTNNRAPWTPARFSGSLLGGKAALTLSGAQPEDEAEYYCALWYSNHWVFGCGTKLTVL

SEQ ID NO: 184 - protein, 109 aa, synthetic construct
  REGION 1..109: note = VB 22 Humanized VL 3
QTVVTQEPSLTVSPGGTVTLTCRSSTGAVTTSNYANWFQQKPGQAPRALIYGTNNRAPWTPARFSGSLLGGKAALTLSGVQPEDEAEYYCALWYSNHWVFGCGTKLTVL

SEQ ID NO: 185 - protein, 109 aa, synthetic construct
  REGION 1..109: note = VB 22 Humanized VL 4
QTVVTQEPSFSVSPGGTVTLTCRSSTGAVTTSNYANWYQQTPGQAPRTLIYGTNNRAPGVPDRFSGSILGNKAALTITGAQADDESDYYCALWYSNHWVFGCGTKLTVL

SEQ ID NO: 186 - protein, 107 aa, synthetic construct
  REGION 1..107: note = VB 11 Humanized VL 1
DIQMTQSPSSLSASVGDRVTITCKASQDINSYLSWFQQKPGKAPKLLIYRANRLVDGVPSRFSGSGSGTDYTLTISSLQPEDFATYYCLQYDEFPFTFGCGTRLEIK

SEQ ID NO: 187 - protein, 107 aa, synthetic construct
  REGION 1..107: note = VB 11 Humanized VL 2
DIQMTQSPSSLSASVGDRVTITCKASQDINSYLSWFQQKPGKAPKSLIYRANRLVDGVPSRFSGSGSGTDFTLTISSLQPEDFATYYCLQYDEFPFTFGCGTRLEIK

SEQ ID NO: 188 - protein, 107 aa, synthetic construct
  REGION 1..107: note = VB 11 Humanized VL 3
DIQMTQSPSSLSASVGDRVTITCKASQDINSYLSWYQQKPGKAPKLLIYRANRLVDGVPSRFSGSGSGTDFTFTISSLQPEDIATYYCLQYDEFPFTFGCGTRLEIK

SEQ ID NO: 189 - protein, 107 aa, synthetic construct
  REGION 1..107: note = VB 11 Humanized VL 4
DIQMTQSPSSLSASVGDRVTITCKASQDINSYLSWYQQKPGKAPKLLLYRANRLVDGVPSRFSGSGSGTDYTLTISSLQPEDFATYYCLQYDEFPFTFGCGTRLEIK

SEQ ID NO: 190 - protein, 107 aa, synthetic construct
  REGION 1..107: note = VB 1 Humanized VL 1
DVQMTQSPSTLSASVGESVTITCKASKSINKYLAWYQQKPGKAPKLLIYDGSTLQSGVPSRFSGSGSGTDFTLTISGLQPEDFATYYCQQHNEYPPTFGCGTKLEIK

SEQ ID NO: 191 - protein, 107 aa, synthetic construct
  REGION 1..107: note = VB 1 Humanized VL 2
DIQMTQSPSSLSASVGDRVTITCKASKSINKYLAWYQQKPGKAPKLLIYDGSTLQSGVPSRFSGSGSGTDFTLTISSLQPEDFATYYCQQHNEYPPTFGCGTKLEIK

SEQ ID NO: 192 - protein, 107 aa, synthetic construct
  REGION 1..107: note = VB 1 Humanized VL 3
DIQMTQSPSTLSASVGDRVTITCKASKSINKYLAWYQQKPGKAPKLLIYDGSTLQSGVPSRFSGSGSGTEFTLTISSLQPDDFATYYCQQHNEYPPTFGCGTKLEIK

SEQ ID NO: 193 - protein, 107 aa, synthetic construct
  REGION 1..107: note = VB 1 Humanized VL 4
DIQLTQSPSFLSASVGDRVTITCKASKSINKYLAWYQQKPGKAPKLLIYDGSTLQSGVPSRFSGSGSGTEFTLTISSLQPEDFATYYCQQHNEYPPTFGCGTKLEIK

SEQ ID NO: 194 - protein, 107 aa, synthetic construct
  REGION 1..107: note = VB 1 Humanized VL 5
DIQMTQSPSSLSASVGDRVTITCKASKSINKYLAWFQQKPGKAPKSLIYDGSTLQSGVPSRFSGSGSGTDFTLTISSLQPEDFATYYCQQHNEYPPTFGCGTKLEIK

SEQ ID NO: 195 - protein, 108 aa, synthetic construct
  REGION 1..108: note = VB 12 Humanized VL 1
EIVLTQSPSFLSASPGEKVTITCSVSSSVSYMEIWYQQKPGQAPKLLIYDTSKLASGVPARFSGSGSGTDFTLTISSLEAEDFATYYCQQWSSNPQYTFGCGTKLEIK

SEQ ID NO: 196 - protein, 108 aa, synthetic construct
  REGION 1..108: note = VB 12 Humanized VL 2
AIQLTQSPSSLSASVGDRVTITCSVSSSVSYMEIWYQQKPGKAPKLLIYDTSKLASGVPSRFSGSGSGTDFTLTISSLQPEDFATYYCQQWSSNPQYTFGCGTKLEIK

SEQ ID NO: 197 - protein, 108 aa, synthetic construct
  REGION 1..108: note = VB 12 Humanized VL 3
EIVLTQSPATLSLSPGERATLSCSVSSSVSYMEIWYQQKPGQAPRLLIYDTSKLASGIPARFSGSGSGTDFTLTISSLEPEDFAVYYCQQWSSNPQYTFGCGTKLEIK

SEQ ID NO: 198 - protein, 108 aa, synthetic construct
  REGION 1..108: note = VB 12 Humanized VL 4
DIQMTQSPSSLSASVGDRVTITCSVSSSVSYMEIWYQQKPGKAPKLLIYDTSKLASGVPSRFSGSGSGTDFTLTISSLQPEDFATYYCQQWSSNPQYTFGCGTKLEIK

SEQ ID NO: 199 - protein, 108 aa, synthetic construct
  REGION 1..108: note = VB 12 Humanized VL 5
DIQLTQSPSFLSASVGDRVTITCSVSSSVSYMEIWYQQKPGKAPKLLIYDTSKLASGVPSRFSGSGSGTEFTLTISSLQPEDFATYYCQQWSSNPQYTFGCGTKLEIK

SEQ ID NO: 200 - protein, 106 aa, synthetic construct
  REGION 1..106: note = VB 8 Humanized VL 1
EIVLTQSPAILSVSLGEKVTLSCRASSSVNYIYWYQQKPGKAPKLLIYYTSNLAPGVPTRFSGSGSGTDFSLTISSLQPEDAATYYCQQFTSSPFTFGCGTKLEIK

SEQ ID NO: 201 - protein, 106 aa, synthetic construct
  REGION 1..106: note = VB 8 Humanized VL 2
EIVLTQSPATLSLSPGERATLSCRASSSVNYIYWYQQKPGQAPRLLIYYTSNLAPGIPARFSGSGSGTDFTLTISSLEPEDFAVYYCQQFTSSPFTFGCGTKLEIK

SEQ ID NO: 202 - protein, 106 aa, synthetic construct
  REGION 1..106: note = VB 8 Humanized VL 3
DIQLTQSPSSLSASVGDRVTITCRASSSVNYIYWYQQKPGKAPKLLIYYTSNLAPGVPSRFSGSGSGTDFTLTISSLQPEDFATYYCQQFTSSPFTFGCGTKLEIK

SEQ ID NO: 203 - protein, 106 aa, synthetic construct
  REGION 1..106: note = VB 13.1 Humanized VL 1
EIVLTQSPAILSASPGEKVTLTCSAGSSVSFMEIWYQQKPGQSPKLLIYDTSKLASGVPGRFSGSGSGTDFSLTISRLEPDDAAYYYLQGSGFPLTFGCGTKLEIK

SEQ ID NO: 204 - protein, 107 aa, synthetic construct
  REGION 1..107: note = VB 13.1 Humanized VL 2
EIVLTQSPATLSLSPGERATLSCSAGSSVSFMEIWYQQKPGQAPRLLIYDTSKLASGIPARFSGSGSGTDFTLTISSLEPEDFAVYYCLQGSGFPLTFGCGTKLEIK

SEQ ID NO: 205 - protein, 107 aa, synthetic construct
  REGION 1..107: note = VB 13.1 Humanized VL 3
EIVLTQSPATLSLSPGERATLSCSAGSSVSFMEIWYQQKPGQAPRLLIYDTSKLASGIPDRFSGSGSGTDFTLTISRLEPEDFAVYYCLQGSGFPLTFGCGTKLEIK

SEQ ID NO: 206 - protein, 106 aa, synthetic construct
  REGION 1..106: note = VB 5.1 Humanized VL 1
EIVLTQSPAILAVSLGEKVTITCSASSSVSYMHWYQQKPGKAPKLLIYEISKLASGVPARFSGSGSGTSFSLTISSLQAEDAATYYCQQWNYPLLTFGCGTKLEIK

SEQ ID NO: 207 - protein, 106 aa, synthetic construct
  REGION 1..106: note = VB 5.1 Humanized VL 2
DIQLTQSPSFLSASVGDRVTITCSASSSVSYMHWYQQKPGKAPKLLIYEISKLASGVPSRFSGSGSGTEFTLTISSLQPEDFATYYCQQWNYPLLTFGCGTKLEIK

SEQ ID NO: 208 - protein, 106 aa, synthetic construct
  REGION 1..106: note = VB 5.1 Humanized VL 3
DIQLTQSPSSLSASVGDRVTITCSASSSVSYMHWYQQKPGKAPKLLIYEISKLASGVPSRFSGSGSGTDFTLTISSLQPEDFATYYCQQWNYPLLTFGCGTKLEIK

SEQ ID NO: 209 - protein, 107 aa, synthetic construct
  REGION 1..107: note = VB 9 Humanized VL 1
ETVMTQSPASLSVAPGEKVTITCISSTDIDDDMNWYQQKPGEPPKLLISEGNTLRPGVPSRFSGSGYGTDFTFTINNLQSEDVADYYCLQSDNMPLTFGCGTKLEIK

SEQ ID NO: 210 - protein, 107 aa, synthetic construct
  REGION 1..107: note = VB 9 Humanized VL 2
ETTLTQSPAFMSATPGDKVNISCISSTDIDDDMNWYQQKPGEAPIFIIQEGNTLRPGIPPRFSGSGYGTDFTLTINNIESEDAAYYFCLQSDNMPLTFGCGTKLEIK

SEQ ID NO: 211 - protein, 107 aa, synthetic construct
  REGION 1..107: note = VB 9 Humanized VL 3
DIQMTQSPSSLSASVGDRVTITCISSTDIDDDMNWYQQKPGKAPKLLIYEGNTLRPGVPSRFSGSGSGTDFTFTISSLQPEDIATYYCLQSDNMPLTFGCGTKLEIK

SEQ ID NO: 212 - protein, 107 aa, synthetic construct
  REGION 1..107: note = VB 9 Humanized VL 4
DVVMTQSPAFLSVTPGEKVTITCISSTDIDDDMNWYQQKPGQAPKLLIKEGNTLRPGVPSRFSGSGSGTDFTLTISSLEAEDAATYYCLQSDNMPLTFGCGTKLEIK

SEQ ID NO: 213 - protein, 112 aa, synthetic construct
  REGION 1..112: note = VB 17 Humanized VL 1
DVVMTQTPLSLPVTLGQPASISCRSSQSLVHSNGNTYLHWYLQKPGQSPQLLIYKVSNRFSGVPDRFSGSGSGTDFTLKISRVEAEDVGVYYCSQSTHVPFTFGCGTKLEIK

SEQ ID NO: 214 - protein, 112 aa, synthetic construct
  REGION 1..112: note = VB 17 Humanized VL 2
DVVMTQSPLSLPVTLGQPASISCRSSQSLVHSNGNTYLHWFQQRPGQSPRRLIYKVSNRFSGVPDRFSGSGSGTDFTLKISRVEAEDVGVYYCSQSTHVPFTFGCGTKLEIK

SEQ ID NO: 215 - protein, 112 aa, synthetic construct
  REGION 1..112: note = VB 17 Humanized VL 3
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQSTHVPFTFGCGTKLEIK

SEQ ID NO: 216 - protein, 106 aa, synthetic construct
  REGION 1..106: note = VB 3 Humanized VL 1
EIVLTQSPAILSASLGERITLTCSANSTVGYIEIWYQQKPGQAPKLLIYTTSNLASGVPSRFSGSGSGTDFSLTISSLEAEDFAVYFCHQWSFYPTFGCGTKLEIK

SEQ ID NO: 217 - protein, 106 aa, synthetic construct
  REGION 1..106: note = VB 3 Humanized VL 2
EIVLTQSPATLSLSPGERATLSCSANSTVGYIEIWYQQKPGQAPRLLIYTTSNLASGIPARFSGSGSGTDFTLTISSLEPEDFAVYYCHQWSFYPTFGCGTKLEIK

SEQ ID NO: 218 - protein, 106 aa, synthetic construct
  REGION 1..106: note = VB 3 Humanized VL 3
EIVLTQSPATLSLSPGERATLSCSANSTVGYIEIWYQQKPGQAPRLLIYTTSNLASGIPARFSGSGPGTDFTLTISSLEPEDFAVYYCHQWSFYPTFGCGTKLEIK

SEQ ID NO: 219 - protein, 111 aa, synthetic construct
  REGION 1..111: note = VB 7.1 Humanized VL 1
DIVLTQSPVSLPVSPGERATISCRASKSVSTSGYSYMHWYQQKPGQPPKLLIYLASNLESGVPDRFSGSGSGTDFTLTISRVEAEDVAVYYCQHSRDLPWTFGCGTKLEIK

SEQ ID NO: 220 - protein, 111 aa, synthetic construct
  REGION 1..111: note = VB 7.1 Humanized VL 2
DIVLTQSPASLAVSPGQRATITCRASKSVSTSGYSYMHWYQQKPGQPPKLLIYLASNLESGVPARFSGSGSGTDFTLTINPVEANDTANYYCQHSRDLPWTFGCGTKVEIK

SEQ ID NO: 221 - protein, 111 aa, synthetic construct
  REGION 1..111: note = VB 7.1 Humanized VL 3
DIVMTQSPLSLPVTPGEPASISCRASKSVSTSGYSYMHWYLQKPGQSPQLLIYLASNLESGVPDRFSGSGSGTDFTLKISRVEAEDVGVYYCQHSRDLPWTFGCGTKVEIK

SEQ ID NO: 222 - protein, 111 aa, synthetic construct
  REGION 1..111: note = VB 7.1 Humanized VL 4
EIVLTQSPGTLSLSPGERATLSCRASKSVSTSGYSYMHWYQQKPGQPPRLLIYLASNLESGVPDRFSGSGSGTDFTLTISRLEAEDVAVYYCQHSRDLPWTFGCGTKVEIK

SEQ ID NO: 223 - protein, 111 aa, synthetic construct
  REGION 1..111: note = VB 7.1 Humanized VL 5
EIVLTQSPATLSLSPGERATLSCRASKSVSTSGYSYMHWYQQKPGQPPRLLIYLASNLESGVPDRFSGSGSGTDFTLTISRLEAEDVAVYYCQHSRDLPWTFGCGTKVEIK

SEQ ID NO: 224 - protein, 111 aa, synthetic construct
  REGION 1..111: note = VB 7.1 Humanized VL 6
DIVMTQSPDSLAVSLGERATINCRASKSVSTSGYSYMHWYQQKPGQPPKLLIYLASNLESGVPDRFSGSGSGTDFTLTISSLQAEDVAVYYCQHSRDLPWTFGCGTKVEIK

SEQ ID NO: 225 - protein, 111 aa, synthetic construct
  REGION 1..111: note = VB 5.3 Humanized VL 1
DIVLTQSPASLAVSPGERATISCRASESVDSSGNSFMHWYQQKPGQPPQLLIYRASNLESGVPDRFSGSGSGTDFTLTISRVEADDVAVYYCQQSFDDPFTFGCGTKLEIK

SEQ ID NO: 226 - protein, 111 aa, synthetic construct
  REGION 1..111: note = VB 5.3 Humanized VL 2
DIVLTQSPASLAVSPGQRATITCRASESVDSSGNSFMHWYQQKPGQPPKLLIYRASNLESGVPARFSGSGSGTDFTLTINPVEANDTANYYCQQSFDDPFTFGCGTKLEIK

SEQ ID NO: 227 - protein, 111 aa, synthetic construct
  REGION 1..111: note = VB 5.3 Humanized VL 3
EIVLTQSPATLSLSPGERATLSCRASESVDSSGNSFMHWYQQKPGQAPRLLIYRASNLESGIPARFSGSGSGTDFTLTISSLEPEDFAVYYCQQSFDDPFTFGCGTKLEIK

SEQ ID NO: 228 - protein, 119 aa, synthetic construct
  REGION 1..119: note = anti-VB 2 Humanized VH 1
QVQLVQSGAEVKKPGASVKVSCKASGFNIKSAYMHWVRQAPGQGLEWLGRIDPATGKTKYAPKFQGRATITADTSSNTAYMELSSLRSEDTAVYYCTRSLNWDYGLDYWGQGTTVTVSS

SEQ ID NO: 229 - protein, 119 aa, synthetic construct
  REGION 1..119: note = anti-VB 2 Humanized VH 2
QVQLVQSGAEVKKPGASVKVSCKASGFNIKSAYMHWVRQAPGQGLEWMGRIDPATGKTKYAPKFQGRVTMTADTSTSTAYMELSSLRSEDTAVYYCTRSLNWDYGLDYWGQGTTVTVSS

SEQ ID NO: 230 - protein, 119 aa, synthetic construct
  REGION 1..119: note = anti-VB 2 Humanized VH 3
QVQLQQSGPGLVKPSETLSLTCTASGFNIKSAYMHWVRQPPGKGLEWLGRIDPATGKTKYAPSLKSRATISADTSSNTASLKLSSVTAADTAVYYCTRSLNWDYGLDYWGQGTTVTVSS

SEQ ID NO: 231 - protein, 119 aa, synthetic construct
  REGION 1..119: note = anti-VB 2 Humanized VH 4
QVQLQESGPGLVKPSETLSLTCTASGFNIKSAYMHWIRQPPGKGLEWIGRIDPATGKTKYAPSLKSRVTISADTSKNQASLKLSSVTAADTAVYYCTRSLNWDYGLDYWGQGTTVTVSS

SEQ ID NO: 232 - protein, 119 aa, synthetic construct
  REGION 1..119: note = anti-VB 2 Humanized VH 5
EVQLVQSGADLVRPGASLKLSCTASGFNIKSAYMHWVRQAPGQGPEWLGRIDPATGKTKYAPKFQGRVTITADTSSNTAYLQLSSLTSEDTAIYYCTRSLNWDYGLDYWGQGTLVTVSS

SEQ ID NO: 233 - protein, 119 aa, synthetic construct
  REGION 1..119: note = anti-VB 2 Humanized VH 6
QVQLVQSGAEVKKPGASVKVSCKASGFNIKSAYMHWVRQAPGQGLEWMGLVDPATGKTSYAQKFQGRVTITRDTSMSTAYMELSSLRSEDTAMYYCVRSLNWDYGLDYWGQGTLVTVSS

SEQ ID NO: 234 - protein, 119 aa, synthetic construct
  REGION 1..119: note = anti-VB 2 Humanized VH 7
QVQLVQSGAEVKKPGASVKVSCKASGFNIKSAYMHWVRQAPGQGLEWMGRIDPATGKTNYAQKFQGRVTMTRDTSISTAYMELSRLRSDDTAVYYCARSLNWDYGLDYWGQGTLVTVSS

SEQ ID NO: 235 - protein, 119 aa, synthetic construct
  REGION 1..119: note = anti-VB 2 Humanized VH 8
QVQLVQSGAEVKKPGASVKVSCKASGFNIKSAAMHWVRQAPGQRLEWMGWIDPATGKTKYSQKFQGRVTITRDTSASTAYMELSSLRSEDTAVYYCARSLNWDYGLDYWGQGTLVTVSS

SEQ ID NO: 236 - protein, 117 aa, synthetic construct
  REGION 1..117: note = anti-VB 7.2 Humanized VH 1
QVHLVQSGAEVKRPGASVKVSCKASGYIFTDYYMHWVRQAPGQGLEWMGWINPSSGYTTYAQKFQDRVTLTADKSSSTAYMELSSLTSEDTAVYYCARSLQLGRDYWGQGTLVTVSS

SEQ ID NO: 237 - protein, 117 aa, synthetic construct
  REGION 1..117: note = anti-VB 7.2 Humanized VH 2
QVQLVQSGAEVKKPGASVKVSCKASGYIFTDYYMHWVRQAPGQGLEWMGWINPSSGYTNYAQKFQGRVTMTRDTSISTAYMELSRLRSDDTAVYYCARSLQLGRDYWGQGTLVTVSS

SEQ ID NO: 238 - protein, 117 aa, synthetic construct
  REGION 1..117: note = anti-VB 7.2 Humanized VH 3
QVQLVQSGAEVKKPGASVKVSCKASGYIFTDYYMHWVRQAPGQGLEWMGIINPSSGYTSYAQKFQGRVTMTRDTSTSTVYMELSSLRSEDTAVYYCARSLQLGRDYWGQGTLVTVSS

SEQ ID NO: 239 - protein, 120 aa, synthetic construct
  REGION 1..120: note = anti-VB 4 Humanized VH 1
QVQLVQSGAEVKKPGASVKVSCKASGYTFTSYYIYWVRQAPGQGLEWLGWIYPGNGGTYYSEKFQGRVTFTADTSTNTAYMELSSLTSEDTAVYFCARGSGDRYNSLAYWGQGTLVTVSS

SEQ ID NO: 240 - protein, 120 aa, synthetic construct
  REGION 1..120: note = anti-VB 4 Humanized VH 2
QVQLVQSGAEVKKPGASVKVSCKASGYTFTSYAMHWVRQAPGQRLEWMGWIYPGNGGTKYSQKFQGRVTITRDTSASTAYMELSSLRSEDTAVYYCARGSGDRYNSLAYWGQGTLVTVSS

SEQ ID NO: 241 - protein, 120 aa, synthetic construct
  REGION 1..120: note = anti-VB 4 Humanized VH 3
QVQLVQSGAEVKKPGASVKVSCKASGYTFTSYYMHWVRQAPGQGLEWMGIIYPGNGGTSYAQKFQGRVTMTRDTSTSTVYMELSSLRSEDTAVYYCARGSGDRYNSLAYWGQGTLVTVSS

SEQ ID NO: 242 - protein, 120 aa, synthetic construct
  REGION 1..120: note = anti-VB 4 Humanized VH 4
QVQLVQSGAEVKKPGASVKVSCKASGYTFTSYGISWVRQAPGQGLEWMGWIYPGNGGTNYAQKLQGRVTMTTDTSTSTAYMELRSLRSDDTAVYYCARGSGDRYNSLAYWGQGTLVTVSS

SEQ ID NO: 243 - protein, 120 aa, synthetic construct
  REGION 1..120: note = anti-VB 4 Humanized VH 5
QVQLVQSGAEVKKPGASVKVSCKASGYTFTSYYMHWVRQAPGQGLEWMGWIYPGNGGTNYAQKFQGRVTMTRDTSISTAYMELSRLRSDDTAVYYCARGSGDRYNSLAYWGQGTLVTVSS

SEQ ID NO: 244 - protein, 120 aa, synthetic construct
  REGION 1..120: note = anti-VB 13.2 Humanized VH 1
QVQLVQSGPEVKKPGASVKVSCKASGYTFTDYYIHWLRQAPGKGLEWIGWVNTNNGDTNYAQRFKGRVSLTRDKSTSTAYMELNSLTSEDTAVYYCARALYTGSYWFAYWGQGTLVTVSS

SEQ ID NO: 245 - protein, 120 aa, synthetic construct
  REGION 1..120: note = anti-VB 13.2 Humanized VH 2
QVQLVQSGAEVKKPGASVKVSCKASGYTFTDYYMHWVRQAPGQGLEWMGWINTNNGDTNYAQKFQGRVTMTRDTSISTAYMELSRLRSDDTAVYYCARALYTGSYWFAYWGQGTLVTVSS

SEQ ID NO: 246 - protein, 120 aa, synthetic construct
  REGION 1..120: note = anti-VB 13.2 Humanized VH 3
QVQLVQSGAEVKKPGASVKVSCKASGYTFTDYYMHWVRQAPGQGLEWMGIINTNNGDTSYAQKFQGRVTMTRDTSTSTVYMELSSLRSEDTAVYYCARALYTGSYWFAYWGQGTLVTVSS

SEQ ID NO: 247 - protein, 120 aa, synthetic construct
  REGION 1..120: note = anti-VB 13.2 Humanized VH 4
QVQLVQSGAEVKKPGASVKVSCKASGYTFTDYYIHWLRQAPGQGLEWIGIVNTNNGDTNYNQKFQGRATLTVDKSSSTAYMELSSLRSEDTAVYYCARALYTGSYWFAYWGQGTLVTVSS

SEQ ID NO: 248 - protein, 120 aa, synthetic construct
  REGION 1..120: note = anti-VB 13.2 Humanized VH 5
QVQLVQSGAEVKKPGASVKVSCKASGYTFTDYYIHWVRQAPGQGLEWMGIVNTNNGDTNYNQKFQGRVTMTVDKSTSTAYMELSSLRSEDTAVYYCARALYTGSYWFAYWGQGTLVTVSS

SEQ ID NO: 249 - protein, 120 aa, synthetic construct
  REGION 1..120: note = anti-VB 13.2 Humanized VH 6
QVQLQQSGPGLVKPSETLSLTCTASGYTFTDYYIHWLRQPPGKGLEWIGIVNTNNGDTNYNQRLKSRATLSVDKSSSTASLKLSSVTAADTAVYYCARALYTGSYWFAYWGQGTLVTVSS

SEQ ID NO: 250 - protein, 120 aa, synthetic construct
  REGION 1..120: note = anti-VB 13.2 Humanized VH 7
QVQLQESGPGLVKPSETLSLTCTASGYTFTDYYIHWIRQPPGKGLEWIGIVNTNNGDTNYNQRLKSRVTISVDKSKNQASLKLSSVTAADTAVYYCARALYTGSYWFAYWGQGTLVTVSS

SEQ ID NO: 251 - protein, 118 aa, synthetic construct
  REGION 1..118: note = anti-VB 14 Humanized VH 1
QVQLVQSGAEVKKPGASVKLSCKASGFKTEDTYMHWVRQAPGQGLEWIGRIDPANGNTKYAPKFQDRVTITADTSANTAYLELSSLRSEDTAVYYCARGSYYYAMDYWGQGTTVTVSS

SEQ ID NO: 252 - protein, 118 aa, synthetic construct
  REGION 1..118: note = anti-VB 14 Humanized VH 2
QVQLVQSGAEVKKPGASVKVSCKASGFKTEDTAMHWVRQAPGQRLEWMGWIDPANGNTKYSQKFQGRVTITRDTSASTAYMELSSLRSEDTAVYYCARGSYYYAMDYWGQGTTVTVSS

SEQ ID NO: 253 - protein, 118 aa, synthetic construct
  REGION 1..118: note = anti-VB 14 Humanized VH 3
QVQLVQSGAEVKKPGASVKVSCKASGFKTEDTYMHWVRQAPGQGLEWMGRIDPANGNTNYAQKFQGRVTMTRDTSISTAYMELSRLRSDDTAVYYCARGSYYYAMDYWGQGTTVTVSS

SEQ ID NO: 254 - protein, 118 aa, synthetic construct
  REGION 1..118: note = anti-VB 14 Humanized VH 4
QVQLVQSGAEVKKPGASVKVSCKASGFKTEDTYMHWVRQAPGQGLEWMGIIDPANGNTSYAQKFQGRVTMTRDTSTSTVYMELSSLRSEDTAVYYCARGSYYYAMDYWGQGTTVTVSS

SEQ ID NO: 255 - protein, 116 aa, synthetic construct
  REGION 1..116: note = anti-VB 22 Humanized VH 1
EVQLVESGGGLVQPGGSLRLSCAASGFDFSRYWMNWVRQAPGKGLEWVSYINSDSNTINYADSVKGRFTISRDNAKNSLYLQMNSLRAEDTALYYCARGGLLRDVWGQGTTVTVSS

SEQ ID NO: 256 - protein, 116 aa, synthetic construct
  REGION 1..116: note = anti-VB 22 Humanized VH 2
EVQLVESGGGLVQPGGSLRLSCAASGFDFSRYSMNWVRQAPGKGLEWVSYINSDSNTIYYADSVKGRFTISRDNAKNSLYLQMNSLRAEDTAVYYCARGGLLRDVWGQGTMVTVSS

SEQ ID NO: 257 - protein, 116 aa, synthetic construct
  REGION 1..116: note = anti-VB 22 Humanized VH 3
EVQLVESGGGLVKPGGSLRLSCAASGFDFSRYSMNWVRQAPGKGLEWVSSINSDSNTIYYADSVKGRFTISRDNAKNSLYLQMNSLRAEDTAVYYCARGGLLRDVWGQGTMVTVSS

SEQ ID NO: 258 - protein, 116 aa, synthetic construct
  REGION 1..116: note = anti-VB 11 Humanized VH 1
QVQLVQSGPEVKRPGASVKISCKASGYRFTDSAMHWVRQAPGQGLEWMGWISSYNGNTNYAQKFQGRVTMTVDTSTSTAYMELSRLTSEDTAIYYCARSRDAMDYWGQGTLVTVSS

SEQ ID NO: 259 - protein, 116 aa, synthetic construct
  REGION 1..116: note = anti-VB 11 Humanized VH 2
QVQLVQSGAEVKKPGASVKVSCKASGYRFTDSGISWVRQAPGQGLEWMGWISSYNGNTNYAQKLQGRVTMTTDTSTSTAYMELRSLRSDDTAVYYCARSRDAMDYWGQGTLVTVSS

SEQ ID NO: 260 - protein, 116 aa, synthetic construct
  REGION 1..116: note = anti-VB 11 Humanized VH 3
QVQLVQSGAEVKKPGASVKVSCKASGYRFTDSYMHWVRQAPGQGLEWMGWISSYNGNTNYAQKFQGRVTMTRDTSISTAYMELSRLRSDDTAVYYCARSRDAMDYWGQGTLVTVSS

SEQ ID NO: 261 - protein, 121 aa, synthetic construct
  REGION 1..121: note = anti-VB 1 Humanized VH 1
QVQLVQSGSELREPGASVKISCKASGYTFTDYYVHWVRQAPGKGLEWMGWINTYTGTPTYAQDFTGRFVFSLDTSASTAYLQISNLKAEDTAVYFCARSWRRGIRGIGFDWGQGTLVTVSS

SEQ ID NO: 262 - protein, 121 aa, synthetic construct
  REGION 1..121: note = anti-VB 1 Humanized VH 2
QVQLVQSGSELKKPGASVKVSCKASGYTFTDYAMNWVRQAPGQGLEWMGWINTYTGTPTYAQGFTGRFVFSLDTSVSTAYLQISSLKAEDTAVYYCARSWRRGIRGIGFDWGQGTLVTVSS

SEQ ID NO: 263 - protein, 117 aa, synthetic construct
  REGION 1..117: note = anti-VB 12 Humanized VH 1
EVQLVESGGGLVKPGGSLRLSCAASGFTFRSYGMSWVRQAPGKGLEWVASISSGGSYTYYADSVKGRFTISRDNAKNSLYLQMSSLRAEDTAIYYCARHGGNFFDYWGQGTLVTVSS

SEQ ID NO: 264 - protein, 117 aa, synthetic construct
  REGION 1..117: note = anti-VB 12 Humanized VH 2
EVQLVESGGGLVKPGGSLRLSCAASGFTFRSYSMNWVRQAPGKGLEWVSSISSGGSYIYYADSVKGRFTISRDNAKNSLYLQMNSLRAEDTAVYYCARHGGNFFDYWGQGTLVTVSS

SEQ ID NO: 265 - protein, 117 aa, synthetic construct
  REGION 1..117: note = anti-VB 12 Humanized VH 3
EVQLVESGGGLVQPGGSLRLSCAASGFTFRSYEMNWVRQAPGKGLEWVSYISSGGSYIYYADSVKGRFTISRDNAKNSLYLQMNSLRAEDTAVYYCARHGGNFFDYWGQGTLVTVSS

SEQ ID NO: 266 - protein, 117 aa, synthetic construct
  REGION 1..117: note = anti-VB 12 Humanized VH 4
QVQLVESGGGLVKPGGSLRLSCAASGFTFRSYYMSWIRQAPGKGLEWVSYISSGGSYIYYADSVKGRFTISRDNAKNSLYLQMNSLRAEDTAVYYCARHGGNFFDYWGQGTLVTVSS

SEQ ID NO: 267 - protein, 117 aa, synthetic construct
  REGION 1..117: note = anti-VB 8 Humanized VH 1
EVQLVESGGGLVQPGGSLRLSCAASGFTFSNFGMHWVRQAPGKGLEWVAYISSGSSTIYYADSVKGRFTISRDNAKNTLFLQMNSLRAEDTAVYYCARRGEGAMDYWGQGTLVTVSS

SEQ ID NO: 268 - protein, 117 aa, synthetic construct
  REGION 1..117: note = anti-VB 8 Humanized VH 2
EVQLVESGGGLVQPGGSLRLSCAASGFTFSNFSMNWVRQAPGKGLEWVSYISSGSSTIYYADSVKGRFTISRDNAKNSLYLQMNSLRAEDTAVYYCARRGEGAMDYWGQGTLVTVSS

SEQ ID NO: 269 - protein, 117 aa, synthetic construct
  REGION 1..117: note = anti-VB 8 Humanized VH 3
EVQLVESGGGLVKPGGSLRLSCAASGFTFSNFSMNWVRQAPGKGLEWVSYISSGSSTIYYADSVKGRFTISRDNAKNSLYLQMNSLRAEDTAVYYCARRGEGAMDYWGQGTLVTVSS

SEQ ID NO: 270 - protein, 121 aa, synthetic construct
  REGION 1..121: note = anti-VB 13.1 Humanized VH 1
EVQLVESGGGLVKPGGSLRLSCAASGFTFRSYAMSWVRQAPGKGLEWVASISNGGDYIYYADSVKGRFTISRDNARNTLYLQMSSLRAEDTAVYYCARPSYYSDPWFFDVWGRGTTVTVSS

SEQ ID NO: 271 - protein, 121 aa, synthetic construct
  REGION 1..121: note = anti-VB 13.1 Humanized VH 2
EVQLVESGGGLVKPGGSLRLSCAASGFTFRSYSMNWVRQAPGKGLEWVSSISNGGDYIYYADSVKGRFTISRDNAKNSLYLQMNSLRAEDTAVYYCARPSYYSDPWFFDVWGRGTLVTVSS

SEQ ID NO: 272 - protein, 121 aa, synthetic construct
  REGION 1..121: note = anti-VB 13.1 Humanized VH 3
EVQLVESGGGLVQPGGSLRLSCAASGFTFRSYAMSWVRQAPGKGLEWVSAISNGGDYTYYADSVKGRFTISRDNSKNTLYLQMNSLRAEDTAVYYCAKPSYYSDPWFFDVWGRGTLVTVSS

SEQ ID NO: 273 - protein, 121 aa, synthetic construct
  REGION 1..121: note = anti-VB 13.1 Humanized VH 4
EVQLVESGGGLVQPGGSLRLSCAASGFTFRSYEMNWVRQAPGKGLEWVSYISNGGDYIYYADSVKGRFTISRDNAKNSLYLQMNSLRAEDTAVYYCARPSYYSDPWFFDVWGRGTLVTVSS

SEQ ID NO: 274 - protein, 118 aa, synthetic construct
  REGION 1..118: note = anti-VB 5.1 Humanized VH 1
QVQLVQSGPELKKPGASVRVSCKASGYTFTDYYIHWVRQAPGRGLEWVGWINPYNGRTGYAQKFKGRVTLTRDKSSSTAYMDLRSLTSEDTAVYYCARWDGSSYFDYWGQGTLVTVSS

SEQ ID NO: 275 - protein, 118 aa, synthetic construct
  REGION 1..118: note = anti-VB 5.1 Humanized VH 2
QVQLVQSGAEVKKPGASVKVSCKASGYTFTDYYMHWVRQAPGQGLEWMGWINPYNGRTNYAQKFQGRVTMTRDTSISTAYMELSRLRSDDTAVYYCARWDGSSYFDYWGQGTLVTVSS

SEQ ID NO: 276 - protein, 118 aa, synthetic construct
  REGION 1..118: note = anti-VB 5.1 Humanized VH 3
QVQLVQSGAEVKKPGASVKVSCKASGYTFTDYYMHWVRQAPGQGLEWMGIINPYNGRTSYAQKFQGRVTMTRDTSTSTVYMELSSLRSEDTAVYYCARWDGSSYFDYWGQGTLVTVSS

SEQ ID NO: 277 - protein, 118 aa, synthetic construct
  REGION 1..118: note = anti-VB 5.1 Humanized VH 4
QVQLVQSGAEVKKPGASVKVSCKASGYTFTDYGISWVRQAPGQGLEWMGWINPYNGRTNYAQKLQGRVTMTTDTSTSTAYMELRSLRSDDTAVYYCARWDGSSYFDYWGQGTLVTVSS

SEQ ID NO: 278 - protein, 118 aa, synthetic construct; identical to SEQ ID NO: 275
  REGION 1..118: note = anti-VB 5.1 Humanized VH 5

SEQ ID NO: 279 - protein, 118 aa, synthetic construct
  REGION 1..118: note = anti-VB 9 Humanized VH 1
QVQLVQSGAELKRPGASVKLSCTASGFNIKNTFMHWVRQAPGQGLEWIGRIDPTNGYTKFAPKFQGRVTLTADTSSNTVYLQLSSLTSEDTAIYYCARDYDAPWFAYWGQGTLVIVSS

SEQ ID NO: 280 - protein, 118 aa, synthetic construct
  REGION 1..118: note = anti-VB 9 Humanized VH 2
QVQLVQSGAEVKKPGASVKVSCKASGFNIKNTYMHWVRQAPGQGLEWMGIIDPTNGYTSYAQKFQGRVTMTRDTSTSTVYMELSSLRSEDTAVYYCARDYDAPWFAYWGQGTLVTVSS

SEQ ID NO: 281 - protein, 118 aa, synthetic construct
  REGION 1..118: note = anti-VB 9 Humanized VH 3
QVQLVQSGAEVKKPGASVKVSCKASGFNIKNTYMHWVRQAPGQGLEWMGRIDPTNGYTNYAQKFQGRVTMTRDTSISTAYMELSRLRSDDTAVYYCARDYDAPWFAYWGQGTLVTVSS

SEQ ID NO: 282 - protein, 118 aa, synthetic construct
  REGION 1..118: note = anti-VB 9 Humanized VH 4
QVQLVQSGAEVKKPGASVKVSCKASGFNIKNTAMHWVRQAPGQRLEWMGWIDPTNGYTKYSQKFQGRVTITRDTSASTAYMELSSLRSEDTAVYYCARDYDAPWFAYWGQGTLVTVSS

SEQ ID NO: 283 - protein, 120 aa, synthetic construct
  REGION 1..120: note = anti-VB 17 Humanized VH 1
QVQLQESGPGLVKPSQTLSLTCSVSGYSITSGYFWNWIRQPPGKGLEWIGYISYDGSTNYNPSLKSRISISVDTSKNQFSLKLNSVTAADTAVYYCASPSPGTGYAVDYWGQGTLVTVSS

SEQ ID NO: 284 - protein, 119 aa, synthetic construct
  REGION 1..119: note = anti-VB 17 Humanized VH 2
QVQLQESGPGLVKPSETLSLTCTVSGYSITSGYWSWIRQPPGKGLEWIGYISYDGSTNYNPSLKSRVTISVDTSKNQFSLKLSSVTAADTAVYYCARPSPGTGYAVDYWGQGTLVTVSS

SEQ ID NO: 285 - protein, 120 aa, synthetic construct
  REGION 1..120: note = anti-VB 17 Humanized VH 3
QVQLQESGPGLVKPSQTLSLTCAVSGYSITSGYYWGWIRQPPGKGLEWIGYISYDGSTYYNPSLKSRVTISVDTSKNQFSLKLSSVTAADTAVYYCARPSPGTGYAVDYWGQGTLVTVSS

SEQ ID NO: 286 - protein, 121 aa, synthetic construct
  REGION 1..121: note = anti-VB 3 Humanized VH 1
QVQLVQSGPEVKKPGASVKISCKASGYTFSGSWMHWVRQAPGKGLEWIGRIYPGDGDTDYSQKFQGRVTLTADKSSSTAYMELSSLTSEDTAVYFCARSGYFNYVPVFDYWGQGTLVTVSS

SEQ ID NO: 287 - protein, 121 aa, synthetic construct
  REGION 1..121: note = anti-VB 3 Humanized VH 2
QVQLVQSGAEVKKPGASVKVSCKASGYTFSGSYMHWVRQAPGQGLEWMGRIYPGDGDTNYAQKFQGRVTMTRDTSISTAYMELSRLRSDDTAVYYCARSGYFNYVPVFDYWGQGTLVTVSS

SEQ ID NO: 288 - skipped

SEQ ID NO: 289 - protein, 121 aa, synthetic construct
  REGION 1..121: note = anti-VB 3 Humanized VH 3
QVQLVQSGAEVKKPGASVKVSCKASGYTFSGSAMHWVRQAPGQRLEWMGWIYPGDGDTKYSQKFQGRVTITRDTSASTAYMELSSLRSEDTAVYYCARSGYFNYVPVFDYWGQGTLVTVSS

SEQ ID NO: 290 - protein, 123 aa, synthetic construct
  REGION 1..123: note = anti-VB 7.1 Humanized VH 1
EVQLVESGGGLVKPGGSLRLSCAASGFTFSDYYMNWVRQTPGKGLEWVASISGGGSYTYYADSVKGRFTISRDNAKNSLYLQMSSLRAEDTAVYFCARERDIYYGNFNAMVYWGQGTLVTVSS

SEQ ID NO: 291 - protein, 123 aa, synthetic construct
  REGION 1..123: note = anti-VB 7.1 Humanized VH 2
EVQLVESGGGLVKPGGSLRLSCAASGFTFSDYYMNWVRQAPGKGLEWVSSISGGGSYIYYADSVKGRFTISRDNAKNSLYLQMNSLRAEDTAVYYCARERDIYYGNFNAMVYWGRGTLVTVSS

SEQ ID NO: 292 - protein, 123 aa, synthetic construct
  REGION 1..123: note = anti-VB 7.1 Humanized VH 3
EVQLVESGGGLVKPGGSLRLSCAASGFTFSDYSMNWVRQAPGKGLEWVSSISGGGSYIYYADSVKGRFTISRDNAKNSLYLQMNSLRAEDTAVYYCARERDIYYGNFNAMVYWGRGTLVTVSS

SEQ ID NO: 293 - protein, 123 aa, synthetic construct
  REGION 1..123: note = anti-VB 7.1 Humanized VH 4
QVQLVESGGGLVKPGGSLRLSCAASGFTFSDYYMSWIRQAPGKGLEWVSYISGGGSYIYYADSVKGRFTISRDNAKNSLYLQMNSLRAEDTAVYYCARERDIYYGNFNAMVYWGQGTLVTVSS

SEQ ID NO: 294 - protein, 119 aa, synthetic construct
  REGION 1..119: note = anti-VB 5.3 Humanized VH 1
QVQLVQSGPELKKPGASVKISCKASGYAFSSSWMHWVRQAPGQGLEWIGRIYPGDGDTKYAQKFQGRVTLTADKSSSTAYMELSSLTSEDTAVYFCARRGTGGWYFDVWGKGTLVTVSS

SEQ ID NO: 295 - protein, 119 aa, synthetic construct
  REGION 1..119: note = anti-VB 5.3 Humanized VH 2
QVQLVQSGAEVKKPGASVKVSCKASGYAFSSSAMHWVRQAPGQRLEWMGWIYPGDGDTKYSQKFQGRVTITRDTSASTAYMELSSLRSEDTAVYYCARRGTGGWYFDVWGRGTLVTVSS

SEQ ID NO: 296 - protein, 111 aa, synthetic construct
  REGION 1..111: note = anti-VB 2 Humanized VL 1
DIVLTQSPDSLAVSLGERATINCKASKSVSILGTHLIHWYQQKPGQPPKLLIYAASNLESGVPDRFSGSGSETVFTLTISSLQAEDVATYFCQQSIEDPWTFGGGTKLEIK

SEQ ID NO: 297 - protein, 111 aa, synthetic construct
  REGION 1..111: note = anti-VB 2 Humanized VL 2
DIVLTQSPDSLAVSLGERATINCKASKSVSILGTHLIHWYQQKPGQPPKLLIYAASNRESGVPDRFSGSGSETDFTLTISSLQAEDVAVYYCQQSIEDPWTFGGGTKLEIK

SEQ ID NO: 298 - protein, 111 aa, synthetic construct
  REGION 1..111: note = anti-VB 2 Humanized VL 3
DIQLTQSPSSLSASVGDRVTITCRASKSVSILGTHLIHWYQQKPGKAPKLLIYAASNLESGVPSRFSGSGSETVFTLTISSLQPEDFATYFCQQSIEDPWTFGGGTKLEIK

SEQ ID NO: 299 - protein, 111 aa, synthetic construct
  REGION 1..111: note = anti-VB 2 Humanized VL 4
DIQLTQSPSSLSASVGDRVTITCRASKSVSILGTHLIHWYQQKPGKAPKLLIYAASNLESGVPSRFSGSGSETDFTLTISSLQPEDFATYYCQQSIEDPWTFGGGTKLEIK

SEQ ID NO: 300 - protein, 111 aa, synthetic construct
  REGION 1..111: note = anti-VB 2 Humanized VL 5
DIVLTQSPASLAVSLGERATISCRASKSVSILGTHLIHWYQQKPGQPPKLLIYAASNLESGVPDRFSGSGSGTDFTLTISRVEAEDVAVYYCQQSIEDPWTFGGGTKLEIK

SEQ ID NO: 301 - protein, 111 aa, synthetic construct
  REGION 1..111: note = anti-VB 2 Humanized VL 6
DIVLTQSPASLAVSPGERATISCRASKSVSILGTHLIHWYQQKPGQPPKLLIYAASNLESGVPDRFSGSGSGTDFTLTISRVEAEDVAVYYCQQSIEDPWTFGQGTKVEIK

SEQ ID NO: 302 - protein, 111 aa, synthetic construct
  REGION 1..111: note = anti-VB 2 Humanized VL 7
DIVMTQSPDSLAVSLGERATINCRASKSVSILGTHLIHWYQQKPGQPPKLLIYAASNLESGVPDRFSGSGSGTDFTLTISSLQAEDVAVYYCQQSIEDPWTFGQGTKVEIK

SEQ ID NO: 303 - protein, 111 aa, synthetic construct
  REGION 1..111: note = anti-VB 2 Humanized VL 8
EIVLTQSPATLSLSPGERATLSCRASKSVSILGTHLIHWYQQKPGQSPRLLIYAASNLESGVPDRFSGSGSGTDFTLTISRLEAEDVAVYYCQQSIEDPWTFGQGTKVEIK

SEQ ID NO: 304 - protein, 107 aa, synthetic construct
  REGION 1..107: note = anti-VB 7.2 Humanized VL 1
DIQMTQSPSFLSASVGDRVTITCKASQDVYTAVAWYQQKPGQAPKLLIYSASNRYTGVPSRFSGSGSGTDFTLTISSLQAEDLATYYCQQHYTTPRTFGQGTKVEIK

SEQ ID NO: 305 - protein, 107 aa, synthetic construct
  REGION 1..107: note = anti-VB 7.2 Humanized VL 2
DIQMTQSPSSLSASVGDRVTITCKASQDVYTAVAWYQQKPGKAPKLLIYSASNRYTGVPSRFSGSGSGTDFTFTISSLQPEDIATYYCQQHYTTPRTFGQGTKLEIK

SEQ ID NO: 306 - protein, 107 aa, synthetic construct
  REGION 1..107: note = anti-VB 7.2 Humanized VL 3
DIQMTQSPSSVSASVGDRVTITCKASQDVYTAVAWYQQKPGKAPKLLIYSASNRYTGVPSRFSGSGSGTDFTLTISSLQPEDFATYYCQQHYTTPRTFGQGTKLEIK

SEQ ID NO: 307 - protein, 107 aa, synthetic construct
  REGION 1..107: note = anti-VB 7.2 Humanized VL 4
AIQLTQSPSSLSASVGDRVTITCKASQDVYTAVAWYQQKPGKAPKLLIYSASNRYTGVPSRFSGSGSGTDFTLTISSLQPEDFATYYCQQHYTTPRTFGQGTKLEIK

SEQ ID NO: 308 - protein, 107 aa, synthetic construct
  REGION 1..107: note = anti-VB 4 Humanized VL 1
DIQMTQSPSSVSASVGDRVTITCLASEGISNFLAWYQQKPGKAPKLLIYYTSSLQDGVPSRFSGSGSGTDFTLTISSLQPEDFATYYCQQGYKFPRTFGQGTKLEIK

SEQ ID NO: 309 - protein, 107 aa, synthetic construct
  REGION 1..107: note = anti-VB 4 Humanized VL 2
DIQMTQSPSSLSASVGDRVTITCLASEGISNFLAWFQQKPGKAPKSLIYYTSSLQDGVPSRFSGSGSGTDFTLTISSLQPEDFATYYCQQGYKFPRTFGQGTKLEIK

SEQ ID NO: 310 - protein, 108 aa, synthetic construct
  REGION 1..108: note = anti-VB 13.2 Humanized VL 1
EIVLTQSPATLSVSPGEKVTITCSASSSISSTNLHWYQQKPGQSPRLLIYGTSNLASGVPDRFSGSGSGTDFTLTISSLEAEDFATYYCQQWSRYPLTFGQGTKLEIK

SEQ ID NO: 311 - protein, 108 aa, synthetic construct
  REGION 1..108: note = anti-VB 13.2 Humanized VL 2
EIVLTQSPATLSLSPGERATLSCSASSSISSTNLHWYQQKPGQAPRLLIYGTSNLASGIPDRFSGSGSGTDFTLTISSLEPEDFAVYYCQQWSRYPLTFGQGTKLEIK

SEQ ID NO: 312 - protein, 108 aa, synthetic construct
  REGION 1..108: note = anti-VB 13.2 Humanized VL 3
EIVLTQSPATLSLSPGERATLSCSASSSISSTNLHWYQQKPGQAPRLLIYGTSNLASGIPDRFSGSGSGTDFTLTISRLEPEDFAVYYCQQWSRYPLTFGQGTKLEIK

SEQ ID NO: 313 - protein, 108 aa, synthetic construct
  REGION 1..108: note = anti-VB 13.2 Humanized VL 4
EIVLTQSPGTLSLSPGERATLSCSASSSISSTNLHWYQQKPGQAPRLLIYGTSNLASGIPDRFSGSGSGTDFTLTISRLEPEDFAVYYCQQWSRYPLTFGQGTKLEIK

SEQ ID NO: 314 - protein, 108 aa, synthetic construct
  REGION 1..108: note = anti-VB 13.2 Humanized VL 5
EIQLTQSPSSLSASVGDRVTITCRASSSISSTNLHWYQQKPGKAPKPWIYGTSNLASGVPSRFSGSGSGTSYSLTISSLQPEDFATYYCQQWSRYPLTFGGGTKLEIK

SEQ ID NO: 315 - protein, 108 aa, synthetic construct
  REGION 1..108: note = anti-VB 13.2 Humanized VL 6
AIQLTQSPSSLSASVGDRVTITCRASSSISSTNLHWYQQKPGKAPKPLIYGTSNLASGVPSRFSGSGSGTDYTLTISSLQPEDFATYYCQQWSRYPLTFGGGTKLEIK

SEQ ID NO: 316 - protein, 108 aa, synthetic construct
  REGION 1..108: note = anti-VB 13.2 Humanized VL 7
EIVLTQSPATLSLSPGERATLSCRASSSISSTNLHWYQQKPGQAPRPWIYGTSNLASGVPARFSGSGSGTSYSLTISSLEPEDFATYYCQQWSRYPLTFGGGTKLEIK

SEQ ID NO: 317 - protein, 108 aa, synthetic construct
  REGION 1..108: note = anti-VB 13.2 Humanized VL 8
EIVLTQSPATLSLSPGERATLSCRASSSISSTNLHWYQQKPGQAPRPLIYGTSNRATGIPARFSGSGSGTDYTLTISSLEPEDFAVYYCQQWSRYPLTFGGGTKLEIK

SEQ ID NO: 318 - protein, 111 aa, synthetic construct
  REGION 1..111: note = anti-VB 14 Humanized VL 1
DIVLTQSPASLAVSPGERATISCRASESVDSYGNSFMHWYQQKPGQPPKLLIYRASNLESGVPDRFSGSGSGTDFTLTISRVEADDVAVYYCQQSNEDPYTFGQGTKLEIK

SEQ ID NO: 319 - protein, 111 aa, synthetic construct
  REGION 1..111: note = anti-VB 14 Humanized VL 2
DIVLTQSPASLAVSPGERATISCRASESVDSYGNSFMHWYQQKPGQPPKLLIYRASNLESGVPDRFSGSGSGTDFTLTISRVEAEDVAVYYCQQSNEDPYTFGQGTKLEIK

SEQ ID NO: 320 - protein, 111 aa, synthetic construct
  REGION 1..111: note = anti-VB 14 Humanized VL 3
DIVMTQSPDSLAVSLGERATINCRASESVDSYGNSFMHWYQQKPGQPPKLLIYRASNLESGVPDRFSGSGSGTDFTLTISSLQAEDVAVYYCQQSNEDPYTFGQGTKLEIK

SEQ ID NO: 321 - protein, 111 aa, synthetic construct
  REGION 1..111: note = anti-VB 14 Humanized VL 4
EIVLTQSPATLSLSPGERATLSCRASESVDSYGNSFMHWYQQKPGQPPRLLIYRASNLESGVPDRFSGSGSGTDFTLTISRLEAEDVAVYYCQQSNEDPYTFGQGTKLEIK

SEQ ID NO: 322 - protein, 111 aa, synthetic construct
  REGION 1..111: note = anti-VB 14 Humanized VL 5
EIVLTQSPATLSLSPGERATLSCRASESVDSYGNSFMHWYQQKPGQPPRLLIYRASNLESGVPDRFSGSGSGTDFTLTISSLEAEDFAVYYCQQSNEDPYTFGQGTKLEIK

SEQ ID NO: 323 - protein, 111 aa, synthetic construct
  REGION 1..111: note = anti-VB 14 Humanized VL 6
EIVLTQSPGTLSLSPGERATLSCRASESVDSYGNSFMHWYQQKPGQPPRLLIYRASNLESGVPDRFSGSGSGTDFTLTISRLEAEDVAVYYCQQSNEDPYTFGQGTKLEIK

SEQ ID NO: 324 - protein, 109 aa, synthetic construct
  REGION 1..109: note = anti-VB 22 Humanized VL 1
QAVVTQEPSLTVSPGGTVTLTCRSSTGAVTTSNYANWFQQKPGHAPRTLIYGTNNRAPGVPARFSGSLLGDKAALTITGAQTEDEADYFCALWYSNHWVFGGGTKLTVL

SEQ ID NO: 325 - protein, 109 aa, synthetic construct
  REGION 1..109: note = anti-VB 22 Humanized VL 2
QAVVTQEPSLTVSPGGTVTLTCRSSTGAVTTSNYANWFQQKPGQAPRTLIYGTNNRAPWTPARFSGSLLGGKAALTLSGAQPEDEAEYYCALWYSNHWVFGGGTKLTVL

SEQ ID NO: 326 - protein, 109 aa, synthetic construct
  REGION 1..109: note = anti-VB 22 Humanized VL 3
QTVVTQEPSLTVSPGGTVTLTCRSSTGAVTTSNYANWFQQKPGQAPRALIYGTNNRAPWTPARFSGSLLGGKAALTLSGVQPEDEAEYYCALWYSNHWVFGGGTKLTVL

SEQ ID NO: 327 - protein, 109 aa, synthetic construct
  REGION 1..109: note = anti-VB 22 Humanized VL 4
QTVVTQEPSFSVSPGGTVTLTCRSSTGAVTTSNYANWYQQTPGQAPRTLIYGTNNRAPGVPDRFSGSILGNKAALTITGAQADDESDYYCALWYSNHWVFGGGTKLTVL

SEQ ID NO: 328 - protein, 107 aa, synthetic construct
  REGION 1..107: note = anti-VB 11 Humanized VL 1
DIQMTQSPSSLSASVGDRVTITCKASQDINSYLSWFQQKPGKAPKLLIYRANRLVDGVPSRFSGSGSGTDYTLTISSLQPEDFATYYCLQYDEFPFTFGQGTRLEIK

SEQ ID NO: 329 - protein, 107 aa, synthetic construct
  REGION 1..107: note = anti-VB 11 Humanized VL 2
DIQMTQSPSSLSASVGDRVTITCKASQDINSYLSWFQQKPGKAPKSLIYRANRLVDGVPSRFSGSGSGTDFTLTISSLQPEDFATYYCLQYDEFPFTFGQGTRLEIK

SEQ ID NO: 330 - protein, 107 aa, synthetic construct
  REGION 1..107: note = anti-VB 11 Humanized VL 3
DIQMTQSPSSLSASVGDRVTITCKASQDINSYLSWYQQKPGKAPKLLIYRANRLVDGVPSRFSGSGSGTDFTFTISSLQPEDIATYYCLQYDEFPFTFGQGTRLEIK

SEQ ID NO: 331 - protein, 107 aa, synthetic construct
  REGION 1..107: note = anti-VB 11 Humanized VL 4
DIQMTQSPSSLSASVGDRVTITCKASQDINSYLSWYQQKPGKAPKLLLYRANRLVDGVPSRFSGSGSGTDYTLTISSLQPEDFATYYCLQYDEFPFTFGQGTRLEIK

SEQ ID NO: 332 - protein, 107 aa, synthetic construct
  REGION 1..107: note = anti-VB 1 Humanized VL 1
DVQMTQSPSTLSASVGESVTITCKASKSINKYLAWYQQKPGKAPKLLIYDGSTLQSGVPSRFSGSGSGTDFTLTISGLQPEDFATYYCQQHNEYPPTFGQGTKLEIK

SEQ ID NO: 333 - protein, 107 aa, synthetic construct
  REGION 1..107: note = anti-VB 1 Humanized VL 2
DIQMTQSPSSLSASVGDRVTITCKASKSINKYLAWYQQKPGKAPKLLIYDGSTLQSGVPSRFSGSGSGTDFTLTISSLQPEDFATYYCQQHNEYPPTFGQGTKLEIK

SEQ ID NO: 334 - protein, 107 aa, synthetic construct
  REGION 1..107: note = anti-VB 1 Humanized VL 3
DIQMTQSPSTLSASVGDRVTITCKASKSINKYLAWYQQKPGKAPKLLIYDGSTLQSGVPSRFSGSGSGTEFTLTISSLQPDDFATYYCQQHNEYPPTFGQGTKLEIK

SEQ ID NO: 335 - protein, 107 aa, synthetic construct
  REGION 1..107: note = anti-VB 1 Humanized VL 4
DIQLTQSPSFLSASVGDRVTITCKASKSINKYLAWYQQKPGKAPKLLIYDGSTLQSGVPSRFSGSGSGTEFTLTISSLQPEDFATYYCQQHNEYPPTFGQGTKLEIK

SEQ ID NO: 336 - protein, 107 aa, synthetic construct
  REGION 1..107: note = anti-VB 1 Humanized VL 5
DIQMTQSPSSLSASVGDRVTITCKASKSINKYLAWFQQKPGKAPKSLIYDGSTLQSGVPSRFSGSGSGTDFTLTISSLQPEDFATYYCQQHNEYPPTFGQGTKLEIK

SEQ ID NO: 337 - protein, 108 aa, synthetic construct
  REGION 1..108: note = anti-VB 12 Humanized VL 1
EIVLTQSPSFLSASPGEKVTITCSVSSSVSYMEIWYQQKPGQAPKLLIYDTSKLASGVPARFSGSGSGTDFTLTISSLEAEDFATYYCQQWSSNPQYTFGQGTKLEIK

SEQ ID NO: 338 - protein, 108 aa, synthetic construct
  REGION 1..108: note = anti-VB 12 Humanized VL 2
AIQLTQSPSSLSASVGDRVTITCSVSSSVSYMEIWYQQKPGKAPKLLIYDTSKLASGVPSRFSGSGSGTDFTLTISSLQPEDFATYYCQQWSSNPQYTFGQGTKLEIK

SEQ ID NO: 339 - protein, 108 aa, synthetic construct
  REGION 1..108: note = anti-VB 12 Humanized VL 3
EIVLTQSPATLSLSPGERATLSCSVSSSVSYMEIWYQQKPGQAPRLLIYDTSKLASGIPARFSGSGSGTDFTLTISSLEPEDFAVYYCQQWSSNPQYTFGQGTKLEIK

SEQ ID NO: 340 - protein, 108 aa, synthetic construct
  REGION 1..108: note = anti-VB 12 Humanized VL 4
DIQMTQSPSSLSASVGDRVTITCSVSSSVSYMEIWYQQKPGKAPKLLIYDTSKLASGVPSRFSGSGSGTDFTLTISSLQPEDFATYYCQQWSSNPQYTFGQGTKLEIK

SEQ ID NO: 341 - protein, 108 aa, synthetic construct
  REGION 1..108: note = anti-VB 12 Humanized VL 5
DIQLTQSPSFLSASVGDRVTITCSVSSSVSYMEIWYQQKPGKAPKLLIYDTSKLASGVPSRFSGSGSGTEFTLTISSLQPEDFATYYCQQWSSNPQYTFGQGTKLEIK

SEQ ID NO: 342 - protein, 106 aa, synthetic construct
  REGION 1..106: note = anti-VB 8 Humanized VL 1
EIVLTQSPAILSVSLGEKVTLSCRASSSVNYIYWYQQKPGKAPKLLIYYTSNLAPGVPTRFSGSGSGTDFSLTISSLQPEDAATYYCQQFTSSPFTFGQGTKLEIK

SEQ ID NO: 343 - protein, 106 aa, synthetic construct
  REGION 1..106: note = anti-VB 8 Humanized VL 2
EIVLTQSPATLSLSPGERATLSCRASSSVNYIYWYQQKPGQAPRLLIYYTSNLAPGIPARFSGSGSGTDFTLTISSLEPEDFAVYYCQQFTSSPFTFGQGTKLEIK

SEQ ID NO: 344 - protein, 106 aa, synthetic construct
  REGION 1..106: note = anti-VB 8 Humanized VL 3
DIQLTQSPSSLSASVGDRVTITCRASSSVNYIYWYQQKPGKAPKLLIYYTSNLAPGVPSRFSGSGSGTDFTLTISSLQPEDFATYYCQQFTSSPFTFGQGTKLEIK

SEQ ID NO: 345 - protein, 106 aa, synthetic construct
  REGION 1..106: note = anti-VB 13.1 Humanized VL 1
EIVLTQSPAILSASPGEKVTLTCSAGSSVSFMEIWYQQKPGQSPKLLIYDTSKLASGVPGRFSGSGSGTDFSLTISRLEPDDAAYYYLQGSGFPLTFGQGTKLEIK

SEQ ID NO: 346 - protein, 107 aa, synthetic construct
  REGION 1..107: note = anti-VB 13.1 Humanized VL 2
EIVLTQSPATLSLSPGERATLSCSAGSSVSFMEIWYQQKPGQAPRLLIYDTSKLASGIPARFSGSGSGTDFTLTISSLEPEDFAVYYCLQGSGFPLTFGQGTKLEIK

SEQ ID NO: 347 - protein, 107 aa, synthetic construct
  REGION 1..107: note = anti-VB 13.1 Humanized VL 3
EIVLTQSPATLSLSPGERATLSCSAGSSVSFMEIWYQQKPGQAPRLLIYDTSKLASGIPDRFSGSGSGTDFTLTISRLEPEDFAVYYCLQGSGFPLTFGQGTKLEIK

SEQ ID NO: 348 - protein, 106 aa, synthetic construct
  REGION 1..106: note = anti-VB 5.1 Humanized VL 1
EIVLTQSPAILAVSLGEKVTITCSASSSVSYMHWYQQKPGKAPKLLIYEISKLASGVPARFSGSGSGTSFSLTISSLQAEDAATYYCQQWNYPLLTFGQGTKLEIK

SEQ ID NO: 349 - protein, 106 aa, synthetic construct
  REGION 1..106: note = anti-VB 5.1 Humanized VL 2
DIQLTQSPSFLSASVGDRVTITCSASSSVSYMHWYQQKPGKAPKLLIYEISKLASGVPSRFSGSGSGTEFTLTISSLQPEDFATYYCQQWNYPLLTFGQGTKLEIK

SEQ ID NO: 350 - protein, 106 aa, synthetic construct
  REGION 1..106: note = anti-VB 5.1 Humanized VL 3
DIQLTQSPSSLSASVGDRVTITCSASSSVSYMHWYQQKPGKAPKLLIYEISKLASGVPSRFSGSGSGTDFTLTISSLQPEDFATYYCQQWNYPLLTFGQGTKLEIK

SEQ ID NO: 351 - protein, 107 aa, synthetic construct
  REGION 1..107: note = anti-VB 9 Humanized VL 1
ETVMTQSPASLSVAPGEKVTITCISSTDIDDDMNWYQQKPGEPPKLLISEGNTLRPGVPSRFSGSGYGTDFTFTINNLQSEDVADYYCLQSDNMPLTFGQGTKLEIK

SEQ ID NO: 352 - protein, 107 aa, synthetic construct
  REGION 1..107: note = anti-VB 9 Humanized VL 2
ETTLTQSPAFMSATPGDKVNISCISSTDIDDDMNWYQQKPGEAPIFIIQEGNTLRPGIPPRFSGSGYGTDFTLTINNIESEDAAYYFCLQSDNMPLTFGQGTKLEIK

SEQ ID NO: 353 - protein, 107 aa, synthetic construct
  REGION 1..107: note = anti-VB 9 Humanized VL 3
DIQMTQSPSSLSASVGDRVTITCISSTDIDDDMNWYQQKPGKAPKLLIYEGNTLRPGVPSRFSGSGSGTDFTFTISSLQPEDIATYYCLQSDNMPLTFGQGTKLEIK

SEQ ID NO: 354 - protein, 107 aa, synthetic construct
  REGION 1..107: note = anti-VB 9 Humanized VL 4
DVVMTQSPAFLSVTPGEKVTITCISSTDIDDDMNWYQQKPGQAPKLLIKEGNTLRPGVPSRFSGSGSGTDFTLTISSLEAEDAATYYCLQSDNMPLTFGQGTKLEIK

SEQ ID NO: 355 - protein, 112 aa, synthetic construct
  REGION 1..112: note = anti-VB 17 Humanized VL 1
DVVMTQTPLSLPVTLGQPASISCRSSQSLVHSNGNTYLHWYLQKPGQSPQLLIYKVSNRFSGVPDRFSGSGSGTDFTLKISRVEAEDVGVYYCSQSTHVPFTFGQGTKLEIK

SEQ ID NO: 356 - protein, 112 aa, synthetic construct
  REGION 1..112: note = anti-VB 17 Humanized VL 2
DVVMTQSPLSLPVTLGQPASISCRSSQSLVHSNGNTYLHWFQQRPGQSPRRLIYKVSNRFSGVPDRFSGSGSGTDFTLKISRVEAEDVGVYYCSQSTHVPFTFGQGTKLEIK

SEQ ID NO: 357 - protein, 112 aa, synthetic construct
  REGION 1..112: note = anti-VB 17 Humanized VL 3
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQSTHVPFTFGQGTKLEIK

SEQ ID NO: 358 - protein, 106 aa, synthetic construct
  REGION 1..106: note = anti-VB 3 Humanized VL 1
EIVLTQSPAILSASLGERITLTCSANSTVGYIEIWYQQKPGQAPKLLIYTTSNLASGVPSRFSGSGSGTDFSLTISSLEAEDFAVYFCHQWSFYPTFGQGTKLEIK

SEQ ID NO: 359 - protein, 106 aa, synthetic construct
  REGION 1..106: note = anti-VB 3 Humanized VL 2
EIVLTQSPATLSLSPGERATLSCSANSTVGYIEIWYQQKPGQAPRLLIYTTSNLASGIPARFSGSGSGTDFTLTISSLEPEDFAVYYCHQWSFYPTFGQGTKLEIK

SEQ ID NO: 360 - protein, 106 aa, synthetic construct
  REGION 1..106: note = anti-VB 3 Humanized VL 3
EIVLTQSPATLSLSPGERATLSCSANSTVGYIEIWYQQKPGQAPRLLIYTTSNLASGIPARFSGSGPGTDFTLTISSLEPEDFAVYYCHQWSFYPTFGQGTKLEIK

SEQ ID NO: 361 - protein, 111 aa, synthetic construct
  REGION 1..111: note = anti-VB 7.1 Humanized VL 1
DIVLTQSPVSLPVSPGERATISCRASKSVSTSGYSYMHWYQQKPGQPPKLLIYLASNLESGVPDRFSGSGSGTDFTLTISRVEAEDVAVYYCQHSRDLPWTFGGGTKLEIK

SEQ ID NO: 362 - protein, 111 aa, synthetic construct
  REGION 1..111: note = anti-VB 7.1 Humanized VL 2
DIVLTQSPASLAVSPGQRATITCRASKSVSTSGYSYMHWYQQKPGQPPKLLIYLASNLESGVPARFSGSGSGTDFTLTINPVEANDTANYYCQHSRDLPWTFGQGTKVEIK

SEQ ID NO: 363 - protein, 111 aa, synthetic construct
  REGION 1..111: note = anti-VB 7.1 Humanized VL 3
DIVMTQSPLSLPVTPGEPASISCRASKSVSTSGYSYMHWYLQKPGQSPQLLIYLASNLESGVPDRFSGSGSGTDFTLKISRVEAEDVGVYYCQHSRDLPWTFGQGTKVEIK

SEQ ID NO: 364 - protein, 111 aa, synthetic construct
  REGION 1..111: note = anti-VB 7.1 Humanized VL 4
EIVLTQSPGTLSLSPGERATLSCRASKSVSTSGYSYMHWYQQKPGQPPRLLIYLASNLESGVPDRFSGSGSGTDFTLTISRLEAEDVAVYYCQHSRDLPWTFGQGTKVEIK

SEQ ID NO: 365 - protein, 111 aa, synthetic construct
  REGION 1..111: note = anti-VB 7.1 Humanized VL 5
EIVLTQSPATLSLSPGERATLSCRASKSVSTSGYSYMHWYQQKPGQPPRLLIYLASNLESGVPDRFSGSGSGTDFTLTISRLEAEDVAVYYCQHSRDLPWTFGQGTKVEIK

SEQ ID NO: 366 - protein, 111 aa, synthetic construct
  REGION 1..111: note = anti-VB 7.1 Humanized VL 6
DIVMTQSPDSLAVSLGERATINCRASKSVSTSGYSYMHWYQQKPGQPPKLLIYLASNLESGVPDRFSGSGSGTDFTLTISSLQAEDVAVYYCQHSRDLPWTFGQGTKVEIK

SEQ ID NO: 367 - protein, 111 aa, synthetic construct
  REGION 1..111: note = anti-VB 5.3 Humanized VL 1
DIVLTQSPASLAVSPGERATISCRASESVDSSGNSFMHWYQQKPGQPPQLLIYRASNLESGVPDRFSGSGSGTDFTLTISRVEADDVAVYYCQQSFDDPFTFGQGTKLEIK

SEQ ID NO: 368 - protein, 111 aa, synthetic construct
  REGION 1..111: note = anti-VB 5.3 Humanized VL 2
DIVLTQSPASLAVSPGQRATITCRASESVDSSGNSFMHWYQQKPGQPPKLLIYRASNLESGVPARFSGSGSGTDFTLTINPVEANDTANYYCQQSFDDPFTFGQGTKLEIK

SEQ ID NO: 369 - protein, 111 aa, synthetic construct
  REGION 1..111: note = anti-VB 5.3 Humanized VL 3
EIVLTQSPATLSLSPGERATLSCRASESVDSSGNSFMHWYQQKPGQAPRLLIYRASNLESGIPARFSGSGSGTDFTLTISSLEPEDFAVYYCQQSFDDPFTFGQGTKLEIK

SEQ ID NO: 370 - protein, 5 aa, synthetic construct
  REGION 1..5: note = VB7.2 VH CDR1
DYTMH

SEQ ID NO: 371 - protein, 17 aa, synthetic construct
  REGION 1..17: note = VB7.2 VH CDR2
HINPSSGYSTYNQKFKD

SEQ ID NO: 372 - protein, 8 aa, synthetic construct
  REGION 1..8: note = VB7.2 VH CDR3
SLQLGRDY

SEQ ID NO: 373 - protein, 5 aa, synthetic construct
  REGION 1..5: note = VB4 VH CDR1
SYYIY

SEQ ID NO: 374 - protein, 17 aa, synthetic construct
  REGION 1..17: note = VB4 VH CDR2
YIYPGNGGTYYSEKFKG

SEQ ID NO: 375 - protein, 11 aa, synthetic construct
  REGION 1..11: note = VB4 VH CDR3
GSGDRYNSLAY

SEQ ID NO: 376 - protein, 5 aa, synthetic construct
  REGION 1..5: note = VB13.2 VH CDR1
DYYIH

SEQ ID NO: 377 - protein, 17 aa, synthetic construct
  REGION 1..17: note = VB13.2 VH CDR2
IVNTNNGDTNYNQRFKG

SEQ ID NO: 378 - protein, 11 aa, synthetic construct
  REGION 1..11: note = VB13.2 VH CDR3
ALYTGSYWFAY

SEQ ID NO: 379 - protein, 7 aa, synthetic construct
  REGION 1..7: note = VB14 VH CDR1
GFKTEDT

SEQ ID NO: 380 - protein, 6 aa, synthetic construct
  REGION 1..6: note = VB14 VH CDR2
DPANGN

SEQ ID NO: 381 - protein, 9 aa, synthetic construct
  REGION 1..9: note = VB14 VH CDR3
GSYYYAMDY

SEQ ID NO: 382 - protein, 5 aa, synthetic construct
  REGION 1..5: note = VB22 VH CDR1
RYWMN

SEQ ID NO: 383 - protein, 17 aa, synthetic construct
  REGION 1..17: note = VB22 VH CDR2
EINSDSNTINYTPSLKD

SEQ ID NO: 384 - protein, 7 aa, synthetic construct
  REGION 1..7: note = VB22 VH CDR3
GGLLRDV

SEQ ID NO: 385 - protein, 5 aa, synthetic construct
  REGION 1..5: note = VB11 VH CDR1
DSAMH

SEQ ID NO: 386 - protein, 17 aa, synthetic construct
  REGION 1..17: note = VB11 VH CDR2
VISSYNGNTNYNQKFKG

SEQ ID NO: 387 - protein, 7 aa, synthetic construct
  REGION 1..7: note = VB11 VH CDR3
SRDAMDY

SEQ ID NO: 388 - protein, 7 aa, synthetic construct
  REGION 1..7: note = VB1 VH CDR1
GYTFTDY

SEQ ID NO: 389 - protein, 6 aa, synthetic construct
  REGION 1..6: note = VB1 VH CDR2
NTYTGT

SEQ ID NO: 390 - protein, 13 aa, synthetic construct
  REGION 1..13: note = VB1 VH CDR3
SWRRGIRGIGFDY

SEQ ID NO: 391 - protein, 7 aa, synthetic construct
  REGION 1..7: note = VB12 VH CDR1
GFTFRSY

SEQ ID NO: 392 - protein, 6 aa, synthetic construct
  REGION 1..6: note = VB12 VH CDR2
SSGGSY

SEQ ID NO: 393 - protein, 8 aa, synthetic construct
  REGION 1..8: note = VB12 VH CDR3
HGGNFFDY

SEQ ID NO: 394 - protein, 7 aa, synthetic construct
  REGION 1..7: note = VB8 VH CDR1
GFTFSNF

SEQ ID NO: 395 - protein, 6 aa, synthetic construct
  REGION 1..6: note = VB8 VH CDR2
SSGSST

SEQ ID NO: 396 - protein, 8 aa, synthetic construct
  REGION 1..8: note = VB8 VH CDR3
RGEGAMDY

SEQ ID NO: 397 - protein, 7 aa, synthetic construct
  REGION 1..7: note = VB13.1 VH CDR1
GFTFRSY

SEQ ID NO: 398 - protein, 6 aa, synthetic construct
  REGION 1..6: note = VB13.1 VH CDR2
SNGGDY

SEQ ID NO: 399 - protein, 12 aa, synthetic construct
  REGION 1..12: note = VB13.1 VH CDR3
PSYYSDPWFFDV

SEQ ID NO: 400 - protein, 10 aa, synthetic construct
  REGION 1..10: note = VB5.1 VH CDR1
GYTFTDYNIH

SEQ ID NO: 401 - protein, 6 aa, synthetic construct
  REGION 1..6: note = VB5.1 VH CDR2
NPYNGR

SEQ ID NO: 402 - protein, 9 aa, synthetic construct
  REGION 1..9: note = VB5.1 VH CDR3
WDGSSYFDY

SEQ ID NO: 403 - protein, 5 aa, synthetic construct
  REGION 1..5: note = VB9 VH CDR1
NTFMH

SEQ ID NO: 404 - protein, 17 aa, synthetic construct
  REGION 1..17: note = VB9 VH CDR2
RIDPTNGYTKFAPKFQG

SEQ ID NO: 405 - protein, 9 aa, synthetic construct
  REGION 1..9: note = VB9 VH CDR3
DYDAPWFAY

SEQ ID NO: 406 - protein, 8 aa, synthetic construct
  REGION 1..8: note = VB17 VH CDR1
GYSITSGY

SEQ ID NO: 407 - protein, 6 aa, synthetic construct
  REGION 1..6: note = VB17 VH CDR2
SYDGSN

SEQ ID NO: 408 - protein, 11 aa, synthetic construct
  REGION 1..11: note = VB17 VH CDR3
PSPGTGYAVDY

SEQ ID NO: 409 - protein, 7 aa, synthetic construct
  REGION 1..7: note = VB3 VH CDR1
GYTFSGS

SEQ ID NO: 410 - protein, 6 aa, synthetic construct
  REGION 1..6: note = VB3 VH CDR2
YPGDGD

SEQ ID NO: 411 - protein, 12 aa, synthetic construct
  REGION 1..12: note = VB3 VH CDR3
SGYFNYVPVFDY

SEQ ID NO: 412 - protein, 7 aa, synthetic construct
  REGION 1..7: note = VB7.1 VH CDR1
GFTFSDY

SEQ ID NO: 413 - protein, 6 aa, synthetic construct
  REGION 1..6: note = VB7.1 VH CDR2
SGGGSY

SEQ ID NO: 414 - protein, 14 aa, synthetic construct
  REGION 1..14: note = VB7.1 VH CDR3
ERDIYYGNFNAMVY

SEQ ID NO: 415 - protein, 7 aa, synthetic construct
  REGION 1..7: note = VB5.3 VH CDR1
GYAFSSS

SEQ ID NO: 416 - protein, 6 aa, synthetic construct
  REGION 1..6: note = VB5.3 VH CDR2
YPGDGD

SEQ ID NO: 417 - protein, 10 aa, synthetic construct
  REGION 1..10: note = VB5.3 VH CDR3
RGTGGWYFDV

SEQ ID NO: 418 - protein, 11 aa, synthetic construct
  REGION 1..11: note = VB7.2 VL CDR1
KASQDVYTAVA

SEQ ID NO: 419 - protein, 6 aa, synthetic construct
  REGION 1..6: note = VB7.2 VL CDR2
ASNRYT

SEQ ID NO: 420 - protein, 9 aa, synthetic construct
  REGION 1..9: note = VB7.2 VL CDR3
QQHYTTPRT

SEQ ID NO: 421 - protein, 11 aa, synthetic construct
  REGION 1..11: note = VB4 VL CDR1
LASEGISNFLA

SEQ ID NO: 422 - protein, 7 aa, synthetic construct
  REGION 1..7: note = VB4 VL CDR2
YTSSLQD

SEQ ID NO: 423 - protein, 9 aa, synthetic construct
  REGION 1..9: note = VB4 VL CDR3
QQGYKFPRT

SEQ ID NO: 424 - protein, 12 aa, synthetic construct
  REGION 1..12: note = VB13.2 VL CDR1
SASSSISSTNLH

SEQ ID NO: 425 - protein, 7 aa, synthetic construct
  REGION 1..7: note = VB13.2 VL CDR2
GTSNLAS

SEQ ID NO: 426 - protein, 9 aa, synthetic construct
  REGION 1..9: note = VB13.2 VL CDR3
QQWSRYPLT

SEQ ID NO: 427 - protein, 11 aa, synthetic construct
  REGION 1..11: note = VB14 VL CDR1
SESVDSYGNSF

SEQ ID NO: 428 - protein, 7 aa, synthetic construct
  REGION 1..7: note = VB14 VL CDR2
RASNLES

SEQ ID NO: 429 - protein, 9 aa, synthetic construct
  REGION 1..9: note = VB14 VL CDR3
QQSNEDPYT

SEQ ID NO: 430 - protein, 14 aa, synthetic construct
  REGION 1..14: note = VB22 VL CDR1
RSSTGAVTTSNYAN

SEQ ID NO: 431 - protein, 7 aa, synthetic construct
  REGION 1..7: note = VB22 VL CDR2
GTNNRAP

SEQ ID NO: 432 - protein, 9 aa, synthetic construct
  REGION 1..9: note = VB22 VL CDR3
ALWYSNHWV

SEQ ID NO: 433 - protein, 11 aa, synthetic construct
  REGION 1..11: note = VB11 VL CDR1
KASQDINSYLS

SEQ ID NO: 434 - protein, 7 aa, synthetic construct
  REGION 1..7: note = VB11 VL CDR2
RANRLVD

SEQ ID NO: 435 - protein, 9 aa, synthetic construct
  REGION 1..9: note = VB11 VL CDR3
LQYDEFPFT

SEQ ID NO: 436 - protein, 7 aa, synthetic construct
  REGION 1..7: note = VB1 VL CDR1
SKSINKY

SEQ ID NO: 437 - protein, 7 aa, synthetic construct
  REGION 1..7: note = VB1 VL CDR2
DGSTLQS

SEQ ID NO: 438 - protein, 9 aa, synthetic construct
  REGION 1..9: note = VB1 VL CDR3
QQHNEYPPT

SEQ ID NO: 439 - protein, 6 aa, synthetic construct
  REGION 1..6: note = VB12 VL CDR1
SSSVSY

SEQ ID NO: 440 - protein, 7 aa, synthetic construct
  REGION 1..7: note = VB12 VL CDR2
DTSKLAS

SEQ ID NO: 441 - protein, 10 aa, synthetic construct
  REGION 1..10: note = VB12 VL CDR3
QQWSSNPQYT

SEQ ID NO: 442 - protein, 10 aa, synthetic construct
  REGION 1..10: note = VB8 VL CDR1
RASSSVNYIY

SEQ ID NO: 443 - protein, 7 aa, synthetic construct
  REGION 1..7: note = VB8 VL CDR2
YTSNLAP

SEQ ID NO: 444 - protein, 9 aa, synthetic construct
  REGION 1..9: note = VB8 VL CDR3
QQFTSSPFT

SEQ ID NO: 445 - protein, 6 aa, synthetic construct
  REGION 1..6: note = VB13.1 VL CDR1
GSSVSF

SEQ ID NO: 446 - protein, 7 aa, synthetic construct
  REGION 1..7: note = VB13.1 VL CDR2
DTSKLAS

SEQ ID NO: 447 - protein, 9 aa, synthetic construct
  REGION 1..9: note = VB13.1 VL CDR3
LQGSGFPLT

SEQ ID NO: 448 - protein, 6 aa, synthetic construct
  REGION 1..6: note = VB5.1 VL CDR1
SSSVSY

SEQ ID NO: 449 - protein, 7 aa, synthetic construct
  REGION 1..7: note = VB5.1 VL CDR2
EISKLAS

SEQ ID NO: 450 - protein, 9 aa, synthetic construct
  REGION 1..9: note = VB5.1 VL CDR3
QQWNYPLLT

SEQ ID NO: 451 - protein, 11 aa, synthetic construct
  REGION 1..11: note = VB9 VL CDR1
ISSTDIDDDMN

SEQ ID NO: 452 - protein, 7 aa, synthetic construct
  REGION 1..7: note = VB9 VL CDR2
EGNTLRP

SEQ ID NO: 453 - protein, 9 aa, synthetic construct
  REGION 1..9: note = VB9 VL CDR3
LQSDNMPLT

SEQ ID NO: 454 - protein, 12 aa, synthetic construct
  REGION 1..12: note = VB17 VL CDR1
SQSLVHSNGNTY

SEQ ID NO: 455 - protein, 7 aa, synthetic construct
  REGION 1..7: note = VB17 VL CDR2
KVSNRFS

SEQ ID NO: 456 - protein, 9 aa, synthetic construct
  REGION 1..9: note = VB17 VL CDR3
SQSTHVPFT

SEQ ID NO: 457 - protein, 6 aa, synthetic construct
  REGION 1..6: note = VB3 VL CDR1
NSTVGY

SEQ ID NO: 458 - protein, 7 aa, synthetic construct
  REGION 1..7: note = VB3 VL CDR2
TTSNLAS

SEQ ID NO: 459 - protein, 8 aa, synthetic construct
  REGION 1..8: note = VB3 VL CDR3
HQWSFYPT

SEQ ID NO: 460 - protein, 11 aa, synthetic construct
  REGION 1..11: note = VB7.1 VL CDR1
SKSVSTSGYSY

SEQ ID NO: 461 - protein, 7 aa, synthetic construct
  REGION 1..7: note = VB7.1 VL CDR2
LASNLES

SEQ ID NO: 462 - protein, 9 aa, synthetic construct
  REGION 1..9: note = VB7.1 VL CDR3
QHSRDLPWT

SEQ ID NO: 463 - protein, 11 aa, synthetic construct
  REGION 1..11: note = VB5.3 VL CDR1
SESVDSSGNSF

SEQ ID NO: 464 - protein, 7 aa, synthetic construct
  REGION 1..7: note = VB5.3 VL CDR2
RASNLES

SEQ ID NO: 465 - protein, 9 aa, synthetic construct
  REGION 1..9: note = VB5.3 VL CDR3
QQSFDDPFT

SEQ ID NO: 466 - protein, 119 aa, synthetic construct
  REGION 1..119: note = VB 2 murine VH
EVQLQQSVADLVRPGASLKLSCTASGFNIKSAYMHWVIQRPDQGPECLGRIDPATGKTKYAPKFQAKATITADTSSNTAYLQLSSLTSEDTAIYYCTRSLNWDYGLDYWGQGTSVTVSS

SEQ ID NO: 467 - protein, 111 aa, synthetic construct
  REGION 1..111: note = VB 2 murine VL
DIVLTQSPASLAVSLGQRATISCRASKSVSILGTHLIHWYQQKPGQPPKLLIYAASNLESGVPARFSGSGSETVFTLNIHPVEEEDAATYFCQQSIEDPWTFGGGTKLGIK

SEQ ID NO: 468 - protein, 117 aa, synthetic construct
  REGION 1..117: note = VB 7.2 murine VH
QVHLQQSGAELARPGASVKMSCKASGYIFTDYTMHWVKQRPGQCLEWIGHINPSSGYSTYNQKFKDKATLTADKSSSTAYMQLSSLTSEDSAVYYCARSLQLGRDYWGQGTTLTVSS

SEQ ID NO: 469 - protein, 107 aa, synthetic construct
  REGION 1..107: note = VB 7.2 murine VL
DIVMTQSHKFMSTSVGDRVSITCKASQDVYTAVAWYQQKPGQSPKLLIYSASNRYTGVPDRFTGSGSGTDFTFTISSVQAEDLAVYYCQQHYTTPRTFGCGTKLEIK

SEQ ID NO: 470 - protein, 120 aa, synthetic construct
  REGION 1..120: note = VB 4 murine VH
QVQLQQSGAELAKPGTSVKLSCKASGYTFTSYYIYWVKQRPGQCLEWLGYIYPGNGGTYYSEKFKGKATFTADTSSNTAYMLLGSLTPEDSAYYFCARGSGDRYNSLAYWGQGTLVTVSS

SEQ ID NO: 471 - protein, 107 aa, synthetic construct; identical to SEQ ID NO: 164
  REGION 1..107: note = VB 4 murine VL

SEQ ID NO: 472 - protein, 120 aa, synthetic construct
  REGION 1..120: note = VB 13.2 murine VH
EVQLQQSGPELVKPGASVKMSCNASGYTFTDYYIHWLKQRHGKCLEWIGIVNTNNGDTNYNQRFKGKASLTVDKSSSTAYMELNSLTSEDSAVFYCARALYTGSYWFAYWGQGTLVTVSA

SEQ ID NO: 473 - protein, 108 aa, synthetic construct
  REGION 1..108: note = VB 13.2 murine VL
EIVLTQSPAVMAASPGEKVTITCSASSSISSTNLHWYQQKSETSPKPWIYGTSNLASGVPVRFSGSGSGTSYSLTISSIEAEDAATYYCQQWSRYPLTFGCGTKLEII

SEQ ID NO: 474 - protein, 118 aa, synthetic construct
  REGION 1..118: note = VB 14 murine VH
EVQLQQSGAELVKPGASVKLSCTASGFKTEDTYMYWVKQRPEQCLEWIGRIDPANGNTKYDPKFQDKATITADSSSNTAYLQLSSLPSEDTAVYYCARGSYYYAMDYWGQGTSVTVSS

SEQ ID NO: 475 - protein, 111 aa, synthetic construct
  REGION 1..111: note = VB 14 murine VL
DIVLTQSPASLAVSLGQRATISCRASESVDSYGNSFMHWYQQKPGQPPKLLIYRASNLESGIPARFSGSGSRTDFTLTINPVEADDVATYYCQQSNEDPYTFGCGTKLEIK

SEQ ID NO: 476 - protein, 116 aa, synthetic construct
  REGION 1..116: note = VB22 murine VH
EVKLLESGGGLVQPGGSLKLSCAASGFDFSRYWMNWVRQAPGKCLEWIGEINSDSNTINYTPSLKDKFIISRDNAKNTLYLQMNKVRSEDTALYYCARGGLLRDVWGAGTTVTVSS

SEQ ID NO: 477 - protein, 116 aa, synthetic construct
  REGION 1..116: note = VB11 murine VH
QVQLQQSGPEVVRPGVSVKISCKGSGYRFTDSAMHWVKQSHAKCLEWIGVISSYNGNTNYNQKFKGKATMTVDKSSSTAYMELARMTSEDSAIYYCARSRDAMDYWGQGTSVTVSS

SEQ ID NO: 478 - protein, 107 aa, synthetic construct
  REGION 1..107: note = VB11 murine VL
DIKMTQSPSSMYASLGERVTITCKASQDINSYLSWFQQKAGKSPKTLIYRANRLVDGVPSRFSGSGSGQDYSLTISSLEYEDMGIYYCLQYDEFPFTFGCGTRLEIK

SEQ ID NO: 479 - protein, 121 aa, synthetic construct
  REGION 1..121: note = VB1 murine VH
QLQLVQSGPELREPGESVKISCKASGYTFTDYIVHWVKQAPGKCLKWMGWINTYTGTPTYADDFEGRFVFSLEASASTANLQISNLKNEDTATYFCARSWRRGIRGIGFDWGQGVMVTVSS

SEQ ID NO: 480 - protein, 107 aa, synthetic construct
  REGION 1..107: note = VB1 murine VL
DVQMTQSPYNLAASPGESVSINCKASKSINKYLAWYQQKPGKPNKLLIYDGSTLQSGIPSRFSGSGSGTDFTLTIRGLEPEDFGLYYCQQHNEYPPTFGCGTKLELK

SEQ ID NO: 481 - protein, 117 aa, synthetic construct
  REGION 1..117: note = VB12 murine VH
EVQLVESGGDLVKPGGSLKLSCAVSGFTFRSYGMSWVRQTPDKCLEWVALISSGGSYTYYTDSVKGRFTISRDNAKNTLYLQMSSLKSEDTAIYYCSRHGGNFFDYWGQGTTLTVSS

SEQ ID NO: 482 - protein, 108 aa, synthetic construct
  REGION 1..108: note = VB12 murine VL
QIVLTQSPSIMSASPGEKVTMTCSVSSSVSYMEIWYQQKSGTSPKRWIYDTSKLASGVPARFSGSGSGTSYSLTISSMEAEDAATYYCQQWSSNPQYTFGCGTKLEIK

SEQ ID NO: 483 - protein, 117 aa, synthetic construct
  REGION 1..117: note = VB8 murine VH
EVQLVESGGGLVQPGGSRKLSCAASGFTFSNFGMHWVRQAPGKCLEWVAYISSGSSTIYYADTLKGRFTISRDNPKNTLFLQMTSLRSEDTAMYYCARRGEGAMDYWGQGTSVTVSS

SEQ ID NO: 484 - protein, 106 aa, synthetic construct
  REGION 1..106: note = VB8 murine VL
ENVLTQSPAIMSASLGEKVTMSCRASSSVNYIYQYQQKSDASPKLWIYYTSNLAPGVPTRFSGSGSGNSYSLTISSMEGEDAATYYCQQFTSSPFTFGCGTKLEIK

SEQ ID NO: 485 - protein, 121 aa, synthetic construct
  REGION 1..121: note = VB13.1 murine VH
DVKLVESGEGLVKPGGSLKLSCAASGFTFRSYAMSWVRQTPEKCLEWVAHISNGGDYIYYADTVKGRFTISRDNARNTLYLQMSSLKSEDTAMYYCTRPSYYSDPWFFDVWGTGTTVTVSS

SEQ ID NO: 486 - protein, 106 aa, synthetic construct
  REGION 1..106: note = VB13.1 murine VL
ENVLTQSPAIMSASPGEKVTMTCSAGSSVSFMEIWYQQKSSTSPKLWIYDTSKLASGVPGRFSGSGSGNSFSLTISMEAEDVAIYYCLQGSGFPLTFGCGTKLEIK

SEQ ID NO: 487 - protein, 118 aa, synthetic construct
  REGION 1..118: note = VB5.1 murine VH
EVQLQQSGPVLVKPGASVRMSCKASGYTFTDYNIHWVKQSHGRCLEWVGYINPYNGRTGYNQKFKAKATLTVDKSSSTAYMDLRSLTSEDSAVYYCARWDGSSYFDYWGQGTTLTVSS

SEQ ID NO: 488 - protein, 106 aa, synthetic construct
  REGION 1..106: note = VB5.1 murine VL
EIVLTQSPAITAASLGQKVTITCSASSSVSYMHWYQQKSGTSPKPWIYEISKLASGVPARFSGSGSGTSYSLTISSMEAEDAAIYYCQQWNYPLLTFGCGTKLELK

SEQ ID NO: 489 - protein, 118 aa, synthetic construct
  REGION 1..118: note = VB9 murine VH
EVQLQQSVAELVRPGASVKLSCTASGFNIKNTFMHWVKQRPEQCLEWIGRIDPTNGYTKFAPKFQGKATLTAVTSSNTVYLQLSSLTSEDTAIYYCAHDYDAPWFAYWGQGTLVIVSA

SEQ ID NO: 490 - protein, 107 aa, synthetic construct
  REGION 1..107: note = VB9 murine VL
ETTVTQSPASLSVATGEKVTIRCISSTDIDDDMNWYQQKSGEPPKLLISEGNTLRPGVPSRFSSSGYGTDFVFTIENMLSEDVADYYCLQSDNMPLTFGCGTKLELK

SEQ ID NO: 491 - protein, 120 aa, synthetic construct
  REGION 1..120: note = VB17 murine VH
NVQLQESGPGLVKPSQSLSLTCSVAGYSITSGYFWNWIRQFPGNCLEWMGYISYDGSNNYNPSLKNRISITRDTSKNQFFLKLNSVTTEDTATYYCASPSPGTGYAVDYWGQGTSVTVSS

SEQ ID NO: 492 - protein, 112 aa, synthetic construct
  REGION 1..112: note = VB17 murine VL
NVVMTQTPLSLPVSLGDQASISCRSSQSLVHSNGNTYLHWYLQKPGQSPKFLIYKVSNRFSGVPDRFSGGGSGTEFTLKISRVEAEDLGVYFCSQSTHVPFTFGCGTKLEIK

SEQ ID NO: 493 - protein, 121 aa, synthetic construct
  REGION 1..121: note = VB3 murine VH
QIQLQQSGPEVVKPGASVQISCKASGYTFSGSWMNWVKQRPGKCLEWIGRIYPGDGDTDYSGKFKGRATLTADKSSSTAYMRLSSLTSEDSAVYFCARSGYFNYVPVFDYWGQGTTLSVSS

SEQ ID NO: 494 - protein, 106 aa, synthetic construct
  REGION 1..106: note = VB3 murine VL
QIVLTQSPAIMSASLGEEIALTCSANSTVGYIEIWYQQKSGTSPKLLIYTTSNLASGVPSRFSGSGSGTFYSLTISSVEAEDAADYFCHQWSFYPTFGCGTKLEIK

SEQ ID NO: 495 - protein, 123 aa, synthetic construct
  REGION 1..123: note = VB7.1 murine VH
EVQLVESGGGLVKPGGSLKLSCAASGFTFSDYYMYWVRQTPEKCLEWVATISGGGSYTYSPDSVKGRFTISRDNAKNNLYLQMSSLRSEDTAMYFCARERDIYYGNFNAMVYWGRGTSVTVSS

SEQ ID NO: 496 - protein, 111 aa, synthetic construct
  REGION 1..111: note = VB7.1 murine VL
DIVLTQSPVSLTVSLGQRATISCRASKSVSTSGYSYMHWYQQKPGQPPKLLIYLASNLESGVPARFSGSGSGTDFTLNIHPVEEEDAATYYCQHSRDLPWTFGCGTKLEIK

SEQ ID NO: 497 - protein, 119 aa, synthetic construct
  REGION 1..119: note = VB5.3 murine VH
QVQLQQSGPELVKPGASVKISCKASGYAFSSSWMNWVKQRPGQCLEWIGRIYPGDGDTKYNGKFKGKATLTADKSSSTAYMHLSSLTSVDSAVYFCARRGTGGWYFDVWGAGTTVTVSS

SEQ ID NO: 498 - protein, 111 aa, synthetic construct
  REGION 1..111: note = VB5.3 murine VL
DIVLTQSPASLAVSLGQRATISCRASESVDSSGNSFMHWYQQKPGQPPQLLIYRASNLESGIPARFSGSGSRTDFTLTINPVEADDVATFYCQQSFDDPFTFGCGTKLEIK

SEQ ID NO: 499 - protein, 119 aa, synthetic construct; identical to SEQ ID NO: 466
  REGION 1..119: note = anti-VB 2 murine VH

SEQ ID NO: 500 - protein, 111 aa, synthetic construct; identical to SEQ ID NO: 467
  REGION 1..111: note = anti-VB 2 murine VL

SEQ ID NO: 501 - protein, 117 aa, synthetic construct
  REGION 1..117: note = anti-VB 7.2 murine VH
QVHLQQSGAELARPGASVKMSCKASGYIFTDYTMHWVKQRPGQGLEWIGHINPSSGYSTYNQKFKDKATLTADKSSSTAYMQLSSLTSEDSAVYYCARSLQLGRDYWGQGTTLTVSS

SEQ ID NO: 502 - protein, 107 aa, synthetic construct
  REGION 1..107: note = anti-VB 7.2 murine VL
DIVMTQSHKFMSTSVGDRVSITCKASQDVYTAVAWYQQKPGQSPKLLIYSASNRYTGVPDRFTGSGSGTDFTFTISSVQAEDLAVYYCQQHYTTPRTFGGGTKLEIK

SEQ ID NO: 503 - protein, 120 aa, synthetic construct
  REGION 1..120: note = anti-VB 4 murine VH
QVQLQQSGAELAKPGTSVKLSCKASGYTFTSYYIYWVKQRPGQGLEWLGYIYPGNGGTYYSEKFKGKATFTADTSSNTAYMLLGSLTPEDSAYYFCARGSGDRYNSLAYWGQGTLVTVSS

SEQ ID NO: 504 - protein, 107 aa, synthetic construct
  REGION 1..107: note = anti-VB 4 murine VL
DIQMTQSPHSLSASLGETVSIECLASEGISNFLAWYQQKPGKSPQLLIYYTSSLQDGVPSRFSGSGSGTQYSLKISNMQPEDEGVYYCQQGYKFPRTFGGGTKLELK

SEQ ID NO: 505 - protein, 107 aa, synthetic construct
  REGION 1..107: note = anti-VB 4 murine VL1
DIQMTQSPSSLSASVGDTVTITCLASEGISNFLAWYQQKPGKAPKLLIYYTSSLQDGVPSRFSGSGSGTDFSLTISNLQPEDFGTYYCQQGYKFPRTFGQGTKVEIK

SEQ ID NO: 506 - protein, 120 aa, synthetic construct
  REGION 1..120: note = anti-VB 13.2 murine VH
EVQLQQSGPELVKPGASVKMSCNASGYTFTDYYIHWLKQRHGKGLEWIGIVNTNNGDTNYNQRFKGKASLTVDKSSSTAYMELNSLTSEDSAVFYCARALYTGSYWFAYWGQGTLVTVSA

SEQ ID NO: 507 - protein, 108 aa, synthetic construct
  REGION 1..108: note = anti-VB 13.2 murine VL
EIVLTQSPAVMAASPGEKVTITCSASSSISSTNLHWYQQKSETSPKPWIYGTSNLASGVPVRFSGSGSGTSYSLTISSIEAEDAATYYCQQWSRYPLTFGSGTKLEII

SEQ ID NO: 508 - protein, 118 aa, synthetic construct
  REGION 1..118: note = anti-VB 14 murine VH
EVQLQQSGAELVKPGASVKLSCTASGFKTEDTYMYWVKQRPEQGLEWIGRIDPANGNTKYDPKFQDKATITADSSSNTAYLQLSSLPSEDTAVYYCARGSYYYAMDYWGQGTSVTVSS

SEQ ID NO: 509 - protein, 111 aa, synthetic construct
  REGION 1..111: note = anti-VB 14 murine VL
DIVLTQSPASLAVSLGQRATISCRASESVDSYGNSFMHWYQQKPGQPPKLLIYRASNLESGIPARFSGSGSRTDFTLTINPVEADDVATYYCQQSNEDPYTFGGGTKLEIK

SEQ ID NO: 510 - protein, 116 aa, synthetic construct
  REGION 1..116: note = anti-VB22 murine VH
EVKLLESGGGLVQPGGSLKLSCAASGFDFSRYWMNWVRQAPGKGLEWIGEINSDSNTINYTPSLKDKFIISRDNAKNTLYLQMNKVRSEDTALYYCARGGLLRDVWGAGTTVTVSS

SEQ ID NO: 511 - protein, 109 aa, synthetic construct
  REGION 1..109: note = anti-VB22 murine VL
QAVVTQESALTTSPGETVTLTCRSSTGAVTTSNYANWVQEKPDHLFTGLIGGTNNRAPGVPARESGSLIGDRAALTITGAQTEDEAIYFCALWYSNHWVFGGGTKLTVL

SEQ ID NO: 512 - protein, 116 aa, synthetic construct
  REGION 1..116: note = anti-VB11 murine VH
QVQLQQSGPEVVRPGVSVKISCKGSGYRFTDSAMHWVKQSHAKSLEWIGVISSYNGNTNYNQKFKGKATMTVDKSSSTAYMELARMTSEDSAIYYCARSRDAMDYWGQGTSVTVSS

SEQ ID NO: 513 - protein, 107 aa, synthetic construct
  REGION 1..107: note = anti-VB11 murine VL
DIKMTQSPSSMYASLGERVTITCKASQDINSYLSWFQQKAGKSPKTLIYRANRLVDGVPSRFSGSGSGQDYSLTISSLEYEDMGIYYCLQYDEFPFTFGGGTRLEIK

SEQ ID NO: 514 - protein, 121 aa, synthetic construct
  REGION 1..121: note = anti-VB1 murine VH
QLQLVQSGPELREPGESVKISCKASGYTFTDYIVHWVKQAPGKGLKWMGWINTYTGTPTYADDFEGRFVFSLEASASTANLQISNLKNEDTATYFCARSWRRGIRGIGFDWGQGVMVTVSS

SEQ ID NO: 515 - protein, 107 aa, synthetic construct
  REGION 1..107: note = anti-VB1 murine VL
DVQMTQSPYNLAASPGESVSINCKASKSINKYLAWYQQKPGKPNKLLIYDGSTLQSGIPSRFSGSGSGTDFTLTIRGLEPEDFGLYYCQQHNEYPPTFGAGTKLELK

SEQ ID NO: 516 - protein, 117 aa, synthetic construct
  REGION 1..117: note = anti-VB12 murine VH
EVQLVESGGDLVKPGGSLKLSCAVSGFTFRSYGMSWVRQTPDKRLEWVALISSGGSYTYYTDSVKGRFTISRDNAKNTLYLQMSSLKSEDTAIYYCSRHGGNFFDYWGQGTTLTVSS

SEQ ID NO: 517 - protein, 108 aa, synthetic construct
  REGION 1..108: note = anti-VB12 murine VL
QIVLTQSPSIMSASPGEKVTMTCSVSSSVSYMEIWYQQKSGTSPKRWIYDTSKLASGVPARFSGSGSGTSYSLTISSMEAEDAATYYCQQWSSNPQYTFGGGTKLEIK

SEQ ID NO: 518 - protein, 117 aa, synthetic construct
  REGION 1..117: note = anti-VB8 murine VH
EVQLVESGGGLVQPGGSRKLSCAASGFTFSNFGMHWVRQAPGKGLEWVAYISSGSSTIYYADTLKGRFTISRDNPKNTLFLQMTSLRSEDTAMYYCARRGEGAMDYWGQGTSVTVSS

SEQ ID NO: 519 - protein, 106 aa, synthetic construct
  REGION 1..106: note = anti-VB8 murine VL
ENVLTQSPAIMSASLGEKVTMSCRASSSVNYIYQYQQKSDASPKLWIYYTSNLAPGVPTRFSGSGSGNSYSLTISSMEGEDAATYYCQQFTSSPFTFGSGTKLEIK

SEQ ID NO: 520 - protein, 121 aa, synthetic construct
  REGION 1..121: note = anti-VB13.1 murine VH
DVKLVESGEGLVKPGGSLKLSCAASGFTFRSYAMSWVRQTPEKRLEWVAHISNGGDYIYYADTVKGRFTISRDNARNTLYLQMSSLKSEDTAMYYCTRPSYYSDPWFFDVWGTGTTVTVSS

SEQ ID NO: 521 - protein, 106 aa, synthetic construct
  REGION 1..106: note = anti-VB13.1 murine VL
ENVLTQSPAIMSASPGEKVTMTCSAGSSVSFMEIWYQQKSSTSPKLWIYDTSKLASGVPGRFSGSGSGNSFSLTISMEAEDVAIYYCLQGSGFPLTFGSGTKLEIK

SEQ ID NO: 522 - protein, 118 aa, synthetic construct
  REGION 1..118: note = anti-VB5.1 murine VH
EVQLQQSGPVLVKPGASVRMSCKASGYTFTDYNIHWVKQSHGRSLEWVGYINPYNGRTGYNQKFKAKATLTVDKSSSTAYMDLRSLTSEDSAVYYCARWDGSSYFDYWGQGTTLTVSS

SEQ ID NO: 523 - protein, 106 aa, synthetic construct
  REGION 1..106: note = anti-VB5.1 murine VL
EIVLTQSPAITAASLGQKVTITCSASSSVSYMHWYQQKSGTSPKPWIYEISKLASGVPARFSGSGSGTSYSLTISSMEAEDAAIYYCQQWNYPLLTFGAGTKLELK

SEQ ID NO: 524 - protein, 118 aa, synthetic construct
  REGION 1..118: note = anti-VB9 murine VH
EVQLQQSVAELVRPGASVKLSCTASGFNIKNTFMHWVKQRPEQGLEWIGRIDPTNGYTKFAPKFQGKATLTAVTSSNTVYLQLSSLTSEDTAIYYCAHDYDAPWFAYWGQGTLVIVSA

SEQ ID NO: 525 - protein, 107 aa, synthetic construct
  REGION 1..107: note = anti-VB9 murine VL
ETTVTQSPASLSVATGEKVTIRCISSTDIDDDMNWYQQKSGEPPKLLISEGNTLRPGVPSRFSSSGYGTDFVFTIENMLSEDVADYYCLQSDNMPLTFGAGTKLELK

SEQ ID NO: 526 - protein, 120 aa, synthetic construct
  REGION 1..120: note = anti-VB17 murine VH
NVQLQESGPGLVKPSQSLSLTCSVAGYSITSGYFWNWIRQFPGNKLEWMGYISYDGSNNYNPSLKNRISITRDTSKNQFFLKLNSVTTEDTATYYCASPSPGTGYAVDYWGQGTSVTVSS

SEQ ID NO: 527 - protein, 112 aa, synthetic construct
  REGION 1..112: note = anti-VB17 murine VL
NVVMTQTPLSLPVSLGDQASISCRSSQSLVHSNGNTYLHWYLQKPGQSPKFLIYKVSNRFSGVPDRFSGGGSGTEFTLKISRVEAEDLGVYFCSQSTHVPFTFGSGTKLEIK

SEQ ID NO: 528 - protein, 121 aa, synthetic construct
  REGION 1..121: note = anti-VB3 murine VH
QIQLQQSGPEVVKPGASVQISCKASGYTFSGSWMNWVKQRPGKGLEWIGRIYPGDGDTDYSGKFKGRATLTADKSSSTAYMRLSSLTSEDSAVYFCARSGYFNYVPVFDYWGQGTTLSVSS

SEQ ID NO: 529 - protein, 106 aa, synthetic construct
  REGION 1..106: note = anti-VB3 murine VL
QIVLTQSPAIMSASLGEEIALTCSANSTVGYIEIWYQQKSGTSPKLLIYTTSNLASGVPSRFSGSGSGTFYSLTISSVEAEDAADYFCHQWSFYPTFGGGTKLEIK

SEQ ID NO: 530 - protein, 123 aa, synthetic construct
  REGION 1..123: note = anti-VB7.1 murine VH
EVQLVESGGGLVKPGGSLKLSCAASGFTFSDYYMYWVRQTPEKRLEWVATISGGGSYTYSPDSVKGRFTISRDNAKNNLYLQMSSLRSEDTAMYFCARERDIYYGNFNAMVYWGRGTSVTVSS

SEQ ID NO: 531 - protein, 111 aa, synthetic construct
  REGION 1..111: note = anti-VB7.1 murine VL
DIVLTQSPVSLTVSLGQRATISCRASKSVSTSGYSYMHWYQQKPGQPPKLLIYLASNLESGVPARFSGSGSGTDFTLNIHPVEEEDAATYYCQHSRDLPWTFGGGTKLEIK

SEQ ID NO: 532 - protein, 119 aa, synthetic construct
  REGION 1..119: note = anti-VB5.3 murine VH
QVQLQQSGPELVKPGASVKISCKASGYAFSSSWMNWVKQRPGQGLEWIGRIYPGDGDTKYNGKFKGKATLTADKSSSTAYMHLSSLTSVDSAVYFCARRGTGGWYFDVWGAGTTVTVSS

SEQ ID NO: 533 - protein, 111 aa, synthetic construct
  REGION 1..111: note = anti-VB5.3 murine VL
DIVLTQSPASLAVSLGQRATISCRASESVDSSGNSFMHWYQQKPGQPPQLLIYRASNLESGIPARFSGSGSRTDFTLTINPVEADDVATFYCQQSFDDPFTFGSGTKLEIK

SEQ ID NO: 534 - protein, 117 aa, synthetic construct
  REGION 1..117: note = VB20 humanized VH 1
EVQLVQSGAEVKKPGASVKLSCTASGFNIKDYYIHWVRQAPGQCLEWMGRIDPEDGETNYAPKFQGRVTISADTSSNTAFLQLSSLTSEDTAVYYCARSDLILFAYWGQGTLVTVSS

SEQ ID NO: 535 - protein, 117 aa, synthetic construct
  REGION 1..117: note = VB20 humanized VH 2
EVQLVQSGAEVKKPGATVKISCKVSGFNIKDYYMHWVQQAPGKCLEWMGLVDPEDGETIYAEKFQGRVTITADTSTDTAYMELSSLRSEDTAVYYCATSDLILFAYWGQGTLVTVSS

SEQ ID NO: 536 - protein, 117 aa, synthetic construct
  REGION 1..117: note = VB20 humanized VH 3
QVQLVQSGAEVKKPGASVKVSCKASGFNIKDYYMHWVRQAPGQCLEWMGRIDPEDGETNYAQKFQGRVTMTRDTSISTAYMELSRLRSDDTAVYYCARSDLILFAYWGQGTLVTVSS

SEQ ID NO: 537 - protein, 121 aa, synthetic construct
  REGION 1..121: note = VB18 humanized VH 1
EVQLLESGGGLVQPGGSLRLSCAASGFDFSRYWMTWVRQAPGKCLEWVSNINSDSSTINYADSVKGRFTISRDNAKNSLYLQMSSLRAEDTALYYCASPYYYGSSSWFAYWGQGTLVTVSS

SEQ ID NO: 538 - protein, 121 aa, synthetic construct
  REGION 1..121: note = VB18 humanized VH 2
EVQLVESGGGLVQPGGSLRLSCAASGFDFSRYSMNWVRQAPGKCLEWVSYINSDSSTIYYADSVKGRFTISRDNAKNSLYLQMNSLRAEDTAVYYCARPYYYGSSSWFAYWGQGTLVTVSS

SEQ ID NO: 539 - protein, 121 aa, synthetic construct
  REGION 1..121: note = VB18 humanized VH 3
EVQLVESGGGLVKPGGSLRLSCAASGFDFSRYSMNWVRQAPGKCLEWVSSINSDSSTIYYADSVKGRFTISRDNAKNSLYLQMNSLRAEDTAVYYCARPYYYGSSSWFAYWGQGTLVTVSS

SEQ ID NO: 540 - protein, 122 aa, synthetic construct
  REGION 1..122: note = VB13.6 humanized VH 1
QVQLVQSGPELKKPGASVKISCKASGYSFTGNYMHWVRQAPGKCLEWMGWINPNTGGTTYAQKLKGRVTLTVDKSSSTAYMELSSLTSEDTAVYYCARSEYYGIYYYAMDYWGQGTTVTVSS

SEQ ID NO: 541 - protein, 122 aa, synthetic construct
  REGION 1..122: note = VB13.6 humanized VH 2
QVQLVQSGAEVKKPGASVKVSCKASGYSFTGNYMHWVRQAPGQCLEWMGWINPNTGGTNYAQKFQGRVTMTRDTSISTAYMELSRLRSDDTAVYYCARSEYYGIYYYAMDYWGQGTTVTVSS

SEQ ID NO: 542 - protein, 116 aa, synthetic construct
  REGION 1..116: note = VB16 humanized VH 1
QVQLVQSGTELKKPGASVKVSCKASGYTFTSYYMHWVRQAPGQCLEWMGWINPSNGGTYYAQKFQGRVTLTRDTSSSTAYMELSSLTSEDTAVYYCARDSYYFDYWGQGTLVTVSS

SEQ ID NO: 543 - protein, 116 aa, synthetic construct
  REGION 1..116: note = VB16 humanized VH 2
QVQLVQSGAEVKKPGASVKVSCKASGYTFTSYYMHWVRQAPGQCLEWMGIINPSNGGTSYAQKFQGRVTMTRDTSTSTVYMELSSLRSEDTAVYYCARDSYYFDYWGQGTLVTVSS

SEQ ID NO: 544 - protein, 116 aa, synthetic construct
  REGION 1..116: note = VB16 humanized VH 3
QVQLVQSGAEVKKPGASVKVSCKASGYTFTSYYMHWVRQAPGQCLEWMGWINPSNGGTNYAQKFQGRVTMTRDTSISTAYMELSRLRSDDTAVYYCARDSYYFDYWGQGTLVTVSS

SEQ ID NO: 545 - protein, 112 aa, synthetic construct
  REGION 1..112: note = VB20 humanized VL 1
DVVMTQTPLSLPVTLGQPASISCRSSQSIVHSNRYTYLEWYLQKPGQSPQLLIYGVSNRFSGVPDRFSGSGSGTDFTLKISRVEAEDVGVYYCFQGTHVPFMFGCGTKLEIK

SEQ ID NO: 546 - protein, 112 aa, synthetic construct
  REGION 1..112: note = VB20 humanized VL 2
DIVMTQTPLSLSVTPGQPASISCRSSQSIVHSNRYTYLEWYLQKPGQSPQLLIYGVSNRFSGVPDRFSGSGSGTDFTLKISRVEAEDVGVYYCFQGTHVPFMFGCGTKLEIK

SEQ ID NO: 547 - protein, 112 aa, synthetic construct
  REGION 1..112: note = VB20 humanized VL 3
DVVMTQSPLSLPVTLGQPASISCRSSQSIVHSNRYTYLEWFQQRPGQSPRRLIYGVSNRFSGVPDRFSGSGSGTDFTLKISRVEAEDVGVYYCFQGTHVPFMFGCGTKLEIK

SEQ ID NO: 548 - protein, 107 aa, synthetic construct
  REGION 1..107: note = VB18 humanized VL 1
DIVLTQSPATLSVSPGDRVTLSCRASQSISNFLHWYQQKPGQSPRLLIKYASQSISGIPSRFSGSGSGTDFTLTINSLEPEDFAVYFCQQSNSWPLTFGCGTKLEIK

SEQ ID NO: 549 - protein, 107 aa, synthetic construct
  REGION 1..107: note = VB18 humanized VL 2
EIVLTQSPATLSLSPGERATLSCRASQSISNFLHWYQQKPGQAPRLLIYYASQSISGIPARFSGSGSGTDFTLTISSLEPEDFAVYYCQQSNSWPLTFGCGTKLEIK

SEQ ID NO: 550 - protein, 107 aa, synthetic construct
  REGION 1..107: note = VB18 humanized VL 3
EIVLTQSPDFQSVTPKEKVTITCRASQSISNFLHWYQQKPDQSPKLLIKYASQSISGVPSRFSGSGSGTDFTLTINSLEAEDAATYYCQQSNSWPLTFGCGTKLEIK

SEQ ID NO: 551 - protein, 107 aa, synthetic construct
  REGION 1..107: note = VB13.6 humanized VL 1
DIQMTQSPSSLSASVGDTVTITCRTSENIYSNLVWYQQKPGKAPQLLIYAATNLAEGVPSRFSGSGSGTDFSLTINSLQPEDFGTYYCQHFYGTPRTFGCGTKVEIK

SEQ ID NO: 552 - protein, 107 aa, synthetic construct
  REGION 1..107: note = VB13.6 humanized VL 2
DIQMTQSPSSLSASVGDRVTITCRTSENIYSNLVWYQQKPGKAPKLLIYAATNLAEGVPSRFSGSGSGTDFTLTISSLQPEDFATYYCQHFYGTPRTFGCGTKLEIK

SEQ ID NO: 553 - protein, 107 aa, synthetic construct
  REGION 1..107: note = VB16 humanized VL-1 1
DIQMTQSPSSLSASVGDRVTLTCRASQDISGYLSWYQQKPGKAPKLLIYSTSTLDSGVPSRFSGSGSGTDFSLTISSLQSEDFATYYCLQYTTSPYTFGCGTKLEIK

SEQ ID NO: 554 - protein, 107 aa, synthetic construct
  REGION 1..107: note = VB16 humanized VL-1 2
DIQMTQSPSSLSASVGDRVTITCRASQDISGYLSWFQQKPGKAPKSLIYSTSTLDSGVPSRFSGSGSGTDFTLTISSLQPEDFATYYCLQYTTSPYTFGCGTKLEIK

SEQ ID NO: 555 - protein, 107 aa, synthetic construct
  REGION 1..107: note = VB16 humanized VL-1 3
DIQMTQSPSSLSASVGDRVTITCRASQDISGYLSWYQQKPGKVPKLLIYSTSTLDSGVPSRFSGSGSGTDFTLTISSLQPEDVATYYCLQYTTSPYTFGCGTKLEIK

SEQ ID NO: 556 - protein, 108 aa, synthetic construct
  REGION 1..108: note = VB16 humanized VL-3 1
DIQMTQSPSTLSASVGDRVTITCKASDHINNWLAWYQQKPGKAPRLLIYGATSLETGVPSRFSGSGSGTDFTLTITSLQPEDVATYYCQQYWSTPIFTFGCGTKLEIK

SEQ ID NO: 557 - protein, 108 aa, synthetic construct
  REGION 1..108: note = VB16 humanized VL-3 2
DIQMTQSPSSVSASVGDRVTITCKASDHINNWLAWYQQKPGKAPKLLIYGATSLETGVPSRFSGSGSGTDFTLTISSLQPEDFATYYCQQYWSTPIFTFGCGTKLEIK

SEQ ID NO: 558 - protein, 108 aa, synthetic construct
  REGION 1..108: note = VB16 humanized VL-3 3
DIQMTQSPSSLSASVGDRVTITCKASDHINNWLAWYQQKPGKAPKSLIYGATSLETGVPSRFSGSGSGTDFTLTISSLQPEDFATYYCQQYWSTPIFTFGCGTKLEIK

SEQ ID NO: 559 - protein, 117 aa, synthetic construct
  REGION 1..117: note = anti-VB20 humanized VH 1
EVQLVQSGAEVKKPGASVKLSCTASGFNIKDYYIHWVRQAPGQGLEWMGRIDPEDGETNYAPKFQGRVTISADTSSNTAFLQLSSLTSEDTAVYYCARSDLILFAYWGQGTLVTVSS

SEQ ID NO: 560 - protein, 117 aa, synthetic construct
  REGION 1..117: note = anti-VB20 humanized VH 2
EVQLVQSGAEVKKPGATVKISCKVSGFNIKDYYMHWVQQAPGKGLEWMGLVDPEDGETIYAEKFQGRVTITADTSTDTAYMELSSLRSEDTAVYYCATSDLILFAYWGQGTLVTVSS

SEQ ID NO: 561 - protein, 117 aa, synthetic construct
  REGION 1..117: note = anti-VB20 humanized VH 3
QVQLVQSGAEVKKPGASVKVSCKASGFNIKDYYMHWVRQAPGQGLEWMGRIDPEDGETNYAQKFQGRVTMTRDTSISTAYMELSRLRSDDTAVYYCARSDLILFAYWGQGTLVTVSS

SEQ ID NO: 562 - protein, 121 aa, synthetic construct
  REGION 1..121: note = anti-VB18 humanized VH 1
EVQLLESGGGLVQPGGSLRLSCAASGFDFSRYWMTWVRQAPGKGLEWVSNINSDSSTINYADSVKGRFTISRDNAKNSLYLQMSSLRAEDTALYYCASPYYYGSSSWFAYWGQGTLVTVSS

SEQ ID NO: 563 - protein, 121 aa, synthetic construct
  REGION 1..121: note = anti-VB18 humanized VH 2
EVQLVESGGGLVQPGGSLRLSCAASGFDFSRYSMNWVRQAPGKGLEWVSYINSDSSTIYYADSVKGRFTISRDNAKNSLYLQMNSLRAEDTAVYYCARPYYYGSSSWFAYWGQGTLVTVSS

SEQ ID NO: 564 - protein, 121 aa, synthetic construct
  REGION 1..121: note = anti-VB18 humanized VH 3
EVQLVESGGGLVKPGGSLRLSCAASGFDFSRYSMNWVRQAPGKGLEWVSSINSDSSTIYYADSVKGRFTISRDNAKNSLYLQMNSLRAEDTAVYYCARPYYYGSSSWFAYWGQGTLVTVSS

SEQ ID NO: 565 - protein, 122 aa, synthetic construct
  REGION 1..122: note = anti-VB13.6 humanized VH 1
QVQLVQSGPELKKPGASVKISCKASGYSFTGNYMHWVRQAPGKGLEWMGWINPNTGGTTYAQKLKGRVTLTVDKSSSTAYMELSSLTSEDTAVYYCARSEYYGIYYYAMDYWGQGTTVTVSS

SEQ ID NO: 566 - protein, 122 aa, synthetic construct
  REGION 1..122: note = anti-VB13.6 humanized VH 2
QVQLVQSGAEVKKPGASVKVSCKASGYSFTGNYMHWVRQAPGQGLEWMGWINPNTGGTNYAQKFQGRVTMTRDTSISTAYMELSRLRSDDTAVYYCARSEYYGIYYYAMDYWGQGTTVTVSS

SEQ ID NO: 567 - protein, 116 aa, synthetic construct
  REGION 1..116: note = anti-VB16 humanized VH 1
QVQLVQSGTELKKPGASVKVSCKASGYTFTSYYMHWVRQAPGQGLEWMGWINPSNGGTYYAQKFQGRVTLTRDTSSSTAYMELSSLTSEDTAVYYCARDSYYFDYWGQGTLVTVSS

SEQ ID NO: 568 - protein, 116 aa, synthetic construct
  REGION 1..116: note = anti-VB16 humanized VH 2
QVQLVQSGAEVKKPGASVKVSCKASGYTFTSYYMHWVRQAPGQGLEWMGIINPSNGGTSYAQKFQGRVTMTRDTSTSTVYMELSSLRSEDTAVYYCARDSYYFDYWGQGTLVTVSS

SEQ ID NO: 569 - protein, 116 aa, synthetic construct
  REGION 1..116: note = anti-VB16 humanized VH 3
QVQLVQSGAEVKKPGASVKVSCKASGYTFTSYYMHWVRQAPGQGLEWMGWINPSNGGTNYAQKFQGRVTMTRDTSISTAYMELSRLRSDDTAVYYCARDSYYFDYWGQGTLVTVSS

SEQ ID NO: 570 - protein, 112 aa, synthetic construct
  REGION 1..112: note = anti-VB20 humanized VL 1
DVVMTQTPLSLPVTLGQPASISCRSSQSIVHSNRYTYLEWYLQKPGQSPQLLIYGVSNRFSGVPDRFSGSGSGTDFTLKISRVEAEDVGVYYCFQGTHVPFMFGQGTKLEIK

SEQ ID NO: 571 - protein, 112 aa, synthetic construct
  REGION 1..112: note = anti-VB20 humanized VL 2
DIVMTQTPLSLSVTPGQPASISCRSSQSIVHSNRYTYLEWYLQKPGQSPQLLIYGVSNRFSGVPDRFSGSGSGTDFTLKISRVEAEDVGVYYCFQGTHVPFMFGQGTKLEIK

SEQ ID NO: 572 - protein, 112 aa, synthetic construct
  REGION 1..112: note = anti-VB20 humanized VL 3
DVVMTQSPLSLPVTLGQPASISCRSSQSIVHSNRYTYLEWFQQRPGQSPRRLIYGVSNRFSGVPDRFSGSGSGTDFTLKISRVEAEDVGVYYCFQGTHVPFMFGQGTKLEIK

SEQ ID NO: 573 - protein, 107 aa, synthetic construct
  REGION 1..107: note = anti-VB18 humanized VL 1
DIVLTQSPATLSVSPGDRVTLSCRASQSISNFLHWYQQKPGQSPRLLIKYASQSISGIPSRFSGSGSGTDFTLTINSLEPEDFAVYFCQQSNSWPLTFGQGTKLEIK

SEQ ID NO: 574 - protein, 107 aa, synthetic construct
  REGION 1..107: note = anti-VB18 humanized VL 2
EIVLTQSPATLSLSPGERATLSCRASQSISNFLHWYQQKPGQAPRLLIYYASQSISGIPARFSGSGSGTDFTLTISSLEPEDFAVYYCQQSNSWPLTFGQGTKLEIK

SEQ ID NO: 575 - protein, 107 aa, synthetic construct
  REGION 1..107: note = anti-VB18 humanized VL 3
EIVLTQSPDFQSVTPKEKVTITCRASQSISNFLHWYQQKPDQSPKLLIKYASQSISGVPSRFSGSGSGTDFTLTINSLEAEDAATYYCQQSNSWPLTFGQGTKLEIK

SEQ ID NO: 576 - protein, 107 aa, synthetic construct
  REGION 1..107: note = anti-VB13.6 humanized VL 1
DIQMTQSPSSLSASVGDTVTITCRTSENIYSNLVWYQQKPGKAPQLLIYAATNLAEGVPSRFSGSGSGTDFSLTINSLQPEDFGTYYCQHFYGTPRTFGQGTKVEIK

SEQ ID NO: 577 - protein, 107 aa, synthetic construct
  REGION 1..107: note = anti-VB13.6 humanized VL 2
DIQMTQSPSSLSASVGDRVTITCRTSENIYSNLVWYQQKPGKAPKLLIYAATNLAEGVPSRFSGSGSGTDFTLTISSLQPEDFATYYCQHFYGTPRTFGQGTKLEIK

SEQ ID NO: 578 - protein, 107 aa, synthetic construct
  REGION 1..107: note = anti-VB16 humanized VL-1 1
DIQMTQSPSSLSASVGDRVTLTCRASQDISGYLSWYQQKPGKAPKLLIYSTSTLDSGVPSRFSGSGSGTDFSLTISSLQSEDFATYYCLQYTTSPYTFGQGTKLEIK

SEQ ID NO: 579 - protein, 107 aa, synthetic construct
  REGION 1..107: note = anti-VB16 humanized VL-1 2
DIQMTQSPSSLSASVGDRVTITCRASQDISGYLSWFQQKPGKAPKSLIYSTSTLDSGVPSRFSGSGSGTDFTLTISSLQPEDFATYYCLQYTTSPYTFGQGTKLEIK

SEQ ID NO: 580 - protein, 107 aa, synthetic construct
  REGION 1..107: note = anti-VB16 humanized VL-1 3
DIQMTQSPSSLSASVGDRVTITCRASQDISGYLSWYQQKPGKVPKLLIYSTSTLDSGVPSRFSGSGSGTDFTLTISSLQPEDVATYYCLQYTTSPYTFGQGTKLEIK

SEQ ID NO: 581 - protein, 108 aa, synthetic construct
  REGION 1..108: note = anti-VB16 humanized VL-3 1
DIQMTQSPSTLSASVGDRVTITCKASDHINNWLAWYQQKPGKAPRLLIYGATSLETGVPSRFSGSGSGTDFTLTITSLQPEDVATYYCQQYWSTPIFTFGQGTKLEIK

SEQ ID NO: 582 - protein, 108 aa, synthetic construct
  REGION 1..108: note = anti-VB16 humanized VL-3 2
DIQMTQSPSSVSASVGDRVTITCKASDHINNWLAWYQQKPGKAPKLLIYGATSLETGVPSRFSGSGSGTDFTLTISSLQPEDFATYYCQQYWSTPIFTFGQGTKLEIK

SEQ ID NO: 583 - protein, 108 aa, synthetic construct
  REGION 1..108: note = anti-VB16 humanized VL-3 3
DIQMTQSPSSLSASVGDRVTITCKASDHINNWLAWYQQKPGKAPKSLIYGATSLETGVPSRFSGSGSGTDFTLTISSLQPEDFATYYCQQYWSTPIFTFGQGTKLEIK

SEQ ID NO: 584 - protein, 7 aa, synthetic construct
  REGION 1..7: note = anti-VB20 VH CDR1
GFNIKDY

SEQ ID NO: 585 - protein, 6 aa, synthetic construct
  REGION 1..6: note = anti-VB20 VH CDR2
DPEDGE

SEQ ID NO: 586 - protein, 8 aa, synthetic construct
  REGION 1..8: note = anti-VB20 VH CDR3
SDLILFAY

SEQ ID NO: 587 - protein, 7 aa, synthetic construct
  REGION 1..7: note = anti-VB18 VH CDR1
GFDFSRY

SEQ ID NO: 588 - protein, 6 aa, synthetic construct
  REGION 1..6: note = anti-VB18 VH CDR2
NSDSST

SEQ ID NO: 589 - protein, 12 aa, synthetic construct
  REGION 1..12: note = anti-VB18 VH CDR3
PYYYGSSSWFAY

SEQ ID NO: 590 - protein, 7 aa, synthetic construct
  REGION 1..7: note = anti-VB13.6 VH CDR1
GYSFTGN

SEQ ID NO: 591 - protein, 6 aa, synthetic construct
  REGION 1..6: note = anti-VB13.6 VH CDR2
NPNTGG

SEQ ID NO: 592 - protein, 13 aa, synthetic construct
  REGION 1..13: note = anti-VB13.6 VH CDR3
SEYYGIYYYAMDY

SEQ ID NO: 593 - protein, 7 aa, synthetic construct
  REGION 1..7: note = anti-VB16 VH CDR1
GYTFTSY

SEQ ID NO: 594 - protein, 6 aa, synthetic construct
  REGION 1..6: note = anti-VB16 VH CDR2
NPSNGG

SEQ ID NO: 595 - protein, 7 aa, synthetic construct
  REGION 1..7: note = anti-VB16 VH CDR3
DSYYFDY

SEQ ID NO: 596 - protein, 16 aa, synthetic construct
  REGION 1..16: note = anti-VB20 VL CDR1
RSSQSIVHSNRYTYLE

SEQ ID NO: 597 - protein, 7 aa, synthetic construct
  REGION 1..7: note = anti-VB20 VL CDR2
GVSNRFS

SEQ ID NO: 598 - protein, 9 aa, synthetic construct
  REGION 1..9: note = anti-VB20 VL CDR3
FQGTHVPFM

SEQ ID NO: 599 - protein, 11 aa, synthetic construct
  REGION 1..11: note = anti-VB18 VL CDR1
RASQSISNFLH

SEQ ID NO: 600 - protein, 7 aa, synthetic construct
  REGION 1..7: note = anti-VB18 VL CDR2
YASQSIS

SEQ ID NO: 601 - protein, 9 aa, synthetic construct
  REGION 1..9: note = anti-VB18 VL CDR3
QQSNSWPLT

SEQ ID NO: 602 - protein, 11 aa, synthetic construct
  REGION 1..11: note = anti-VB13.6 VL CDR1
RTSENIYSNLV

SEQ ID NO: 603 - protein, 7 aa, synthetic construct
  REGION 1..7: note = anti-VB13.6 VL CDR2
AATNLAE

SEQ ID NO: 604 - protein, 9 aa, synthetic construct
  REGION 1..9: note = anti-VB13.6 VL CDR3
QHFYGTPRT

SEQ ID NO: 605 - protein, 11 aa, synthetic construct
  REGION 1..11: note = anti-VB16 VL CDR1
RASQDISGYLS

SEQ ID NO: 606 - protein, 7 aa, synthetic construct
  REGION 1..7: note = anti-VB16 VL CDR2
STSTLDS

SEQ ID NO: 607 - protein, 9 aa, synthetic construct
  REGION 1..9: note = anti-VB16 VL CDR3
LQYTTSPYT

SEQ ID NO: 608 - protein, 11 aa, synthetic construct
  REGION 1..11: note = anti-VB16 VL-3 CDR1
KASDHINNWLA

SEQ ID NO: 609 - protein, 7 aa, synthetic construct
  REGION 1..7: note = anti-VB16 VL-3 CDR2
GATSLET

SEQ ID NO: 610 - protein, 10 aa, synthetic construct
  REGION 1..10: note = anti-VB16 VL-3 CDR3
QQYWSTPIFT

SEQ ID NO: 611 - protein, 117 aa, synthetic construct
  REGION 1..117: note = VB20 murine VH
EVQLQQSGAELVKPGASVKLSCTASGFNIKDYYIHWVNQRSKQCLEWIGRIDPEDGETNYAPKFQGKATISADTSSNTAFLQLSSLTSEDTAVYYCARSDLILFAYWGQGTLVTVSA

SEQ ID NO: 612 - protein, 112 aa, synthetic construct
  REGION 1..112: note = VB20 murine VL
DVVMTQTPLSLPVSLGDQASISCRSSQSIVHSNRYTYLEWYLQKPGQSLKLLIYGVSNRFSGVPDRFSGSGSGTDFTLKISRVEAEDMGVYYCFQGTHVPFMFGCGTKLELK

SEQ ID NO: 613 - protein, 121 aa, synthetic construct
  REGION 1..121: note = VB18 murine VH
EVKLLQSGGGLVQPGGSLKLSCAASGFDFSRYWMTWVRRAPGKCLEWIGEINSDSSTINYSPSLNDKFIISRDNAKNTLYLQMSKVRSEDTALYYCASPYYYGSSSWFAYWGQGTLVTVSA

SEQ ID NO: 614 - protein, 107 aa, synthetic construct
  REGION 1..107: note = VB18 murine VL
DIVLTQSPATLSVTPGDRVSLSCRASQSISNFLHWYQQKSHESPRLLIKYASQSISGIPSRFSGSGSGTDFTLSINSVETEDFGMYFCQQSNSWPLTFGCGTKLELK

SEQ ID NO: 615 - protein, 122 aa, synthetic construct
  REGION 1..122: note = VB13.6 murine VH
EVQLQQSGPELVKPGASVKISCKASGYSFTGNYMNWVKQSPEKCLEWIGEINPNTGGTTYNQKLKAKATLTVDKSSSTAYMQLKSLTSEDSAVYYCARSEYYGIYYYAMDYWGQGTSVTVSS

SEQ ID NO: 616 - protein, 107 aa, synthetic construct
  REGION 1..107: note = VB13.6 murine VL
DIQMTQSPASLSASVGETVTMTCRTSENIYSNLVWYQQKQGKSPQFLVYAATNLAEGVPSRFSGSGSGTQFSLKINSLQPEDFGSYYCQHFYGTPRTFGCGTKLEIK

SEQ ID NO: 617 - protein, 116 aa, synthetic construct
  REGION 1..116: note = VB16 murine VH
QVQLQQPGTELVKPGASVKLSCKASGYTFTSYWMHWVKQRPGQCLEWIGNINPSNGGTYYNEKFKTKATLTVDISSSTAYMQLSSLTSEDSAVYYCARDSYYFDYWGQGTTLAVSS

SEQ ID NO: 618 - protein, 107 aa, synthetic construct
  REGION 1..107: note = VB16 murine VL-1
DIQMTQSPSSLSASLGERVSLTCRASQDISGYLSWLQQKPDGTIKRLIYSTSTLDSGVPKRFSGSRSGSDYSLTISSLESEDFADYYCLQYTTSPYTFGCGTKLEIK

SEQ ID NO: 619 - protein, 108 aa, synthetic construct
  REGION 1..108: note = VB16 murine VL-3
DIQMIQSSSYLSVSLGDRVTITCKASDHINNWLAWYQQKPGNAPRLLISGATSLETGVPSRFSGSGSGKDYTLSITSLQTEDVATYYCQQYWSTPIFTFGCGTKLEIK

SEQ ID NO: 620 - protein, 117 aa, synthetic construct
  REGION 1..117: note = anti-VB20 murine VH
EVQLQQSGAELVKPGASVKLSCTASGFNIKDYYIHWVNQRSKQGLEWIGRIDPEDGETNYAPKFQGKATISADTSSNTAFLQLSSLTSEDTAVYYCARSDLILFAYWGQGTLVTVSA

SEQ ID NO: 621 - protein, 112 aa, synthetic construct
  REGION 1..112: note = anti-VB20 murine VL
DVVMTQTPLSLPVSLGDQASISCRSSQSIVHSNRYTYLEWYLQKPGQSLKLLIYGVSNRFSGVPDRFSGSGSGTDFTLKISRVEAEDMGVYYCFQGTHVPFMFGAGTKLELK

SEQ ID NO: 622 - protein, 121 aa, synthetic construct
  REGION 1..121: note = anti-VB18 murine VH
EVKLLQSGGGLVQPGGSLKLSCAASGFDFSRYWMTWVRRAPGKGLEWIGEINSDSSTINYSPSLNDKFIISRDNAKNTLYLQMSKVRSEDTALYYCASPYYYGSSSWFAYWGQGTLVTVSA

SEQ ID NO: 623 - protein, 107 aa, synthetic construct
  REGION 1..107: note = anti-VB18 murine VL
DIVLTQSPATLSVTPGDRVSLSCRASQSISNFLHWYQQKSHESPRLLIKYASQSISGIPSRFSGSGSGTDFTLSINSVETEDFGMYFCQQSNSWPLTFGAGTKLELK

SEQ ID NO: 624 - protein, 122 aa, synthetic construct
  REGION 1..122: note = anti-VB13.6 murine VH
EVQLQQSGPELVKPGASVKISCKASGYSFTGNYMNWVKQSPEKSLEWIGEINPNTGGTTYNQKLKAKATLTVDKSSSTAYMQLKSLTSEDSAVYYCARSEYYGIYYYAMDYWGQGTSVTVSS

SEQ ID NO: 625 - protein, 107 aa, synthetic construct
  REGION 1..107: note = anti-VB13.6 murine VL
DIQMTQSPASLSASVGETVTMTCRTSENIYSNLVWYQQKQGKSPQFLVYAATNLAEGVPSRFSGSGSGTQFSLKINSLQPEDFGSYYCQHFYGTPRTFGGGTKLEIK

SEQ ID NO: 626 - protein, 116 aa, synthetic construct
  REGION 1..116: note = anti-VB16 murine VH
QVQLQQPGTELVKPGASVKLSCKASGYTFTSYWMHWVKQRPGQGLEWIGNINPSNGGTYYNEKFKTKATLTVDISSSTAYMQLSSLTSEDSAVYYCARDSYYFDYWGQGTTLAVSS

SEQ ID NO: 627 - protein, 107 aa, synthetic construct
  REGION 1..107: note = anti-VB16 murine VL-1
DIQMTQSPSSLSASLGERVSLTCRASQDISGYLSWLQQKPDGTIKRLIYSTSTLDSGVPKRFSGSRSGSDYSLTISSLESEDFADYYCLQYTTSPYTFGGGTKLEIK

SEQ ID NO: 628 - protein, 108 aa, synthetic construct
  REGION 1..108: note = anti-VB16 murine VL-3
DIQMIQSSSYLSVSLGDRVTITCKASDHINNWLAWYQQKPGNAPRLLISGATSLETGVPSRFSGSGSGKDYTLSITSLQTEDVATYYCQQYWSTPIFTFGSGTKLEIK

SEQ ID NO: 629 - protein, 506 aa, synthetic construct
  REGION 1..506: note = full car murine vb1
MLRLLLALNLFPSIQVTGQLQLVQSGPELREPGESVKISCKASGYTFTDYIVHWVKQAPGKCLKWMGWINTYTGTPTYADDFEGRFVFSLEASASTANLQISNLKNEDTATYFCARSWRRGIRGIGFDWGQGVMVTVSSGSTSGSGKPGSGEGSTKGDVQMTQSPYNLAASPGESVSINCKASKSINKYLAWYQQKPGKPNKLLIYDGSTLQSGIPSRFSGSGSGTDFTLTIRGLEPEDFGLYYCQQHNEYPPTFGCGTKLELKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 630 - protein, 502 aa, synthetic construct
  REGION 1..502: note = full car murine vb7.2
MLRLLLALNLFPSIQVTGQVHLQQSGAELARPGASVKMSCKASGYIFTDYTMHWVKQRPGQCLEWIGHINPSSGYSTYNQKFKDKATLTADKSSSTAYMQLSSLTSEDSAVYYCARSLQLGRDYWGQGTTLTVSSGSTSGSGKPGSGEGSTKGDIVMTQSHKFMSTSVGDRVSITCKASQDVYTAVAWYQQKPGQSPKLLIYSASNRYTGVPDRFTGSGSGTDFTFTISSVQAEDLAVYYCQQHYTTPRTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 631 - protein, 507 aa, synthetic construct
  source 1..507: note = 1..507; note = full car murine vb13.6
  REGION 1..507: note = full car murine vb13.6
MLRLLLALNLFPSIQVTGEVQLQQSGPELVKPGASVKISCKASGYSFTGNYMNWVKQSPEKCLEWIGEINPNTGGTTYNQKLKAKATLTVDKSSSTAYMQLKSLTSEDSAVYYCARSEYYGIYYYAMDYWGQGTSVTVSSGSTSGSGKPGSGEGSTKGDIQMTQSPASLSASVGETVTMTCRTSENIYSNLVWYQQKQGKSPQFLVYAATNLAEGVPSRFSGSGSGTQFSLKINSLQPEDFGSYYCQHFYGTPRTFGCGTKLEIKGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS